(19) United States
(12) Patent Application Publication
(10) Pub. No.: US 2006/0004084 A1
(43) Pub. Date: Jan. 5, 2006

(54) (3Z)-3-(2,3-dihydro-1H-inden-1-ylidene)-1,3-dihydro-2H-indol-2-ones as kinase inhibitors

(76) Inventors: Steven W. Andrews, Longmont, CO (US); Xialing Guo, Irvine, CA (US); Zhen Zhu, Tustin, CA (US); Clarence E. Hull III, Trabuco Canyon, CA (US); Julie A. Wurster, Irvine, CA (US); Shimiao Wang, Irvine, CA (US); Edward H. Wang, Dove Canyon, CA (US); Thomas Malone, Irvine, CA (US)

Correspondence Address: ALLERGAN, INC., Legal Department, 2525 Dupont Drive, Irvine, CA, 92612, US

(21) Appl. No.: 11/180,496

(22) Filed: Jul. 12, 2005

Related U.S. Application Data
(63) Continuation-in-part of application No. 10/405,577, filed on Apr. 1, 2003.
Application No. 10/405,577 is a continuation-in-part of application No. 10/116,309, filed on Apr. 3, 2002, now Pat. No. 6,541,504.

(51) Int. Cl. A61K 31/675; A61K 31/404; C07D 45/04

(52) U.S. Cl. 514/414; 548/454; 514/80

(57) ABSTRACT

The present invention relates to organic, molecules capable of modulating tyrosine kinase signal transduction in order to regulate, modulate and/or inhibit abnormal cell proliferation.

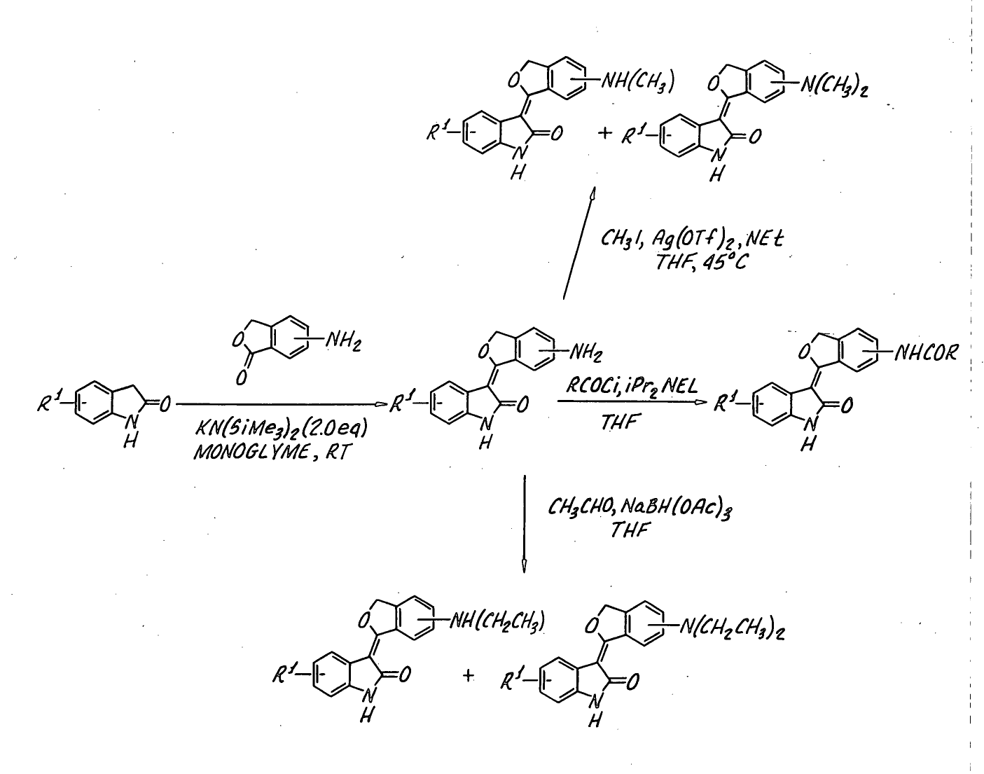

### CROSS REFERENCE TO RELATED APPLICATIONS

This patent application is a continuation in part of Ser. No. 10/405,577 which was filed on Apr. 1, 2003 which is a continuation in part of Ser. No. 10/116,309 which was filed on Apr. 3, 2002, both of which applications are hereby incorporated by reference in their entirety.

### BACKGROUND OF THE INVENTION

1. Field of the Invention

The present invention relates to novel compounds capable of modulating, regulating and/or inhibiting tyrosine kinase signal transduction. The present invention is also directed to methods of regulating, modulating or inhibiting tyrosine kinases, whether of the receptor or non-receptor class, for the prevention and/or treatment of disorders related to unregulated tyrosine kinase signal transduction, including cell growth, metabolic, and blood vessel proliferative disorders.

2. Description of the Related Art

Protein tyrosine kinases (PTKs) comprise a large and diverse class of proteins having enzymatic activity. The PTKs play an important role in the control of cell growth and differentiation.

For example, receptor tyrosine kinase mediated signal transduction is initiated by extracellular interaction with a specific growth factor (ligand), followed by receptor dimerization, transient stimulation of the intrinsic protein tyrosine kinase activity and phosphorylation. Binding sites are thereby created for intracellular signal transduction molecules and lead to the formation of complexes with a spectrum of cytoplasmic signaling molecules that facilitate the appropriate cellular response (e.g., cell division, metabolic homeostasis, and responses to the extracellular microenvironment).

With respect to receptor tyrosine kinases, it has been shown also that tyrosine phosphorylation sites function as high-affinity binding sites for SH2 (src homology) domains of signaling molecules. Several intracellular substrate proteins that associate with receptor tyrosine kinases (RTKs) have been identified. They may be divided into two principal groups: (1) substrates which have a catalytic domain; and (2) substrates which lack such domain but serve as adapters and associate with catalytically active molecules. The specificity of the interactions between receptors or proteins and SH2 domains of their substrates is determined by the amino acid residues immediately surrounding the phosphorylated tyrosine residue. Differences in the binding affinities between SH2 domains and the amino acid sequences surrounding the phosphotyrosine residues on particular receptors are consistent with the observed differences in their substrate phosphorylation profiles. These observations suggest that the function of each receptor tyrosine kinase is determined not only by its pattern of expression and ligand availability but also by the array of downstream signal transduction pathways that are activated by a particular receptor. Thus, phosphorylation provides an important regulatory step which determines the selectivity of signaling pathways recruited by specific growth factor receptors, as well as differentiation factor receptors.

Aberrant expression or mutations in the PTKs have been shown to lead to either uncontrolled cell proliferation (e.g. malignant tumor growth) or to defects in key developmental processes. Consequently, the biomedical community has expended significant resources to discover the specific biological role of members of the PTK family, their function in differentiation processes, their involvement in tumorigenesis and in other diseases, the biochemical mechanisms underlying their signal transduction pathways activated upon ligand stimulation and the development of novel drugs.

Tyrosine kinases can be of the receptor-type (having extracellular, transmembrane and intracellular domains) or the non-receptor type (being wholly intracellular).

The RTKs comprise a large family of transmembrane receptors with diverse biological activities. The intrinsic function of RTKs is activated upon ligand binding, which results in phophorylation of the receptor and multiple cellular substrates, and subsequently in a variety of cellular responses.

At present, at least nineteen (19) distinct RTK subfamilies have been identified. One RTK subfamily, designated the HER subfamily, is believed to be comprised of EGFR, HER2, HER3 and HER4. Ligands to the Her subfamily of receptors include epithelial growth factor (EGF), TGF-α, amphiregulin, HB-EGF, betacellulin and heregulin.

A second family of RTKs, designated the insulin subfamily, is comprised of the INS-R, the IGF-1R and the IR-R. A third family, the “PDGF” subfamily includes the PDGF α and β receptors, CSFIR, c-kit and FLK-II. Another subfamily of RTKs, identified as the FLK family, is believed to be comprised of the Kinase insert Domain-Receptor fetal liver kinase-1 (KDR/FLK-1), the fetal liver kinase 4 (FLK-4) and the fms-like tyrosine kinase 1 (flt-1). Each of these receptors was initially believed to be receptors for hematopoietic growth factors. Two other subfamilies of RTKs have been designated as the FGF receptor family (FGFR1, FGFR2, FGFR3 and FGFR4) and the Met subfamily (c-met and Ron).

Because of the similarities between the PDGF and FLK subfamilies, the two subfamilies are often considered together. The known RTK subfamilies are identified in Plowman et al, 1994, DN&P 7(6): 334-339, which is incorporated herein by reference.

The non-receptor tyrosine kinases represent a collection of cellular enzymes which lack extracellular and transmembrane sequences. At present, over twenty-four individual non-receptor tyrosine kinases, comprising eleven (11) subfamilies (Src, Frk, Btk, Csk, Abl, Zap70, Fes/Fps, Fak, Jak, Ack and LIMK) have been identified. At present, the Src subfamily of non-receptor tyrosine kinases is comprised of the largest number of PTKs and include Src, Yes, Fyn, Lyn, Lck, Blk, Hck, Fgr and Yrk. The Src subfamily of enzymes has been linked to oncogenesis. A more detailed discussion of non-receptor tyrosine kinases is provided in Bolen, 1993, Oncogen 8: 2025-2031, which is incorporated herein by reference.

Many of the tyrosine kinases, whether an RTK or non-receptor tyrosine kinase, have been found to be involved in cellular signaling pathways leading to cellular signal cascades leading to pathogenic conditions, including cancer, psoriasis and hyper immune response.

In view of the surmised importance of PTKs to the control, regulation and modulation of cell proliferation the diseases and disorders associated with abnormal cell proliferation, many attempts have been made to identify receptor and non-receptor tyrosine kinase “inhibitors” using a variety of approaches, including the use of mutant ligands (U.S. Pat. No. 4,966,849), soluble receptors and antibodies is (PCT Application No. WO 94/10202; Kendall & Thomas, 1994, Proc. Nat'l Acad. Sci 90: 10705-09; Kim, et al, 1993, Nature 362: 841-844), RNA ligands (Jellinek, et al, Biochemistry 33: 10450-56); Takano, et al, 1993, Mol. Bio. Cell 4:358A; Kinsella, et al, 1992, Exp. Cell Res. 199: 56-62; Wright, et al, 1992, J. Cellular Phys. 152: 448-57) and tyrosine kinase inhibitors (PCT Application Nos. WO 94/03427; WO 92/21660; WO 91/15495; WO 94/14808; U.S. Pat. No. 5,330,992; Mariani, et al, 1994, Proc. Am. Assoc. Cancer Res. 35: 2268).

More recently, attempts have been made to identify small molecules which act as tyrosine kinase inhibitors. For example, bis monocyclic, bicyclic or heterocyclic aryl compounds (PCT Application No. WO 92/20642), vinylene-azaindole derivatives (PCT Application No. WO 94/14808) and 1-cyclopropyl-4-pyridyl-quinolones (U.S. Pat. No. 5,330,992) have been described generally as tyrosine kinase inhibitors. Styryl compounds (U.S. Pat. No. 5,217,999), styryl-substituted pyridyl compounds (U.S. Pat. No. 5,302,606), certain quinazoline derivatives (EP Application No. 0 566 266 A1), seleoindoles and selenides (PCT Application No. WO 94/03427), tricyclic polyhydroxylic compounds (PCT Application No. WO 92/21660) and benzylphosphonic acid compounds (PCT Application No. WO 91/15495) have been described as compounds for use as tyrosine kinase inhibitors for use in the treatment of cancer.

The identification of effective small compounds which specifically inhibit signal transduction by modulating the activity of receptor and non-receptor tyrosine kinases to regulate and modulate abnormal or inappropriate cell proliferation is therefore desirable and one object of this invention.

Finally, certain small compounds are disclosed in U.S. Pat. Nos. 5,792,783; 5,834,504; 5,883,113; 5,883,116 and 5,886,020 as useful for the treatment of diseases related to unregulated TKS transduction. These patents are hereby incorporated by reference in its entirety for the purpose of disclosing starting materials and methods for the preparation thereof, screens and assays to determine a claimed compound's ability to modulate, regulate and/or inhibit cell proliferation, indications which are treatable with said compounds, formulations and routes of administration, effective dosages, etc.

### BRIEF SUMMARY OF THE INVENTION

The present invention relates to organic molecules capable of modulating, regulating and/or inhibiting tyrosine kinase signal transduction. Such compounds are useful for the treatment of diseases related to unregulated TKS transduction, including cell proliferative diseases such as cancer, atherosclerosis, restenosis, metabolic diseases such as diabetes, inflammatory diseases such as psoriasis and chronic obstructive pulmonary disease, vascular proliferative disorders such as diabetic retinopathy, age-related macular degeneration and retinopathy of prematurity, autoimmune diseases and transplant rejection.

In one illustrative embodiment, the compounds of the present invention have the formula: 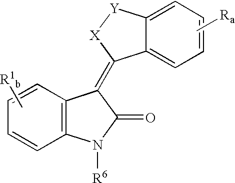
wherein X is O;
Y is [C(R²)₂]_{c};
R¹ is selected from the group consisting of halogen, aryl, C₁ to C₈ alkyl, CF₃, OCF₃, OCF₂H, S(O)_{f}R², (CR³R⁴)_{d}C(O)OR², O(CR³R⁴)ₑC(O)OR², NR²(CR³R⁴)_{d}C(O)R², NR²(CR³R⁴)_{d}C(O)OR², OP(O)(OR²)₂, OC(O)OR², OCH₂O, NR²(CH₂)ₑN(R²)₂, O(CH₂)ₑN(R²)₂, (CR³R⁴)_{d}CN, O(CR³R⁴)ₑCN, (CR³R⁴)_{d}Ar, NR²(CR³R⁴)_{d}Ar, O(CR³R⁴)_{d}Ar, S(O)_{f}(CR³R⁴)_{d}Ar, (CR³R⁴)_{d}SO₂R², (CR³R⁴)_{d}C(O)N(R²)₂, NR²(CR³R⁴)_{d}C(O)N(R²)₂, O(CR³R⁴)_{d}C(O)N(R²)₂, S(O)_{f}(CR³R⁴)ₑC(O)N(R²)₂, (CR³R⁴)_{d}OR², NR²(CR³R⁴)ₑOR², O(CR³R⁴)ₑOR², S(O)_{f}(CR³R⁴)_{d}OR², C(O)(CR³R⁴)_{d}R³, NR²C(O)(CR³R⁴)_{d}R³, OC(O)(CR³R⁴)_{d}N(R²)₂, C(O)(CR³R⁴)_{d}N(R²)₂.NR²C(O)(CR³R⁴)_{d}N(R²)₂, OC(O)(CR³R⁴)_{d}N(R²)₂, (CR³R⁴)_{d}R³, NR²(CR³R⁴)_{d}R³, O(CR³R⁴)_{d}R³, S(O)_{f}(CR³R⁴)_{d}R³, (CR³R⁴)_{d}N(R²)₂, NR²(CR³R⁴)ₑN(R²)₂, O(CR³R⁴)ₑN(R²)₂, S(O)_{f}(CR³R⁴)_{d}N(R²)₂, N(R⁵)₂, OR⁵, C(O)R⁵, S(O)_{f}R⁵;
R² is selected from the group consisting of hydrogen, C₁ to C₈ alkyl, C₁ to C₈ alkenyl, C₁ to C₈ alkynyl, C₁ to C₄ alkylol, lower alkylphenyl, phenyl, (CR³R⁴)_{d}Ar, (CR³R⁴)_{d}C(O)OR², (CR³R⁴)_{d}SO₂R², (CR³R⁴)_{d}OR², (CR³R⁴)_{d}OSO₂R, (CR³R⁴)_{d}P(O)(OR²)₂, (CR³R⁴)_{d}R², (CR³R⁴)ₑN(R²)₂, (CR³R⁴)ₑNR²C(O)N(R²)₂;
N(R²)₂ may form a 3-7 membered heterocyclic ring, for example, pyrrolidine, 3-fluoropyrrolidine, piperidine, 4-fluoropiperidine, N-methylpiperazine, morpholine, 2,6-dimethylmorpholine, thiomorpholine. Said heterocyclic ring may be substituted with one or more of R³;
[C(R²)₂]_{c} may form a 3-7 membered carbocyclic or heterocyclic ring;
R is selected from the group consisting of halogen, C₁ to C₈ alkyl, CF₃, OCF₃, OCF₂H, (CR³R⁴)_{d}CN, NR²(CR³R⁴)ₑCN, O(CR³R⁴)ₑCN, S(O)_{f}R², (CR³R⁴)_{d}C(O)OR², NR²(CR³R⁴)_{d}C(O)OR², O(CR³R⁴)_{d}C(O)OR², S(O)_{f}(CR³R⁴)_{d}C(O)OR², (CR³R⁴)_{d}Ar, NR²(CR³R⁴)_{d}Ar, O(CR³R⁴)_{d}Ar, S(O)_{f}(CR³R⁴)_{d}Ar, (CR³R⁴)_{d}SO₂R², NR²(CR³R⁴)_{d}S(O)_{f}R², O(CR³R⁴)_{d}S(O)_{f}R², S(O)_{f}(CR³R⁴)ₑS(O)_{f}R², (CR³R⁴)_{d}C(O)N(R²)₂, NR²(CR³R⁴)_{d}C(O)N(R²)₂, O(CR³R⁴)_{d}C(O)N(R²)₂, S(O)_{f}(CR³R⁴)ₑC(O)N(R²)₂, (CR³R⁴)_{d}OR², NR²(CR³R⁴)ₑOR², O(CR³R⁴)ₑOR², S(O)_{f}(CR³R⁴)_{d}OR², (CR³R⁴)_{d}OSO₂R², NR²(CR³R⁴)ₑOSO₂R², O(CR³R⁴)ₑOSO₂R², S(O)_{f}(CR³R⁴)ₑOSO₂R²(CR³R⁴)_{d}P(O)(OR²)₂, NR²(CR³R⁴)_{d}P(O)(OR²)₂, O(CR³R⁴)_{d}P(O)(OR²)₂, S(O)_{f}(CR³R⁴)ₑP(O)(OR²)₂, C(O)(CR³R⁴)_{d}R³, NR²C(O)(CR³R⁴)_{d}R³, OC(O)(CR³R⁴)_{d}N(R²)₂, C(O)(CR³R⁴)_{d}N(R²)₂, NR²C(O)(CR³R⁴)_{d}N(R²)₂, OC(O)(CR³R⁴)_{d}N(R²)₂, (CR³R⁴)_{d}R³, NR²(CR³R⁴)_{d}R³, O(CR³R⁴)_{d}R³, S(O)_{f}(CR³R⁴)_{d}R³, HNC(O)R², HN—C(O)OR², (CR³R⁴)_{d}N(R²)₂, NR²(CR³R⁴)ₑN(R²)₂, O(CR³R⁴)ₑN(R²)₂, S(O)_{f}(CR³R⁴)_{d}N(R²)₂, OP(O)(OR²)₂, OC(O)OR², OCH₂O, HN—CH═CH, —N(COR²)CH₂CH₂, HC═N—NH, N═CH—S, (CR³R⁴)_{d}C═C(CR³R⁴)_{d}R², (CR³R⁴)_{d}C═C(CR³R⁴)_{d}OR², (CR³R⁴)_{d}C═C(CR³R⁴)_{d}N(R²)₂, (CR³R⁴)_{d}CC(CR³R⁴)_{d}R², (CR³R⁴)_{d}CC(CR³R⁴)ₑOR², (CR³R⁴)_{d}CC(CR³R⁴)ₑN(R²)₂, (CR³R⁴)_{d}C(O)(CR³R⁴)_{d}R², (CR³R⁴)_{d}C(O)(CR³R⁴)_{d}OR², (CR³R⁴)_{d}C(O)(CR³R⁴)_{d}N(R²)₂,
R³ and R⁴ may be selected from the group consisting of H, F, hydroxy, and C₁-C₄ alkyl or CR³R⁴ may represent a carbocyclic or heterocyclic ring of from 3 to 6 carbons, alternatively (CR³R⁴)_{d} and (CR³R⁴)ₑ may form a 3-7 membered carbocyclic or heterocyclic ring, preferably R³ and R⁴ are H, F, CH₃ or hydroxy;
R⁵ is Ar—R¹_{b}
R⁶ is selected from hydrogen, C₁-C₈ alkyl, hydroxylmethyl and phenyl;
b is 0 or an integer of from 1 to 2;
a is 0 or an integer of from 1 to 3;
c is an integer of from 1 to 2;
d is 0 or an integer of from 1 to 5;
e is an integer of from 1 to 4;
f is 0 or an integer of from 1 to 2, and further provided said alkyl or aryl radicals may be substituted with one or two halo, hydroxy, lower alkyloxy, lower alkyl amino or cycloalkylamino radicals wherein the cycloalkyl ring can include an enchained oxygen, sulfur or additional nitrogen atom and may be substituted with one or two halo or lower alkyl radicals;
   and pharmaceutically acceptable salts thereof.

### BRIEF DESCRIPTION OF THE DRAWING FIGURES

The FIGURE shows a schematic of the preparation of the compounds of Examples 1 through 27.

### DETAILED DESCRIPTION OF THE INVENTION

In one embodiment of the present invention R¹ is selected from the group consisting of H, i.e. b is 0; CH₃, F and Cl; preferably R¹ is H, F or Cl.

Preferably, a is 0 or R is selected from the group consisting of NHCOR⁷ and N(R⁷)₂ wherein R⁷ is selected from the group consisting of hydrogen, C₁ to C₄ alkyl and phenyl, wherein said alkyl or phenyl may be substituted with hydroxy, methylol or amino substituents and more preferably R⁷ is selected from the group consisting of hydrogen, methyl, ethyl, hydroxypropyl, and aminomethylol phenyl.

Preferably R⁶ is H.

Preferably c is 1.

In another preferred embodiment of the present invention R¹ is selected from the group consisting of H, i.e. b is 0, F and Cl.

Preferably, a is 1 and R is selected from the group consisting of (CR³R⁴)_{d}N(R²)₂, NR²(CR³R⁴)_{d}N(R²)₂, O(CR³R⁴)_{d}N(R²)₂, (CR³R⁴)_{d}CC(CR³R⁴)_{d}N(R²)₂, NR²C(O)(CR³R⁴)_{d}N(R²)₂.

Preferably R⁶ is H.

Preferably c is 1.

In particular, the compounds of the present invention are selected from the compounds of Tables 1, 3, 4, 5 and 6 and the Examples below.

TABLE 1

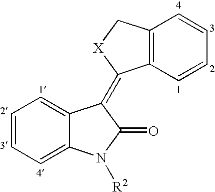

| Example | | | | | | | | | | |
|---|---|---|---|---|---|---|---|---|---|---|
| Number | R² | 1 | 2 | 3 | 4 | 1′ | 2′ | 3′ | 4′ | X |
| Example 1 | H | H | H | H | H | H | H | H | H | O |
| Example 2 | H | H | H | H | H | H | Cl | H | H | O |
| Example 3 | H | H | H | H | H | CH₃ | H | H | H | O |
| Example 4 | H | H | H | H | H | H | F | H | H | O |
| Example 5 | H | H | NH₂ | H | H | H | H | H | H | O |
| Example 6 | H | H | NHCOCH₃ | H | H | H | H | H | H | O |
| Example 7 | H | H | NHCOCH₂CH₂CH₃ | H | H | H | H | H | H | O |
| Example 8 | H | H | NHCO-cyclopropyl | H | H | H | H | H | H | O |
| Example 9 | H | H | NHCOCH₂CH₂CH₂Cl | H | H | H | H | H | H | O |
| Example 10 | H | H | NHCOCH₂Ph-4-OCH₃ | H | H | H | H | H | H | O |
| Example 11 | H | H | NHCH₂CH₃ | H | H | H | H | H | H | O |
| Example 12 | H | H | H | NH₂ | H | H | H | H | H | O |
| Example 13 | H | H | NHCOPh-3-NH₂,6- | H | H | H | H | H | H | O |
| | | | CH₂OH | | | | | | | |
| Example 14 | H | H | NHCH₂CH₂CH₂OH | H | H | H | H | H | H | O |
| Example 15 | H | H | H | NHCH₂CH₃ | H | H | H | H | H | O |
| Example 16 | H | H | NH₂ | H | H | H | Cl | H | H | O |
| Example 17 | H | H | H | NH₂ | H | H | Cl | H | H | O |
| Example 18 | H | H | H | NHCOCH₃ | H | H | H | H | H | O |
| Example 19 | H | H | H | NHCOCH₃ | H | H | Cl | H | H | O |
| Example 20 | H | H | NHCOCH₃ | H | H | H | Cl | H | H | O |
| Example 21 | H | H | N(CH₃)₂ | H | H | H | H | H | H | O |
| Example 22 | H | H | NHCH₃ | H | H | H | H | H | H | O |
| Example 23 | H | H | H | N(CH₃)₂ | H | H | H | H | H | O |
| Example 24 | H | H | H | NHCH₃ | H | H | H | H | H | O |
| Example 26 | H | H | NHCOCH₂CH₂CH₂Cl | H | H | H | H | H | H | O |
| Example 27 | H | H | N(CH₂CH₃)₂ | H | H | H | H | H | H | O |

The present invention is further directed to pharmaceutical compositions comprising a pharmaceutically effective amount of the above-described compounds and a pharmaceutically acceptable carrier or excipient. Such a composition is believed to modulate signal transduction by a tyrosine kinase, either by inhibition of catalytic activity, affinity to ATP or ability to interact with a substrate.

More particularly, the compositions of the present invention may be included in methods for treating diseases comprising proliferation, fibrotic or metabolic disorders, for example cancer, fibrosis, psoriasis, atherosclerosis, arthritis, and other disorders related to abnormal vasculogenesis and/or angiogenesis, such as diabetic retinopathy.

The following defined terms are used throughout this specification:

“Ac” refers to acetyl.

“Ar” refers to aryl.

“Tf” refers to triflate.

“Me” refers to methyl.

“Et” refers to ethyl.

“tBu” refers to t-butyl.

“iPr” refers to I-propyl.

“Ph” refers to phenyl.

“Pharmaceutically acceptable salt” refers to those salts which retain the biological effectiveness and properties of the free bases and which are obtained by reaction with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Certain “pharmaceutically acceptable salts” are the salts of free acid, e.g. the sodium salt of a carboxylic acid.

“Alkyl” refers to a straight-chain, branched or cyclic saturated aliphatic hydrocarbon. Preferably, the alkyl group has 1 to 12 carbons. More preferably, it is a lower alkyl of from 1 to 8 carbons, most preferably 1 to 4 carbons. Typical alkyl groups include methyl, ethyl, propyl, isopropyl, butyl, isobutyl, tertiary butyl, pentyl, hexyl and the like. The alkyl group may be optionally substituted with one or more substituents are selected from the group consisting of hydroxyl, cyano, alkoxy, ═O, ═S, NO₂, halogen, dimethyl amino, and SH.

“Alkenyl” refers to a straight-chain, branched or cyclic unsaturated hydrocarbon group containing at least one carbon-carbon double bond. Preferably, the alkenyl group has 1 to 12 carbons. More preferably it is a lower alkenyl of from 1 to 8 carbons, most preferably 1 to 4 carbons. The alkenyl group may be optionally substituted with one or more substituents selected from the group consisting of hydroxyl, cyano, alkoxy, ═O, ═S, NO₂, halogen, dimethyl amino, and SH.

“Alkynyl” refers to a straight-chain, branched or cyclic unsaturated hydrocarbon containing at least one carbon-carbon triple bond. Preferably, the alkynyl group has 1 to 12 carbons. More preferably it is a lower alkynyl of from 1 to 8 carbons, most preferably 1 to 4 carbons. The alkynyl group may be optionally substituted with one or more substituents selected from the group consisting of hydroxyl, cyano, alkoxy, ═O, ═S, NO₂, halogen, dimethyl amino, and SH.

“Alkoxyl” refers to an “O-alkyl” group.

“Aryl” refers to an aromatic group which has at least one ring having a conjugated pi electron system and includes carbocyclic aryl, heterocyclic aryl and biaryl groups. The aryl group may be optionally substituted with one or more substituents selected from the group consisting of halogen, trihalomethyl, hydroxyl, SH, OH, NO₂, amine, thioether, cyano, alkoxy, alkyl, and amino.

“Alkaryl” refers to an alkyl that is covalently joined to an aryl group. Preferably, the alkyl is a lower alkyl.

“Carbocyclic aryl” refers to an aryl group wherein the ring atoms are carbon.

“Heterocyclic aryl” refers to an aryl group having from 1 to 4 heteroatoms as ring atoms, the remainder of the ring atoms being carbon. Heteroatoms include oxygen, sulfur, and nitrogen. Thus, heterocyclic aryl groups include furanyl, thienyl, pyridyl, pyrrolyl, N-lower alkyl pyrrolo, pyrimidyl, pyrazinyl, imidazolyl, tetrazoyl and the like.

“Hydrocarbyl” refers to a hydrocarbon radical having only carbon and hydrogen atoms. Preferably, the hydrocarbyl radical has from 1 to 20 carbon atoms, more preferably from 1 to 12 carbon atoms and most preferably from 1 to 8 carbon atoms.

“Substituted hydrocarbyl” refers to a hydrocarbyl radical wherein one or more, but not all, of the hydrogen and/or the carbon atoms are replaced by a halogen, nitrogen, oxygen, sulfur or phosphorus atom or a radical including a halogen, nitrogen, oxygen, sulfur or phosphorus atom, e.g. fluoro, chloro, cyano, nitro, hydroxyl, phosphate, thiol, etc.

“Amide” refers to —C(O)—NH—R′ or —NH—C(O)R′, wherein R′ is alkyl, aryl, alkylaryl or hydrogen.

“Thioamide” refers to —C(S)—NH—R′ or —NH—C(S)R′, wherein R′ is alkyl, aryl, alkylaryl or hydrogen.

“Amine” refers to a —N(R″)R′″ group, wherein R″ and R′″ are independently selected from the group consisting of alkyl, aryl, and alkylaryl.

“Thioether” refers to —S—R″, wherein R″ is alkyl, aryl, or alkylaryl.

“Sulfonyl” refers to —S(O)₂—R″″, where R″″ is aryl, C(CN)═C-aryl, CH₂CN, alkyaryl, sulfonamide, NH-alkyl, NH-alkylaryl, or NH-aryl.

The present invention relates to compounds capable of regulating and/or modulating tyrosine kinase signal transduction and more particularly receptor and non-receptor tyrosine kinase signal transduction.

Receptor tyrosine kinase mediated signal transduction is initiated by extracellular interaction with a specific growth factor (ligand), followed by receptor dimerization, transient stimulation of the intrinsic protein tyrosine kinase activity and phosphorylation. Binding sites are thereby created for intracellular signal transduction molecules and lead to the formation of complexes with a spectrum of cytoplasmic signaling molecules that facilitate the appropriate cellular response (e.g., cell division, metabolic effects and responses to the extracellular microenvironment).

It has been shown that tyrosine phosphorylation sites in growth factor receptors function as high-affinity binding sites for SH2 (src homology) domains of signaling molecules. Several intracellular substrate proteins that associate with receptor tyrosine kinases have been identified. They may be divided into two principal groups: (1) substrates which have a catalytic domain; and (2) substrates which lack such domain but serve as adapters and associate with catalytically active molecules. The specificity of the interactions between receptors and SH2 domains of their substrates is determined by the amino acid residues immediately surrounding the phosphorylated tyrosine residue. Differences in the binding affinities between SH2 domains and the amino acid sequences surrounding the phosphotyrosine residues on particular receptors are consistent with the observed differences in their substrate phosphorylation profiles. These observations suggest that the function of each receptor tyrosine kinase is determined not only by its pattern of expression and ligand availability but also by the array of downstream signal transduction pathways that are activated by a particular receptor. Thus, phosphorylation provides an important regulatory step which determines the selectivity of signaling pathways recruited by specific growth factor receptors, as well as differentiation factor receptors.

Tyrosine kinase signal transduction results in, among other responses, cell proliferation, differentiation and metabolism. Abnormal cell proliferation may result in a wide array of disorders and diseases, including the development of neoplasia such as carcinoma, sarcoma, leukemia, glioblastoma, hemangioma, psoriasis, arteriosclerosis, arthritis and diabetic retinopathy (or other disorders related to uncontrolled angiogenesis and/or vasculogenesis, e.g. macular degeneration).

This invention is therefore directed to compounds which regulate, modulate and/or inhibit tyrosine kinase signal transduction by affecting the enzymatic activity of the RTKs and/or the non-receptor tyrosine kinases and interfering with the signal transduced by such proteins. More particularly, the present invention is directed to compounds which regulate, modulate and/or inhibit the RTK and/or non-receptor tyrosine kinase mediated signal transduction pathways as a therapeutic approach to cure many kinds of solid tumors, including but not limited to carcinoma, sarcoma, leukemia, erythroblastoma, glioblastoma, meningioma, astrocytoma, melanoma and myoblastoma. Indications may include, but are not limited to brain cancers, bladder cancers, ovarian cancers, gastric cancers, pancreas cancers, colon cancers, blood cancers, lung cancers and bone cancers.

Biological data for the compounds of the present invention was generated by use of the following assays.

VEGF Stimulated Ca⁺⁺ Signal In Vitro

Automated FLIPR (Fluorometric Imaging Plate Reader) technology was used to screen for inhibitors of VEGF induced increases in intracellular calcium levels in fluorescent dye loaded endothelial cells. HUVEC (human umbilical vein endothelial cells) (Clonetics) were seeded in 96-well fibronectin coated black-walled plates overnight @ 37° C./5% CO₂. Cells were loaded with calcium indicator Fluo-4 for 45 minutes at 37° C. Cells were washed 4 times (Original Cell Wash, Labsystems) to remove extracellular dye. For screening, cells were pre-incubated with test agents for 30 minutes at a single concentration (10 uM) or at concentrations ranging from 0.01 to 10.0 uM, followed by VEGF stimulation (5 ng/mL). Changes in fluorescence at 516 nm were measured simultaneously in all 96 wells using a cooled CCD camera. Data were generated by determining max-min fluorescence levels for unstimulated, stimulated, and drug treated samples. IC₅₀ values for test compounds were calculated from % inhibition of VEGF stimulated responses in the absence of inhibitor.

Protocol for KDR Assay:

The cytoplasmic domain of the human VEGF receptor (VEGFR-2) was expressed as a Histidine-tagged fusion protein following infection of insect cells using an engineered baculovirus. His-VEGFR-2 was purified to homogeneity, as determined by SDS-PAGE, using nickel resin chromatography. Kinase assays were performed in 96 well microtiter plates that were coated overnight with 30 μg of poly-Glu-Tyr (4:1) in 10 mM Phosphate Buffered Saline (PBS), pH 7.2-7.4. The plates were incubated with 1% BSA and then washed four times with PBS prior to starting the reaction. Reactions were carried out in 120 μL reaction volumes containing 3.6 μM ATP in kinase buffer (50 mM Hepes pH 7.4, 20 mM MgCl₂, 0.1 mM MnCl₂ and 0.2 mM Na₃VO₄). Test compounds were reconstituted in 100% DMSO and added to the reaction to give a final DMSO concentration of 5%. Reactions were initiated by the addition 0.5 ng of purified protein. Following a ten minute incubation at 25° C., the reactions were washed four times with PBS containing 0.05% Tween-20. 100 μl of a monoclonal anti-phosphotyrosine antibody-peroxidase conjugate was diluted 1:10000 in PBS-Tween-20 and added to the wells for 30 minutes. Following four washes with PBS-Tween-20, 100 μl of O-Phenylenediamine Dihydrochloride in Phosphate-citrate buffer, containing urea hydrogen peroxide, was added to the wells for 7 minutes as a colorimetric substrate for the peroxidase. The reaction was terminated by the addition of 100 μl of 2.5N H₂SO₄ to each well and read using a microplate ELISA reader set at 492 nm. IC₅₀ values for compound inhibition were calculated directly from graphs of optical density (arbitrary units) versus compound concentration following subtraction of blank values.

The results of said assays are set forth in Table 2, below.

TABLE 2
In vitro VEGF Inhibition

| | VEGF mean | VEGF (% | VEGF mean |
|---|---|---|---|
| | IC₅₀(μM) | inhibition @ 10 uM) | IC₅₀(μM) |
| Example | (Cell based | (Cell based | (Kinase assay, |
| Number | assay, Ca⁺⁺) | assay, Ca⁺⁺) | KDr with BSA) |
| Example 1 | 0.11 | 99 | |
| Example 2 | 0.05 | 98 | |
| Example 3 | 0.685 | 95 | |
| Example 4 | 0.055 | 99 | 0.073 |
| Example 5 | 0.04 | 98 | 0.13 |
| Example 6 | 1.225 | 97 | 1.11 |
| Example 7 | | 5 | 9.78 |
| Example 8 | | 48 | 0.85 |
| Example 9 | | 35 | 3.25 |
| Example 10 | | 50 | 8.02 |
| Example 11 | 0.78 | 99 | 0.66 |
| Example 12 | 0.04 | 98 | 0.065 |
| Example 13 | 2.095 | 99 | 1.34 |
| Example 14 | 0.85 | 100 | 0.64 |
| Example 15 | 0.051 | 95 | 0.037 |
| Example 16 | 0.06 | 99 | 0.015 |
| Example 17 | 0.055 | 98 | 0.139 |
| Example 18 | 0.04 | 97 | 0.063 |
| Example 19 | 0.05 | 99 | 0.066 |
| Example 20 | 0.097 | 99 | 0.39 |
| Example 21 | 1.31 | 94 | 1.69 |
| Example 22 | 0.29 | 96 | 0.17 |
| Example 23 | 0.096 | 96 | 0.043 |
| Example 24 | 0.073 | 98 | 0.061 |
| Example 25 | | | |
| Example 26 | | | |
| Example 27 | | | |

As shown in Table 2, above, the compounds of Examples 1-6, 11-24 and 16-20 are preferred as they show % inhibition of VEGF>90% or VEGF IC₅₀<1.0 μM in either the cell or kinase assay.

### EXAMPLES

### Example 1

### 3-(3H-isobenzofuran-1-ylidene)-1,3-dihydro-indol-2-one

To a suspension of sodium hydride (6.0 g, 150 mmol, 60% in mineral oil) in 300 mL DMF was added oxindole (10.0 g, 75.1 mmol) in 50 mL DMF over 8 min. After stirring for 15 min at room temperature, a solution of phthalide (13.1 g, 97.6 mmol) in 50 mL DMF was added over 1 min. The mixture was stirred for 1.25 h, then poured into 1100 mL H₂O. Addition of 4% aqueous HCl solution gave a yellow solid which was filtered and rinsed with H₂O to give the title compound (8.75 g, 47%).

¹H NMR (500 MHz, DMSO-D6) δ 10.41 (s, 1H), 9.65 (d, J=8.1 Hz, 1H), 7.83 (d, J=7.6 Hz, 1H), 7.65 (m, 2H), 7.55 (m, 1H), 7.10 (ddd, J=7.6, 7.6, 1.0 Hz, 1H), 6.95 (ddd, J=7.6, 7.6, 1.0 Hz, 1H), 6.81 (d, J=7.6 Hz, 1H), 5.81 (s, 2H).

### Example 2

### 5-Chloro-3-(3H-isobenzofuran-1-ylidene)-1,3-dihydro-indol-2-one

To a solution containing 5-chlorooxindole (0.30 g, 1.79 mmol) and phthalide (0.312 g, 2.33 mmol) in 6.0 mL of dimethylformamide (DMF) was added 3.76 mL of sodium hexamethyldisilazane (1.0 M in tetrahydrofuran (THF)) over 1 min. The solution was stirred at room temperature for 25 min and then 85 mg (0.634 mmol) phthalide was added. After an additional 20 min at room temperature the mixture was poured into 70 mL of 4% aqueous HCl solution to give a yellow solid. The aqueous mixture was extracted with EtOAc and the organic phase washed with saturated NaHCO₃, brine and then dried with Na₂SO₄. After removal of the solvent in vacuo, the solid residue was recrystallized from MeOH/EtOAc to afford the title (141 mg, 28%) compound as a yellow solid.

¹H NMR (500 MHz, DMSO-D6) δ ppm 5.87 (s, 2H) 6.84 (d, J=8.30 Hz, 1H) 7.16 (dd, J=8.18, 2.32 Hz, 1H) 7.60 (m, 1H) 7.70 (m, 2H) 7.82 (d, J=2.20 Hz, 1H) 9.65 (d, J=8.06 Hz, 1H) 10.58 (s, 1H).

### Example 3

### 3-(3H-Isobenzofuran-1-ylidene)-4-methyl-1,3-dihydro-indol-2-one

To a solution containing 4-methyloxindole (0.15 g, 1.02 mmol) and phthalide (0.178 g, 1.33 mmol) in 3.0 mL DMF was added 2.14 mL of sodium hexamethyldisilazane (1.0 M in tetrahydrofuran (THF)) over 1 min. The solution was stirred at room temperature for 30 min and then poured into 50 mL of 4% HCl to give a yellow solid. The aqueous mixture was extracted with EtOAc and the organic phase washed with saturated NaHCO₃, H₂O, dilute HCl, brine and the solution dried with Na₂SO₄. The solvent was removed in vacuo and the solid obtained was purified by chromatography (silica gel, CHCl₃/EtOAc, 7:3). The solid obtained was recrystallized from EtOAc/hexanes to afford the title compounds (3.8 mg) as a yellow solid.

¹H NMR (500 MHz, CDCl₃) δ ppm 2.58 (s, 3H) 5.62 (s, 2H) 6.70 (d, J=7.81 Hz, 1H) 6.86 (d, J=7.32 Hz, 1H) 7.07 (t, J=7.57 Hz, 1H) 7.45 (m, 1H) 7.56 (m, 2H) 7.68 (s, 1H) 9.70 (d, J=6.83 Hz, 1H).

### Example 4

### 5-Fluoro-3-(3H-isobenzofuran-1-ylidene)-1,3-dihydro-indol-2-one

To a solution at 0° C. containing 5-fluorooxindole (0.30 g, 1.99 mmol) and phthalide (0.400 g, 2.98 mmol) in 5.0 mL DMF was added 4.2 mL of sodium hexamethyldisilazane (1.0 M in THF) over 5 min. The solution was stirred at room temperature for 3 h and then quenched into cold 1.0 M aqueous HCl solution to give a yellow solid. The solid was collected and then purified by chromatography (silica gel, hexanes/EtOAc, 4:1) to afford the title compound (32 mg, 6%) as a yellow solid.

¹H NMR (500 MHz, DMSO-d₆) δ ppm 5.83 (s, 2H) 6.78 (dd, J=8.42, 4.39 Hz, 1H) 6.93 (m, 1H) 7.57 (m, 2H) 7.68 (m, 2H) 9.64 (d, J=8.05 Hz, 1H) 10.44 (s, 1H)

### Example 5

### 3-(6-Amino-3H-isobenzofuran-1-ylidene)-1,3-dihydro-indol-2-one

To a solution containing oxindole (0.5 g, 3.76 mmol) in 7.5 mL DMF was added 7.51 mL of sodium hexamethyldisilazane (1.0 M in THF) over 3 min. After stirring 10 min at room temperature, a solution of 6-aminophthalide (0.672 g, 4.51 mmol) in 4.0 mL DMF was added over 3 min. The reaction was stirred for 50 min at room temperature and then poured into 4% HCl to give a yellow solid. The solid was filtered to a wet cake and then partitioned between EtOAc and saturated NaHCO₃. Then the mixture was heated to dissolve the solid. The organic phase was washed with H₂O, brine and then dried with Na₂SO₄. The solvent was removed in vacuo and the resultant solid triturated with CHCl₃ to afford the title compound (445 mg, 45%) as a yellow solid.

¹H NMR (500 MHz, DMSO-D6) δ ppm 5.39 (s, 2H) 5.61 (s, 2H) 6.80 (d, J=7.32 Hz, 1H) 6.89 (dd, J=8.06, 2.20 Hz, 1H) 6.93 (td, J=7.57, 0.98 Hz, 1H) 7.08 (td, J=7.69, 1.22 Hz, 1H) 7.28 (d, J=8.30 Hz, 1H) 7.82 (d, J=7.32 Hz, 1H) 8.86 (d, J=1.95 Hz, 1H) 10.32 (s, 1H).

### Example 6

### N-[3-(2-Oxo-1,2-dihydro-indol-3-ylidene)-1,3-dihydro-isobenzofuran-5-yl]-acetamide

To a solution of 3-(6-Amino-3H-isobenzofuran-1-ylidene)-1,3-dihydro-indol-2-one (50.0 mg, 0.189 mmol) and N,N-diisopropylethylamine (98.8 μL, 0.567 mmol) in 2.0 mL THF was added acetyl chloride (13.4 μL, 0.189 mmol). After stirring a troom temperature for 1 h, the slurry was partitioned between NaHCO₃ solution and EtOAc (warmed to dissolve solid). The organic phase was washed with H₂O, 4% aqueout HCl solution, H₂O, saturated NaHCO₃, brine and dried with Na₂SO₄. After concentrating in vacuo the residue was triturated with EtOAc to give the title compound (47.4 mg, 82%) as a yellow solid.

¹H NMR (500 MHz, DMSO-D6) δ ppm 2.09 (s, 3H) 5.75 (s, 2H) 6.83 (d, J=7.81 Hz, 1H) 6.96 (td, J=7.57, 0.98 Hz, 1H) 7.11 (td, J=7.69, 1.22 Hz, 1H) 7.57 (d, J=8.30 Hz, 1H) 7.83 (d, J=7.81 Hz, 1H) 8.10 (dd, J=8.30, 1.95 Hz, 1H) 9.59 (d, J=1.95 Hz, 1H) 10.29 (s, 1H) 10.41 (s, 1H).

### Example 7

### N-[3-(2-Oxo-1,2-dihydro-indol-3-ylidene)-1,3-dihydro-isobenzofuran-5-yl]-butyramide

To a solution of 3-(6-Amino-3H-isobenzofuran-1-ylidene)-1,3-dihydro-indol-2-one (50.0 mg, 0.189 mmol) and N,N-diisopropylethylamine (98.8 μL, 0.567 mmol) in 2.0 mL THF was added butyryl chloride (19.6 μL, 0.189 mmol). After stirring at room temperature for 1 h, the slurry was filtered and rinsed with MeOH and EtOAc/hexanes (1:1) to afford the title compound (46.2 mg, 73%) as a yellow solid.

¹H NMR (500 MHz, DMSO-D6) δ ppm 0.93 (t, J=7.57 Hz, 3H) 1.63 (m, 2H) 2.34 (t, J=7.32 Hz, 2H) 5.75 (s, 2H) 6.83 (d, J=7.81 Hz, 1H) 6.96 (m, 1H) 7.11 (td, J=7.57, 1.46 Hz, 1H) 7.57 (d, J=8.30 Hz, 1H) 7.83 (d, J=7.32 Hz, 1H) 8.11 (dd, J=8.30, 1.46 Hz, 1H) 9.61 (d, J=1.95 Hz, 1H) 10.23 (s, 1H) 10.40 (s, 1H).

### Example 8

### Cyclopropanecarboxylic acid[3-(2-oxo-1,2-dihydro-indol-3-ylidene)-1,3-dihydro-isobenzofuran-5-yl]-amide

To a solution of 3-(6-Amino-3H-isobenzofuran-1-ylidene)-1,3-dihydro-indol-2-one (50.0 mg, 0.189 mmol) and N,N-diisopropylethylamine (98.8 μL, 0.567 mmol) in 2.0 mL THF was added cyclopropane carbonyl chloride (17.2 μL, 0.189 mmol). After stirring at room temperature for 1 h, the slurry was warmed briefly, stirred 10 min at room temperature, filtered and rinsed with MeOH and EtOAc/hexanes (1:1) to afford the title compound (44.7 mg, 71%) as a yellow solid.

¹H NMR (500 MHz, DMSO-D6) δ ppm 0.82 (m, 4H) 1.89 (m, 1H) 5.75 (s, 2H) 6.83 (d, J=7.32 Hz, 1H) 6.96 (m, 1H) 7.12 (m, 1H) 7.57 (d, J=8.30 Hz, 1H) 7.83 (d, J=7.32 Hz, 1H) 8.08 (d, J=8.30 Hz, 1H) 9.63 (d, J=1.46 Hz, 1H) 10.40 (s, 1H) 10.54 (s, 1H).

### Example 9

### 4-Chloro-N-[3-(2-oxo-1,2-dihydro-indol-3-ylidene)-1,3-dihydro-isobenzofuran-5-yl]-butyramide

To a solution of 3-(6-Amino-3H-isobenzofuran-1-ylidene)-1,3-dihydro-indol-2-one (50.0 mg, 0.189 mmol) and N,N-diisopropylethylamine (98.8 μL, 0.567 mmol) in 2.0 mL THF was added 4-chlorobutyryl chloride (21.2 μL, 0.189 mmol). After stirring at room temperature for 1 h, the slurry was filtered and rinsed with MeOH and EtOAc/hexanes (1:1) to afford the title compound (58.8 mg, 84%) as a yellow solid.

¹H NMR (500 MHz, DMSO-D6) δ ppm 2.06 (m, 2H) 2.54 (t, J=7.32 Hz, 2H) 3.72 (t, J=6.35 Hz, 2H) 5.75 (s, 2H) 6.83 (d, J=7.32 Hz, 1H) 6.96 (m, 1H) 7.12 (td, J=7.69, 1.22 Hz, 1H) 7.58 (d, J=8.30 Hz, 1H) 7.83 (d, J=7.32 Hz, 1H) 8.10 (dd, J=8.30, 1.46 Hz, 1H) 9.63 (d, J=1.95 Hz, 1H) 10.34 (s, 1H) 10.40 (s, 1H).

### Example 10

### 2-(4-Methoxy-phenyl)-N-[3-(2-oxo-1,2-dihydro-indol-3-ylidene)-1,3-dihydro-isobenzofuran-5-yl]-acetamide

To a solution of 3-(6-Amino-3H-isobenzofuran-1-ylidene)-1,3-dihydro-indol-2-one (50.0 mg, 0.189 mmol) and N,N-diisopropylethylamine (98.8 μL, 0.567 mmol) in 2.0 mL THF was added 4-methoxyphenylacetyl chloride (28.9 μL, 0.189 mmol). After stirring at room temperature for 1 h, the slurry was warmed briefly, stirred 10 min at room temperature, filtered and rinsed with MeOH and EtOAc/hexanes (1:1) to afford the title compound (48.3 mg, 62%) as a yellow solid.

¹H NMR (500 MHz, DMSO-D6) δ ppm 3.62 (s, 2H) 3.73 (s, 3H) 5.75 (s, 2H) 6.83 (d, J=7.81 Hz, 1H) 6.90 (m, 2H) 6.96 (td, J=7.57, 0.98 Hz, 1H) 7.12 (td, J=7.57, 0.98 Hz, 1H) 7.28 (m, 2H) 7.57 (d, J=8.79 Hz, 1H) 7.83 (d, J=7.32 Hz, 1H) 8.11 (dd, J=8.30, 1.95 Hz, 1H) 9.63 (d, J=1.46 Hz, 1H) 10.40 (s, 1H) 10.47 (s, 1H).

### Example 11

### 3-(6-Ethylamino-3H-isobenzofuran-1-ylidene)-1,3-dihydro-indol-2-one

A mixture of 3-(6-Amino-3H-isobenzofuran-1-ylidene)-1,3-dihydro-indol-2-one (50.0 mg, 0.189 mmol), acetaldehyde (10.0 mg, 0.227 mmol), and sodium triacetoxyborohydride (52.1 mg, 0.246 mmol) was stirred at room temperature for 2.5 h. The reaction was then partitioned between EtOAc and H₂O. The organic phase was washed with H₂O, brine and then dried with Na₂SO₄. The solvent was removed in vacuo and the residue recrystallized from EtOAc/hexanes to afford the title compound (21.6 mg, 39%) as a yellow solid.

¹H NMR (500 MHz, DMSO-D6) δ ppm 1.22 (t, J=7.08 Hz, 3H) 3.12 (m, 2H) 5.63 (s, 2H) 5.91 (t, J=5.37 Hz, 1H) 6.82 (d, J=7.32 Hz, 1H) 6.92 (m, 2H) 7.09 (m, 1H) 7.32 (d, J=8.30 Hz, 1H) 7.82 (d, J=7.32 Hz, 1H) 8.94 (d, J=1.95 Hz, 1H) 10.28 (s, 1H).

### Example 12

### 3-(5-Amino-3H-isobenzofuran-1-ylidene)-1,3-dihydro-indol-2-one

To a solution containing oxindole (0.5 g, 3.76 mmol) in 7.5 mL DMF was added 7.51 mL of sodium hexamethyldisilazane (1.0 M in THF) over 3 min. After stirring 10 min at room temperature, a solution of 5-aminophthalide (0.672 g, 4.51 mmol) in 4.0 mL DMF was added over 3 min. The reaction was stirred for 30 min at room temperature and then poured into 4% aqueous HCl solution to give a yellow cloudy solution. After stirring the mixture 3 min, the solution was made basic by adding saturated NaHCO₃. The yellow solid was filtered, washed with H₂O, and then dissolved in CHCl₃/MeOH. The solvent was removed in vacuo and the solid purified by chromatography (silica gel, CHCl₃/MeOH, 95:5) to give the title compound (345 mg, 35%) as a yellow solid.

¹H NMR (500 MHz, DMSO-D6) δ ppm 5.60 (s, 2H) 6.27 (s, 2H) 6.67 (m, 2H) 6.78 (d, J=7.32 Hz, 1H) 6.89 (m, 1H) 7.01 (td, J=7.57, 1.46 Hz, 1H) 7.74 (d, J=7.32 Hz, 1H) 9.33 (d, J=9.28 Hz, 1H) 10.19 (s, 1H).

### Example 13

### 5-Amino-2-hydroxymethyl-N-[3-(2-oxo-1,2-dihydro-indol-3-ylidene)-1,3-dihydro-isobenzofuran-5-yl]-benzamide

To a solution containing oxindole (1.5 g, 11.3 mmol) in 23 mL DMF was added 22.54 mL of sodium hexamethyldisilazane (1.0 M in THF) over 5 min. After stirring 5 min at rt, a solution of 6-aminophthalide (2.017 g, 13.5 mmol) in 11.0 mL DMF was added over 4 min. The reaction was stirred for 30 min at room temperature and then quenched into 4% aqueous HCl solution. The aqueous solution was neutralized to pH 6 with 1 M NaOH and then made basic with saturated NaHCO₃. The solid was filtered and washed with H₂O and then partitioned between EtOAc and saturated NaHCO₃ (heated to dissolve the solid). The organic phase was washed with H₂O, brine and then dried with Na₂SO₄. The solvent was removed in vacuo and the solid triturated with CHCl₃. The yellow solid was filtered (1.2 g) (Example 5) and the filtrate concentrated in vacuo. The solid (0.88 g) obtained from the filtrate was purified by chromatography (CHCl₃/MeOH, 96:4) to afford the lower R_{f} product, which after trituration with CHCl₃, gave the title compound (7.6 mg) as a yellow solid.

¹H NMR (500 MHz, DMSO-D6) δ ppm 4.48 (d, J=5.37 Hz, 2H) 5.13 (t, J=5.37 Hz, 1H) 5.27 (s, 2H) 5.78 (s, 2H) 6.66 (dd, J=8.30, 2.44 Hz, 1H) 6.83 (m, 2H) 6.96 (t, J=7.57 Hz, 1H) 7.12 (t, J=7.57 Hz, 1H) 7.16 (d, J=8.30 Hz, 1H) 7.62 (d, J=8.30 Hz, 1H) 7.84 (d, J=7.81 Hz, 1H) 8.04 (d, J=8.30 Hz, 1H) 9.83 (s, 1H) 10.35 (s, 1H) 10.70 (s, 1H).

### Example 14

### 3-[6-(3-Hydroxy-propylamino)-3H-isobenzofuran-1-ylidene]-1,3-dihydro-indol-2-one

A mixture of 3-(6-Amino-3H-isobenzofuran-1-ylidene)-1,3-dihydro-indol-2-one (25.0 mg, 0.095 mmol), 3-bromo-1-propanol (85.5 μL, 0.946 mmol), and silver sulfate (59.0 mg, 0.189 mmol) in 0.8 mL DMF was heated at 120° C. for 1.5 h. The mixture was partitioned between EtOAc and H₂O and the organic layer separated from the silver salts. The solution was washed with H₂O, brine and then dried with Na₂SO₄. Concentrating the solution in vacuo gave a residue which was purified by chromatography (silica gel, CHCl₃/MeOH, 96:4) to give the title compound (3 mg, 10%) as a yellow solid.

¹H NMR (500 MHz, CDCl₃) δ ppm 1.92 (m, 2H) 3.23 (t, J=6.59 Hz, 1H) 3.71 (q, J=5.86 Hz, 2H) 4.09 (t, J=6.35 Hz, 2H) 5.72 (s, 2H) 6.88 (d, J=7.32 Hz, 1H) 7.07 (t, J=7.32 Hz, 1H) 7.20 (m, 1H) 7.38 (dd, J=7.81, 1.95 Hz, 1H) 7.47 (s, 1H) 7.51 (d, J=7.81 Hz, 1H) 7.98 (d, J=7.81 Hz, 1H) 8.57 (s, 1H) 9.82 (d, J=2.44 Hz, 1H).

### Example 15

### 3-(5-Ethylamino-3H-isobenzofuran-1-ylidene)-1,3-dihydro-indol-2-one

A mixture of 3-(5-Amino-3H-isobenzofuran-1-ylidene)-1,3-dihydro-indol-2-one (50.0 mg, 0.189 mmol), acetaldehyde (10.0 mg, 0.227 mmol), and sodium triacetoxyborohydride (52.1 mg, 0.246 mmol) was stirred at room temperature for 50.5 h. The reaction was then partitioned between EtOAc and H₂O. The organic phase was washed with H₂O, brine and then dried with Na₂SO₄. The solvent was removed in vacuo and the solid chromatographed (CHCl₃/MeOH, 97.5:2.5) to afford the title compound (14.1 mg, 25%) as a yellow solid.

¹H NMR (500 MHz, CDCl₃) δ ppm 1.31 (t, J=7.08 Hz, 3H) 3.26 (m, 2H) 4.25 (t, J=4.64 Hz, 1H) 5.56 (s, 2H) 6.53 (s, 1H) 6.68 (dd, J=8.79, 1.95 Hz, 1H) 6.85 (d, J=7.81 Hz, 1H) 7.02 (td, J=7.57, 0.98 Hz, 1H) 7.09 (td, J=7.57, 0.98 Hz, 1H) 7.47 (s, 1H) 7.93 (d, J=7.81 Hz, 1H) 9.51 (d, J=8.79 Hz, 1H).

### Example 16

### 3-(6-Amino-3H-isobenzofuran-1-ylidene)-5-chloro-1,3-dihydro-indol-2-one

To a solution of 5-chlorooxindole (0.629 g, 3.78 mmol) in 10.0 mL monoglyme was added 7.51 mL sodium hexamethyldisilazane (1.0 M in THF) over 3 min. After stirring at room temperature for 8 min, a slurry of 6-aminophthalide (0.561 g, 3.78 mmol) in 4.0 mL of monoglyme was added in one portion. The mixture was stirred for 40 min and then quenched into 100 mL of 4% aqueous HCl solution. The yellow solid was filtered and then partitioned between EtOAc and saturated NaHCO₃ (heated to dissolve the solid). The organic phase was washed with H₂O, brine and then dried with Na₂SO₄. The solvent was removed in vacuo and the residue was triturated with MeOH to give the title compound (439 mg, 39%) as a yellow solid.

¹H NMR (500 MHz, DMSO-D6) δ ppm 5.44 (s, 2H) 5.66 (s, 2H) 6.81 (d, J=8.30 Hz, 1H) 6.92 (dd, J=8.30, 1.95 Hz, 1H) 7.12 (dd, J=8.05, 2.20 Hz, 1H) 7.31 (d, J=8.30 Hz, 1H) 7.79 (d, J=1.95 Hz, 1H) 8.83 (d, J=1.95 Hz, 1H) 10.48 (s, 1H).

### Example 17

### 3-(5-Amino-3H-isobenzofuran-1-ylidene)-5-chloro-1,3-dihydro-indol-2-one

To a solution of 5-chlorooxindole (0.629 g, 3.78 mmol) in 10.0 mL monoglyme was added 7.51 mL sodium hexamethyldisilazane (1.0 M in THF) over 3 min. After stirring at room temperature for 8 min, a solution of 5-aminophthalide (0.561 g, 3.78 mmol) in 3.0 mL of DMF was added over 1 min. The mixture was stirred for 40 min and then quenched into 4% aqueous HCl solution. The aqueous solution was neutralized to pH 7 with 1 M NaOH and then made basic with saturated NaHCO₃. The solid was filtered and washed with H₂O and then partitioned between EtOAc and saturated NaHCO₃ (heated to dissolve the solid). The organic phase was washed with H₂O, brine and then dried with Na₂SO₄. The solvent was removed in vacuo and the residue triturated with MeOH to give the title compound (353 mg, 31%) as a yellow solid.

¹H NMR (500 MHz, DMSO-D6) δ ppm 5.64 (s, 2H) 6.39 (s, 2H) 6.68 (m, 2H) 6.77 (d, J=8.30 Hz, 1H) 7.03 (dd, J=8.05, 2.20 Hz, 1H) 7.70 (d, J=1.95 Hz, 1H) 9.31 (m, 1H) 10.34 (s, 1H).

### Example 18

### N-[1-(2-Oxo-1,2-dihydro-indol-3-ylidene)-1,3-dihydro-isobenzofuran-5-yl]-acetamide

To a solution of 3-(5-Amino-3H-isobenzofuran-1-ylidene)-1,3-dihydro-indol-2-one (50.0 mg, 0.189 mmol) and N,N-diisopropylethylamine (98.8 μL, 0.567 mmol) in 2.0 mL THF was added acetyl chloride (13.4 μL, 0.189 mmol). After stirring at room temperature for 3 h, the mixture was concentrated in vacuo and the solid triturated with MeOH. Filtering the mixture and rinsing with MeOH and hexanes/EtOAc (7:3) afforded the title compound (42.2 mg, 73%) as a yellow solid.

¹H NMR (500 MHz, DMSO-D6) δ ppm 2.12 (s, 3H) 5.77 (s, 2H) 6.82 (d, J=7.32 Hz, 1H) 6.94 (td, J=7.57, 0.98 Hz, 1H) 7.08 (td, J=7.57, 0.98 Hz, 1H) 7.54 (dd, J=8.79, 1.95 Hz, 1H) 7.80 (d, J=7.32 Hz, 1H) 8.09 (s, 1H) 9.55 (d, J=8.79 Hz, 1H) 10.37 (s, 1H) 10.43 (s, 1H).

### Example 19

### N-[1-(5-Chloro-2-oxo-1,2-dihydro-indol-3-ylidene)-1,3-dihydro-isobenzofuran-5-yl]-acetamide

To a solution of 3-(5-Amino-3H-isobenzofuran-1-ylidene)-5-chloro-1,3-dihydro-indol-2-one (56.5 mg, 0.189 mmol) and N,N-diisopropylethylamine (98.8 μL, 0.567 mmol) in 2.5 mL THF was added acetyl chloride (13.4 μL, 0.189 mmol). After stirring at room temperature for 3 h, the mixture was concentrated in vacuo and the solid triturated with MeOH. Filtering the mixture and rinsing with MeOH and hexanes/EtOAc (7:3) afforded the title compound (55.9 mg, 87%) as a yellow solid.

¹H NMR (500 MHz, DMSO-D6) δ ppm 2.12 (s, 3H) 5.82 (s, 2H) 6.82 (d, J=7.81 Hz, 1H) 7.12 (dd, J=8.30, 2.44 Hz, 1H) 7.56 (dd, J=8.54, 1.71 Hz, 1H) 7.76 (d, J=2.44 Hz, 1H) 8.10 (s, 1H) 9.52 (d, J=8.79 Hz, 1H) 10.46 (s, 1H) 10.52 (s, 1H).

### Example 20

### N-[3-(5-Chloro-2-oxo-1,2-dihydro-indol-3-ylidene)-1,3-dihydro-isobenzofuran-5-yl]-acetamide

To a solution of 3-(6-Amino-3H-isobenzofuran-1-ylidene)-5-chloro-1,3-dihydro-indol-2-one (56.5 mg, 0.189 mmol) and N,N-diisopropylethylamine (98.8 μL, 0.567 mmol) in 2.5 mL THF was added acetyl chloride (13.4 μL, 0.189 mmol). After stirring at room temperature for 3 h, the mixture was filtered and rinsed with isopropanol and hexanes/EtOAc (7:3) to give the title compound (46.9 mg, 73%) as a yellow solid.

¹H NMR (500 MHz, DMSO-D6) δ ppm 2.09 (s, 3H) 5.79 (s, 2H) 6.83 (d, J=8.30 Hz, 1H) 7.15 (dd, J=8.30, 1.95 Hz, 1H) 7.60 (d, J=8.30 Hz, 1H) 7.80 (d, J=1.95 Hz, 1H) 8.11 (dd, J=8.30, 1.95 Hz, 1H) 9.58 (d, J=1.95 Hz, 1H) 10.29 (s, 1H) 10.55 (s, 1H).

### Example 21 and Example 22

### 3-(6-Dimethylamino-3H-isobenzofuran-1-ylidene)-1,3-dihydro-indol-2-one and 3-(6-Methylamino-3H-isobenzofuran-1-ylidene)-1,3-dihydro-indol-2-one

To a solution of 3-(6-Amino-3H-isobenzofuran-1-ylidene)-1,3-dihydro-indol-2-one (50.0 mg, 0.189 mmol) and N,N-diisopropylethylamine (65.8 μL, 0.378 mmol) in 2.0 mL THF was added iodomethane (12.9 μL, 0.208 mmol). After stirring at room temperature for 21 h, silver triflate (53.4 mg, 0.208 mmol) was added and the mixture heated at 45° C. for 16 h. The mixture was partitioned between EtOAc and saturated NaHCO₃ and the organic separated. The organic layer was washed with H₂O, brine and then dried with Na₂SO₄. The solution was evaporated in vacuo and the residue purified by chromatography (silica gel, 2% MeOH/CHCl₃) to give 3-(6-Dimethylamino-3H-isobenzofuran-1-ylidene)-1,3-dihydro-indol-2-one (3.6 mg) as a yellow solid.

¹H NMR (500 MHz, CDCl₃) δ ppm 3.08 (s, 6H) 5.62 (s, 2H) 6.86 (d, J=7.81 Hz, 1H) 6.97 (dd, J=8.30, 2.44 Hz, 1H) 7.05 (td, J=7.57, 0.98 Hz, 1H) 7.14 (td, J=7.57, 1.46 Hz, 1H) 7.30 (d, J=8.30 Hz, 1H) 7.62 (s, 1H) 8.00 (d, J=6.83 Hz, 1H) 9.31 (d, J=2.44 Hz, 1H) and 3-(6-Methylamino-3H-isobenzofuran-1-ylidene)-1,3-dihydro-indol-2-one (6.3 mg) as a yellow solid.

¹H NMR (500 MHz, CDCl₃) δ ppm 2.96 (s, 3H) 4.02 (s, 1H) 5.60 (s, 2H) 6.84 (m, 2H) 7.05 (td, J=7.57, 0.98 Hz, 1H) 7.14 (td, J=7.57, 1.46 Hz, 1H) 7.24 (obsc d, 1H) 7.52 (s, 1H) 7.99 (d, J=7.81 Hz, 1H) 9.10 (d, J=2.44 Hz, 1H).

### Example 23 and Example 24

### 3-(5-Dimethylamino-3H-isobenzofuran-1-ylidene)-1,3-dihydro-indol-2-one and 3-(5-Methylamino-3H-isobenzofuran-1-ylidene)-1,3-dihydro-indol-2-one

To a solution of 3-(5-Amino-3H-isobenzofuran-1-ylidene)-1,3-dihydro-indol-2-one (50.0 mg, 0.189 mmol) and N,N-diisopropylethylamine (65.8 μL, 0.378 mmol) in 2.0 mL THF was added iodomethane (12.9 μL, 0.208 mmol). After stirring at room temperature for 21 h, silver triflate (53.4 mg, 0.208 mmol) was added and the mixture heated at 45° C. for 16 h. The mixture was partitioned between EtOAc and saturated NaHCO₃ and the organic separated. The organic layer was washed with H₂O, brine and then dried with Na₂SO₄. The solution was evaporated in vacuo and chromatographed with 2% MeOH/CHCl₃ to give 3-(5-Dimethylamino-3H-isobenzofuran-1-ylidene)-1,3-dihydro-indol-2-one (1.2 mg) as a yellow-orange solid and 3-(5-Methylamino-3H-isobenzofuran-1-ylidene)-1,3-dihydro-indol-2-one (8.9 mg) as an orange solid.

Example 23: ¹H NMR (500 MHz, CDCl₃) δ ppm 3.10 (s, 6H) 5.59 (s, 2H) 6.62 (d, J=2.44 Hz, 1H) 6.81 (dd, J=8.79, 2.44 Hz, 1H) 6.85 (d, J=7.32 Hz, 1H) 7.02 (td, J=7.57, 1.46 Hz, 1H) 7.09 (td, J=7.57, 0.98 Hz, 1H) 7.47 (s, 1H) 7.93 (d, J=7.32 Hz, 1H) 9.54 (d, J=9.28 Hz, 1H);

Example 24: ¹H NMR (500 MHz, DMSO-d₆) δ ppm 2.79 (s, 3H) 5.63 (s, 2H) 6.65 (s, 1H) 6.69 (dd, J=9.03, 2.20 Hz, 1H) 6.78 (d, J=7.81 Hz, 1H) 6.85 (br, 1H) 6.89 (t, J=7.08 Hz, 1H) 7.01 (m, 1H) 7.75 (d, J=7.81 Hz, 1H) 9.38 (d, J=8.79 Hz, 1H) 10.20 (s, 1H) LR MS (EI): 278 (M⁺)

### Example 26

### 4-Chloro-N-[3-(2-oxo-1,2-dihydro-indol-3-ylidene)-1,3-dihydro-isobenzofuran-5-yl]-butyramide

To a solution of 3-(6-Amino-3H-isobenzofuran-1-ylidene)-1,3-dihydro-indol-2-one (50.0 mg, 0.189 mmol) and N,N-diisopropylethylamine (98.8 μL, 0.567 mmol) in 2.0 mL THF was added 4-chlorobutryl chloride (21.2 μL, 0.189 mmol). After stirring at room temperature for 1 h, the slurry was filtered and rinsed with MeOH and EtOAc/hexanes (1:1) to afford the title compound (58.8 mg, 84%) as a yellow solid.

¹H NMR (500 MHz, DMSO-D6) δ ppm 2.06 (m, 2H) 2.54 (t, J=7.32 Hz, 2H) 3.72 (t, J=6.35 Hz, 2H) 5.75 (s, 2H) 6.83 (d, J=7.32 Hz, 1H) 6.96 (m, 1H) 7.12 (td, J=7.69, 1.22 Hz, 1H) 7.58 (d, J=8.30 Hz, 1H) 7.83 (d, J=7.32 Hz, 1H) 8.10 (dd, J=8.30, 1.46 Hz, 1H) 9.63 (d, J=1.95 Hz, 1H) 10.34 (s, 1H) 10.40 (s, 1H)

### Example 11 and Example 27

### 3-(6-Ethylamino-3H-isobenzofuran-1-ylidene)-1,3-dihydro-indol-2-one and 3-(6-Diethylamino-3H-isobenzofuran-1-ylidene)-1,3-dihydro-indol-2-one

A mixture of 3-(6-Amino-3H-isobenzofuran-1-ylidene)-1,3-dihydro-indol-2-one (581 mg, 2.20 mmol), acetaldehyde (116 mg, 2.64 mmol), and sodium triacetoxyborohydride (606 mg, 2.86 mmol) was stirred at room temperature for 3 h. The reaction was then partitioned between ethyl acetate and H₂O. The organic phase was washed with dilute aqueous NaHCO₃ solution, H₂O, brine and then dried with Na₂SO₄. The solvent was removed in vacuo and the residue was dissolved in CHCl₃/MeOH and purified by chromatography (silica gel, hexanes/EtOAc, 7:3) to give 3-(6-Ethylamino-3H-isobenzofuran-1-ylidene)-1,3-dihydro-indol-2-one (395.1 mg, 61%) as a yellow solid and 3-(6-Diethylamino-3H-isobenzofuran-1-ylidene)-1,3-dihydro-indol-2-one (32.9 mg, 5%) as a yellow solid.

Example 27: ¹H NMR (500 MHz, DMSO-D6) δ ppm 1.15 (t, J=7.08 Hz, 6H) 3.42 (q, J=7.16 Hz, 4H) 5.66 (s, 2H) 6.84 (d, J=7.81 Hz, 1H) 6.95 (t, J=7.57 Hz, 1H) 7.00 (dd, J=8.54, 2.20 Hz, 1H) 7.10 (t, J=7.57 Hz, 1H) 7.40 (d, J=8.30 Hz, 1H) 7.83 (d, J=7.81 Hz, 1H) 9.22 (d, J=2.44 Hz, 1H) 10.28 (s, 1H).

The compounds of Tables 3, 4 and 5 are prepared by procedures analogous to the procedures used to prepare the compounds of Examples 1 through 27 and as specifically set forth in Examples 28 through 134. These compounds, like the compounds of Examples 1 through 27, show activity as VEGF inhibitors.

TABLE 3

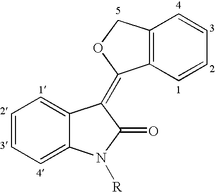

| Example | | | | | | | | | | |
|---|---|---|---|---|---|---|---|---|---|---|
| Number | 1 | 2 | 3 | 4 | 5 | 1′ | 2′ | 3′ | 4′ | R |
| 28 | H | H | H | H | H,H | H | OMe | H | H | H |
| 29 | H | 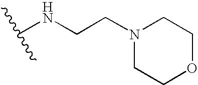 | H | H | H,H | H | H | H | H | H |
| 30 | H | NHCOCH₂Br | H | H | H,H | H | Cl | H | H | H |
| 31 | H | 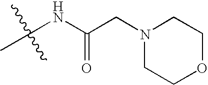 | H | H | H,H | H | Cl | H | H | H |
| 32 | H | 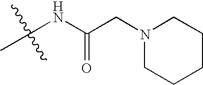 | H | H | H,H | H | Cl | H | H | H |
| 33 | H | 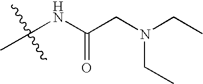 | H | H | H,H | H | Cl | H | H | H |
| 34 | H | 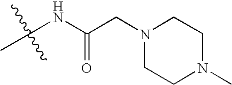 | H | H | H,H | H | Cl | H | H | H |
| 35 | H | NHCO₂C(CH₃)₃ | H | H | H,H | H | H | H | H | H |
| 36 | H | NHCO₂C(CH₃)₃ | H | H | H,H | H | Cl | H | H | H |
| 37 | H | 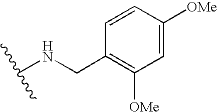 | H | H | H,H | H | H | H | H | H |
| 38 | H | 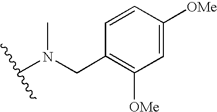 | H | H | H,H | H | H | H | H | —CH₂OH |
| 39 | H | NHCH₃ | H | H | H,H | H | H | H | H | —CH₂OH |
| 40 | H | NMe₂ | H | H | H,H | H | H | H | H | —CH₂OH |
| 41 | H | NHSO₂CH₃ | H | H | H,H | H | Cl | H | H | H |
| 42 | H | NHCOCH═CH₂ | H | H | H,H | H | Cl | H | H | H |

TABLE 4

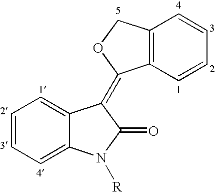

| Example | | | | | | | | | | |
|---|---|---|---|---|---|---|---|---|---|---|
| Number | 1 | 2 | 3 | 4 | 5 | 1′ | 2′ | 3′ | 4′ | R |
| 43 | H | H | NH₂ | H | H,H | H | H | F | H | H |
| 44 | H | H | NH₂ | H | H,H | H | F | H | H | H |
| 45 | H | H | NH(CH₂)₂Cl | H | H,H | H | H | H | H | H |
| 46 | H | H | NH(CH₂)₂Cl | H | H,H | H | H | F | H | H |
| 47 | H | H | NH(CH₂)₂Cl | H | H,H | H | F | H | H | H |
| 48 | H | H | 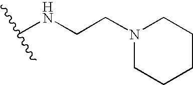 | H | H,H | H | H | H | H | H |
| 49 | H | H | 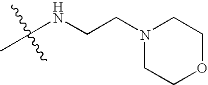 | H | H,H | H | H | H | H | H |
| 50 | H | H | 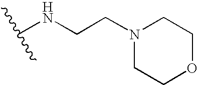 | H | H,H | H | H | F | H | H |
| 51 | H | H | 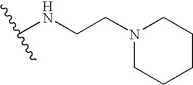 | H | H,H | H | H | F | H | H |
| 52 | H | H | 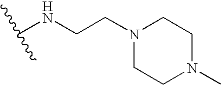 | H | H,H | H | H | H | H | H |
| 53 | H | H | 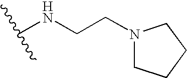 | H | H,H | H | H | H | H | H |
| 54 | H | H | 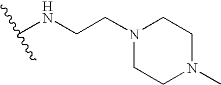 | H | H,H | H | H | F | H | H |
| 55 | H | H | 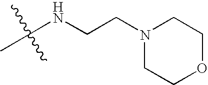 | H | H,H | H | F | H | H | H |
| 56 | H | H | 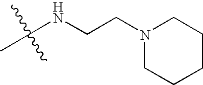 | H | H,H | H | F | H | H | H |
| 57 | H | H | 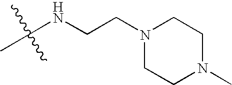 | H | H,H | H | F | H | H | H |
| 58 | H | H | 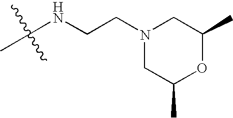 | H | H,H | H | H | H | H | H |
| 59 | H | H | 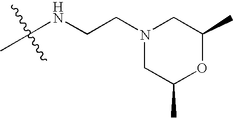 | H | H,H | H | F | H | H | H |
| 60 | H | H | 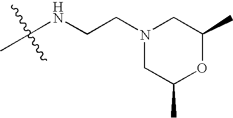 | H | H,H | H | H | F | H | H |
| 61 | H | H | 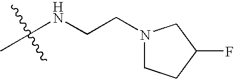 | H | H,H | H | H | H | H | H |
| 62 | H | H | 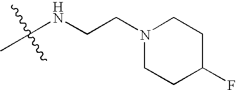 | H | H,H | H | H | H | H | H |
| 63 | H | H | 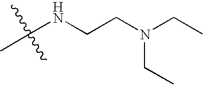 | H | H,H | H | F | H | H | H |
| 64 | H | H | 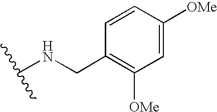 | H | H,H | H | H | H | H | H |
| 65 | H | H | 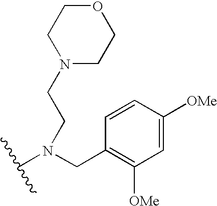 | H | H,H | H | H | H | H | H |
| 66 | H | H | 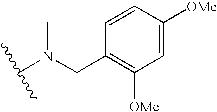 | H | H,H | H | H | F | H | H |
| 67 | H | H | 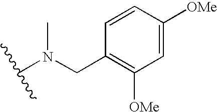 | H | H,H | H | H | H | H | H |
| 68 | H | H | 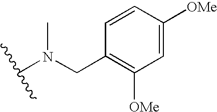 | H | H,H | H | Cl | H | H | H |
| 69 | H | H | 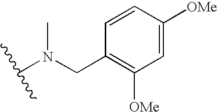 | H | H,H | H | H | H | F | H |
| 70 | H | H | 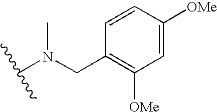 | H | H,H | H | F | H | H | H |
| 71 | H | H | 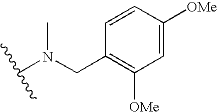 | H | H,H | H | H | Cl | H | H |
| 72 | H | H | NHCH₃ | H | H,H | H | H | F | H | H |
| 73 | H | H | NHCH₃ | H | H,H | H | F | H | H | H |
| 74 | H | H | NHCH₃ | H | H,H | H | Cl | H | H | H |
| 75 | H | H | NHCH₃ | H | H,H | H | H | Cl | H | H |
| 76 | H | H | NHCH₃ | H | H,H | H | H | H | F | H |
| 77 | H | H | N(CH₃)₂ | H | H,H | H | Cl | H | H | H |
| 78 | H | H | NHC(C₆H₅)₃ | H | H,H | H | Cl | H | H | H |
| 79 | H | H | N(CH₂C₆H₅)₂ | H | H,H | H | Cl | H | H | H |
| 80 | H | H | 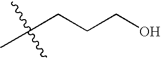 | H | H,H | H | H | H | H | H |
| 81 | H | H | 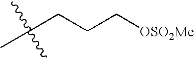 | H | H,H | H | H | H | H | H |
| 82 | H | H | 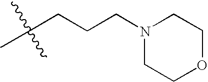 | H | H,H | H | H | H | H | H |
| 83 | H | H | 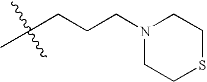 | H | H,H | H | H | H | H | H |
| 84 | H | H | 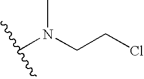 | H | H,H | H | H | H | H | H |
| 85 | H | H | 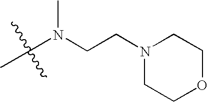 | H | H,H | H | H | H | H | H |
| 86 | H | H | 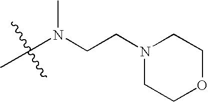 | H | H,H | H | F | H | H | H |
| 87 | H | H | 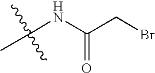 | H | H,H | H | Cl | H | H | H |
| 88 | H | H | 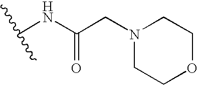 | H | H,H | H | Cl | H | H | H |
| 89 | H | H | 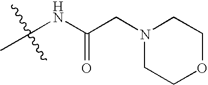 | H | H,H | H | H | H | H | H |
| 90 | H | H | 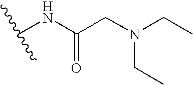 | H | H,H | H | Cl | H | H | H |
| 91 | H | H | 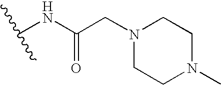 | H | H,H | H | Cl | H | H | H |
| 92 | H | H | 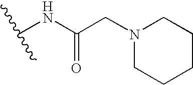 | H | H,H | H | Cl | H | H | H |
| 93 | H | H | NH(CH₂)₂OH | H | H,H | H | H | H | H | H |
| 94 | H | H | NH(CH₂)₂OH | H | H,H | H | H | F | H | H |
| 95 | H | H | NH(CH₂)₂OCOCH₃ | H | H,H | H | H | H | H | H |
| 96 | H | H | NH(CH₂)₂OCOCH₃ | H | H,H | H | H | F | H | H |
| 97 | H | H | 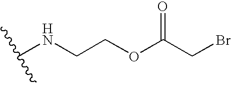 | H | H,H | H | H | H | H | H |
| 98 | H | H | 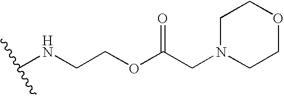 | H | H,H | H | H | H | H | H |
| 99 | H | H | 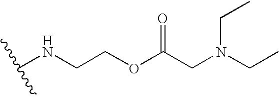 | H | H,H | H | H | H | H | H |
| 100 | H | H | 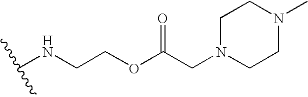 | H | H,H | H | H | H | H | H |
| 101 | H | H | 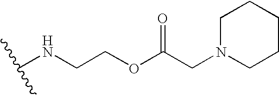 | H | H,H | H | H | H | H | H |
| 102 | H | H | Br | H | H,H | H | H | H | H | H |
| 103 | H | H | 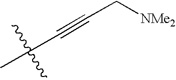 | H | H,H | H | H | H | H | H |
| 104 | H | H | 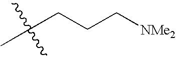 | H | H,H | H | H | H | H | H |
| 135 | H | H | 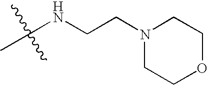 | H | H,H | H | Cl | H | H | H |

TABLE 5

| | | | | | | | | | | 33 |
|---|---|---|---|---|---|---|---|---|---|---|
| 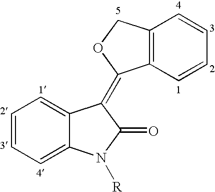 | | | | | | | | | | |
| Example | | | | | | | | | | |
| Number | 1 | 2 | 3 | 4 | 5 | 1′ | 2′ | 3′ | 4′ | R |
| 105 | H | H | H | H | —(CH₂)₂NH(CH₂)₂— | H | H | H | H | H |
| 106 | H | H | H | H | —CH₂COOH | H | H | H | H | H |
| 107 | H | H | H | H | —CH₂COOH | H | Cl | H | H | H |
| 108 | H | H | H | H | —CH₂COOH | H | H | F | H | H |
| 109 | H | H | H | H | —CH₂CH₂OH | H | H | H | H | H |
| 110 | H | H | H | H | —(CH₂)₂OSO₂CH₃ | H | H | H | H | H |
| 111 | H | H | H | H | 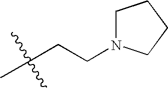 | H | H | H | H | H |
| 112 | H | H | H | H | 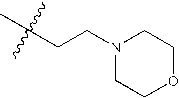 | H | H | H | H | H |
| 113 | H | H | H | H | 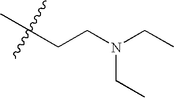 | H | H | H | H | H |
| 114 | H | H | H | H | 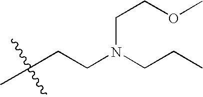 | H | H | H | H | H |
| 115 | H | H | H | H | 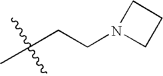 | H | H | H | H | H |
| 116 | H | H | H | H | —CH₂N(CH₃)₂ | H | H | H | H | H |
| 117 | H | H | H | H | —CH₂NCO | H | H | H | H | H |
| 118 | H | H | H | H | —CH₂NHCONH₂ | H | H | H | H | H |
| 119 | H | H | H | H | —CH₂NHCO₂C₂H₅ | H | H | H | H | H |
| 120 | H | H | H | H | 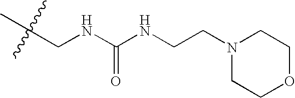 | H | H | H | H | H |
| 121 | H | H | H | H | 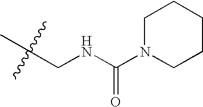 | H | H | H | H | H |
| 122 | H | H | H | H | 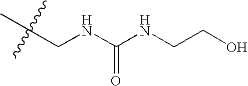 | H | H | H | H | H |
| 123 | H | H | H | H | 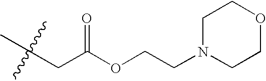 | H | H | H | H | H |
| 124 | H | H | H | H | 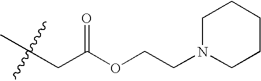 | H | H | F | H | H |
| 125 | H | H | H | H | —CH₂CO₂CH₃ | H | H | F | H | H |
| 126 | H | H | H | H | —COOH | H | H | H | H | H |
| 127 | H | H | H | H | 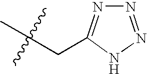 | H | H | H | H | H |
| 128 | H | H | H | H | —CH₂CONH₂ | H | H | H | H | H |
| 129 | H | H | H | H | Me | H | H | H | H | H |
| 130 | H | H | H | H | 2xMe | H | H | H | H | H |
| 131 | H | H | OMe | H | —CH₂COOH | H | H | H | H | H |
| 132 | H | OMe | H | H | —CH₂COOH | H | H | H | H | H |
| 133 | H | H | H | H | —CH₂COONa | H | H | H | H | H |
| 134 | H | H | H | H | —CH₂COONa | H | H | F | H | H |

TABLE 6

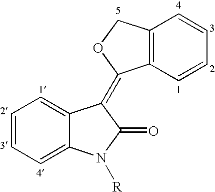

| Example | | | | | | | | | | |
|---|---|---|---|---|---|---|---|---|---|---|
| Number | 1 | 2 | 3 | 4 | 5 | 1′ | 2′ | 3′ | 4′ | R |
| 136 | H | H | 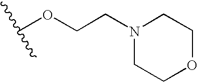 | H | H,H | H | OMe | F | H | H |
| 137 | H | H | 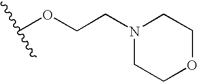 | H | H,H | H | F | H | H | H |
| 138 | H | H | 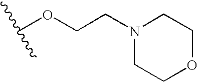 | H | H,H | H | Cl | H | H | H |
| 139 | H | H | 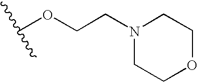 | H | H,H | H | 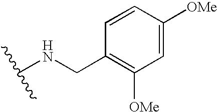 | F | H | H |

### Example 28

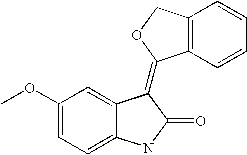

### 3-(3H-Isobenzofuran-1-ylidene)-5-methoxy-1,3-dihydro-indol-2-one

To a stirred solution of 5-methoxyoxindole (100 mg, 0.61 mmol) in anhydrous THF (5 ml) under nitrogen was added 1.0M LiHMDS/THF solution (1.3 ml, 1.3 mmol). The mixture was stirred at room temperature for 10 minutes, and phthalide (74 mg, 0.55 mmol) was added. The mixture was stirred at room temperature for 5 hours and was then poured into a mixture of THF (10 ml) and 2M HCl (10 ml). The mixture was heated at 45° C. for 30 minutes, cooled to room temperature, and poured into water (125 ml). The resulting solid was separated, washed with water and dried under vacuum to give 3-(3H-Isobenzofuran-1-ylidene)-5-methoxy-1,3-dihydro-indol-2-one (70 mg, 46%).

¹H NMR (500 MHz, DMSO-d₆) δ ppm 3.69 (s, 3H) 5.76 (s, 2H) 6.67 (m, 2H) 7.41 (s, 1H) 7.51 (m, 1H) 7.61 (m, 2H) 9.62 (d, J=8.30 Hz, 1H) 10.19 (s, 1H)

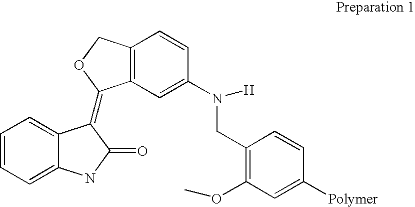

### 3-[6-(2-Morpholin-4-yl-ethylamino)-3-isobenzofuran-1-ylidene]-1,3-dihydro-indol-2-one-6-bonding to 4-formyl-3-methoxyphenoxymethyl resin

To a mixture of 3-(6-amino-3H-isobenzofuran-1-ylidene)-1,3-dihydro-indol-2-one (0.5 g, 1.89 mmol), 4-formyl-3-methoxyphenoxymethyl resin (1.1 g, 1.26 mmole) in 1% AcOH/DMF (21 ml) was added sodium triacetoxyborohydride (2.7 g, 12.6 mmol). The resulting mixture was gently stirred at room temperature for 48 hours. The resin was separated, and washed with DMF, MeOH, and CHCl₃, alternately. Removal of the solvent provided 3-[6-(2-morpholin-4-yl-ethylamino)-3-isobenzofuran-1-ylidene]-1,3-dihydro-indol-2-one-6-bound to 4-formyl-3-methoxyphenoxymethyl resin (1.1 g).

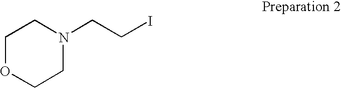

### Preparation of 4-(2-Iodo-ethyl)-morpholine

A mixture of 4-(2-chloro-ethyl)-morpholine hydrochloride (5 g, 26.9 mmole), and sodium iodide (20 g, 134.4 mmole) in acetone (50 ml) was refluxed for 16 hours. After cooled to room temperature, the reaction was partitioned between CHCl₃ and brine. The aqueous layer was extracted with CHCl₃ (2×25 ml). The combined organic layers were washed with brine, and dried over anhydrous Mg₂SO4. Removal of the solvent gave 4-(2-iodo-ethyl)-morpholine as a pale yellowish oil (3.84 g, 59%).

¹H NMR (500 MHz, CDCl₃) δ ppm 2.51 (brs, 4H) 2.74 (t, J=7.81 Hz, 2H) 3.22 (t, J=7.81 Hz, 2H) 3.73 (t, J=4.39 Hz, 4H)

### Example 29

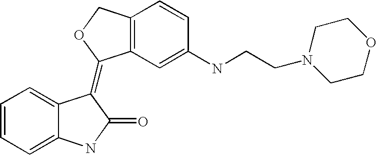

### 3-[6-(2-Morpholin-4-yl-ethylamino)-3-isobenzofuran-1-ylidene]-1,3-dihydro-indol-2-one

A mixture of 3-[6-(2-morpholin-4-yl-ethylamino)-3-isobenzofuran-1-ylidene]-1,3-dihydro-indol-2-one-6-bonding to 4-formyl-3-methoxyphenoxymethyl resin (200 mg, 0.182 mmol), 4-(2-iodo-ethyl)-morpholine (660 mg, 2.72 mmol), N,N-diisopropylethylamine (0.94 ml, 5.44 mmol) in 1,4-dioxane was heated at 106° C. under nitrogen for 16 hours. The resulting resin was separated, and washed with DMF, MeOH, and CHCl₃, alternately. The washed resin was then mixed with 10% trifluoroacetic acid in CH₂Cl₂ (5 ml), and stirred at room temperature for 1 hour. The resulting mixture was poured into a mixture of CHCl₃, and saturated aqueous NaHCO₃ solution. The aqueous layer was extracted with CHCl₃. The combined organic layers were washed with brine, dried over anhydrous MgSO₄. Purification of the mixture by preparative silica gel TLC, eluted with 9:1 CHCl₃/MeOH, led to 3-[6-(2-Morpholin-4-yl-ethylamino)-3-isobenzofuran-1-ylidene]-1,3-dihydro-indol-2-one as a yellow solid (32 mg, 14%).

¹H NMR (500 MHz, CDCl₃) δ ppm 2.44 (br s, 4H) 2.62 (t, J=5.61 Hz, 2H) 3.25 (t, J=5.86 Hz, 2H) 3.68 (t, J=4.64 Hz, 4H) 5.53 (s, 2H) 6.80 (m, 2H) 6.98 (td, J=7.69, 1.22 Hz, 1H) 7.08 (td, J=7.57, 1.46 Hz, 1H) 7.18 (d, J=8.30 Hz, 1H) 7.47 (s, 1H) 7.92 (d, J=7.81 Hz, 1H) 9.03 (d, J=2.44 Hz, 1H)

### Example 30

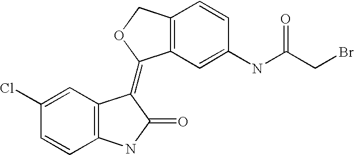

### 2-Bromo-N-[3-(5-chloro-2-oxo-1,2-dihydro-indol-3-ylidene)-1,3-dihydro-isobenzofuran-5-yl]-acetamide

To a stirred suspension of 3-(6-amino-3H-isobenzofuran-1-ylidene)-5-chloro-1,3-dihydro-indol-2-one (300 mg, 1.0 mmol) in THF (15 ml), was added bromoacetic anhydride (311 mg, 1.2 mmol). The mixture was stirred for 1 hour and was poured into water (150 ml). The solid was filtered, washed with water and dried under vacuum to give 2-bromo-N-[3-(5-chloro-2-oxo-1,2-dihydro-indol-3-ylidene)-1,3-dihydro-isobenzofuran-5-yl]-acetamide as a yellow solid (396 mg, 94%).

¹H NMR (500 MHz, DMSO-d₆) δ ppm 4.10 (s, 2H) 5.81 (s, 2H) 6.84 (d, J=8.30 Hz, 1H) 7.16 (dd, J=8.06, 2.20 Hz, 1H) 7.65 (d, J=8.30 Hz, 1H) 7.81 (d, J=1.95 Hz, 1H) 8.14 (d, J=8.30 Hz, 1H) 9.64 (s, 1H) 10.57 (s, 1H) 10.75 (s, 1H).

### Example 31

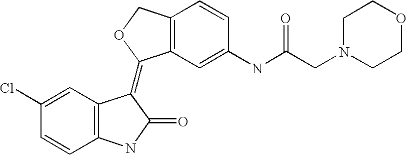

### N-[3-(5-Chloro-2-oxo-1,2-dihydro-indol-3-ylidene)-1,3-dihydro-isobenzofuran-5-yl]-2-morpholin-4-yl-acetamide

A mixture of 2-bromo-N-[3-(5-chloro-2-oxo-1,2-dihydro-indol-3-ylidene)-1,3-dihydro-isobenzofuran-5-yl]-acetamide (63 mg, 0.15 mmol) in morpholine (1 ml) was stirred at 40° C. under nitrogen for 40 minutes. The mixture was poured into water (75 ml). The solid was filtered, washed with water and dried under vacuum to give N-[3-(5-chloro-2-oxo-1,2-dihydro-indol-3-ylidene)-1,3-dihydro-isobenzofuran-5-yl]-2-morpholin-4-yl-acetamide as a yellow solid (60 mg, 94%).

¹H NMR (300 MHz, DMSO-d₆) δ ppm 2.55 (m, 4H) 3.19 (s, 2H) 3.65 (m, 4H) 5.81 (s, 2H) 6.84 (d, J=8.21 Hz, 1H) 7.16 (dd, J=8.21, 2.35 Hz, 1H) 7.62 (d, J=8.21 Hz, 1H) 7.81 (d, J=2.05 Hz, 1H) 8.06 (dd, J=8.21, 1.76 Hz, 1H) 9.66 (d, J=1.76 Hz, 1H) 10.07 (s, 1H) 10.53 (s, 1H).

The following Example 32 through 34 were prepared using the experiment procedure described in Example 31, but with the appropriate reagent, reaction conditions and reactant substitutions that will be readily realized by those of ordinary skill in this art without the exercise of undue experimentation

### Example 32

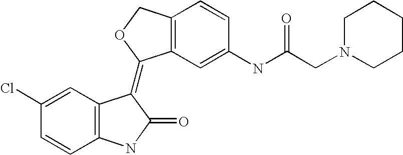

### N-[3-(5-Chloro-2-oxo-1,2-dihydro-indol-3-ylidene)-1,3-dihydro-isobenzofuran-5-yl]-2-piperidin-1-yl-acetamide

¹H NMR (300 MHz, DMSO-d₆) δ ppm 1.41 (brs, 2H) 1.58 (m, 4H) 2.47 (brs, 4H) 3.12 (s, 2H) 5.81 (s, 2H) 6.84 (d, J=8.21 Hz, 1H) 7.16 (dd, J=8.21, 2.35 Hz, 1H) 7.62 (d, J=8.21 Hz, 1H) 7.81 (d, J=2.05 Hz, 1H) 8.08 (dd, J=8.21, 1.76 Hz, 1H) 9.65 (d, J=1.76 Hz, 1H) 9.96 (s, 1H).

### Example 33

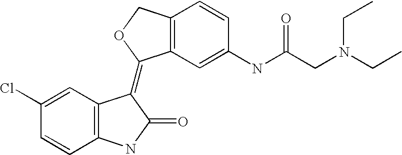

### N-[3-(5-Chloro-2-oxo-1,2-dihydro-indol-3-ylidene)-1,3-dihydro-isobenzofuran-5-yl]-2-diethylamino-acetamide

¹H NMR (300 MHz, DMSO-d₆) δ ppm 1.03 (t, J=7.04 Hz, 6H) 2.62 (q, J=7.23 Hz, 4H) 3.19 (s, 2H) 5.79 (s, 2H) 6.82 (d, J=8.21 Hz, 1H) 7.14 (dd, J=8.36, 2.20 Hz, 1H) 7.61 (d, J=8.21 Hz, 1H) 7.79 (d, J=2.05 Hz, 1H) 8.06 (dd, J=8.35, 1.61 Hz, 1H) 9.63 (d, J=1.47 Hz, 1H) 9.88 (s, 1H) 10.50 (s, 1H)

### Example 34

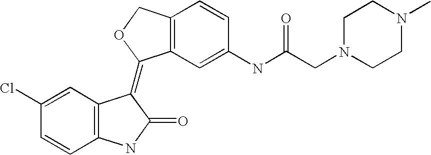

### N-[3-(5-Chloro-2-oxo-1,2-dihydro-indol-3-ylidene)-1,3-dihydro-isobenzofuran-5-yl]-2-(4-methyl-piperazin-1-yl)-acetamide

¹H NMR (300 MHz, DMSO-d₆) δ ppm 2.18 (s, 3H) 2.39 (brs, 4H) 2.55 (brs, 4H) 3.16 (s, 2H) 5.80 (s, 2H) 6.84 (d, J=8.21 Hz, 1H) 7.16 (dd, J=8.21, 2.05 Hz, 1H) 7.62 (d, J=8.21 Hz, 1H) 7.80 (d, J=2.05 Hz, 1H) 8.08 (dd, J=8.50, 1.76 Hz, 1H) 9.64 (d, J=1.76 Hz, 1H) 9.99 (s, 1H) 10.54 (s, 1H).

### Example 35

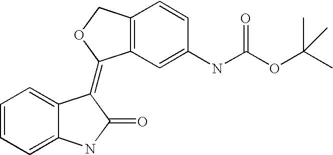

### [3-(2-Oxo-1,2-dihydro-indol-3-ylidene)-1,3-dihydro-isobenzofuran-5-yl]-carbamic acid t-butyl ester

To a stirred suspension of 3-(6-amino-3H-isobenzofuran-1-ylidene)-1,3-dihydro-indol-2-one (500 mg, 1.89 mmol) and triethylamine (574 mg, 5.67 mmol) in MeOH (30 ml), was added 1M di-tert-butyl dicarbonate solution in THF (3.8 ml, 3.8 mmol). The mixture was heated at 60° C. for 4 hours, and then cooled to room temperature. After stored in refrigerator, the solid was separated, washed with MeOH and dried under vacuum to give [3-(2-oxo-1,2-dihydro-indol-3-ylidene)-1,3-dihydro-isobenzofuran-5-yl]-carbamic acid t-butyl ester as a yellow solid (250 mg, 36%).

¹H NMR (500 MHz, DMSO-d₆) δ ppm 1.50 (s, 9H) 5.73 (s, 2H) 6.83 (d, J=7.81 Hz, 1H) 6.95 (t, J=7.57 Hz, 1H) 7.11 (t, J=7.57 Hz, 1H) 7.52 (d, J=8.30 Hz, 1H) 7.69 (d, J=7.81 Hz, 1H) 7.83 (d, J=7.81 Hz, 1H) 9.59 (s, 1H) 9.69 (s, 1H) 10.33 (s, 1H); LR MS (EI): 364 (M⁺).

The following Example 36 was prepared using the experiment procedure described in Example 35, but with the appropriate reagent, reaction conditions and reactant substitutions that will be readily realized by those of ordinary skill in this art without the exercise of undue experimentation

### Example 36

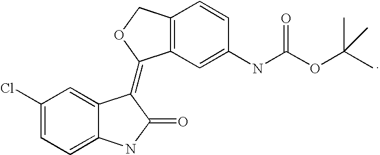

### [3-(5-Chloro-2-oxo-1,2-dihydro-indol-3-ylidene)-1,3-dihydro-isobenzofuran-5-yl]-carbamic acid t-butyl ester

¹H NMR (500 MHz, DMSO-d₆) δ ppm 1.50 (s, 9H) 5.77 (s, 2H) 6.83 (d, J=8.30 Hz, 1H) 7.15 (dd, J=8.30, 2.44 Hz, 1H) 7.55 (d, J=8.30 Hz, 1H) 7.71 (d, J=7.81 Hz, 1H) 7.80 (d, J=2.44 Hz, 1H) 9.62 (s, 1H) 9.68 (s, 1H) 10.48 (s, 1H); LR MS (EI): 398 (M⁺) 400 (M+2).

### Example 37

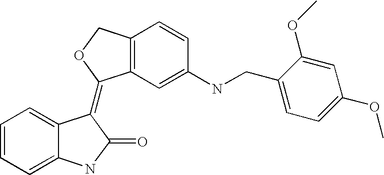

### 3-[6-(2,4-Dimethoxy-benzylamino)-3H-isobenzofuran-1-ylidene]-1,3-dihydro-indol-2-one

To a stirred solution of 3-(6-amino-3H-isobenzofuran-1-ylidene)-1,3-dihydro-indol-2-one (1 g, 3.79 mmol) and 2,4-dimethoxybenzaldehyde (0.75 g, 4.54 mmol) in 1% AcOH/DMF (30 ml), was added sodium triacetoxyborohydride (3.2 g, 15.1 mmol). The mixture was stirred at room temperature for 16 hours, diluted with CHCl₃ (200 ml), washed with saturated aqueous NaHCO₃ solution (200 ml) and water (2×200 ml), dried over anhydrous Na₂SO₄. Removal of the solvent provided the crude product. Recrystallization of the crude product from MeOH resulted in 3-[6-(2,4-dimethoxy-benzylamino)-3H-isobenzofuran-1-ylidene]-1,3-dihydro-indol-2-one as a bright yellow solid (1.3 g, 83%).

¹H NMR (500 MHz, DMSO-d₆) δ ppm 3.73 (s, 3H) 3.83 (s, 3H) 4.21 (d, J=5.86 Hz, 2H) 5.62 (s, 2H) 6.23 (t, J=5.86 Hz, 1H) 6.44 (dd, J=8.54, 2.20 Hz, 1H) 6.57 (d, J=2.44 Hz, 1H) 6.81 (d, J=7.32 Hz, 1H) 6.91 (m, 2H) 7.08 (m, 1H) 7.23 (d, J=8.30 Hz, 1H) 7.29 (d, J=8.30 Hz, 1H) 7.82 (d, J=7.81 Hz, 1H) 9.00 (d, J=2.44 Hz, 1H) 10.30 (s, 1H); LR MS (EI): 414 (M⁺).

### Example 38

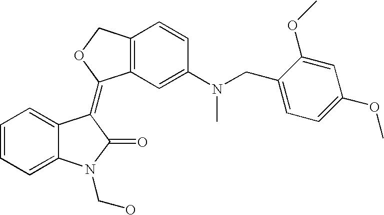

### 3-{6-[(2,4-Dimethoxy-benzyl)-methyl-amino]-3H-isobenzofuran-1-ylidene}-1-hydroxymethyl-1,3-dihydro-indol-2-one

To a stirred suspension of 3-[6-(2,4-dimethoxy-benzylamino)-3H-isobenzofuran-1-ylidene]-1,3-dihydro-indol-2-one (1 g, 2.41 mmol) and 37% formaldehyde aqueous solution (2 ml, 27 mmol) in acetonitrile (30 ml), was added sodium cyanoborohydride (606 mg, 9.64 mmol). The mixture was stirred at room temperature for 2 hours, and 10% AcOH aqueous solution (30 ml) was added. The mixture was continuously stirred for another 10 minutes. The yellow precipitate separated, washed with MeOH and dried under vacuum to give a crude product. Recrystallization of the crude product with MeOH led to 3-{6-[(2,4-dimethoxy-benzyl)-methyl-amino]-3H-isobenzofuran-1-ylidene}-1-hydroxymethyl-1,3-dihydro-indol-2-one as yellow needles (0.9 g, 82%).

¹H NMR (500 MHz, DMSO-d₆) δ ppm 3.06 (s, 3H) 3.72 (s, 3H) 3.82 (s, 3H) 4.53 (s, 2H) 5.20 (d, J=6.83 Hz, 2H) 5.70 (s, 2H) 6.15 (t, J=7.32 Hz, 1H) 6.42 (dd, J=8.54, 2.20 Hz, 1H) 6.59 (d, J=2.44 Hz, 1H) 6.90 (d, J=8.30 Hz, 1H) 7.00 (dd, J=8.30, 2.44 Hz, 1H) 7.04 (m, 1H) 7.09 (d, J=7.81 Hz, 1H) 7.19 (m, 1H) 7.40 (d, J=8.30 Hz, 1H) 7.91 (d, J=7.81 Hz, 1H) 9.27 (d, J=2.44 Hz, 1H); LR MS (FAB+): 459 (M+1).

### Example 39

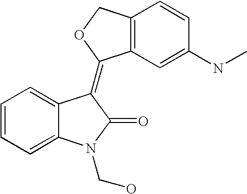

### 1-Hydroxymethyl-3-(6-methylamino-3H-isobenzofuran-1-ylidene)-1,3-dihydro-indol-2-one

A solution of 3-{6-[(2,4-dimethoxy-benzyl)-methyl-amino]-3H-isobenzofuran-1-ylidene}-1-hydroxymethyl-1,3-dihydro-indol-2-one (600 mg, 1.31 mmol) in a mixture of THF (20 ml) and 2M HCl aqueous solution (20 ml) was heated at 50° C. for 16 hours. The mixture was concentrated, and then partitioned between CHCl₃ (200 ml) and saturated NaHCO₃ solution (200 ml). The aqueous layer was extracted with CHCl₃ (2×100 ml). The combined organic layers were washed with water (100 ml), dried over anhydrous Na₂SO₄, and evaporated to give a dark brown oil as a crude product. Purification of the crude product by silica gel column chromatography, eluted with a gradient of MeOH in EtOAc, yielded 1-hydroxymethyl-3-(6-methylamino-3H-isobenzofuran-1-ylidene)-1,3-dihydro-indol-2-one as a yellow solid (25 mg, 6%).

¹H NMR (500 MHz, DMSO-d₆) δ ppm 2.77 (d, J=4.88 Hz, 3H) 5.21 (d, J=6.83 Hz, 2H) 5.68 (s, 2H) 6.04 (q, J=4.72 Hz, 1H) 6.15 (t, J=6.83 Hz, 1H) 6.92 (dd, J=8.30, 2.44 Hz, 1H) 7.04 (t, J=7.57 Hz, 1H) 7.09 (d, J=7.32 Hz, 1H) 7.19 (t, J=7.08 Hz, 1H) 7.35 (d, J=8.30 Hz, 1H) 7.91 (d, J=7.32 Hz, 1H) 8.95 (d, J=2.44 Hz, 1H); LR MS (EI): 308 (M⁺).

### Example 40

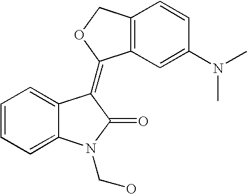

### 3-(6-Dimethylamino-3H-isobenzofuran-1-ylidene)-1-hydroxymethyl-1,3-dihydro-indol-2-one

To a suspension of 3-(6-amino-3H-isobenzofuran-1-ylidene)-1,3-dihydro-indol-2-one (100 mg, 0.378 mmole) were added saturated formaldehyde aqueous solution, and sodium cyanoborohydride (300 mg, 4.77 mmole). The resulting mixture was stirred at room temperature for 1 hour, and then was acidified with 10% AcOH aqueous solution. After stirred for another 10 minutes, the reaction was partitioned between CHCl₃ and saturated NaHCO₃ aqueous solution. The aqueous layer was extracted with CHCl₃ (2×5 ml). The combined organic layers were washed with brine, and then dried over anhydrous MgSO₄. Removal of the solvent afforded a crude product. Recrystallization of the crude product from MeOH led to 3-(6-dimethylamino-3H-isobenzofuran-1-ylidene)-1-hydroxymethyl-1,3-dihydro-indol-2-one as a yellow solid (90 mg, 74%).

¹H NMR (500 MHz, CDCl₃) δ ppm 3.06 (s, 6H) 5.41 (s, 2H) 5.59 (s, 2H) 6.98 (brs, 1H) 7.04 (d, J=7.81 Hz, 1H) 7.07 (td, J=7.69, 1.22 Hz, 1H) 7.19 (td, J=7.69, 1.22 Hz, 1H) 7.27 (d, J=8.30 Hz, 1H) 7.99 (d, J=7.32 Hz, 1H) 9.22 (s, 1H).

### Example 41

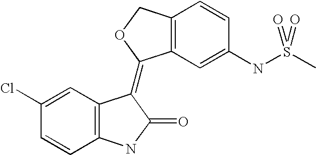

### N-[3-(5-Chloro-2-oxo-1,2-dihydro-indol-3-ylidene)-1,3-dihydro-isobenzofuran-5-yl]-methanesulfonamide

A mixture of 3-(6-amino-3H-isobenzofuran-1-ylidene)-5-chloro-1,3-dihydro-indol-2-one (100 mg, 0.33 mmol), pyridine (1 ml) and methanesulfonyl chloride (76 mg, 0.66 mmol) in THF (3.5 ml) was stirred for 16 hours, and was then poured into water (100 ml). The solid was filtered, washed with water and dried under vacuum to give a crude product. The crude product was triturated with MeOH/water to provide N-[3-(5-chloro-2-oxo-1,2-dihydro-indol-3-ylidene)-1,3-dihydro-isobenzofuran-5-yl]-methanesulfonamide as a yellow solid (100 mg, 81%).

¹H NMR (500 MHz, DMSO-d₆) δ ppm 3.09 (s, 3H) 5.80 (s, 2H) 6.84 (d, J=8.30 Hz, 1H) 7.16 (m, 1H) 7.49 (dd, J=8.30, 1.95 Hz, 1H) 7.64 (d, J=8.30 Hz, 1H) 7.80 (d, J=1.95 Hz, 1H) 9.56 (d, J=1.95 Hz, 1H) 10.08 (s, 1H) 10.53 (s, 1H).

### Example 42

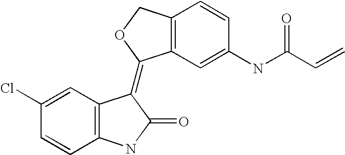

### N-[3-(S-Chloro-2-oxo-1,2-dihydro-indol-3-ylidene)-1,3-dihydro-isobenzofuran-5-yl]-acrylamide

To a stirred mixture of 3-(6-amino-3H-isobenzofuran-1-ylidene)-5-chloro-1,3-dihydro-indol-2-one (300 mg, 11.0 mmol) and triethylamine (0.41 ml, 3.0 mmol) in THF (10 ml), was added 3-bromopropionyl chloride (0.12 ml, 1.2 mmol). The mixture was heated at 45° C. for 2 hours, cooled to room temperature and poured into water (150 ml). The solid was filtered, washed with water and dried under vacuum to give crude product. The crude product was triturated with toluene to afforded N-[3-(5-chloro-2-oxo-1,2-dihydro-indol-3-ylidene)-1,3-dihydro-isobenzofuran-5-yl]-acrylamide as a bright yellow solid (315 mg, 89%).

¹H NMR (500 MHz, DMSO-d₆) δ ppm 5.79 (m, 3H) 6.30 (dd, J=17.09, 1.95 Hz, 1H) 6.54 (dd, J=16.84, 10.01 Hz, 1H) 6.84 (d, J=8.30 Hz, 1H) 7.16 (dd, J=8.30, 2.44 Hz, 1H) 7.64 (d, J=8.79 Hz, 1H) 7.81 (d, J=1.95 Hz, 1H) 8.21 (d, J=8.30 Hz, 1H) 9.68 (s, 1H) 10.53 (d, J=21.48 Hz, 2H).

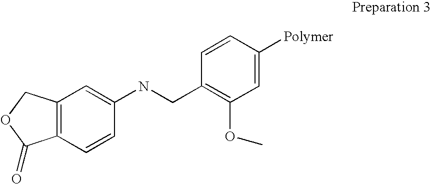

### Preparation of 5-Aminophthalide-5-bound to 4-formyl-3-methoxyphenoxymethyl resin

To a mixture of 5-aminophthalide (5.0 g, 33.5 mmol), 4-formyl-3-methoxyphenoxymethyl resin (6.1 g, 6.71 mmole) in 1% AcOH/DMF (100 ml) was added sodium triacetoxyborohydride (21.3 g, 100 mmol). The resulting mixture was gently stirred at room temperature for 48 hours. The resin was separated, and washed with DMF, MeOH, and CHCl₃, alternately. Removal of the solvent afforded 5-aminophthalide-5-bound to 4-formyl-3-methoxyphenoxymethyl resin (7.0 g).

### Example 43

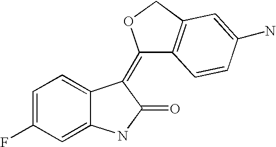

### 3-(5-Amino-3H-isobenzofuran-1-ylidene)-6-fluoro-1,3-dihydro-indol-2-one

A solution of 6-fluorooxindole (280 mg, 1.85 mmole), and 1M LiHMDS/THF (15 ml, 14 mmole) was shaken at room temperature for 5 minutes, followed by addition of 5-aminophthalide-5-bound to 4-formyl-3-methoxyphenoxymethyl resin (1000 mg). The resulting mixture was shaken at room temperature for 16 hours. The resin was separated, and washed with DMF, MeOH, and CHCl₃, alternately, to give 3-(5-amino-3H-isobenzofuran-1-ylidene)-6-fluoro-1,3-dihydro-indol-2-one-5-bonding to 4-formyl-3-methoxyphenoxymethyl resin.

The above washed resin was mixed with 10% trifluoroacetic acid in CH₂Cl₂ (5 ml), and stirred at room temperature for 0.5 hours. The resin residue was separated, and rinsed with CHCl₃. Evaporation of the combined filtrates resulted in a foam, which was recrytallized from CHCl₃/MeOH to yield the trifluoroacetate salt of 3-(5-amino-3H-isobenzofuran-1-ylidene)-6-fluoro-1,3-dihydro-indol-2-one (19 mg, 6%) as a yellow solid.

¹H NMR (500 MHz, DMSO-d₆) δ ppm 5.58 (s, 2H) 6.57 (dd, J=9.28, 2.44 Hz, 1H) 6.67 (m, 3H) 7.68 (dd, J=8.30, 5.86 Hz, 1H) 9.26 (d, J=8.79 Hz, 1H) 10.34 (s, 1H).

The following Example 44 was prepared using the experiment procedure described in Example 43, but with the appropriate reagent, reaction conditions and reactant substitutions that will be readily realized by those of ordinary skill in this art without the exercise of undue experimentation.

### Example 44

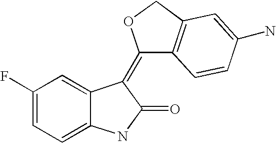

### 3-(5-Amino-3H-isobenzofuran-1-ylidene)-5-fluoro-1,3-dihydro-indol-2-one

¹H NMR (500 MHz, DMSO-d6) δ ppm 5.61 (s, 2H) 6.65 (m, 2H) 6.71 (dd, J=8.30, 4.88 Hz, 1H) 6.79 (m, 1H) 7.46 (dd, J=10.01, 2.68 Hz, 1H) 9.30 (d, J=9.28 Hz, 1H).

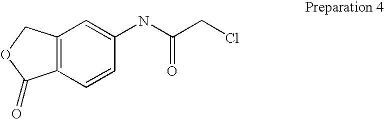

### Preparation of 2-Chloro-N-(1-oxo-1,3-dihydro-isobenzofuran-5-yl)-acetamide

To a stirred solution of chloroacetic anhydride (7.0 g, 41 mmol) in THF (20 ml) was added 5-aminophthalide (3.0 g, 20 mmol). The mixture was stirred at 40° C. for 2 hours, cooled to room temperature and poured into water (100 ml) with stirring. The solid was filtered, washed with water, and dried under vacuum to give 2-chloro-N-(1-oxo-1,3-dihydro-isobenzofuran-5-yl)-acetamide as a light brown powder (4.0 g, 89%).

¹H NMR (500 MHz, DMSO-d₆) δ ppm 4.33 (s, 2H) 5.38 (s, 2H) 7.65 (d, J=8.30 Hz, 1H) 7.82 (d, J=8.30 Hz, 1H) 8.02 (s, 1H) 10.79 (s, 1H).

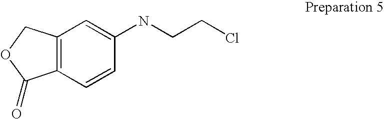

### Preparation of 5-(2-Chloro-ethylamino)-3H-isobenzofuran-1-one

To a suspension of 2-chloro-N-(1-oxo-1,3-dihydroisobenzofuran-5-yl)-acetamide (1.0 g, 4.43 mmol) in THF (15 ml) was added 2M borane-methyl sulfide complex solution in THF (6.6 ml, 13.2 mmol) under nitrogen. After stirred at 60° C. under nitrogen for 2 hours, the resulting mixture was cooled in an ice bath, followed by the addition of aqueous HCl solution. The mixture was stirred at room temperature for 20 minutes, then heated at 60° C. for 40 minutes. After cooled to room temperature, the mixture was basified with aqueous NaOH solution, and then it was partitioned between water (50 ml) and CHCl₃ (50 ml). The aqueous layer was extracted with CHCl₃ (2×50 ml). The combined organic layers were washed with water, dried over anhydrous Na₂SO₄. Removal of the solvent led to a crude product. Trituration of the crude product with MeOH gave 5-(2-chloro-ethylamino)-3H-isobenzofuran-1-one as a light brown solid (0.4 g, 42%).

¹H NMR (500 MHz, DMSO-d₆) δ ppm 3.51 (q, J=6.35 Hz, 2H) 3.75 (t, J=6.10 Hz, 2H) 5.20 (s, 2H) 6.70 (s, 1H) 6.78 (dd, J=8.30, 1.95 Hz, 1H) 7.05 (t, J=5.86 Hz, 1H) 7.50 (d, J=8.30 Hz, 1H).

### Example 45

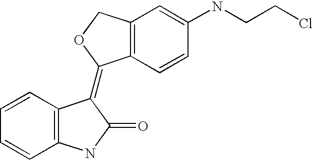

### 3-[5-(2-Chloro-ethylamino)-3H-isobenzofuran-1-ylidene]-1,3-dihydro-indol-2-one

To a stirred solution of oxindole (707 mg, 5.3 mmol) in anhydrous dimethoxyethane (20 ml) under nitrogen was added 1M LiHMDS/THF solution (18.5 ml, 18.5 mmol). The mixture was stirred at room temperature for 10 minutes, and 5-(2-chloro-ethylamino)-3H-isobenzofuran-1-one (900 mg, 4.25 mmol) was added. The mixture was stirred at room temperature for 2.5 hours and poured into 0.1M HCl solution (400 ml). The mixture was stirred for 30 min, then basified with aqueous NaOH solution. The precipitants were filtered, washed with water, and dried under vacuum to result in a crude product mixture. The crude product mixture was purified by silica gel column chromatography, eluted with a gradient of MeOH in CHCl₃. The major product, 3-[5-(2-chloro-ethylamino)-3H-isobenzofuran-1-ylidene]-1,3-dihydro-indol-2-one (Example 45), was obtained as a yellow solid (560 mg, 40%) and so was the minor product, 3-[5-(2-hydroxy-ethylamino)-3H-isobenzofuran-1-ylidene]-1,3-dihydro-indol-2-one (Example 93), as a yellow solid.

Example 45: ¹H NMR (500 MHz, DMSO-d₆) δ ppm 3.55 (q, J=6.35 Hz, 2H) 3.77 (t, J=6.10 Hz, 2H) 5.64 (s, 2H) 6.78 (m, 3H) 6.90 (t, J=7.08 Hz, 1H) 7.03 (m, 2H) 7.75 (d, J=7.32 Hz, 1H) 9.39 (d, J=8.79 Hz, 1H) 10.22 (s, 1H).

The following Example 46 through 47 were prepared using the experiment procedure described in Example 45, but with the appropriate reagent, reaction conditions and reactant substitutions that will be readily realized by those of ordinary skill in this art without the exercise of undue experimentation.

### Example 46

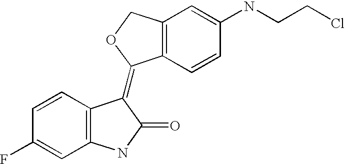

### 3-[5-(2-Chloro-ethylamino)-3H-isobenzofuran-1-ylidene]-6-fluoro-1,3-dihydro-indol-2-one

¹H NMR (500 MHz, DMSO-d₆) δ ppm 3.54 (q, J=5.86 Hz, 2H) 3.77 (t, J=6.10 Hz, 2H) 5.64 (s, 2H) 6.59 (dd, J=9.28, 2.44 Hz, 1H) 6.71 (m, 1H) 6.79 (m, 2H) 7.07 (t, J=5.86 Hz, 1H) 7.71 (dd, J=8.79, 5.86 Hz, 1H) 9.33 (d, J=8.79 Hz, 1H) 10.37 (s, 1H).

### Example 47

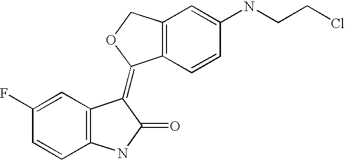

### 3-[5-(2-Chloro-ethylamino)-3H-isobenzofuran-1-ylidene]-5-fluoro-1,3-dihydro-indol-2-one

¹H NMR (500 MHz, DMSO-d₆) δ ppm 3.55 (q, J=5.86 Hz, 2H) 3.77 (t, J=6.10 Hz, 2H) 5.66 (s, 2H) 6.74 (dd, J=8.30, 4.88 Hz, 1H) 6.82 (m, 3H) 7.13 (br, 1H) 7.48 (dd, J=10.01, 2.69 Hz, 1H) 9.36 (d, J=9.28 Hz, 1H) 10.23 (s, 1H).

### Example 48

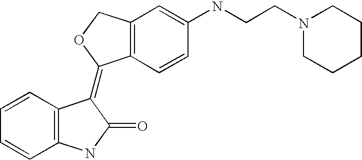

### 3-[5-(2-Piperidin-1-yl-ethylamino)-3H-isobenzofuran-1-ylidene]-1,3-dihydro-indol-2-one

A mixture of 3-[5-(2-chloro-ethylamino)-3H-isobenzofuran-1-ylidene]-1,3-dihydro-indol-2-one (1.10 g, 3.37 mmol) and piperidine (8 ml, 80.9 mmol) was heated at 110° C. for 4 hours. After cooled to room temperature, the mixture was poured into an ice water (150 ml) with stirring. The solid was filtered, washed with water and dried to give a crude product. Trituration of the crude product with CHCl₃/hexanes afforded 3-[5-(2-piperidin-1-yl-ethylamino)-3H-isobenzofuran-1-ylidene]-1,3-dihydro-indol-2-one as a yellow solid (1.13 g, 89%).

¹H NMR (500 MHz, DMSO-d₆) δ ppm 1.39 (m, 2H) 1.51 (m, 4H) 2.39 (br, 4H) 2.48 (m, 2H) 3.24 (q, J=5.86 Hz, 2H) 5.62 (s, 2H) 6.65 (t, J=5.37 Hz, 1H) 6.70 (s, 1H) 6.74 (dd, J=8.79, 1.95 Hz, 1H) 6.78 (d, J=7.32 Hz, 1H) 6.89 (t, J=7.08 Hz, 1H) 7.00 (td, J=7.69, 1.22 Hz, 1H) 7.74 (d, J=7.81 Hz, 1H) 9.36 (d, J=8.79 Hz, 1H) 10.20 (s, 1H).

The following Example 49 through 63 were prepared using the experiment procedure described in Example 48, but with the appropriate reagent, reaction conditions and reactant substitutions that will be readily realized by those of ordinary skill in this art without the exercise of undue experimentation.

### Example 49

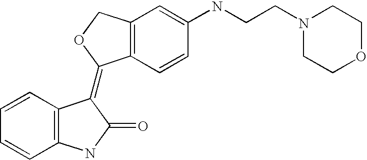

### 3-[5-(2-Morpholin-4-yl-ethylamino)-3H-isobenzofuran-1-ylidene]-1,3-dihydro-indol-2-one

¹H NMR (500 MHz, DMSO-d₆) δ ppm 2.44 (brs, 4H) 2.53 (t, J=6.83 Hz, 2H) 3.27 (q, J=6.35 Hz, 2H) 3.60 (t, J=4.39 Hz, 4H) 5.63 (s, 2H) 6.70 (m, 2H) 6.75 (dd, J=9.03, 2.20 Hz, 1H) 6.78 (d, J=7.81 Hz, 1H) 6.89 (t, J=7.32 Hz, 1H) 7.01 (t, J=7.57 Hz, 1H) 7.74 (d, J=7.32 Hz, 1H) 9.36 (d, J=8.79 Hz, 1H) 10.20 (s, 1H); LR MS (EI): 377 (M+).

### Example 50

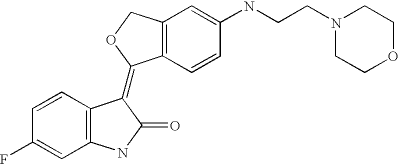

### 6-Fluoro-3-[5-(2-morpholin-4-yl-ethylamino)-3H-isobenzofuran-1-ylidene]-1,3-dihydro-indol-2-one

¹H NMR (500 MHz, DMSO-d₆) δ ppm 2.44 (brs, 4H) 2.53 (t, J=6.59 Hz, 2H) 3.27 (q, J=6.18 Hz, 2H) 3.59 (t, J=4.39 Hz, 4H) 5.63 (s, 2H) 6.59 (dd, J=9.28, 2.44 Hz, 1H) 6.70 (m, 3H) 6.75 (dd, J=9.03, 2.20 Hz, 1H) 7.70 (dd, J=8.54, 5.61 Hz, 1H) 9.31 (d, J=8.79 Hz, 1H) 10.36 (s, 1H).

### Example 51

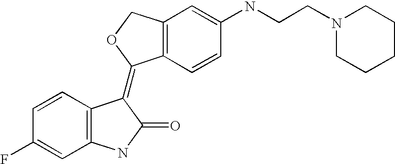

### 6-Fluoro-3-[5-(2-piperidin-1-yl-ethylamino)-3H-isobenzofuran-1-ylidene]-1,3-dihydro-indol-2-one

¹H NMR (500 MHz, DMSO-d₆) δ ppm 1.38 (m, 2H) 1.51 (m, 4H) 2.40 (brs, 4H) 2.48 (m, 2H) 3.24 (q, J=6.35 Hz, 2H) 5.62 (s, 2H) 6.59 (dd, J=9.52, 2.69 Hz, 1H) 6.70 (m, 3H) 6.74 (dd, J=8.79, 1.95 Hz, 1H) 7.70 (dd, J=8.54, 5.61 Hz, 1H) 9.30 (d, J=8.79 Hz, 1H) 10.35 (s, 1H).

### Example 52

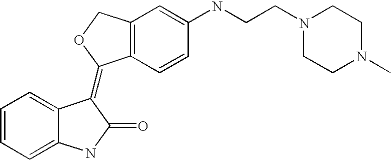

### 3-{5-[2-(4-Methyl-piperazin-1-yl)-ethylamino]-3H-isobenzofuran-1-ylidene}-1,3-dihydro-indol-2-one

¹H NMR (500 MHz, DMSO-d₆) δ ppm 2.16 (s, 3H) 2.34 (brs, 4H) 2.45 (brs, 4H) 2.53 (m, 2H) 3.25 (m, 2H) 5.63 (s, 2H) 6.66 (t, J=5.37 Hz, 1H) 6.71 (s, 1H) 6.76 (m, 2H) 6.89 (t, J=7.57 Hz, 1H) 7.01 (t, J=7.57 Hz, 1H) 7.74 (d, J=7.81 Hz, 1H) 9.36 (d, J=8.79 Hz, 1H) 10.20 (s, 1H).

### Example 53

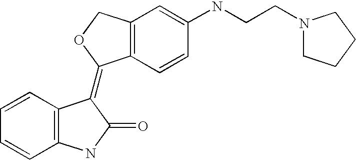

### 3-[5-(2-Pyrrolidin-1-yl-ethylamino)-3H-isobenzofuran-1-ylidene]-1,3-dihydro-indol-2-one

¹H NMR (500 MHz, DMSO-d₆) δ ppm 1.70 (brs, 4H) 2.50 (brs, 4H) 2.64 (t, J=6.59 Hz, 2H) 3.26 (q, J=6.35 Hz, 2H) 5.63 (s, 2H) 6.75 (m, 4H) 6.89 (t, J=7.81 Hz, 1H) 7.01 (t, J=7.08 Hz, 1H) 7.74 (d, J=7.32 Hz, 1H) 9.36 (d, J=8.79 Hz, 1H) 10.20 (s, 1H).

### Example 54

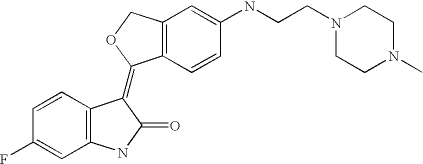

### 6-Fluoro-3-{5-[2-(4-methyl-piperazin-1-yl)-ethylamino]-3H-isobenzofuran-1-ylidene}-1,3-dihydro-indol-2-one

¹H NMR (500 MHz, DMSO-d₆) δ ppm 2.16 (s, 3H) 2.36 (br, 4H) 2.45 (br, 4H) 2.53 (m, 2H) 3.25 (q, J=6.35 Hz, 2H) 5.62 (s, 2H) 6.59 (dd, J=9.28, 2.44 Hz, 1H) 6.71 (m, 4H) 7.70 (dd, J=8.79, 5.86 Hz, 1H) 9.31 (m, J=8.79 Hz, 1H) 10.36 (s, 1H); LR MS (FAB+): 409 (M+1).

### Example 55

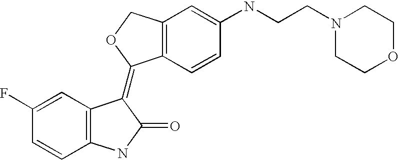

### 5-Fluoro-3-[5-(2-morpholin-4-yl-ethylamino)-3H-isobenzofuran-1-ylidene]-1,3-dihydro-indol-2-one

¹H NMR (500 MHz, DMSO-d₆) δ ppm 2.43 (brs, 4H) 2.53 (t, J=6.59 Hz, 2H) 3.27 (q, J=6.35 Hz, 2H) 3.59 (t, J=4.39 Hz, 4H) 5.65 (s, 2H) 6.78 (m, 5H) 7.48 (dd, J=9.76, 2.44 Hz, 1H) 9.35 (d, J=9.28 Hz, 1H) 10.22 (s, 1H).

### Example 56

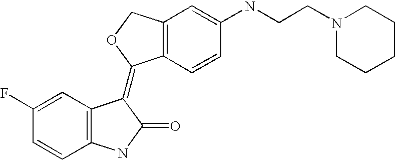

### 5-Fluoro-3-[5-(2-piperidin-1-yl-ethylamino)-3H-isobenzofuran-1-ylidene]-1,3-dihydro-indol-2-one

¹H NMR (500 MHz, DMSO-d₆) δ ppm 1.39 (m, 2H) 1.51 (m, 4H) 2.41 (brs, 4H) 2.49 (m, 2H) 3.26 (m, 2H) 5.65 (s, 2H) 6.74 (m, 4H) 6.81 (m, 1H) 7.48 (dd, J=10.01, 2.68 Hz, 1H) 9.34 (d, J=9.27 Hz, 1H) 10.22 (s, 1H).

### Example 57

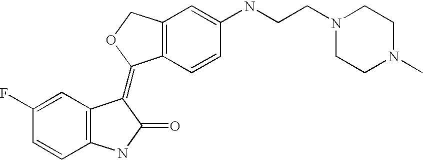

### 5-Fluoro-3-{5-[2-(4-methyl-piperazin-1-yl)-ethylamino]-3H-isobenzofuran-1-ylidene}-1,3-dihydro-indol-2-one

¹H NMR (500 MHz, DMSO-d₆) δ ppm 2.16 (s, 3H) 2.35 (brs, 4H) 2.44 (brs, 4H) 2.53 (m, 2H) 3.26 (q, J=6.02 Hz, 2H) 5.65 (s, 2H) 6.77 (m, 5H) 7.48 (dd, J=10.01, 2.20 Hz, 1H) 9.34 (d, J=9.28 Hz, 1H) 10.22 (s, 1H).

### Example 58

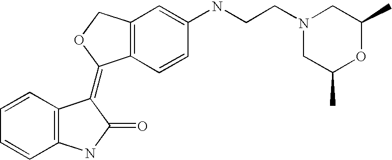

### 3-{5-[2-((2R,6S)-2,6-Dimethyl-morpholin-4-yl)-ethylamino]-3H-isobenzofuran-1-ylidene}-1,3-dihydro-indol-2-one

¹H NMR (500 MHz, DMSO-d₆) δ ppm 1.05 (d, J=6.35 Hz, 6H) 1.67 (t, J=10.74 Hz, 2H) 2.50 (t, J=5.86 Hz, 2H) 2.80 (d, J=10.74 Hz, 2H) 3.26 (q, J=5.86 Hz, 2H) 3.57 (m, 2H) 5.62 (s, 2H) 6.67 (t, J=5.13 Hz, 1H) 6.70 (s, 1H) 6.74 (d, J=8.79 Hz, 1H) 6.77 (d, J=7.32 Hz, 1H) 6.89 (t, J=7.32 Hz, 1H) 7.00 (t, J=7.08 Hz, 1H) 7.74 (d, J=7.32 Hz, 1H) 9.36 (d, J=8.79 Hz, 1H) 10.20 (s, 1H).

### Example 59

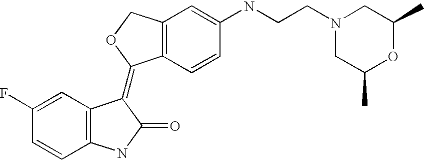

### 3-{5-[2-((2R,6S)-2,6-Dimethyl-morpholin-4-yl)-ethylamino]-3H-isobenzofuran-1-ylidene}-5-fluoro-1,3-dihydro-indol-2-one

¹H NMR (500 MHz, DMSO-d₆) δ ppm 1.05 (d, J=6.35 Hz, 6H) 1.67 (t, J=10.50 Hz, 2H) 2.50 (t, J=6.35 Hz, 2H) 2.80 (d, J=10.74 Hz, 2H) 3.27 (q, J=6.35 Hz, 2H) 3.57 (m, 2H) 5.65 (s, 2H) 6.77 (m, 5H) 7.48 (dd, J=9.76, 2.93 Hz, 1H) 9.34 (d, J=8.79 Hz, 1H) 10.22 (s, 1H).

### Example 60

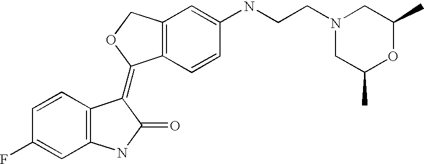

### 3-{5-[2-((2R,6S)-2,6-Dimethyl-morpholin-4-yl)-ethylamino]-3H-isobenzofuran-1-ylidene}-6-fluoro-1,3-dihydro-indol-2-one

¹H NMR (500 MHz, DMSO-d₆) δ ppm 1.05 (d, J=6.35 Hz, 6H) 1.67 (t, J=10.74 Hz, 2H) 2.50 (t, J=6.35 Hz, 2H) 2.80 (d, J=10.74 Hz, 2H) 3.26 (q, J=6.35 Hz, 2H) 3.57 (m, 2H) 5.62 (s, 2H) 6.58 (dd, J=9.28, 2.93 Hz, 1H) 6.70 (m, 3H) 6.74 (dd, J=9.03, 2.20 Hz, 1H) 7.70 (dd, J=8.30, 5.86 Hz, 1H) 9.31 (d, J=8.79 Hz, 1H) 10.35 (s, 1H).

### Example 61

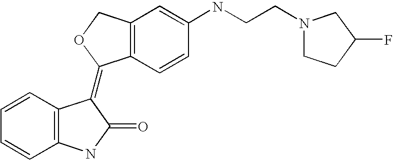

### 3-{5-[2-(3-Fluoro-pyrrolidin-1-yl)-ethylamino]-3H-isobenzofuran-1-ylidene}-1,3-dihydro-indol-2-one

¹H NMR (500 MHz, DMSO-d₆) δ ppm 1.9-2.2 (m, 4H) 2.65 (brs, 2H) 2.86 (brs, 2H) 3.29 (m, 2H) 5.21 (two broad peaks, J_{H-F}=56.14 Hz, 1H) 5.63 (s, 2H) 6.75 (m, 4H) 6.89 (td, J=7.57, 0.98 Hz, 1H) 7.00 (td, J=7.57, 0.98 Hz, 1H) 7.74 (d, J=7.81 Hz, 1H) 9.36 (d, J=8.79 Hz, 1H) 10.20 (s, 1H)

### Example 62

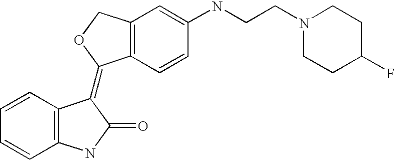

### 3-{5-[2-(4-Fluoro-piperidin-1-yl)-ethylamino]-3H-isobenzofuran-1-ylidene}-1,3-dihydro-indol-2-one

¹H NMR (500 MHz, DMSO-d₆) δ ppm 1.71 (m, 2H) 1.86 (m, 2H) 2.37 (brs, 2H) 2.53 (t, J=6.59 Hz, 2H) 2.60 (brs, 2H) 3.25 (q, J=6.35 Hz, 2H) 4.68 (two broad peaks, J_{H-F}=49.3 Hz, 1H) 5.62 (s, 2H) 6.72 (m, 4H) 6.89 (t, J=7.08 Hz, 1H) 7.00 (t, J=7.57 Hz, 1H) 7.74 (d, J=7.32 Hz, 1H) 9.36 (d, J=8.79 Hz, 1H) 10.20 (s, 1H).

### Example 63

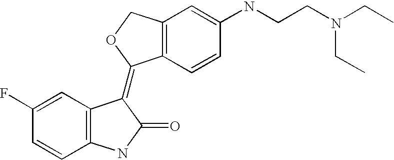

### 3-[5-(2-Diethylamino-ethylamino)-3H-isobenzofuran-1-ylidene]-5-fluoro-1,3-dihydro-indol-2-one

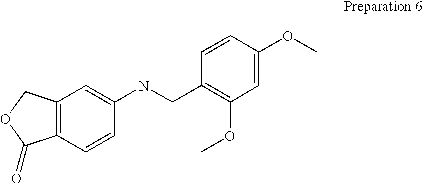

### Preparation of 5-(2,4-Dimethoxy-benzylamino)-3H-isobenzofuran-1-one

To a stirred solution of 5-aminophthalide (11.7 g, 78 mmol) and 2,4-dimethoxybenzaldehyde (15.5 g, 93.6 mmol) in 1% AcOH/DMF (60 ml), was added sodium triacetoxyborohydride (50.0 g, 236 mmol). The mixture was stirred at room temperature for 16 hours, diluted with EtOAc (400 ml), washed with saturated NaHCO₃ solution (3×400 ml) and water (400 ml), dried over anhydrous Na₂SO₄, and evaporated to provide a crude product. Trituration of the crude product with MeOH gave 5-(2,4-dimethoxy-benzylamino)-3H-isobenzofuran-1-one as an off-white powder (19.0 g, 82%).

¹H NMR (500 MHz, DMSO-d₆) δ ppm 3.74 (s, 3H) 3.82 (s, 3H) 4.21 (d, J=5.86 Hz, 2H) 5.15 (s, 2H) 6.48 (dd, J=8.54, 2.20 Hz, 1H) 6.58 (m, 2H) 6.73 (d, J=6.83 Hz, 1H) 7.12 (d, J=8.30 Hz, 2H) 7.46 (d, J=8.79 Hz, 1H).

### Example 64

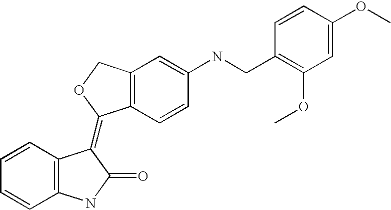

### 3-[5-(2,4-Dimethoxy-benzylamino)-3H-isobenzofuran-1-ylidene]-1,3-dihydro-indol-2-one

To a stirred solution of oxindole (555 mg, 4.17 mmol) in anhydrous dimethoxyethane (20 ml) under nitrogen was added 2.5M n-BuLi solution in hexane (3.67 ml, 9.17 mmol), and the resulting mixture was stirred at room temperature for 10 minutes, followed by the addition of 5-(2,4-dimethoxy-benzylamino)-3H-isobenzofuran-1-one (1.0 g, 3.34 mmol). After stirred at room temperature for 2.5 hours the mixture was poured into 1M HCl aqueous solution (100 ml). The resulting solid was separated, washed with water, and dried under vacuum to afford a crude product. Trituration of the crude product with MeOH produced 3-[5-(2,4-dimethoxy-benzylamino)-3H-isobenzofuran-1-ylidene]-1,3-dihydro-indol-2-one as a yellow solid (630 mg, 46%).

¹H NMR (500 MHz, DMSO-d₆) δ ppm 3.74 (s, 3H) 3.83 (s, 3H) 4.25 (d, J=5.37 Hz, 2H) 5.60 (s, 2H) 6.49 (dd, J=8.30, 2.44 Hz, 1H) 6.59 (d, J=1.95 Hz, 1H) 6.67 (s, 1H) 6.76 (m, 2H) 6.89 (m, 1H) 7.00 (m, 1H) 7.14 (m, 2H) 7.73 (d, J=7.81 Hz, 1H) 9.35 (d, J=8.79 Hz, 1H) 10.19 (s, 1H).

### Example 65

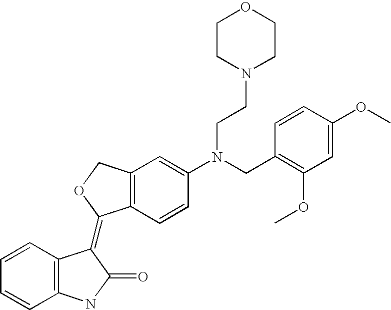

### 3-{5-[(2,4-Dimethoxy-benzyl)-(2-morpholin-4-yl-ethyl)-amino]-3H-isobenzofuran-1-ylidene}-1,3-dihydro-indol-2-one

A mixture of 3-[5-(2,4-dimethoxy-benzylamino)-3H-isobenzofuran-1-ylidene]-1,3-dihydro-indol-2-one (100 mg, 0.24 mmol), 4-(2-iodo-ethyl)-morpholine (90 mg, 0.37 mmol), N,N-diisopropylethylamine (63 mg, 0.49 mmol) and silver triflate (75 mg (0.29 mmol) in 1,4-dioxane (5 ml) was heated at 85° C. under nitrogen for 16 hours. Purification of the mixture by silica gel chromatography, eluted with a gradient of MeOH in CHCl₃ led to 3-{5-[(2,4-dimethoxy-benzyl)-(2-morpholin-4-yl-ethyl)-amino]-3H-isobenzofuran-1-ylidene}-1,3-dihydro-indol-2-one as a yellow solid (40 mg, 31%).

¹H NMR (500 MHz, CDCl₃) δ ppm 2.51 (brs, 4H) 2.65 (t, J=6.83 Hz, 2H) 3.64 (t, J=6.59 Hz, 2H) 3.71 (brs, 4H) 3.79 (s, 3H) 3.86 (s, 3H) 4.57 (s, 2H) 5.52 (s, 2H) 6.38 (dd, J=8.30, 2.44 Hz, 1H) 6.50 (d, J=2.44 Hz, 1H) 6.59 (s, 1H) 6.85 (m, 3H) 7.01 (t, J=7.57 Hz, 1H) 7.08 (t, J=7.57 Hz, 1H) 7.49 (s, 1H) 7.91 (d, J=7.32 Hz, 1H) 9.50 (d, J=8.79 Hz, 1H).

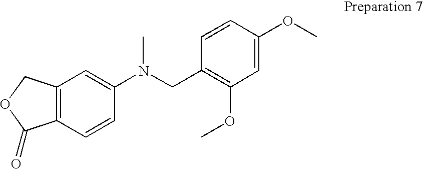

### Preparation of 5-[(2,4-Dimethoxy-benzyl)-methyl-amino]-3H-isobenzofuran-1-one

To a stirred suspension of 5-(2,4-dimethoxy-benzylamino)-3H-isobenzofuran-1-one (18.0 g, 60 mmol) in acetonitrile (200 ml), was added 37% formaldehyde aqueous solution (44.7 ml, 600 mmol) and sodium cyanoborohydride (8.31 g, 132 mmol). The mixture was cooled to 0° C., followed by addition of 10% AcOH aqueous solution (150 ml). The mixture was stirred from 0° C. to room temperature during 2.5 hour period. The resulting solid was filtered, washed with acetonitrile and dried under vacuum to give 5-[(2,4-dimethoxy-benzyl)-methyl-amino]-3H-isobenzofuran-1-one as off-white powder (15.3 g, 81%).

¹H NMR (500 MHz, DMSO-d₆) δ ppm 3.11 (s, 3H) 3.72 (s, 3H) 3.82 (s, 3H) 4.54 (s, 2H) 5.20 (s, 2H) 6.44 (dd, J=8.54, 2.20 Hz, 1H) 6.60 (d, J=2.44 Hz, 1H) 6.75 (s, 1H) 6.80 (d, 2H) 7.55 (d, J=8.79 Hz, 1H); LR MS (EI): 313 (M⁺).

### Example 66

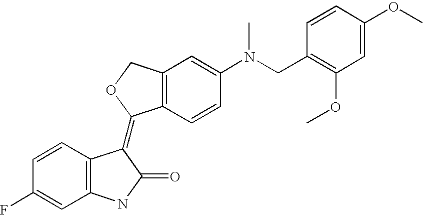

### 3-{5-[(2,4-Dimethoxy-benzyl)-methyl-amino]-3H-isobenzofuran-1-ylidene}-6-fluoro-1,3-dihydro-indol-2-one

To a stirred solution of 6-fluorooxindole (0.60 g, 3.99 mmol) in anhydrous dimethoxyethane (20 ml) under nitrogen was added 2.5M n-BuLi solution in hexane (3.5 ml, 8.75 mmol). After the reaction was stirred at room temperature for 10 minutes, 5-[(2,4-dimethoxy-benzyl)-methyl-amino]-3H-isobenzofuran-1-one (1.0 g, 3.19 mmol) was added. After stirred at room temperature for 2.5 hours, the reaction mixture was poured into 1M HCl aqueous solution (70 ml), and then basified with NaOH aqueous solution. The solid was separated, washed with water, and dried under vacuum to give a crude product. Trituration of the crude product with benzene yielded 3-{5-[(2,4-dimethoxy-benzyl)-methyl-amino]-3H-isobenzofuran-1-ylidene}-6-fluoro-1,3-dihydro-indol-2-one as a yellow solid (0.91 g, 64%).

¹H NMR (500 MHz, DMSO-d₆) δ ppm 3.11 (s, 3H) 3.71 (s, 3H) 3.81 (s, 3H) 4.55 (s, 2H) 5.62 (s, 2H) 6.43 (dd, J=8.30, 2.44 Hz, 1H) 6.58 (m, 2H) 6.68 (m, 1H) 6.83 (m, 3H) 7.68 (dd, J=8.54, 5.61 Hz, 1H) 9.33 (d, J=9.28 Hz, 1H) 10.34 (s, 1H); LR MS (EI): 446 (M+).

The following Example 67 through 76 were prepared using the experiment procedure described in Example 66, but with the appropriate reagent, reaction conditions and reactant substitutions that will be readily realized by those of ordinary skill in this art without the exercise of undue experimentation.

### Example 67

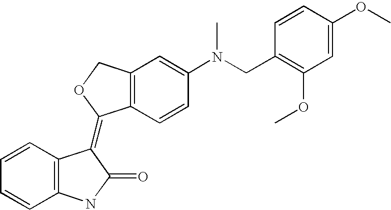

### 3-{5-[(2,4-Dimethoxy-benzyl)-methyl-amino]-3H-isobenzofuran-1-ylidene}-1,3-dihydro-indol-2-one

¹H NMR (500 MHz, DMSO-d₆) δ ppm 3.13 (s, 3H) 3.73 (s, 3H) 3.83 (s, 3H) 4.57 (s, 2H) 5.63 (s, 2H) 6.45 (dd, J=8.30, 2.44 Hz, 1H) 6.61 (d, J=2.44 Hz, 1H) 6.84 (m, 5H) 7.01 (m, 1H) 7.74 (d, J=7.32 Hz, 1H) 9.40 (d, J=8.79 Hz, 1H) 10.21 (s, 1H); LR MS (EI): 428 (M⁺).

### Example 68

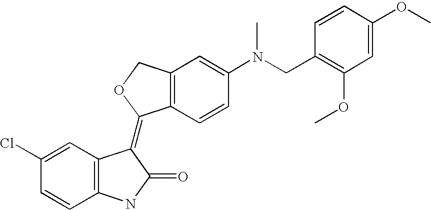

### 5-Chloro-3-{5-[(2,4-dimethoxy-benzyl)-methyl-amino]-3H-isobenzofuran-1-ylidene}-1,3-dihydro-indol-2-one

¹H NMR (500 MHz, DMSO-d₆) δ ppm 3.12 (s, 3H) 3.71 (s, 3H) 3.81 (s, 3H) 4.56 (s, 2H) 5.66 (s, 2H) 6.43 (dd, J=8.30, 2.44 Hz, 1H) 6.59 (d, J=2.44 Hz, 1H) 6.76 (d, J=7.81 Hz, 1H) 6.84 (m, 3H) 7.02 (dd, J=8.30, 1.95 Hz, 1H) 7.69 (d, J=1.95 Hz, 1H) 9.36 (d, J=9.28 Hz, 1H) 10.33 (s, 1H); LR MS (EI): 462 (M+) 464 (M+2).

### Example 69

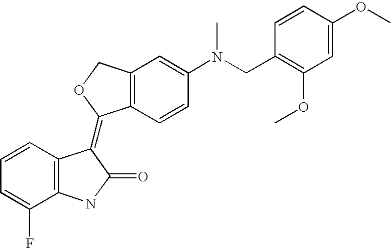

### 3-{5-[(2,4-Dimethoxy-benzyl)-methyl-amino]-3H-isobenzofuran-1-ylidene}-7-fluoro-1,3-dihydro-indol-2-one

¹H NMR (500 MHz, DMSO-d₆) δ ppm 3.12 (s, 3H) 3.71 (s, 3H) 3.81 (s, 3H) 4.56 (s, 2H) 5.64 (s, 2H) 6.43 (dd, J=8.30, 2.44 Hz, 1H) 6.59 (d, J=1.95 Hz, 1H) 6.86 (m, 5H) 7.56 (m, 1H) 9.38 (d, J=9.28 Hz, 1H) 10.65 (s, 1H); LR MS (EI): 446 (M⁺).

### Example 70

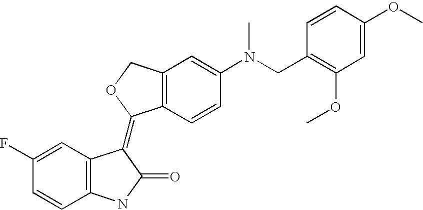

### 3-{5-[(2,4-Dimethoxy-benzyl)-methyl-amino]-3H-isobenzofuran-1-ylidene}-5-fluoro-1,3-dihydro-indol-2-one

¹H NMR (500 MHz, DMSO-d₆) δ ppm 3.12 (s, 3H) 3.71 (s, 3H) 3.81 (s, 3H) 4.56 (s, 2H) 5.64 (s, 2H) 6.43 (dd, J=8.54, 2.20 Hz, 1H) 6.59 (d, J=1.95 Hz, 1H) 6.72 (dd, J=8.54, 4.64 Hz, 1H) 6.82 (m, 4H) 7.46 (dd, J=10.01, 2.69 Hz, 1H) 9.37 (d, J=9.28 Hz, 1H) 10.21 (s, 1H); LR MS (EI): 446 (M⁺).

### Example 71

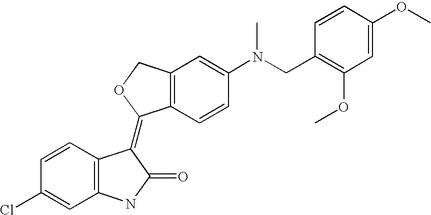

### 6-Chloro-3-{5-[(2,4-dimethoxy-benzyl)-methyl-amino]-3H-isobenzofuran-1-ylidene}-1,3-dihydro-indol-2-one

¹H NMR (500 MHz, DMSO-d₆) δ ppm 3.12 (s, 3H) 3.71 (s, 3H) 3.81 (s, 3H) 4.56 (s, 2H) 5.63 (s, 2H) 6.43 (dd, J=8.30, 1.95 Hz, 1H) 6.59 (d, J=2.44 Hz, 1H) 6.83 (m, 4H) 6.91 (dd, J=8.30, 1.95 Hz, 1H) 7.68 (d, J=8.30 Hz, 1H) 9.34 (d, J=9.28 Hz, 1H) 10.35 (s, 1H); LR MS (EI): 462 (M⁺) 464 (M+2).

### Example 72

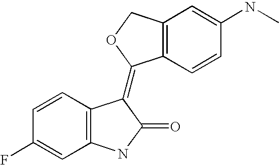

### 6-Fluoro-3-(5-methylamino-3H-isobenzofuran-1-ylidene)-1,3-dihydro-indol-2-one

To a stirred suspension of (3-{5-[(2,4-dimethoxy-benzyl)-methyl-amino]-3H-isobenzofuran-1-ylidene}-6-fluoro-1,3-dihydro-indol-2-one (197 mg, 0.44 mmol) in methylene chloride (10 ml) was added trifluoroacetic acid (1 ml). The mixture was stirred for 30 minutes at room temperature and then evaporated to give a residue. The residue was treated with 50% MeOH in water (25 ml, containing 1 ml of triethylamine) with heating for 30 minutes. After filtered and dried under vacuum, the resulting solid was mixed with acetone (50 ml), and heated at 50° C. for 30 minutes. The mixture was cooled to room temperature and filtered. The filtrate solution was concentrated under reduced pressure to give a crude product. Trituration of the crude product with CHCl₃/hexanes afforded 6-fluoro-3-(5-methylamino-3H-isobenzofuran-1-ylidene)-1,3-dihydro-indol-2-one as a yellow solid (100 mg, 76%).

¹H NMR (500 MHz, DMSO-d₆) δ ppm 2.77 (d, J=4.88 Hz, 3H) 5.62 (s, 2H) 6.57 (dd, J=9.28, 2.44 Hz, 1H) 6.63 (s, 1H) 6.69 (m, 2H) 6.85 (q, J=4.88 Hz, 1H) 7.69 (dd, J=8.30, 5.86 Hz, 1H) 9.31 (d, J=8.79 Hz, 1H) 10.34 (s, 1H); LR MS (EI): 296 (M⁺).

The following Example 73 through 76 were prepared using the experiment procedure described in Example 72, but with the appropriate reagent, reaction conditions and reactant substitutions that will be readily realized by those of ordinary skill in this art without the exercise of undue experimentation

### Example 73

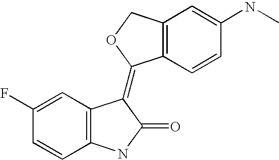

### 5-Fluoro-3-(5-methylamino-3H-isobenzofuran-1-ylidene)-1,3-dihydro-indol-2-one

¹H NMR (500 MHz, DMSO-d₆) δ ppm 2.78 (d, J=4.88 Hz, 3H) 5.64 (s, 2H) 6.64 (s, 1H) 6.68 (dd, J=8.79, 2.44 Hz, 1H) 6.72 (dd, J=8.30, 4.88 Hz, 1H) 6.80 (m, 1H) 6.92 (q, J=4.39 Hz, 1H) 7.46 (dd, J=10.01, 2.69 Hz, 1H) 9.34 (d, J=8.79 Hz, 1H) 10.20 (s, 1H); LR MS (EI): 296 (M⁺).

### Example 74

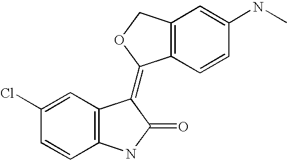

### 5-Chloro-3-(S-methylamino-3H-isobenzofuran-1-ylidene)-1,3-dihydro-indol-2-one

¹H NMR (500 MHz, DMSO-d₆) δ ppm 2.78 (d, J=4.39 Hz, 3H) 5.66 (s, 2H) 6.65 (s, 1H) 6.69 (m, 1H) 6.76 (d, J=8.30 Hz, 1H) 6.94 (m, 1H) 7.01 (dd, J=8.30, 1.95 Hz, 1H) 7.69 (d, J=2.44 Hz, 1H) 9.33 (d, J=8.79 Hz, 1H) 10.32 (s, 1H); LR MS (EI): 312 (M⁺) 314 (M+2).

### Example 75

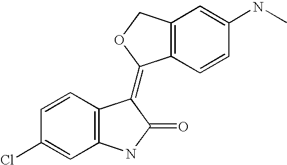

### 6-Chloro-3-(5-methylamino-3H-isobenzofuran-1-ylidene)-1,3-dihydro-indol-2-one

¹H NMR (500 MHz, DMSO-d₆) δ ppm 2.78 (d, J=4.88 Hz, 3H) 5.63 (s, 2H) 6.64 (s, 1H) 6.68 (dd, J=9.03, 2.20 Hz, 1H) 6.76 (d, J=1.95 Hz, 1H) 6.92 (m, 2H) 7.69 (d, J=8.30 Hz, 1H) 9.32 (d, J=8.79 Hz, 1H) 10.34 (s, 1H); LR MS (EI): 312 (M⁺) 314 (M+2).

### Example 76

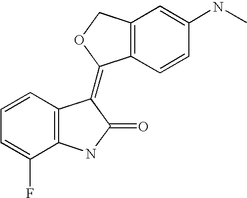

### 7-Fluoro-3-(5-methylamino-3H-isobenzofuran-1-ylidene)-1,3-dihydro-indol-2-one

¹H NMR (500 MHz, DMSO-d₆) δ ppm 2.78 (d, J=4.88 Hz, 3H) 5.64 (s, 2H) 6.65 (s, 1H) 6.69 (d, J=8.79 Hz, 1H) 6.88 (m, 2H) 6.93 (q, J=4.72 Hz, 1H) 7.57 (m, 1H) 9.36 (d, J=8.79 Hz, 1H) 10.64 (s, 1H); LR MS (EI): 296 (M⁺).

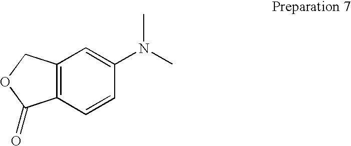

### Preparation of 5-Dimethylamino-3H-isobenzofuran-1-one

To a stirred suspension of 5-aminophthalide (5.00 g, 33.5 mmol) in acetonitrile (120 ml), was added 37% formaldehyde aqueous solution (24.9 ml, 335 mmol) and sodium cyanoborohydride (8.42 g, 134 mmol). The mixture was cooled to 0° C., followed by addition of 10% AcOH aqueous solution (120 ml). The mixture was warmed to room temperature from 0° C. during 1.5 hour-period. The mixture was concentrated under reduced pressure to a smaller volume and was extracted with EtOAc (2×125 ml). The combined organic layers were washed with saturated NaHCO₃ solution (125 ml) and brine (125 ml), dried over Na₂SO₄. Removal of the solvent produced a crude product. Recrystallization of the crude product from MeOH gave 5-dimethylamino-3H-isobenzofuran-1-one as an off-white solid (3.90 g, 66%).

¹H NMR (500 MHz, DMSO-D₆) δ ppm 3.04 (s, 6H) 5.23 (s, 2H) 6.77 (s, 1H) 6.85 (dd, J=8.79, 2.44 Hz, 1H) 7.58 (d, J=8.79 Hz, 1H).

### Example 77

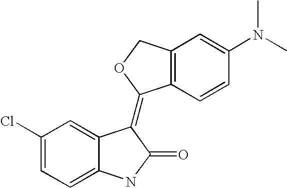

### 5-Chloro-3-(5-dimethylamino-3H-isobenzofuran-1-ylidene)-1,3-dihydro-indol-2-one

To a stirred solution of 5-chlorooxindole (1.18 g, 7.05 mmol) in anhydrous dimethoxyethane (20 ml) under nitrogen was added 2.5M n-BuLi solution in hexane (6.2 ml, 15.5 mmol). The mixture was stirred at room temperature for 10 minutes, and then 5-dimethylamino-3H-isobenzofuran-1-one (1.00 g, 5.64 mmol) was added. After stirred at room temperature for 3 hours, the mixture was poured into 0.5M HCl aqueous solution (80 ml) with stirring and then basified with NaOH aqueous solution. The solid was filtered, washed with water, and dried under vacuum to give a crude product. Trituration of the crude product with MeOH and EtOAc provided 5-chloro-3-(5-dimethylamino-3H-isobenzofuran-1-ylidene)-1,3-dihydro-indol-2-one as a yellow solid (0.90 g, 49%).

¹H NMR (500 MHz, DMSO-d₆) δ ppm 3.06 (s, 6H) 5.69 (s, 2H) 6.77 (d, J=8.30 Hz, 1H) 6.86 (m, 2H) 7.02 (dd, J=8.05, 2.20 Hz, 1H) 7.70 (d, J=1.95 Hz, 1H) 9.40 (m, J=9.28 Hz, 1H) 10.34 (s, 1H); LR MS (EI): 325 (M⁺).

### Example 78

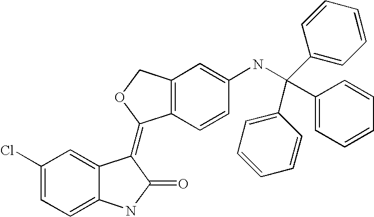

### 5-Chloro-3-[5-(trityl-amino)-3H-isobenzofuran-1-ylidene]-1,3-dihydro-indol-2-one

A solution of 3-(5-Amino-3H-isobenzofuran-1-ylidene)-5-chloro-1,3-dihydro-indol-2-one (75 mg, 0.251 mmol), trityl chloride (84 mg, 0.301 mmol) and triethylamine (53 μl, 0.377 mmol) in DMF (2.0 ml) was stirred at room temperature for 1.25 hours. The mixture was partitioned between EtOAc and water. The EtOAc layer was washed with brine, dried with Na₂SO₄ and rotary evaporated. The yellow solid was precipitated from CHCl₃ to give 5-chloro-3-[5-(trityl-amino)-3H-isobenzofuran-1-ylidene]-1,3-dihydro-indol-2-one as a bright yellow solid (61 mg, 45%).

¹H NMR (500 MHz, DMSO-D6) δ ppm 5.51 (s, 2H) 6.57 (br s, 1H) 6.72 (br s, 1H) 6.75 (d, J=7.81 Hz, 1H) 7.03 (dd, J=8.05, 2.20 Hz, 1H) 7.25 (m, 3H) 7.34 (m, 12H) 7.65 (d, J=1.95 Hz, 1H) 7.94 (s, 1H) 9.07 (d, J=8.79 Hz, 1H) 10.35 (s, 1H).

### Example 17

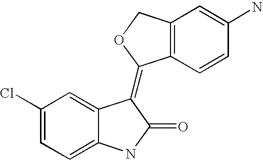

### 3-(5-Amino-3H-isobenzofuran-1-ylidene)-5-chloro-1,3-dihydro-indol-2-one

To a solution of 5-chloro-3-[5-(trityl-amino)-3H-isobenzofuran-1-ylidene]-1,3-dihydro-indol-2-one (200 mg, 0.37 mmol) in THF (4.0 ml) at room temperature was added 1.55M HCl/MeOH (0.5 ml). After stirring for 10 minutes, MeOH (3 ml) was added to the thick mixture, and then the resulting mixture was stirred for another 30 minutes. The precipitant was filtered, and rinsed with MeOH, CHCl₃, MeOH and then 30% EtOAc in hexane to give 3-(5-amino-3H-isobenzofuran-1-ylidene)-5-chloro-1,3-dihydro-indol-2-one as a bright yellow solid (79 mg, 72%).

¹H NMR (500 MHz, DMSO-D6) δ ppm 5.64 (s, 2H) 6.39 (s, 2H) 6.68 (m, 2H) 6.77 (d, J=8.30 Hz, 1H) 7.03 (dd, J=8.05, 2.20 Hz, 1H) 7.70 (d, J=1.95 Hz, 1H) 9.31 (m, 1H) 10.34 (s, 1H).

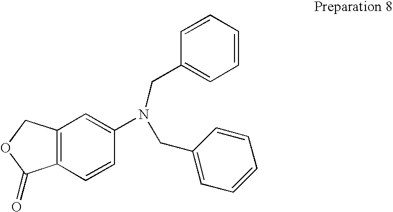

### Preparation of 5-Dibenzylamino-3H-isobenzofuran-1-one

A mixture of 5-amino-2-benzofuran-1(3H)-one (100 mg, 0.67 mmol), benzyl bromide (319 μl, 2.68 mmol), and diisopropylethylamine (350 μl, 2.01 mmol) in THF (2.0 ml) was heated at 50° C. for 65 hours. The reaction was partitioned between water and EtOAc. The organic layer was washed with water, brine, dried with Na₂SO₄ and rotary evaporated. The yellow oil was triturated at room temperature with 10% EtOAc/hexane and then chromatographed (20% to 40% EtOAc/hexane gradient) to give 5-dibenzylamino-3H-isobenzofuran-1-one as a white solid (136 mg, 31%).

¹H NMR (500 MHz, CDCl₃) δ ppm 4.76 (s, 4H) 5.12 (s, 2H) 6.62 (d, J=1.46 Hz, 1H) 6.84 (dd, J=8.79, 1.95 Hz, 1H) 7.22 (m, 4H) 7.30 (m, 2H) 7.37 (m, 4H) 7.67 (d, J=8.79 Hz, 1H).

### Example 79

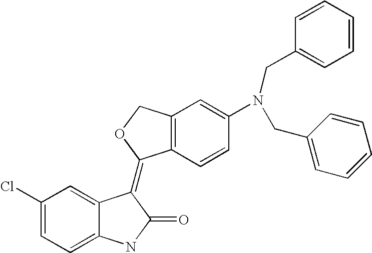

### 5-Chloro-3-(5-dibenzylamino-3H-isobenzofuran-1-ylidene)-1,3-dihydro-indol-2-one

To a solution of 5-chlorooxindole (63 mg, 0.371 mmol) in dimethoxyethane (2.0 ml) at room temperature was added 1.0M LiHMDS in THF (742 μl). After the mixture was stirred for 10 minutes at room temperature, 5-dibenzylamino-3H-isobenzofuran-1-one (110 mg, 0.334 mmol) was added, and the reaction was rapidly stirred at room temperature for 1.5 hours. The mixture was quenched into 4% HCl (20 ml), and then stirred with EtOAc. The mixture was basified with saturated NaHCO₃ aqueous solution. The organic layer was diluted with MeOH, followed by addition of HCl in MeOH to give a yellow precipitant. The yellow precipitant was separated, and rinsed with MeOH and 20% EtOAc/hexane to yield 5-chloro-3-(5-dibenzylamino-3H-isobenzofuran-1-ylidene)-1,3-dihydro-indol-2-one as a yellow solid (36 mg, 23%).

¹H NMR (500 MHz, DMSO-D6) δ ppm 4.87 (s, 4H) 5.64 (s, 2H) 6.77 (d, J=8.30 Hz, 1H) 6.90 (m, 2H) 7.04 (dd, J=8.06, 2.20 Hz, 1H) 7.28 (m, 6H) 7.36 (t, J=7.57 Hz, 4H) 7.69 (d, J=1.95 Hz, 1H) 9.34 (d, J=9.28 Hz, 1H) 10.35 (s, 1H).

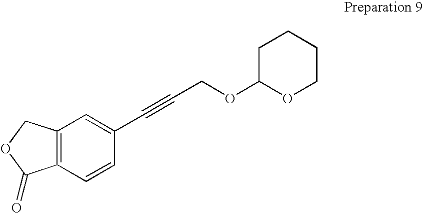

### Preparation of 5-[3-(Tetrahydro-pyran-2-yloxy)-prop-1-ynyl]-3H-isobenzofuran-1-one

To a mixture of 5-bromophthalide (2.00 g, 9.39 mmol), tetrahydro-2-(2-propynyloxy)-2H-pyran (5.3 ml, 37.6 mmol), copper(I) iodide (0.645 g, 3.39 mmol), triethylamine (1.90 g, 18.8 mmol) in DMF (20 ml), was added tetrakis(triphenylphosphine)palladium (1.30 g, 1.13 mmol). The mixture was heated at 65° C. under argon for 16 hours, cooled to room temperature and diluted with diethyl ether (250 ml). The filtrate was washed with brine (4×100 ml), dried over anhydrous Na₂SO₄, and concentrated to give a dark-reddish residue. Purification of the residue mixture by silica gel column chromatography, eluted with a gradient of EtOAc, led to 5-[3-(tetrahydro-pyran-2-yloxy)-prop-1-ynyl]-3H-isobenzofuran-1-one as a white solid (2.23 g, 87%).

¹H NMR (500 MHz, DMSO-d₆) δ ppm 1.50 (m, 4H) 1.69 (m, 2H) 3.49 (m, 1H) 3.77 (m, 1H) 4.50 (m, 2H) 4.83 (m, 1H) 5.40 (s, 2H) 7.63 (d, J=6.83 Hz, 1H) 7.77 (s, 1H) 7.84 (d, J=7.81 Hz, 1H); LR MS (EI): 272 (M⁺).

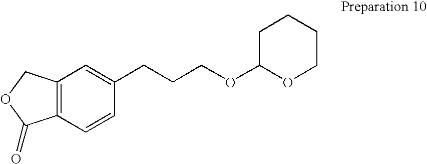

### Preparation of 5-[3-(Tetrahydro-pyran-2-yloxy)-propyl]-3H-isobenzofuran-1-one

A mixture of 5-[3-(tetrahydro-pyran-2-yloxy)-prop-1-ynyl]-3H-isobenzofuran-1-one (1.0 g, 3.67 mmol) and 10% palladium on carbon (200 mg) in MeOH (15 ml) was shaken under 45 psi of hydrogen for 20 hours. The catalyst was removed by filtration through celite and rinsed with MeOH. The combined filtrates were evaporated to give 5-[3-(tetrahydro-pyran-2-yloxy)-propyl]-3H-isobenzofuran-1-one as a light yellow oil (1.0 g, 99%).

¹H NMR (500 MHz, DMSO-d₆) δ ppm 1.45 (m, 4H) 1.61 (m, 1H) 1.71 (m, 1H) 1.88 (m, 2H) 2.79 (m, 2H) 3.38 (m, 2H) 3.64 (m, 1H) 3.72 (m, 1H) 4.53 (t, J=3.42 Hz, 1H) 5.37 (s, 2H) 7.44 (d, J=7.81 Hz, 1H) 7.51 (s, 1H) 7.75 (d, J=7.81 Hz, 1H); LR MS (EI): 276 (M⁺).

### Example 80

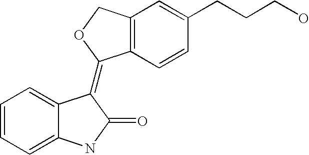

### 3-[5-(3-Hydroxy-propyl)-3H-isobenzofuran-1-ylidene]-1,3-dihydro-indol-2-one

To a stirred solution of oxindole (401 mg, 3.0 mmol) in anhydrous dimethoxyethane (20 ml) under nitrogen was added 1.0M LiHMDS/THF solution (6.3 ml, 6.3 mmol). The mixture was stirred at room temperature for 10 minutes, and 5-[3-(tetrahydro-pyran-2-yloxy)-propyl]-3H-isobenzofuran-1-one (500 mg, 1.81 mmol) was added. After stirred at room temperature for 2.5 hours, the mixture was poured into a mixture of THF (30 ml) and 2M HCl aqueous solution (30 ml) and heated at 65° C. for 1 hour. The mixture was cooled to room temperature, and then poured into an ice water (300 ml). The resulting solid was separated, rinsed with water, and dried to give 3-[5-(3-hydroxy-propyl)-3H-isobenzofuran-1-ylidene]-1,3-dihydro-indol-2-one as a yellow solid (460 mg, 83%).

¹H NMR (500 MHz, DMSO-d₆) δ ppm 1.77 (m, 2H) 2.76 (m, 2H) 3.45 (t, J=6.35 Hz, 2H) 4.53 (brs, 1H) 5.78 (s, 2H) 6.82 (d, J=7.81 Hz, 1H) 6.95 (t, J=7.57 Hz, 1H) 7.10 (t, J=7.81 Hz, 1H) 7.41 (d, J=8.30 Hz, 1H) 7.48 (s, 1H) 7.83 (d, J=7.81 Hz, 1H) 9.55 (d, J=8.30 Hz, 1H) 10.39 (s, 1H).

### Example 81

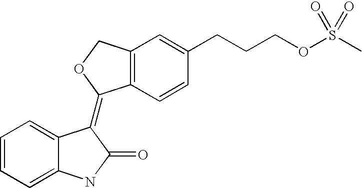

### Methanesulfonic acid 3-[1-(2-oxo-1,2-dihydro-indol-3-ylidene)-1,3-dihydro-isobenzofuran-5-yl]-propyl ester

To a stirred suspension of 3-[5-(3-hydroxy-propyl)-3H-isobenzofuran-1-ylidene]-1,3-dihydro-indol-2-one (440 mg, 1.43 mmol) and triethylamine (289 mg, 2.86 mmol) in THF (7 ml), was added methanesulfonyl chloride (327 mg, 2.86 mmol). The mixture was stirred for 20 minutes and poured into an ice water (150 ml, containing 0.5 ml of AcOH). The solid was filtered, washed with water and dried under vacuum to afford a crude product. The crude product was purified by silica gel column chromatography, eluted with 5% MeOH in CHCl₃, to give methanesulfonic acid 3-[1-(2-oxo-1,2-dihydro-indol-3-ylidene)-1,3-dihydro-isobenzofuran-5-yl]-propyl ester as a yellow solid (437 mg, 79%).

¹H NMR (500 MHz, DMSO-d₆) δ ppm 2.05 (m, 2H) 2.84 (m, 2H) 3.20 (s, 3H) 4.24 (t, J=6.35 Hz, 2H) 5.78 (s, 2H) 6.82 (d, J=7.32 Hz, 1H) 6.96 (t, J=7.57 Hz, 1H) 7.10 (m, 1H) 7.45 (d, J=8.30 Hz, 1H) 7.53 (s, 1H) 7.83 (d, J=7.81 Hz, 1H) 9.57 (d, J=8.30 Hz, 1H) 10.40 (s, 1H).

### Example 82

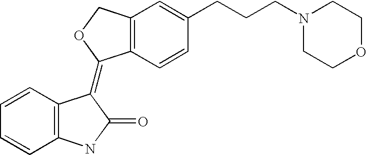

### 3-[5-(3-Morpholin-4-yl-propyl)-3H-isobenzofuran-1-ylidene]-1,3-dihydro-indol-2-one

A mixture of methanesulfonic acid 3-[1-(2-oxo-1,2-dihydro-indol-3-ylidene)-1,3-dihydro-isobenzofuran-5-yl]-propyl ester (400 mg, 1.04 mmol) and morpholine (1.5 ml, 17.2 mmol) in DMF (5 ml) was heated at 90° C. for 1.5 hours. The mixture was cooled, and poured into water (125 ml) with stirring. The solid was filtered, washed with water and dried under vacuum to give 3-[5-(3-morpholin-4-yl-propyl)-3H-isobenzofuran-1-ylidene]-1,3-dihydro-indol-2-one as a yellow solid (385 mg, 98%).

¹H NMR (500 MHz, DMSO-d₆) δ ppm 1.78 (m, 2H) 2.34 (brs, 6H) 2.74 (m, 2H) 3.57 (m, 4H) 5.77 (s, 2H) 6.82 (d, J=7.81 Hz, 1H) 6.95 (m, 1H) 7.10 (t, J=7.57 Hz, 1H) 7.42 (d, J=7.81 Hz, 1H) 7.50 (s, 1H) 7.82 (d, J=7.32 Hz, 1H) 9.54 (d, J=8.30 Hz, 1H) 10.38 (s, 1H).

The following Example 83 was prepared using the experiment procedure described in Example 82, but with the appropriate reagent, reaction conditions and reactant substitutions that will be readily realized by those of ordinary skill in this art without the exercise of undue experimentation.

### Example 83

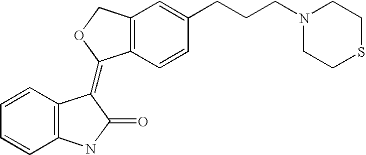

### 3-[5-(3-Thiomorpholin-4-yl-propyl)-3H-isobenzofuran-1-ylidene]-1,3-dihydro-indol-2-one

¹H NMR (500 MHz, DMSO-d₆) δ ppm 1.79 (m, 2H) 2.33 (t, J=7.08 Hz, 2H) 2.60 (brs, 8H) 2.72 (m, 2H) 5.77 (s, 2H) 6.82 (d, J=7.81 Hz, 1H) 6.95 (t, J=7.08 Hz, 1H) 7.10 (t, J=7.08 Hz, 1H) 7.42 (d, J=8.30 Hz, 1H) 7.49 (s, 1H) 7.82 (d, J=7.32 Hz, 1H) 9.54 (d, J=8.30 Hz, 1H) 10.39 (s, 1H).

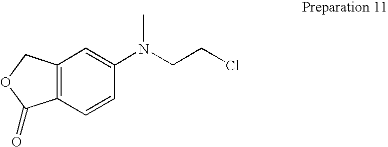

### Preparation of 5-[(2-Chloro-ethyl)-methyl-amino]-3H-isobenzofuran-1-one

A mixture of 5-(2-chloro-ethylamino)-3H-isobenzofuran-1-one (3.0 g, 14.2 mmol), 37% formaldehyde aqueous solution (10 ml, 134 mmol) and sodium cyanoborohydride (3.6 g, 56.8 mmol) in a mixture of 10% AcOH aqueous solution (40 ml) and acetonitrile (40 ml) was stirred at 0° C. for 30 minutes. The mixture was allowed to warm to room temperature and was continuously stirred for 2 hours. The mixture was concentrated under reduced pressure, basified with 1M NaOH aqueous solution, and extracted with EtOAc (3×100 ml). The combined organic layers were washed with 1M NaOH aqueous solution (100 ml) and then water (2×100 ml), dried over anhydrous Na₂SO₄. Removal of the solvent led to a light yellow oil, which was crystallized with diethyl ether to produce 5-[(2-chloro-ethyl)-methyl-amino]-3H-isobenzofuran-1-one as a yellow solid (2.5 g, 78%).

¹H NMR (500 MHz, DMSO-d₆) δ ppm 3.07 (s, 3H) 3.80 (m, 4H) 5.23 (s, 2H) 6.84 (d, J=1.95 Hz, 1H) 6.91 (dd, J=8.79, 2.44 Hz, 1H) 7.59 (d, J=8.30 Hz, 1H).

### Example 84

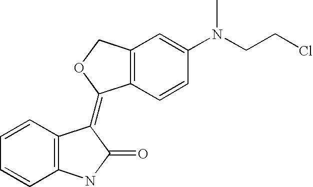

### 3-{5-[(2-Chloro-ethyl)-methyl-amino]-3H-isobenzofuran-1-ylidene}-1,3-dihydro-indol-2-one

To a stirred solution of oxindole (0.739 g, 5.55 mmol) in anhydrous dimethoxyethane (10 ml) under nitrogen was added 1.0M LiHMDS/THF solution (14.0 ml, 14.0 mmol). The mixture was stirred at room temperature for 10 minutes, and 5-[(2-chloro-ethyl)-methyl-amino]-3H-isobenzofuran-1-one (1.0 g, 4.4 mmol) was added. The mixture was stirred at room temperature for 3 hours and was then poured into 1M HCl aqueous solution (300 ml) with stirring. The resulting mixture was heated at 40° C. for 30 minutes. The solid was filtered, washed with water and dried under vacuum to afford 3-{5-[(2-chloro-ethyl)-methyl-amino]-3H-isobenzofuran-1-ylidene}-1,3-dihydro-indol-2-one as a yellow solid (1.20 g, 79%). H NMR (500 MHz, DMSO-d₆) δ ppm 3.10 (s, 3H) 3.83 (m, 4H) 5.67 (s, 2H) 6.79 (d, J=7.32 Hz, 1H) 6.92 (m, 3H) 7.02 (t, J=7.08 Hz, 1H) 7.76 (d, J=7.81 Hz, 1H) 9.45 (d, J=8.79 Hz, 1H) 10.23 (s, 1H).

### Example 85

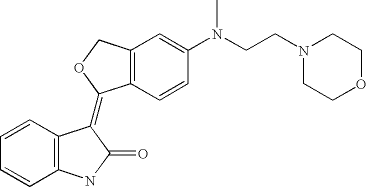

### 3-{5-[Methyl-(2-morpholin-4-yl-ethyl)-amino]-3H-isobenzofuran-1-ylidene}-1,3-dihydro-indol-2-one

A mixture of 3-{5-[(2-chloro-ethyl)-methyl-amino]-3H-isobenzofuran-1-ylidene}-1,3-dihydro-indol-2-one (1.20 g, 3.52 mmol) and morpholine (3 ml, 34.4 mmol) in DMF (5 ml) was heated at 110° C. under nitrogen for 16 hours. The mixture was cooled to room temperature and was then poured into water (100 ml). The solid was filtered, washed with water and dried under vacuum to give a crude product. Purification of the crude product by silica gel column chromatography, eluted with a gradient of MeOH in CHCl₃, resulted in 3-{5-[methyl-(2-morpholin-4-yl-ethyl)-amino]-3H-isobenzofuran-1-ylidene}-1,3-dihydro-indol-2-one as a yellow solid (0.57 g, 41%).

¹H NMR (500 MHz, DMSO-d₆) δ ppm 2.44 (brs, 4H) 2.48 (brs, 2H) 3.06 (s, 3H) 3.56 (t, J=4.39 Hz, 4H) 3.60 (t, J=6.83 Hz, 2H) 5.66 (s, 2H) 6.78 (d, J=7.81 Hz, 1H) 6.83 (s, 1H) 6.88 (m, 2H) 7.01 (m, 1H) 7.75 (d, J=7.32 Hz, 1H) 9.42 (d, J=8.79 Hz, 1H) 10.21 (s, 1H)

The following Example 86 was prepared using the experiment procedure described in Example 85, but with the appropriate reagent, reaction conditions and reactant substitutions that will be readily realized by those of ordinary skill in this art without the exercise of undue experimentation.

### Example 86

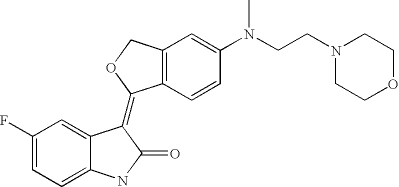

### 5-Fluoro-3-{5-[methyl-(2-morpholin-4-yl-ethyl)-amino]-3H-isobenzofuran-1-ylidene}-1,3-dihydro-indol-2-one

¹H NMR (500 MHz, DMSO-d₆) δ ppm 2.45 (brs, 4H) 2.48 (brs, 2H) 3.07 (s, 3H) 3.56 (t, J=3.91 Hz, 4H) 3.61 (t, J=6.83 Hz, 2H) 5.69 (s, 2H) 6.74 (dd, J=8.54, 4.64 Hz, 1H) 6.85 (m, 3H) 7.50 (m, 1H) 9.41 (d, J=8.79 Hz, 1H) 10.24 (s, 1H).

### Example 87

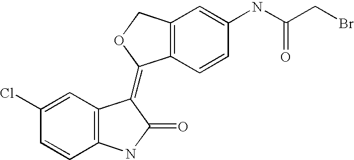

### 2-Bromo-N-[1-(5-chloro-2-oxo-1,2-dihydro-indol-3-ylidene)-1,3-dihydro-isobenzofuran-5-yl]-acetamide

A mixture of 3-(5-amino-3H-isobenzofuran-1-ylidene)-5-chloro-1,3-dihydro-indol-2-one (300 mg, 1.00 mmol), bromoacetic anhydride (350 mg, 1.35 mmol) and potassium carbonate (138 mg, 11.0 mmol) in THF (10 ml) was stirred at 50° C. for 2 hours. The mixture was poured into water (100 ml). The resulting solid was filtered, washed with water, and dried under vacuum to give 2-bromo-N-[1-(5-chloro-2-oxo-1,2-dihydro-indol-3-ylidene)-1,3-dihydro-isobenzofuran-5-yl]-acetamide as a brown solid (380 mg, 91%).

### Example 88

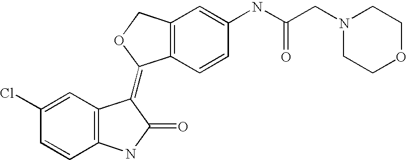

### N-[1-(5-Chloro-2-oxo-1,2-dihydro-indol-3-ylidene)-1,3-dihydro-isobenzofuran-5-yl]-2-morpholin-4-yl-acetamide

A mixture of 2-bromo-N-[1-(5-chloro-2-oxo-1,2-dihydro-indol-3-ylidene)-1,3-dihydro-isobenzofuran-5-yl]-acetamide (81 mg, 0.19 mmol) in morpholine (2 ml) was stirred at 50° C. under nitrogen for 50 minutes, and was then poured into water (75 ml). The solid was filtered, washed with water, and dried under vacuum to give N-[1-(5-chloro-2-oxo-1,2-dihydro-indol-3-ylidene)-1,3-dihydro-isobenzofuran-5-yl]-2-morpholin-4-yl-acetamide as a yellow solid (67 mg, 82%); LR MS (FAB+): 426 (M+1) 428 (M+3).

The following Example 89 through 92 were prepared using the experiment procedure described in Example 88, but with the appropriate reagent, reaction conditions and reactant substitutions that will be readily realized by those of ordinary skill in this art without the exercise of undue experimentation

### Example 89

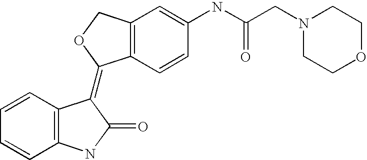

### 2-Morpholin-4-yl-N-[1-(2-oxo-1,2-dihydro-indol-3-ylidene)-1,3-dihydro-isobenzofuran-5-yl]-acetamide

¹H NMR (500 MHz, DMSO-d₆) δ ppm 2.53 (t, J=4.39 Hz, 4H) 3.21 (s, 2H) 3.65 (t, J=4.60 Hz, 4H) 5.78 (s, 2H) 6.82 (d, J=7.32 Hz, 1H) 6.95 (t, J=7.08 Hz, 1H) 7.09 (t, J=7.08 Hz, 1H) 7.65 (dd, J=8.79, 1.95 Hz, 1H) 7.80 (d, J=7.32 Hz, 1H) 8.12 (s, 1H) 9.56 (d, J=8.79 Hz, 1H) 10.22 (s, 1H) 10.38 (s, 1H); LR MS (FAB+): 392 (M+1).

### Example 90

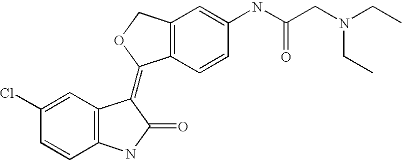

### N-[1-(5-Chloro-2-oxo-1,2-dihydro-indol-3-ylidene)-1,3-dihydro-isobenzofuran-5-yl]-C-diethylamino-acetamide

LR MS (FAB+): 412 (M+1) 414 (M+3).

### Example 91

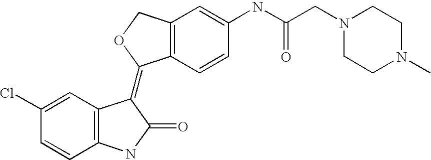

### N-[1-(5-Chloro-2-oxo-1,2-dihydro-indol-3-ylidene)-1,3-dihydro-isobenzofuran-5-yl]-2-(4-methyl-piperazin-1-yl)-acetamide

¹H NMR (500 MHz, DMSO-d₆) δ ppm 2.17 (s, 3H) 2.38 (brs, 4H) 2.53 (brs, 4H) 3.18 (s, 2H) 5.82 (s, 2H) 6.82 (d, J=7.81 Hz, 1H) 7.12 (dd, J=8.06, 2.20 Hz, 1H) 7.66 (d, J=8.30 Hz, 1H) 7.76 (s, 1H) 8.13 (s, 1H) 9.53 (d, J=8.79 Hz, 1H) 10.20 (s, 1H) 10.52 (s, 1H); LR MS (FAB+): 439 (M+1) 441 (M+3).

### Example 92

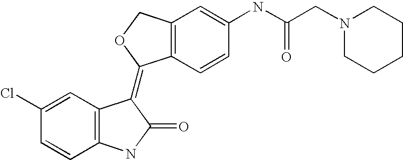

### N-[1-(5-Chloro-2-oxo-1,2-dihydro-indol-3-ylidene)-1,3-dihydro-isobenzofuran-5-yl]-2-piperidin-1-yl-acetamide

¹H NMR (500 MHz, DMSO-d₆) δ ppm 1.41 (br, 2H) 1.57 (m, 4H) 2.47 (br, 4H) 3.14 (s, 2H) 5.82 (s, 2H) 6.82 (d, J=8.30 Hz, 1H) 7.12 (dd, J=8.30, 1.95 Hz, 1H) 7.67 (dd, J=8.79, 1.95 Hz, 1H) 7.76 (d, J=2.44 Hz, 1H) 8.14 (s, 1H) 9.53 (d, J=8.79 Hz, 1H) 10.17 (s, 1H) 10.52 (s, 1H); LR MS (FAB+): 424 (M+1).

### Example 93

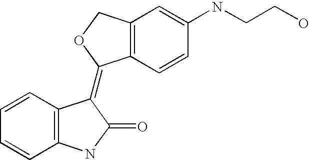

### 3-[5-(2-Hydroxy-ethylamino)-3H-isobenzofuran-1-ylidene]-1,3-dihydro-indol-2-one

¹H NMR (500 MHz, DMSO-d₆) δ ppm 3.23 (q, J=5.86 Hz, 2H) 3.59 (q, J=5.70 Hz, 2H) 4.79 (t, J=5.61 Hz, 1H) 5.63 (s, 2H) 6.77 (m, 4H) 6.89 (t, J=7.57 Hz, 1H) 7.01 (t, J=7.57 Hz, 1H) 7.74 (d, J=7.81 Hz, 1H) 9.36 (d, J=9.28 Hz, 1H) 10.20 (s, 1H).

### Example 94

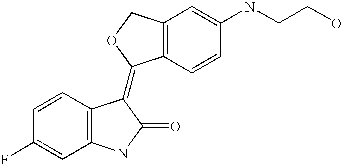

### 6-Fluoro-3-[5-(2-hydroxy-ethylamino)-3H-isobenzofuran-1-ylidene]-1,3-dihydro-indol-2-one

6-Fluoro-3-[5-(2-hydroxy-ethylamino)-3H-isobenzofuran-1-ylidene]-1,3-dihydro-indol-2-one was obtained as the minor product in the preparation of 3-[5-(2-Chloro-ethylamino)-3H-isobenzofuran-1-ylidene]-5-fluoro-1,3-dihydro-indol-2-one (Example 46).

¹H NMR (500 MHz, DMSO-d₆) δ ppm 3.23 (q, J=5.86 Hz, 2H) 3.59 (q, J=5.86 Hz, 2H) 4.79 (t, J=5.37 Hz, 1H) 5.63 (s, 2H) 6.59 (dd, J=9.28, 2.44 Hz, 1H) 6.72 (m, 3H) 6.83 (t, J=5.37 Hz, 1H) 7.70 (dd, J=8.30, 5.86 Hz, 1H) 9.31 (d, J=8.79 Hz, 1H) 10.35 (s, 1H); LR MS (FAB+): 327 (M+1).

### Example 95

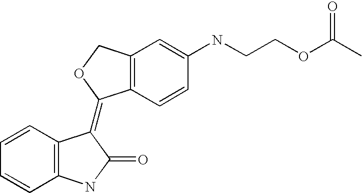

### Acetic acid 2-[1-(2-oxo-1,2-dihydro-indol-3-ylidene)-1,3-dihydro-isobenzofuran-5-ylamino]-ethyl ester

To a stirred mixture of 3-[5-(2-hydroxy-ethylamino)-3H-isobenzofuran-1-ylidene]-1,3-dihydro-indol-2-one (50 mg, 0.16 mmol) and acetic anhydride (20 mg, 0.20 mmol) in THF (3 ml), was added 4-dimethylaminopyridine (10 mg, 0.08 mmol). The mixture was stirred for 30 minutes and was then poured into water (75 ml). The solid was filtered, washed with water and dried under vacuum to give acetic acid 2-[1-(2-oxo-1,2-dihydro-indol-3-ylidene)-1,3-dihydro-isobenzofuran-5-ylamino]-ethyl ester as a yellow solid (39 mg, 68%).

¹H NMR (500 MHz, DMSO-d₆) δ ppm 2.03 (s, 3H) 3.43 (q, J=5.70 Hz, 2H) 4.18 (t, J=5.61 Hz, 2H) 5.64 (s, 2H) 6.77 (m, 3H) 6.90 (t, J=7.08 Hz, 1H) 6.95 (t, J=5.61 Hz, 1H) 7.01 (m, 1H) 7.75 (d, J=7.32 Hz, 1H) 9.38 (d, J=8.79 Hz, 1H) 10.21 (s, 1H).

The following Example 96 was prepared using the experiment procedure described in Example 95, but with the appropriate reagent, reaction conditions and reactant substitutions that will be readily realized by those of ordinary skill in this art without the exercise of undue experimentation.

### Example 96

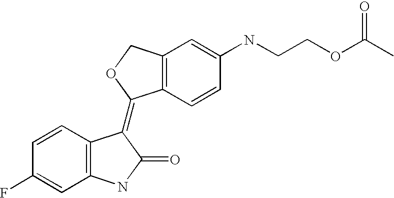

### Acetic acid 2-[1-(6-fluoro-2-oxo-1,2-dihydro-indol-3-ylidene)-1,3-dihydro-isobenzofuran-5-ylamino]-ethyl ester

¹H NMR (500 MHz, DMSO-d₆) δ ppm 2.03 (s, 3H) 3.43 (q, J=5.86 Hz, 2H) 4.17 (t, J=5.61 Hz, 2H) 5.64 (s, 2H) 6.59 (dd, J=9.28, 2.44 Hz, 1H) 6.73 (m, 3H) 6.97 (t, J=5.61 Hz, 1H) 7.71 (dd, J=8.30, 5.86 Hz, 1H) 9.32 (d, J=8.79 Hz, 1H) 10.37 (s, 1H); LR MS (FAB+): 369 (M+1).

### Example 97

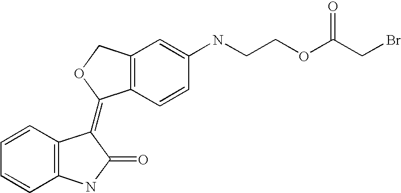

### Bromo-acetic acid 2-[1-(2-oxo-1,2-dihydro-indol-3-ylidene)-1,3-dihydro-isobenzofuran-5-ylamino]-ethyl ester

To a stirred suspension of 3-[5-(2-hydroxy-ethylamino)-3H-isobenzofuran-1-ylidene]-1,3-dihydro-indol-2-one (100 mg, 0.324 mmol) and bromoacetic anhydride (126 mg, 0.486 mmol) in THF (3 ml), was added 4-dimethylaminopyridine (10 mg, 0.082 mmol). The mixture was stirred at room temperature for 1 hour and then poured into water (75 ml). The solid was filtered, washed with water and dried under vacuum to give bromo-acetic acid 2-[1-(2-oxo-1,2-dihydro-indol-3-ylidene)-1,3-dihydro-isobenzofuran-5-ylamino]-ethyl ester as a yellow solid (108 mg, 78%).

¹H NMR (300 MHz, DMSO-d₆) δ ppm 3.46 (m, 2H) 4.17 (s, 2H) 4.29 (t, J=5.42 Hz, 2H) 5.64 (s, 2H) 6.77 (m, 3H) 6.91 (m, 2H) 7.02 (m, 1H) 7.75 (d, J=7.62 Hz, 1H) 9.38 (d, J=9.67 Hz, 1H) 10.21 (s, 1H); LR MS (CI+): 429 (M+1) 431 (M+3).

### Example 98

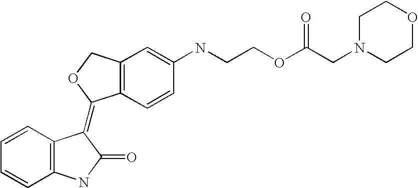

### Morpholin-4-yl-acetic acid 2-[1-(2-oxo-1,2-dihydro-indol-3-ylidene)-1,3-dihydro-isobenzofuran-5-ylamino]-ethyl ester

A mixture of bromo-acetic acid 2-[1-(2-oxo-1,2-dihydro-indol-3-ylidene)-1,3-dihydro-isobenzofuran-5-ylamino]-ethyl ester (35 mg, 0.082 mmol) in morpholine (1 ml) was stirred at room temperature for 1.5 hours. The mixture was poured into 2% AcOH aqueous solution (50 ml) with stirring. The mixture was then basified with NaOH solution. The solid was filtered, washed with water, dried under vacuum to give morpholin-4-yl-acetic acid 2-[1-(2-oxo-1,2-dihydro-indol-3-ylidene)-1,3-dihydro-isobenzofuran-5-ylamino]-ethyl ester as a yellow solid (18 mg, 51%).

¹H NMR (300 MHz, DMSO-d₆) δ ppm 2.47 (m, 4H) 3.23 (s, 2H) 3.45 (m, 2H) 3.55 (m, 4H) 4.23 (t, J=5.42 Hz, 2H) 5.64 (s, 2H) 6.77 (m, 3H) 6.90 (m, 2H) 7.01 (m, 1H) 7.75 (d, J=7.04 Hz, 1H) 9.38 (d, J=9.09 Hz, 1H) 10.21 (s, 1H); LR MS (CI+): 436 (M+1).

The following Example 99 through 101 were prepared using the experiment procedure described in Example 98, but with the appropriate reagent, reaction conditions and reactant substitutions that will be readily realized by those of ordinary skill in this art without the exercise of undue experimentation.

### Example 99

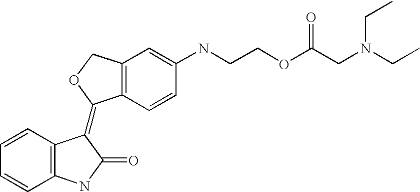

### Diethylamino-acetic acid 2-[1-(2-oxo-1,2-dihydro-indol-3-ylidene)-1,3-dihydro-isobenzofuran-5-ylamino]-ethyl ester

¹H NMR (300 MHz, DMSO-d₆) δ ppm 0.93 (t, J=7.18 Hz, 6H) 2.56 (q, J=7.04 Hz, 4H) 3.31 (s, 2H) 3.44 (q, J=5.28 Hz, 2H) 4.21 (t, J=5.42 Hz, 2H) 5.64 (s, 2H) 6.77 (m, 3H) 6.91 (m, 2H) 7.01 (m, 1H) 7.75 (d, J=7.04 Hz, 1H) 9.38 (d, J=9.38 Hz, 1H) 10.21 (s, 1H).

### Example 100

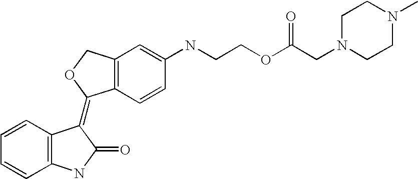

### (4-Methyl-piperazin-1-yl)-acetic acid 2-[1-(2-oxo-1,2-dihydro-indol-3-ylidene)-1,3-dihydro-isobenzofuran-5-ylamino]-ethyl ester

¹H NMR (300 MHz, DMSO-d₆) δ ppm 2.14 (s, 3H) 2.30 (brs, 4H) 2.47 (br, 4H) 3.21 (s, 2H) 3.44 (q, J=5.28 Hz, 2H) 4.22 (t, J=5.57 Hz, 2H) 5.64 (s, 2H) 6.77 (m, 3H) 6.90 (m, 2H) 7.02 (t, J=7.48 Hz, 1H) 7.75 (d, J=7.92 Hz, 1H) 9.38 (d, J=8.79 Hz, 1H) 10.21 (s, 1H).

### Example 101

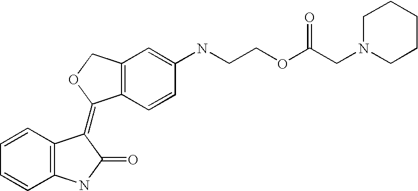

### Piperidin-1-yl-acetic acid 2-[1-(2-oxo-1,2-dihydro-indol-3-ylidene)-1,3-dihydro-isobenzofuran-5-ylamino]-ethyl ester

¹H NMR (300 MHz, DMSO-d₆) δ ppm 1.34 (m, 2H) 1.46 (m, 4H) 2.42 (m, 4H) 3.17 (s, 2H) 3.44 (q, J=5.57 Hz, 2H) 4.21 (t, J=5.42 Hz, 2H) 5.64 (s, 2H) 6.77 (m, 3H) 6.91 (m, 2H) 7.02 (t, J=7.04 Hz, 1H) 7.75 (d, J=7.33 Hz, 1H) 9.38 (d, J=8.79 Hz, 1H) 10.21 (s, 1H).

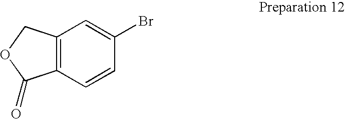

### Preparation of 5-Bromophthalide

The preparation of 5-bromophthalide is described in the literature (Safaer Hayat, Atta-ur-Rahman, M. Iqbal Choudhary, Khalid Mohammed Khan and Ernst Bayer Tetrahedron Letters, 42(2001) 1647-1649).

¹H NMR (500 MHz, CDCl₃) δ ppm 5.30 (s, 2H) 7.68 (m, 2H) 7.79 (d, J=8.79 Hz, 1H).

### Example 102

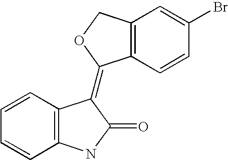

### 3-(5-Bromo-3H-isobenzofuran-1-ylidene)-1,3-dihydro-indol-2-one

To a stirred solution of oxindole (3.13 g, 23.5 mmol) in anhydrous dimethoxyethane (20 ml) under nitrogen was added 1.0M LiHMDS/THF solution (49 ml, 49 mmol). The mixture was stirred at room temperature for 10 minutes, and 5-bromophthalide (3.00 g, 14.1 mmol) was added. The reaction mixture was stirred at room temperature for 3 hours and then poured into a mixture of THF (50 ml) and 2M HCl aqueous solution (50 ml). After heated at reflux for 1 hour and then cooled to room temperature, the resulting mixture was poured into water (100 ml). The resulting solid was filtered, washed with water and dried under vacuum to give 3-(5-bromo-3H-isobenzofuran-1-ylidene)-1,3-dihydro-indol-2-one as a yellow solid (3.57 g, 77%).

¹H NMR (500 MHz, DMSO-d₆) δ ppm 5.80 (s, 2H) 6.83 (d, J=7.81 Hz, 1H) 6.97 (td, J=7.69, 1.22 Hz, 1H) 7.13 (td, J=7.69, 1.22 Hz, 1H) 7.79 (dd, J=8.30, 1.95 Hz, 1H) 7.83 (d, J=7.32 Hz, 1H) 7.92 (s, 1H) 9.58 (d, J=8.30 Hz, 1H) 10.48 (s, 1H).

### Example 103

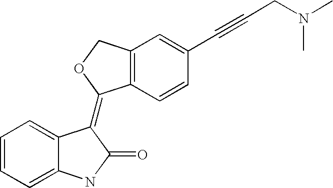

### 3-[5-(3-Dimethylamino-prop-1-ynyl)-3H-isobenzofuran-1-ylidene]-1,3-dihydro-indol-2-one

To a mixture of 3-(5-bromo-3H-isobenzofuran-1-ylidene)-1,3-dihydro-indol-2-one (467 mg, 1.42 mmol), 1-dimethylamino-2-propyne (473 mg, 5.69 mmol), copper(I) iodide (48 mg, 0.25 mmol), triethylamine (287 mg, 2.84 mmol) in DMF (5 ml), was added tetrakis(triphenylphosphine)palladium (100 mg, 0.086 mmol). The mixture was heated at 65° C. under argon for 16 hours, and then cooled to room temperature. The mixture was diluted with MeOH (100 ml). The insoluble material was removed by filtration, and the filtrate solution was concentrated and then diluted with EtOAc (200 ml). The resulting mixture was washed with brine (3×100 ml), dried over anhydrous Na₂SO₄, and concentrated to give a dark-brown residue. Purification of the residue by silica gel column chromatography, eluted with a gradient of MeOH in CHCl₃, afforded 3-[5-(3-dimethylamino-prop-1-ynyl)-3H-isobenzofuran-1-ylidene]-1,3-dihydro-indol-2-one as a brown solid (320 mg, 68%).

¹H NMR (500 MHz, DMSO-d₆) δ ppm 2.27 (s, 6H) 3.53 (s, 2H) 5.78 (s, 2H) 6.82 (d, J=7.32 Hz, 1H) 6.96 (t, J=7.57 Hz, 1H) 7.12 (t, J=7.08 Hz, 1H) 7.62 (d, J=8.30 Hz, 1H) 7.71 (s, 1H) 7.83 (d, J=7.32 Hz, 1H) 9.63 (d, J=8.30 Hz, 1H) 10.46 (s, 1H).

### Example 104

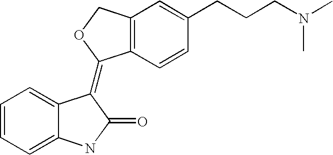

### 3-[5-(3-Dimethylamino-propyl)-3H-isobenzofuran-1-ylidene]-1,3-dihydro-indol-2-one

A mixture of 3-[5-(3-dimethylamino-prop-1-ynyl)-3H-isobenzofuran-1-ylidene]-1,3-dihydro-indol-2-one (300 mg, 0.91 mmol) and 10% palladium on carbon (60 mg) in MeOH (20 ml) was shaken under 43 psi of hydrogen for 4 hours. The catalyst was removed by filtration through celite and rinsed with MeOH. The combined filtrates were evaporated to give a crude product. The crude product was purified by silica gel column chromatography, eluted with a gradient of MeOH in CHCl₃, to give 3-[5-(3-dimethylamino-propyl)-3H-isobenzofuran-1-ylidene]-1,3-dihydro-indol-2-one as a yellow solid (70 mg, 23%).

¹H NMR (500 MHz, DMSO-d₆) δ ppm 2.02 (m, 2H) 2.78 (m, 8H) 3.06 (m, 2H) 5.80 (s, 2H) 6.83 (d, J=7.32 Hz, 1H) 6.96 (t, J=7.57 Hz, 1H) 7.11 (t, J=7.08 Hz, 1H) 7.46 (d, J=8.30 Hz, 1H) 7.53 (s, 1H) 7.83 (d, J=7.32 Hz, 1H) 9.59 (d, J=8.30 Hz, 1H) 10.41 (s, 1H).

### Example 105

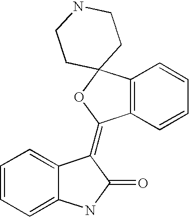

### (3E)-3-(3H-spiro[2-benzofuran-1,4′-piperidin]-3-ylidene)-1,3-dihydro-2H-indol-2-one

To a stirred solution of oxindole (139 mg, 1.04 mmol) in anhydrous DMF (5 ml) under nitrogen was added 1.0M LiHMDS/THF solution (6.0 ml, 6.0 mmol). The mixture was stirred at room temperature for 10 minutes, and then 4-spiro-[3-phthalide]piperidine hydrochloride (200 mg, 0.83 mmol) was added. The mixture was stirred at room temperature for 2.5 hours and then poured into 0.5M HCl (40 ml) with stirring. The mixture was basified with NaOH aqueous solution and extracted with CHCl₃ (2×100 ml). The combined organic layers were washed with water (2×100 ml), dried over anhydrous Na₂SO₄ and evaporated to give a crude product. Purification of the crude product by silica gel column chromatography, eluted with a gradient of MeOH in CHCl₃, produce the example 116 as a yellow solid (86 mg, 22%).

¹H NMR (500 MHz, DMSO-d₆) δ ppm 1.59 (d, J=13.18 Hz, 2H) 2.10 (m, 2H) 3.04 (m, 4H) 6.83 (d, J=7.81 Hz, 1H) 7.01 (t, J=7.57 Hz, 1H) 7.12 (t, J=7.57 Hz, 1H) 7.57 (m, 2H) 7.65 (t, J=7.32 Hz, 1H) 7.88 (d, J=7.32 Hz, 1H) 9.61 (d, J=7.81 Hz, 1H) 10.42 (s, 1H); LR MS (EI): 318 (M⁺).

### Example 106

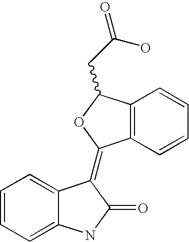

### [3-(2-Oxo-1,2-dihydro-indol-3-ylidene)-1,3-dihydro-isobenzofuran-1-yl]-acetic acid

To a solution of oxindole (0.400 g, 3.0 mmol) in DME (8.0 mL) was added 9.0 mL LHMDS (1.0 M in THF). After stirring at room temperature for 10 min, phthalide-3-acetic acid (0.519 g, 2.7 mmol) was added as a solid in one portion. The mixture was rapidly stirred for 3.5 h and then quenched into 4% HCl aqueous solution (100 mL) to give a yellow solid. The solid was filtered, rinsed with H₂O, MeOH and then 6:4/hexane:EtOAc to afford (0.105 g, 13%) of product as a yellow solid.

¹H NMR (500 MHz, DMSO-D6) δ ppm 2.73 (dd, J=16.60, 9.28 Hz, 1H) 3.30 (dd, J=16.60, 3.42 Hz, 1H) 6.24 (dd, J=9.03, 3.66 Hz, 1H) 6.83 (d, J=7.81 Hz, 1H) 6.93 (td, J=7.57, 0.98 Hz, 1H) 7.12 (td, J=7.57, 0.98 Hz, 1H) 7.59 (m, 1H) 7.68 (m, 2H) 7.80 (d, J=7.32 Hz, 1H) 9.62 (d, J=8.30 Hz, 1H) 10.44 (s, 1H) 12.68 (s, 1H).

The following Example 107 through 108 were prepared using the experiment procedure described in Example 106, but with the appropriate reagent, reaction conditions and reactant substitutions that will be readily realized by those of ordinary skill in this art without the exercise of undue experimentation

### Example 107

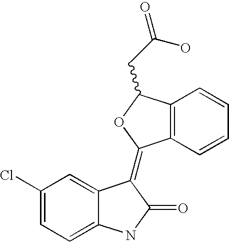

### [3-(5-Chloro-2-oxo-1,2-dihydro-indol-3-ylidene)-1,3-dihydro-isobenzofuran-1-yl]-acetic acid

¹H NMR (500 MHz, DMSO-D6) δ ppm 2.77 (dd, J=17.09, 9.28 Hz, 1H) 3.31 (obsc dd, J=3.42 Hz, 1H) 6.28 (dd, J=9.03, 3.66 Hz, 1H) 6.83 (d, J=8.30 Hz, 1H) 7.16 (dd, J=8.30, 2.44 Hz, 1H) 7.61 (m, 1H) 7.70 (m, 2H) 7.80 (d, J=1.95 Hz, 1H) 9.60 (d, J=7.81 Hz, 1H) 10.58 (s, 1H) 12.74 (s, 1H).

### Example 108

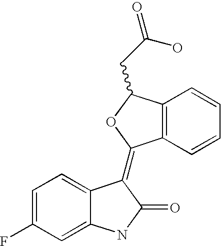

### [3-(6-Fluoro-2-oxo-1,2-dihydro-indol-3-ylidene)-1,3-dihydro-isobenzofuran-1-yl]-acetic acid

¹H NMR (500 MHz, DMSO-D6) δ ppm 2.74 (dd, J=16.60, 9.28 Hz, 1H) 3.29 (dd, J=16.60, 3.91 Hz, 1H) 6.24 (dd, J=9.03, 3.66 Hz, 1H) 6.65 (dd, J=9.28, 1.46 Hz, 1H) 6.75 (m, 1H) 7.59 (t, J=7.32 Hz, 1H) 7.68 (m, 2H) 7.77 (dd, J=8.05, 6.10 Hz, 1H) 9.56 (d, J=8.30 Hz, 1H) 10.60 (s, 1H) 12.68 (s, 1H).

### Example 109

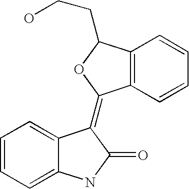

### 3-[3-(2-Hydroxy-ethyl)-3H-isobenzofuran-1-ylidene]-1,3-dihydro-indol-2-one

To a solution of [3-(2-oxo-1,2-dihydro-indol-3-ylidene)-1,3-dihydro-isobenzofuran-1-yl]-acetic acid (1.00 g, 3.25 mmol) in dioxane (100 ml) at room temperature was added dropwise 1.0M borane/THF (14.3 ml). The reaction was stirred at room temperature for 15 minutes, then heated at 65° C. for 10 minutes, and then cooled to room temperature. An additional 1.0M borane/THF (2.0 ml) was added and the mixture was heated at 65° C. for 5 minutes. Upon cooling the reaction was quenched with 4% HCl aqueous solution and then extracted with EtOAc. The combined organic layers were washed with saturated NaHCO₃ aqueous solution, brine, dried over anhydrous Na₂SO₄, and rotary evaporated to provide a yellow film. The yellow film was recrystallized from CHCl₃/hexane to give a pure yellow solid (0.466 g). The filtrate was chromatographed with 2% MeOH in CHCl₃ and then recrystallized from 1,2-dichloroethane/hexane to give an additional 0.064 g for a combined yield of 3-[3-(2-hydroxy-ethyl)-3H-isobenzofuran-1-ylidene]-1,3-dihydro-indol-2-one as a yellow solid (0.53 g, 56%).

¹H NMR (500 MHz, DMSO-D6) δ ppm 1.81 (m, 1H) 2.31 (m, 1H) 3.73 (m, 1H) 3.82 (m, 1H) 4.88 (t, J=5.13 Hz, 1H) 6.06 (dd, J=9.52, 3.17 Hz, 1H) 6.83 (d, J=7.81 Hz, 1H) 6.98 (t, J=7.57 Hz, 1H) 7.12 (m, 1H) 7.58 (m, 1H) 7.66 (m, 2H) 7.84 (d, J=7.32 Hz, 1H) 9.64 (d, J=8.30 Hz, 1H) 10.43 (s, 1H).

### Example 110

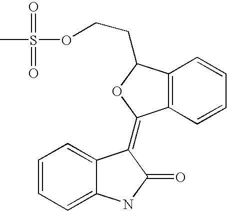

### Methanesulfonic acid 2-[3-(2-oxo-1,2-dihydro-indol-3-ylidene)-1,3-dihydro-isobenzofuran-1-yl]-ethyl ester

To a mixture of 3-[3-(2-hydroxy-ethyl)-3H-isobenzofuran-1-ylidene]-1,3-dihydro-indol-2-one (0.50 g, 1.71 mmol) and triethylamine (356 μl, 2.557 mmol) in 1,2-dichloroethane (23 ml) cooled to 0° C. was added methanesulfonyl chloride (145 μl, 1.88 mmol). After 20 minutes at 0° C. the reaction was partitioned between 0.5% HCl aqueous solution and EtOAc. The organic layer was then washed with water, brine, dried with anhydrous Na₂SO₄ Removal of the solvent led to methanesulfonic acid 2-[3-(2-oxo-1,2-dihydro-indol-3-ylidene)-1,3-dihydro-isobenzofuran-1-yl]-ethyl ester as a yellow solid (0.502 g, 79%).

¹H NMR (500 MHz, DMSO-D6) δ ppm 2.14 (m, 1H) 2.66 (m, 1H) 3.23 (s, 3H) 4.48 (m, 1H) 4.55 (m, 1H) 6.09 (dd, J=9.28, 2.93 Hz, 1H) 6.83 (d, J=7.81 Hz, 1H) 6.96 (m, 1H) 7.13 (td, J=7.57, 0.98 Hz, 1H) 7.60 (m, 1H) 7.69 (m, 2H) 7.89 (d, J=7.81 Hz, 1H) 9.65 (d, J=8.30 Hz, 1H) 10.45 (s, 1H).

### Example 111

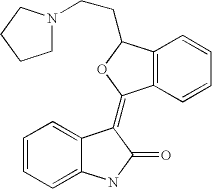

### 3-[3-(2-Pyrrolidin-1-yl-ethyl)-3H-isobenzofuran-1-ylidene]-1,3-dihydro-indol-2-one

A solution of methanesulfonic acid 2-[3-(2-oxo-1,2-dihydro-indol-3-ylidene)-1,3-dihydro-isobenzofuran-1-yl]-ethyl ester (50 mg, 0.135 mmol) and pyrrolidine (113 μl, 1.35 mmol) in dioxane (0.8 ml) was heated at 85° C. for 2 minutes and then stirred at room temperature for 4 hours. The reaction was partitioned between EtOAc and water. The EtOAc layer was washed with water, brine, dried with anhydrous Na₂SO₄ and rotary evaporated to give brown oil. The brown oil was chromatographed (CHCl₃ to 2.5% MeOH/CHCl₃ gradient) and then recrystallized from EtOAc/hexane to produce 3-[3-(2-pyrrolidin-1-yl-ethyl)-3H-isobenzofuran-1-ylidene]-1,3-dihydro-indol-2-one as a yellow solid (25 mg, 54%).

¹H NMR (500 MHz, DMSO-D6) δ ppm 1.69 (br s, 4H) 1.91 (m, 1H) 2.36 (m, 1H) 2.52 (obsc m, 5H) 2.75 (m, 1H) 6.04 (dd, J=8.30, 3.42 Hz, 1H) 6.83 (d, J=7.81 Hz, 1H) 6.98 (t, J=7.57 Hz, 1H) 7.12 (m, 1H) 7.57 (m, 1H) 7.67 (m, 2H) 7.84 (d, J=7.32 Hz, 1H) 9.63 (d, J=7.81 Hz, 1H) 10.43 (s, 1H).

### Example 112

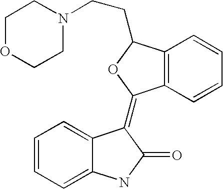

### 3-[3-(2-Morpholin-4-yl-ethyl)-3H-isobenzofuran-1-ylidene]-1,3-dihydro-indol-2-one

A solution of methanesulfonic acid 2-[3-(2-oxo-1,2-dihydro-indol-3-ylidene)-1,3-dihydro-isobenzofuran-1-yl]-ethyl ester (50 mg, 0.135 mmol) and morpholine (118 μl, 1.35 mmol) in dioxane (0.8 ml) was heated at 70° C. for 40 minutes and then at 60° C. for 18 hours. The reaction was partitioned between EtOAc and water. The EtOAc layer was washed with water, brine, dried with anhydrous Na₂SO₄ and rotary evaporated to give a yellow film. The yellow film was chromatographed (CHCl₃ to 2% MeOH/CHCl₃ gradient) to give 3-[3-(2-morpholin-4-yl-ethyl)-3H-isobenzofuran-1-ylidene]-1,3-dihydro-indol-2-one as a yellow solid (37 mg, 76%).

¹H NMR (500 MHz, CDCl₃) δ ppm 2.02 (m, 1H) 2.33 (m, 1H) 2.49 (m, 4H) 2.59 (m, 1H) 2.70 (m, 1H) 3.69 (t, J=4.88 Hz, 4H) 5.95 (dd, J=8.30, 3.91 Hz, 1H) 6.88 (d, J=7.81 Hz, 1H) 7.06 (t, J=7.32 Hz, 1H) 7.17 (m, 1H) 7.40 (m, 1H) 7.56 (m, 2H) 7.60 (s, 1H) 7.94 (d, J=7.81 Hz, 1H) 9.71 (d, J=6.83 Hz, 1H).

### Example 113

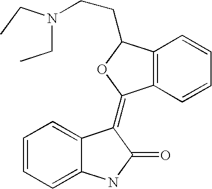

### 3-[3-(2-Diethylamino-ethyl)-3H-isobenzofuran-1-ylidene]-1,3-dihydro-indol-2-one

A solution of methanesulfonic acid 2-[3-(2-oxo-1,2-dihydro-indol-3-ylidene)-1,3-dihydro-isobenzofuran-1-yl]-ethyl ester (50 mg, 0.135 mmol) and diethylamine (279 μl, 2.7 mmol) in THF (0.8 ml) was heated at 60° C. After 3 hours, dioxane (0.3 ml) was added to give a solution and the reaction continued at 60° C. for 24 h. The reaction was partitioned between EtOAc and water. The EtOAc layer was washed with water, brine, dried with anhydrous Na₂SO₄ and rotary evaporated to give brown oil. The brown oil was chromatographed (CHCl₃ to 2% MeOH/CHCl₃ gradient) to yield 3-[3-(2-diethylamino-ethyl)-3H-isobenzofuran-1-ylidene]-1,3-dihydro-indol-2-one as a yellow-green solid (33 mg, 71%).

¹H NMR (500 MHz, CDCl₃) δ ppm 1.06 (t, J=7.08 Hz, 6H) 1.93 (m, 1H) 2.27 (m, 1H) 2.59 (m, 4H) 2.72 (m, 1H) 2.85 (m, 1H) 5.93 (dd, J=8.79, 3.42 Hz, 1H) 6.87 (d, J=7.81 Hz, 1H) 7.05 (m, 1H) 7.16 (m, 1H) 7.40 (m, 1H) 7.56 (m, 2H) 7.60 (s, 1H) 7.97 (d, J=6.83 Hz, 1H) 9.71 (m, 1H).

### Example 114

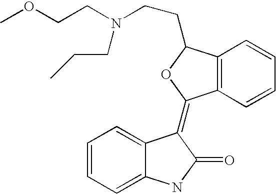

### 3-(3-{2-[(2-Methoxy-ethyl)-propyl-amino]-ethyl}-3H-isobenzofuran-1-ylidene)-1,3-dihydro-indol-2-one

A solution of methanesulfonic acid 2-[3-(2-oxo-1,2-dihydro-indol-3-ylidene)-1,3-dihydro-isobenzofuran-1-yl]-ethyl ester (50 mg, 0.135 mmol) and N-(2-methoxyethyl)-N-propylamine (191 μl, 1.35 mmol) in dioxane (0.8 ml) was heated at 75° C. for 19 hours. The reaction was partitioned between EtOAc and water. The EtOAc layer was washed with water, brine, dried with anhydrous Na₂SO₄ and rotary evaporated to give brown oil. The brown oil was chromatographed with CHCl₃ to give 3-(3-{2-[(2-methoxy-ethyl)-propyl-amino]-ethyl}-3H-isobenzofuran-1-ylidene)-1,3-dihydro-indol-2-one as a yellow-brown film (11 mg, 20%).

¹H NMR (500 MHz, CDCl₃) δ ppm 0.91 (t, J=7.32 Hz, 3H) 1.51 (m, 2H) 1.89 (m, 1H) 2.27 (m, 1H) 2.51 (m, 2H) 2.68 (m, 1H) 2.77 (m, 2H) 2.93 (m, 1H) 3.34 (s, 3H) 3.48 (m, 2H) 5.98 (dd, J=9.03, 3.17 Hz, 1H) 6.88 (d, J=7.81 Hz, 1H) 7.05 (t, J=7.57 Hz, 1H) 7.16 (m, 1H) 7.40 (d, J=7.32 Hz, 1H) 7.55 (m, 2H) 7.87 (s, 1H) 7.96 (d, J=7.32 Hz, 1H) 9.72 (m, 1H).

### Example 115

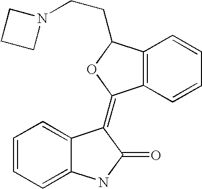

### 3-[3-(2-Azetidin-1-yl-ethyl)-3H-isobenzofuran-1-ylidene]-1,3-dihydro-indol-2-one

To a slurry of azetidine hydrochloride (126 mg, 1.35 mmol) in THF (11.0 ml) was added a scoop of Aberlite-IRA-93 ion exchange resin, which was shaken 3 minutes to give a solution, dried with anhydrous Na₂SO₄ and filtered to remove resin. Then methanesulfonic acid 2-[3-(2-oxo-1,2-dihydro-indol-3-ylidene)-1,3-dihydro-isobenzofuran-1-yl]-ethyl ester (50.0 mg, 0.135 mmol) was added to the amine solution and heated at 60° C. for 18 hours (no reaction had occurred). After standing at room temperature for 2 days, to the mixture were addded dioxane (0.3 ml), azetidine hydrochloride (63 mg, 0.675 mmol), and triethylamine (132 μl, 0.945 mmol). The resulting mixture was stirred at room temperature for 3 days, and heated at 60° C. for 24 hours. The reaction was partitioned between water and EtOAc. The EtOAc layer was extracted with 4% HCl aqueous solution, and then the aqueous layer was basified with saturated NaHCO₃ aqueous solution. The basified aqueous layer was extracted with EtOAc. The organic layer was washed with brine, dried over anhydrous Na₂SO₄ and rotary evaporated to give a film. The film was chromatographed (2.5% to 5% MeOH/CHCl₃ gradient) to provide 3-[3-(2-azetidin-1-yl-ethyl)-3H-isobenzofuran-1-ylidene]-1,3-dihydro-indol-2-one as a yellow film (8.7 mg, 19%).

¹H NMR (500 MHz, CDCl₃) δ ppm 1.86 (m, 1H) 2.13 (m, 3H) 2.57 (m, 1H) 2.74 (m, 1H) 3.24 (m, 4H) 5.90 (dd, J=8.30, 3.91 Hz, 1H) 6.88 (d, J=7.81 Hz, 1H) 7.08 (t, J=7.57 Hz, 1H) 7.17 (t, J=7.57 Hz, 1H) 7.39 (m, 1H) 7.55 (m, 2H) 7.92 (s, 1H) 7.97 (d, J=7.32 Hz, 1H) 9.71 (m, 1H).

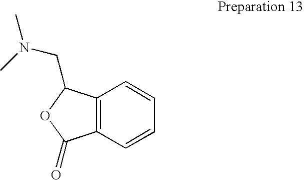

### Preparation of 3-Dimethylaminomethyl-3H-isobenzofuran-1-one

To a solution of phthalide (0.50 g, 3.73 mmol) in THF (13.0 ml) at −78° C. was added 1.0M LiHMDS in THF (3.9 ml). The reaction was stirred for 10 minutes, and then N,N-dimethylmethyleneammonium iodide (0.76 g, 4.10 mmol) was added in one portion. The resulting mixture was continuously stirred at −78° C. for another 15 minutes. The mixture was then allowed to warm to 0° C. and then quenched into very dilute HCl aqueous solution. The aqueous layer was adjusted to acidic pH with 4% HCl aqueous solution and washed with EtOAc. The aqueous layer was basified with saturated NaHCO₃ aqueous solution and extracted with EtOAc. The combined organic layers were washed with water, brine, dried over anhydrous Na₂SO₄ and rotary evaporated to a light yellow oil. The oil was taken up in CHCl₃, filtered to remove an insoluble impurity, and then chromatographed through silica gel column (CHCl₃ to 2.5% MeOH/CHCl₃ gradient) to afford 3-dimethylaminomethyl-3H-isobenzofuran-1-one as a clear oil (0.24 g, 33%).

¹H NMR (500 MHz, CDCl₃) δ ppm 2.41 (s, 6H) 2.67 (dd, J=13.18, 7.32 Hz, 1H) 2.85 (dd, J=13.43, 4.64 Hz, 1H) 5.55 (dd, J=7.57, 4.64 Hz, 1H) 7.54 (m, 2H) 7.67 (t, J=7.81 Hz, 1H) 7.91 (m, 1H).

### Example 116

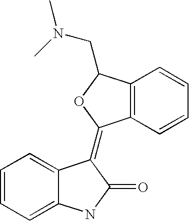

### 3-(3-Dimethylaminomethy-3H-isobenzofuran-1-ylidene)-1,3-dihydro-indol-2-one

To a solution of oxindole (205 mg, 1.54 mmol) in dimethoxyethane (4.0 ml) at room temperature was added 1.0M LiHMDS in THF (1.54 ml). After stirring for 10 minutes at room temperature, 3-dimethylaminomethyl-3H-isobenzofuran-1-one (235 mg, 1.23 mmol) in dimethoxyethane (0.5 ml) was added and the mixture was stirred rapidly for 1 hour. Additional 1.0M LiHMDS/THF (1.54 ml) was added. After 2 hours the reaction was quenched into 10% HCl (50 ml) and 25 ml water added. The aqueous layer was washed with EtOAc and then basified with saturated NaHCO₃ aqueous solution. The aqueous layer was extracted with EtOAc. The EtOAc layer was washed with brine, dried over anhydrous Na₂SO₄, and rotary evaporated to provide a yellow solid. The yellow solid was chromatographed silica gel column (CHCl₃ to 2.5% MeOH/CHCl₃ gradient) and then recrystallized from MeOH to give 3-(3-dimethylaminomethy-3H-isobenzofuran-1-ylidene)-1,3-dihydro-indol-2-one as yellow needles (92 mg, 24%).

¹H NMR (500 MHz, CDCl₃) δ ppm 2.48 (s, 6H) 2.78 (dd, J=13.67, 7.32 Hz, 1H) 2.96 (dd, J=13.67, 3.91 Hz, 1H) 5.91 (dd, J=7.08, 4.15 Hz, 1H) 6.87 (d, J=7.32 Hz, 1H) 7.05 (m, 1H) 7.16 (m, 1H) 7.49 (m, 1H) 7.56 (m, 2H) 7.68 (s, 1H) 8.00 (d, J=7.81 Hz, 1H) 9.72 (m, 1H).

### Example 117

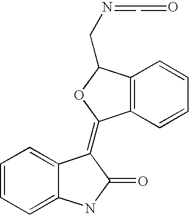

### 3-(3-Isocyanatomethyl-3H-isobenzofuran-1-ylidene)-1,3-dihydro-indol-2-one

To a mixture of [3-(2-oxo-1,2-dihydro-indol-3-ylidene)-1,3-dihydro-isobenzofuran-1-yl]-acetic acid (200 mg, 0.65 mmol) in THF (8.0 ml) at room temperature was added 2.5M n-BuLi in hexane (272 μl), and the yellow-brown slurry was stirred for 10 minutes. After cooling to 0° C., 2.0M oxalyl chloride in CH₂Cl₂ (325 μl) was added. The clear orange solution was stirred for 25 minutes, and then sodium azide (51 mg, 0.78 mmol) in water (0.5 ml) was added, followed by addition of water (1 ml). After 20 minutes, the reaction was partitioned between dilute NaHCO₃ aqueous solution and CH₂Cl₂. The CH₂Cl₂ layer was washed with brine and dried with anhydrous Na₂SO₄ to give a yellow solution. The solution was diluted with toluene (200 ml), and then rotary evaporated to 100 ml volume. The toluene solution was heated at 90° C. for 40 minutes, cooled to room temperature, and then rotary evaporated to give 3-(3-isocyanatomethyl-3H-isobenzofuran-1-ylidene)-1,3-dihydro-indol-2-one as a yellow oily solid (130 mg, 66%).

¹H NMR (500 MHz, CDCl₃) δ ppm 3.77 (dd, J=13.91, 6.10 Hz, 1H) 3.90 (m, 1H) 5.91 (dd, J=6.10, 3.91 Hz, 1H) 6.88 (d, J=7.57 Hz, 1H) 7.08 (t, J=7.57 Hz, 1H) 7.19 (m, 1H) 7.43 (dd, J=4.52, 3.78 Hz, 1H) 7.61 (m, 2H) 7.78 (br s, 1H) 7.99 (d, J=7.57 Hz, 1H) 9.72 (m, 1H).

### Example 118

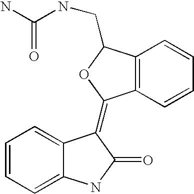

### [3-(2-Oxo-1,2-dihydro-indol-3-ylidene)-1,3-dihydro-isobenzofuran-1-ylmethyl]-urea

To a solution of 3-(3-Isocyanatomethyl-3H-isobenzofuran-1-ylidene)-1,3-dihydro-indol-2-one (65 mg, 0.21 mmol) in THF (4 ml) at room temperature was added concentrated NH₄OH (2 ml). The reaction was stirred 5 minutes and then partitioned between EtOAc and water. The organic layer was washed with very dilute HCl, brine, dried with anhydrous Na₂SO₄ and rotary evaporated to obtain a solid. The solid was recrystallized from EtOAc/MeOH to give [3-(2-oxo-1,2-dihydro-indol-3-ylidene)-1,3-dihydro-isobenzofuran-1-ylmethyl]-urea as a yellow solid (46 mg, 68%).

¹H NMR (500 MHz, DMSO-D6) δ ppm 3.57 (m, 1H) 3.75 (m, 1H) 5.57 (s, 2H) 5.97 (t, J=4.88 Hz, 1H) 6.15 (t, J=5.86 Hz, 1H) 6.83 (d, J=7.32 Hz, 1H) 6.98 (t, J=7.57 Hz, 1H) 7.12 (t, J=7.57 Hz, 1H) 7.59 (t, J=7.32 Hz, 1H) 7.65 (m, 2H) 7.91 (d, J=7.32 Hz, 1H) 9.64 (d, J=7.81 Hz, 1H) 10.43 (s, 1H).

### Example 119

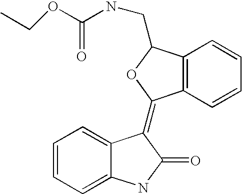

### [3-(2-Oxo-1,2-dihydro-indol-3-ylidene)-1,3-dihydro-isobenzofuran-1-ylmethyl]-carbamic acid ethyl ester

A solution of 3-(3-isocyanatomethyl-3H-isobenzofuran-1-ylidene)-1,3-dihydro-indol-2-one (74 mg, 0.244 mmol) in anhydrous EtOH (4 ml) was heated at 75° C. for 17 hours. After cooling to room temperature, the yellow precipitate was removed by filtration and the filtrate was evaporated. The filtrate sample was chromatographed with CHCl₃ to afford [3-(2-oxo-1,2-dihydro-indol-3-ylidene)-1,3-dihydro-isobenzofuran-1-ylmethyl]-carbamic acid ethyl ester as a yellow solid (18 mg, 22%).

¹H NMR (500 MHz, CDCl₃) δ ppm 1.20 (t, J=7.08 Hz, 3H) 3.59 (m, 1H) 3.98 (m, 1H) 4.09 (q, J=6.83 Hz, 2H) 5.00 (t, J=6.35 Hz, 1H) 5.93 (dd, J=5.37, 3.42 Hz, 1H) 6.88 (d, J=7.81 Hz, 1H) 7.07 (t, J=7.57 Hz, 1H) 7.18 (t, J=7.57 Hz, 1H) 7.50 (d, J=6.83 Hz, 1H) 7.57 (m, 2H) 7.85 (s, 1H) 7.93 (d, J=7.81 Hz, 1H) 9.69 (d, J=7.32 Hz, 1H).

### Example 120

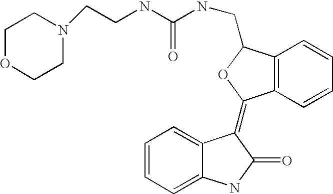

### 1-(2-Morpholin-4-yl-ethyl)-3-[3-(2-oxo-1,2-dihydro-indol-3-ylidene)-1,3-dihydro-isobenzofuran-1-ylmethyl]-urea

A solution of 3-(3-isocyanatomethyl-3H-isobenzofuran-1-ylidene)-1,3-dihydro-indol-2-one (70 mg, 0.23 mmol) and 4-(2-aminoethyl)morpholine (90.0 μl, 0.69 mmol) in THF (2.0 ml) was stirred at room temperature for 25 minutes. The reaction was heated for 5 minutes at 60° C. and then partitioned between EtOAc and water. The organic layer was washed with saturated NaHCO₃ aqueous solution, and water, and then extracted with 4% HCl aqueous solution. The combined aqueous layers were basified with saturated NaHCO₃ aqueous solution and extracted with EtOAc. The combined organic layers were washed with brine, dried with anhydrous Na₂SO₄, and rotary evaporated to a yellow foam. The yellow foam was chromatographed through silica gel column (CHCl₃ to 5% MeOH/CHCl₃ gradient) to afford 1-(2-morpholin-4-yl-ethyl)-3-[3-(2-oxo-1,2-dihydro-indol-3-ylidene)-1,3-dihydro-isobenzofuran-1-ylmethyl]-urea as a yellow foam (18 mg, 18%).

¹H NMR (500 MHz, CDCl₃) δ ppm 2.31 (m, 6H) 3.21 (m, 2H) 3.48 (t, J=4.39 Hz, 4H) 3.89 (m, 1H) 4.01 (m, 1H) 5.37 (t, J=4.88 Hz, 1H) 5.66 (s, 1H) 5.94 (t, J=3.91 Hz, 1H) 6.66 (d, J=7.81 Hz, 1H) 7.01 (m, 1H) 7.08 (m, 1H) 7.23 (m, 1H) 7.48 (m, 2H) 7.86 (s, 1H) 7.92 (d, J=7.32 Hz, 1H) 9.35 (d, J=8.30 Hz, 1H).

### Example 121

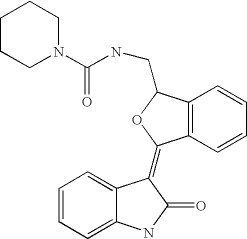

### Piperidine-1-carboxylic acid[3-(2-oxo-1,2-dihydro-indol-3-ylidene)-1,3-dihydro-isobenzofuran-1-ylmethyl]-amide

A solution of 3-(3-isocyanatomethyl-3H-isobenzofuran-1-ylidene)-1,3-dihydro-indol-2-one (95 mg, 0.31 mmol) and piperidine (92.8 μl, 0.94 mmol) in THF (2.0 ml) was stirred at room temperature for 4 hours. The reaction was partitioned between EtOAc and water. The organic layer was washed with dilute HCl aqueous solution, water, saturated NaHCO₃ aqueous solution, water, brine, dried with anhydrous Na₂SO₄, and rotary evaporated to a brown film. The brown film was chromatographed through silica gel column (CHCl₃ to 4% MeOH/CHCl₃ gradient) to produce piperidine-1-carboxylic acid[3-(2-oxo-1,2-dihydro-indol-3-ylidene)-1,3-dihydro-isobenzofuran-1-ylmethyl]-amide as a yellow solid (30 mg, 25%).

¹H NMR (500 MHz, CDCl₃) δ ppm 1.50 (m, 4H) 1.59 (m, 2H) 3.30 (m, 4H) 3.50 (m, 1H) 4.09 (m, 1H) 4.89 (t, J=5.86 Hz, 1H) 5.99 (dd, J=7.32, 3.42 Hz, 1H) 6.90 (d, J=7.81 Hz, 1H) 7.04 (t, J=7.57 Hz, 1H) 7.17 (t, J=7.57 Hz, 1H) 7.55 (m, 3H) 7.93 (d, J=7.32 Hz, 1H) 8.15 (s, 1H) 9.68 (m, 1H).

### Example 122

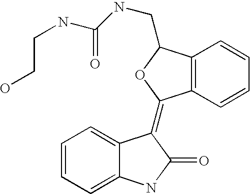

### 1-(2-Hydroxy-ethyl)-3-[3-(2-oxo-1,2-dihydro-indol-3-ylidene)-1,3-dihydro-isobenzofuran-1-ylmethyl]-urea

A solution of 3-(3-isocyanatomethyl-3H-isobenzofuran-1-ylidene)-1,3-dihydro-indol-2-one (75 mg, 0.24 mmol) and 2-hydroxyethylamine (44.4 μl, 0.74 mmol) in THF (2.0 ml) was stirred at room temperature for 25 minutes and then heated at 60° C. for 5 minutes. The reaction was partitioned between EtOAc and water. The organic layer was washed with water, brine, dried with anhydrous Na₂SO₄, and rotary evaporated to result in a brown-yellow solid. The brown-yellow solid was chromatographed through silica gel column (CHCl₃ to 5% MeOH/CHCl₃ gradient) to give 1-(2-hydroxy-ethyl)-3-[3-(2-oxo-1,2-dihydro-indol-3-ylidene)-1,3-dihydro-isobenzofuran-1-ylmethyl]-urea as a yellow solid (18 mg, is 20%).

¹H NMR (500 MHz, DMSO-D6) δ ppm 3.02 (m, 2H) 3.32 (obsc m, 2H) 3.56 (m, 1H) 3.77 (m, 1H) 4.63 (t, J=5.13 Hz, 1H) 5.96 (dd, J=5.86, 4.39 Hz, 1H) 6.12 (t, J=5.61 Hz, 1H) 6.21 (t, J=5.86 Hz, 1H) 6.83 (d, J=7.81 Hz, 1H) 6.97 (m, 1H) 7.12 (td, J=7.57, 0.98 Hz, 1H) 7.62 (m, 3H) 7.90 (d, J=7.81 Hz, 1H) 9.64 (d, J=7.81 Hz, 1H) 10.43 (s, 1H).

### Example 123

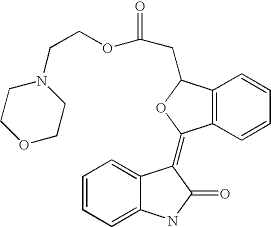

### [3-(2-Oxo-1,2-dihydro-indol-3-ylidene)-1,3-dihydro-isobenzofuran-1-yl]-acetic acid 2-morpholin-4-yl-ethyl ester

To a slurry of [3-(2-oxo-1,2-dihydro-indol-3-ylidene)-1,3-dihydro-isobenzofuran-1-yl]-acetic acid (100 mg, 0.32 mmol) in THF (4.0 ml) at room temperature was added 2.5M n-BuLi in hexane (136 μl), and the reaction was stirred for 10 minutes. After the reaction was cooled to 0° C., oxalyl chloride (163 μl, 0.32 mmol) was added. After stirring for 20 minutes, 4-(2-hydroxyethyl)morpholine (118 μl, 0.976 mmol) was added. The reaction was stirred for 5 minutes at 0° C., and then at room temperature for 20 minutes. The reaction was partitioned between EtOAc and saturated NaHCO₃. The EtOAc layer was washed with brine, dried with anhydrous Na₂SO₄ and rotary evaporated to afford an orange oil. The orange oil was chromatographed through silica gel column, eluted with CHCl₃, to give [3-(2-oxo-1,2-dihydro-indol-3-ylidene)-1,3-dihydro-isobenzofuran-1-yl]-acetic acid 2-morpholin-4-yl-ethyl ester as a yellow foam (43 mg, 32%).

¹H NMR (500 MHz, CDCl₃) δ ppm 2.49 (m, 4H) 2.65 (t, J=5.86 Hz, 2H) 2.94 (m, 1H) 3.03 (m, 1H) 3.67 (m, 4H) 4.35 (t, J=5.86 Hz, 2H) 6.23 (dd, J=7.81, 5.37 Hz, 1H) 6.87 (d, J=7.81 Hz, 1H) 7.03 (t, J=7.57 Hz, 1H) 7.16 (t, J=7.57 Hz, 1H) 7.45 (m, 1H) 7.57 (m, 2H) 7.70 (s, 1H) 7.90 (d, J=7.32 Hz, 1H) 9.70 (m, 1H).

### Example 124

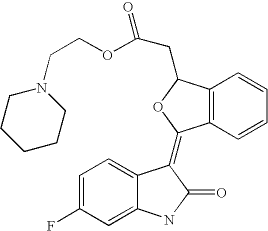

### [3-(6-Fluoro-2-oxo-1,2-dihydro-indol-3-ylidene)-1,3-dihydro-isobenzofuran-1-yl]-acetic acid 2-piperidin-1-yl-ethyl ester

To a slurry of [3-(6-fluoro-2-oxo-1,2-dihydro-indol-3-ylidene)-1,3-dihydro-isobenzofuran-1-yl]-acetic acid (449 mg, 1.38 mmol) in THF (20.0 ml) at room temperature was added 2.5M n-BuLi/Hexane (580 μl), and the reaction was stirred for 10 minutes. After cooling to 0° C., oxalyl chloride (690 μl, 1.38 mmol) was added. After 20 minutes, 4-(2-hydroxyethyl)morpholine (550 μl, 4.14 mmol) was added. The mixture was stirred for 5 minutes at 0° C., and then at room temperature for 1 hour. The reaction was partitioned between EtOAc and water. The organic layer was washed with saturated NaHCO₃ aqueous solution, brine, dried with anhydrous Na₂SO₄ and rotary evaporated to produce a brown-yellow foam. The brown-yellow foam was chromatographed through silica gel column (CHCl₃ to 2.5% MeOH/CHCl₃ gradient) to afford a yellow solid. The solid was dissolved in hot EtOAc/Hexane, allowed to cool to room temperature, and filtered to remove fluffy precipitate. The filtrate was cooled in refrigerator after concentrating the solution. The precipitate was filtered to give [3-(6-fluoro-2-oxo-1,2-dihydro-indol-3-ylidene)-1,3-dihydro-isobenzofuran-1-yl]-acetic acid 2-piperidin-1-yl-ethyl ester as a yellow solid (200 mg, 33%).

¹H NMR (500 MHz, CDCl₃) δ ppm 1.43 (m, 2H) 1.57 (m, 4H) 2.45 (br s, 4H) 2.64 (t, J=6.10 Hz, 2H) 2.92 (dd, J=16.35, 8.06 Hz, 1H) 3.01 (m, 1H) 4.35 (t, J=5.98 Hz, 2H) 6.21 (dd, J=7.93, 5.25 Hz, 1H) 6.61 (dd, J=8.79, 2.20 Hz, 1H) 6.72 (m, 1H) 7.45 (m, 1H) 7.57 (m, 2H) 7.82 (dd, J=8.54, 5.61 Hz, 1H) 8.12 (br s, 1H) 9.64 (m, 1H).

### Example 125

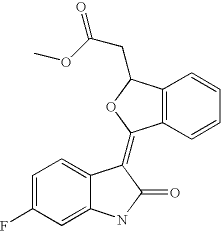

### [3-(6-Fluoro-2-oxo-1,2-dihydro-indol-3-ylidene)-1,3-dihydro-isobenzofuran-1-yl]-acetic acid methyl ester

To a slurry of [3-(6-fluoro-2-oxo-1,2-dihydro-indol-3-ylidene)-1,3-dihydro-isobenzofuran-1-yl]-acetic acid (300 mg, 0.922 mmol) in a mixture of dioxane (15 ml) and MeOH (7 ml) was added 2.0M (trimethylsilyl)diazomethane/Hexanes (507 μl) and the resultant solution was stirred for 15 minutes at room temperature. The solvent was rotary evaporated and the residue was partitioned between EtOAc and saturated NaHCO₃. The organic layer (combined a EtOAc solution from a previous 100 mg (0.307 mmol) scale test reaction) was then washed with dilute HCl aqueous solution, water, brine, dried with anhydrous Na₂SO₄, and rotary evaporated to a yellow solid. The solid was triturated with 30% EtOAc in hexane to yield [3-(6-fluoro-2-oxo-1,2-dihydro-indol-3-ylidene)-1,3-dihydro-isobenzofuran-1-yl]-acetic acid methyl ester as a bright yellow solid (388 mg, 93%).

¹H NMR (500 MHz, DMSO-D6) δ ppm 2.88 (dd, J=16.60, 8.79 Hz, 1H) 3.40 (dd, J=16.60, 3.91 Hz, 1H) 3.71 (s, 3H) 6.26 (dd, J=9.03, 3.66 Hz, 1H) 6.65 (dd, J=9.28, 2.44 Hz, 1H) 6.79 (m, 1H) 7.60 (m, 1H) 7.68 (m, 2H) 7.73 (dd, J=8.54, 5.61 Hz, 1H) 9.56 (d, J=7.81 Hz, 1H) 10.61 (s, 1H).

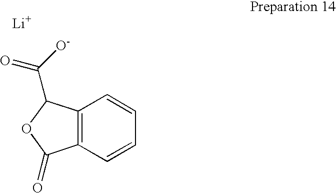

### Preparation of Lithium 3-oxo-1,3-dihydro-2-benzofuran-1-carboxylate

To a solution of phthalide (2.0 g, 14.9 mmol) in THF (60.0 ml) at −78° C. was added 1.0M LiHMDS/THF (15.7 ml) over 10 minutes. The reaction was stirred for 15 minutes at −78° C. and then the ice bath was removed. The reaction was quenched into dry ice using a cannula and then allowed to warm to room temperature. After adding 40 ml hexane, the cloudy mixture was rotary evaporated and chased with hexane to give lithium 3-oxo-1,3-dihydro-2-benzofuran-1-carboxylate as a yellow solid (2.86 g, 100%).

¹H NMR (500 MHz, DMSO-D6) δ ppm 5.57 (s, 1H) 7.49 (t, J=7.57 Hz, 1H) 7.66 (t, J=7.57 Hz, 1H) 7.72 (t, J=7.81 Hz, 2H).

### Example 126

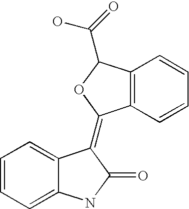

### 3-(2-Oxo-1,2-dihydro-indol-3-ylidene)-1,3-dihydro-isobenzofuran-1-carboxylic acid

To a solution of oxindole (1.10 g, 8.27 mmol) in dimethoxyethane (30.0 ml) at room temperature was added 1.0M LiHMDS/THF (17.0 ml). The mixture was stirred for 10 minutes at room temperature, and lithium 3-oxo-1,3-dihydro-2-benzofuran-1-carboxylate (1.37 g, 7.44 mmol) was added in one portion. After the reaction was rapidly stirred at room temperature for 18 hours, it was quenched into 4% HCl aqueous solution (200 mL), and the mixture was stirred 5 minutes. The aqueous layer was extracted with EtOAc. The combined organic layers were washed with water and then extracted with saturated NaHCO₃ aqueous solution. The aqueous layer was acidified with 4% HCl aqueous solution, and extracted with EtOAc. The combined organic layers were washed with brine, dried with anhydrous Na₂SO₄, and rotary evaporated to obtain a yellow solid. The yellow solid was triturated at room temperature with CHCl₃, and then triturated with hot isopropanol to give 3-(2-oxo-1,2-dihydro-indol-3-ylidene)-1,3-dihydro-isobenzofuran-1-carboxylic acid as a yellow solid (287 mg, 13%).

¹H NMR (500 MHz, DMSO-D6) δ ppm 6.54 (s, 1H) 6.85 (d, J=7.57 Hz, 1H) 7.00 (t, J=7.57 Hz, 1H) 7.15 (t, J=7.57 Hz, 1H) 7.65 (m, 1H) 7.71 (m, 2H) 7.89 (d, J=7.57 Hz, 1H) 9.63 (d, J=7.81 Hz, 1H) 10.51 (s, 1H) 13.90 (s, 1H).

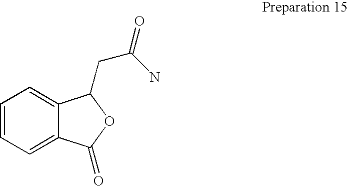

### Preparation of 2-(3-oxo-1,3-dihydro-2-benzofuran-1-yl)acetamide

A solution of (3-oxo-1,3-dihydro-isobenzofuran-1-yl)-acetic acid (5.0 g, 26 mmol) in thionyl chloride (10 ml) was heated at 75° C. for 15 minutes. The excess thionyl chloride was removed under vacuum to give a red oil. The red oil was dissolved in CH₂Cl₂ (10 ml) and then the solution was slowly added to concentrated NH₄OH (40 ml) at 0° C. The resultant solid was filtered and washed with water and hexane, follwed by addition of toluene. Removal of the solvent led to 2-(3-oxo-1,3-dihydro-2-benzofuran-1-yl)acetamide as a cream solid (4.2 g, 84%).

¹H NMR (300 MHz, DMSO-D6) δ ppm 2.56 (dd, J=15.39, 8.65 Hz, 1H) 2.86 (dd, J=15.24, 4.98 Hz, 1H) 5.91 (dd, J=8.35, 4.84 Hz, 1H) 7.09 (br s, 1H) 7.49 (br s, 1H) 7.61 (t, J=7.48 Hz, 1H) 7.70 (d, J=7.62 Hz, 1H) 7.81 (m, 2H).

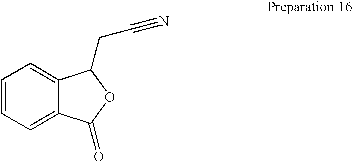

### Preparation of (3-oxo-1,3-dihydro-2-benzofuran-1-yl)acetonitrile

To a mixture of P₂O₅ (4.0 g, 28.2 mmol) in xylenes (20 ml) was added 2-(3-oxo-1,3-dihydro-2-benzofuran-1-yl)acetamide (2.0 g, 10.5 mmol) and the reaction was heated at 140° C. for 5 minutes. The mixture was filtered while warm to give the xylene filtrate, and a brown gummy solid. The brown gummy solid was triturated with toluene to give a light yellow solution. The xylene filtrate was combined with the yellow solution, and diluted with hexane. Upon cooling at 0° C., a white precipitate formed. The precipitate was separated to give solid A and filtrate A.

The solid A was partitioned between a mixture of 4% HCl aqueous solution and EtOAc. The EtOAc layer was washed with water, brine, and dried over anhydrous Na₂SO₄ Removal of the solvent led to white solid B (0.18 g).

The above brown gummy solid was partitioned between water and EtOAc. The EtOAc layer was separated, and combined with filtrate A. The combined filtrates were washed with water, brine, dried with anhydrous Na₂SO₄. Evaporation of the filtrates provided solid C, which was chromatographed through silica gel column, eluted with 50% EtOAc in hexane to provide white solid D (0.90 g).

The solid B and solid D were combined to afford (3-oxo-1,3-dihydro-2-benzofuran-1-yl)acetonitrile (1.08 g, 60%).

¹H NMR (300 MHz, CHLOROFORM-D) δ ppm 2.96 (dd, J=16.71, 6.74 Hz, 1H) 3.11 (m, 1H) 5.68 (m, 1H) 7.66 (m, 2H) 7.78 (m, 1H) 7.97 (d, J=7.62 Hz, 1H).

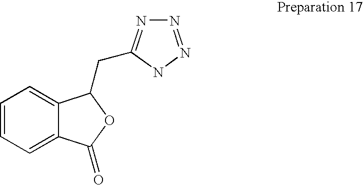

### Preparation of 3-(1H-tetrazol-5-ylmethyl)-2-benzofuran-1(3H)-one

A solution of (3-oxo-1,3-dihydro-2-benzofuran-1-yl)acetonitrile (890 mg, 5.14 mmol) and azidotributyltin (1.97 ml, 7.20 mmol) in dioxane (4.0 ml) was heated at 95° C. for 17 hours. The reaction was partitioned between EtOAc and saturated NaHCO₃ aqueous solution, and then washed with saturated NaHCO₃ aqueous solution. The aqueous layers were combined, washed with EtOAc, then acidified with 10% HCl aqueous solution. The acidic aqueous layer was extracted with EtOAc. The combined organic layers were then washed with brine, dried with anhydrous Na₂SO₄, and rotary evaporated to obtain a yellow oil. The oil was dissolved in CHCl₃/Hexane and allowed to crystallize, leading to 3-(1H-tetrazol-5-ylmethyl)-2-benzofuran-1(3H)-one as a white solid (913 mg, 82%).

¹H NMR (300 MHz, DMSO-D6) δ ppm 3.49 (dd, J=15.68, 7.48 Hz, 1H) 3.83 (dd, J=15.68, 4.25 Hz, 1H) 6.06 (dd, J=7.48, 4.25 Hz, 1H) 7.61 (t, J=7.48 Hz, 1H) 7.74 (d, J=7.62 Hz, 1H) 7.81 (t, J=7.04 Hz, 2H).

### Example 127

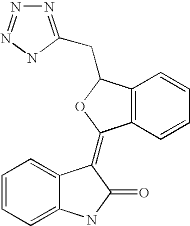

### (3E)-3-[3-(1H-tetrazol-5-ylmethyl)-2-benzofuran-1(3H)-ylidene]-1,3-dihydro-2H-indol-2-one

To a solution of oxindole (100 mg, 0.751 mmol) in dimethoxyethane (2.0 ml) at room temperature was added 1.0M LiHMDS/THF (2.25 ml). After the mixture was stirred for 10 minutes at room temperature, the solid 3-(1H-tetrazol-5-ylmethyl)-2-benzofuran-1(3H)-one (146 mg, 0.676 mmol) was added in one portion and the reaction was rapidly stirred at room temperature for 1.5 hours. The reaction was quenched into 3M HCl aqueous solution (60 ml) to give a yellow precipitate. The aqueous layer was decanted away and the solid was rinsed twice with water.

The aqueous layers were combined and extracted with EtOAc. The EtOAc portion was then extracted with saturated NaHCO₃. The aqueous portion was acidified with 3M HCl aqueous solution, and then extracted with EtOAc. The combined organic layers were washed with brine, dried with anhydrous Na₂SO₄, and rotary evaporated to provide yellow solid film A.

The above yellow precipitate was then dissolved in EtOAc by warming, washed with water, brine, dried with anhydrous Na₂SO₄, and rotary evaporated to give a yellow solid. The yellow solid was heated with MeOH and then filtered to remove insoluble solid, and give filtrate A.

The filtrate A was combined with the yellow solid film A, and was then recrystallized from dioxane to obtain a pure solid wet with dioxane. The pure solid was dissolved in 30% MeOH in CHCl₃ and then rotary evaporated to give (3E)-3-[3-(1H-tetrazol-5-ylmethyl)-2-benzofuran-1 (3H)-ylidene]-1,3-dihydro-2H-indol-2-one as a yellow solid (22 mg, 10%).

¹H NMR (300 MHz, DMSO-D6) δ ppm 3.45 (dd, J=15.54, 8.50 Hz, 1H) 3.93 (dd, J=15.54, 4.10 Hz, 1H) 6.39 (dd, J=8.21, 4.10 Hz, 1H) 6.85 (m, 2H) 7.10 (m, 1H) 7.52 (d, J=7.62 Hz, 1H) 7.61 (m, 1H) 7.70 (m, 2H) 9.60 (d, J=7.92 Hz, 1H) 10.44 (s, 1H).

### Example 128

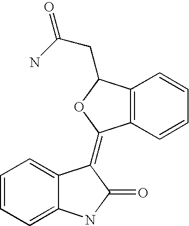

### 2-[3-(2-Oxo-1,2-dihydro-indol-3-ylidene)-1,3-dihydro-isobenzofuran-1-yl]-acetamide

To a solution of [3-(2-oxo-1,2-dihydro-indol-3-ylidene)-1,3-dihydro-isobenzofuran-1-yl]-acetic acid (100 mg, 0.325 mmol) and triethylamine (58.9 μl, 0.423 mmol) in THF (3.0 ml) was added chloroethylformate (35.7 μl, 0.374 mmol). The mixture was stirred at room temperature for 25 minutes, and then concentrated NH₄OH (3.0 ml) was added. After stirring for 8 minutes at room temperature, the reaction was partitioned between 4% HCl aqueous solution, and EtOAc. The organic layer was then washed with saturated NaHCO₃ aqueous solution, water, dilute HCl aqueous solution, brine, dried with anhydrous Na₂SO₄ and rotary evaporated to yield a solid. The solid was recrystallized from CHCl₃/MeOH to give 2-[3-(2-oxo-1,2-dihydro-indol-3-ylidene)-1,3-dihydro-isobenzofuran-1-yl]-acetamide as a yellow-gold solid (34.4 mg, 35%).

¹H NMR (500 MHz, DMSO-D6) δ ppm 2.57 (dd, J=15.13, 9.28 Hz, 1H) 2.98 (dd, J=14.89, 4.15 Hz, 1H) 6.25 (dd, J=9.28, 3.91 Hz, 1H) 6.83 (d, J=7.81 Hz, 1H) 6.93 (m, 1H) 7.12 (td, J=7.69, 1.22 Hz, 1H) 7.18 (s, 1H) 7.52 (s, 1H) 7.59 (m, 1H) 7.67 (m, 2H) 7.80 (d, J=7.32 Hz, 1H) 9.63 (d, J=8.30 Hz, 1H) 10.43 (s, 1H).

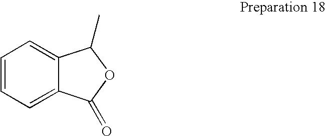

### Preparation of 3-methyl-2-benzofuran-1(3H)-one

To a solution of 2-bromobenzoic acid (0.50 g, 2.49 mmol) in THF (12.0 ml) cooled at −78° C. was added 2.5M n-BuLi in hexane (2.0 ml). After stirring for 25 minutes, a solution of acetaldehyde (0.142 g, 3.23 mmol) in THF (0.3 ml) was added. The reaction was allowed to warm to room temperature after 8 minutes at −78° C., and then quenched into 10% HCl aqueous solution (30 ml). The acidic solution was rapidly stirred for 1.5 hours, and then extracted with EtOAc. The combined organic layers were washed with saturated NaHCO₃ aqueous solution, brine, dried with anhydrous Na₂SO₄, and rotary evaporated to an oily residue. Gradient chromatography of the oil residue through a silica gel column with 20% to 30% EtOAc in hexane afforded 3-methyl-2-benzofuran-1(3H)-one as a clear oil (130 mg, 35%).

¹H NMR (300 MHz, CDCl₃) δ ppm 1.64 (d, J=6.74 Hz, 3H) 5.57 (q, J=6.74 Hz, 1H) 7.45 (dd, J=7.62, 0.88 Hz, 1H) 7.53 (t, J=7.48 Hz, 1H) 7.69 (td, J=7.55, 1.03 Hz, 1H) 7.90 (d, J=7.62 Hz, 1H).

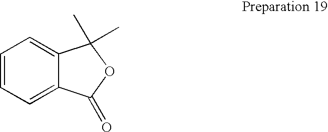

### Preparation of 3,3-dimethyl-2-benzofuran-1(3H)-one

To a solution of 2-bromobenzoic acid (500 mg, 2.49 mmol) in THF (12.0 ml) cooled at −78° C. was added 2.5M n-BuLi/Hexane (2.0 ml). After stirring for 18 minutes, acetone (0.91 ml, 12.4 mmol) was added. The reaction was allowed to warm to room temperature after 5 minutes at −78° C., and then quenched into 10% HCl aqueous solution (50 ml). The acidic solution was rapidly stirred for 1 hour, and then extracted with EtOAc. The combined organic layers were washed with saturated NaHCO₃, brine, dried with anhydrous Na₂SO₄, and rotary evaporated to give a mixture. Gradient chromatography of the mixture through silica gel column eluted with 20% to 30% EtOAc in hexane afforded 3,3-dimethyl-2-benzofuran-1(3H)-one as a white solid (161 mg, 40%).

¹H NMR (300 MHz, CDCl₃) δ ppm 1.67 (s, 6H) 7.41 (d, J=7.62 Hz, 1H) 7.51 (td, J=7.48, 0.88 Hz, 1H) 7.67 (td, J=7.55, 1.03 Hz, 1H) 7.87 (d, J=7.62 Hz, 1H).

### Example 129

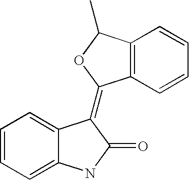

### (3E)-3-(3-methyl-2-benzofuran-1(3H)-ylidene)-1,3-dihydro-2H-indol-2-one

To a solution of oxindole (119 mg, 0.891 mmol) in THF (2.0 ml) cooled at 0° C. was added 1.0M LiHMDS/THF (1.78 ml). The mixture was stirred for 4 minutes at 0° C., then allowed to warm to room temperature for an additional 6 minutes. A solution of 3-methyl-2-benzofuran-1(3H)-one (119 mg, 0.802 mmol) in THF (0.5 ml) was added, and the reaction was rapidly stirred for 1.5 hours. The reaction was quenched into 10% HCl aqueous solution (30 ml). The resulting mixture was stirred 5 minutes, and then extracted into EtOAc. The combined organic layers were washed with water, brine, dried with anhydrous Na₂SO₄, and rotary evaporated to a yellow oil. Upon standing at room temperature overnight a yellow crystalline solid formed from the yellow oil. The remaining oil was removed by pipet and the solid was recrystallized from EtOAc to produce (3E)-3-(3-methyl-2-benzofuran-1(3H)-ylidene)-1,3-dihydro-2H-indol-2-one as a yellow solid (32 mg, 15%).

¹H NMR (300 MHz, CDCl₃) δ ppm 1.73 (d, J=6.74 Hz, 3H) 5.92 (q, J=6.45 Hz, 1H) 6.88 (m, 1H) 7.06 (td, J=7.62, 1.17 Hz, 1H) 7.16 (td, J=7.55, 1.32 Hz, 1H) 7.39 (m, 1H) 7.56 (m, 2H) 7.85 (s, 1H) 8.00 (dt, J=7.62, 0.59 Hz, 1H) 9.72 (m, 1H).

### Example 130

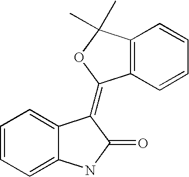

### (3E)-3-(3,3-dimethyl-2-benzofuran-1(3H)-ylidene)-1,3-dihydro-2H-indol-2-one

To a solution of oxindole (127 mg, 0.956 mmol) in THF (2.0 ml) cooled at 0° C. was added 1.0M LiHMDS in THF (1.91 ml). The mixture was stirred for 4 minutes at 0° C., then allowed to warm to room temperature for an additional 6 minutes. The solid 3,3-dimethyl-2-benzofuran-1(3H)-one (140 mg, 0.86 mmol) was added in one portion and the reaction rapidly stirred for 1.5 hours. The reaction was quenched into 30 ml 10% HCl aqueous solution. The resulting mixture was stirred 5 minutes, and then extracted into EtOAc. The combined organic layers were washed with water, brine, dried with anhydrous Na₂SO₄, and rotary evaporated to a yellow oil. The yellow oil was chromatographed through silica gel column (20% to 30% EtOAc/hexane gradient) to give (3E)-3-(3,3-dimethyl-2-benzofuran-1(3H)-ylidene)-1,3-dihydro-2H-indol-2-one as a yellow solid (99 mg, 41%).

¹H NMR (300 MHz, CDCl₃) δ ppm 1.74 (s, 6H) 6.88 (d, J=7.92 Hz, 1H) 7.06 (m, 1H) 7.16 (m, 1H) 7.33 (m, 1H) 7.54 (m, 2H) 7.87 (s, 1H) 8.00 (dd, J=7.62, 0.59 Hz, 1H) 9.70 (m, 1H).

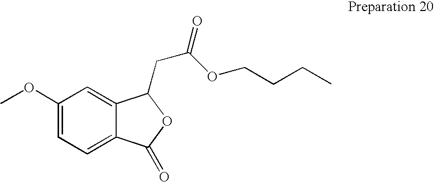

### Preparation of Butyl(6-methoxy-3-oxo-1,3-dihydro-2-benzofuran-1-yl)acetate

A mixture of 4-methoxybenzoic acid (1.52 g, 10.0 mmol), palladium (II) acetate (0.56 g, 2.5 mmol), copper (II) acetate monohydrate (0.499 g, 2.5 mmol) and 4A molecular sieves (1.75 g) in DMF (40 ml) was stirred at room temperature for 5 minutes. Butyl acrylate was add (2.57 g, 20.0 mmol), and the resulting mixture was heated at 120° C. for 25 hours and then at 140° C. for 9 hours. Upon cooling the mixture was filtered and partitioned between dilute HCl aqueous solution and EtOAc. The organic layer was washed with saturated NaHCO₃ aqueous solution, water, brine, then dried with anhydrous Na₂SO₄ and rotary evaporated to a brown oil. The oil was triturated with hexane and the remaining oil was chromatographed through silica gel column (20% to 30% EtOAc/Hexane gradient) to give the desired product contaminated with 4-methoxybenzoic acid. The mixture was dissolved in EtOAc and washed with saturated NaHCO₃ aqueous solution, brine, dried with anhydrous Na₂SO₄. Removal of the solvent yielded butyl(6-methoxy-3-oxo-1,3-dihydro-2-benzofuran-1-yl)acetate as a light yellow oil (0.26 g, 9%).

¹H NMR (300 MHz, CHLOROFORM-D) δ ppm 0.94 (t, J=7.33 Hz, 3H) 1.37 (m, 2H) 1.63 (m, 2H) 2.84 (dd, J=16.56, 6.30 Hz, 1H) 2.94 (m, 1H) 3.90 (s, 3H) 4.17 (t, J=6.60 Hz, 2H) 5.80 (t, J=6.60 Hz, 1H) 6.94 (m, 1H) 7.05 (dd, J=8.50, 2.35 Hz, 1H) 7.81 (d, J=8.50 Hz, 1H).

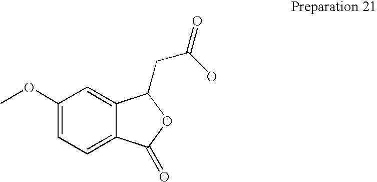

### Preparation of 6-methoxy-3-oxo-1,3-dihydro-2-benzofuran-1-yl)acetic acid

A mixture of butyl(6-methoxy-3-oxo-1,3-dihydro-2-benzofuran-1-yl)acetate (251 mg, 0.902 mmol) in 6M HCl/H₂O (10.0 ml) was heated at 80° C. for 2.5 hours. The mixture was diluted with water and extracted with EtOAc. The combined organic layers were washed with brine, dried with anhydrous Na₂SO₄, and rotary evaporated to give a pale yellow solid. The pale yellow solid was triturated at room temperature with 10% EtOAc/Hexane and then only hexane to afford (6-methoxy-3-oxo-1,3-dihydro-2-benzofuran-1-yl)acetic acid as a white solid (150.2 mg, 75%).

¹H NMR (300 MHz, DMSO-D6) δ ppm 2.66 (dd, J=16.71, 8.50 Hz, 1H) 3.18 (dd, J=16.71, 3.81 Hz, 1H) 3.87 (s, 3H) 5.78 (dd, J=8.50, 3.81 Hz, 1H) 7.12 (dd, J=8.50, 2.05 Hz, 1H) 7.28 (d, J=1.47 Hz, 1H) 7.73 (d, J=8.50 Hz, 1H) 12.59 (br s, 1H).

### Example 131

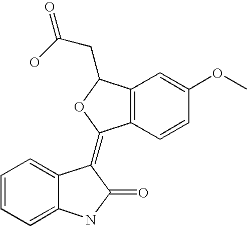

### [(3E)-6-methoxy-3-(2-oxo-1,2-dihydro-3H-indol-3-ylidene)-1,3-dihydro-2-benzofuran-1-yl]acetic acid

To a solution of oxindole (50 mg, 0.375 mmol) in dimethoxyethane (1.0 ml) at room temperature was added 1.0M LiHMDS in THF (1.13 ml), and the mixture was stirred for 10 minutes at room temperature. The solid (6-methoxy-3-oxo-1,3-dihydro-2-benzofuran-1-yl)acetic acid (75 mg, 0.338 mmol) was added in one portion and the reaction rapidly stirred at room temperature for 3 hours (an additional dimethoxyethane (0.75 ml) was added to thin the mixture in order to stir). The reaction was quenched into 10% HCl aqueous solution (50 ml). The resulting mixture was stirred 10 minutes, then extracted into EtOAc. The combined organic layers were then extracted with saturated NaHCO₃ aqueous solution. The aqueous layer was acidified with 10% HCl aqueous solution, extracted with EtOAc. The combined organic layers were washed with brine, dried with anhydrous Na₂SO₄, and rotary evaporated to give a yellow solid. The yellow solid was recrystallized from CHCl₃/MeOH to give a solid mixture (27 mg). Purification of the mixture by silica gel column chromatography, eluted with 4% MeOH/CHCl₃ to afford [(3E)-6-methoxy-3-(2-oxo-1,2-dihydro-3H-indol-3-ylidene)-1,3-dihydro-2-benzofuran-1-yl]acetic acid as a yellow solid (6.1 mg, 5%).

¹H NMR (300 MHz, ACETONE-D6) δ ppm 2.87 (dd, J=16.71, 9.09 Hz, 1H) 3.28 (dd, J=16.71, 4.10 Hz, 1H) 3.94 (s, 3H) 6.22 (dd, J=8.65, 3.96 Hz, 1H) 6.91 (m, 2H) 7.09 (m, 2H) 7.26 (d, J=1.47 Hz, 1H) 7.89 (d, J=7.92 Hz, 1H) 9.32 (s, 1H) 9.68 (d, J=8.79 Hz, 1H).

### Example 132

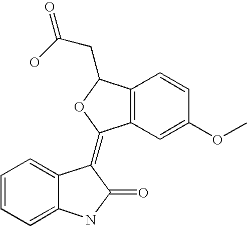

### [(3E)-5-methoxy-3-(2-oxo-1,2-dihydro-3H-indol-3-ylidene)-1,3-dihydro-2-benzofuran-1-yl]acetic acid

To a solution of oxindole (133 mg, 1.0 mmol) in dimethoxyethane (3.0 ml) cooled to 0° C. was added 1.0M LiHMDS/THF (3.0 ml). The mixture was stirred for 8 minutes at 0° C., and then the ice bath was removed. The solid (5-methoxy-3-oxo-1,3-dihydro-isobenzofuran-1-yl)-acetic acid (200 mg, 0.90 mmol) was added in one portion and the reaction mixture was rapidly stirred at room temperature for 3.5 hours. The reaction was quenched into 10% HCl aqueous solution (30 ml). After stirred for 10 minutes, the resulting mixture was extracted with EtOAc, and then the combined organic layers were then extracted with saturated NaHCO₃. The aqueous layer was acidified with 10% HCl aqueous solution, and extracted with EtOAc. The combined organic layers were washed with brine, dried with anhydrous Na₂SO₄, and rotary evaporated to produce a yellow solid. The yellow solid was recrystallized from EtOAc to give a solid (94 mg), which was recrystallized from MeOH to afford [(3E)-5-methoxy-3-(2-oxo-1,2-dihydro-3H-indol-3-ylidene)-1,3-dihydro-2-benzofuran-1-yl]acetic acid as a yellow fluffy solid (48 mg, 16%).

¹H NMR (300 MHz, d₆-DMSO) δ ppm 2.68 (dd, J=16.42, 9.09 Hz, 1H) 3.24 (dd, J=16.42, 3.81 Hz, 1H) 3.86 (s, 3H) 6.17 (dd, J=9.09, 3.81 Hz, 1H) 6.84 (d, J=7.62 Hz, 1H) 6.93 (t, J=7.62 Hz, 1H) 7.12 (t, J=7.48 Hz, 1H) 7.26 (dd, J=8.50, 2.35 Hz, 1H) 7.59 (d, J=8.50 Hz, 1H) 7.79 (d, J=7.62 Hz, 1H) 9.33 (d, J=2.35 Hz, 1H) 10.40 (s, 1H) 12.66 (s, 1H).

### Example 133

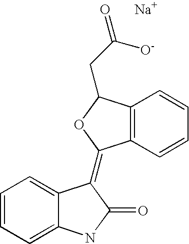

### Sodium [(3E)-3-(2-oxo-1,2-dihydro-3H-indol-3-ylidene)-1,3-dihydro-2-benzofuran-1-yl]acetate

To a slurry of [3-(2-oxo-1,2-dihydro-indol-3-ylidene)-1,3-dihydro-isobenzofuran-1-yl]-acetic acid (430 mg, 1.40 mmol) in MeOH (100.0 ml) at room temperature was added 0.5M NaOMe/MeOH (2.80 ml). The solution was rapidly stirred for 15 minutes and then rotary evaporated (combined 116 mg of product obtained from previously run reaction). The solid was chased with MeOH (20 ml), and then with EtOAc (2×15 ml) to give sodium [(3E)-3-(2-oxo-1,2-dihydro-3H-indol-3-ylidene)-1,3-dihydro-2-benzofuran-1-yl]acetate as a yellow solid (576 mg, 100%).

¹H NMR (300 MHz, DMSO-D6) δ ppm 2.52 (obsc m, 2H) 6.23 (t, J=6.89 Hz, 1H) 6.82 (d, J=7.62 Hz, 1H) 6.93 (t, J=7.62 Hz, 1H) 7.10 (t, J=7.77 Hz, 1H) 7.53 (t, J=7.48 Hz, 1H) 7.61 (t, J=7.18 Hz, 1H) 7.73 (d, J=7.33 Hz, 1H) 7.86 (d, J=7.33 Hz, 1H) 9.63 (d, J=7.92 Hz, 1H) 10.43 (s, 1H).

### Example 134

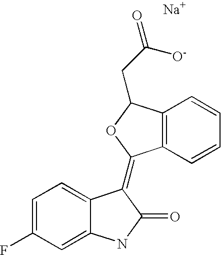

### Sodium [(3E)-3-(6-fluoro-2-oxo-1,2-dihydro-3H-indol-3-ylidene)-1,3-dihydro-2-benzofuran-1-yl]acetate

To a slurry of [3-(6-fluoro-2-oxo-1,2-dihydro-indol-3-ylidene)-1,3-dihydro-isobenzofuran-1-yl]-acetic acid (521 mg, 1.60 mmol) in MeOH (100.0 ml) at room temperature was added 0.5M NaOMe/MeOH (3.20 ml). The solution was rapidly stirred for 15 minutes and then rotary evaporated (combined 116 mg of product obtained from previously run reaction). The solid was chased with MeOH (20 ml) and then EtOAc (15 ml) to give sodium [(3E)-3-(6-fluoro-2-oxo-1,2-dihydro-3H-indol-3-ylidene)-1,3-dihydro-2-benzofuran-1-yl]acetate as a yellow solid (557 mg, 100%).

¹H NMR (300 MHz, DMSO-D6) δ ppm 2.52 (obsc m, 2H) 6.23 (t, J=6.89 Hz, 1H) 6.70 (m, 2H) 7.54 (t, J=7.33 Hz, 1H) 7.62 (t, J=7.04 Hz, 1H) 7.74 (d, J=7.33 Hz, 1H) 7.83 (dd, J=8.21, 5.86 Hz, 1H) 9.57 (d, J=7.92 Hz, 1H) 10.68 (s, 1H).

The following Example 135 was prepared using the experiment procedure described in Example 136, but with the appropriate reagent, reaction conditions and reactant substitutions that will be readily realized by those of ordinary skill in this art without the exercise of undue experimentation.

### Example 135

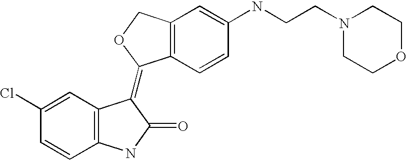

### 5-Chloro-3-[5-(2-morpholin-4-yl-ethylamino)-3H-isobenzofuran-1-ylidene]-1,3-dihydro-indol-2-one

¹H NMR (500 MHz, DMSO-d₆) δ ppm 2.43 (br s, 4H) 2.53 (t, J=6.59 Hz, 2H) 3.28 (q, J=5.86 Hz, 2H) 3.59 (t, J=4.39 Hz, 4H) 5.67 (s, 2H) 6.73 (s, 1H) 6.76 (m, 2H) 6.81 (t, J=5.37 Hz, 1H) 7.03 (dd, J=8.05, 2.20 Hz, 1H) 7.70 (d, J=2.44 Hz, 1H) 9.33 (d, J=9.28 Hz, 1H) 10.34 (s, 1H).

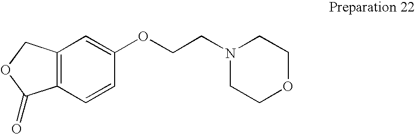

### Preparation of 5-(2-Morpholin-4-yl-ethoxy)-3H-isobenzofuran-1-one

A mixture of 5-hydroxyphthalide (1.0 g, 6.66 mmol), 4-(2-chloroethyl)-morpholine hydrochloride (1.49 g, 8.01 mmol), potassium carbonate (2.3 g, 16.6 mmol) and sodium iodide (1.0 g, 6.67 mmol) in ethanol (40 ml) was stirred at reflux under nitrogen for 18 hours. The mixture was cooled to room temperature, and filtered through celite. The filtrate solution was evaporated to dryness. The residue was partitioned between EtOAc (75 ml) and 2M HCl solution (50 ml). The organic layer was extracted with 2M HCl (2×30 ml). The aqueous layers were combined, basified with NaOH aqueous solution, and extracted with CHCl₃ (3×50 ml). The organic layers were combined, dried over anhydrous Na₂SO₄, and evaporated to a light brown solid, which was triturated with CHCl₃/Hexanes to give 5-(2-morpholin-4-yl-ethoxy)-3H-isobenzofuran-1-one as white crystals (1.43 g, 82%).

¹H NMR (500 MHz, CDCl₃) δ ppm 2.74 (br s, 4H) 2.98 (br s, 2H) 3.83 (br s, 4H) 4.31 (br s, 2H) 5.25 (s, 2H) 6.95 (s, 1H) 7.05 (dd, J=8.54, 2.20 Hz, 1H) 7.83 (d, J=8.30 Hz, 1H).

### Example 136

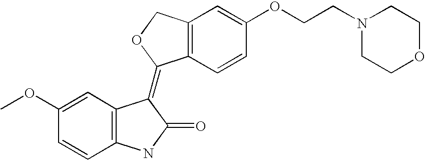

### 5-Methoxy-3-[5-(2-morpholin-4-yl-ethoxy)-3H-isobenzofuran-1-ylidene]-1,3-dihydro-indol-2-one

To a stirred solution of 5-methoxyoxindole (197 mg, 1.21 mmol) in anhydrous THF (10 ml) under nitrogen was added 1.0M LiHMDS/THF solution (2.8 ml, 2.8 mmol). The mixture was stirred at room temperature for 10 minutes, and 5-(2-morpholin-4-yl-ethoxy)-3H-isobenzofuran-1-one (200 mg, 0.76 mmol) was added. After stirred at room temperature for 3.0 hours, the mixture was poured into a mixture of THF (5 ml) and 2M HCl aqueous solution (10 ml), and heated at 60° C. for 40 minutes. The mixture was cooled to room temperature, basified with 5M NaOH aqueous solution, and then poured into water (150 ml). The resulting solid was separated, rinsed with water, and dried to give 5-methoxy-3-[5-(2-morpholin-4-yl-ethoxy)-3H-isobenzofuran-1-ylidene]-1,3-dihydro-indol-2-one as a yellow solid (275 mg, 89%).

¹H NMR (500 MHz, DMSO-d₆) δ ppm 2.43 (br s, 4H) 2.68 (t, J=5.61 Hz, 2H) 3.53 (t, J=4.88 Hz, 4H) 3.68 (s, 3H) 4.16 (t, J=5.61 Hz, 2H) 5.69 (s, 2H) 6.63 (m, 2H) 7.07 (dd, J=9.03, 2.20 Hz, 1H) 7.17 (s, 1H) 7.36 (d, J=2.44 Hz, 1H) 9.52 (d, J=9.27 Hz, 1H) 10.12 (s, 1H).

The following Example 137 through 139 were prepared using the experiment procedure described in Example 136, but with the appropriate reagent, reaction conditions and reactant substitutions that will be readily realized by those of ordinary skill in this art without the exercise of undue experimentation

### Example 137

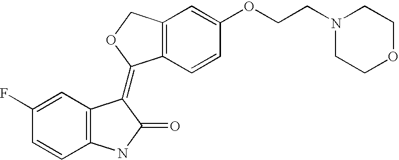

### 5-Fluoro-3-[5-(2-morpholin-4-yl-ethoxy)-3H-isobenzofuran-1-ylidene]-1,3-dihydro-indol-2-one

¹H NMR (500 MHz, DMSO-d₆) δ ppm 2.44 (br s, 4H) 2.68 (t, J=5.61 Hz, 2H) 3.53 (t, J=4.39 Hz, 4H) 4.17 (t, J=5.61 Hz, 2H) 5.72 (s, 2H) 6.72 (dd, J=8.54, 4.64 Hz, 1H) 6.84 (m, 1H) 7.09 (dd, J=9.28, 2.44 Hz, 1H) 7.20 (d, J=1.95 Hz, 1H) 7.49 (dd, J=9.52, 2.68 Hz, 1H) 9.50 (d, J=8.79 Hz, 1H) 10.33 (s, 1H).

### Example 138

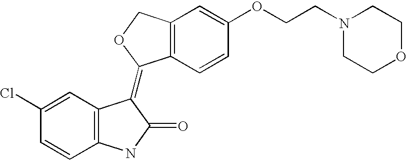

### 5-Chloro-3-[5-(2-morpholin-4-yl-ethoxy)-3H-isobenzofuran-1-ylidene]-1,3-dihydro-indol-2-one

¹H NMR (500 MHz, DMSO-d₆) δ ppm 2.44 (br s, 4H) 2.68 (t, J=5.37 Hz, 2H) 3.53 (t, J=4.39 Hz, 4H) 4.17 (t, J=5.37 Hz, 2H) 5.73 (s, 2H) 6.75 (d, J=8.30 Hz, 1H) 7.05 (dd, J=8.30, 1.95 Hz, 1H) 7.10 (dd, J=8.79, 1.95 Hz, 1H) 7.21 (s, 1H) 7.71 (s, 1H) 9.49 (d, J=8.79 Hz, 1H) 10.45 (s, 1H)

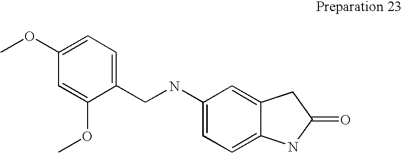

### Preparation of 5-(2,4-Dimethoxy-benzylamino)-1,3-dihydro-indol-2-one

A mixture of 5-aminooxindole (1.0 g, 6.75 mmol), 3,5-dimethoxybenzaldehyde (1.35 g, 8.12 mmol), sodium triacetoxyborohydride (4.3 g, 20.3 mmol), and AcOH (0.5 ml) in DMF (15 ml) was stirred at room temperature for 3.5 hours. The mixture was partitioned between saturated NaHCO₃ solution (50 ml) and CHCl₃ (50 ml). The aqueous layer was extracted again with CHCl₃ (2×50 ml). The organic layers were combined, washed with saturated NaHCO₃ solution (50 ml), water (2×75 ml), dried over Na₂SO₄, and poured into Et₂O (100 ml) with stirring. The resulting solid was filtered, washed with Et₂O and dried to give 5-(2,4-dimethoxy-benzylamino)-1,3-dihydro-indol-2-one as a brown solid (1.02 g, 51%).

¹H NMR (500 MHz, DMSO-d₆) δ ppm 3.32 (s, 2H) 3.72 (s, 3H) 3.79 (s, 3H) 4.06 (s, 2H) 5.52 (br s, 1H) 6.33 (dd, J=8.30, 1.95 Hz, 1H) 6.44 (dd, J=8.30, 2.44 Hz, 1H) 6.52 (m, 3H) 7.12 (d, J=8.30 Hz, 1H) 9.91 (s, 1H).

### Example 139

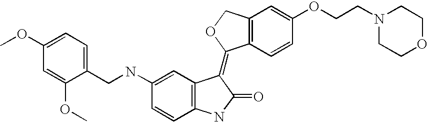

### 5-(2,4-Dimethoxy-benzylamino)-3-[5-(2-morpholin-4-yl-ethoxy)-3H-isobenzofuran-1-ylidene]-1,3-dihydro-indol-2-one

¹H NMR (500 MHz, DMSO-d₆) δ ppm 2.48 (br s, 4H) 2.73 (t, J=5.61 Hz, 2H) 3.58 (t, J=4.39 Hz, 4H) 3.72 (s, 3H) 3.83 (s, 3H) 4.11 (d, J=5.37 Hz, 2H) 4.21 (t, J=5.86 Hz, 2H) 5.47 (t, J=5.86 Hz, 1H) 5.70 (s, 2H) 6.28 (dd, J=8.30, 2.44 Hz, 1H) 6.45 (dd, J=8.54, 2.20 Hz, 1H) 6.51 (d, J=8.30 Hz, 1H) 6.55 (d, J=2.44 Hz, 1H) 7.10 (dd, J=8.79, 2.44 Hz, 1H) 7.19 (dd, J=5.13, 3.17 Hz, 2H) 7.24 (d, J=2.44 Hz, 1H) 9.58.

TABLE 7

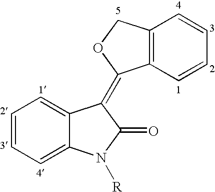

| Example | | | Molecular |
|---|---|---|---|
| Number | Chemical Structure | Chemical Name | Weight |
| 140 | 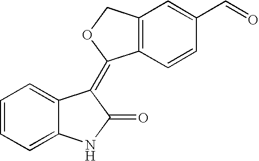 | 1-(2-Oxo-1,2-dihydro-indol-3- ylidene)-1,3-dihydro- isobenzofuran-5-carbaldehyde | 277.278 |
| 141 | 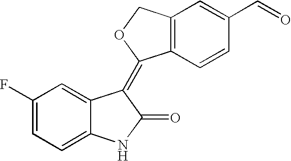 | 1-(5-Fluoro-2-oxo-1,2-dihydro- indol-3-ylidene)-1,3-dihydro- isobenzofuran-5-carbaldehyde | 295.268 |
| 142 | 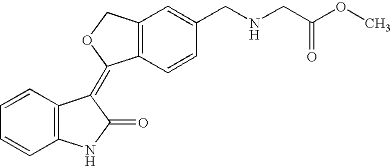 | {[1-(2-Oxo-1,2-dihydro-indol- 3-ylidene)-1,3-dihydro- isobenzofuran-5-ylmethyl]- amino}-acetic acid methyl ester | 350.372 |
| 143 | 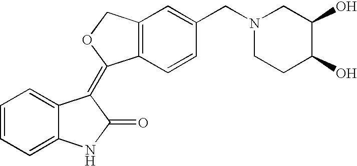 | cis-3-[5-(3,4-Dihydroxy- piperidin-1-ylmethyl)-3H- isobenzofuran-1-ylidenel-1,3- dihydro-indol-2-one | 378.426 |
| 144 | 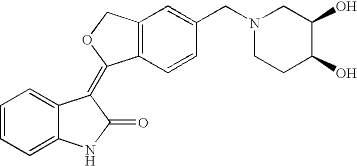 | 3-(5-Morpholin-4-ylmethyl-3H- isobenzofuran-1-ylidene)-1,3- dihydro-indol-2-one | 348.4 |
| 145 | 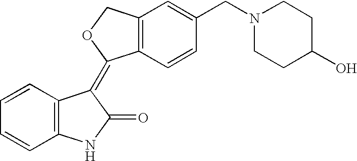 | 3-[5-(4-Hydroxy-piperidin-1- ylmethyl)-3H-isobenzofuran-1- ylidene]-1,3-dihydro-indol-2- one | 362.427 |
| 146 | 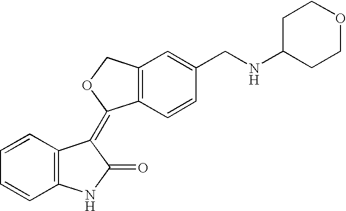 | 3-{5-[(Tetrahydro-pyran-4- ylamino)-methyl]-3H- isobenzofuran-1-ylidene}-1,3- dihydro-indol-2-one | 362.427 |
| 147 | 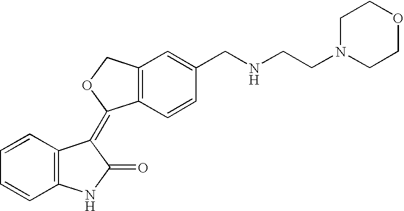 | 3-{5-[(2-Morpholin-4-yl- ethylamino)-methyl]-3H- isobenzofuran-1-ylidene}-1,3- dihydro-indol-2-one | 391.469 |
| 148 | 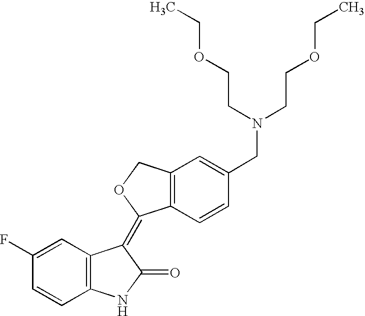 | 3-(5-{[Bis-(2-ethoxy-ethyl)- amino]-methyl}-3H- isobenzofuran-1-ylidene)-5- fluoro-1,3-dihydro-indol-2-one | 440.512 |
| 149 | 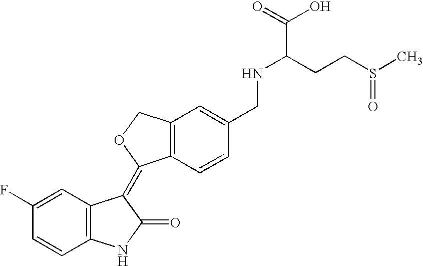 | 2-{(1-(5-Fluoro-2-oxo-1,2- dihydro-indol-3-ylidene)-1,3- dihydro-isobenzofuran-5- ylmethyl]-amino}-4- methanesulfinyl-butyric acid | 444.481 |
| 150 | 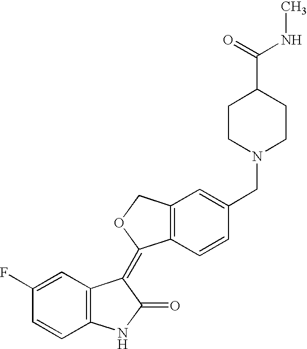 | 1-[1-(5-Fluoro-2-oxo-1,2- dihydro-indol-3-ylidene)-1,3- dihydro-isobenzofuran-5- ylmethyl]-piperidine-4- carboxylic acid methylamide | 421.47 |
| 151 | 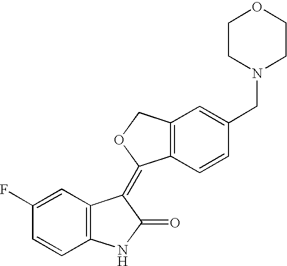 | 5-Fluoro-3-(5-morpholin-4- ylmethyl-3H-isobenzofuran-1- ylidene)-1,3-dihydro-indol-2- one | 366.39 |
| 152 | 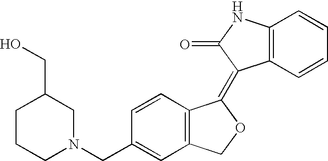 | 3-[5-(3-Hydroxymethyl- piperidin-1-ylmethyl)-3H- isobenzofuran-1-ylidene]-1,3- dihydro-indol-2-one | 376.454 |
| 153 | 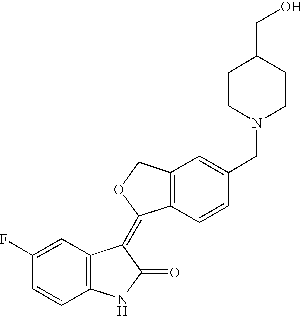 | 5-Fluoro-3-[5-(4- hydroxymethyl-piperidin-1- ylmethyl)-3H-isobenzofuran-1- ylidene]-1,3-dihydro-indol-2- one | 394.444 |
| 154 | 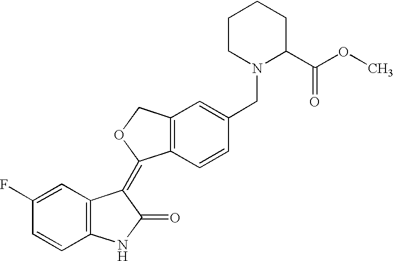 | 1-[1-(5-Fluoro-2-oxo-1,2- dihydro-indol-3-ylidene)-1,3- dihydro-isobenzofuran-5- ylmethyl]-piperidine-2- carboxylic acid methyl ester | 422.454 |
| 155 | 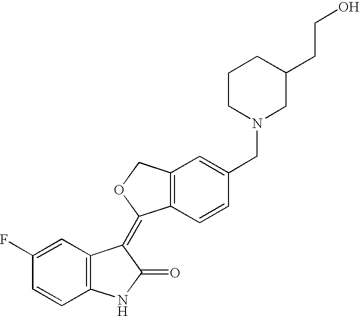 | 5-Fluoro-3-{5-[3-(2-hydroxy- ethyl)-piperidin-1-ylmethyl]- 3H-isobenzofuran-1-ylidene}- 1,3-dihydro-indol-2-one | 408.47 |
| 156 | 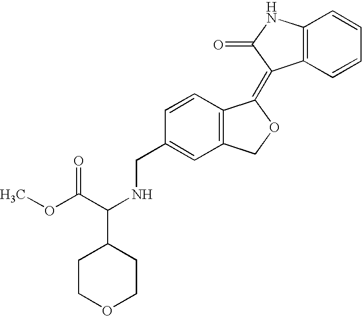 | {[1-(2-Oxo-1,2-dihydro-indol- 3-ylidene)-1,3-dihydro- isobenzofuran-5-ylmethyl]- amino}-(tetrahydro-pyran-4- yl)-acetic acid methyl ester | 434.489 |
| 157 | 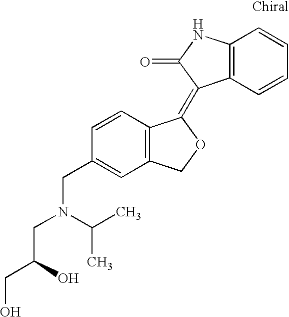 | 3-(5-{[((2S)-2,3-Dihydroxy- propyl)-isopropyl-amino]- methyl}-3H-isobenzofuran-1- ylidene)-1,3-dihydro-indol-2- one | 394.468 |
| 158 | 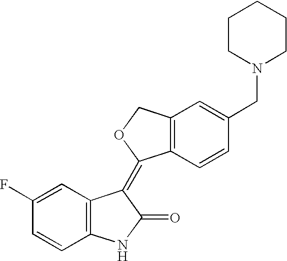 | 5-Fluoro-3-(5-piperidin-1- ylmethyl-3H-isobenzofuran-1- ylidene)-1,3-dihydro-indol-2- one | 364.418 |
| 159 | 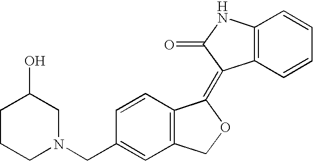 | 3-[5-(3-Hydroxy-piperidin-1- ylmethyl)-3H-isobenzofuran-1- ylidene]-1,3-dihydro-indol-2- one | 362.427 |
| 160 | 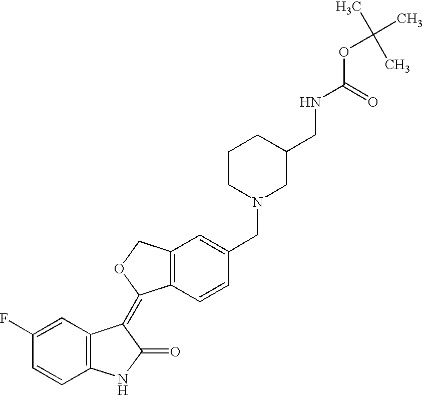 | {1-[1-(5-Fluoro-2-oxo-1,2- dihydro-indol-3-ylidene)-1,3- dihydro-isobenzofuran-5- ylmethyl]-piperidin-3- ylmethyl[carbamic acid tert- butyl ester | 493.576 |
| 161 | 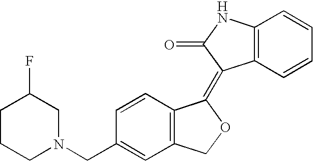 | 3-[5-(3-Fluoro-piperidin-1- ylmethyl)-3H-isobenzofuran-1- ylidene]-1,3-dihydro-indol-2- one | 364.418 |
| 162 | 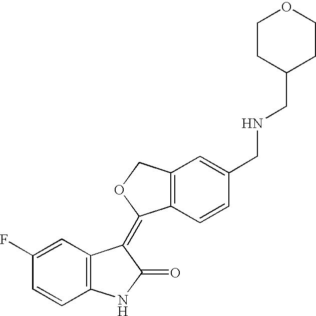 | 5-Fluoro-3-(5-{[(tetrahydro- pyran-4-ylmethyl)-amino]- methyl}-3H-isobenzofuran-1- ylidene)-1,3-dihydro-indol-2- one | 394.444 |
| 163 | 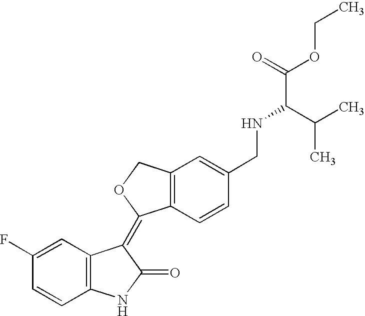 | (S)-2-{[1-(5-Fluoro-2-oxo-1,2- dihydro-indol-3-ylidene)-1,3- dihydro-isobenzofuran-5- ylmethyl]-amino}-3-methyl- butyric acid ethyl ester | 424.47 |
| 164 | 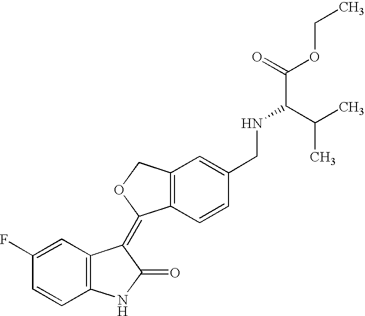 | 3-(5-{[((2R)-2,3-Dihydroxy- propyl)-isopropyl-amino]- methyl}-3H-isobenzofuran-1- ylidene)-5-fluoro-1,3-dihydro- indol-2-one | 412.458 |
| 165 | 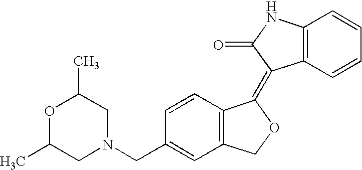 | 3-[5-(2,6-Dimethyl-morpholin- 4-ylmethyl)-3H-isobenzofuran- 1-ylidene]-1,3-dihydro-indol-2- one | 376.454 |
| 166 | 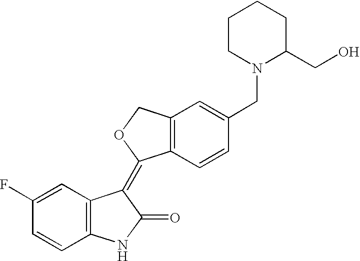 | 5-Fluoro-3-[5-(2- hydroxymethyl-piperidin-1- ylmethyl)-3H-isobenzofuran-1- ylidene]-1,3-dihydro-indol-2- one | 394.444 |
| 167 | 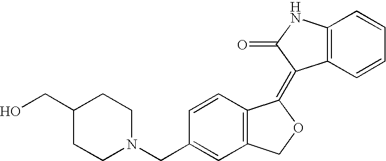 | 3-[5-(4-Hydroxymethyl- piperidin-1-ylmethyl)-3H- isobenzofuran-1-ylidene]-1,3- dihydro-indol-2-one | 376.454 |
| 168 | 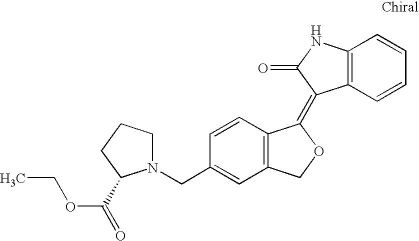 | 1-[1-(2-Oxo-1,2-dihydro-indol- 3-ylidene)-1,3-dihydro- isobenzofuran-5-ylmethyl]- pyrrolidine-(S)-2-carboxylic acid ethyl ester | 404.464 |
| 169 | 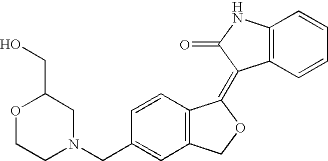 | 3-[5-(2-Hydroxymethyl- morpholin-4-ylmethyl)-3H- isobenzofuran-1-ylidene]-1,3- dihydro-indol-2-one | 378.426 |
| 170 | 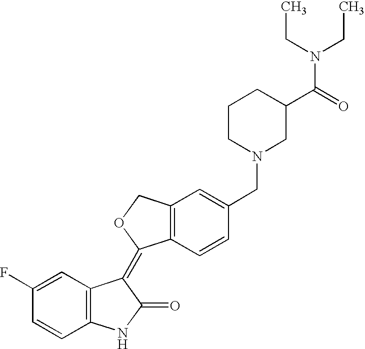 | 1-[1-(5-Fluoro-2-oxo-1,2- dihydro-indol-3-ylidene)-1,3- dihydro-isobenzofuran-5- ylmethyl]-piperidine-3- carboxylic acid diethylamide | 463.55 |
| 171 | 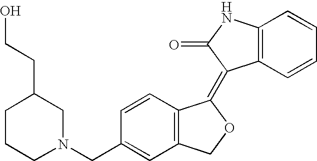 | 3-{5-[3-(2-Hydroxy-ethyl)- piperidin-1-ylmethyl]-3H- isobenzofuran-1-ylidenel-1,3- dihydro-indol-2-one | 390.48 |
| 172 | 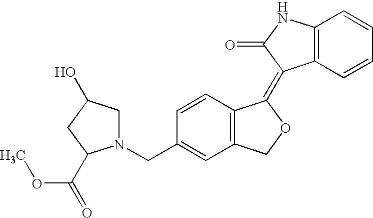 | 4-Hydroxy-1-[1-(2-oxo-1,2- dihydro-indol-3-ylidene)-1,3- dihydro-isobenzofuran-5- ylmethyl]-pyrrolidine-2- carboxylic acid methyl ester | 406.436 |
| 173 | 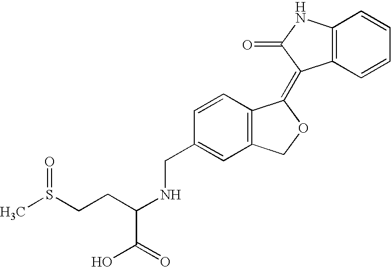 | 4-Methanesulfinyl-2-{[1-(2- oxo-1,2-dihydro-indol-3- ylidene)-1,3-dihydro- isobenzofuran-5-ylmethyl]- amino}-butyric acid | 426.493 |
| 174 | 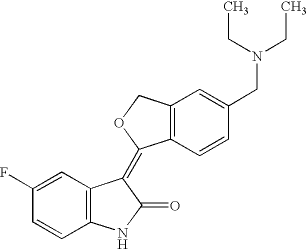 | 3-(5-Diethylaminomethyl-3H- isobenzofuran-1-ylidene)-5- fluoro-1,3-dihydro-indol-2-one | 352.407 |
| 175 | 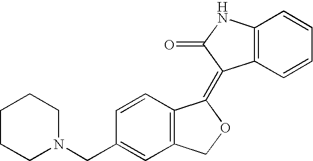 | 3-(5-Piperidin-1-ylmethyl-3H- isobenzofuran-1-ylidene)-1,3- dihydro-indol-2-one | 346.428 |
| 176 | 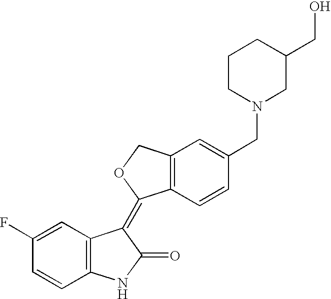 | 5-Fluoro-3-[5-(3- hydroxymethyl-piperidin-1- ylmethyl)-3H-isobenzofuran-1- -ylidene]-1,3-dihydro-indol-2- one | 394.444 |
| 177 | 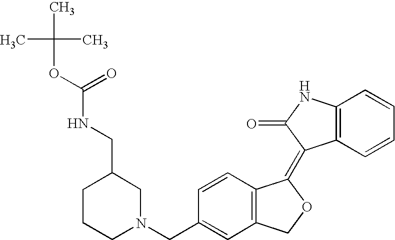 | {1-[1-(2-Oxo-1,2-dihydro- indol-3-ylidene)-1,3-dihydro- isobenzofuran-5-ylmethyl]- piperidin-3-ylmethyll-carbamic acid tert-butyl ester | 475.586 |
| 178 | 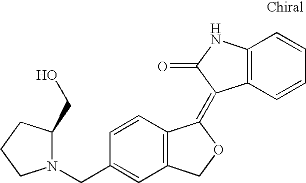 | 3-[5-((S)-2-Hydroxymethyl- pyrrolidin-1-ylmethyl)-3H- isobenzofuran-1-ylidene]-1,3- dihydro-indol-2-one | 362.427 |
| 179 | 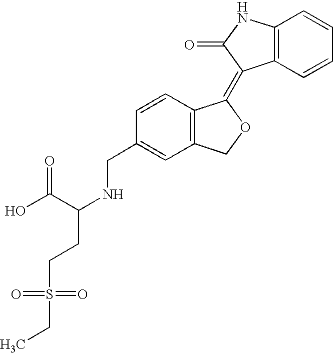 | 4-Ethanesulfonyl-2-{[1-(2-oxo- 1,2-dihydro-indol-3-ylidene)- 1,3-dihydro-isobenzofuran-5- ylmethyl]-amino}-butyric acid | 456.517 |
| 180 | 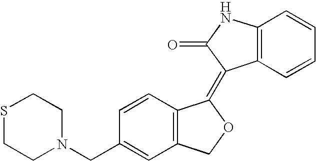 | 3-(5-Thiomorpholin-4- ylmethyl-3H-isobenzofuran-1- ylidene)-1,3-dihydro-indol-2- one | 364.467 |
| 181 | 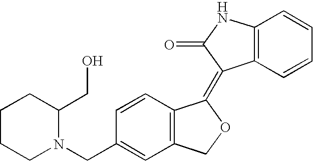 | 3-[5-(2-Hydroxymethyl- piperidin-1-ylmethyl)-3H- isobenzofuran-1-ylidene]-1,3- dihydro-indol-2-one | 376.454 |
| 182 | 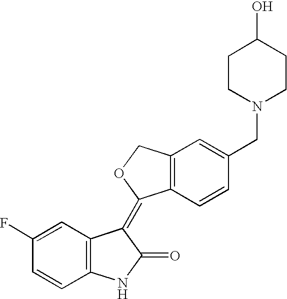 | 5-Fluoro-3-[5-(4-hydroxy- piperidin-1-ylmethyl)-3H- isobenzofuran-1-ylidene]-1,3- dihydro-indol-2-one | 380.417 |
| 183 | 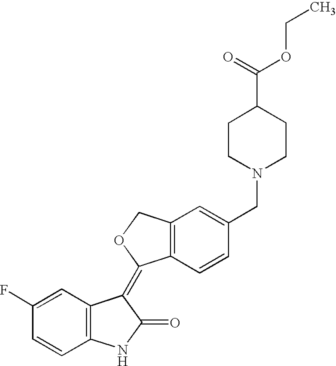 | 1-[1-(5-Fluoro-2-oxo-1,2- dihydro-indol-3-ylidene)-1,3- dihydro-isobenzofuran-5- ylmethyl]-piperidine-4- carboxylic acid ethyl ester | 436.48 |
| 184 | 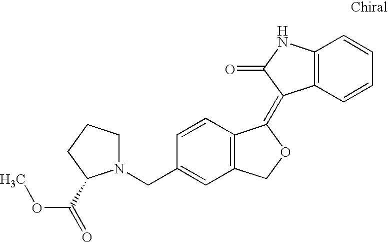 | 1-[1-(2-Oxo-1,2-dihydro-indol- 3-ylidene)-1,3-dihydro- isobenzofuran-5-ylmethyl]- pyrrolidine-(S)-2-carboxylic acid methyl ester | 390.437 |
| 185 | 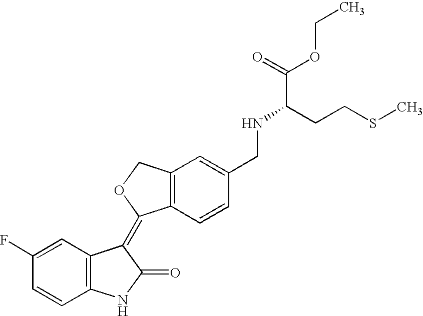 | (S)-2-{[1-(5-Fluoro-2-oxo-1,2- dihydro-indol-3-ylidene)-1,3- dihydro-isobenzofuran-5- ylmethyl]-amino}-4- methylsulfanyl-butyric acid ethyl ester | 456.535 |
| 186 | 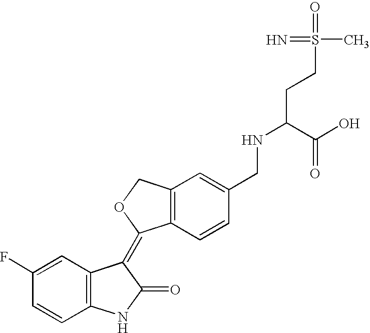 | 4-Methylsulfonimidosyl-2-{[1- (5-fluoro-2-oxo-1,2-dihydro- indol-3-ylidene)-1,3-dihydro- isobenzofuran-5-ylmethyl]- amino}-butanoic acid | 459.496 |
| 187 | 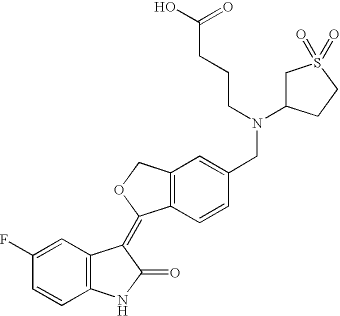 | 4-{(1,1-Dioxo-tetrahydro- 1lambda*6*-thiophen-3-yl)-[1- (5-fluoro-2-oxo-1,2-dihydro- indol-3-ylidene)-1,3-dihydro- isobenzofuran-5-ylmethyl]- amino}-butyric acid | 500.544 |
| 188 | 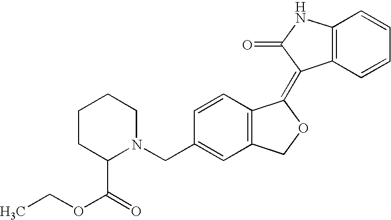 | 1-[1-(2-Oxo-1,2-dihydro-indol- 3-ylidene)-1,3-dihydro- isobenzofuran-5-ylmethyl]- piperidine-2-carboxylic acid ethyl ester | 418.49 |
| 189 | 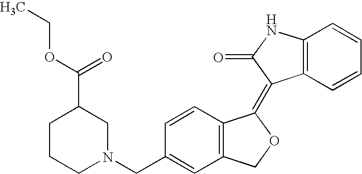 | 1-[1-(2-Oxo-1,2-dihydro-indol- 3-ylidene)-1,3-dihydro- isobenzofuran-5-ylmethyl]- piperidine-3-carboxylic acid ethyl ester | 418.49 |
| 190 | 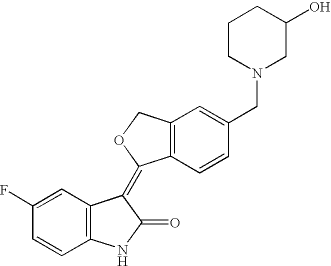 | 5-Fluoro-3-[5-(3-hydroxy- piperidin-1-ylmethyl)-3H- isobenzofuran-1-ylidene]-1,3- dihydro-indol-2-one | 380.417 |
| 191 | 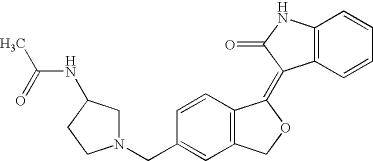 | N-{1-[1-(2-Oxo-1,2-dihydro- indol-3-ylidene)-1,3-dihydro- isobenzofuran-5-ylmethyl]- pyrrolidin-3-yl}-acetamide | 389.453 |
| 192 | 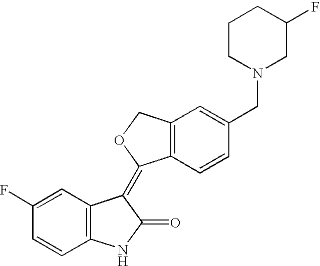 | 5-Fluoro-3-[5-(3-fluoro- piperidin-1-ylmethyl)-3H- isobenzofuran-1-ylidene]-1,3- dihydro-indol-2-one | 382.408 |
| 193 | 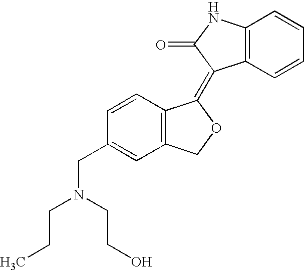 | 3-(5-{[(2-Hydroxy-ethyl)- propyl-aminol-methyl}-3H- isobenzofuran-1-ylidene)-1,3- dihydro-indol-2-one | 364.443 |
| 194 | 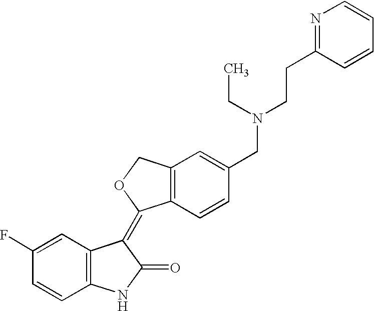 | 3-(5-{[Ethyl-(2-pyridin-2-yl- ethyl)-amino]-methyl}-3H- isobenzofuran-1-ylidene)-5- fluoro-1,3-dihydro-indol-2-one | 429.493 |
| 195 | 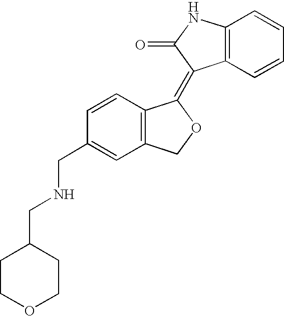 | 3-(5-{[(Tetrahydro-pyran-4- ylmethyl)-amino]-methyl}-3H- isobenzofuran-1-ylidene)-1,3- dihydro-indol-2-one | 376.454 |
| 196 | 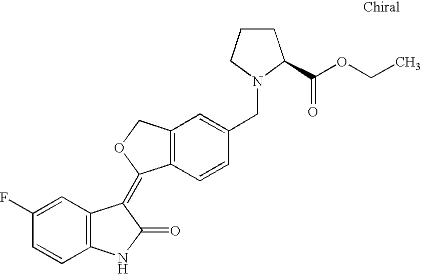 | 1-[1-(5-Fluoro-2-oxo-1,2- dihydro-indol-3-ylidene)-1,3- dihydro-isobenzofuran-5- ylmethyl]-pyrrolidine-(S)-2- carboxylic acid ethyl ester | 422.454 |
| 197 | 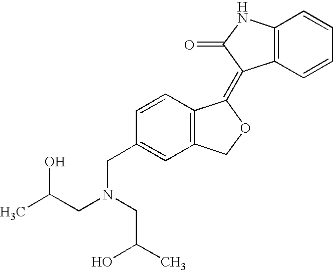 | 3-(5-{[Bis-(2-hydroxy-propyl)- amino]-methyl}-3H- isobenzofuran-1-ylidene)-1,3- dihydro-indol-2-one | 394.468 |
| 198 | 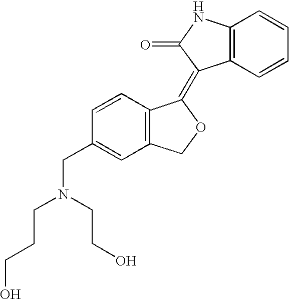 | 3-(5-{[(2-Hydroxy-ethyl)-(3- hydroxy-propyl)-amino]- methyl}-3H-isobenzofuran-1- ylidene)-1,3-dihydro-indol-2- one | 380.442 |
| 199 | 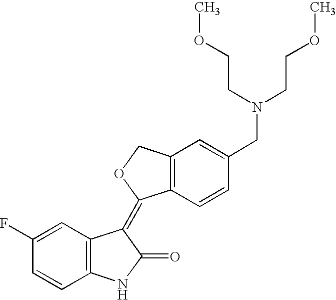 | 3-(5-{[Bis-(2-methoxy-ethyl)- amino]-methyl}-3H- isobenzofuran-1-ylidene)-5- fluoro-1,3-dihydro-indol-2-one | 412.458 |
| 200 | 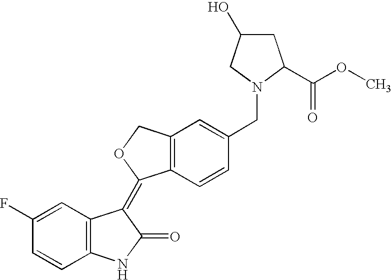 | 1-[1-(5-Fluoro-2-oxo-1,2- dihydro-indol-3-ylidene)-1,3- dihydro-isobenzofuran-5- ylmethyl]-4-hydroxy- pyrrolidine-2-carboxylic acid methyl ester | 424.426 |
| 201 | 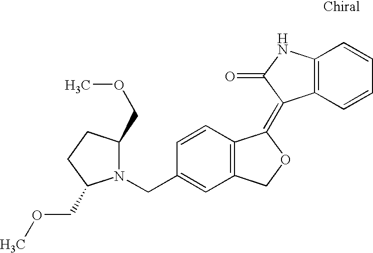 | 3-[5-((S,S)-2,5-Bis- methoxymethyl-pyrrolidin-1- ylmethyl)-3H-isobenzofuran-1- ylidene]-1,3-dihydro-indol-2- one | 420.506 |
| 202 | 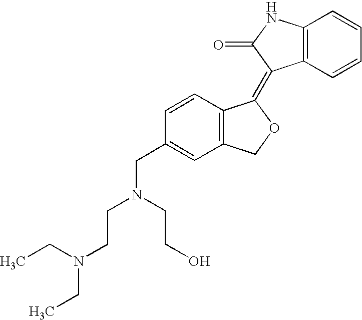 | 3-(5-{[(2-Diethylamino-ethyl) (2-hydroxy-ethyl)-amino]- methyl}-3H-isobenzofuran-1- ylidene)-1,3-dihydro-indol-2- one | 421.538 |
| 203 | 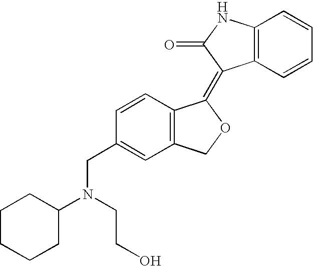 | 3-(5-{[Cyclohexyl-(2-hydroxy- ethyl)-amino]-methyl}-3H- isobenzofuran-1-ylidene)-1,3- dihydro-indol-2-one | 404.507 |
| 204 | 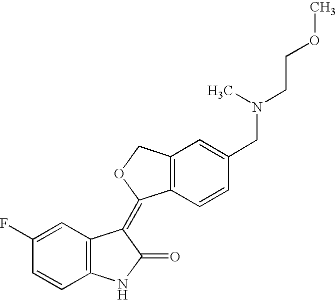 | 5-Fluoro-3-(5-{[(2-methoxy- ethyl)-methyl-amino]-methyl}- 3H-isobenzofuran-1-ylidene)- 1,3-dihydro-indol-2-one | 368.406 |
| 205 | 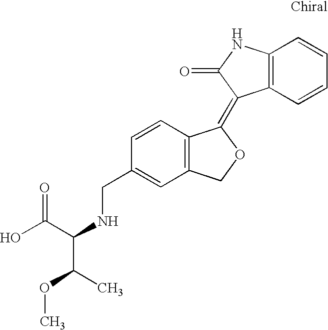 | (R)-3-Methoxy-(S)-2-{[1-(2- oxo-1,2-dihydro-indol-3- ylidene)-1,3-dihydro- isobenzofuran-5-ylmethyl]- amino}-butyric acid | 394.425 |
| 206 | 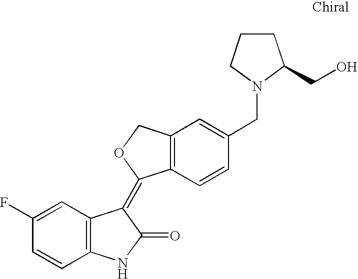 | 5-Fluoro-3-{5-[(S)-2- hydroxymethyl-pyrrolidin-1- ylmethyl}-3H-isobenzofuran-1- ylidene]-1,3-dihydro-indol-2- one | 380.417 |
| 207 | 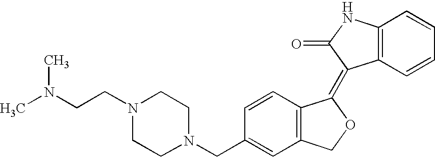 | 3-{5-[4-(2-Dimethylamino- ethyl)-piperazin-1-ylmethyl]- 3H-isobenzofuran-1-ylidene}1,3-dihydro-indol-2-one | 418.538 |
| 208 | 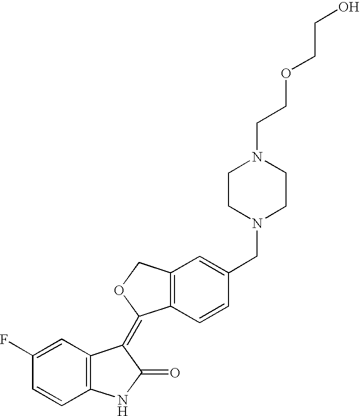 | 5-Fluoro-3-(5-{4-[2-(2- hydroxy-ethoxy)-ethyl]- piperazin-1-ylmethyl}-3H- isobenzofuran-1-ylidene)-1,3- dihydro-indol-2-one | 453.511 |
| 209 | 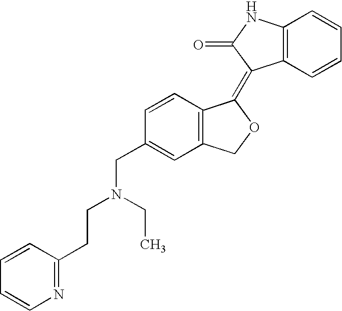 | 3-(5-{[Ethyl-(2-pyridin-2-yl- ethyl)-amino]-methyl}-3H- isobenzofuran-1-ylidene)-1,3- dihydro-indol-2-one | 411.503 |
| 210 | 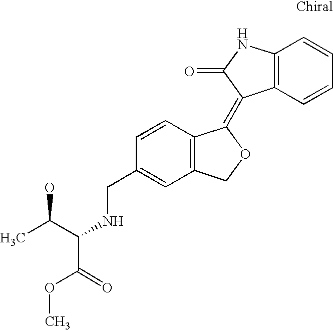 | (R)-3-Hydroxy-(S)-2-{[1-(2- oxo-1,2-dihydro-indol-3- ylidene)-1,3-dihydro- isobenzofuran-5-ylmethyl]- amino}-butyric acid methyl ester | 394.425 |
| 211 | 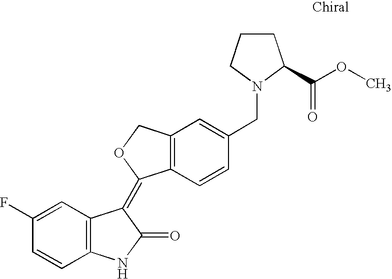 | 1-[1-(5-Fluoro-2-oxo-1,2- dihydro-indol-3-ylidene)-1,3- dihydro-isobenzofuran-5- ylmethyl]-pyrrolidine-(S)-2- carboxylic acid methyl ester | 408.427 |
| 212 | 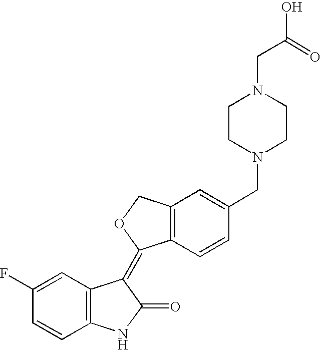 | {4-[1-(5-Fluoro-2-oxo-1,2- dihydro-indol-3-ylidene)-1,3- dihydro-isobenzofuran-5- ylmethyl]-piperazin-1-yl}-acetic acid | 423.442 |
| 213 | 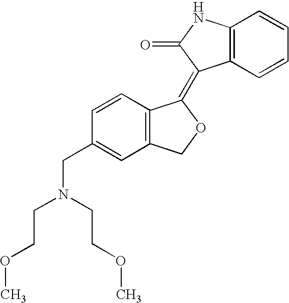 | 3-(5-{[Bis-(2-methoxy-ethyl)- amino]-methyl}-3H- isobenzofuran-1-ylidene)-1,3- dihydro-indol-2-one | 394.468 |
| 214 | 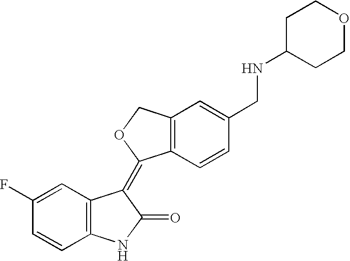 | 5-Fluoro-3-{5-[(tetrahydro- pyran-4-ylamino)-methyl]-3H- isobenzofuran-1-ylidene}-1,3- dihydro-indol-2-one | 380.417 |
| 215 | 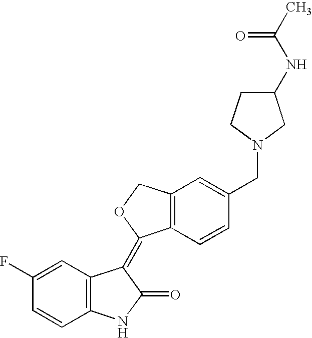 | N-{1-[1-(5-Fluoro-2-oxo-1,2- dihydro-indol-3-ylidene)-1,3- dihydro-isobenzofuran-5- ylmethyl]-pyrrolidin-3-yl}- acetamide | 407.443 |
| 216 | 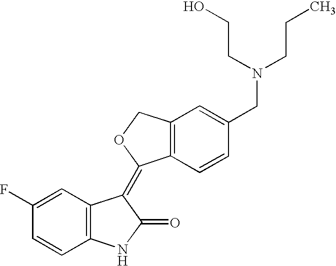 | 5-Fluoro-3-(5-{[(2-hydroxy- ethyl)-propyl-amino]-methyl}- 3H-isobenzofuran-1-ylidene)- 1,3-dihydro-indol-2-one | 382.433 |
| 217 | 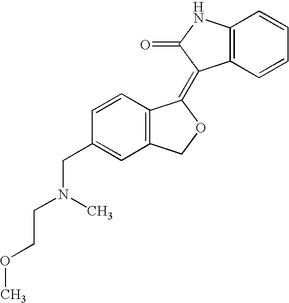 | 3-(5-{[(2-Methoxy-ethyl)- methyl-amino]-methyl}-3H- isobenzofuran-1-ylidene)-1,3- dihydro-indol-2-one | 350.416 |
| 218 | 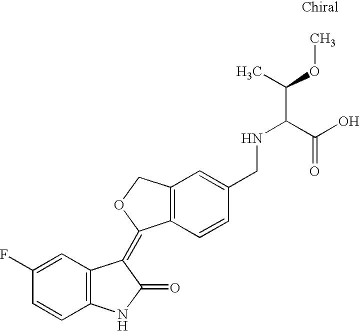 | (S)-2-{[1-(5-Fluoro-2-oxo-1,2- dihydro-indol-3-ylidene)-1,3- dihydro-isobenzofuran-5- ylmethyl]-amino}-(R)-3- methoxy-butyric acid | 412.415 |
| 219 | 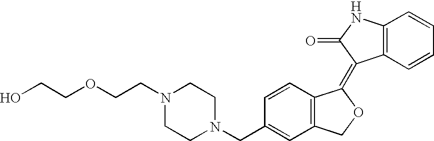 | 3-(5-{4-[2-(2-Hydroxy-ethoxy)- ethyl]-piperazin-1-ylmethyl}- 3H-isobenzofuran-1-ylidene)- 1,3-dihydro-indol-2-one | 435.521 |
| 220 | 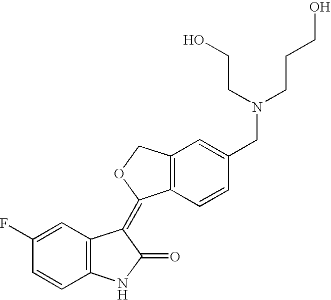 | 5-Fluoro-3-(5-{[(2-hydroxy- ethyl)-(3-hydroxy-propyl)- amino]-methyl}-3H- isobenzofuran-1-ylidene)-1,3- dihydro-indol-2-one | 398.432 |
| 221 | 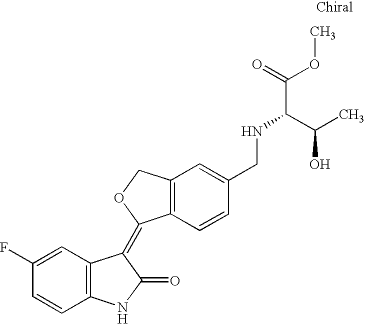 | (S)-2-{[1-(5-Fluoro-2-oxo-1,2- dihydro-indol-3-ylidene)-1,3- dihydro-isobenzofuran-5- ylmethyl]-amino}-(R)-3- hydroxy-butyric acid methyl ester | 412.415 |
| 222 | 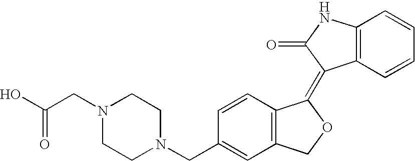 | {4-[1-(2-Oxo-1,2-dihydro- indol-3-ylidene)-1,3-dihydro- isobenzofuran-5-ylmethyl]- piperazin-1-yl}-acetic acid | 405.452 |
| 223 | 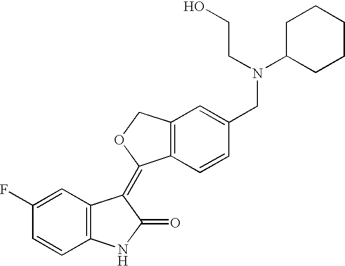 | 3-(5-{[Cyclohexyl-(2-hydroxy- ethyl)-amino]-methyl}-3H- isobenzofuran-1-ylidene)-5- fluoro-1,3-dihydro-indol-2-one | 422.497 |
| 224 | 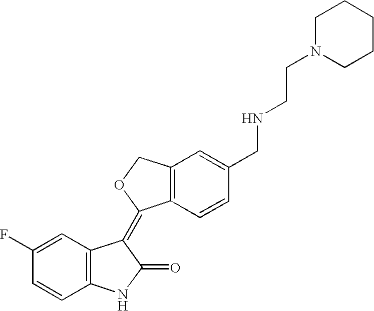 | 5-Fluoro-3-{5-[(2-piperidin-1- yl-ethylamino)-methyl]-3H- isobenzofuran-1-ylidene}-1,3- dihydro-indol-2-one | 407.486 |
| 225 | 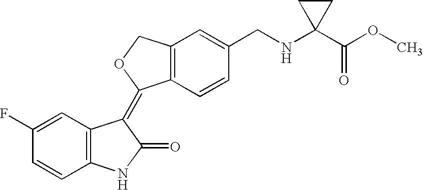 | 1-{[1-(5-Fluoro-2-oxo-1,2- dihydro-indol-3-ylidene)-1,3- dihydro-isobenzofuran-5- ylmethyl]-amino}cyclopropanecarboxylic acid methyl ester | 394.4 |
| 226 | 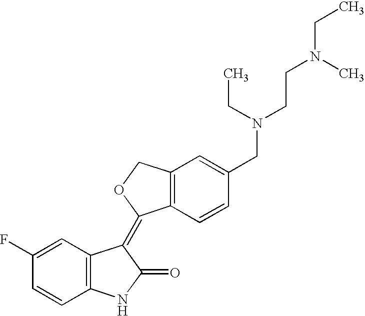 | 3-[5-({Ethyl-[2-(ethyl-methyl- amino)-ethyl]-amino}-methyl)- 3H-isobenzofuran-1-ylidene]- 5-fluoro-1,3-dihydro-indol-2- one | 409.502 |
| 227 | 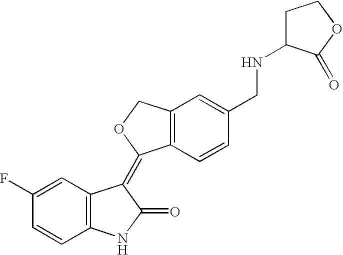 | 5-Fluoro-3-{5-[(2-oxo- tetrahydro-furan-3-ylamino)- methyl]-3H-isobenzofuran-1- ylidene}-1,3-dihydro-indol-2- one | 380.373 |
| 228 | 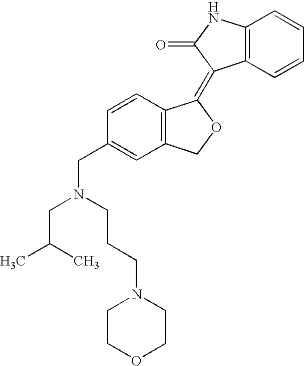 | 3-(5-{[Isobutyl-(3-morpholin-4- yl-propyl)-amino]-methyl}-3H- isobenzofuran-1-ylidene)-1,3- dihydro-indol-2-one | 461.603 |
| 229 | 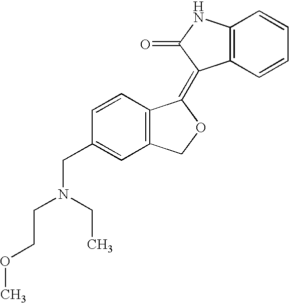 | 3-(5-{[Ethyl-(2-methoxy-ethyl)- amino]-methyl}-3H- isobenzofuran-1-ylidene)-1,3- dihydro-indol-2-one | 364.443 |
| 230 | 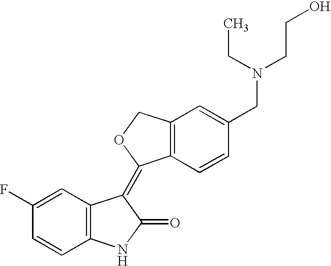 | 3-(5-{[Ethyl-(2-hydroxy-ethyl)- amino]-methyll-3H- isobenzofuran-1-ylidene)-5- fluoro-1,3-dihydro-indol-2-one | 368.406 |
| 231 | 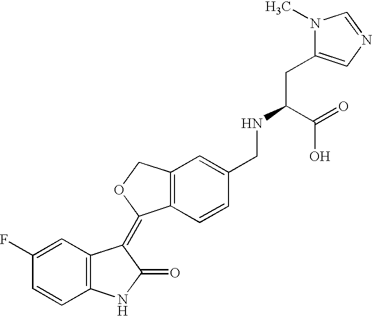 | (S)-2-{[1-(5-Fluoro-2-oxo-1,2- dihydro-indol-3-ylidene)-1,3- dihydro-isobenzofuran-5- ylmethyl]-aminol-3-(3-methyl- 3H-imidazol-4-yl)-propionic acid | 448.452 |
| 232 | 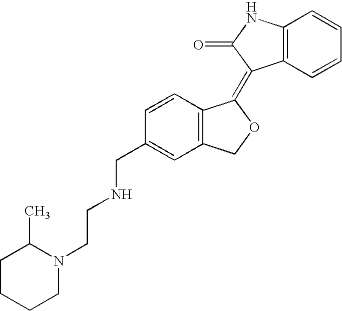 | 3-(5-{[2-(2-Methyl-piperidin-1- yl)-ethylaminol-methyl}-3H- isobenzofuran-1-ylidene)-1,3- dihydro-indol-2-one | 403.523 |
| 233 | 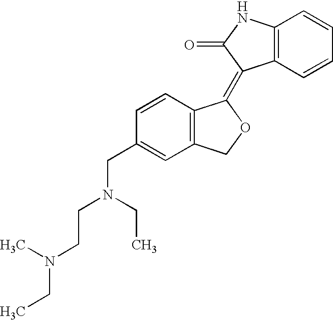 | 3-[5-({Ethyl-[2-(ethyl-methyl- amino)-ethyl]-amino}-methyl)- 3H-isobenzofuran-1-ylidene]- 1,3-dihydro-indol-2-one | 391.512 |
| 234 | 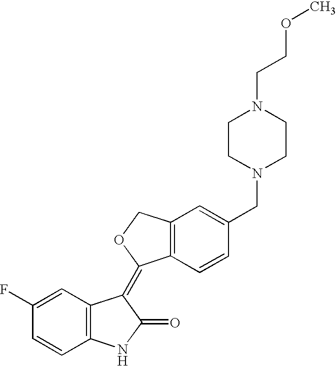 | 5-Fluoro-3-{5-(4-(2-methoxy- ethyl)-piperazin-1-ylmethyl]- 3H-isobenzofuran-1-ylidene}- 1,3-dihydro-indol-2-one | 423.485 |
| 235 | 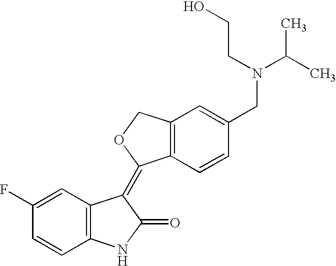 | 5-Fluoro-3-(5-{[(2-hydroxy- ethyl)-isopropyl-amino]- methyl}-3H-isobenzofuran-1- ylidene)-1,3-dihydro-indol-2- one | 382.433 |
| 236 | 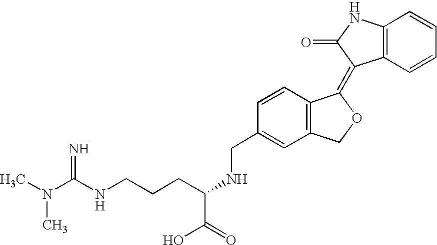 | 5-(N′,N′-Dimethyl-guanidino)- (S)-2-{[1-(2-oxo-1,2-dihydro- indol-3-ylidene)-1,3-dihydro- isobenzofuran-5-ylmethyl]- aminol-pentanoic acid | 463.535 |
| 237 | 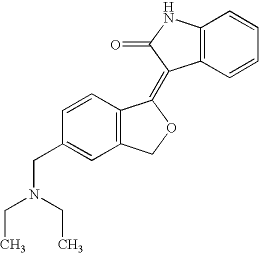 | 3-(5-Diethylaminomethyl-3H- isobenzofuran-1-ylidene)-1,3- dihydro-indol-2-one | 334.417 |
| 238 | 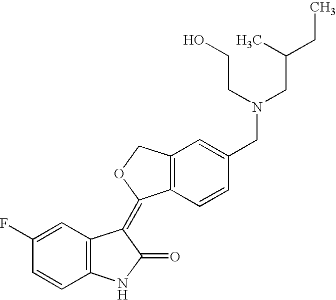 | 5-Fluoro-3-(5-{[(2-hydroxy- ethyl)-(2-methyl-butyl)-amino]- methyl}-3H-isobenzofuran-1- ylidene)-1,3-dihydro-indol-2- one | 410.486 |
| 239 | 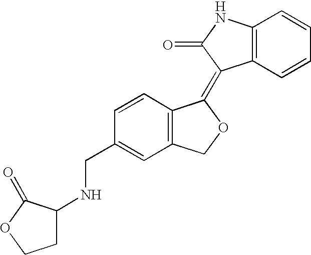 | 3-{5-[(2-Oxo-tetrahydro-furan- 3-ylamino)-methyl]-3H- isobenzofuran-1-ylidene}-1,3- dihydro-indol-2-one | 362.383 |
| 240 | 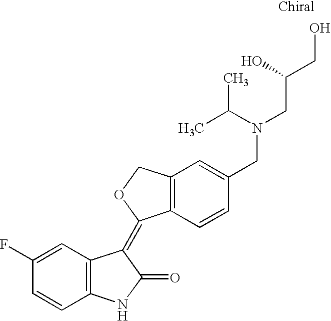 | 3-(5-{[((2S)-2,3-Dihydroxy- propyl)-isopropyl-amino]- methyl}-3H-isobenzofuran-1- ylidene)-5-fluoro-1,3-dihydro- indol-2-one | 412.458 |
| 241 | 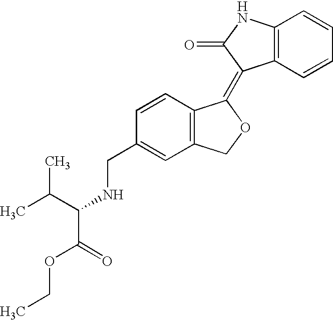 | 3-Methyl-(S)-2-{[1-(2-oxo-1,2- dihydro-indol-3-ylidene)-1,3- dihydro-isobenzofuran-5- ylmethyl]-amino}-butyric acid ethyl ester | 406.479 |
| 242 | 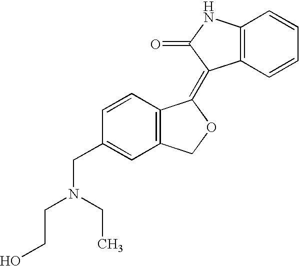 | 3-(5-{[Ethyl-(2-hydroxy-ethyl)- amino]-methyl}-3H- isobenzofuran-1-ylidene)-1,3- dihydro-indol-2-one | 350.416 |
| 243 | 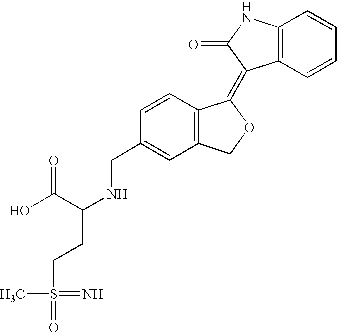 | 4-Methylsulfonimidosyl-2-{[1- (2-oxo-1,2-dihydro-indol-3- ylidene)-1,3-dihydro- isobenzofuran-5-ylmethyl]- amino}-butanoic acid | 441.506 |
| 244 | 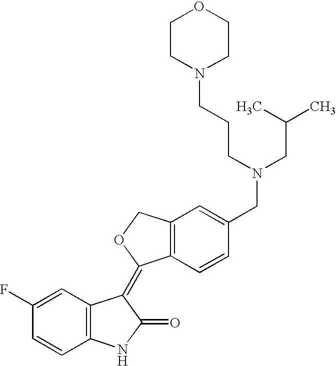 | 5-Fluoro-3-(5-{[isobutyl-(3- morpholin-4-yl-propyl)-amino]- methyl}-3H-isobenzofuran-1- ylidene)-1,3-dihydro-indol-2- one | 479.593 |
| 245 | 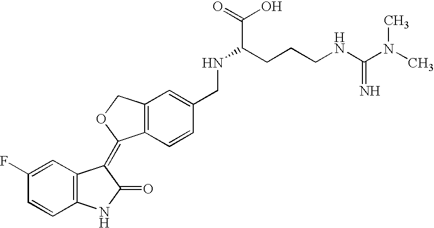 | 5-(N′, N′-Dimethyl-guanidino)- (S)-2-{[1-(5-fluoro-2-oxo-1,2- dihydro-indol-3-ylidene)-1,3- dihydro-isobenzofuran-5- ylmethyl]-amino}-pentanoic acid | 481.525 |
| 246 | 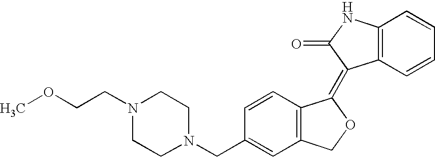 | 3-{5-[4-(2-Methoxy-ethyl)- piperazin-1-ylmethyl]-3H- isobenzofuran-1-ylidene}-1,3- dihydro-indol-2-one | 405.495 |
| 247 | 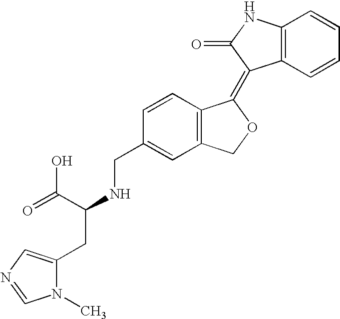 | 3-(3-Methyl-3H-imidazol-4-yl)- (S)-2-{[1-(2-oxo-1,2-dihydro- indol-3-ylidene)-1,3-dihydro- isobenzofuran-5-ylmethyl]- amino}-propionic acid | 430.462 |
| 248 | 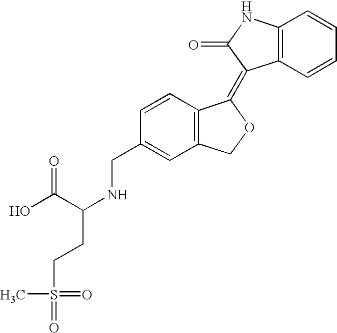 | 4-Methanesulfonyl-2-{[1-(2- oxo-1,2-dihydro-indol-3- ylidene)-1,3-dihydro- isobenzofuran-5-ylmethyl]- amino}-butyric acid | 442.49 |
| 249 | 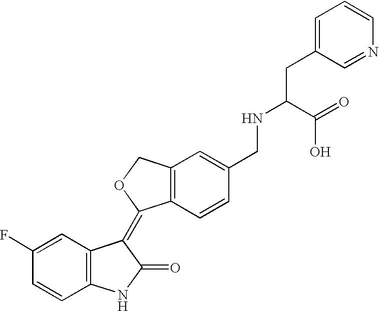 | 2-{[1-(5-Fluoro-2-oxo-1,2- dihydro-indol-3-ylidene)-1,3- dihydro-isobenzofuran-5- ylmethyl]-amino}-3-pyridin-3- yl-propionic acid | 445.448 |
| 250 | 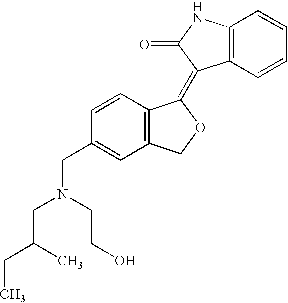 | 3-(5-{[(2-Hydroxy-ethyl)-(2- methyl-butyl)-amino]-methyl}- 3H-isobenzofuran-1-ylidene)- 1,3-dihydro-indol-2-one | 392.496 |
| 251 | 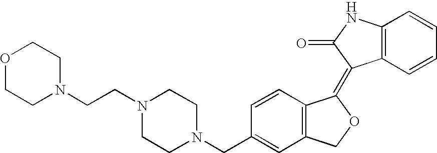 | 3-{5-[4-(2-Morpholin-4-yl- ethyl)-piperazin-1-ylmethyl]- 3H-isobenzofuran-1-ylidene}1,3-dihydro-indol-2-one | 460.575 |
| 252 | 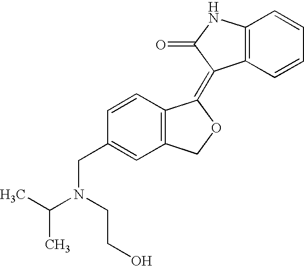 | 3-(5-{[(2-Hydroxy-ethyl)- isopropyl-amino]-methyl}-3H- isobenzofuran-1-ylidene)-1,3- dihydro-indol-2-one | 364.443 |
| 253 | 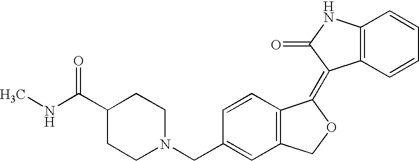 | 1-[1-(2-Oxo-1,2-dihydro-indol- 3-ylidene)-1,3-dihydro- isobenzofuran-5-ylmethyl]- piperidine-4-carboxylic acid methylamide | 403.479 |
| 254 | 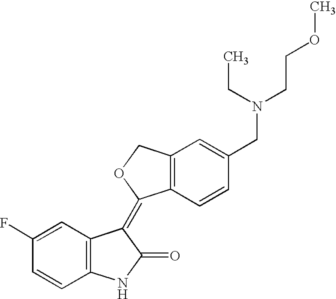 | 3-(5-{[Ethyl-(2-methoxy-ethyl)- amino]-methyl}-3H- isobenzofuran-1-ylidene)-5- fluoro-1,3-dihydro-indol-2-one | 382.433 |
| 255 | 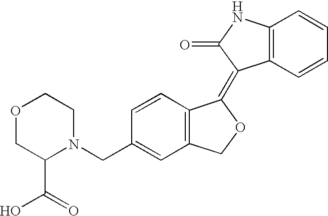 | 4-[1-(2-Oxo-1,2-dihydro-indol- 3-ylidene)-1,3-dihydro- isobenzofuran-5-ylmethyl]- morpholine-3-carboxylic acid | 392.409 |
| 256 | 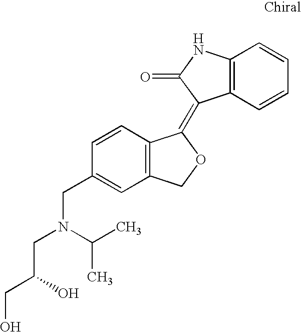 | 3-(5-{[((2R)-2,3-Dihydroxy- propyl)-isopropyl-amino]- methyl}-3H-isobenzofuran-1- ylidene)-1,3-dihydro-indol-2- one | 394.468 |
| 257 | 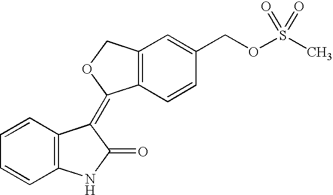 | Methanesulfonic acid 1-(2- oxo-1,2-dihydro-indol-3- ylidene)-1,3-dihydro- isobenzofuran-5-ylmethyl ester | 357.384 |
| 258 | 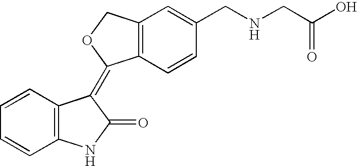 | {[1-(2-Oxo-1,2-dihydro-indol- 3-ylidene)-1,3-dihydro- isobenzofuran-5-ylmethyl]- amino}-acetic acid | 336.345 |
| 259 | 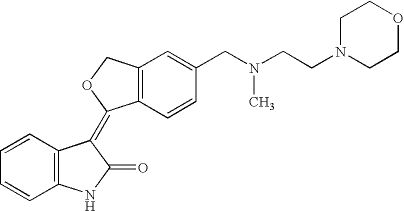 | 3-(5-{[Methyl-(2-morpholin-4- yl-ethyl)-amino]-methyl}-3H- isobenzofuran-1-ylidene)-1,3- dihydro-indol-2-one | 405.495 |
| 260 | 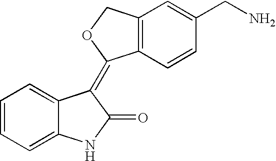 | 3-(5-Aminomethyl-3H- isobenzofuran-1-ylidene)-1,3- dihydro-indol-2-one | 278.31 |
| 261 | 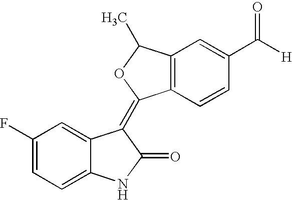 | 1-(5-Fluoro-2-oxo-1,2-dihydro- indol-3-ylidene)-3-methyl-1,3- dihydro-isobenzofuran-5- carbaldehyde | 309.295 |
| 262 | 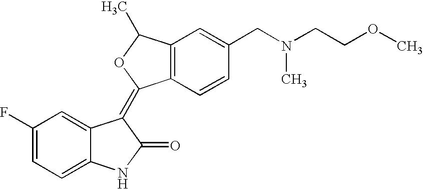 | 5-Fluoro-3-(5-{[(2-methoxy ethyl)-methyl-amino]-methyl}- 3-methyl-3H-isobenzofuran-1- ylidene)-1,3-dihydro-indol-2- one | 382.433 |
| 263 | 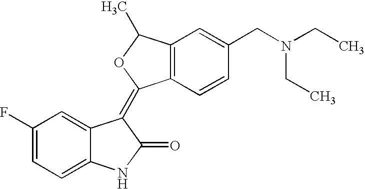 | 3-(5-Diethylaminomethyl-3- methyl-3H-isobenzofuran-1- ylidene)-5-fluoro-1,3-dihydro- indol-2-one | 366.434 |
| 264 | 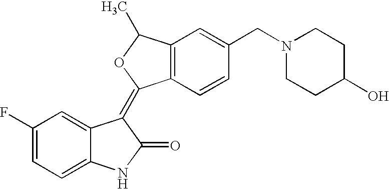 | 5-Fluoro-3-[5-(4-hydroxy- piperidin-1-ylmethyl)-3-methyl- 3H-isobenzofuran-1-ylidene]- 1,3-dihydro-indol-2-one | 394.444 |
| 265 | 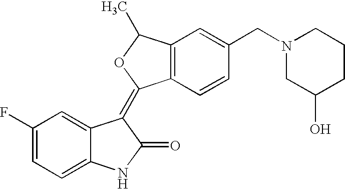 | 5-Fluoro-3-[5-(3-hydroxy- piperidin-1-ylmethyl)-3-methyl- 3H-isobenzofuran-1-ylidene]- 1,3-dihydro-indol-2-one | 394.444 |
| 266 | 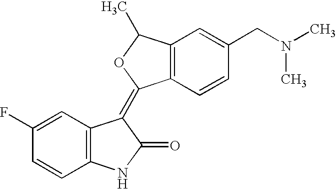 | 3-(5-Dimethylaminomethyl-3- methyl-3H-isobenzofuran-1- ylidene)-5-fluoro-1,3-dihydro- indol-2-one | 338.38 |
| 267 | 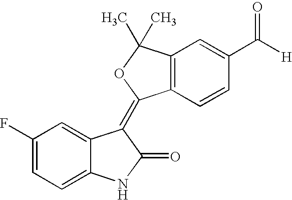 | 1-(5-Fluoro-2-oxo-1,2-dihydro- indol-3-ylidene)-3,3-dimethyl- 1,3-dihydro-isobenzofuran-5- carbaldehyde | 323.322 |
| 268 | 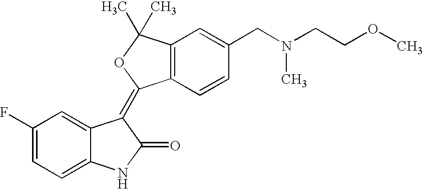 | 5-Fluoro-3-(5-{[(2-methoxy- ethyl)-methyl-amino]-methyl}- 3,3-dimethyl-3H- isobenzofuran-1-ylidene)-1,3- dihydro-indol-2-one | 396.46 |
| 269 | 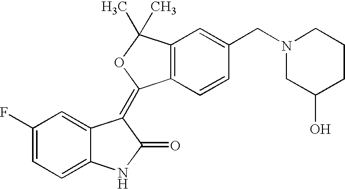 | 5-Fluoro-3-[5-(3-hydroxy- piperidin-1-ylmethyl)-3,3- dimethyl-3H-isobenzofuran-1- ylidene]-1,3-dihydro-indol-2- one | 408.47 |
| 270 | 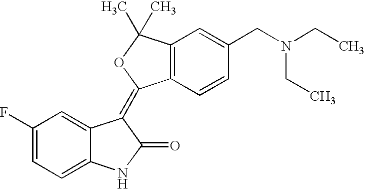 | 3-(5-Diethylaminomethyl-3,3- dimethyl-3H-isobenzofuran-1- ylidene)-5-fluoro-1,3-dihydro- indol-2-one | 380.461 |
| 271 | 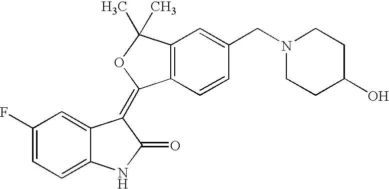 | 5-Fluoro-3-[5-(4-hydroxy- piperidin-1-ylmethyl)-3,3- dimethyl-3H-isobenzofuran-1- ylidene]-1,3-dihydro-indol-2- one | 408.47 |
| 272 | 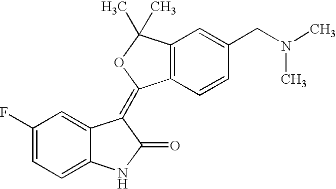 | 3-(5-Dimethylaminomethyl- 3,3-dimethyl-3H- isobenzofuran-1-ylidene)-5- fluoro-1,3-dihydro-indol-2-one | 352.407 |
| 273 | 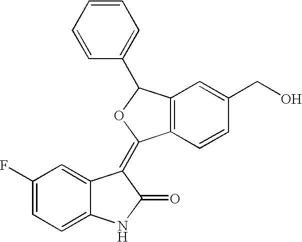 | 5-Fluoro-3-(5-hydroxymethyl- 3-phenyl-3H-isobenzofuran-1- ylidene)-1,3-dihydro-indol-2- one | 373.381 |
| 274 | 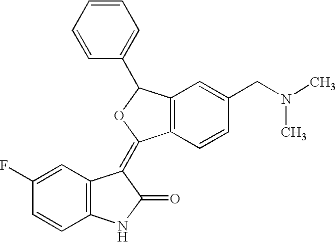 | 3-(5-Dimethylaminomethyl-3- phenyl-3H-isobenzofuran-1- ylidene)-5-fluoro-1,3-dihydro- indol-2-one | 400.451 |

TABLE 8

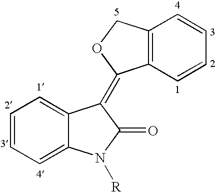

| Example | | | Molecular |
|---|---|---|---|
| Number | Chemical Structure | Chemical Name | Weight |
| 275 | 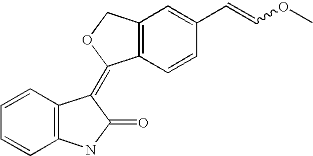 | 3-[5-(2-Methoxy-vinyl)-3H- isobenzofuran-1-ylidene]-1,3- dihydro-indol-2-one | 305.332 |
| 276 | 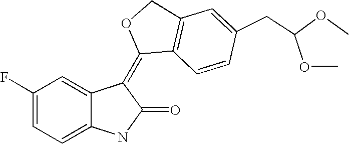 | 3-[5-(2,2-Dimethoxy-ethyl)- 3H-isobenzofuran-1-ylidene]- 1,3-dihydro-indol-2-one | 337.373 |
| 277 | 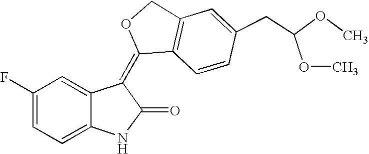 | 3-[5-(2,2-Dimethoxy-ethyl)- 3H-isobenzofuran-1-ylidene]- 5-fluoro-1,3-dihydro-indol-2- one | 355.363 |
| 278 | 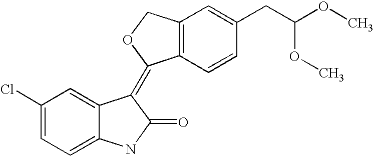 | 5-Chloro-3-[5-(2,2-dimethoxy- ethyl)-3H-isobenzofuran-1- ylidene]-1,3-dihydro-indol-2- one | 371.818 |
| 279 | 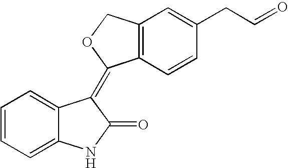 | [1-(2-Oxo-1,2-dihydro-indol-3- ylidene)-1,3-dihydro- isobenzofuran-5-yl]- acetaldehyde | 291.305 |
| 280 | 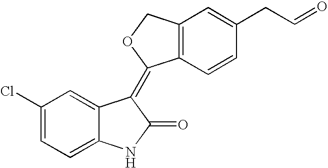 | [1-(5-Chloro-2-oxo-1,2- dihydro-indol-3-ylidene)-1,3- dihydro-isobenzofuran-5-yl]- acetaldehyde | 325.75 |
| 281 | 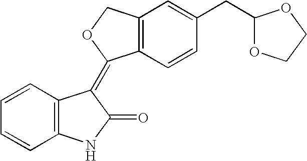 | 3-(5-[1,3]Dioxolan-2-ylmethyl- 3H-isobenzofuran-1-ylidene)- 1,3-dihydro-indol-2-one | 335.357 |
| 282 | 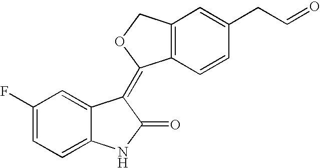 | [1-(5-Fluoro-2-oxo-1,2- dihydro-indol-3-ylidene)-1,3- dihydro-isobenzofuran-5-yl]- acetaldehyde | 309.295 |
| 283 | 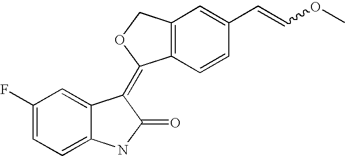 | 5-Fluoro-3-[5-(2-methoxy- vinyl)-3H-isobenzofuran-1- ylidene]-1,3-dihydro-indol-2- one | 323.322 |
| 284 | 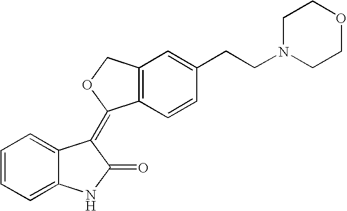 | 3-[5-(2-Morpholin-4-yl-ethyl)- 3H-isobenzofuran-1-ylidene]- 1,3-dihydro-indol-2-one | 362.427 |
| 285 | 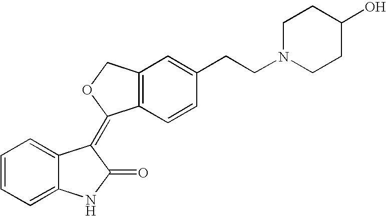 | 3-{5-[2-(4-Hydroxy-piperidin-1- yl)-ethyl]-3H-isobenzofuran-1- ylidene}-1,3-dihydro-indol-2- one | 376.454 |
| 286 | 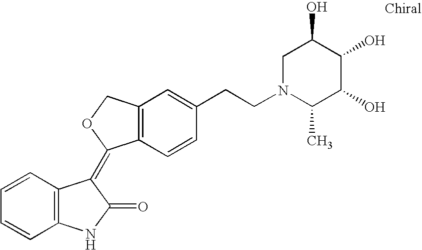 | 3-{5-[2-(3,4,5-Trihydroxy-2- methylpiperidin-1-yl) ethyl]- 3H-isobenzofuran-1-ylidene}- 1,3-dihydro-indol-2-one | 422.478 |
| 287 | 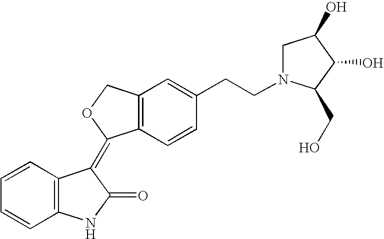 | 3-{5-[2-(3,4-Dihydroxy-2- hydroxymethyl-pyrrolidin-1-yl)- ethyl]-3H-isobenzofuran-1- ylidene}-1,3-dihydro-indol-2- one | 408.452 |
| 288 | 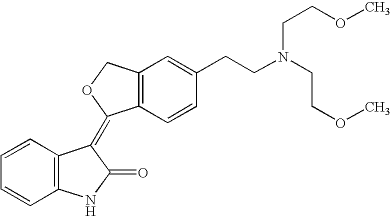 | 3-(5-{2-(Bis-(2-methoxy-ethyl)- amino]-ethyl}-3H- isobenzofuran-1-ylidene)-1,3- dihydro-indol-2-one | 408.495 |
| 289 | 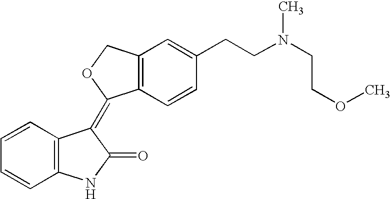 | 3-(5-{2-[(2-Methoxy-ethyl)- methyl-amino]-ethyl}-3H- isobenzofuran-1-ylidene)-1,3- dihydro-indol-2-one | 364.443 |
| 290 | 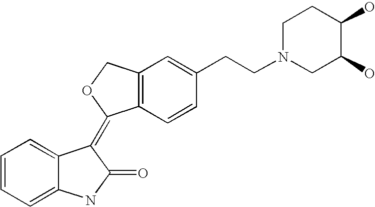 | cis-3-{5-[2-(3,4-Dihydroxy- piperidin-1-yl)ethyl]-3H- isobenzofuran-1-ylidene}-1,3- dihydro-indol-2-one | 392.453 |
| 291 | 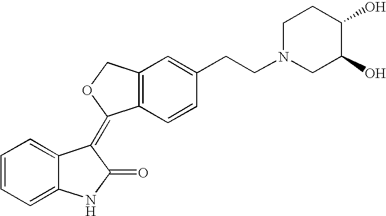 | trans-3-{5-[2-(3,4-Dihydroxy- piperidin-1-yl)-ethyl]-3H- isobenzofuran-1-ylidene}-1,3- dihydro-indol-2-one | 392.453 |
| 292 | 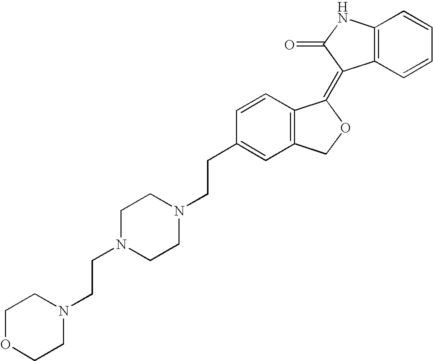 | 3-(5-{2-[4-(2-Morpholin-4-yl- ethyl)-piperazin-1-yl]-ethyl}- 3H-isobenzofuran-1-ylidene)- 1,3-dihydro-indol-2-one | |
| 293 | 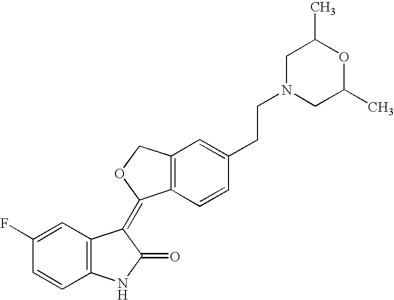 | 3-{5-[2-(2,6-Dimethyl- morpholin-4-yl)-ethyl]-3H- isobenzofuran-1-ylidene}-5- fluoro-1,3-dihydro-indol-2-one | 408.47 |
| 294 | 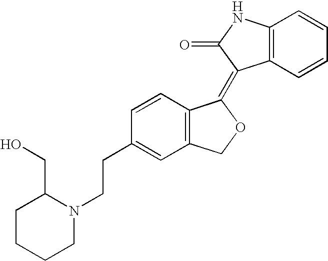 | 3-{5-[2-(2-Hydroxymethyl- piperidin-1-yl)-ethyl]-3H- isobenzofuran-1-ylidene}-1,3- dihydro-indol-2-one | 390.48 |
| 295 | 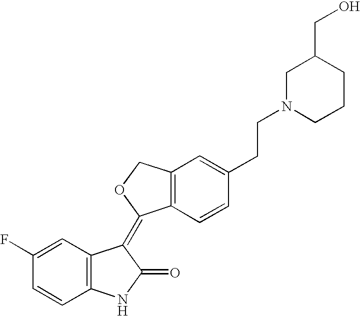 | 5-Fluoro-3-{5-[2-(3- hydroxymethyl-piperidin-1-yl)- ethyl]-3H-isobenzofuran-1- ylidene}-1,3-dihydro-indol-2- one | 408.47 |
| 296 | 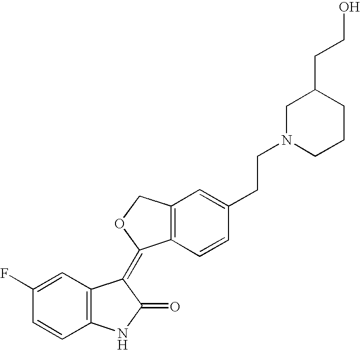 | 5-Fluoro-3-(5-{2-[3-(2- hydroxy-ethyl)-piperidin-1-yl]- ethyl}-3H-isobenzofuran-1- ylidene)-1,3-dihydro-indol-2- one | 422.497 |
| 297 | 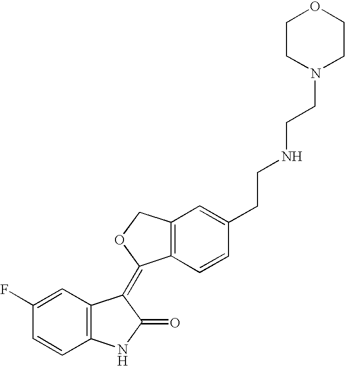 | 5-Fluoro-3-{5-[2-(2-morpholin- 4-yl-ethylamino)-ethyl]-3H- isobenzofuran-1-ylidene}-1,3- dihydro-indol-2-one | 423.485 |
| 298 | 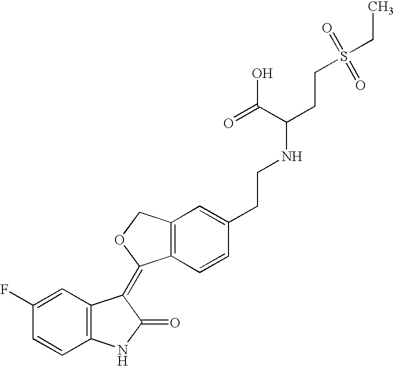 | 4-Ethanesulfonyl-2-{2-[1-(5- fluoro-2-oxo-1,2-dihydro-indol- 3-ylidene)-1,3-dihydro- isobenzofuran-5-yl]- ethylamino}-butyric acid | 488.534 |
| 299 | 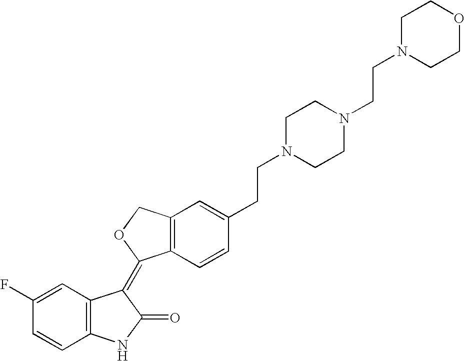 | 5-Fluoro-3-(5-{2-[4-(2- morpholin-4-yl-ethyl)- piperazin-1-yl]-ethyl}-3H- isobenzofuran-1-ylidene)-1,3- dihydro-indol-2-one | 492.592 |
| 300 | 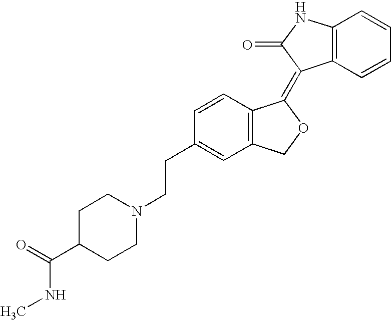 | 1-{2-[1-(2-Oxo-1,2-dihydro- indol-3-ylidene)-1,3-dihydro- isobenzofuran-5-yl]-ethyl}- piperidine-4-carboxylic acid methylamide | 417.506 |
| 301 | 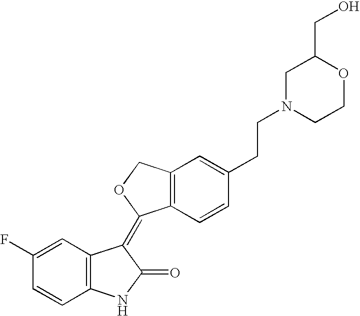 | 5-Fluoro-3-{5-(2-(2- hydroxymethyl-morpholin-4- yl)-ethyl]-3H-isobenzofuran-1- ylidene}-1,3-dihydro-indol-2- one | 410.443 |
| 302 | 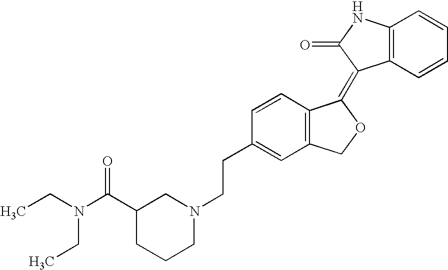 | 1-{2-[1-(2-Oxo-1,2-dihydro- indol-3-ylidene)-1,3-dihydro- isobenzofuran-5-yl]-ethyl}- piperidine-3-carboxylic acid diethylamide | 459.587 |
| 303 | 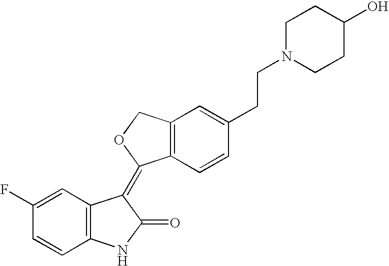 | 5-Fluoro-3-{5-[2-(4-hydroxy- piperidin-1-yl)-ethyl]-3H- isobenzofuran-1-ylidene}-1,3- dihydro-indol-2-one | 394.444 |
| 304 | 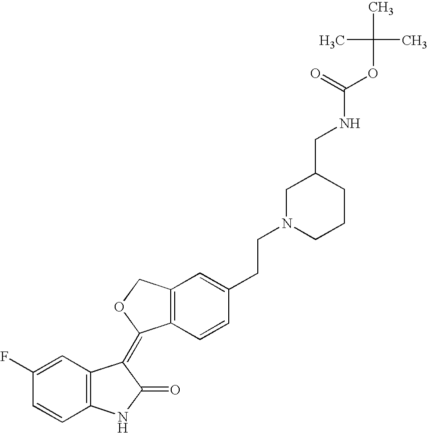 | (1-{2-[1-(5-Fluoro-2-oxo-1,2- dihydro-indol-3-ylidene)-1,3- dihydro-isobenzofuran-5-yl]- ethyl}-piperidin-3-ylmethyl)- carbamic acid tert-butyl ester | 507.603 |
| 305 | 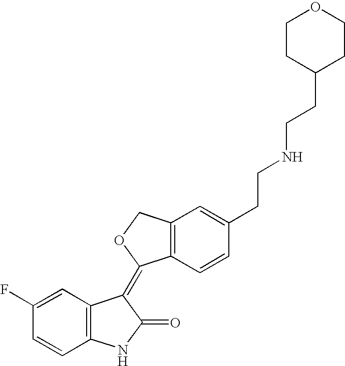 | 5-Fluoro-3-(5-{2-[2- (tetrahydro-pyran-4-yl)- ethylamino]-ethyl}-3H- isobenzofuran-1-ylidene)-1,3- dihydro-indol-2-one | 422.497 |
| 306 | 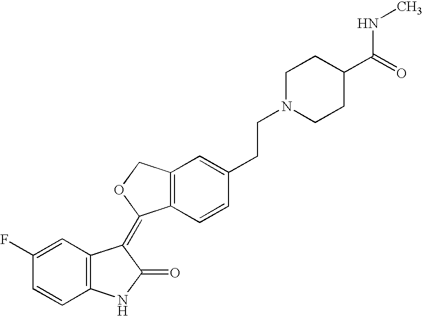 | 1-{2-[1-(5-Fluoro-2-oxo-1,2- dihydro-indol-3-ylidene)-1,3- dihydro-isobenzofuran-5-yl]- ethyl}-piperidine-4-carboxylic acid methylamide | 435.496 |
| 307 | 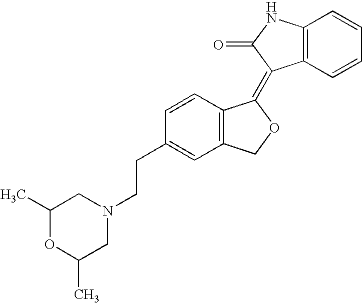 | 3-{5-[2-(2,6-Dimethyl- morpholin-4-yl)-ethyl]-3H- isobenzofuran-1-ylidene}-1,3- dihydro-indol-2-one | 390.48 |
| 308 | 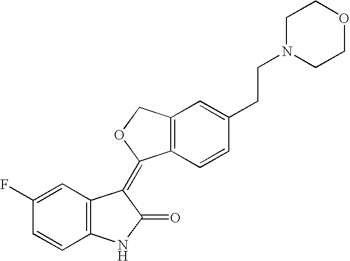 | 5-Fluoro-3-[5-(2-morpholin-4- yl-ethyl)-3H-isobenzofuran-1- ylidene]-1,3-dihydro-indol-2- one | 380.417 |
| 309 | 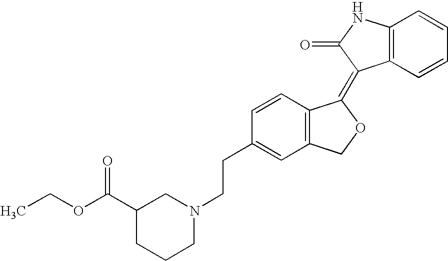 | 1-{2-[1-(2-Oxo-1,2-dihydro- indol-3-ylidene)-1,3-dihydro- isobenzofuran-5-yl]-ethyl}- piperidine-3-carboxylic acid ethyl ester | 432.517 |
| 310 | 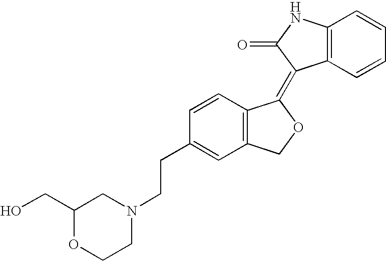 | 3-{5-[2-(2-Hydroxymethyl- morpholin-4-yl)-ethyl]-3H- isobenzofuran-1-ylidene}-1,3- dihydro-indol-2-one | 392.453 |
| 311 | 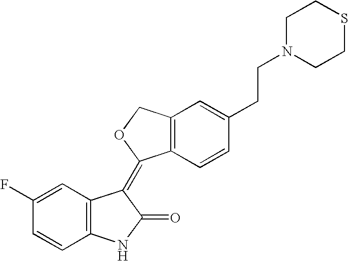 | 5-Fluoro-3-[5-(2- thiomorpholin-4-yl-ethyl)-3H- isobenzofuran-1-ylidene]-1,3- dihydro-indol-2-one | 396.484 |
| 312 | 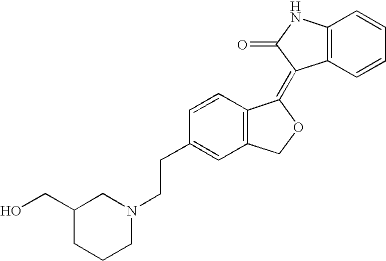 | 3-{5-[2-(3-Hydroxymethyl- piperidin-1-yl)-ethyl]-3H- isobenzofuran-1-ylidene}-1,3- dihydro-indol-2-one | 390.48 |
| 313 | 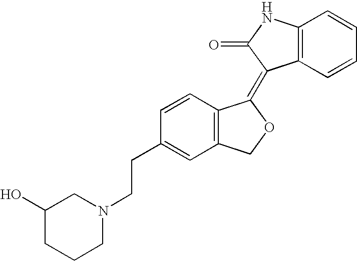 | 3-{5-[2-(3-Hydroxy-piperidin-1- yl)-ethyl]-3H-isobenzofuran-1- ylidene}-1,3-dihydro-indol-2- one | 376.454 |
| 314 | 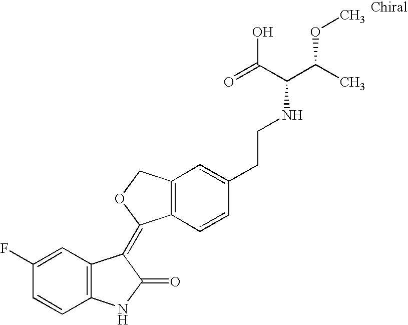 | 2-{(R)-2-[1-(5-Fluoro-2-oxo- 1,2-dihydro-indol-3-ylidene)- 1,3-dihydro-isobenzofuran-5- yl]-ethylamino}-(R)-3- methoxy-butyric acid | 426.442 |
| 315 | 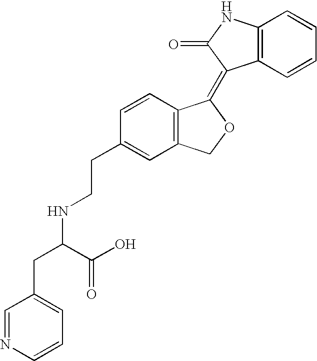 | 2-{2-[1-(2-Oxo-1,2-dihydro- indol-3-ylidene)-1,3-dihydro- isobenzofuran-5-yl]- ethylamino}-3-pyridin-3-yl- propionic acid | 441.485 |
| 316 | 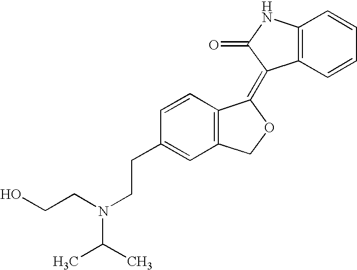 | 3-(5-{2-[(2-Hydroxy-ethyl)- isopropyl-amino]-ethyl}-3H- isobenzofuran-1-ylidene)-1,3- dihydro-indol-2-one | 378.469 |
| 317 | 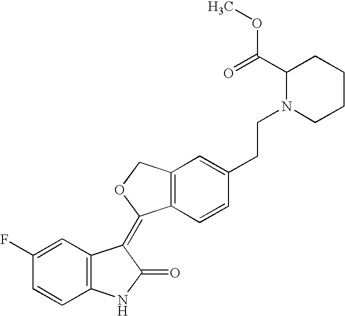 | 1-{2-[1-(5-Fluoro-2-oxo-1,2- dihydro-indol-3-ylidene)-1,3- dihydro-isobenzofuran-5-yl]- ethyl}-piperidine-2-carboxylic acid methyl ester | 436.48 |
| 318 | 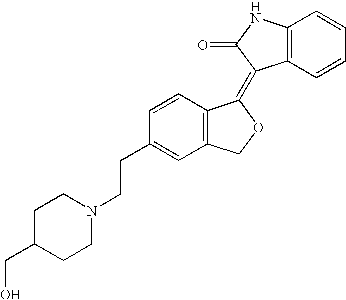 | 3-{5-[2-(4-Hydroxymethyl- piperidin-1-yl)-ethyl]-3H- isobenzofuran-1-ylidene}-1,3- dihydro-indol-2-one | 390.48 |
| 319 | 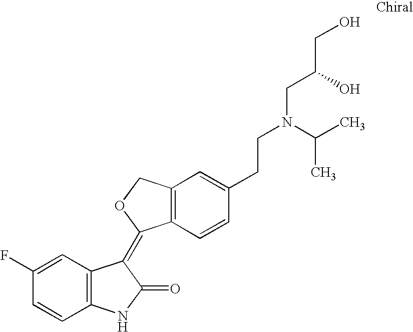 | 3-[5-(2-{[(2S)-2,3-Dihydroxy- propyl]-isopropyl-amino}- ethyl)-3H-isobenzofuran-1- ylidene]-5-fluoro-1,3-dihydro- indol-2-one | 426.485 |
| 320 | 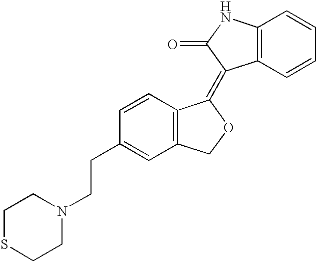 | 3-[5-(2-Thiomorpholin-4-yl- ethyl)-3H-isobenzofuran-1- ylidene]-1,3-dihydro-indol-2- one | 378.494 |
| 321 | 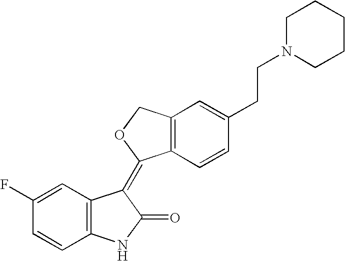 | 5-Fluoro-3-[5-(2-piperidin-1-yl- ethyl)-3H-isobenzofuran-1- ylidene]-1,3-dihydro-indol-2- one | 378.445 |
| 322 | 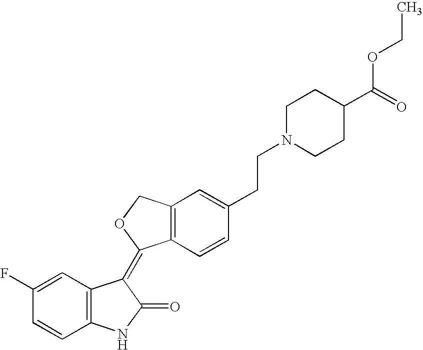 | 1-{2-[1-(5-Fluoro-2-oxo-1,2- dihydro-indol-3-ylidene)-1,3- dihydro-isobenzofuran-5-yl]- ethyl}-piperidine-4-carboxylic acid ethyl ester | 450.507 |
| 323 | 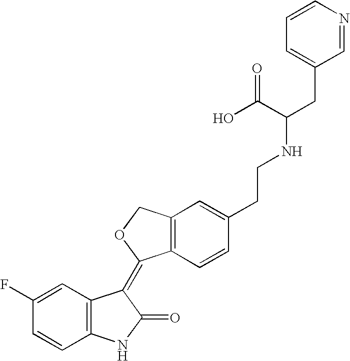 | 2-{2-[1-(5-Fluoro-2-oxo-1,2- dihydro-indol-3-ylidene)-1,3- dihydro-isobenzofuran-5-yl]- ethylamino}-3-pyridin-3-yl- propionic acid | 459.475 |
| 324 | 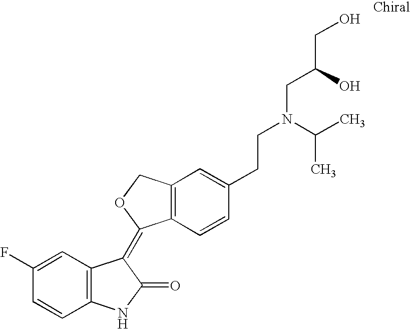 | 3-[5-(2-{[(2R)-2,3-Dihydroxy- propyl]-isopropyl-amino}- ethyl)-3H-isobenzofuran-1- ylidene]-5-fluoro-1,3-dihydro- indol-2-one | 426.485 |
| 325 | 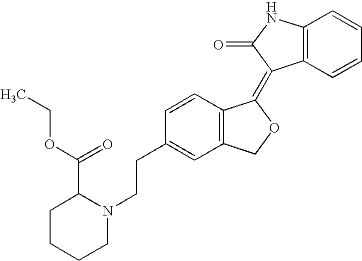 | 1-{2-[1-(2-Oxo-1,2-dihydro- indol-3-ylidene)-1,3-dihydro- isobenzofuran-5-yl]-ethyl}- piperidine-2-carboxylic acid ethyl ester | 432.517 |
| 326 | 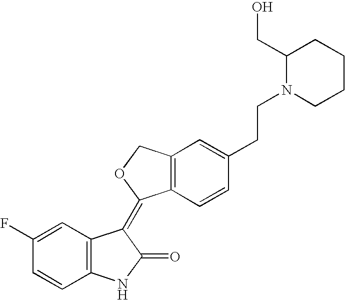 | 5-Fluoro-3-{5-[2-(2- hydroxymethyl-piperidin-1-yl)- ethyl]-3H-isobenzofuran-1- ylidene}-1,3-dihydro-indol-2- one | 408.47 |
| 327 | 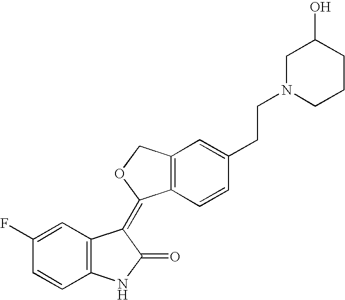 | 5-Fluoro-3-{5-[2-(3-hydroxy- piperidin-1-yl)-ethyl]-3H- isobenzofuran-1-ylidene}-1,3- dihydro-indol-2-one | 394.444 |
| 328 | 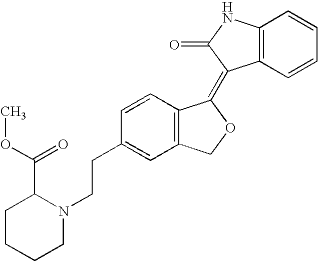 | 1-{2-[1-(2-Oxo-1,2-dihydro- indol-3-ylidene)-1,3-dihydro- isobenzofuran-5-yl]-ethyl}- piperidine-2-carboxylic acid methyl ester | 418.49 |
| 329 | 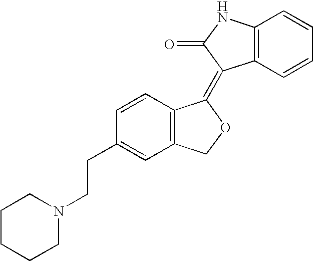 | 3-[5-(2-Piperidin-1-yl-ethyl)- 3H-isobenzofuran-1-ylidene]- 1,3-dihydro-indol-2-one | 360.455 |
| 330 | 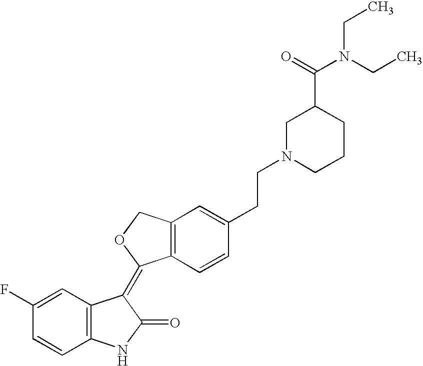 | 1-{2-[1-(5-Fluoro-2-oxo-1,2- dihydro-indol-3-ylidene)-1,3- dihydro-isobenzofuran-5-yl]- ethyl}-piperidine-3-carboxylic ethyl}-piperidine-3-carboxylic acid diethylamide | 477.577 |
| 331 | 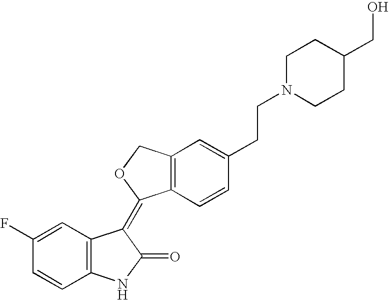 | 5-Fluoro-3-{5-[2-(4- hydroxymethyl-piperidin-1-yl)- ethyl]-3H-isobenzofuran-1- ylidene}-1,3-dihydro-indol-2- one | 408.47 |
| 332 | 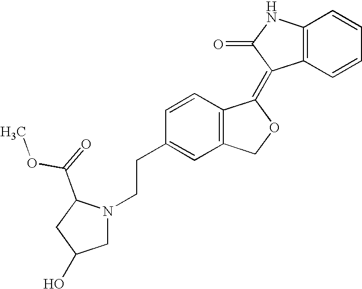 | 4-Hydroxy-1-{2-[1-(2-oxo-1,2- dihydro-indol-3-ylidene)-1,3- dihydro-isobenzofuran-5-yl]- ethyl}-pyrrolidine-2-carboxylic acid methyl ester | 420.463 |
| 333 | 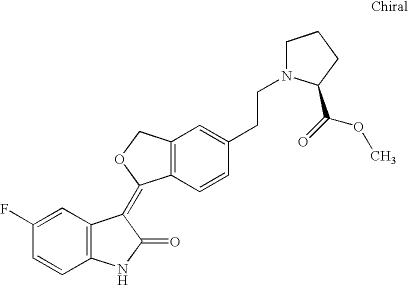 | 1-{2-[1-(5-Fluoro-2-oxo-1,2- dihydro-indol-3-ylidene)-1,3- dihydro-isobenzofuran-5-yl]- ethyl}-pyrrolidine-(S)-2- carboxylic acid methyl ester | 422.454 |
| 334 | 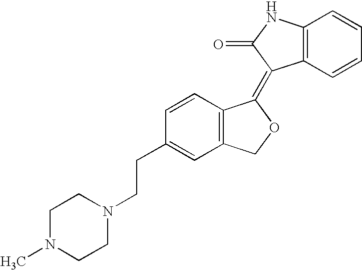 | 3-{5-[2-(4-Methyl-piperazin-1- yl)-ethyl]-3H-isobenzofuran-1- ylidene}-1,3-dihydro-indol-2- one | 375.47 |
| 335 | 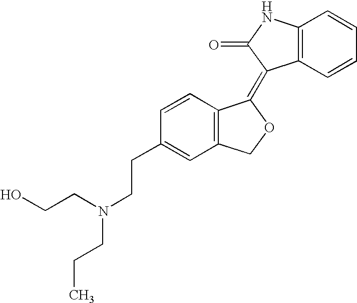 | 3-(5-{2-[(2-Hydroxy-ethyl)- propyl-amino]-ethyl}-3H- isobenzofuran-1-ylidene)-1,3- dihydro-indol-2-one | 378.469 |
| 336 | 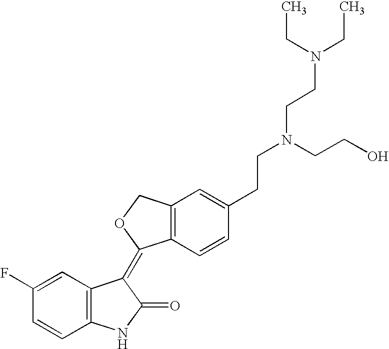 | 3-(5-{2-[(2-Diethylamino- ethyl)-(2-hydroxy-ethyl)- amino]-ethyl}-3H- isobenzofuran-1-ylidene)-5- fluoro-1,3-dihydro-indol-2-one | 453.555 |
| 337 | 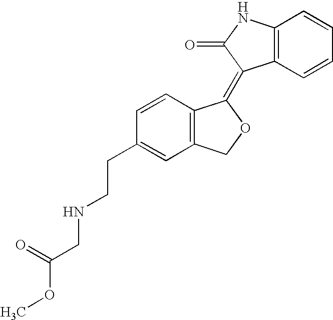 | {2-[1-(2-Oxo-1,2-dihydro- indol-3-ylidene)-1,3-dihydro- isobenzofuran-5-yl]- ethylamino}-acetic acid methyl ester | 364.399 |
| 338 | 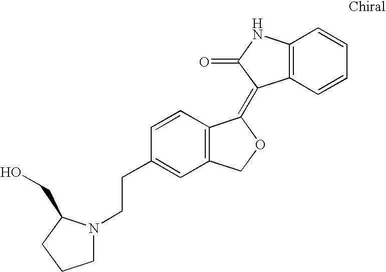 | 3-(5-{2-[(S)-2-Hydroxymethyl- pyrrolidin-1-yl]-ethyl}-3H- isobenzofuran-1-ylidene)-1,3- dihydro-indol-2-one | 376.454 |
| 339 | 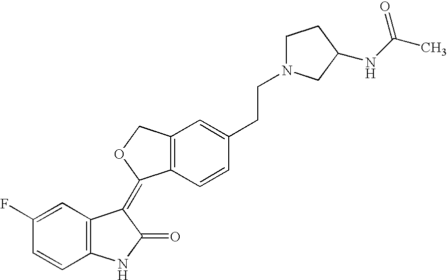 | N-(1-{2-[1-(5-Fluoro-2-oxo- 1,2-dihydro-indol-3-ylidene)- 1,3-dihydro-isobenzofuran-5- yl]-ethyl}-pyrrolidin-3-yl)- acetamide | 421.47 |
| 340 | 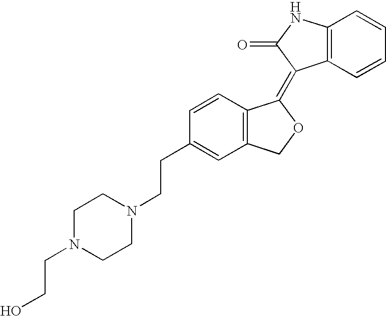 | 3-(5-{2-[4-(2-Hydroxy-ethyl)- piperazin-1-yl]-ethyl}-3H- isobenzofuran-1-ylidene)-1,3- dihydro-indol-2-one | 405.495 |
| 341 | 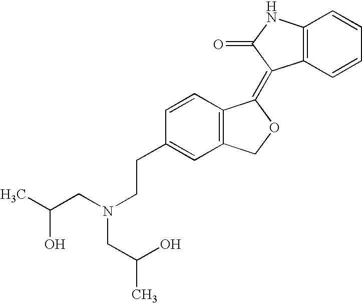 | 3-(5-{2-[Bis-(2-hydroxy- propyl)-amino]-ethyl}-3H- isobenzofuran-1-ylidene)-1,3- dihydro-indol-2-one | 408.495 |
| 342 | 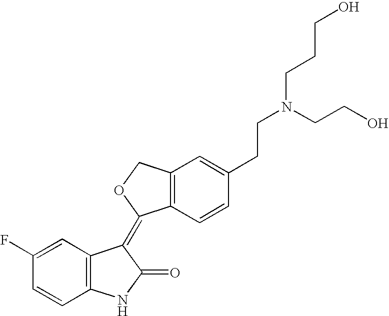 | 5-Fluoro-3-(5-{2-[(2-hydroxy- ethyl)-(3-hydroxy-propyl)- amino]-ethyl}-3H- isobenzofuran-1-ylidene)-1,3- dihydro-indol-2-one | 412.458 |
| 343 | 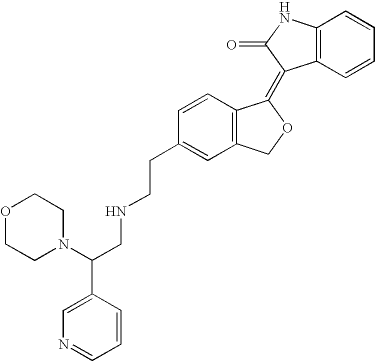 | 3-{5-[2-(2-Morpholin-4-yl-2- pyridin-3-yl-ethylamino)-ethyl]- 3H-isobenzofuran-1-ylidene}- 1,3-dihydro-indol-2-one | 482.581 |
| 344 | 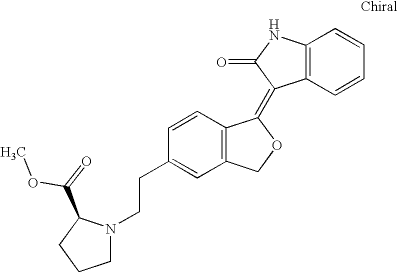 | 1-{2-[1-(2-Oxo-1,2-dihydro- indol-3-ylidene)-1,3-dihydro- isobenzofuran-5-yl]-ethyl}- pyrrolidine-(S)-2-carboxylic acid methyl ester | 404.464 |
| 345 | 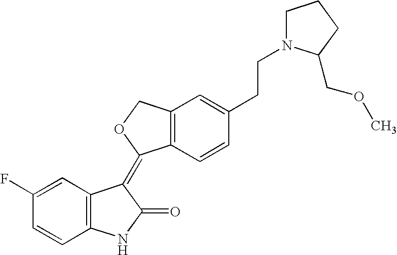 | 5-Fluoro-3-{5-[2-(2- methoxymethyl-pyrrolidin-1- yl)-ethyl]-3H-isobenzofuran-1- ylidene}-1,3-dihydro-indol-2- one | 408.47 |
| 346 | 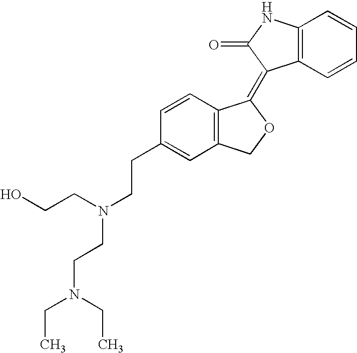 | 3-(5-{2-[(2-Diethylamino- ethyl)-(2-hydroxy-ethyl)- amino]-ethyl}-3H- isobenzofuran-1-ylidene)-1,3- dihydro-indol-2-one | 435.565 |
| 347 | 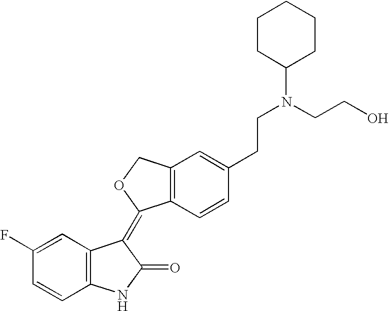 | 3-(5-{2-[Cyclohexyl-(2- hydroxy-ethyl)-amino]-ethyl}- 3H-isobenzofuran-1-ylidene)- 5-fluoro-1,3-dihydro-indol-2- one | 436.524 |
| 348 | 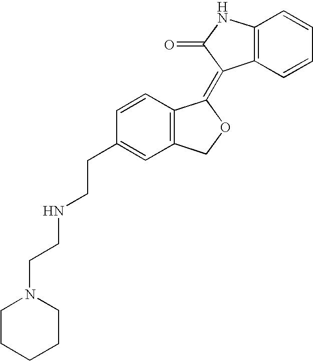 | 3-{5-[2-(2-Piperidin-1-yl- ethylamino)-ethyl]-3H- isobenzofuran-1-ylidene}-1,3- dihydro-indol-2-one | 403.523 |
| 349 | 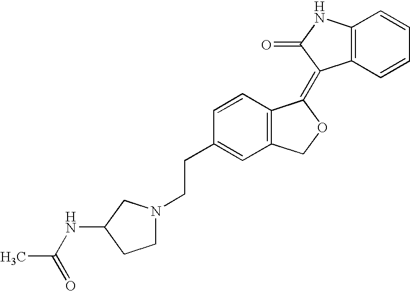 | N-(1-{2-[1-(2-Oxo-1,2-dihydro- indol-3-ylidene)-1,3-dihydro- isobenzofuran-5-yl]-ethyl}- pyrrolidin-3-yl)-acetamide | 403.479 |
| 350 | 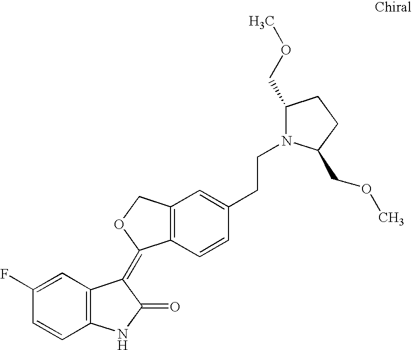 | 3-(5-{2-[(S,S)-2,5-Bis- methoxymethyl-pyrrolidin-1- yl]-ethyl}-3H-isobenzofuran-1- ylidene)-5-fluoro-1,3-dihydro- indol-2-one | 452.523 |
| 351 | 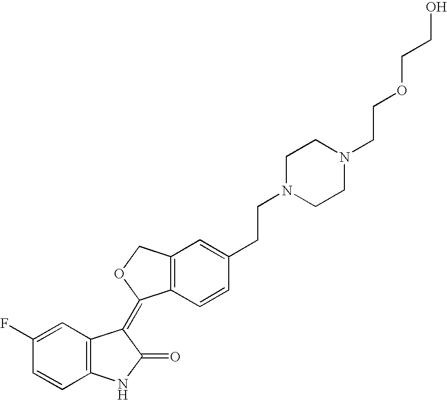 | 5-Fluoro-3-[5-(2-{4-[2-(2- hydroxy-ethoxy)-ethyl]- piperazin-1-yl}-ethyl)-3H- isobenzofuran-1-ylidene]-1,3- dihydro-indol-2-one | 467.538 |
| 352 | 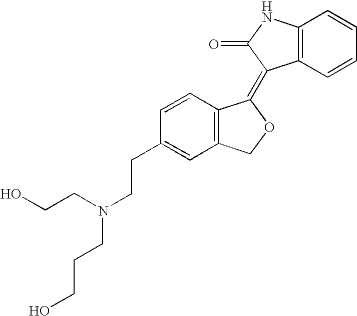 | 3-(5-{2-[(2-Hydroxy-ethyl)-(3- hydroxy-propyl)-amino]-ethyl}- 3H-isobenzofuran-1-ylidene)- 1,3-dihydro-indol-2-one | 394.468 |
| 353 | 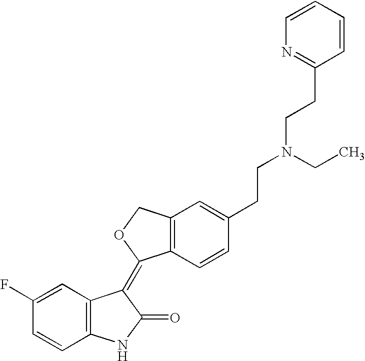 | 3-(5-{2-[Ethyl-(2-pyridin-2-yl- ethyl)-amino]-ethyl}-3H- isobenzofuran-1-ylidene)-5- fluoro-1,3-dihydro-indol-2-one | 443.519 |
| 354 | 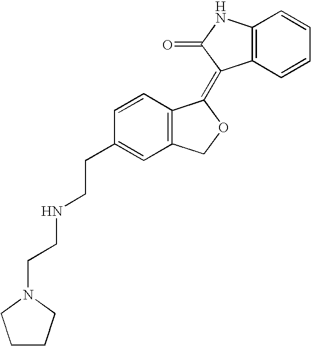 | 3-{5-[2-(2-Pyrrolidin-1-yl- ethylamino)-ethyl]-3H- isobenzofuran-1-ylidene}-1,3- dihydro-indol-2-one | 389.496 |
| 355 | 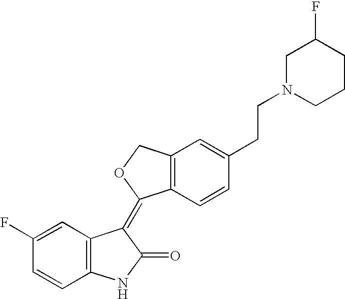 | 5-Fluoro-3-{5-[2-(3-fluoro- piperidin-1-yl)-ethyl]-3H- isobenzofuran-1-ylidene}-1,3- dihydro-indol-2-one | 396.435 |
| 356 | 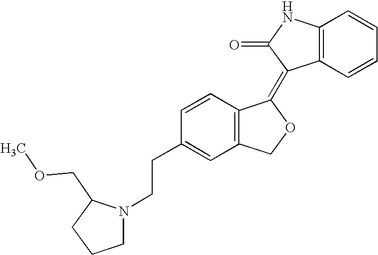 | 3-{5-[2-(2-Methoxymethyl- pyrrolidin-1-yl)-ethyl]-3H- isobenzofuran-1-ylidene}-1,3- dihydro-indol-2-one | 390.48 |
| 357 | 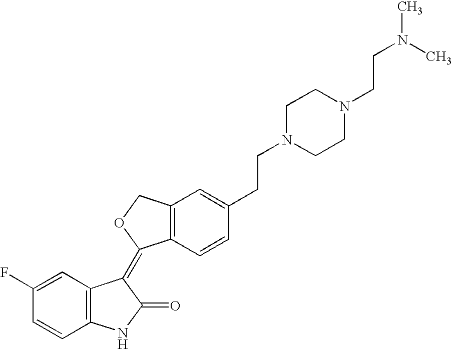 | 3-(5-{2-[4-(2-Dimethylamino- ethyl)-piperazin-1-yl]-ethyl}- 3H-isobenzofuran-1-ylidene)- 5-fluoro-1,3-dihydro-indol-2- one | 450.555 |
| 358 | 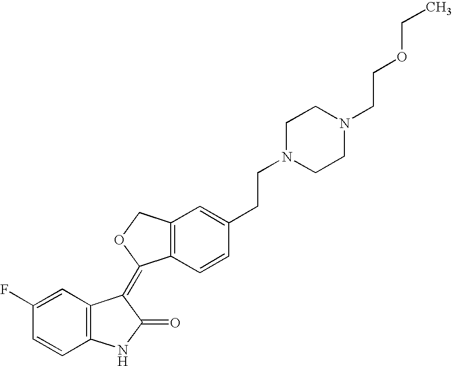 | 3-(5-{2-[4-(2-Ethoxy-ethyl)- piperazin-1-yl]-ethyl}-3H- isobenzofuran-1-ylidene)-5- fluoro-1,3-dihydro-indol-2-one | 451.539 |
| 359 | 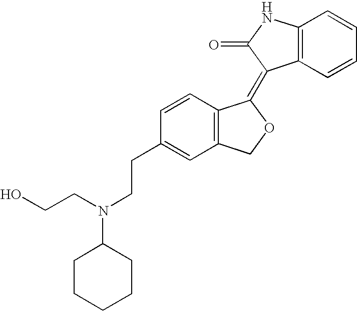 | 3-(5-{2-[Cyclohexyl-(2- hydroxy-ethyl)-amino]-ethyl}- 3H-isobenzofuran-1-ylidene)- 1,3-dihydro-indol-2-one | 418.534 |
| 360 | 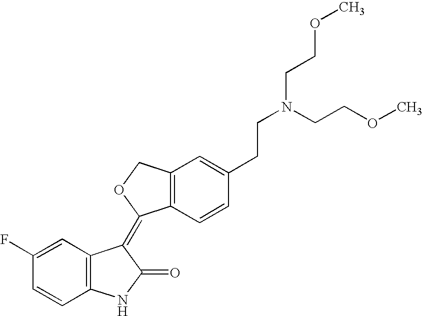 | 3-(5-{2-[Bis-(2-methoxy-ethyl)- amino]-ethyl}-3H- isobenzofuran-1-ylidene)-5- fluoro-1,3-dihydro-indol-2-one | 426.485 |
| 361 | 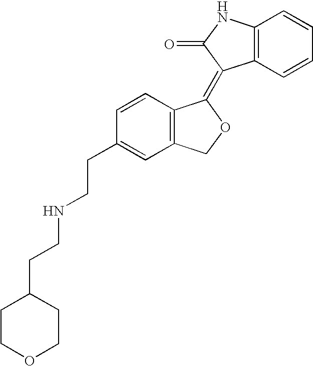 | 3-(5-{2-[2-(Tetrahydro-pyran- 4-yl)-ethylamino]-ethyl}-3H- isobenzofuran-1-ylidene)-1,3- dihydro-indol-2-one | 404.507 |
| 362 | 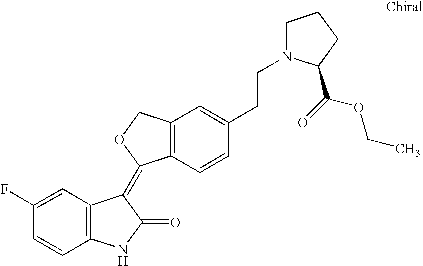 | 1-{2-[1-(5-Fluoro-2-oxo-1,2- dihydro-indol-3-ylidene)-1,3- dihydro-isobenzofuran-5-yl]- ethyl}-pyrrolidine-(S)-2- carboxylic acid ethyl ester | 436.48 |
| 363 | 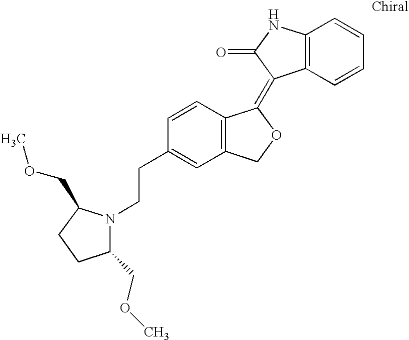 | 3-{5-[2-(S,S)-2,5-Bis- methoxymethyl-pyrrolidin-1- yl)-ethyl]-3H-isobenzofuran-1- ylidene}-1,3-dihydro-indol-2- one | 434.533 |
| 364 | 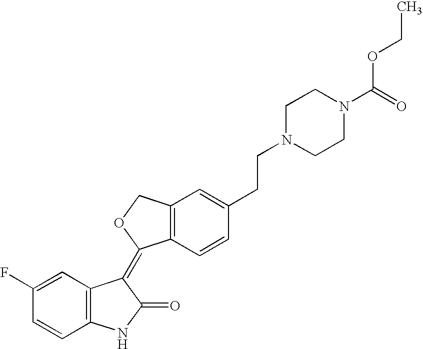 | 4-{2-[1-(5-Fluoro-2-oxo-1,2- dihydro-indol-3-ylidene)-1,3- dihydro-isobenzofuran-5-yl]- ethyl}-piperazine-1-carboxylic acid ethyl ester | 451.495 |
| 365 | 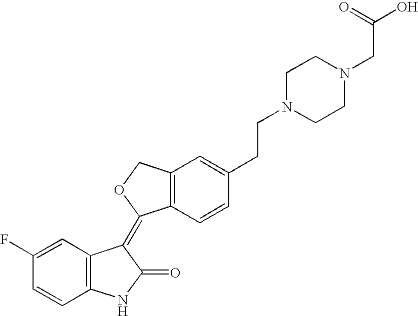 | (4-{2-[1-(5-Fluoro-2-oxo-1,2- dihydro-indol-3-ylidene)-1,3- dihydro-isobenzofuran-5-yl]- ethyl}-piperazin-1-yl)-acetic acid | 437.469 |
| 366 | 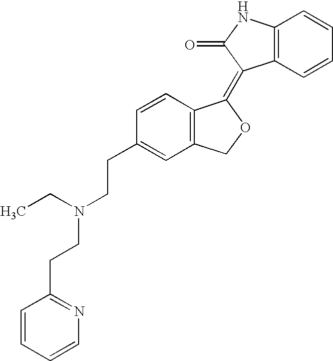 | 3-(5-{2-[Ethyl-(2-pyridin-2-yl- ethyl)-amino]-ethyl}-3H- isobenzofuran-1-ylidene)-1,3- dihydro-indol-2-one | 425.529 |
| 367 | 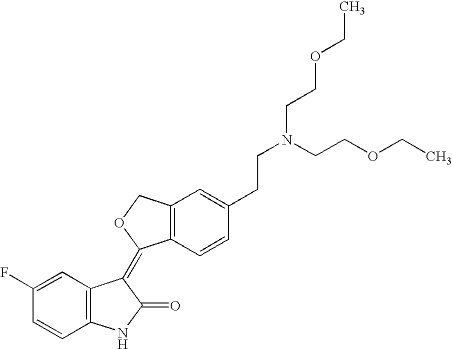 | 3-(5-{2-[Bis-(2-ethoxy-ethyl)- amino]-ethyl}-3H- isobenzofuran-1-ylidene)-5- fluoro-1,3-dihydro-indol-2-one | 454.539 |
| 368 | 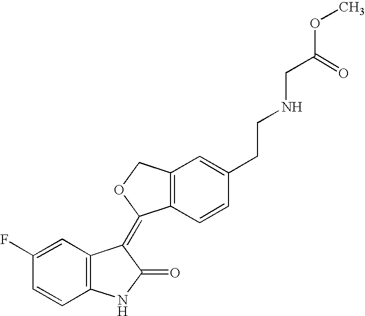 | {2-[1-(5-Fluoro-2-oxo-1,2- dihydro-indol-3-ylidene)-1,3- dihydro-isobenzofuran-5-yl]- ethylamino}-acetic acid methyl ester | 382.389 |
| 369 | 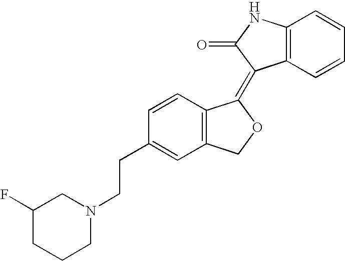 | 3-{5-[2-(3-Fluoro-piperidin-1- yl)-ethyl]-3H-isobenzofuran-1- ylidene}-1,3-dihydro-indol-2- one | 378.445 |
| 370 | 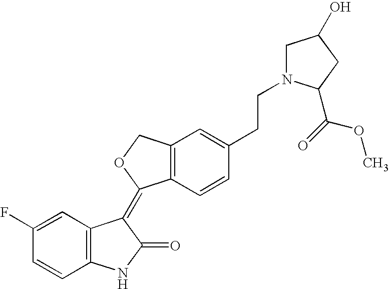 | 1-{2-[1-(5-Fluoro-2-oxo-1,2- dihydro-indol-3-ylidene)-1,3- dihydro-isobenzofuran-5-yl]- ethyl}-4-hydroxy-pyrrolidine-2- carboxylic acid methyl ester | 438.453 |
| 371 | 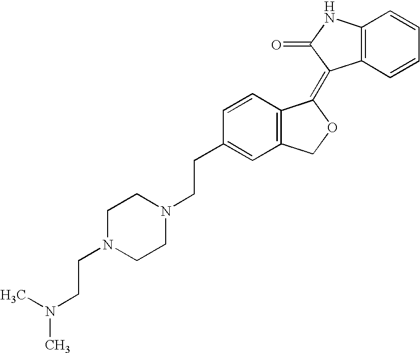 | 3-(5-{2-[4-(2-Dimethylamino- ethyl)-piperazin-1-yl]-ethyl}- 3H-isobenzofuran-1-ylidene)- 1,3-dihydro-indol-2-one | 432.565 |
| 372 | 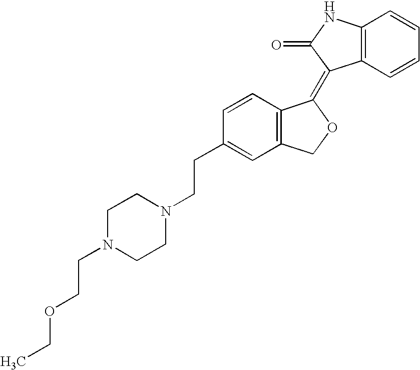 | 3-(5-{2-[4-(2-Ethoxy-ethyl)- piperazin-1-yl]-ethyl}-3H- isobenzofuran-1-ylidene)-1,3- dihydro-indol-2-one | 433.549 |
| 373 | 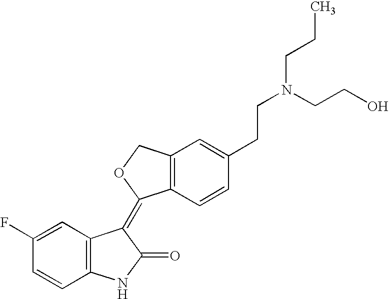 | 5-Fluoro-3-(5-{2-[(2-hydroxy- ethyl)-propyl-amino]-ethyl}- 3H-isobenzofuran-1-ylidene)- 1,3-dihydro-indol-2-one | 396.46 |
| 374 | 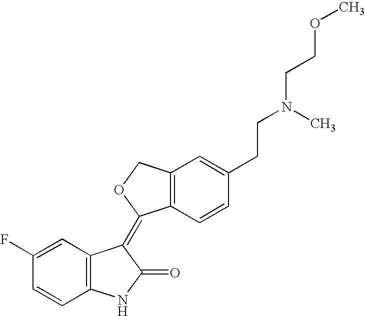 | 5-Fluoro-3-(5-{2-[(2-methoxy- ethyl)-methyl-amino]-ethyl}- 3H-isobenzofuran-1-ylidene)- 1,3-dihydro-indol-2-one | 382.433 |
| 375 | 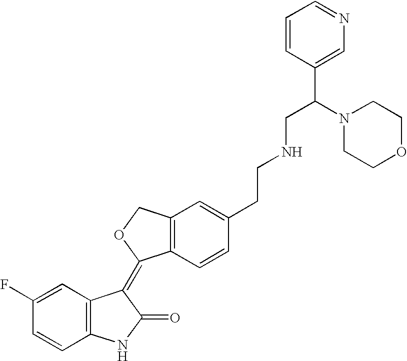 | 5-Fluoro-3-{5-[2-(2-morpholin- 4-yl-2-pyridin-3-yl-ethylamino)- ethyl]-3H-isobenzofuran-1- ylidene}-1,3-dihydro-indol-2- one | 500.571 |
| 376 | 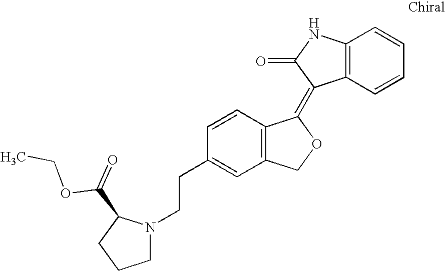 | 1-{2-[1-(2-Oxo-1,2-dihydro- indol-3-ylidene)-1,3-dihydro- isobenzofuran-5-yl]-ethyl}- pyrrolidine-(R)-2-carboxylic acid ethyl ester | 418.49 |
| 377 | 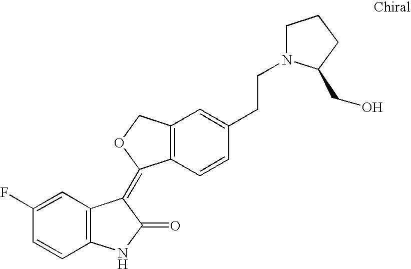 | 5-Fluoro-3-(5-{2-[(S)-2- hydroxymethyl-pyrrolidin-1-yl9 - ethyl}-3H-isobenzofuran-1- ylidene)-1,3-dihydro-indol-2- one | 394.444 |
| 378 | 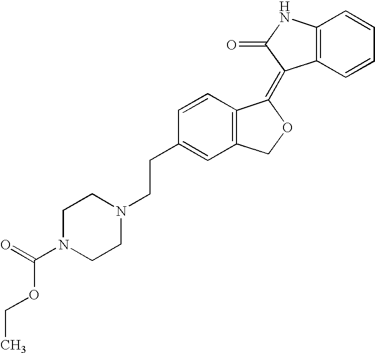 | 4-{2-[1-(2-Oxo-1,2-dihydro- indol-3-ylidene)-1,3-dihydro- isobenzofuran-5-yl]-ethyl}- piperazine-1-carboxylic acid ethyl ester | 433.505 |
| 379 | 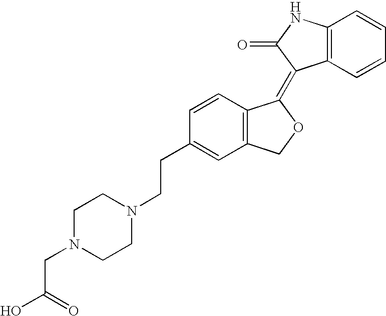 | (4-{2-[1-(2-Oxo-1,2-dihydro- indol-3-ylidene)-1,3-dihydro- isobenzofuran-5-yl]-ethyl}- piperazin-1-yl)-acetic acid | 419.478 |
| 380 | 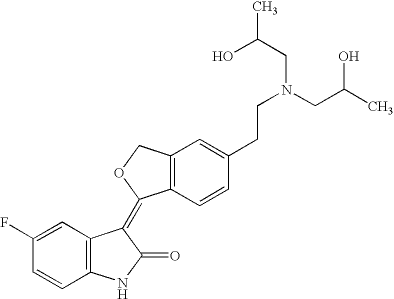 | 3-(5-{2-[Bis-(2-hydroxy- propyl)-amino]-ethyl}-3H- isobenzofuran-1-ylidene)-5- fluoro-1,3-dihydro-indol-2-one | 426.485 |
| 381 | 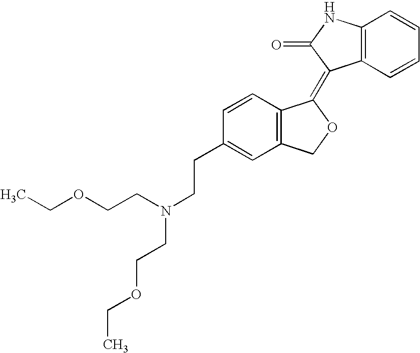 | 3-(5-{2-[Bis-(2-ethoxy-ethyl)- amino]-ethyl}-3H- isobenzofuran-1-ylidene)-1,3- dihydro-indol-2-one | 436.549 |
| 382 | 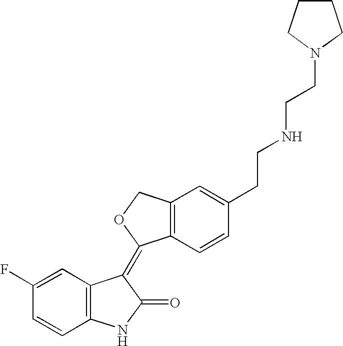 | 5-Fluoro-3-{5-[2-(2-pyrrolidin- 1-yl-ethylamino)-ethyl]-3H- isobenzofuran-1-ylidene}-1,3- dihydro-indol-2-one | 407.486 |
| 383 | 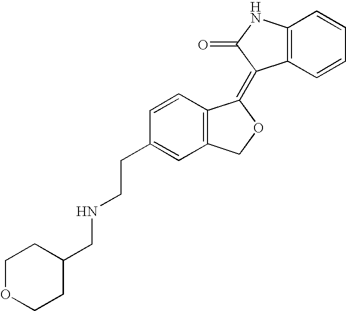 | 3-(5-{2-[(Tetrahydro-pyran-4- ylmethyl)-amino]-ethyl}-3H- isobenzofuran-1-ylidene)-1,3- dihydro-indol-2-one | 390.48 |
| 384 | 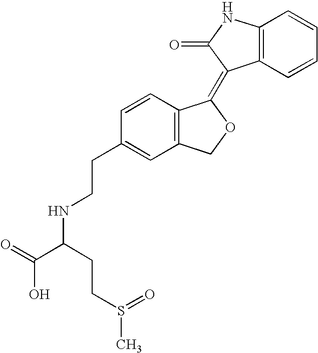 | 4-Methanesulfinyl-2-{2-[1-(2- oxo-1,2-dihydro-indol-3- ylidene)-1,3-dihydro- isobenzofuran-5-yl]- ethylamino}-butyric acid | 440.518 |
| 385 | 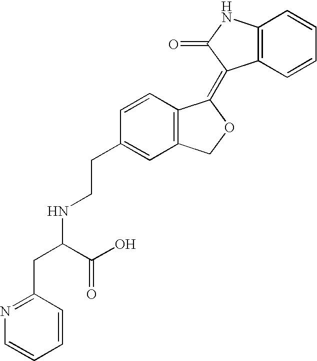 | 2-{2-[1-(2-Oxo-1,2-dihydro- indol-3-ylidene)-1,3-dihydro- isobenzofuran-5-yl]- ethylamino}-3-pyridin-2-yl- propionic acid | 441.485 |
| 386 | 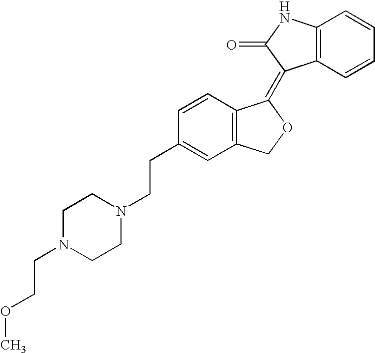 | 3-(5-{2-[4-(2-Methoxy-ethyl)- piperazin-1-yl]-ethyl}-3H- isobenzofuran-1-ylidene)-1,3- dihydro-indol-2-one | 419.522 |
| 387 | 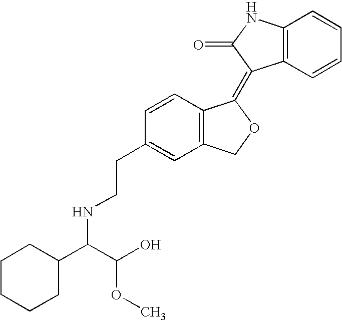 | {2-[1-(2-Oxo-1,2-dihydro- indol-3-ylidene)-1,3-dihydro- isobenzofuran-5-yl]- ethylamino}-(tetrahydro-pyran- 4-yl)-acetic acid methyl ester | 448.516 |
| 388 | 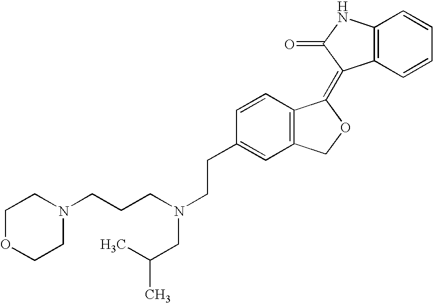 | 3-(5-{2-[Isobutyl-(3-morpholin- 4-yl-propyl)-amino]-ethyl}-3H- isobenzofuran-1-ylidene)-1,3- dihydro-indol-2-one | 475.629 |
| 389 | 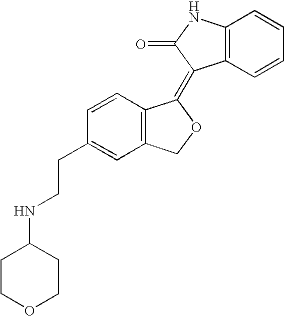 | 3-{5-[2-(Tetrahydro-pyran-4- ylamino)-ethyl]-3H- isobenzofuran-1-ylidene}-1,3- dihydro-indol-2-one | 376.454 |
| 390 | 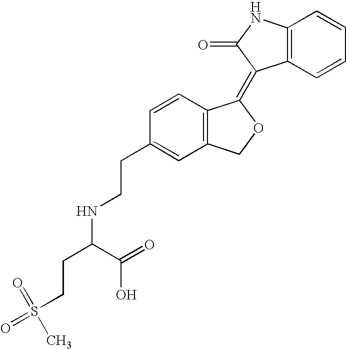 | 4-Methanesulfonyl-2-{2-[1-(2- oxo-1,2-dihydro-indol-3- ylidene)-1,3-dihydro- isobenzofuran-5-yl]- ethylamino}-butyric acid | 456.517 |
| 391 | 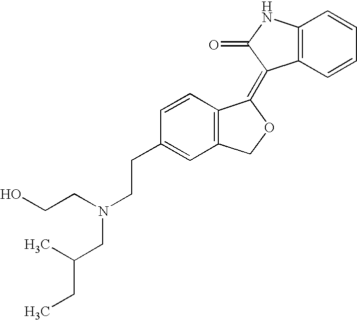 | 3-(5-{2-[(2-Hydroxy-ethyl)-(2- methyl-butyl)-amino]-ethyl}- 3H-isobenzofuran-1-ylidene)- 1,3-dihydro-indol-2-one | 406.523 |
| 392 | 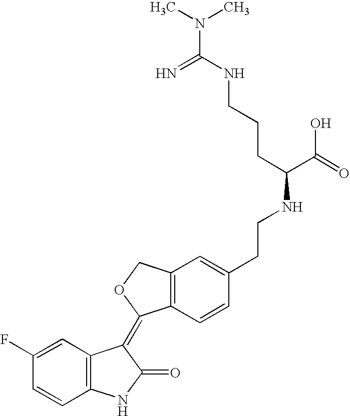 | 5-(N′,N′-Dimethyl-guanidino)- (S)-2-{2-[1-(5-fluoro-2-oxo- 1,2-dihydro-indol-3-ylidene)- 1,3-dihydro-isobenzofuran-5- yl]-ethylamino}-pentanoic acid | 495.552 |
| 393 | 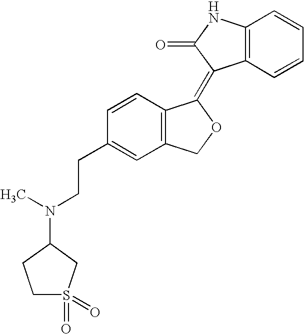 | 3-(5-{2-[(1,1-Dioxo-tetrahydro- 1lambda*6*-thiophen-3-yl)- methyl-amino]-ethyl}-3H- isobenzofuran-1-ylidene)-1,3- dihydro-indol-2-one | 424.519 |
| 394 | 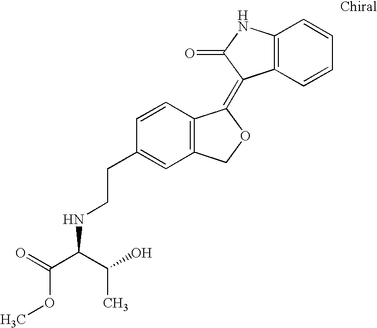 | (S)-3-Hydroxy-(S)-2-{2-[1-(2- oxo-1,2-dihydro-indol-3- ylidene)-1,3-dihydro- isobenzofuran-5-yl]- ethylaminol-butyric acid methyl ester | 408.452 |
| 395 | 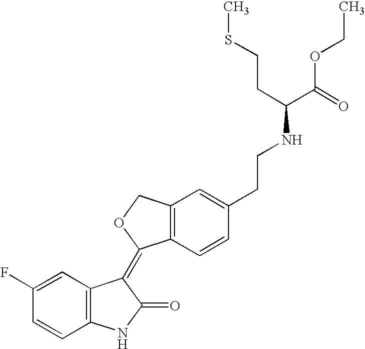 | 2-{(S)-2-[1-(5-Fluoro-2-oxo- 1,2-dihydro-indol-3-ylidene)- 1,3-dihydro-isobenzofuran-5- yl]-ethylamino}-4- methylsulfanyl-butyric acid ethyl ester | 470.562 |
| 396 | 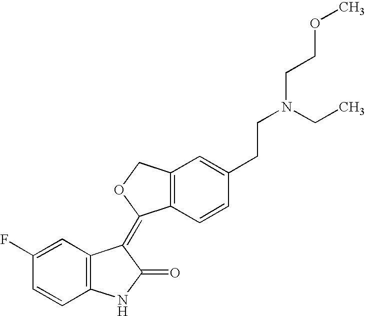 | 3-(5-{2-[Ethyl-(2-methoxy- ethyl)-amino]-ethyl}-3H- isobenzofuran-1-ylidene)-5- fluoro-1,3-dihydro-indol-2-one | 396.46 |
| 397 | 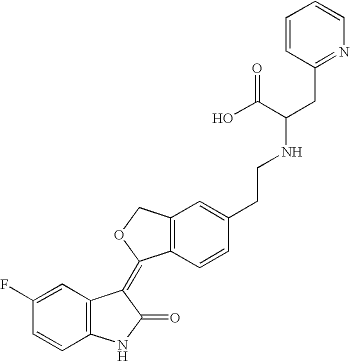 | 2-{2-[1-(5-Fluoro-2-oxo-1,2- dihydro-indol-3-ylidene)-1,3- dihydro-isobenzofuran-5-yl]- ethylamino}-3-pyridin-2-yl- propionic acid | 459.475 |
| 398 | 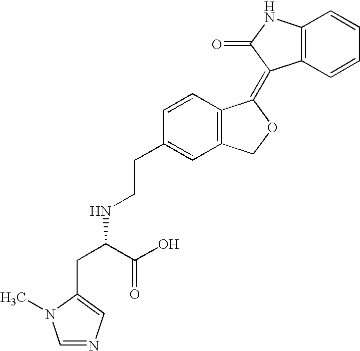 | 3-(3-Methyl-3H-imidazol-4-yl)- (S)-2-{2-[1-(2-oxo-1,2-dihydro- indol-3-ylidene)-1,3-dihydro- isobenzofuran-5-yl]- ethylamino}-propionic acid | 444.489 |
| 399 | 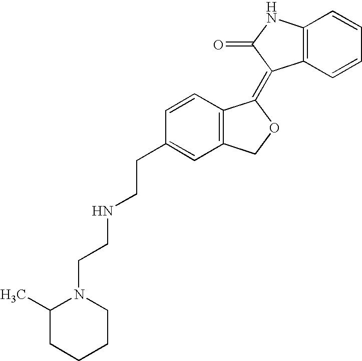 | 3-(5-{2-[2-(2-Methyl-piperidin- 1-yl)-ethylamino]-ethyl}-3H- isobenzofuran-1-ylidene)-1,3- dihydro-indol-2-one | 417.55 |
| 400 | 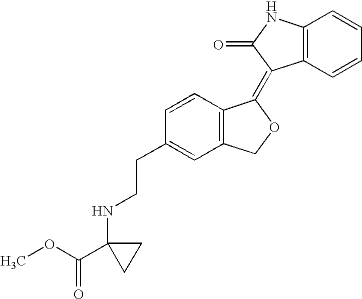 | 1-{2-[1-(2-Oxo-1,2-dihydro- indol-3-ylidene)-1,3-dihydro- isobenzofuran-5-yl]- ethylamino}- cyclopropanecarboxylic acid methyl ester | 390.437 |
| 401 | 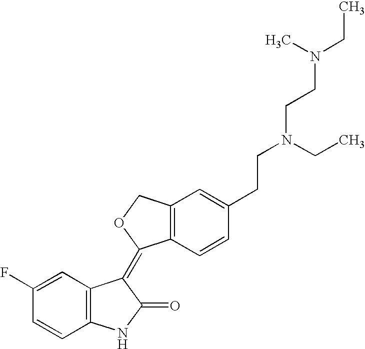 | 3-[5-(2-{Ethyl-[2-(ethyl-methyl- amino)-ethyl]-amino}-ethyl)- 3H-isobenzofuran-1-ylidene]- 5-fluoro-1,3-dihydro-indol-2- one | 423.529 |
| 402 | 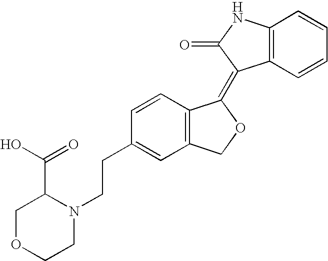 | 4-{2-[1-(2-Oxo-1,2-dihydro- indol-3-ylidene)-1,3-dihydro- isobenzofuran-5-yl]-ethyl}- morpholine-3-carboxylic acid | 406.436 |
| 403 | 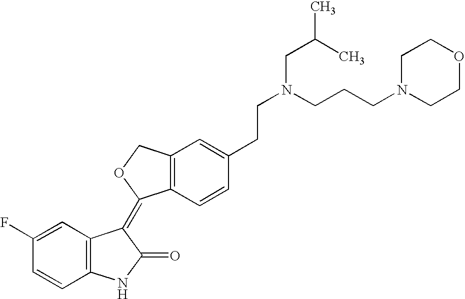 | 5-Fluoro-3-(5-{2-[isobutyl-(3- morpholin-4-yl-propyl)-amino]- ethyl}-3H-isobenzofuran-1- ylidene)-1,3-dihydro-indol-2- one | 493.619 |
| 404 | 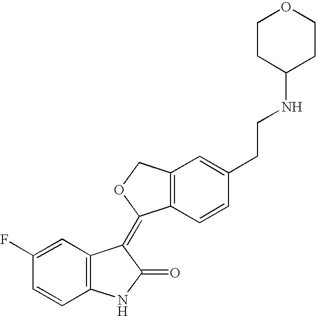 | 5-Fluoro-3-{5-[2-(tetrahydro- pyran-4-ylamino)-ethyl]-3H- isobenzofuran-1-ylidene}-1,3- dihydro-indol-2-one | 394.444 |
| 405 | 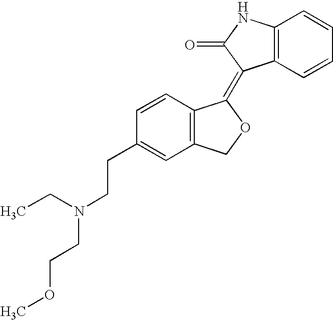 | 3-(5-{2-[Ethyl-(2-methoxy- ethyl)-amino]-ethyl}-3H- isobenzofuran-1-ylidene)-1,3- dihydro-indol-2-one | 378.469 |
| 406 | 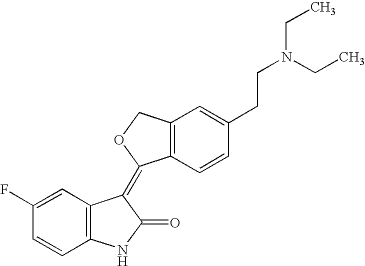 | 3-[5-(2-Diethylamino-ethyl)- 3H-isobenzofuran-1-ylidene]- 5-fluoro-1,3-dihydro-indol-2- one | 366.434 |
| 407 | 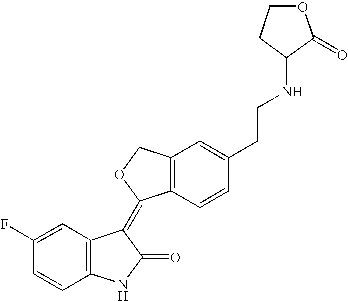 | 5-Fluoro-3-{5-[2-(2-oxo- tetrahydro-furan-3-ylamino)- ethyl]-3H-isobenzofuran-1- ylidene}-1,3-dihydro-indol- 2-one | 394.4 |
| 408 | 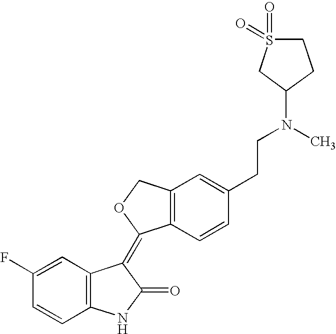 | 3-(5-{2-[(1,1-Dioxo-tetrahydro- 1lambda*6*-thiophen-3-yl)- methyl-amino]-ethyl}-3H- isobenzofuran-1-ylidene)-5- fluoro-1,3-dihydro-indol-2-one | 442.509 |
| 409 | 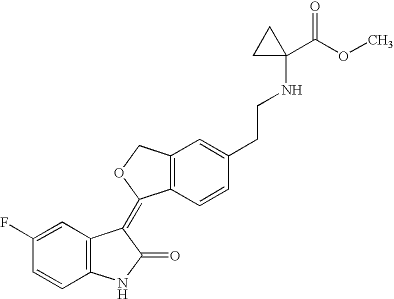 | 1-{2-[1-(5-Fluoro-2-oxo-1,2- dihydro-indol-3-ylidene)-1,3- dihydro-isobenzofuran-5-yl]- ethylamino}- cyclopropanecarboxylic acid methyl ester | 408.427 |
| 410 | 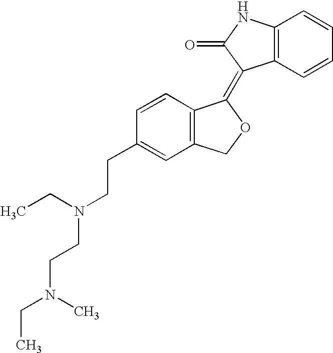 | 3-[5-(2-{Ethyl-[2-(ethyl-methyl- amino)-ethyl]-amino}-ethyl)- 3H-isobenzofuran-1-ylidene]- 1,3-dihydro-indol-2-one | 405.539 |
| 411 | 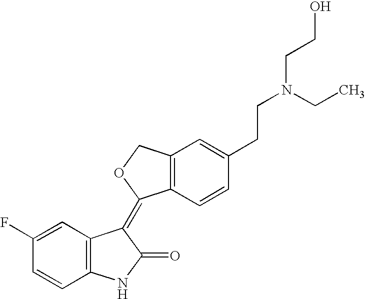 | 3-(5-{2-[Ethyl-(2-hydroxy- ethyl)-amino]-ethyl}-3H- isobenzofuran-1-ylidene)-5- fluoro-1,3-dihydro-indol-2-one | 382.433 |
| 412 | 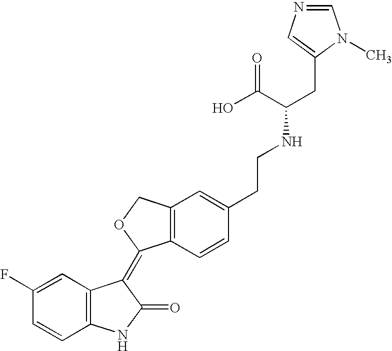 | (S)-2-{2-[1-(5-Fluoro-2-oxo- 1,2-dihydro-indol-3-ylidene)- 1,3-dihydro-isobenzofuran-5- yl]-ethylamino}-3-(3-methyl- 3H-imidazol-4-yl)-propionic acid | 462.479 |
| 413 | 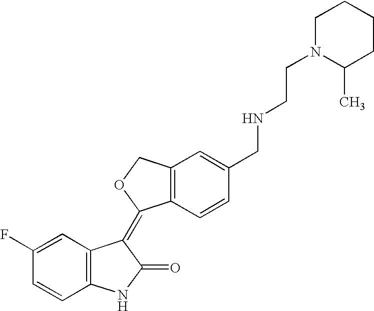 | 5-Fluoro-3-(5-{[2-(2-methyl- piperidin-1-yl)-ethylamino]- methyl}-3H-isobenzofuran-1- ylidene)-1,3-dihydro-indol-2- one | 421.513 |
| 414 | 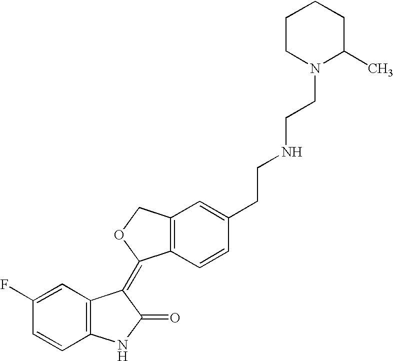 | 5-Fluoro-3-(5-{2-[2-(2-methyl- piperidin-1-yl)-ethylamino]- ethyl}-3H-isobenzofuran-1- ylidene)-1,3-dihydro-indol-2- one | 435.54 |
| 415 | 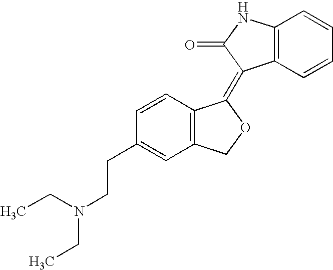 | 3-[5-(2-Diethylamino-ethyl)- 3H-isobenzofuran-1-ylidene]- 1,3-dihydro-indol-2-one | 348.444 |
| 416 | 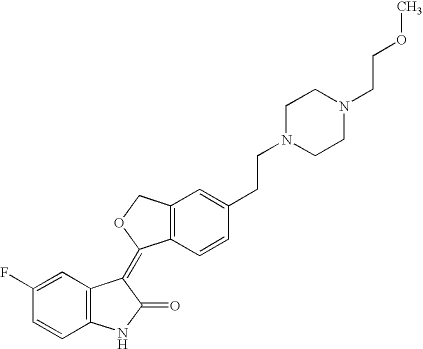 | 5-Fluoro-3-(5-{2-[4-(2- methoxy-ethyl)-piperazin-1-yl]- ethyl}-3H-isobenzofuran-1- ylidene)-1,3-dihydro-indol- 2-one | 437.512 |
| 417 | 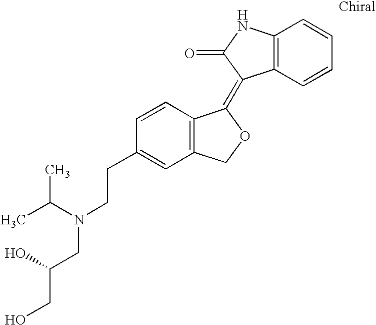 | 3-[5-(2-{[(2S)-2,3-Dihydroxy- propyl]-isopropyl-amino}- ethyl)-3H-isobenzofuran-1- ylidene]-1,3-dihydro-indol- 2-one | 408.495 |
| 418 | 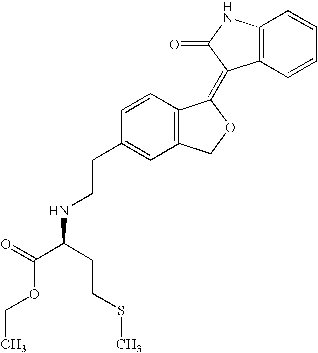 | 4-Methylsulfanyl-(S)-2-{2-[1- (2-oxo-1,2-dihydro-indol-3- ylidene)-1,3-dihydro- isobenzofuran-5-yl]- ethylaminol-butyric acid ethyl ester | 452.572 |
| 419 | 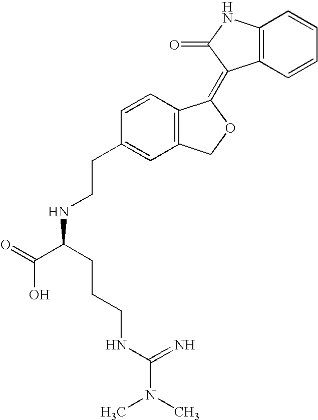 | 5-(N′,N′-Dimethyl-guanidino)- (S)-2-{2-[1-(2-oxo-1,2-dihydro- indol-3-ylidene)-1,3-dihydro- isobenzofuran-5-yl]- ethylaminol-pentanoic acid | 477.562 |
| 420 | 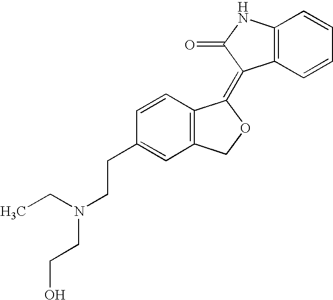 | 3-(5-{2-[Ethyl-(2-hydroxy- ethyl)-amino]-ethyl}-3H- isobenzofuran-1-ylidene)-1,3- dihydro-indol-2-one | 364.443 |
| 421 | 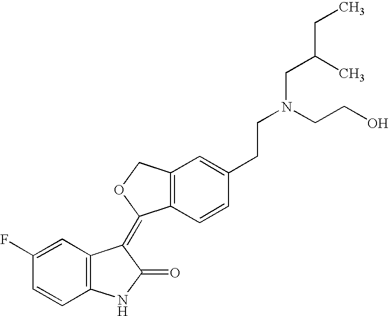 | 5-Fluoro-3-(5-{2-[(2-hydroxy- ethyl)-(2-methyl-butyl)-amino]- ethyl]-3H-isobenzofuran-1- ylidene)-1,3-dihydro-indol- 2-one | 424.513 |
| 422 | 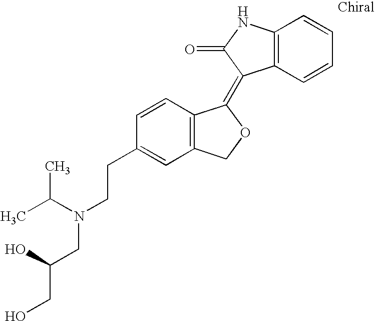 | 3-[5-(2-{[(2R)-2,3-Dihydroxy- propyl]-isopropyl-amino}- ethyl)-3H-isobenzofuran-1- ylidene]-1,3-dihydro-indol-2- one | 408.495 |
| 423 | 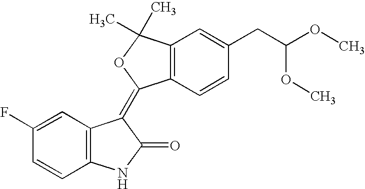 | 3-[5-(2,2-Dimethoxy-ethyl)- 3,3-dimethyl-3H- isobenzofuran-1-ylidene]-5- fluoro-1,3-dihydro-indol-2-one | 383.417 |
| 424 | 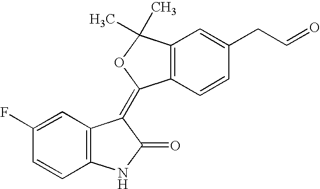 | [1-(5-Fluoro-2-oxo-1,2- dihydro-indol-3-ylidene)-3,3- dimethyl-1,3-dihydro- isobenzofuran-5-yl]- acetaldehyde | 337.348 |
| 425 | 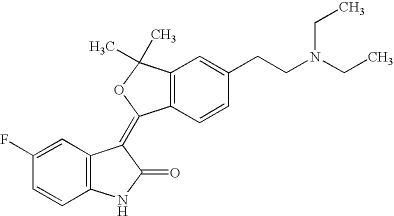 | 3-[5-(2-Diethylamino-ethyl)- 3,3-dimethyl-3H- isobenzofuran-1-ylidene]-5- fluoro-1,3-dihydro-indol-2-one | 394.487 |
| 426 | 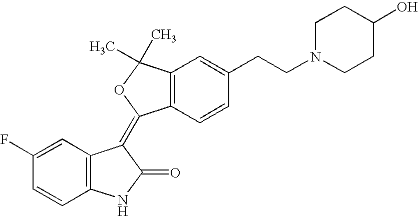 | 5-Fluoro-3-{5-[2-(4-hydroxy- piperidin-1-yl)-ethyl]-3,3- dimethyl-3H-isobenzofuran-1- ylidene}-1,3-dihydro-indol-2- one | 422.497 |
| 427 | 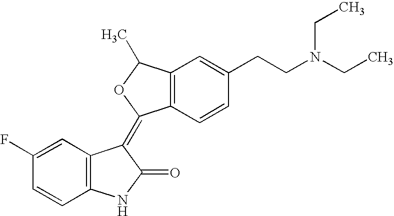 | 3-[5-(2-Diethylamino-ethyl)-3- methyl-3H-isobenzofuran-1- ylidene]-5-fluoro-1,3-dihydro- indol-2-one | 380.461 |
| 428 | 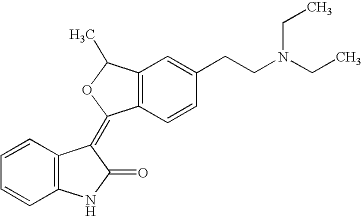 | 3-[5-(2-Diethylamino-ethyl)-3- methyl-3H-isobenzofuran-1- ylidene]-1,3-dihydro-indol-2- one | 362.47 |
| 429 | 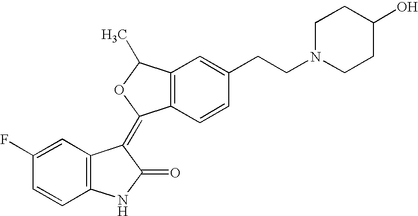 | 5-Fluoro-3-{5-[2-(4-hydroxy- piperidin-1-yl)-ethyl]-3-methyl- 3H-isobenzofuran-1-ylidene}- 1,3-dihydro-indol-2-one | 408.47 |
| 430 | 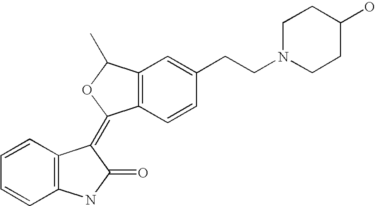 | 3-{5-[2-(4-Hydroxy-piperidin-1- yl)-ethyl]-3-methyl-3H- isobenzofuran-1-ylidene}-1,3- dihydro-indol-2-one | 390.48 |

TABLE 9

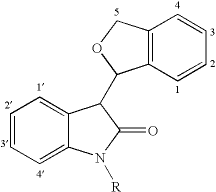

| Example | | | Molecular |
|---|---|---|---|
| Number | Chemical Structure | Chemical Name | Weight |
| 431 | 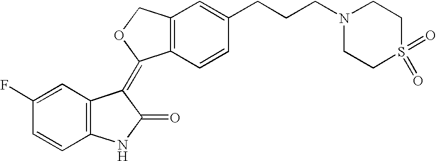 | 3-{5-[3-(1,1-Dioxo- 1lambda*6*-thiomorpholin-4- yl)-propyl]-3H-isobenzofuran- 1-ylidene}-5-fluoro-1,3- dihydro-indol-2-one | 442.509 |
| 432 | 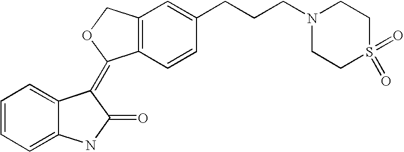 | 3-{5-[3-(1,1-Dioxo- 1lambda*6*-thiomorpholin-4- yl)-propyl]-3H-isobenzofuran- 1-ylidene}-1,3-dihydro-indol- 2-one | 424.519 |
| 433 | 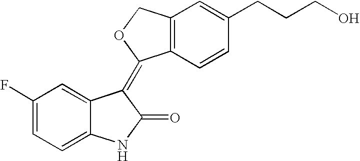 | 5-Fluoro-3-[5-(3-hydroxy- propyl)-3H-isobenzofuran-1- ylidene]-1,3-dihydro-indol-2- one | 325.337 |
| 434 | 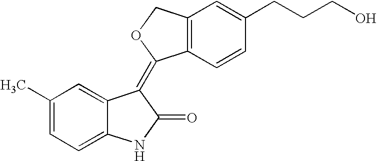 | 3-[5-(3-Hydroxy-propyl)-3H- isobenzofuran-1-ylidenel-5- methyl-1,3-dihydro-indol-2- one | 321.374 |
| 435 | 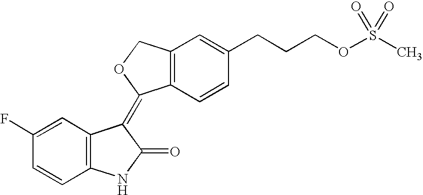 | Methanesulfonic acid 3-[1-(5- fluoro-2-oxo-1,2-dihydro-indol- 3-ylidene)-1,3-dihydro- isobenzofuran-5-yl]-propyl ester | 403.428 |
| 436 | 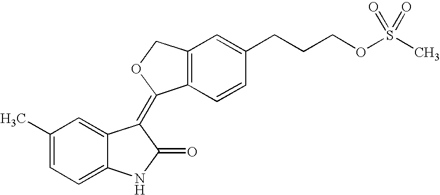 | Methanesulfonic acid 3-[1-(5- methyl-2-oxo-1,2-dihydro- indol-3-ylidene)-1,3-dihydro- isobenzofuran-5-yl]-propyl ester | 399.465 |
| 437 | 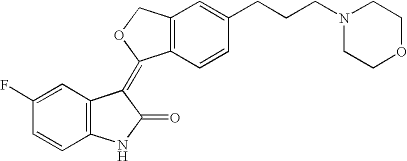 | 5-Fluoro-3-[5-(3-morpholin-4- yl-propyl)-3H-isobenzofuran- 1-ylidene]-1,3-dihydro-indol-2- one | 394.444 |
| 438 | 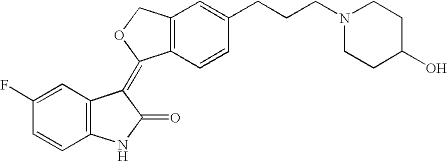 | 5-Fluoro-3-{5-[3-(4-hydroxy- piperidin-1-yl)-propyll-3H- isobenzofuran-1-ylidene}-1,3- dihydro-indol-2-one | 408.47 |
| 439 | 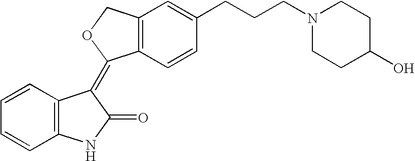 | 3-{5-[3-(4-Hydroxy-piperidin-1- yl)-propyl]-3H-isobenzofuran- 1-ylidene}-1,3-dihydro-indol- 2-one | 390.48 |
| 440 | 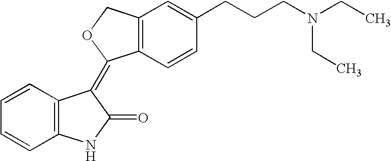 | 3-[5-(3-Diethylamino-propyl)- 3H-isobenzofuran-1-ylidene]- 1,3-dihydro-indol-2-one | 362.47 |
| 441 | 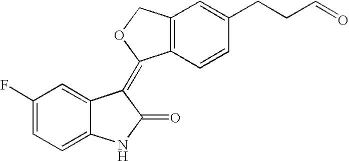 | 3-[1-(5-Fluoro-2-oxo-1,2- dihydro-indol-3-ylidene)-1- dihydro-isobenzofuran-5-yl]- propionaldehyde | 323.322 |
| 442 | 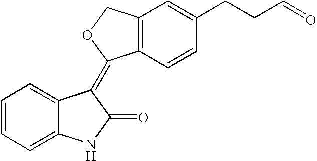 | 3-[1-(2-Oxo-1,2-dihydro-indol- 3-ylidene)-1,3-dihydro- isobenzofuran-5-yl]- propionaldehyde | 305.332 |
| 443 | 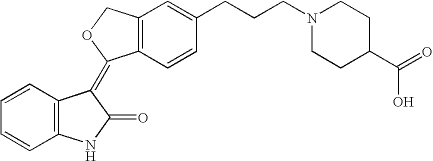 | 1-{3-[1-(2-Oxo-1,2-dihydro- indol-3-ylidene)-1,3-dihydro- isobenzofuran-5-yl]-propyl}- piperidine-4-carboxylic acid | 418.49 |
| 444 | 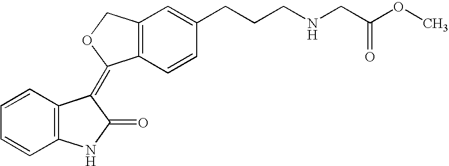 | {3-[1-(2-Oxo-1,2-dihydro- indol-3-ylidene)-1,3-dihydro- isobenzofuran-5-yl]- propylamino}-acetic acid methyl ester | 378.426 |
| 445 | 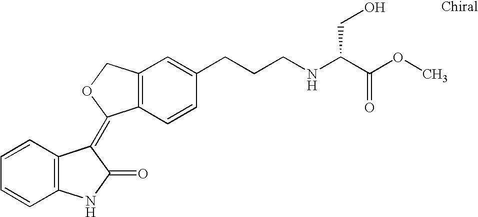 | 3-Hydroxy-2-{3-[1-(2-oxo-1,2- dihydro-indol-3-ylidene)-1,3- dihydro-isobenzofuran-5-yl]- propylamino}-propionic acid methyl ester | 408.452 |
| 446 | 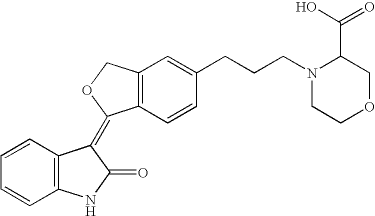 | 4-{3-[1-(2-Oxo-1,2-dihydro- indol-3-ylidene)-1,3-dihydro- isobenzofuran-5-yl]-propyl}- morpholine-3-carboxylic acid | 420.463 |
| 447 | 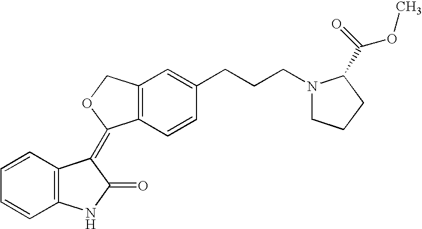 | 1-{3-[1-(2-Oxo-1,2-dihydro- indol-3-ylidene)-1,3-dihydro- isobenzofuran-5-yl]-propyl}- pyrrolidine-2-carboxylic acid methyl ester | 418.49 |
| 448 | 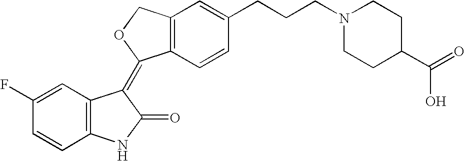 | 1-{3-[1-(5-Fluoro-2-oxo-1,2- dihydro-indol-3-ylidene)-1,3- dihydro-isobenzofuran-5-yl]- propyl}-piperidine-4- carboxylic acid | 436.48 |
| 449 | 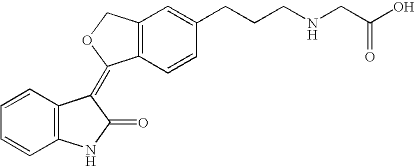 | {3-[1-(2-Oxo-1,2-dihydro- indol-3-ylidene)-1,3-dihydro- isobenzofuran-5-yl]- propylamino}-acetic acid | 364.399 |
| 450 | 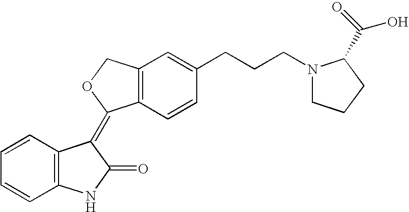 | 1-{3-[1-(2-Oxo-1,2-dihydro- indol-3-ylidene)-1,3-dihydro- isobenzofuran-5-yl]-propyl}- pyrrolidine-2-carboxylic acid | 404.464 |
| 451 | 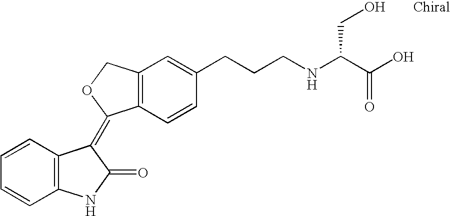 | 3-Hydroxy-2-{3-[1-(2-oxo-1,2- dihydro-indol-3-ylidene)-1,3- dihydro-isobenzofuran-5-yl]- propylamino}-propionic acid | 394.425 |
| 452 | 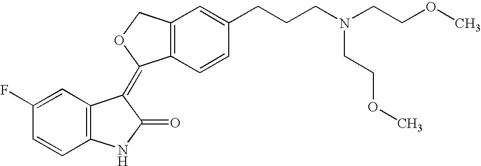 | 3-(5-{3-[Bis-(2-methoxy-ethyl)- amino]-propyl}-3H- isobenzofuran-1-ylidene)-5- fluoro-1,3-dihydro-indol-2-one | 440.512 |
| 453 | 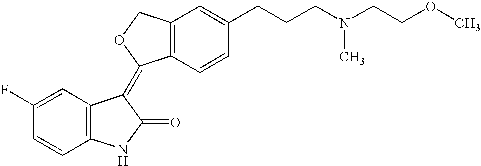 | 5-Fluoro-3-(5-{3-[(2-methoxy- ethyl)-methyl-amino]-propyl}- 3H-isobenzofuran-1-ylidene)- 1,3-dihydro-indol-2-one | 396.46 |
| 454 | 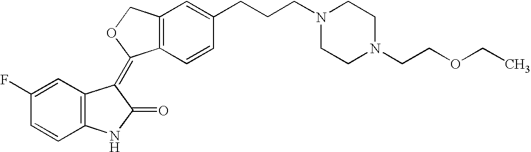 | 3-(5-{3-[4-(2-Ethoxy-ethyl)- piperazin-1-yl]-propyl}-3H- isobenzofuran-1-ylidene)-5- fluoro-1,3-dihydro-indol-2-one | 465.566 |
| 455 | 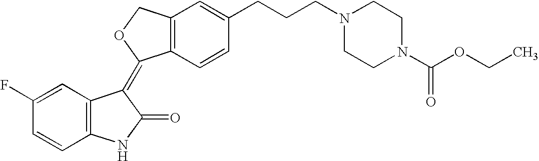 | 4-{3-[1-(5-Fluoro-2-oxo-1,2- dihydro-indol-3-ylidene)-1,3- dihydro-isobenzofuran-5-yl]- propyl}-piperazine-1- carboxylic acid ethyl ester | 465.522 |
| 456 | 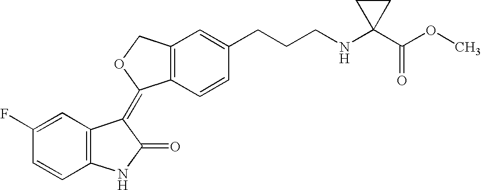 | 1-{3-[1-(5-Fluoro-2-oxo-1,2- dihydro-indol-3-ylidene)-1,3- dihydro-isobenzofuran-5-yl]- propylamino}- cyclopropanecarboxylic acid methyl ester | 422.454 |
| 457 | 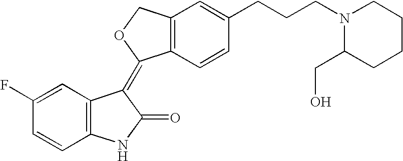 | 5-Fluoro-3-{5-[3-(2- hydroxymethyl-piperidin-1-yl)- propyl]-3H-isobenzofuran-1- ylidene}-1,3-dihydro-indol- 2-one | 422.497 |
| 458 | 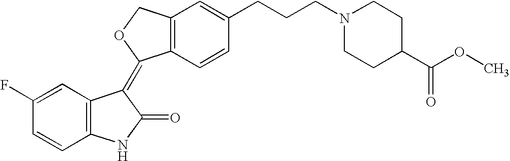 | 1-{3-[1-(5-Fluoro-2-oxo-1,2- dihydro-indol-3-ylidene)-1,3- dihydro-isobenzofuran-5-yl]- propyl}-piperidine-4-carboxylic acid methyl ester | 450.507 |
| 459 | 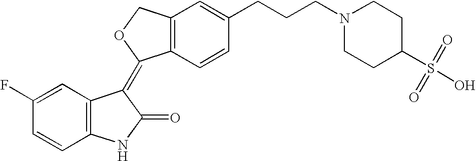 | 1-{3-[1-(5-Fluoro-2-oxo-1,2- dihydro-indol-3-ylidene)-1,3- dihydro-isobenzofuran-5-yl]- propyl}-piperidine-4-sulfonic acid | 472.534 |
| 460 | 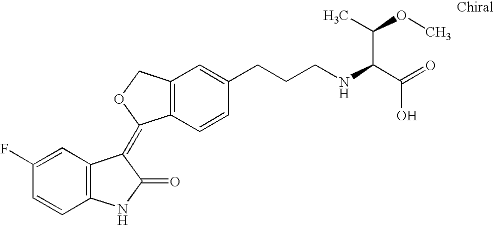 | 2-{3-[1-(5-Fluoro-2-oxo-1,2- dihydro-indol-3-ylidene)-1,3- dihydro-isobenzofuran-5-yl]- propylamino}-3-methoxy- butyric acid | 440.469 |
| 461 | 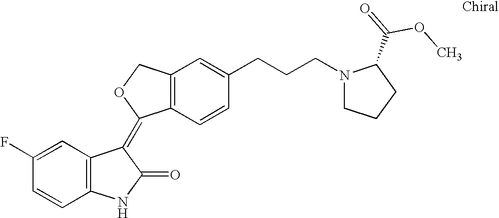 | 1-{3-[1-(5-Fluoro-2-oxo-1,2- dihydro-indol-3-ylidene)-1,3- dihydro-isobenzofuran-5-yl]- propyl}-pyrrolidine-2- carboxylic acid methyl ester | 436.48 |
| 462 | 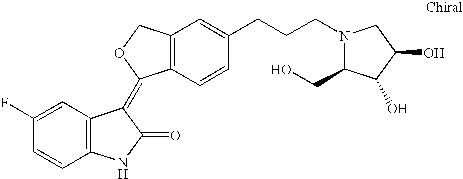 | 3-{5-[3-(3,4-Dihydroxy-2- hydroxymethyl-pyrrolidin-1-yl)- propyl]-3H-isobenzofuran-1- ylidene}-5-fluoro-1,3-dihydro- indol-2-one | 440.469 |
| 463 | 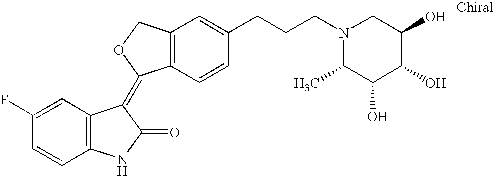 | 5-Fluoro-3-{5-[3-(3,4,5- trihydroxy-2-methyl-piperidin- 1-yl)-propyl]-3H- isobenzofuran-1-ylidene}-1,3- dihydro-indol-2-one | 454.495 |
| 464 | 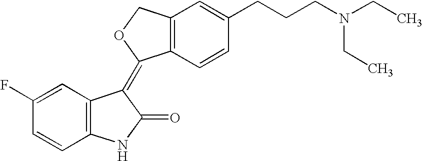 | 3-[5-(3-Diethylamino-propyl)- 3H-isobenzofuran-1-ylidene]- 5-fluoro-1,3-dihydro-indol- 2-one | 380.461 |
| 465 | 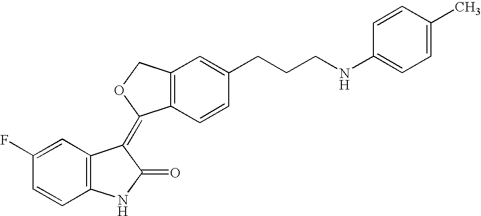 | 5-Fluoro-3-[5-(3-p-tolylamino- propyl)-3H-isobenzofuran-1- ylidene]-1,3-dihydro-indol- 2-one | 414.478 |
| 466 | 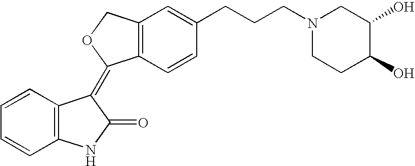 | trans-3-{5-[3-(3,4-Dihydroxy- piperidin-1-yl)-propyl]-3H- isobenzofuran-1-ylidene}-1,3- dihydro-indol-2-one | 406.479 |
| 467 | 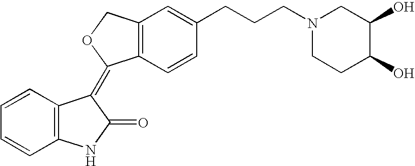 | cis-3-{5-[3-(3,4-Dihydroxy- piperidin-1-yl)-propyl]-3H- isobenzofuran-1-ylidene}-1,3- dihydro-indol-2-one | 406.479 |
| 468 | 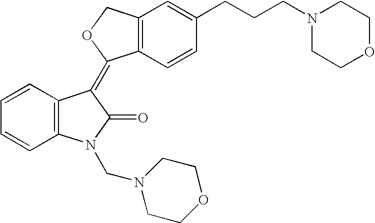 | 1-Morpholin-4-ylmethyl-3-[5- (3-morpholin-4-yl-propyl)-3H- isobenzofuran-1-ylidene]-1,3- dihydro-indol-2-one | 475.586 |
| 469 | 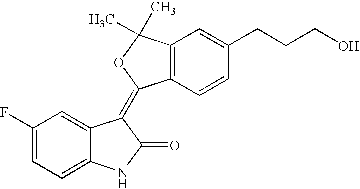 | 5-Fluoro-3-[5-(3-hydroxy- propyl)-3,3-dimethyl-3H- isobenzofuran-1-ylidene]-1,3- dihydro-indol-2-one | 353.391 |
| 470 | 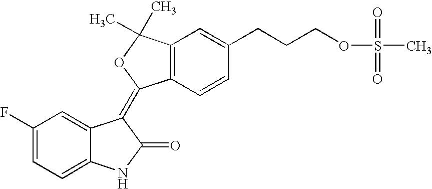 | Methanesulfonic acid 3-[1-(5- fluoro-2-oxo-1,2-dihydro-indol- 3-ylidene)-3,3-dimethyl-1,3- dihydro-isobenzofuran-5-yl]- propyl ester | 431.482 |
| 471 | 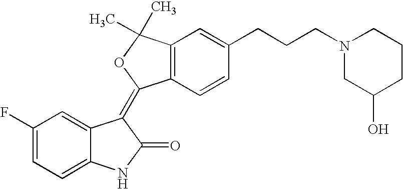 | 5-Fluoro-3-{5-[3-(3-hydroxy- piperidin-1-yl)-propyl]-3,3- dimethyl-3H-isobenzofuran-1- ylidene}-1,3-dihydro-indol- 2-one | 436.524 |
| 472 | 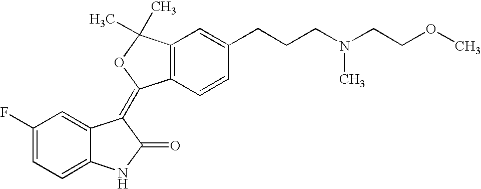 | 5-Fluoro-3-(5-{3-[(2-methoxy- ethyl)-methyl-amino]-propyl}- 3,3-dimethyl-3H- isobenzofuran-1-ylidene)-1,3- dihydro-indol-2-one | 424.513 |
| 473 | 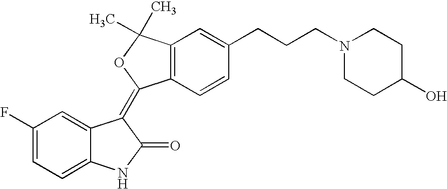 | 5-Fluoro-3-{5-[3-(4-hydroxy- piperidin-1-yl)-propyl]-3,3- dimethyl-3H-isobenzofuran-1- ylidene}-1,3-dihydro-indol- 2-one | 436.524 |
| 474 | 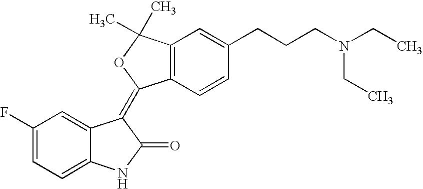 | 3-[5-(3-Diethylamino-propyl)- 3,3-dimethyl-3H- isobenzofuran-1-ylidene]-5- fluoro-1,3-dihydro-indol-2-one | 408.514 |
| 475 | 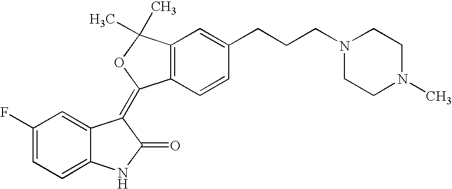 | 3-{3,3-Dimethyl-5-[3-(4- methyl-piperazin-1-yl)-propyl]- 3H-isobenzofuran-1-ylidene}- 5-fluoro-1,3-dihydro-indol- 2-one | 435.54 |
| 476 | 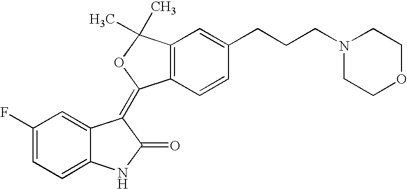 | 3-[3,3-Dimethyl-5-(3- morpholin-4-yl-propyl)-3H- isobenzofuran-1-ylidene]-5- fluoro-1,3-dihydro-indol-2-one | 422.497 |
| 477 | 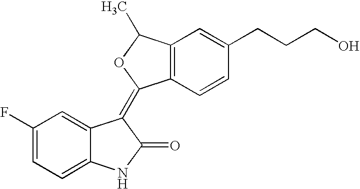 | 5-Fluoro-3-[5-(3-hydroxy- propyl)-3-methyl-3H- isobenzofuran-1-ylidene]-1,3- dihydro-indol-2-one | 339.364 |
| 478 | 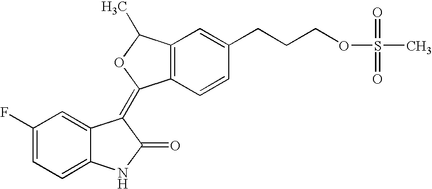 | Methanesulfonic acid 3-[1-(5- fluoro-2-oxo-1,2-dihydro-indol- 3-ylidene)-3-methyl-1,3- dihydro-isobenzofuran-5-yl]- propyl ester | 417.455 |
| 479 | 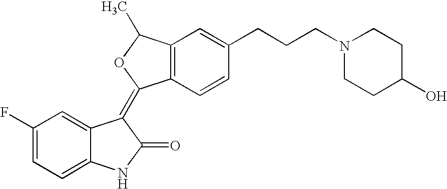 | 5-Fluoro-3-{5-[3-(4-hydroxy- piperidin-1-yl)-propyl]-3- methyl-3H-isobenzofuran-1- ylidene}-1,3-dihydro-indol- 2-one | 422.497 |
| 480 | 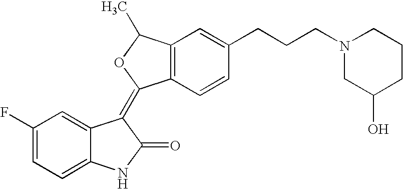 | 5-Fluoro-3-{5-[3-(3-hydroxy- piperidin-1-yl)-propyl]-3- methyl-3H-isobenzofuran-1- ylidene}-1,3-dihydro-indol- 2-one | 422.497 |
| 481 | 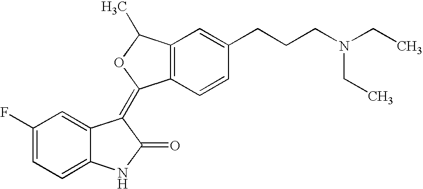 | 3-[5-(3-Diethylamino-propyl)- 3-methyl-3H-isobenzofuran-1- ylidene]-5-fluoro-1,3-dihydro- indol-2-one | 394.487 |
| 482 | 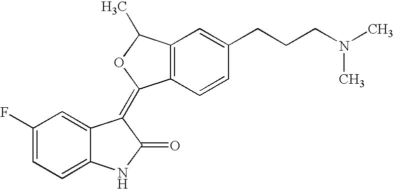 | 3-[5-(3-Dimethylamino- propyl)-3-methyl-3H- isobenzofuran-1-ylidene]-5- fluoro-1,3-dihydro-indol-2-one | 366.434 |
| 483 | 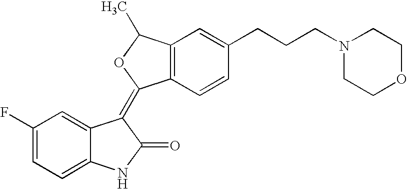 | 5-Fluoro-3-[3-methyl-5-(3- morpholin-4-yl-propyl)-3H- isobenzofuran-1-ylidene]-1,3- dihydro-indol-2-one | 408.47 |
| 484 | 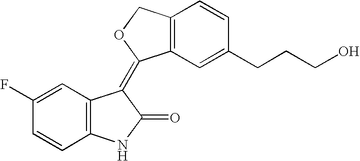 | 5-Fluoro-3-[6-(3-hydroxy- propyl)-3H-isobenzofuran-1- ylidene]-1,3-dihydro-indol- 2-one | 325.337 |

TABLE 10

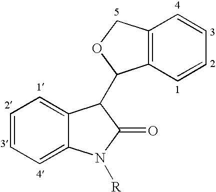

| Example | | | Molecular |
|---|---|---|---|
| Number | Chemical Structure | Chemical Name | Weight |
| 485 | 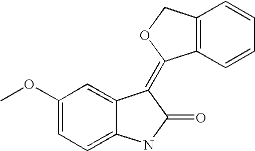 | 3-(3H-Isobenzofuran-1- ylidene)-5-methoxy-1,3- dihydro-indol-2-one | 279.294 |
| 486 | 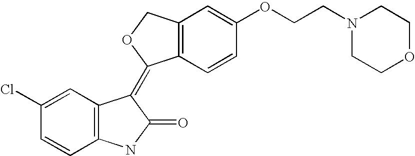 | 5-Chloro-3-[5-(2-morpholin-4- yl-ethoxy)-3H-isobenzofuran- 1-ylidene]-1,3-dihydro-indol-2- one | 412.871 |
| 487 | 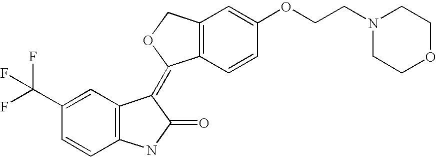 | 3-[5-(2-Morpholin-4-yl- ethoxy)-3H-isobenzofuran-1- ylidene]-5-trifluoromethyl-1,3- dihydro-indol-2-one | 446.423 |
| 488 | 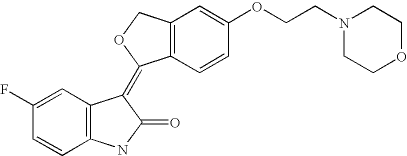 | 5-Fluoro-3-[5-(2-morpholin-4- yl-ethoxy)-3H-isobenzofuran- 1-ylidene]-1,3-dihydro-indol-2- one | 396.416 |
| 489 | 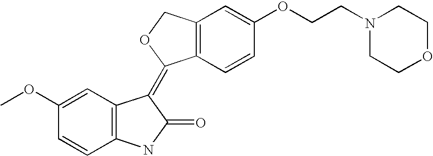 | 5-Methoxy-3-[5-(2-morpholin- 4-yl-ethoxy)-3H- isobenzofuran-1-ylidene]-1,3- dihydro-indol-2-one | 408.452 |
| 490 | 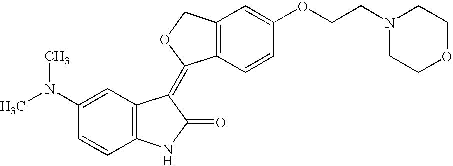 | 5-Dimethylamino-3-[5-(2- morpholin-4-yl-ethoxy)-3H- isobenzofuran-1-ylidene]-1,3- dihydro-indol-2-one | 421.494 |
| 491 | 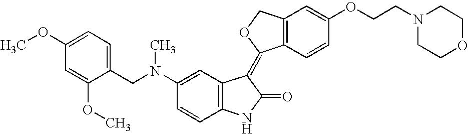 | 5-[(2,4-Dimethoxy-benzyl)- methyl-amino]-3-[5-(2- morpholin-4-yl-ethoxy)-3H- isobenzofuran-1-ylidene]-1,3- dihydro-indol-2-one | 557.643 |
| 492 | 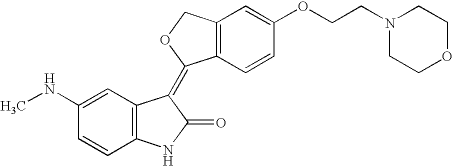 | 5-Methylamino-3-[5-(2- morpholin-4-yl-ethoxy)-3H- isobenzofuran-1-ylidene]-1,3- dihydro-indol-2-one | 407.467 |
| 493 | 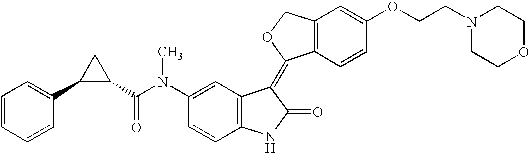 | 2-Phenyl- cyclopropanecarboxylic acid methyl-{3-[5-(2-morpholin-4- yl-ethoxy)-3H-isobenzofuran- 1-ylidene]-2-oxo-2,3-dihydro- 1H-indol-5-yl}-amide | 551.64 |
| 494 | 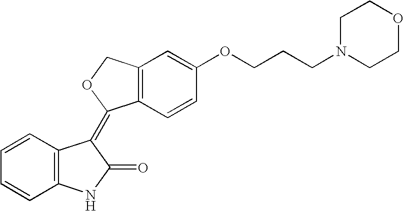 | 3-[5-(3-Morpholin-4-yl- propoxy)-3H-isobenzofuran-1- ylidene]-1,3-dihydro-indol-2- one | 392.453 |
| 495 | 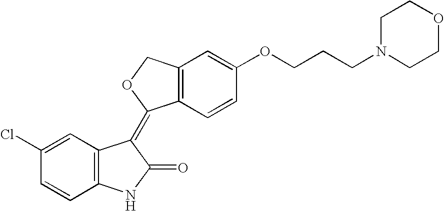 | 5-Chloro-3-[5-(3-morpholin-4- yl-propoxy)-3H-isobenzofuran- 1-ylidene]-1,3-dihydro-indol-2- one | 426.898 |
| 496 | 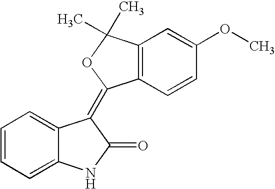 | 3-(5-Methoxy-3,3-dimethyl- 3H-isobenzofuran-1-ylidene)- 1,3-dihydro-indol-2-one | 307.347 |
| 497 | 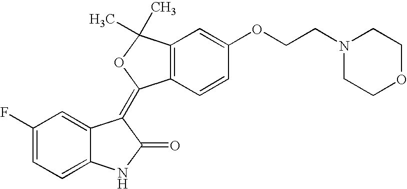 | 3-[3,3-Dimethyl-5-(2- morpholin-4-yl-ethoxy)-3H- isobenzofuran-1-ylidene]-5- fluoro-1,3-dihydro-indol-2-one | 424.47 |
| 498 | 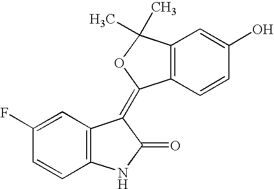 | 5-Fluoro-3-(5-hydroxy-3,3- dimethyl-3H-isobenzofuran-1- ylidene)-1,3-dihydro-indol-2- one | 311.311 |
| 499 | 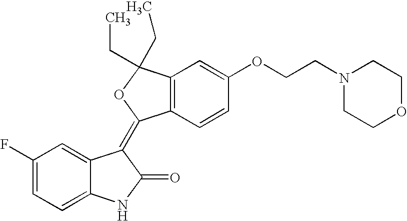 | 3-[3,3-Diethyl-5-(2-morpholin- 4-yl-ethoxy)-3H- isobenzofuran-1-ylidene]-5- fluoro-1,3-dihydro-indol-2-one | 452.523 |
| 500 | 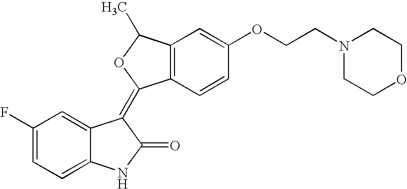 | 5-Fluoro-3-[3-methyl-5-(2- morpholin-4-yl-ethoxy)-3H- isobenzofuran-1-ylidene]-1,3- dihydro-indol-2-one | 410.443 |
| 501 | 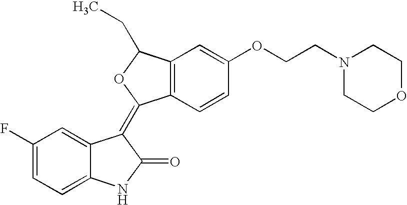 | 3-[3-Ethyl-5-(2-morpholin-4-yl- ethoxy)-3H-isobenzofuran-1- ylidene]-5-fluoro-1,3-dihydro- indol-2-one | 424.47 |
| 502 | 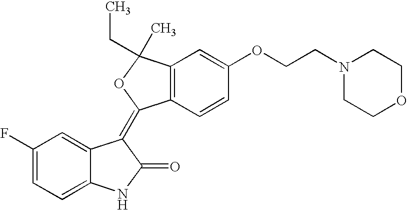 | 3-[3-Ethyl-3-methyl-5-(2- morpholin-4-yl-ethoxy)-3H- isobenzofuran-1-ylidene]-5- fluoro-1,3-dihydro-indol-2-one | 438.496 |
| 503 | 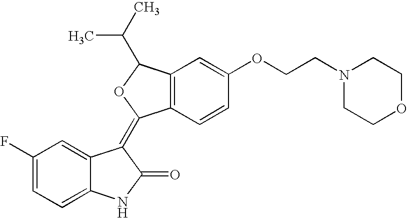 | 5-Fluoro-3-[3-isopropyl-5-(2- morpholin-4-yl-ethoxy)-3H- isobenzofuran-1-ylidene]-1,3- dihydro-indol-2-one | 438.496 |
| 504 | 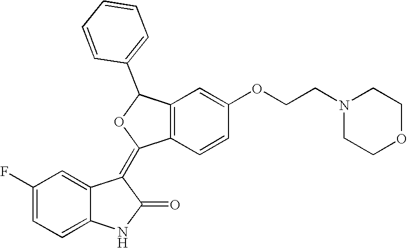 | 5-Fluoro-3-[5-(2-morpholin-4- yl-ethoxy)-3-phenyl-3H- isobenzofuran-1-ylidene]-1,3- dihydro-indol-2-one | 472.513 |
| 505 | 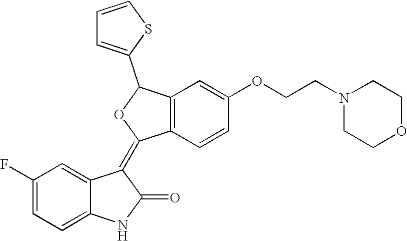 | 5-Fluoro-3-[5-(2-morpholin-4- yl-ethoxy)-3-thiophen-2-yl-3H- isobenzofuran-1-ylidene]-1,3- dihydro-indol-2-one | 478.542 |

TABLE 11

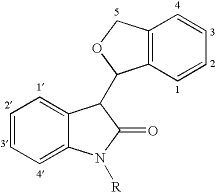

| Example | | | Molecular |
|---|---|---|---|
| Number | Chemical Structure | Chemical Name | Weight |
| 506 | 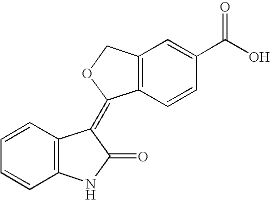 | 1-(2-Oxo-1,2-dihydro-indol-3- ylidene)-1,3-dihydro- isobenzofuran-5-carboxylic acid | 293.277 |
| 507 | 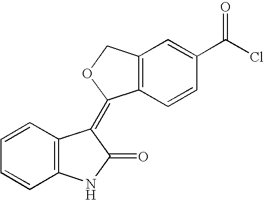 | 1-(2-Oxo-1,2-dihydro-indol-3- ylidene)-1,3-dihydro- isobenzofuran-5-carbonyl chloride | 311.723 |
| 508 | 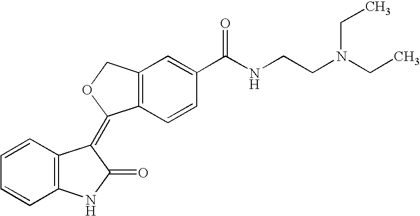 | 1-(2-Oxo-1,2-dihydro-indol-3- ylidene)-1,3-dihydro- isobenzofuran-5-carboxylic acid (2-diethylamino-ethyl)- amide | 391.469 |
| 509 | 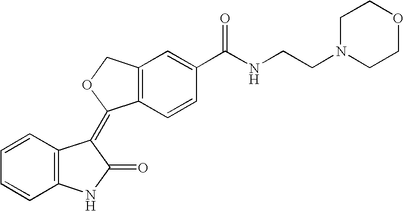 | 1-(2-Oxo-1,2-dihydro-indol-3- ylidene)-1,3-dihydro- isobenzofuran-5-carboxylic acid (2-morpholin-4-yl- ethyl)-amide | 405.452 |
| 510 | 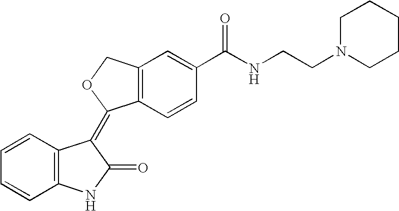 | 1-(2-Oxo-1,2-dihydro-indol-3- ylidene)-1,3-dihydro- isobenzofuran-5-carboxylic acid (2-piperidin-1-yl-ethyl)- amide | 403.479 |
| 511 | 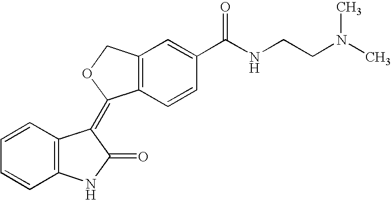 | 1-(2-Oxo-1,2-dihydro-indol-3- ylidene)-1,3-dihydro- isobenzofuran-5-carboxylic acid (2-dimethylamino- ethyl)-amide | 363.415 |
| 512 | 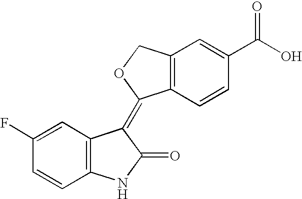 | 1-(5-Fluoro-2-oxo-1,2- dihydro-indol-3-ylidene)-1,3- dihydro-isobenzofuran-5- carboxylic acid | 311.267 |
| 513 | 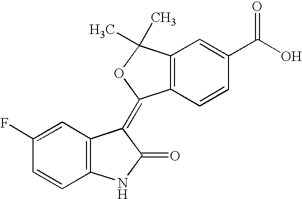 | 1-(5-Fluoro-2-oxo-1,2- dihydro-indolo-isobenzofuran- 5-carboxylic acid | 339.321 |
| 514 | 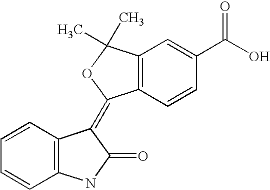 | 3,3-Dimethyl-1-(2-oxo-1,2- dihydro-indol-3-ylidene)-1,3- dihydro-isobenzofuran-5- carboxylic acid | 321.331 |
| 515 | 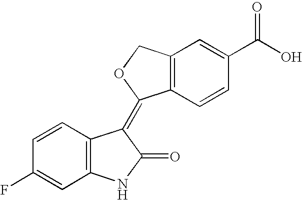 | 1-(6-Fluoro-2-oxo-1,2- dihydro-indol-3-ylidene)-1,3- dihydro-isobenzofuran-5- carboxylic acid | 311.267 |
| 516 | 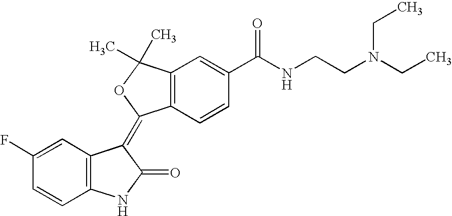 | 1-(5-Fluoro-2-oxo-1,2- dihydro-indol-3-ylidene)-3,3- dimethyl-1,3-dihydro- isobenzofuran-5-carboxylic acid (2-diethylamino- ethyl)-amide | 437.512 |
| 517 | 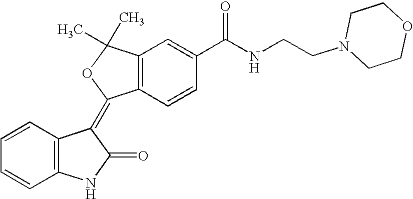 | 3,3-Dimethyl-1-1(2-oxo-1,2- dihydro-indol-3-ylidene)-1,3- dihydro-isobenzofuran-5- carboxylic acid (2-morpholin- 4-yl-ethyl)-amide | 433.505 |
| 518 | 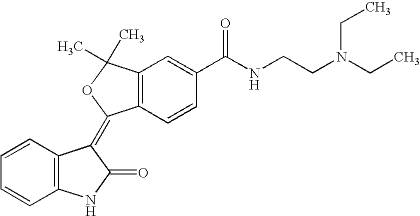 | 3,3-Dimethyl-1-(2-oxo-1,2- dihydro-indol-3-ylidene)-1,3- dihydro-isobenzofuran-5- carboxylic acid (2- diethylamino-ethyl)-amide | 419.522 |
| 519 | 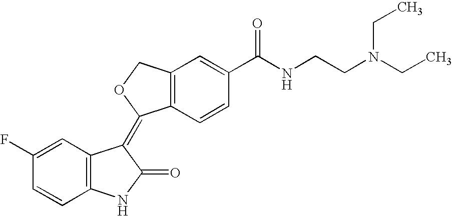 | 1-(5-Fluoro-2-oxo-1,2- dihydro-indol-3-ylidene)-1,3- dihydro-isobenzofuran-5- carboxylic acid (2- diethylamino-ethyl)-amide | 409.459 |
| 520 | 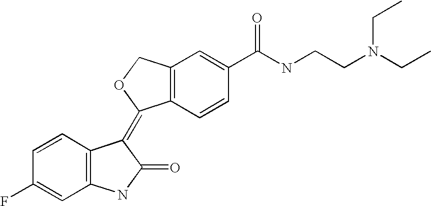 | 1-(6-Fluoro-2-oxo-1,2- dihydro-indol-3-ylidene)-1,3- dihydro-isobenzofuran-5- carboxylic acid (2- diethylamino-ethyl)-amide | 409.459 |
| 521 | 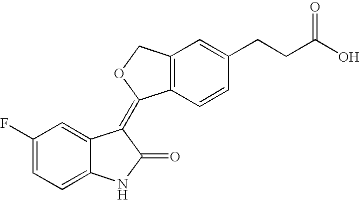 | 3-[1-(5-Fluoro-2-oxo-1,2- dihydro-indol-3-ylidene)-1,3- dihydro-isobenzofuran-5-yl]- propionic acid | 339.321 |
| 522 | 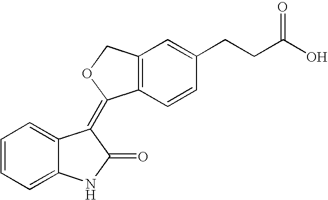 | 3-[1-(2-Oxo-1,2-dihydro-indol- 3-ylidene)-1,3-dihydro- isobenzofuran-5-yl]- propionic acid | 321.331 |
| 523 | 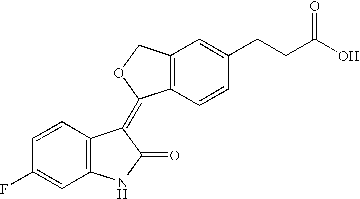 | 3-[1-(6-Fluoro-2-oxo-1,2- dihydro-indol-3-ylidene)-1,3- dihydro-isobenzofuran-5-yl]- propionic acid | 339.321 |
| 524 | 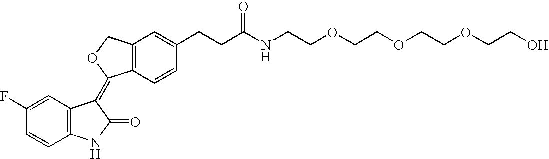 | 3-[1-(5-Fluoro-2-oxo-1,2- dihydro-indol-3-ylidene)-1,3- dihydro-isobenzofuran-5-yl]- N-(2-{2-[2-(2-hydroxy-ethoxy)- ethoxy]-ethoxy}-ethyl)- propionamide | 514.547 |
| 525 | 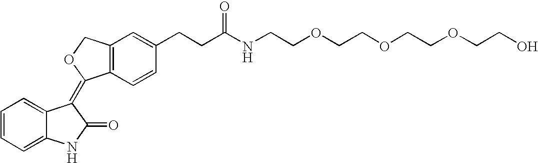 | N-(2-{2-[2-(2-Hydroxy- ethoxy)-ethoxy]-ethoxy}- ethyl)-3-[1-(2-oxo-1,2-dihydro- indol-3-ylidene)-1,3-dihydro- isobenzofuran-5-yl]- propionamide | 496.557 |
| 526 | 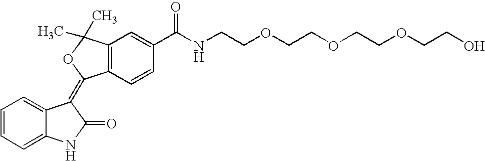 | 3,3-Dimethyl-1-(2-oxo-1,2- dihydro-indol-3-ylidene)-1,3- dihydro-isobenzofuran-5- carboxylic acid (2-{2-[(2- hydroxy-ethoxy)-ethoxy]- ethoxy}-ethyl)-amide | 496.557 |
| 527 | 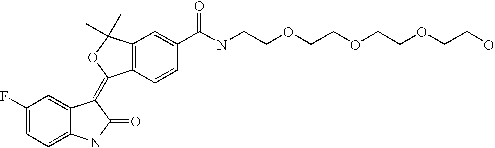 | 1-(5-Fluoro-2-oxo-1,2- dihydro-indol-3-ylidene)-3,3- dimethyl-1,3-dihydro- isobenzofuran-5-carboxylic acid (2-{2-[2-(2-hydroxy- ethoxy)-ethoxy]-ethoxy}- ethyl)-amide | 514.547 |
| 528 | 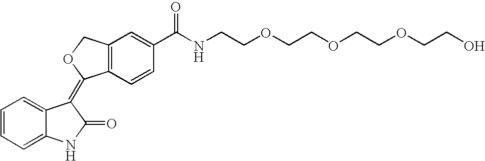 | 1-(2-Oxo-1,2-dihydro-indol-3- ylidene)-1,3-dihydro- isobenzofuran-5-carboxylic acid (2-{2-[2-(2-hydroxy- ethoxy)-ethoxy]-ethoxy}- ethyl)-amide | 468.503 |
| 529 | 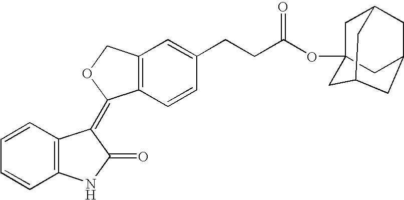 | 3-[1-(2-Oxo-1,2-dihydro-indol- 3-ylidene)-1,3-dihydro- isobenzofuran-5-yl]-propionic acid adamantan-1-yl ester | 455.551 |
| 530 | 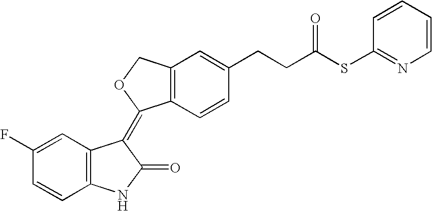 | 3-[1-(5-Fluoro-2-oxo-1,2- dihydro-indol-3-ylidene)-1,3- dihydro-isobenzofuran-5-yl]- thiopropionic acid S-pyridin- 2-yl ester | 432.473 |
| 531 | 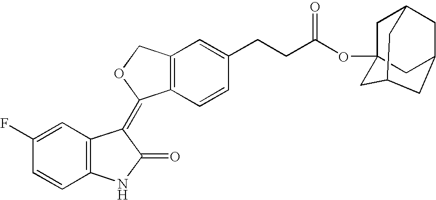 | 3-[1-(5-Fluoro-2-oxo-1,2- dihydro-indol-3-ylidene)-1,3- dihydro-isobenzofuran-5-yl]- propionic acid adamantan-1- yl ester | 473.541 |
| 532 | 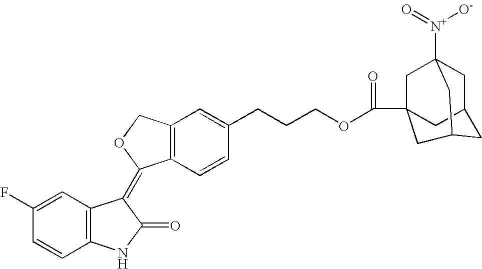 | 3-Nitro-adamantane-1- carboxylic acid 3-[1-(5-fluoro- 2-oxo-1,2-dihydro- indol-3-ylidene)-1,3-dihydro- isobenzofuran-5-yl]- propyl ester | 532.565 |
| 533 | 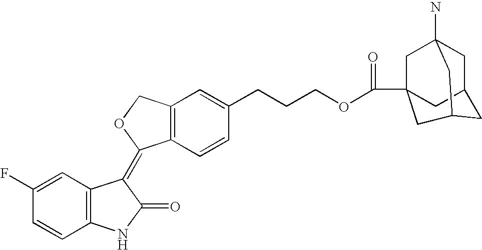 | 3-Amino-adamantane-1- carboxylic acid 3-[1-(5-fluoro- 2-oxo-1,2-dihydro- indol-3-ylidene)-1,3-dihydro- isobenzofuran-5-yl]- propyl ester | 502.583 |
| 534 | 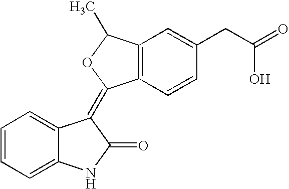 | [3-Methyl-1-(2-oxo-1,2- dihydro-indo[3-ylidene)-1,3- dihydro-isobenzofuran-5-yl]- acetic acid | 321.331 |
| 535 | 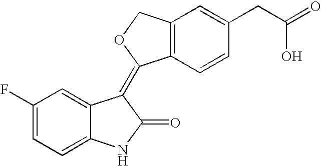 | [1-(5-Fluoro-2-oxo-1,2- dihydro-indol-3-ylidene)-1,3- dihydro-isobenzofuran-5-yl]- acetic acid | 325.294 |
| 536 | 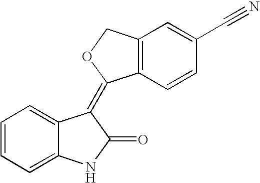 | 1-(2-Oxo-1,2-dihydro-indol-3- ylidene)-1,3-dihydro- isobenzofuran-5-carbonitrile | 274.278 |
| 537 | 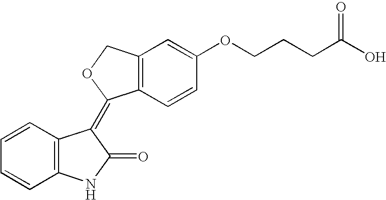 | 4-[1-(2-Oxo-1,2-dihydro-indol- 3-ylidene)-1,3-dihydro- isobenzofuran-5-yloxy]- butyric acid | 351.356 |

TABLE 12

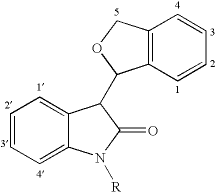

| Example | | | Molecular |
|---|---|---|---|
| Number | Chemical Structure | Chemical Name | Weight |
| 538 | 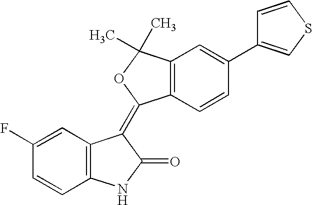 | 3-(3,3-Dimethyl-5-thiophen-3- yl-3H-isobenzofuran-1- ylidene)-5-fluoro-1,3-dihydro- indol-2-one | 377.437 |
| 539 | 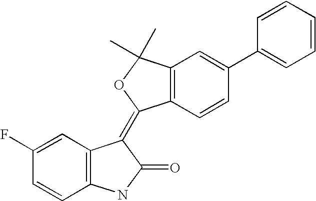 | 3-(3,3-Dimethyl-5-phenyl-3H- isobenzofuran-1-ylidene)-5- fluoro-1,3-dihydro-indol-2-one | 371.409 |

### EXAMPLES

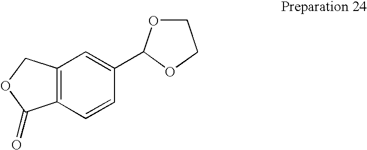

### Preparation of 5-[1,3]dioxolan-2-yl-3H-isobenzofuran-1-one

A mixture of 5-formyl phthalide (1.00 g, 6.17 mmol), ethylene glycol (1 ml, 17.9 mmol) and p-TsOH.H₂O (100 mg) in 25 ml of toluene was refluxed under nitrogen using a Dean-Stark apparatus. The reaction mixture was cooled to room temperature, diluted with 100 ml of EtOAc, washed with saturated NaHCO₃ (2×50 ml) and brine (50 ml), dried over Na₂SO₄, and evaporated. The resulting residue was recrystallized from EtOAc/hexanes to give the title compound as a white solid (1.10 g, 87%).

¹H NMR (500 MHz, DMSO-d₆) δ ppm 3.98-4.11 (m, 4H) 5.43 (s, 2H) 5.90 (s, 1H) 7.64 (d, J=7.81 Hz, 1H) 7.74 (s, 1H) 7.87 (d, J=7.81 Hz, 1H)

### Example 140

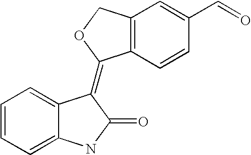

### 1-(2-Oxo-1,2-dihydro-indol-3-ylidene)-1,3-dihydro-isobenzofuran-5-carbaldehyde

To a stirred solution of oxindole (644 mg, 4.84 mmol) in anhydrous THF (10 ml) under nitrogen was added 1.0M LiHMDS/THF solution (9.7 ml, 9.7 mmol). The mixture was stirred at room temperature for 10 minutes, and 5-[1,3]dioxolan-2-yl-3H-isobenzofuran-1-one (500 mg, 2.42 mmol) was added. After stirring at room temperature for 1 hour, the mixture was poured into 15 ml of 2M HCl, heated at 65° C. for 1 hour, and poured into 200 ml of water. The resulting solid was filtered, washed with water, and dried in vacuum to give the title compound as orange/yellow powder (609 mg, 91%).

¹H NMR (500 MHz, DMSO-d₆) δ ppm 5.89 (s, 2H) 6.85 (d, J=7.81 Hz, 1H) 6.99 (t, J=7.57 Hz, 1H) 7.16 (t, J=7.57 Hz, 1H) 7.87 (d, J=7.81 Hz, 1H) 8.10 (d, J=8.30 Hz, 1H) 8.15 (s, 1H) 9.83 (d, J=8.30 Hz, 1H) 10.15 (s, 1H) 10.54 (s, 1H)

### Example 141

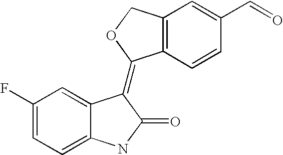

### 1-(5-Fluoro-2-oxo-1,2-dihydro-indol-3-ylidene)-1,3-dihydro-isobenzofuran-5-carbaldehyde

The title compound was prepared in an experimental procedure similar to Example 140.

### Example 142

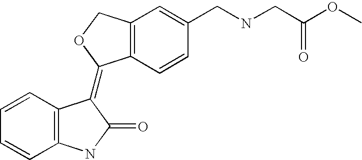

### {[1-(2-Oxo-1,2-dihydro-indol-3-ylidene)-1,3-dihydro-isobenzofuran-5-ylmethyl]-amino}-acetic acid methyl ester

A mixture of 1-(2-oxo-1,2-dihydro-indol-3-ylidene)-1,3-dihydro-isobenzofuran-5-carbaldehyde (100 mg, 0.36 mmol), glycine methyl ester hydrochloride (100 mg, 0.80 mmol), triethylamine (73 mg, 0.72 mmol) and 100 mg of 4A molecular sieves in 5 ml of anhydrous DMF was stirred under nitrogen overnight. Acetic acid (30 mg) and sodium cyanoborohydride (45 mg, 0.72 mmol) were then added. After diluted with 5 ml of anhydrous methanol and stirred at room temperature for 30 minutes, the mixture was filtered through celite, and washed with 3 ml of methanol. The filtrate solution was poured into 150 ml of water with stirring, and basified to approximately pH=8 with saturated NaHCO₃ solution. The resulting precipitates were filtered, washed with water, and dried under vacuum to give the title compound as a yellow solid (96 mg, 76%).

¹H NMR (500 MHz, DMSO-d₆) δ ppm 2.70 (br. s., 1H) 3.37 (s, 2H) 3.64 (s, 3H) 3.87 (s, 2H) 5.79 (s, 2H) 6.83 (d, J=7.32 Hz, 1H) 6.96 (t, J=7.57 Hz, 1H) 7.11 (t, J=7.57 Hz, 1H) 7.50 (d, J=8.30 Hz, 1H) 7.61 (s, 1H) 7.83 (d, J=7.81 Hz, 1H) 9.57 (d, J=7.81 Hz, 1H) 10.40 (s, 1H)

The following Examples 143-256 were prepared using the experimental procedure described in Example 142, but with the appropriate reagent, reaction conditions and reactant substitutions that will be readily realized by those of ordinary skill in this art without the exercise of undue experimentation.

### Example 143

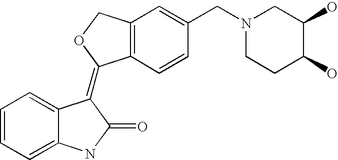

### cis-3-[5-(3,4-Dihydroxy-piperidin-1-ylmethyl)-3H-isobenzofuran-1-ylidene]-1,3-dihydro-indol-2-one

¹H NMR (500 MHz, DMSO-d₆) δ ppm 1.53-1.62 (m, 1H) 1.64-1.72 (m, 1H) 2.29-2.44 (m, 4H) 3.49-3.69 (m, 4H) 4.25 (d, J=2.93 Hz, 1H) 4.35 (d, J=5.86 Hz, 1H) 5.80 (s, 2H) 6.83 (d, J=7.32 Hz, 1H) 6.96 (td, J=7.57, 0.98 Hz, 1H) 7.11 (td, J=7.69, 1.22 Hz, 1H) 7.50 (d, J=8.30 Hz, 1H) 7.59 (s, 1H) 7.83 (d, J=7.81 Hz, 1H) 9.58 (d, J=8.30 Hz, 1H) 10.40 (s, 1H)

### Example 144

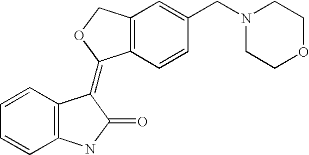

### 3-(5-Morpholin-4-ylmethyl-3H-isobenzofuran-1-ylidene)-1,3-dihydro-indol-2-one

¹H NMR (500 MHz, DMSO-d₆) δ ppm 2.40 (br. s., 4H) 3.58-3.63 (m, 6H) 5.80 (s, 2H) 6.82 (d, J=7.81 Hz, 1H) 6.96 (td, J=7.57, 0.98 Hz, 1H) 7.11 (td, J=7.69, 1.22 Hz, 1H) 7.51 (d, J=7.81 Hz, 1H) 7.60 (s, 1H) 7.83 (d, J=7.81 Hz, 1H) 9.59 (d, J=8.30 Hz, 1H) 10.40 (s, 1H)

### Example 145

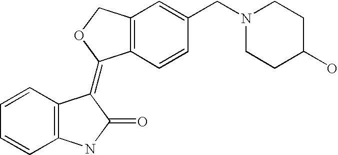

### 3-[5-(4-Hydroxy-piperidin-1-ylmethyl)-3H-isobenzofuran-1-ylidene]-1,3-dihydro-indol-2-one

¹H NMR (500 MHz, DMSO-d₆) δ ppm 1.42 (q, J=9.28 Hz, 2H) 1.72 (d, J=10.25 Hz, 2H) 2.09 (t, J=9.28 Hz, 2H) 2.65-2.78 (m, J=9.76 Hz, 2H) 3.42-3.52 (m, 1H) 3.58 (s, 2H) 4.56 (d, J=3.91 Hz, 1H) 5.80 (s, 2H) 6.83 (d, J=7.32 Hz, 1H) 6.96 (td, J=7.57, 0.98 Hz, 1H) 7.11 (td, J=7.69, 1.22 Hz, 1H) 7.48 (d, J=7.81 Hz, 1H) 7.58 (s, 1H) 7.83 (d, J=7.81 Hz, 1H) 9.58 (d, J=8.30 Hz, 1H) 10.40 (s, 1H)

### Example 146

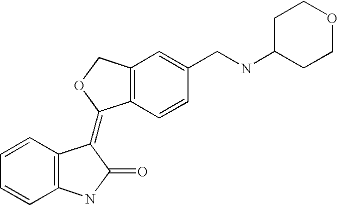

### 3-{5-[(Tetrahydro-pyran-4-ylamino)-methyl]-3H-isobenzofuran-1-ylidene}-1,3-dihydro-indol-2-one

¹H NMR (500 MHz, DMSO-d₆) δ ppm 1.25-1.33 (m, 2H) 1.79 (dd, J=12.69, 1.95 Hz, 2H) 2.26 (br. s., 1H) 2.58-2.64 (m, 1H) 3.26 (td, J=11.47, 1.95 Hz, 2H) 3.82 (t, J=3.42 Hz, 1H) 3.84 (t, J=3.42 Hz, 1H) 3.88 (s, 2H) 5.79 (s, 2H) 6.82 (d, J=7.32 Hz, 1H) 6.96 (td, J=7.57, 0.98 Hz, 1H) 7.10 (td, J=7.69, 1.22 Hz, 1H) 7.53 (d, J=7.81 Hz, 1H) 7.64 (s, 1H) 7.83 (d, J=7.32 Hz, 1H) 9.56 (d, J=8.30 Hz, 1H) 10.39 (s, 1H)

### Example 147

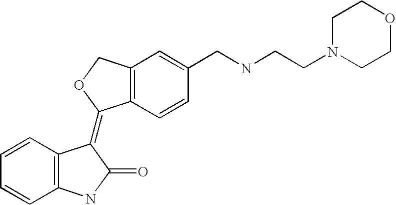

### 3-{5-[(2-Morpholin-4-yl-ethylamino)-methyl]-3H-isobenzofuran-1-ylidene}-1,3-dihydro-indol-2-one

¹H NMR (500 MHz, DMSO-d₆) δ ppm 2.43 (br. s., 4H) 2.58 (t, J=5.61 Hz, 2H) 3.08 (t, J=5.61 Hz, 2H) 3.61 (br. s., 4H) 4.32 (s, 2H) 5.86 (s, 2H) 6.84 (d, J=7.81 Hz, 1H) 6.98 (t, J=7.57 Hz, 1H) 7.14 (t, J=7.57 Hz, 1H) 7.69 (d, J=8.30 Hz, 1H) 7.74 (s, 1H) 7.85 (d, J=7.32 Hz, 1H) 9.69 (d, J=8.30 Hz, 1H) 10.46 (s, 1H)

| Example | | | Molecular | Table |
|---|---|---|---|---|
| Number | Chemical Structure | Chemical Name | Weight | Number |
| 148 | 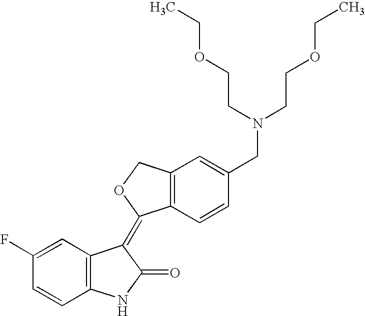 | 3-(5-{[Bis-(2-ethoxy- ethyl)-amino]-methyl}- 3H-isobenzofuran-1- ylidene)-5-fluoro-1,3- dihydro-indol-2-one | 440.512 | 7 |
| 149 | 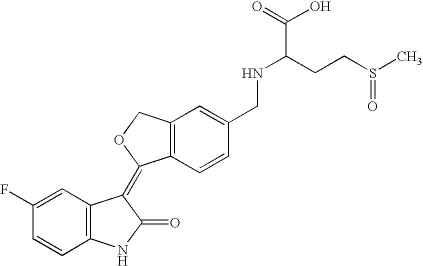 | 2-{[1-(5-Fluoro-2-oxo- 1,2-dihydro-indol-3- ylidene)-1,3-dihydro- isobenzofuran-5- ylmethyl]-amino}-4- methanesulfinyl-butyric acid | 444.481 | 7 |
| 150 | 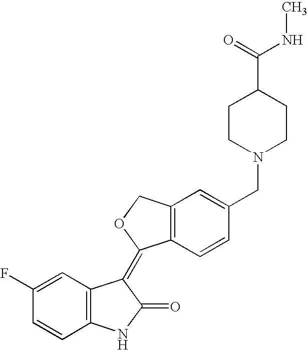 | 1-[1-(5-Fluoro-2-oxo-1,2- dihydro-indol-3-ylidene)- 1,3-dihydro- isobenzofuran-5- ylmethyl]-piperidine-4- carboxylic acid methylamide | 421.47 | 7 |
| 151 | 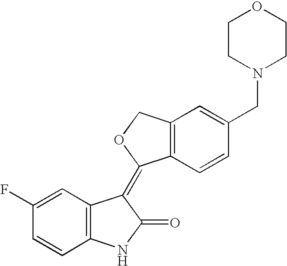 | 5-Fluoro-3-(5-morpholin- 4-ylmethyl-3H- isobenzofuran-1- ylidene)-1,3-dihydro- indol-2-one | 366.39 | 7 |
| 152 | 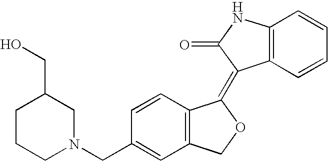 | 3-[5-(3-Hydroxymethyl- piperidin-1-ylmethyl)-3H- isobenzofuran-1- ylidene]-1,3-dihydro- indol-2-one | 376.454 | 7 |
| 153 | 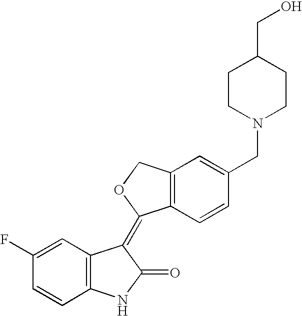 | 5-Fluoro-3-[5-(4- hydroxymethyl-piperidin- 1-ylmethyl)-3H- isobenzofuran-1- ylidene]-1,3-dihydro- indol-2-one | 394.444 | 7 |
| 154 | 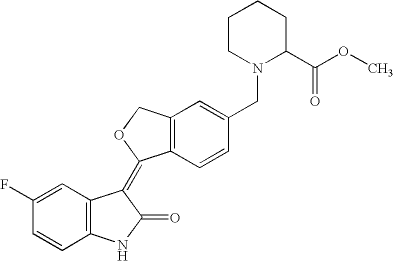 | 1-[1-(5-Fluoro-2-oxo-1,2- dihydro-indol-3-ylidene)- 1,3-dihydro- isobenzofuran-5- ylmethyl]-piperidine-2- carboxylic acid methyl ester | 422.454 | 7 |
| 155 | 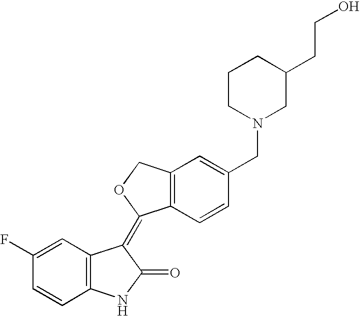 | 5-Fluoro-3-{5-[3-(2- hydroxy-ethyl)-piperidin- 1-ylmethyl]-3H- isobenzofuran-1- ylidene}-1,3-dihydro- indol-2-one | 408.47 | 7 |
| 156 | 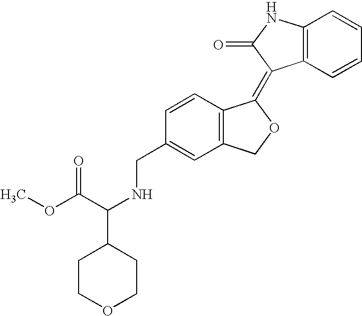 | {[1-(2-Oxo-1,2-dihydro- indol-3-ylidene)-1,3- dihydro-isoberizofuran-5- ylmethyl]-amino}- (tetrahydro-pyran-4-yl)- acetic acid methyl ester | 434.489 | 7 |
| 157 | 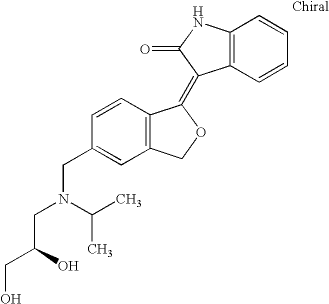 | 3-(5-{[((2S)-2,3- Dihydroxy-propyl)- isopropyl-amino]- methyl}-3H- isobenzofuran-1- ylidene)-1,3-dihydro- indol-2-one | 394.468 | 7 |
| 158 | 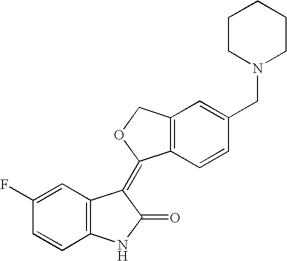 | 5-Fluoro-3-(5-piperidin-1- ylmethyl-3H- isobenzofuran-1- ylidene)-1,3-dihydro- indol-2-one | 364.418 | 7 |
| 159 | 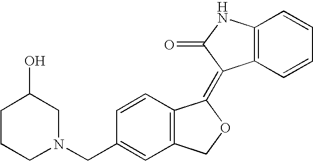 | 3-[5-(3-Hydroxy- piperidin-1-ylmethyl)-3H- isobenzofuran-1- ylidene]-1,3-dihydro- indol-2-one | 362.427 | 7 |
| 160 | 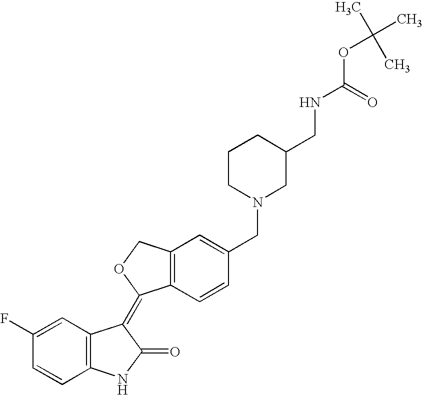 | {1-[1-(5-Fluoro-2-oxo- 1,2-dihydro-indol-3- ylidene)-1,3-dihydro- isobenzofuran-5- ylmethyl]-piperidin-3- ylmethyl}-carbamic acid tert-butyl ester | 493.576 | 7 |
| 161 | 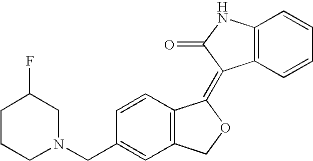 | 3-[5-(3-Fluoro-piperidin- 1-ylmethyl)-3H- isobenzofuran-1- ylidene]-1,3-dihydro- indol-2-one | 364.418 | 7 |
| 162 | 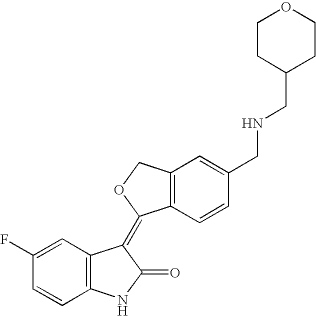 | 5-Fluoro-3-(5- {[(tetrahydro-pyran-4- ylmethyl)-amino]- methyl}-3H- isobenzofuran-1- ylidene)-1,3-dihydro- indol-2-one | 394.444 | 7 |
| 163 | 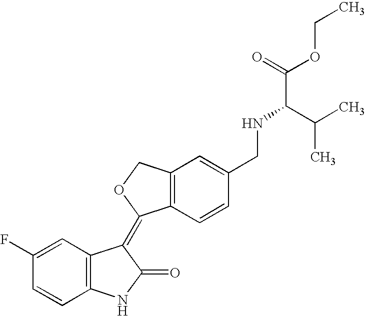 | (S)-2-{[1-(5-Fluoro-2- oxo-1,2-dihydro-indol-3- ylidene)-1,3-dihydro- isobenzofuran-5- ylmethyl]-amino}-3- methyl-butyric acid ethyl ester | 424.47 | 7 |
| 164 | 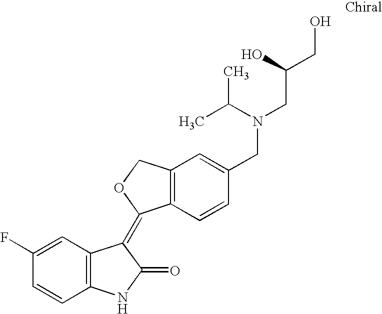 | 3-(5-{[((2R)-2,3- Dihydroxy-propyl)- isopropyl-amino]- methyl}-3H- isobenzofuran-1- ylidene)-5-fluoro-1,3- dihydro-indol-2-one | 412.458 | 7 |
| 165 | 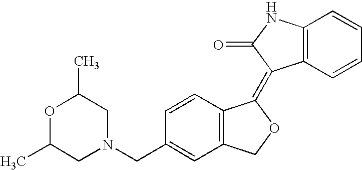 | 3-[5-(2,6-Dimethyl- morpholin-4-ylmethyl)- 3H-isobenzofuran-1- ylidene]-1,3-dihydro- indol-2-one | 376.454 | 7 |
| 166 | 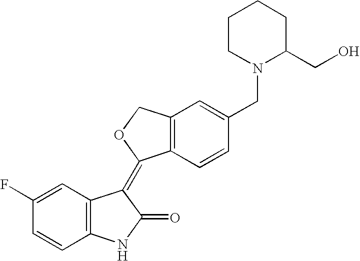 | 5-Fluoro-3-[5-(2- hydroxymethyl-piperidin- 1-ylmethyl)-3H- isobenzofuran-1- ylidene]-1,3-dihydro- indol-2-one | 394.444 | 7 |
| 167 | 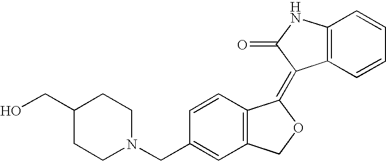 | 3-[5-(4-Hydroxymethyl- piperidin-1-ylmethyl)-3H- isobenzofuran-1- ylidene]-1,3-dihydro- indol-2-one | 373.454 | 7 |
| 168 | 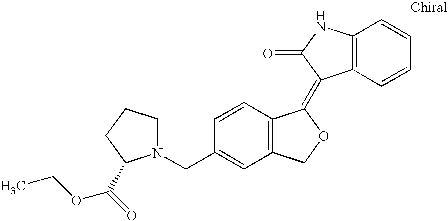 | 1-[1-(2-Oxo-1,2-dihydro- indol-3-ylidene)-1,3- dihydro-isobenzofuran-5- ylmethyl]-pyrrolidine-(S)- 2-carboxylic acid ethyl ester | 404.464 | 7 |
| 169 | 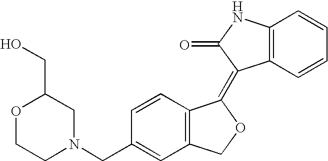 | 3-[5-(2-Hydroxymethyl- morpholin-4-ylmethyl)- 3H-isobenzofuran-1- ylidene]-1,3-dihydro- indol-2-one | 378.426 | 7 |
| 170 | 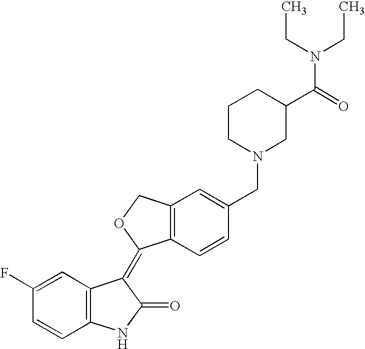 | 1-[1-(5-Fluoro-2-oxo-1,2- dihydro-indol-3-ylidene)- 1,3-dihydro- isobenzofuran-5- ylmethyl]-piperidine-3- carboxylic acid diethylamide | 463.55 | 7 |
| 171 | 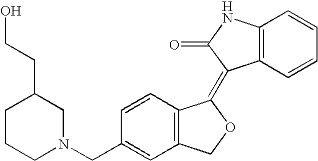 | 3-{5-[3-(2-Hydroxy- ethyl)-piperidin-1- ylmethyl]-3H- isobenzofuran-1- ylidene}-1,3-dihydro- indol-2-one | 390.48 | 7 |
| 172 | 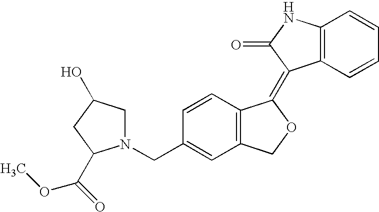 | 4-Hydroxy-1-[1-(2-oxo- 1,2-dihydro-indol-3- ylidene)-1,3-dihydro- isobenzofuran-5- ylmethyl]-pyrrolidine-2- carboxylic acid methyl ester | 406.436 | 7 |
| 173 | 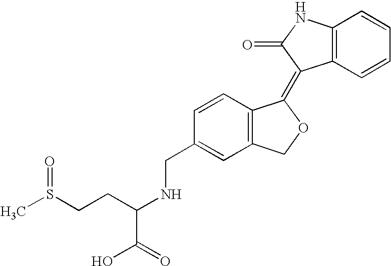 | 4-Methanesulfinyl-2-{[1- (2-oxo-1,2-dihydro-indol- 3-ylidene)-1,3-dihydro- isobenzofuran-5- ylmethyl]-amino}-butyric acid | 426.491 | 7 |
| 174 | 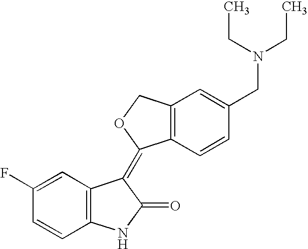 | 3-(5-Diethylaminomethyl- 3H-isobenzofuran-1- ylidene)-5-fluoro-1,3- dihydro-indol-2-one | 352.407 | 7 |
| 175 | 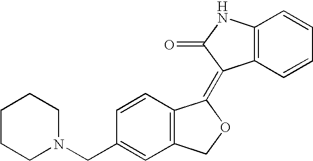 | 3-(5-Piperidin-1-ylmethyl- 3H-isobenzofuran-1- ylidene)-1,3-dihydro- indol-2-one | 346.428 | 7 |
| 176 | 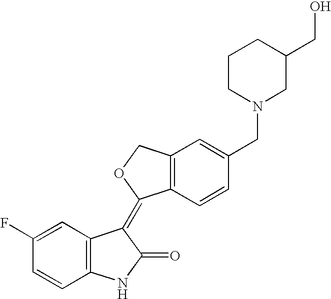 | 5-Fluoro-3-[5-(3- hydroxymethyl-piperidin- 1-ylmethyl)-3H- isobenzofuran-1- ylidene]-1,3-dihydro- indol-2-one | 394.444 | 7 |
| 177 | 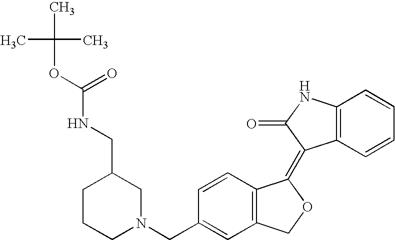 | {1-[1-(2-Oxo-1,2-dihydro- indol-3-ylidene)-1,3- dihydro-isobenzofuran-5- ylmethyl]-piperidin-3- ylmethyl}-carbamic acid tert-butyl ester | 475.586 | 7 |
| 178 | 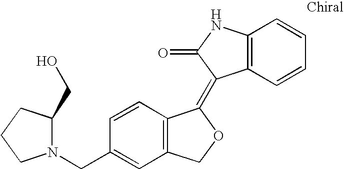 | 3-[5-((S)-2- Hydroxymethyl- pyrrolidin-1-ylmethyl)-3H- isobenzofuran-1- ylidene]-1,3-dihydro- indol-2-one | 362.427 | 7 |
| 179 | 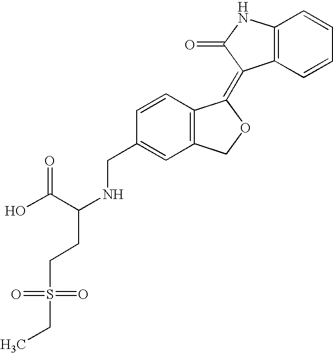 | 4-Ethanesulfonyl-2-{[1- (2-oxo-1,2-dihydro-indol- 3-ylidene)-1,3-dihydro- isobenzofuran-5- ylmethyl]-amino}-butyric acid | 456.517 | 7 |
| 180 | 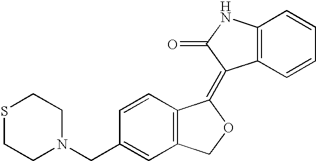 | 3-(5-Thiomorpholin-4- ylmethyl-3H- isobenzofuran-1- ylidene)-1,3-dihydro- indol-2-one | 364.467 | 7 |
| 181 | 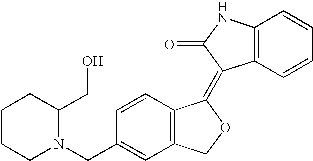 | 3-[5-(2-Hydroxymethyl- piperidin-1-ylmethyl)-3H- isobenzofuran-1- ylidene]-1,3-dihydro- indol-2-one | 376.454 | 7 |
| 182 | 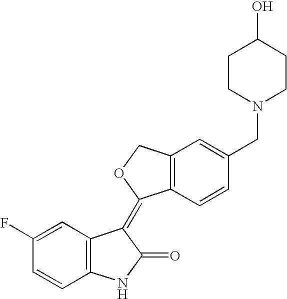 | 5-Fluoro-3-[5-(4-hydroxy- piperidin-1-ylmethyl)-3H- isobenzofuran-1- ylidene]-1,3-dihydro- indol-2-one | 380.417 | 7 |
| 183 | 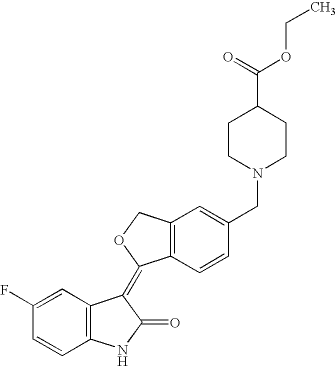 | 1-[1-(5-Fluoro-2-oxo-1,2- dihydro-indol-3-ylidene)- 1,3-dihydro- isobenzofuran-5- ylmethyl]-piperidine-4- carboxylic acid ethyl ester | 436.48 | 7 |
| 184 | 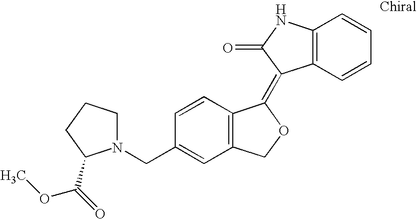 | 1-[1-(2-Oxo-1,2-dihydro- indol-3-ylidene)-1,3- dihydro-isobenzofuran-5- ylmethyl]-pyrrolidine-(S)- 2-carboxylic acid methyl ester | 390.437 | 7 |
| 185 | 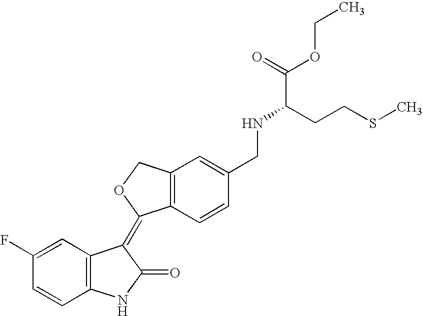 | (S)-2-{[1-(5-Fluoro-2- oxo-1,2-dihydro-indol-3- ylidene)-1,3-dihydro- isobenzofuran-5- ylmethyl]-amino}-4- methylsulfanyl-butyric acid ethyl ester | 456.535 | 7 |
| 186 | 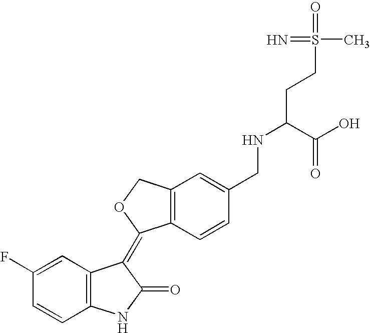 | 2-{[1-(5-Fluoro-2-oxo- 1,2-dihydro-indol-3- ylidene)-1,3-dihydro- isobenzofuran-5- ylmethyl]-amino}-4-(S- methylsulfonimidoyl)- butyric acid | 459.496 | 7 |
| 187 | 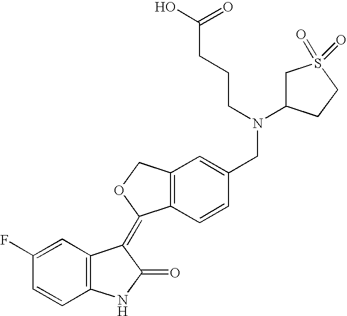 | 4-{(1,1-Dioxo-tetrahydro- 1lambda*6*-thiophen-3- yl)-[1-(5-fluoro-2-oxo-1,2- dihydro-indol-3-ylidene)- 1,3-dihydro- isobenzofuran-5- ylmethyl]-amino}-butyric acid | 500.544 | 7 |
| 188 | 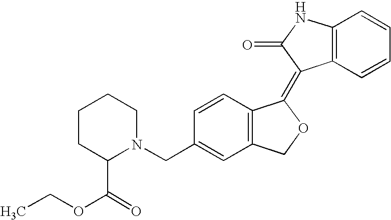 | 1-[1-(2-Oxo-1,2-dihydro- indol-3-ylidene)-1,3- dihydro-isobenzofuran-5- ylmethyl]-piperidine-2- carboxylic acid ethyl ester | 418.49 | 7 |
| 189 | 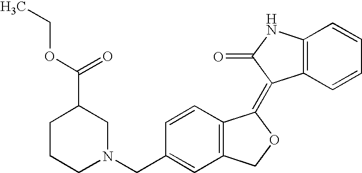 | 1-[1-(2-Oxo-1,2-dihydro- indol-3-ylidene)-1,3- dihydro-isobenzofuran-5- ylmethyl]-piperidine-3- carboxylic acid ethyl ester | 418.49 | 7 |
| 190 | 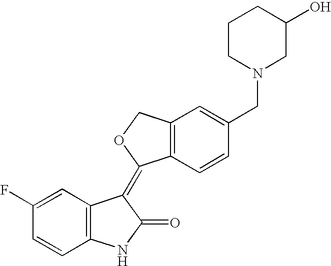 | 5-Fluoro-3-[5-(3-hydroxy- piperidin-1-ylmethyl)-3H- isobenzofuran-1- ylidene]-1,3-dihydro- indol-2-one | 380.417 | 7 |
| 191 | 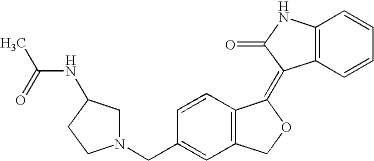 | N-{1-[1-(2-Oxo-1,2- dihydro-indol-3-ylidene)- 1,3-dihydro- isobenzofuran-5- ylmethyl]-pyrrolidin-3-yl}- acetamide | 389.453 | 7 |
| 192 | 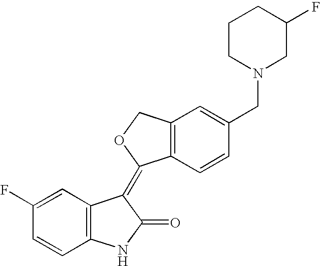 | 5-Fluoro-3-[5-(3-fluoro- piperidin-1-ylmethyl)-3H- isobenzofuran-1- ylidene]-1,3-dihydro- indol-2-one | 382.408 | 7 |
| 193 | 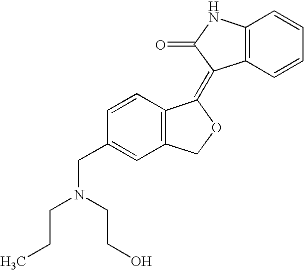 | 3-(5-{[(2-Hydroxy-ethyl)- propyl-amino]-methyl}- 3H-isobenzofuran-1- ylidene)-1,3-dihydro- indol-2-one | 364.443 | 7 |
| 194 | 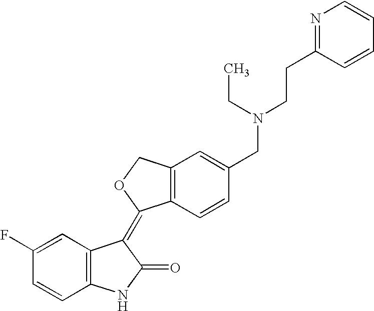 | 3-(5-{[Ethyl-(2-pyridin-2- yl-ethyl)-amino]-methyl}- 3H-isobenzofuran-1- ylidene)-5-fluoro-1,3- dihydro-indol-2-one | 429.493 | 7 |
| 195 | 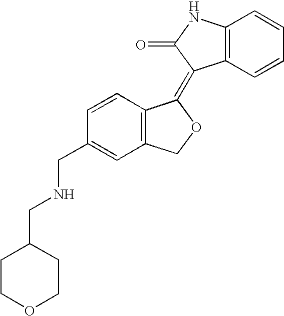 | 3-(5-{[(Tetrahydro-pyran- 4-ylmethyl)-amino]- methyl}-3H- isobenzofuran-1- ylidene)-1,3-dihydro- indol-2-one | 376.454 | 7 |
| 196 | 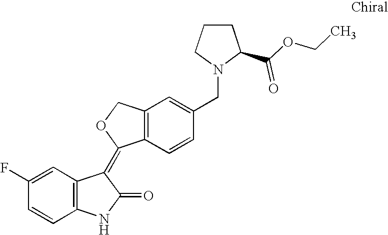 | 1-[1-(5-Fluoro-2-oxo-1,2- dihydro-indol-3-ylidene)- 1,3-dihydro- isobenzofuran-5- ylmethyl]-pyrrolidine-(S)- 2-carboxylic acid ethyl ester | 422.454 | 7 |
| 197 | 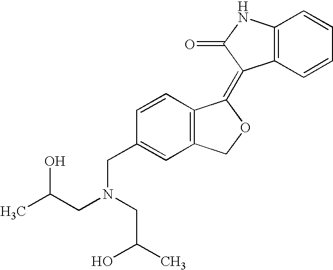 | 3-(5-{[Bis-(2-hydroxy- propyl)-amino]-methyl}- 3H-isobenzofuran-1- ylidene)-1,3-dihydro- indol-2-one | 394.468 | 7 |
| 198 | 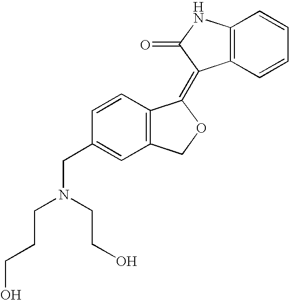 | 3-(5-{[(2-Hydroxy-ethyl)- (3-hydroxy-propyl)- amino]-methyl}-3H- isobenzofuran-1- ylidene)-1,3-dihydro- indol-2-one | 380.442 | 7 |
| 199 | 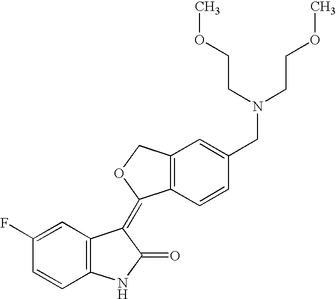 | 3-(5-{[Bis-(2-methoxy- ethyl)-amino]-methyl}- 3H-isobenzofuran-1- ylidene)-5-fluoro-1,3- dihydro-indol-2-one | 412.458 | 7 |
| 200 | 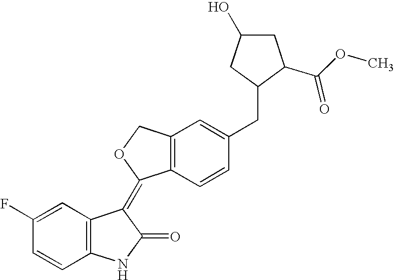 | 1-[1-(5-Fluoro-2-oxo-1,2- dihydro-indol-3-ylidene)- 1,3-dihydro- isobenzofuran-5- ylmethyl]-4-hydroxy- pyrrolidine-2-carboxylic acid methyl ester | 424.426 | 7 |
| 201 | 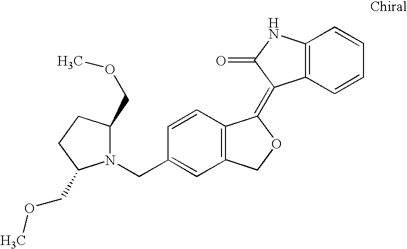 | 3-[5-((S,S)-2,5-Bis- methoxymethyl- pyrrolidin-1-ylmethyl)-3H- isobenzofuran-1- ylidene]-1,3-dihydro- indol-2-one | 420.506 | 7 |
| 202 | 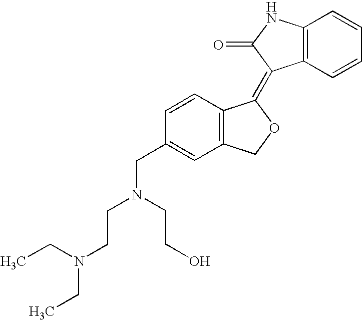 | 3-(5-{[(2-Diethylamino- ethyl)-(2-hydroxy-ethyl)- amino]-methyl}-3H- isobenzofuran-1- ylidene)-1,3-dihydro- indol-2-one | 421.538 | 7 |
| 203 | 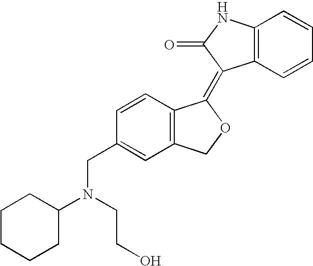 | 3-(5-{[Cyclohexyl-(2- hydroxy-ethyl)-amino]- methyl}-3H- isobenzofuran-1- ylidene)-1,3-dihydro- indol-2-one | 404.507 | 7 |
| 204 | 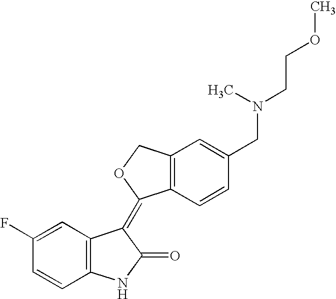 | 5-Fluoro-3-(5-{[(2- methoxy-ethyl)-methyl- amino]-methyl}-3H- isobenzofuran-1- ylidene)-1,3-dihydro- indol-2-one | 368.406 | 7 |
| 205 | 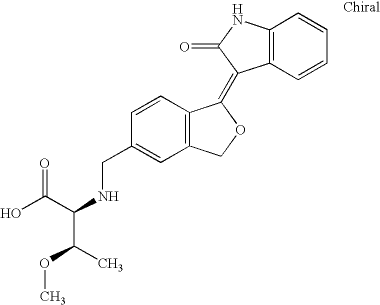 | (R)-3-Methoxy-(S)-2-{[1- (2-oxo-1,2-dihydro-indol- 3-ylidene)-1,3-dihydro- isobenzofuran-5- ylmethyl]-aminol-butyric acid | 394.425 | 7 |
| 206 | 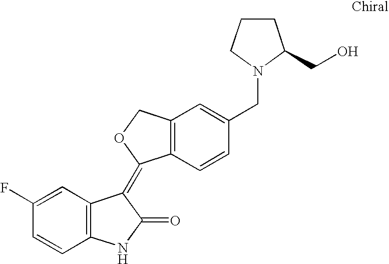 | 5-Fluoro-3-{5-[(S)-2- hydroxymethyl-pyrrolidin- 1-ylmethyl}-3H- isobenzofuran-1- ylidene]-1,3-dihydro- indol-2-one | 380.417 | 7 |
| 207 | 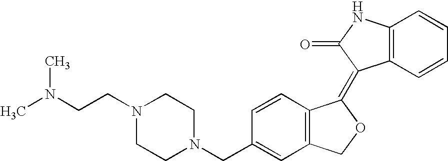 | 3-{5-[4-(2- Dimethylamino-ethyl)- piperazin-1-ylmethyl]-3H- isobenzofuran-1- ylidene}-1,3-dihydro- indol-2-one | 418.538 | 7 |
| 208 | 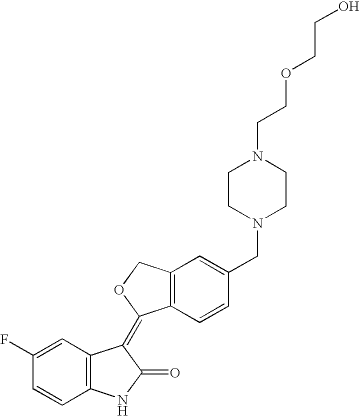 | 5-Fluoro-3-(5-{4-[2-(2- hydroxy-ethoxy)-ethyl]- piperazin-1-ylmethyl}-3H- isobenzofuran-1- ylidene)-1,3-dihydro- indol-2-one | 453.511 | 7 |
| 209 | 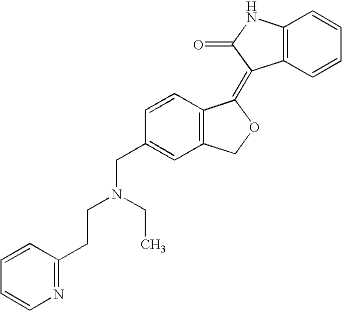 | 3-(5-{[Ethyl-(2-pyridin-2- yl-ethyl)-amino]-methyl}- 3H-isobenzofuran-1- ylidene)-1,3-dihydro- indol-2-one | 411.503 | 7 |
| 210 | 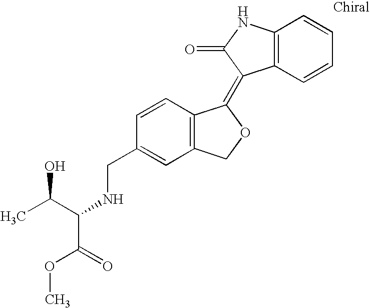 | (R)-3-Hydroxy-(S)-2-{[1- (2-oxo-1,2-dihydro-indol- 3-ylidene)-1,3-dihydro- isobenzofuran-5- ylmethyl]-amino}-butyric acid methyl ester | 394.425 | 7 |
| 211 | 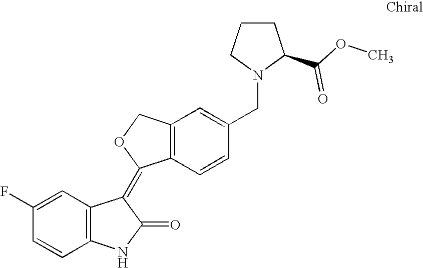 | 1-[1-(5-Fluoro-2-oxo-1,2- dihydro-indol-3-ylidene)- 1,3-dihydro- isobenzofuran-5- ylmethyl]-pyrrolidine-(S)- 2-carboxylic acid methyl ester | 408.427 | 7 |
| 212 | 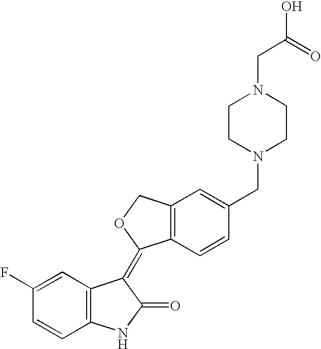 | {4-[1-(5-Fluoro-2-oxo- 1,2-dihydro-indol-3- ylidene)-1,3-dihydro- isobenzofuran-5- ylmethyl]-piperazin-1-yl}- acetic acid | 423.442 | 7 |
| 213 | 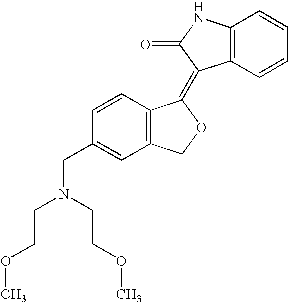 | 3-(5-{[Bis-(2-methoxy- ethyl)-amino]-methyl}- 3H-isobenzofuran-1- ylidene)-1,3-dihydro- indol-2-one | 394.468 | 7 |
| 214 | 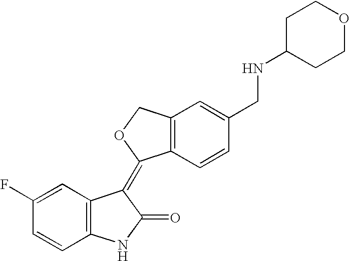 | 5-Fluoro-3-{5- [(tetrahydro-pyran-4- ylamino)-methyl]-3H- isobenzofuran-1- ylidene}-1,3-dihydro- indol-2-one | 380.417 | 7 |
| 215 | 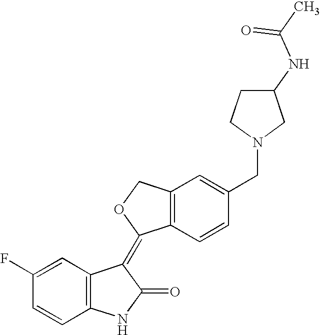 | N-{1-[1-(5-Fluoro-2-oxo- 1,2-dihydro-indol-3- ylidene)-1,3-dihydro- isobenzofuran-5- ylmethyl]-pyrrolidin-3-yl}- acetamide | 407.443 | 7 |
| 216 | 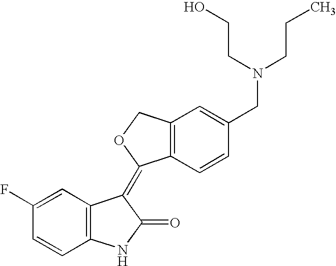 | 5-Fluoro-3-(5-{[(2- hydroxy-ethyl)-propyl- amino]-methyl}-3H- isobenzofuran-1- ylidene)-1,3-dihydro- indol-2-one | 382.433 | 7 |
| 217 | 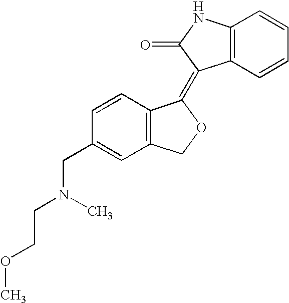 | 3-(5-{[(2-Methoxy-ethyl)- methyl-amino]-methyl}- 3H-isobenzofuran-1- ylidene)-1,3-dihydro- indol-2-one | 350.416 | 7 |
| 218 | 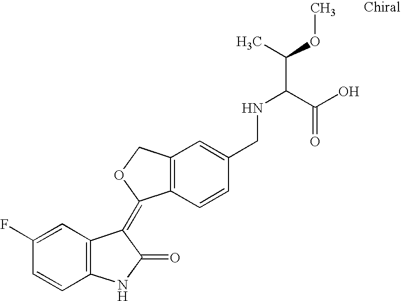 | (S)-2-{[1-(5-Fluoro-2- oxo-1,2-dihydro-indol-3- ylidene)-1,3-dihydro- isobenzofuran-5- ylmethyl]-amino}-(R)-3- methoxy-butyric acid | 412.415 | 7 |
| 219 | 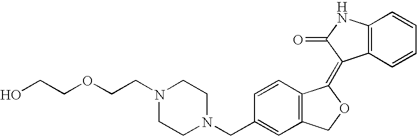 | 3-(5-{4-[2-(2-Hydroxy- ethoxy)-ethyl]-piperazin- 1-ylmethyl}-3H- isobenzofuran-1- ylidene)-1,3-dihydro- indol-2-one | 435.521 | 7 |
| 220 | 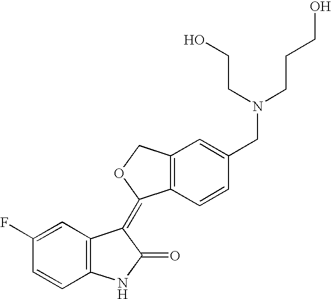 | 5-Fluoro-3-(5-{[(2- hydroxy-ethyl)-(3- hydroxy-propyl)-amino]- methyl}-3H- isobenzofuran-1- ylidene)-1,3-dihydro- indol-2-one | 398.432 | 7 |
| 221 | 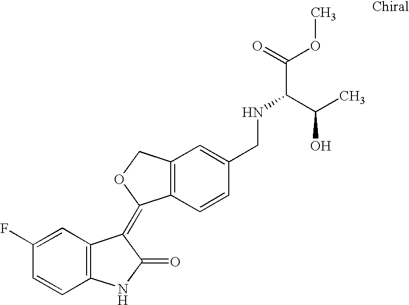 | 6(S)-2-{[1-(5-Fluoro-2- oxo-1,2-dihydro-indol-3- ylidene)-1,3-dihydro- isobenzofuran-5- ylmethyl]-amino}-(R)-3- hydroxy-butyric acid methyl ester | 412.415 | 7 |
| 222 | 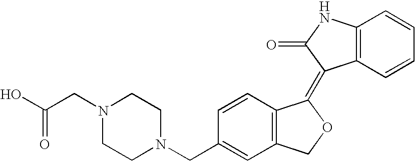 | }4-[1-(2-Oxo-1,2-dihydro- indol-3-ylidene)-1,3- dihydro-isobenzofuran-5- ylmethyl]-piperazin-1-yl}- acetic acid | 405.452 | 7 |
| 223 | 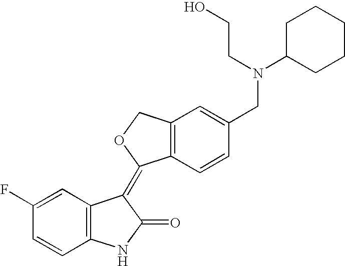 | 3-(5-{[Cyclohexyl-(2- hydroxy-ethyl)-amino]- methyl}-3H- isobenzofuran-1- ylidene)-5-fluoro-1,3- dihydro-indol-2-one | 422.497 | 7 |
| 224 | 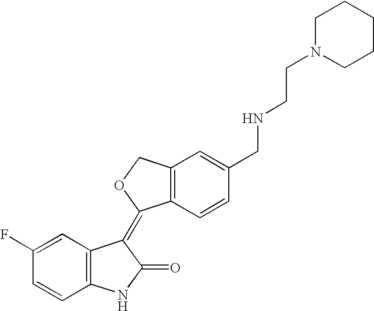 | 5-Fluoro-3-{5-[(2- piperidin-1-yl- ethylamino)-methyl]-3H- isobenzofuran-1- ylidene}-1,3-dihydro- indol-2-one | 407.486 | 7 |
| 225 | 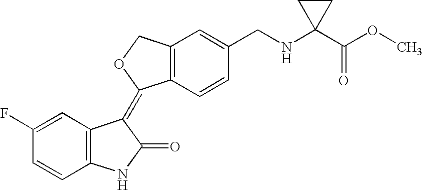 | 1-([1-(5-Fluoro-2-oxo- 1,2-dihydro-indol-3- ylidene)-1,3-dihydro- isobenzofuran-5- ylmethyl]-amino}- cyclopropane- carboxylic acid methyl ester | 394.4 | 7 |
| 226 | 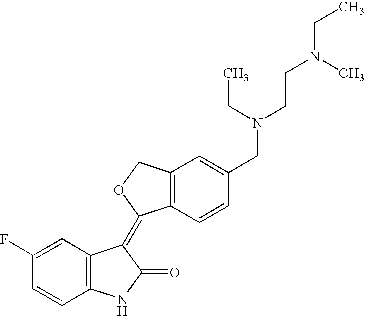 | 3-[5-({Ethyl-[2-(ethyl- methyl-amino)-ethyl]- amino}-methyl)-3H- isobenzofuran-1- ylidene]-5-fluoro-1,3- dihydro-indol-2-one | 409.502 | 7 |
| 227 | 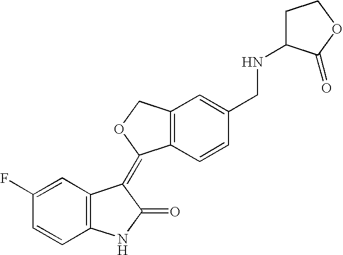 | 5-Fluoro-3-{5-[(2-oxo- tetrahydro-furan-3- ylamino)-methyl]-3H- isobenzofuran-1- ylidene}-1,3-dihydro- indol-2-one | 380.373 | 7 |
| 228 | 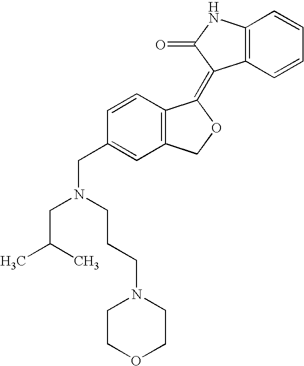 | 3-(5-{[Isobutyl-(3- morpholin-4-yl-propyl)- amino]-methyl}-3H- isobenzofuran-1- ylidene)-1,3-dihydro- indol-2-one | 461.603 | 7 |
| 229 | 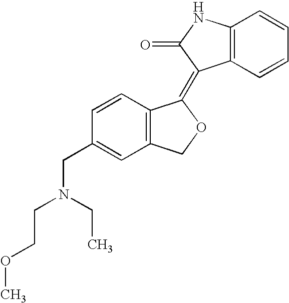 | 3-(5-{[Ethyl-(2-methoxy- ethyl)-amino]-methyl}- 3H-isobenzofuran-1- ylidene)-1,3-dihydro- indol-2-one | 364.443 | 7 |
| 230 | 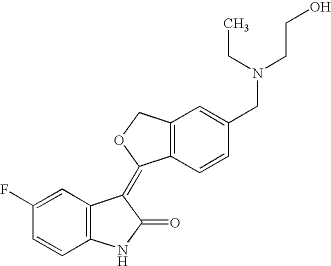 | 3-(5-{[Ethyl-(2-hydroxy- ethyl)-amino]-methyl}- 3H-isobenzofuran-1- ylidene)-5-fluoro-1,3- dihydro-indol-2-one | 368.406 | 7 |
| 231 | 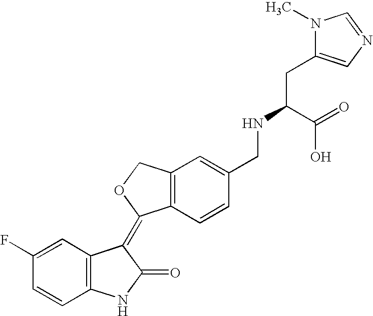 | (S)-2-{[1-(5-Fluoro-2- oxo-1,2-dihydro-indol-3- ylidene)-1,3-dihydro- isobenzofuran-5- ylmethyl]-amino}-3-(3- methyl-3H-imidazol-4-yl)- propionic acid | 448.452 | 7 |
| 232 | 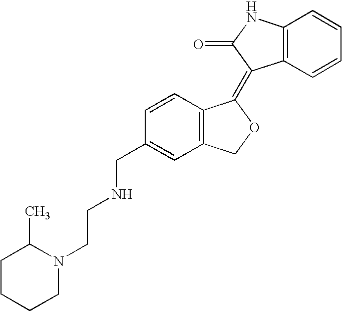 | 3-(5-{[2-(2-Methyl- piperidin-1-yl)- ethylamino]-methyl}-3H- isobenzofuran-1- ylidene)-1,3-dihydro- indol-2-one | 403.523 | 7 |
| 233 | 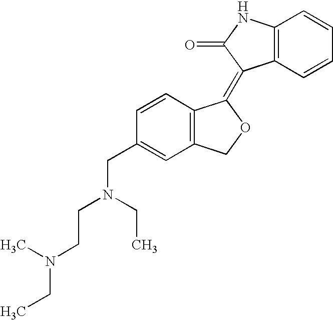 | 3-[5-({Ethyl-[2-(ethyl- methyl-amino)-ethyl]- amino}-methyl)-3H- isobenzofuran-1- ylidene]-1,3-dihydro- indol-2-one | 391.512 | 7 |
| 234 | 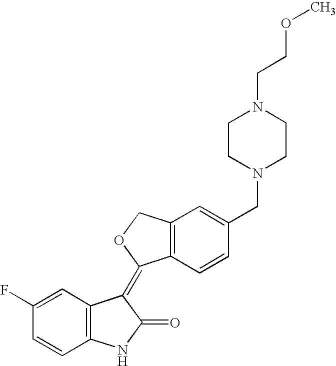 | 5-Fluoro-3-{5-[4-(2- methoxy-ethyl)- piperazin-1-ylmethyl]-3H- isobenzofuran-1- ylidene}-1,3-dihydro- indol-2-one | 423.485 | 7 |
| 235 | 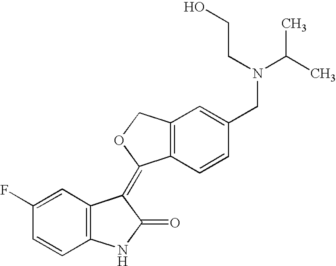 | 5-Fluoro-3-(5-{[(2- hydroxy-ethyl)-isopropyl- amino]-methyl}-3H- isobenzofuran-1- ylidene)-1,3-dihydro- indol-2-one | 382.433 | 7 |
| 236 | 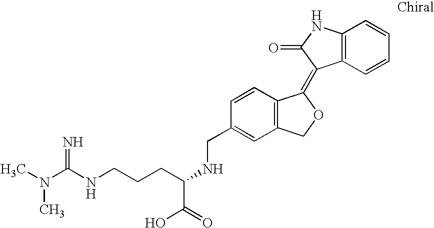 | 5-(N′,N′-Dimethyl- guanidino)-(S)-2-{[1-(2- oxo-1,2-dihydro-indol-3- ylidene)-1,3-dihydro- isobenzofuran-5- ylmethyl]-amino}- pentanoic acid | 463.535 | 7 |
| 237 | 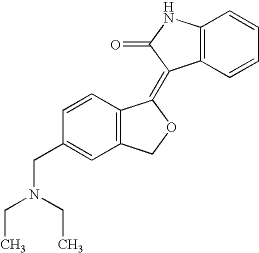 | 3-(5-Diethylaminomethyl- 3H-isobenzofuran-1- ylidene)-1,3-dihydro- indol-2-one | 334.417 | 7 |
| 238 | 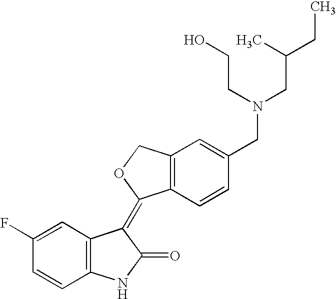 | 5-Fluoro-3-(5-{[(2- hydroxy-ethyl)-(2-methyl- butyl)-amino]-methyl}- 3H-isobenzofuran-1- ylidene)-1,3-dihydro- indol-2-one | 410.486 | 7 |
| 239 | 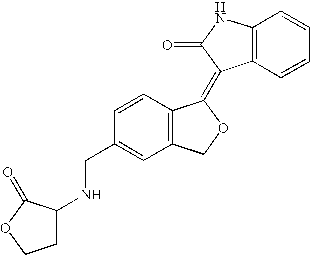 | 3-{5-[(2-Oxo-tetrahydro- furan-3-ylamino)-methyl]- 3H-isobenzofuran-1- ylidene}-1,3-dihydro- indol-2-one | 362.383 | 7 |
| 240 | 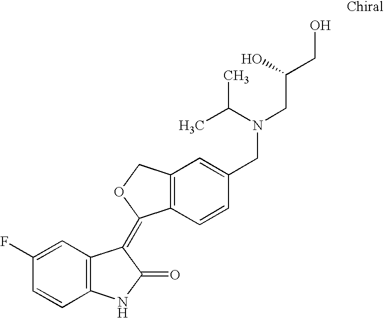 | 3-(5-}[((2S)-2,3- Dihydroxy-propyl)- isopropyl-amino]- methyl}-3H- isobenzofuran-1- ylidene)-5-fluoro-1,3- dihydro-indol-2-one | 412.458 | 7 |
| 241 | 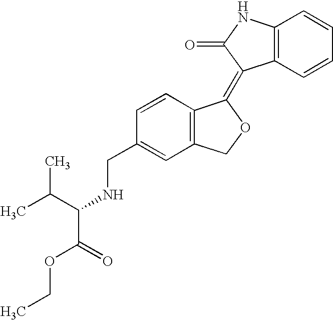 | 3-Methyl-(S)-2-{[1-(2- oxo-1,2-dihydro-indol-3- ylidene)-1,3-dihydro- isobenzofuran-5- ylmethyl]-amino}-butyric acid ethyl ester | 406.479 | 7 |
| 242 | 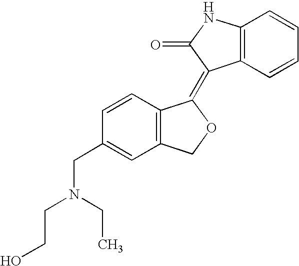 | 3-(5-{[Ethyl-(2-hydroxy- ethyl)-amino]-methyl}- 3H-isobenzofuran-1- ylidene)-1,3-dihydro- indol-2-one | 350.416 | 7 |
| 243 | 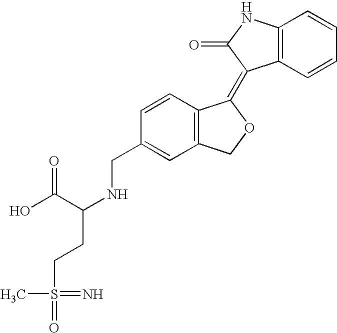 | 4-(S-Methylsulfonimidoyl)-2- {[1-(2-oxo-1,2-dihydro- indol-3-ylidene)-1,3- dihydro-isobenzofuran-5- ylmethyl]-amino}-butyric acid | 441.506 | 7 |
| 244 | 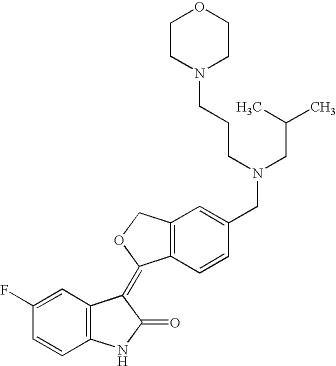 | 5-Fluoro-3-(5-{[isobutyl- (3-morpholin-4-yl- propyl)-amino]-methyl}- 3H-isobenzofuran-1- ylidene)-1,3-dihydro- indol-2-one | 479.593 | 7 |
| 245 | 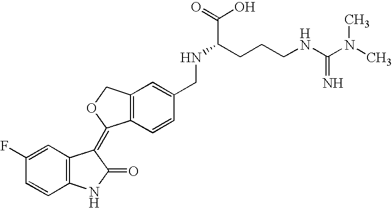 | 5-(N′,N′-Dimethyl- guanidino)-(S)-2-{[1-(5- fluoro-2-oxo-1,2-dihydro- indol-3-ylidene)-1,3- dihydro-isobenzofuran-5- ylethyl]-amino}- pentanoic acid | 481.525 | 7 |
| 246 | 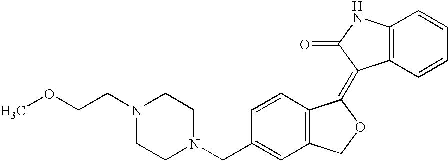 | 3-{5-[4-(2-Methoxy- ethyl)-piperazin-1- ylmethyl]-3H- ylidene}-1,3-dihydro- indol-2-one | 405.495 | 7 |
| 247 | 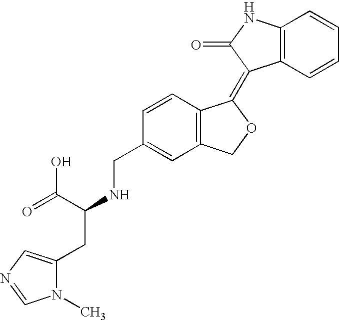 | 3-(3-Methyl-3H-imidazol- 4-yl)-(S)-2-{[1-(2-oxo- 1,2-dihydro-indol-3- ylidene)-1,3-dihydro- isobenzofuran-5- ylmethyl]-amino}- propionic acid | 430.462 | 7 |
| 248 | 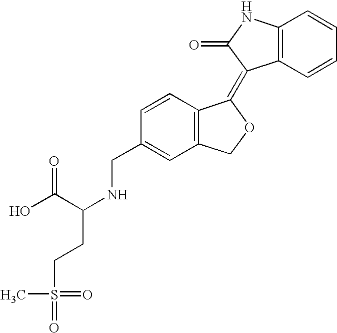 | 4-Methanesulfonyl-2-{[1- (2-oxo-1,2-dihydro-indol- 3-ylidene)-1,3-dihydro- isobenzofuran-5- ylmethyl]-aminol-butyric acid | 442.49 | 7 |
| 249 | 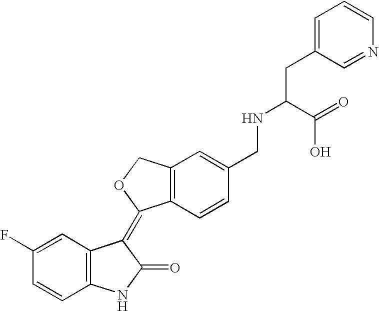 | 2-{[1-(5-Fluoro-2-oxo- 1,2-dihydro-indol-3- ylidene)-1,3-dihydro- isobenzofuran-5- ylmethyl]-amino}-3- pyridin-3-yl-propionic acid | 445.448 | 7 |
| 250 | 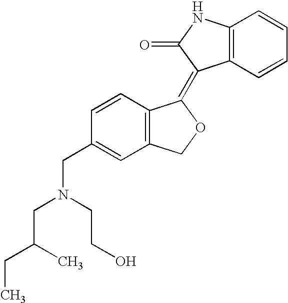 | 3-(5-{[(2-Hydroxy-ethyl)- (2-methyl-butyl)-amino]- methyl}-3H- isobenzofuran-1- ylidene)-1,3-dihydro- indol-2-one | 392.496 | 7 |
| 251 | 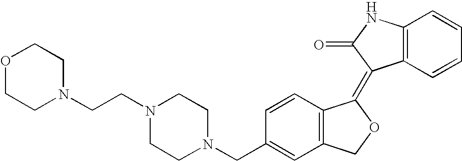 | 3-{5-[4-(2-Morpholin-4-yl- ethyl)-piperazin-1- ylmethyl]-3H- isobenzofuran-1- ylidene}-1,3-dihydro- indol-2-one | 460.575 | 7 |
| 252 | 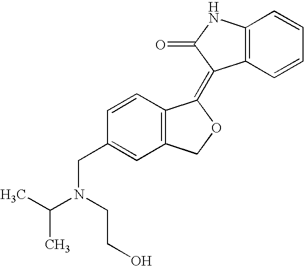 | 3-(5-{[(2-Hydroxy-ethyl)- isopropyl-amino]- methyl}-3H- isobenzofuran-1- ylidene)-1,3-dihydro- indol-2-one | 364.443 | 7 |
| 253 | 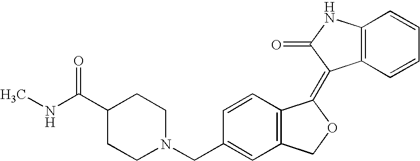 | 1-[1-(2-Oxo-1,2-dihydro- indol-3-ylidene)-1,3- dihydro-isobenzofuran-5- ylmethyl]-piperidine-4- carboxylic acid methylamide | 403.479 | 7 |
| 254 | 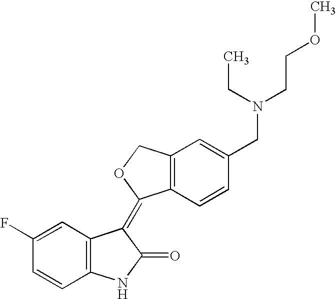 | 3-(5-{[Ethyl-(2-methoxy- ethyl)-amino]-methyl}- 3H-isobenzofuranl-1- ylidene)-5-fluoro-1,3- dihydro-indol-2-one | 382.433 | 7 |
| 255 | 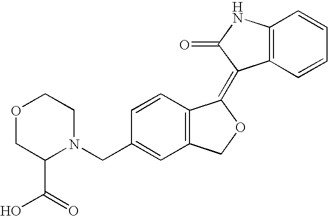 | 4-[1-(2-Oxo-1,2-dihydro- indol-3-ylidene)-1,3- dihydro-isobenzofuran-5- ylmethyl]-morpholine-3- carboxylic acid | 392.409 | 7 |
| 256 | 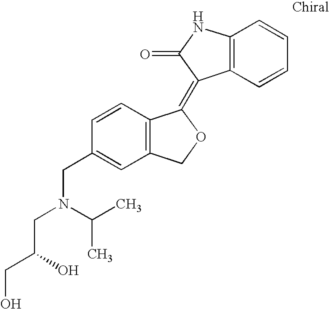 | 3-(5-{[((2R)-2,3- Dihydroxy-propyl)- isopropyl-amino]- methyl}-3H- isobenzofuran-1- ylidene)-1,3-dihydro- indol-2-one | 394.468 | 7 |

### Example 257

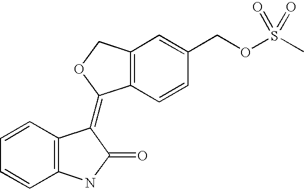

### Methanesulfonic acid 1-(2-oxo-1,2-dihydro-indol-3-ylidene)-1,3-dihydro-isobenzofuran-5-ylmethyl ester

To a stirred suspension of 1-(2-oxo-1,2-dihydro-indol-3-ylidene)-1,3-dihydro-isobenzofuran-5-carbaldehyde (50 mg, 0.18 mmol) in MeOH (10 ml), was added potassium borohydride (20 mg, 0.37 mmol). The mixture was stirred at room temperature for 20 minutes, and poured into 100 ml of water. The precipitates were filtered, washed with water and dried in vacuum to give the benzyl alcohol as a yellow solid (42 mg), which was dissolved in anhydrous THF (5 ml). Triethylamine (60 mg, 0.59 mmol) and methanesulfonyl chloride (0.52 mmol) were added. The mixture was stirred at room temperature for 3 hours and poured into water (75 ml) containing 0.5 ml of AcOH. The precipitates were filtered, washed with water and dried in vacuum to give the title compound as a yellow solid (52 mg, 81% in two steps).

¹H NMR (500 MHz, DMSO-d₆) ppm 3.30 (s, 3H) 5.42 (s, 2H) 5.84 (s, 2H) 6.84 (d, J=7.32 Hz, 1H) 6.97 (td, J=7.57, 0.98 Hz, 1H) 7.13 (td, J=7.69, 1.22 Hz, 1H) 7.63 (d, J=8.30 Hz, 1H) 7.73 (s, 1H) 7.84 (d, J=7.32 Hz, 1H) 9.68 (d, J=7.81 Hz, 1H) 10.45 (s, 1H)

### Example 258

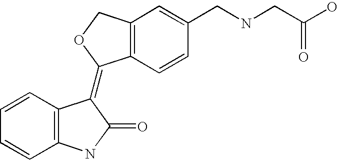

### {[1-(2-Oxo-1,2-dihydro-indol-3-ylidene)-1,3-dihydro-isobenzofuran-5-ylmethyl]-amino}-acetic acid

To a stirred suspension of {[1-(2-oxo-1,2-dihydro-indol-3-ylidene)-1,3-dihydro-isobenzofuran-5-ylmethyl]-amino}-acetic acid methyl ester (55 mg, 0.16 mmol) in MeOH (4 ml) was added 5M NaOH solution (1 ml). The mixture was stirred at room temperature for 45 minutes, and poured into 75 ml of water. Acidification of the mixture with 2M HCl to pH 6 gave the precipitates. The resulting precipitates were filtered, washed with water and dried in vacuum to give the title compound as a brown solid (54 mg, 100%). MS (ES−): 335.1107 (M−H)

### Example 259

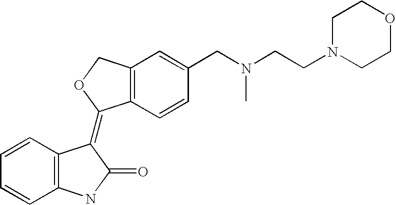

### 3-(5-{[Methyl-(2-morpholin-4-yl-ethyl)-amino]-methyl}-3H-isobenzofuran-1-ylidene)-1,3-dihydro-indol-2-one

A mixture of 3-{5-[(2-morpholin-4-yl-ethylamino)-methyl]-3H-isobenzofuran-1-ylidene}-1,3-dihydro-indol-2-one AcOH salt (100 mg, 0.22 mmol), 37% formaldehyde aqueous solution (0.5 ml), sodium cyanoborohydride (30 mg, 0.48 mmol) and AcOH (0.25 ml) in MeOH (5 ml) was stirred at room temperature for 20 minutes, and then poured into 75 ml of water. The basification of the mixture with saturated NaHCO₃ solution to pH 9 gave the precipitates. The resulting precipitates were filtered, washed with water and dried under vacuum to give the title compound as a yellow solid (82 mg, 91%).

¹H NMR (500 MHz, DMSO-d6) δ ppm 2.19 (s, 3H) 2.37 (br. s., 4H) 2.43-2.48 (m, 2H) 2.49-2.53 (m, 2H) 3.55 (t, J=4.39 Hz, 4H) 3.63 (s, 2H) 5.79 (s, 2H) 6.83 (d, J=7.81 Hz, 1H) 6.96 (td, J=7.57, 0.98 Hz, 1H) 7.11 (td, J=7.57, 1.46 Hz, 1H) 7.50 (d, J=8.30 Hz, 1H) 7.59 (s, 1H) 7.83 (d, J=7.81 Hz, 1H) 9.58 (d, J=7.81 Hz, 1H) 10.40 (s, 1H)

### Example 260

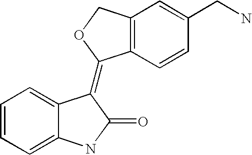

### 3-(5-Aminomethyl-3H-isobenzofuran-1-ylidene)-1,3-dihydro-indol-2-one

To a stirred solution of 1-(2-Oxo-1,2-dihydro-indol-3-ylidene)-1,3-dihydro-isobenzofuran-5-carbonitrile (200 mg, 0.73 mmol) in THF (20 ml) at reflux, 2M H₃B.SMe₂ solution in THF (2 ml, 4 mmol) was added drop wise under nitrogen. The reaction was heated at reflux for 2 hours, cooled with ice, and quenched with 2M HCl solution (9 ml). The mixture was heated at reflux for 30 minutes, and poured into 175 ml of water. The solution was basified with 5M NaOH and the resulting precipitates were filtered, washed with water, and dried in vacuo. Purification of the resulting crude product through silica gel chromatography with a gradient of MeOH in CHCl₃ gave the title compound as a yellow oil. Addition of 4M HCl/1,4-dioxane followed by evaporation of solvent gave the HCl salt as brown crystals (30 mg).

¹H NMR (HCl salt, 500 MHz, DMSO-d6) δ ppm 4.19 (q, J=5.70 Hz, 2H) 5.85 (s, 2H) 6.84 (d, J=7.32 Hz, 1H) 6.98 (td, J=7.57, 0.98 Hz, 1H) 7.13 (td, J=7.69, 1.22 Hz, 1H) 7.67 (d, J=8.30 Hz, 1H) 7.73 (s, 1H) 7.84 (d, J=7.81 Hz, 1H) 8.39 (br. s., 3H) 9.67 (d, J=8.30 Hz, 1H) 10.47 (s, 1H)

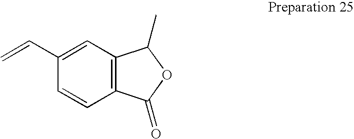

### Preparation of 3-methyl-5-vinyl-3H-isobenzofuran-1-one

A solution of trifluoromethanesulfonic acid 3-methyl-1-oxo-1,3-dihydro-isobenzofuran-5-yl ester (2.96 g, 10.0 mmol) in a mixture of isopropanol (67 ml) and H₂O (33 ml) at room temperature was degassed with argon. To this solution was added potassium vinyltrifluoroborate (1.47 g, 111.0 mmol) and 1,1′-bis(diphenylphosphino)-ferrocene-palladium(II)dichloride dichloromethane complex (163 mg, 0.20 mmol). Then while bubbling in argon and heating the solution at 70° C. for 2 minutes, triethylamine (4.2 ml, 30.0 mmol) was added over 1 minute. The argon addition was stopped after 2 minutes and the reaction heated at 70° C. for 1.25 hours. Upon cooling, 4% HCl aqueous solution was added until pH 7, and the solution was concentrated. The mixture was partitioned between EtOAc and 4% HCl, and the organic layer washed with H₂O, 0.3M NaOH, dilute HCl, brine, dried over Na₂SO₄ and evaporated to oil in orange color. The oil was passed through a plug of silica gel eluting with CHCl₃ gave the title compound as light orange oil (1.73 g, 99%).

¹H NMR (500 MHz, CDCl₃) δ ppm 1.65 (d, J=6.83 Hz, 3H) 5.47 (d, J=10.74 Hz, 1H) 5.55 (q, J=6.83 Hz, 1H) 5.92 (d, J=17.57 Hz, 1H) 6.81 (dd, J=17.57, 10.74 Hz, 1H) 7.42 (s, 1H) 7.54-7.57 (m, 1H) 7.84 (d, J=7.81 Hz, 1H).

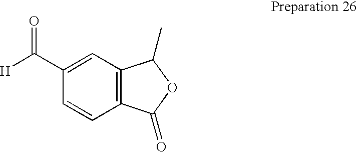

### Preparation of 3-methyl-1-oxo-1,3-dihydro-isobenzofuran-5-carbaldehyde

To a solution of 3-methyl-5-vinyl-3H-isobenzofuran-1-one (1.61 g, 9.3 mmol) in methanol (180 ml) at −78° C. was bubbled in ozone for 15 minutes giving a bluish-purple solution. The ozone addition was stopped, methyl sulfide (3.4 ml, 46.3 mmol) added, and after 5 minutes the cold bath removed to allow the reaction to warm to room temperature. The solution was evaporated, dissolved in EtOAc, and the organic layer washed with 4% HCl/brine mixture, brine, dried over anhydrous Na₂SO₄ and evaporated to light yellow oil. The oil was crystallized from EtOAc/hexane to give the title compound as an off-white solid (1.1 g, 67%).

¹H NMR (500 MHz, CDCl₃) δ ppm 1.70 (d, J=6.83 Hz, 3H) 5.66 (q, J=6.83 Hz, 1H) 7.98 (s, 1H) 8.03-8.09 (m, 2H) 10.18 (s, 1H).

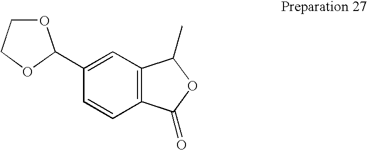

### Preparation of 5-[1,3]-dioxolan-2-yl-3-methyl-3H-isobenzofuran-1-one

A solution of 3-methyl-1-oxo-1,3-dihydro-isobenzofuran-5-carbaldehyde (1.49 g, 8.5 mmol), ethylene glycol (1.3 ml, 23.6 mmol), and catalytic p-toluenesulfonic acid in toluene (25 ml) was heated at reflux using a Dean-Stark trap for 50 minutes. The mixture was dissolved in EtOAc and the organic layer washed with saturated NaHCO₃, H₂O, and brine, dried over anhydrous Na₂SO₄, and evaporated to an off-white solid. The solid was triturated with EtOAc/hexane to give the title compound as a white solid (1.68 g, 90%).

¹H NMR (500 MHz, CDCl₃) δ ppm 1.65 (d, J=6.83 Hz, 3H) 4.08-4.17 (m, 4H) 5.57 (q, J=6.51 Hz, 1H) 5.91 (s, 1H) 7.57 (d, J=0.98 Hz, 1H) 7.63-7.65 (m, 1H) 7.90 (d, J=7.81 Hz, 1H).

### Example 261

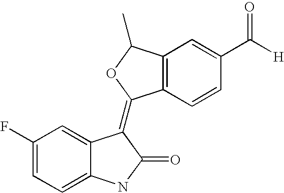

### 1-(5-Fluoro-2-oxo-1,2-dihydro-indol-3-ylidene)-3-methyl-1,3-dihydro-isobenzofuran-5-carbaldehyde

To a solution of 5-fluorooxindole (1.15 g, 7.6 mmol) in THF (30 ml) at 0° C. was added 1.0M LiHMDS/THF (16.0 ml) over 3 minutes. After the solution was allowed to warm to room temperature and then continuously stirred for 10 minutes, the solid, 5-[1,3]dioxolan-2-yl-3-methyl-3H-isobenzofuran-1-one (0.84 g, 3.8 mmol), was added in one portion. The reaction mixture was rapidly stirred at room temperature for 1 hour, and then quenched into 10% HCl aqueous solution (100 ml), followed by the addition of THF (20 ml). The mixture was gently warmed until a clear orange solution was obtained. The solution was stirred at room temperature for 1.3 hours, and then H₂O added. The resulting red precipitate was filtered and rinsed with H₂O. The solid was dissolved in EtOAc by warming, washed with 4% HCl, brine, dried over anhydrous Na₂SO₄, and evaporated to a solid. The solid was triturated with methanol and then recrystallized from THF to give the title compound as an orange solid (0.43 g, 37%).

¹H NMR (500 MHz, DMSO-d6) δ ppm 1.74 (d, J=6.83 Hz, 3H) 6.17 (q, J=6.83 Hz, 1H) 6.82 (dd, J=8.54, 4.64 Hz, 1H) 6.99 (ddd, J=9.64, 8.42, 2.44 Hz, 1H) 7.63 (dd, J=9.52, 2.68 Hz, 1H) 8.11 (dd, J=8.30, 1.46 Hz, 1H) 8.16 (s, 1H) 9.78 (d, J=8.30 Hz, 1H) 10.14 (s, 1H) 10.55 (s, 1H).

The following Examples 262-265 were prepared using the experiment procedure described in Example 261, but with the appropriate reagent, reaction conditions and reactant substitutions that will be readily realized by those of ordinary skill in this art without the exercise of undue experimentation.

### Example 262

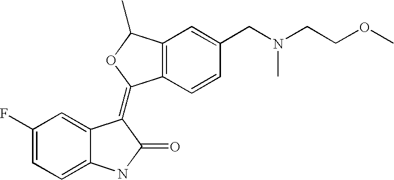

### 5-Fluoro-3-(5-{[(2-methoxy-ethyl)-methyl-amino]-methyl}-3-methyl-3H-isobenzofuran-1-ylidene)-1,3-dihydro-indol-2-one

¹H NMR (500 MHz, DMSO-d6) δ ppm 1.68 (d, J=6.35 Hz, 3H) 2.21 (s, 3H) 2.54-2.58 (m, 2H) 3.24 (s, 3H) 3.48 (t, J=6.10 Hz, 2H) 3.66 (s, 2H) 6.07 (q, J=6.35 Hz, 1H) 6.79 (dd, J=8.30, 4.88 Hz, 1H) 6.91-6.96 (m, 1H) 7.51 (dd, J=8.30, 0.98 Hz, 1H) 7.57-7.61 (m, 2H) 9.54 (d, J=7.81 Hz, 1H) 10.42 (s, 1H).

### Example 263

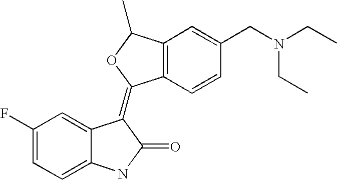

### 3-(5-Diethylaminomethyl-3-methyl-3H-isobenzofuran-1-ylidene)-5-fluoro-1,3-dihydro-indol-2-one

¹H NMR (500 MHz, DMSO-d6) δ ppm 1.00 (t, J=7.08 Hz, 6H) 1.68 (d, J=6.83 Hz, 3H) 2.47-2.52 (obscured q, 4H) 3.67 (s, 2H) 6.07 (q, J=6.67 Hz, 1H) 6.79 (dd, J=8.30, 4.88 Hz, 1H) 6.91-6.95 (m, 1H) 7.53 (d, J=8.79 Hz, 1H) 7.57-7.61 (m, 2H) 9.53 (d, J=8.30 Hz, 1H) 10.42 (s, 1H).

### Example 264

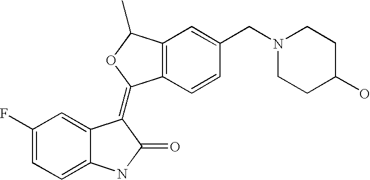

### 5-Fluoro-3-[5-(4-hydroxy-piperidin-1-ylmethyl)-3-methyl-3H-isobenzofuran-1-ylidene]-1,3-dihydro-indol-2-one

¹H NMR (500 MHz, DMSO-d6) δ ppm 1.37-1.46 (m, 2H) 1.66-1.75 (m, 5H) 2.04-2.12 (m, 2H) 2.68 (m, 2H) 3.43-3.50 (m, 1H) 3.52-3.57 (m, 1H) 3.58-3.63 (m, 1H) 4.56 (d, J=4.39 Hz, 1H) 6.07 (q, J=6.35 Hz, 1H) 6.79 (dd, J=8.30, 4.39 Hz, 1H) 6.90-6.96 (m, 1H) 7.50 (d, J=8.30 Hz, 1H) 7.56 (s, 1H) 7.59 (dd, J=9.52, 2.69 Hz, 1H) 9.54 (d, J=8.30 Hz, 1H) 10.42 (s, 1H).

### Example 265

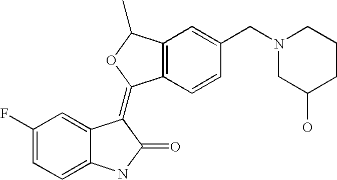

### 5-Fluoro-3-[5-(3-hydroxy-piperidin-1-ylmethyl)-3-methyl-3H-isobenzofuran-1-ylidene]-1,3-dihydro-indol-2-one

¹H NMR (500 MHz, DMSO-d6) δ ppm 1.04-1.12 (m, 1H) 1.40-1.50 (m, 1H) 1.60-1.66 (m, 1H) 1.69 (d, J=6.59 Hz, 3H) 1.73-1.83 (m, 2H) 1.88-1.96 (m, 1H) 2.62-2.70 (m, 1H) 2.76-2.84 (m, 1H) 3.45-3.53 (m, 1H) 3.52-3.69 (m, 2H) 4.60 (dd, J=7.14, 4.94 Hz, 1H) 6.07 (q, J=6.59 Hz, 1H) 6.79 (dd, J=8.42, 4.76 Hz, 1H) 6.91-6.96 (m, 1H) 7.51 (d, J=8.05 Hz, 1H) 7.57 (s, 1H) 7.59 (dd, J=9.52, 2.93 Hz, 1H) 9.54 (d, J=8.42 Hz, 1H) 10.42 (s, 1H).

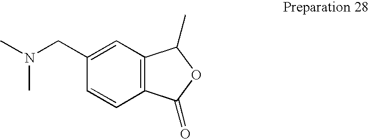

### Preparation of 5-Dimethylaminomethyl-3-methyl-3H-isobenzofuran-1-one

A solution of 3-methyl-1-oxo-1,3-dihydro-isobenzofuran-5-carbaldehyde (123 mg, 0.70 mmol), 2.0M dimethylamine/THF (0.49 ml), and sodium triacetoxyborohydride (178 mg, 0.84 mmol) in THF (4.0 ml) at room temperature was stirred for 16 hours. Then to push reaction to completion, added DMF (2.0 ml), 2 drops acetic acid, and 2.0M dimethylamine/THF (0.30 ml) and stirred reaction at room temperature for an additional 3 hours. The reaction was quenched into 4% HCl aqueous solution (20 ml) and then washed with EtOAc. The aqueous layer was basified with saturated NaHCO₃ and extracted with EtOAc. The combined EtOAc layers were washed with H₂O, brine, dried with anhydrous Na₂SO₄, and evaporated to the title compound as light yellow oil (98 mg, 68%).

¹H NMR (500 MHz, CDCl₃) δ ppm 1.64 (d, J=6.83 Hz, 3H) 2.28 (s, 6H) 3.51-3.58 (m, 2H) 5.54 (q, J=6.51 Hz, 1H) 7.44 (s, 1H) 7.46 (d, J=7.81 Hz, 1H) 7.83 (d, J=7.81 Hz, 1H).

### Example 266

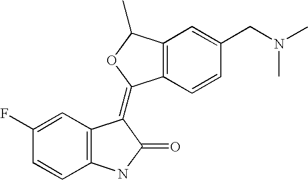

### 3-(5-Dimethylaminomethyl-3-methyl-3H-isobenzofuran-1-ylidene)-5-fluoro-1,3-dihydro-indol-2-one

To a solution of 5-fluorooxindole (140 mg, 0.93 mmol) in THF (3.5 ml) at 0° C. was added 1.0M LiHMDS/THF (1.9 ml) over 3 minutes. After the ice bath was removed, the solution was stirred for 10 minutes at room temperature, and then 5-dimethylaminomethyl-3-methyl-3H-isobenzofuran-1-one (95 mg, 0.46 mmol) in THF (0.8 ml) added. The reaction mixture was stirred at room temperature for 2.5 hours, then quenched with 4% HCl aqueous solution (30 ml) and stirred for 10 minutes. The aqueous layer was washed with EtOAc, basified with saturated NaHCO₃, extracted with EtOAc. The combined organic layers were washed with H₂O, brine, dried over anhydrous Na₂SO₄, and evaporated to a yellow solid. The solid was recrystallized from EtOAc/hexane to give 3-(5-dimethylaminomethyl-3-methyl-3H-isobenzofuran-1-ylidene)-5-fluoro-1,3-dihydro-indol-2-one as a yellow solid (95 mg, 61%).

¹H NMR (500 MHz, DMSO-d6) δ ppm 1.69 (d, J=6.35 Hz, 3H) 2.19 (s, 6H) 3.50-3.57 (m, 2H) 6.07 (q, J=6.83 Hz, 1H) 6.79 (dd, J=8.54, 4.64 Hz, 1H) 6.93 (ddd, J=9.64, 8.42, 2.44 Hz, 1H) 7.50 (dd, J=8.30, 0.98 Hz, 1H) 7.57 (s, 1H) 7.59 (dd, J=9.52, 2.68 Hz, 1H) 9.54 (d, J=8.30 Hz, 1H) 10.43 (s, 1H).

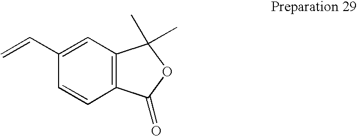

### Preparation of 3,3-dimethyl-5-vinyl-3H-isobenzofuran-1-one

Experimental Procedure Similar to Preparation 25

¹H NMR (500 MHz, CDCl₃) δ ppm 1.67 (s, 6H) 5.47 (d, J=11.23 Hz, 1H) 5.93 (d, J=17.57 Hz, 1H) 6.81 (dd, J=17.57, 10.74 Hz, 1H) 7.37 (s, 1H) 7.54 (dd, J=8.05, 1.22 Hz, 1H) 7.81 (d, J=7.81 Hz, 1H).

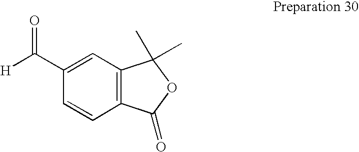

### Preparation of 3,3-dimethyl-1-oxo-1,3-dihydro-isobenzofuran-5-carbaldehyde

Experimental Procedure Similar to Preparation 25

¹H NMR (500 MHz, CDCl₃) δ ppm 1.72 (s, 6H) 7.94-7.94 (m, 1H) 8.01-8.06 (m, 2H) 10.17 (s, 1H).

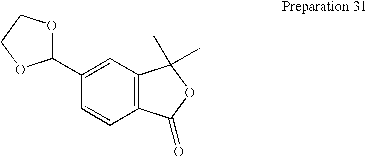

### Preparation of 5-[1,3]-dioxolan-2-yl-3,3-dimethyl-3H-isobenzofuran-1-one

Experimental Procedure Similar to Preparation 25

¹H NMR (500 MHz, CDCl₃) δ ppm 1.67 (s, 6H) 4.06-4.19 (m, 4H) 5.89 (s, 1H) 7.54 (d, J=0.98 Hz, 1H) 7.62 (dd, J=8.06, 1.22 Hz, 1H) 7.87 (d, J=7.81 Hz, 1H).

### Example 267

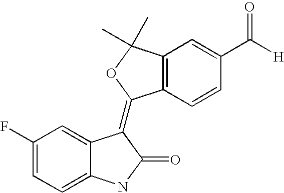

### 1-(5-Fluoro-2-oxo-1,2-dihydro-indol-3-ylidene)-3,3-dimethyl-1,3-dihydro-isobenzofuran-5-carbaldehyde

Experimental Procedure Similar to Preparation 25

¹H NMR (500 MHz, DMSO-d6) δ ppm 1.79 (s, 6H) 6.82 (dd, J=8.30, 4.39 Hz, 1H) 6.99 (ddd, J=9.64, 8.42, 2.93 Hz, 1H) 7.64 (dd, J=9.28, 2.44 Hz, 1H) 8.11 (dd, J=8.30, 1.46 Hz, 1H) 8.22 (d, J=1.46 Hz, 1H) 9.78 (d, J=8.30 Hz, 1H) 10.14 (s, 1H) 10.55 (s, 1H).

### Example 268

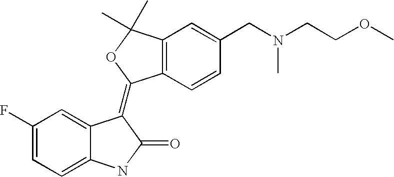

### 5-Fluoro-3-(5-{[(2-methoxy-ethyl)-methyl-amino]-methyl}-3,3-dimethyl-3H-isobenzofuran-1-ylidene)-1,3-dihydro-indol-2-one

A solution of 1-(5-fluoro-2-oxo-1,2-dihydro-indol-3-ylidene)-3,3-dimethyl-1,3-dihydro-isobenzofuran-5-carbaldehyde (75 mg, 0.23 mmol), 2-methoxy-n-methylethylamine (0.10 ml, 0.93 mmol), and sodium triacetoxyborohydride (59 mg, 0.28 mmol) in THF (3.0 ml) at room temperature was stirred for 2.5 hours. The reaction was partitioned between saturated NaHCO₃ and EtOAc. The EtOAc layer was washed with H₂O, brine, dried over anhydrous Na₂SO₄, and evaporated to give an orange solid. The solid was chromatographed eluting with gradient 3% to 6% methanol in EtOAc to give the title compound as a yellow solid (65 mg, 70%).

¹H NMR (500 MHz, CDCl₃) δ ppm 1.74 (s, 6H) 2.33 (s, 3H) 2.63 (t, J=5.61 Hz, 2H) 3.35 (s, 3H) 3.53 (t, J=5.61 Hz, 2H) 3.69 (s, 2H) 6.76-6.78 (m, 1H) 6.85 (ddd, J=9.40, 8.42, 2.68 Hz, 1H) 7.40 (d, J=0.98 Hz, 1H) 7.46 (dd, J=8.30, 1.46 Hz, 1H) 7.70 (dd, J=9.52, 2.69 Hz, 1H) 7.90 (s, 1H) 9.58 (d, J=8.30 Hz, 1H).

### Example 269

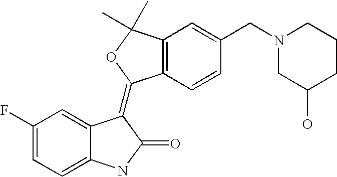

### 5-Fluoro-3-[5-(3-hydroxy-piperidin-1-ylmethyl)-3,3-dimethyl-3H-isobenzofuran-1-ylidene]-1,3-dihydro-indol-2-one

Procedure Similar to Example 268.

¹H NMR (500 MHz, DMSO-d6) δ ppm 1.05-1.13 (m, 1H) 1.41-1.50 (m, 1H) 1.61-1.66 (m, 1H) 1.73 (s, 6H) 1.74-1.82 (m, 2H) 1.89-1.95 (m, 1H) 2.65 (d, J=10.74 Hz, 1H) 2.79 (dd, J=10.25, 3.42 Hz, 1H) 3.50 (tq, J=9.49, 4.58 Hz, 1H) 3.54-3.58 (m, 1H) 3.61-3.66 (m, 1H) 4.60 (d, J=4.88 Hz, 1H) 6.79 (dd, J=8.54, 4.64 Hz, 1H) 6.93 (ddd, J=9.76, 8.54, 2.68 Hz, 1H) 7.50 (dd, J=8.30, 0.98 Hz, 1H) 7.58-7.61 (m, 2H) 9.52 (d, J=8.30 Hz, 1H) 10.41 (s, 1H).

### Example 270

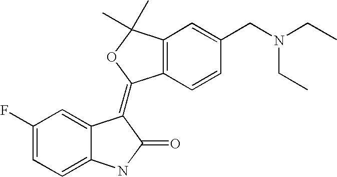

### 3-(5-Diethylaminomethyl-3,3-dimethyl-3H-isobenzofuran-1-ylidene)-5-fluoro-1,3-dihydro-indol-2-one

A solution of 1-(5-fluoro-2-oxo-1,2-dihydro-indol-3-ylidene)-3,3-dimethyl-1,3-dihydro-isobenzofuran-5-carbaldehyde (178 mg, 0.55 mmol), diethylamine (0.14 ml, 1.38 mmol), acetic acid (9.4 μl, 0.17 mmol), and triacetoxyborohydride (140 mg, 0.66 mmol) in DMF (3.0 ml) was stirred at room temperature for 21 hours. The reaction was dissolved in EtOAc and washed with saturated NaHCO₃, H₂O, brine, dried over anhydrous Na₂SO₄, and evaporated to an orange solid. The solid was partitioned between 4% HCl and EtOAc, and the aqueous layer was basified with saturated NaHCO₃, extracted with EtOAc. The combined organic layers were washed with H₂O, brine, dried over anhydrous Na₂SO₄, and evaporated to a yellow solid. The solid was chromatographed eluting with gradient 4% to 6% methanol in CHCl₃ to the title compound as a yellow solid (46 mg, 22%).

¹H NMR (500 MHz, CDCl₃) δ ppm 1.06 (t, J=7.32 Hz, 6H) 1.74 (s, 6H) 2.56 (q, J=7.00 Hz, 4H) 3.69 (s, 2H) 6.75-6.79 (m, 1H) 6.85 (td, J=8.91, 2.69 Hz, 1H) 7.38 (s, 1H) 7.48 (d, J=8.30 Hz, 1H) 7.70 (dd, J=9.76, 2.44 Hz, 1H) 7.88 (s, 1H) 9.57 (d, J=8.30 Hz, 1H).

### Example 271

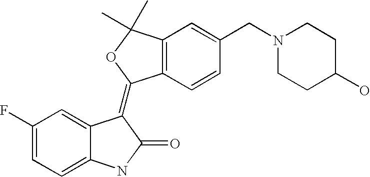

### 5-Fluoro-3-[5-(4-hydroxy-piperidin-1-ylmethyl)-3,3-dimethyl-3H-isobenzofuran-1-ylidene]-1,3-dihydro-indol-2-one

A solution of 1-(5-fluoro-2-oxo-1,2-dihydro-indol-3-ylidene)-3,3-dimethyl-1,3-dihydro-isobenzofuran-5-carbaldehyde (178 mg, 0.55 mmol), 4-hydroxypiperidine (139 mg, 1.38 mmol), acetic acid (9.4 μl, 0.17 mmol), and triacetoxyborohydride (140 mg, 0.66 mmol) in DMF (3.0 ml) was stirred at room temperature for 20 hours. The reaction was quenched into NaHCO₃ solution (25 ml) and the yellow precipitate was filtered and rinsed with H₂O. The solid was dissolved in EtOAc and washed with H₂O, brine, dried over anhydrous Na₂SO₄, and evaporated to orange foam. The solid was chromatographed eluting with gradient 4% to 6% methanol in CHCl₃ to give the title compound as a yellow solid (44 mg, 20%). Also isolated was 3-(5-dimethylaminomethyl-3,3-dimethyl-3H-isobenzofuran-1-ylidene)-5-fluoro-1,3-dihydro-indol-2-one as a yellow solid (13 mg).

¹H NMR (500 MHz, DMSO-d6) δ ppm 1.39-1.46 (m, 2H) 1.70-1.74 (m, 8H) 2.08 (t, J=9.76 Hz, 2H) 2.66-2.71 (m, 2H) 3.47 (td, J=8.67, 4.64 Hz, 1H) 3.58 (s, 2H) 4.56 (d, J=3.91 Hz, 1H) 6.79 (dd, J=8.54, 4.64 Hz, 1H) 6.93 (ddd, J=9.64, 8.42, 2.93 Hz, 1H) 7.50 (dd, J=8.30, 1.46 Hz, 1H) 7.57 (s, 1H) 7.59 (dd, J=9.76, 2.93 Hz, 1H) 9.51 (d, J=8.30 Hz, 1H) 10.41 (s, 1H).

Another yellow solid was isolated as Example 272.

### Example 272

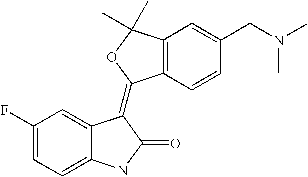

### 3-(5-Dimethylaminomethyl-3,3-dimethyl-3H-isobenzofuran-1-ylidene)-5-fluoro-1,3-dihydro-indol-2-one

¹H NMR (500 MHz, CDCl₃) δ ppm 1.74 (s, 7H) 2.29 (s, 7H) 6.77-6.79 (m, 1H) 6.82-6.87 (m, 1H) 7.36 (d, J=0.98 Hz, 1H) 7.45 (dd, J=8.30, 1.46 Hz, 1H) 7.69 (dd, J=9.52, 2.68 Hz, 1H) 8.31 (s, 1H) 9.61 (d, J=7.81 Hz, 1H).

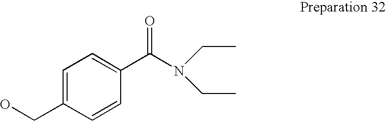

### Preparation of N,N-diethyl-4-hydroxymethyl-benzamide

To a solution containing 4-(hydroxymethyl)benzoic acid (5.51 g, 36.2 mmol), diethylamine (8.2 ml, 79.6 mmol) and 1-hydroxybenzotriazole hydrate (5.14 g, 38.0 mmol) in DMF (60 ml) at room temperature was added 1-[3-(dimethylamino)propyl]-3-ethylcarbodiimide hydrochloride (7.63 g, 39.8 mmol). After 18 hours the reaction was concentrated and then dissolved in EtOAc. The EtOAc solution was washed with dilute aqueous HCl, H₂O/brine mixture, saturated aqueous NaHCO₃, brine and then dried over anhydrous Na₂SO₄. Concentration of the EtOAc solution gave a precipitate which was filtered to give the title compound as a white solid (6.38 g, 85%).

¹H NMR (500 MHz, CDCl₃) δ ppm 1.10 (broad s, 3H) 1.24 (broad s, 3H) 2.51 (t, J=6.10 Hz, 1H) 3.24 (broad s, 2H) 3.54 (broad s, 2H) 4.68 (d, J=5.86 Hz, 2H) 7.30-7.34 (m, 4H).

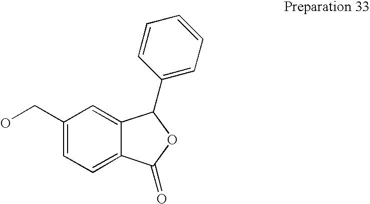

### Preparation of 5-hydroxymethyl-3-phenyl-3H-isobenzofuran-1-one

To a solution of N,N,N′,N′-tetramethylethylenediamine (0.217 ml, 1.4 mmol) in THF (10.0 ml) at −78° C. was added 1.4M s-BuLi/cyclohexane solution (1.5 ml, 2.0 mmol). Upon stirring for 10 minutes, N,N-diethyl-4-hydroxymethyl-benzamide (142 mg, 0.68 mmol) in THF (11.0 ml) was added over 6 minutes. After stirring 25 minutes, benzaldehyde (0.139 ml, 1.37 mmol) was added, and the solution continuously stirred 45 minutes. The reaction was allowed to warm to room temperature. Then 10% HCl aqueous solution (10 ml) was added and the mixture stirred for 1.5 hour. The solution was extracted with EtOAc, and the combined organic layers were washed with H₂O, brine, dried with anhydrous Na₂SO₄, and evaporated to light yellow oil. The oil was crystallized from EtOAc/hexane to give the title compound as a white solid (104 mg, 63%).

¹H NMR (500 MHz, CDCl₃) δ ppm 1.95 (broad s, 1H) 4.80 (s, 2H) 6.38 (s, 1H) 7.26-7.29 (m, 2H) 7.35-7.40 (m, 4H) 7.51-7.53 (m, 1H) 7.93 (d, J=7.81 Hz, 1H).

### Example 273

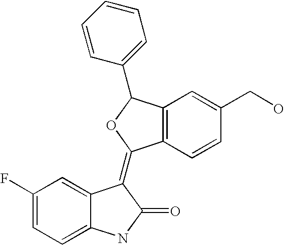

### 5-Fluoro-3-(5-hydroxymethyl-3-phenyl-3H-isobenzofuran-1-ylidene)-1,3-dihydro-indol-2-one

To a solution of 5-fluorooxindole (48 mg, 0.32 mmol) in THF (2.0 ml) at 0° C. was added 1.0M LiHMDS/THF (0.95 ml) over 30 seconds. The mixture was stirred for 2 minutes at 0° C., and then the ice bath was removed. After 5 minutes, 5-hydroxymethyl-3-phenyl-3H-isobenzofuran-1-one (53 mg, 0.22 mmol) in THF (0.5 ml) was added and the reaction mixture stirred at room temperature for 3.5 hours. The reaction was quenched with 10% HCl aqueous solution and then warmed several minutes. The mixture was extracted with EtOAc and the organic layer washed with H₂O, brine, dried over anhydrous Na₂SO₄, and evaporated to a yellow solid film. The solid was triturated with 30% EtOAc in hexane and then with MeOH to give the title compound as a yellow solid (11 mg, 14%).

¹H NMR (500 MHz, DMSO-d6) δ ppm 4.61 (d, J=5.86 Hz, 2H) 5.41-5.44 (m, 1H) 6.81 (dd, J=8.54, 4.64 Hz, 1H) 6.91-6.96 (m, 1H) 7.08 (s, 1H) 7.38 (did, J=6.35, 1.46 Hz, 3H) 7.41-7.47 (m, 4H) 7.53 (did, J=8.30, 0.98 Hz, 1H) 9.60 (d, J=8.30 Hz, 1H) 10.52 (s, 1H).

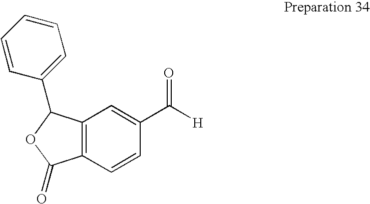

### Preparation of 1-oxo-3-phenyl-1,3-dihydro-isobenzofuran-5-carbaldehyde

To a solution of 5-hydroxymethyl-3-phenyl-3H-isobenzofuran-1-one (104 mg, 0.43 mmol) in dichloromethane at room temperature was added pyridinium chlorochromate (187 mg, 0.87 mmol) which had been absorbed to silica gel (261 mg) by grinding together. After 1.25 hours diethyl ether (4 ml) was added, the mixture filtered through celite plug, and rinsed with diethyl ether (20 ml). The solution was evaporated, dissolved in CHCl₃, and filtered through a plug of silica gel eluting with CHCl₃ to the title compound as a light greenish-white solid (98 mg, 95%).

¹H NMR (500 MHz, CDCl₃) δ ppm 6.49 (s, 1H) 7.28-7.31 (m, 2H) 7.40-7.42 (m, 3H) 7.85 (s, 1H) 8.06-8.09 (m, 1H) 8.12-8.14 (m, 1H) 10.11 (s, 1H).

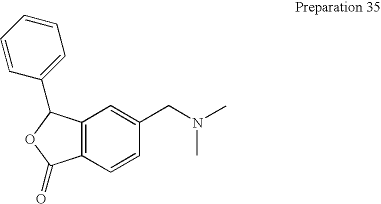

### Preparation of 5-dimethylaminomethyl-3-phenyl-3H-isobenzofuran-1-one

To a solution of 1-oxo-3-phenyl-1,3-dihydro-isobenzofuran-5-carbaldehyde (93 mg, 0.39 mmol) in DMF (2.5 ml) at room temperature was added acetic acid (0.01 ml, 0.17 mmol), 2.0M dimethylamine/THF (0.49 ml), and sodium triacetoxyborohydride (99 mg, 0.47 mmol). After 22 hours 10% HCl aqueous solution (10 ml) was added. The solution was diluted with H₂O and then washed with EtOAc. The aqueous layer was then basified with saturated aqueous NaHCO₃, and extracted with EtOAc. The combined organic layers were washed with H₂O, brine, dried over anhydrous Na₂SO₄, and evaporated to an off-white solid. The solid was triturated with hexane/EtOAc to give the title compound as a white solid (59 mg, 57%).

¹H NMR (500 MHz, CDCl₃) δ ppm 2.24 (s, 6H) 3.43-3.56 (m, 2H) 6.38 (s, 1H) 7.25-7.41 (m, 6H) 7.50 (d, J=7.81 Hz, 1H) 7.90 (d, J=7.81 Hz, 1H).

### Example 274

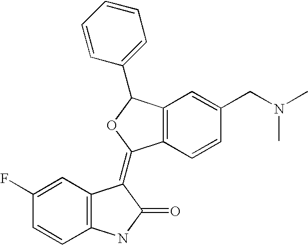

### 3-(5-Dimethylaminomethyl-3-phenyl-3H-isobenzofuran-1-ylidene)-5-fluoro-1,3-dihydro-indol-2-one

Experimental procedure similar to Example 266.

¹H NMR (500 MHz, DMSO-d6) δ ppm 2.13 (s, 6H) 3.45-3.51 (m, 2H) 6.81 (did, J=8.30, 4.88 Hz, 1H) 6.94 (ddd, J=9.64, 8.42, 2.93 Hz, 1H) 7.08 (s, 1H) 7.34 (s, 1H) 7.37-7.40 (m, 2H) 7.41-7.47 (m, 4H) 7.53 (did, J=8.30, 0.98 Hz, 1H) 9.60 (d, J=8.30 Hz, 1H) 10.52 (s, 1H).

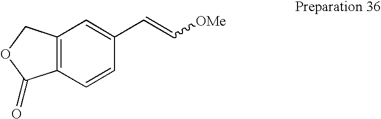

### Preparation of 5-(2-methoxy-vinyl)-3H-isobenzofuran-1-one

To a cooled suspension of (methoxymethyl)triphenylphosphonium chloride (1.58 g, 4.62 mmol) in anhydrous THF (15 ml) at 0° C. was slowly added 1M tertBuOK/tertBuOH solution (4.3 ml, 4.3 mmol) under nitrogen over 3 minutes. After the mixture was stirred at room temp for 30 minutes and then cooled back to 0° C., 5-formylphthalide (500 mg, 3.08 mmol) was added. The resulting mixture was stirred at room temperature for 5 hours and quenched with saturated ammonium chloride solution (20 ml) at 0° C. The aqueous layer was extracted with ethyl acetate (2×75 ml), and the organic layers were combined, washed with saturated NaCl solution (2×50 ml), dried over Na₂SO₄. Purification of the residue through silica gel chromatography with elution of 20% EtOAc/hexanes gave the title compound as a white solid (378 mg, 65%) in a mixture of cis and trans isomers (˜1:1 ratio).

### Example 275

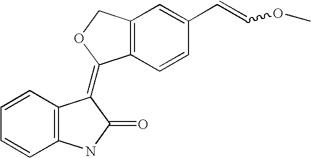

### 3-[5-(2-Methoxy-vinyl)-3H-isobenzofuran-1-ylidene]-1,3-dihydro-indol-2-one

To a stirred solution of oxindole (224 mg, 1.68 mmol) in anhydrous THF (10 ml) under nitrogen was added 1.0M LiHMDS/THF solution (3.4 ml, 3.4 mmol). The mixture was stirred at room temperature for 10 minutes, and then 5-(2-methoxy-vinyl)-3H-isobenzofuran-1-one (160 mg, 0.84 mmol) was added as one portion. After continuously stirring at room temperature for 2 hours, the mixture was poured into 10 ml of 2M HCl solution. The mixture was heated at 45° C. for 90 minutes, and poured into 125 ml of water. The solid was filtered, washed with water, and dried under vacuum to give the title compound as a yellow solid (180 mg, 70%) in a mixture of cis and trans isomers (˜1:1 ratio). MS (ES+): 306.1073 (MH⁺), 328.0874 (M+Na⁺)

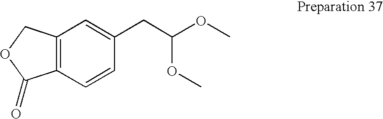

### Preparation of 5-(2,2-dimethoxy-ethyl)-3H-isobenzofuran-1-one

To a stirred solution of 5-(2-methoxy-vinyl)-3H-isobenzofuran-1-one (120 mg, 0.63 mmol) in 10 ml of anhydrous methanol was added 3 drops of concentrated sulfuric acid. The mixture was heated at reflux for 5 hours, cooled and diluted with ethyl acetate (100 ml). The EtOAc solution was washed with saturated NaHCO₃ solution (2×75 ml), dried over Na₂SO₄. Purification of the concentrated filtrate through silica gel column by elution with 10-20% ethyl acetate/hexanes gave the title compound as a clear oil (117 mg, 84%).

¹H NMR (500 MHz, CDCl₃) δ ppm 3.06 (d, J=5.37 Hz, 2H) 3.38 (s, 6H) 4.58 (t, J=5.37 Hz, 1H) 5.31 (s, 2H) 7.38 (s, 1H) 7.42 (d, J=7.81 Hz, 1H) 7.87 (d, J=7.81 Hz, 1H)

### Example 276

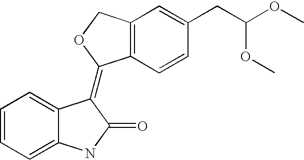

### 3-[5-(2,2-Dimethoxy-ethyl)-3H-isobenzofuran-1-ylidene]-1,3-dihydro-indol-2-one

To a stirred solution of oxindole (240 mg, 1.80 mmol) in anhydrous THF (7 ml) under nitrogen was added 1.0M LiHMDS/THF solution (3.6 ml, 3.6 mmol). After the mixture was stirred at room temperature for 10 minutes, 5-(2,2-dimethoxy-ethyl)-3H-isobenzofuran-1-one (200 mg, 0.90 mmol) was added as one portion. After continuously stirring at room temperature for 1.5 hours, the mixture was poured into 10 ml of 1M sulfuric acid. The mixture was heated at 45° C. for 30 minutes, and poured into 200 ml of water. The solid was filtered, washed with water, and dried in vacuum to give the title compound as a yellow solid (210 mg, 69%).

¹H NMR (500 MHz, DMSO-d6) δ ppm 3.00 (d, J=5.37 Hz, 2H) 3.27 (s, 6H) 4.64 (t, J=5.86 Hz, 1H) 5.79 (s, 2H) 6.82 (d, J=7.81 Hz, 1H) 6.95 (t, J=7.57 Hz, 1H) 7.10 (t, J=7.08 Hz, 1H) 7.45 (d, J=9.28 Hz, 1H) 7.52 (s, 1H) 7.83 (d, J=7.81 Hz, 1H) 9.55 (d, J=8.30 Hz, 1H) 10.40 (s, 1H)

The following Examples 277-278 were prepared using the experiment procedure described in Example 276, but with the appropriate reagent, reaction conditions and reactant substitutions that will be readily realized by those of ordinary skill in this art without the exercise of undue experimentation.

### Example 277

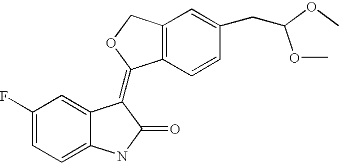

### 3-[5-(2,2-Dimethoxy-ethyl)-3H-isobenzofuran-1-ylidene]-5-fluoro-1,3-dihydro-indol-2-one

### Example 278

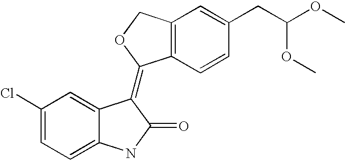

### 5-Chloro-3-[5-(2,2-dimethoxy-ethyl)-3H-isobenzofuran-1-ylidene]-1,3-dihydro-indol-2-one

¹H NMR (500 MHz, DMSO-d6) δ ppm 3.01 (d, J=5.86 Hz, 2H) 3.27 (s, 6H) 4.64 (t, J=5.37 Hz, 1H) 5.83 (s, 2H) 6.83 (d, J=8.30 Hz, 1H) 7.14 (did, J=8.30, 2.44 Hz, 1H) 7.47 (d, J=8.30 Hz, 1H) 7.55 (s, 1H) 7.79 (d, J=2.44 Hz, 1H) 9.53 (d, J=8.30 Hz, 1H) 10.54 (s, 1H)

### Example 279

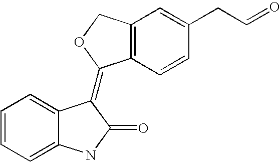

### [1-(2-Oxo-1,2-dihydro-indol-3-ylidene)-1,3-dihydro-isobenzofuran-5-yl]-acetaldehyde

To a stirred solution of 3-[5-(2,2-dimethoxy-ethyl)-3H-isobenzofuran-1-ylidene]-1,3-dihydro-indol-2-one (155 mg, 0.46 mmol) in 10 ml of THF, was added 2 ml of 2.5M sulfuric acid. The mixture was heated at 60° C. for 30 minutes, cooled to room temp., and poured into 150 ml of water with stirring. The mixture was stirred for 2 hours, filtered, washed with water, and dried in vacuum. Removal of the solvent gave the title compound as a yellow solid (132 mg, 100%). MS (ES+): 292.1006 (MH⁺)

### Example 280

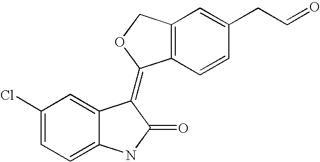

### [1-(5-Chloro-2-oxo-1,2-dihydro-indol-3-ylidene)-1,3-dihydro-isobenzofuran-5-yl]-acetaldehyde

Experimental procedure similar to Example 279.

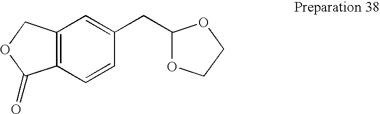

### Preparation of 5-[1,3]dioxolan-2-ylmethyl-3H-isobenzofuran-1-one

Experimental procedure similar to Preparation 24.

### Example 281

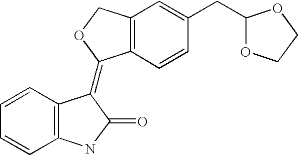

### 3-(5-[1,3]Dioxolan-2-ylmethyl-3H-isobenzofuran-1-ylidene)-1,3-dihydro-indol-2-one

To a stirred solution of oxindole (72 mg, 0.54 mmol) in anhydrous THF (5 ml) under nitrogen was added 1.0M LiHMDS/THF solution (1.1 ml, 1.1 mmol). The mixture was stirred at room temperature for 10 minutes, and then 5-[1,3]dioxolan-2-ylmethyl-3H-isobenzofuran-1-one (60 mg, 0.27 mmol) was added. After continuously stirring at room temperature for 1 hour, the mixture was poured into 1M HCl aqueous solution (10 ml) and heated at 50° C. for 1 hour. The mixture was cooled to room temperature, and then poured into water (250 ml) with vigorous stirring. The precipitates were separated, rinsed with water, and dried in vacuum to give the title compound (76 mg, 84%).

¹H NMR (500 MHz, DMSO-d6) δ ppm 3.04 (d, J=4.88 Hz, 2H) 3.76-3.80 (m, 2H) 3.87-3.92 (m, 2H) 5.07 (t, J=4.88 Hz, 1H) 5.79 (s, 2H) 6.82 (d, J=7.81 Hz, 1H) 6.95 (td, J=7.57, 0.98 Hz, 1H) 7.10 (t, J=8.30 Hz, 1H) 7.46 (d, J=8.79 Hz, 1H) 7.54 (s, 1H) 7.83 (d, J=7.81 Hz, 1H) 9.55 (d, J=7.81 Hz, 1H) 10.40 (s, 1H)

### Example 282

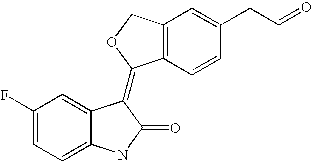

### [1-(5-Fluoro-2-oxo-1,2-dihydro-indol-3-ylidene)-1,3-dihydro-isobenzofuran-5-yl]-acetaldehyde

### Example 283

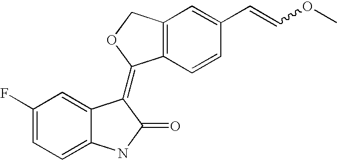

### 5-Fluoro-3-[5-(2-methoxy-vinyl)-3H-isobenzofuran-1-ylidene]-1,3-dihydro-indol-2-one

Experimental procedure similar to Example 275

MS (ES+): 324.0990 (MH⁺), 346.0819 (M+Na⁺)

### Example 284

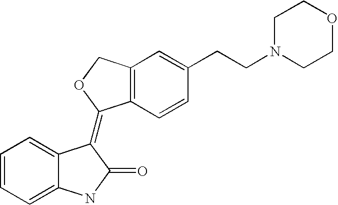

### 3-[5-(2-Morpholin-4-yl-ethyl)-3H-isobenzofuran-1-ylidene]-1,3-dihydro-indol-2-one

To a stirred suspension of [1-(2-oxo-1,2-dihydro-indol-3-ylidene)-1,3-dihydro-isobenzofuran-5-yl]-acetaldehyde (22 mg, 0.076 mmol), morpholine (10 mg, 0.11 mmol) and acetic acid (10 mg, 0.17 mmol) in 2 ml of methanol was added sodium cyanoborohydride (10 mg, 0.16 mmol). The mixture was stirred for 30 minutes, and poured into 70 ml of water with stirring. The mixture was basified with saturated NaHCO₃ solution and then stirred at room temperature for 30 minutes. The precipitate was filtered, washed with water, and dried in vacuum to the title compound as a yellow solid (20 mg, 74%).

¹H NMR (500 MHz, DMSO-d6) δ ppm 2.44 (s, 4H) 2.58 (t, J=7.32 Hz, 2H) 2.89 (t, J=7.32 Hz, 2H) 3.58 (t, J=4.39 Hz, 4H) 5.78 (s, 2H) 6.82 (d, J=7.81 Hz, 1H) 6.95 (t, J=7.08 Hz, 1H) 7.10 (t, J=7.08 Hz, 1H) 7.45 (d, J=8.30 Hz, 1H) 7.52 (s, 1H) 7.83 (d, J=7.32 Hz, 1H) 9.55 (d, J=7.81 Hz, 1H) 10.39 (s, 1H)

The following Examples 285-422 were prepared using the experiment procedure described in Example 284, but with the appropriate reagent, reaction conditions and reactant substitutions that will be readily realized by those of ordinary skill in this art without the exercise of undue experimentation.

### Example 285

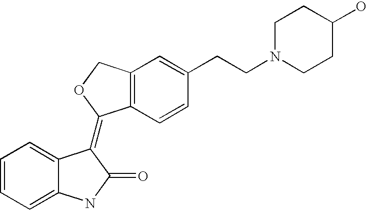

### 3-{5-[2-(4-Hydroxy-piperidin-1-yl)-ethyl]-3H-isobenzofuran-1-ylidene}-1,3-dihydro-indol-2-one

¹H NMR (500 MHz, DMSO-d6) δ ppm 1.33-1.42 (m, 2H) 1.68-1.74 (m, 2H) 2.08 (t, J=10.50 Hz, 2H) 2.55 (t, J=7.32 Hz, 2H) 2.73-2.81 (m, 2H) 2.86 (t, J=7.32 Hz, 2H) 3.39-3.50 (m, 1H) 4.53 (d, J=4.39 Hz, 1H) 5.77 (s, 2H) 6.82 (d, J=7.81 Hz, 1H) 6.95 (td, J=7.57, 0.98 Hz, 1H) 7.10 (td, J=7.69, 1.22 Hz, 1H) 7.43 (d, J=8.30 Hz, 1H) 7.50 (s, 1H) 7.82 (d, J=7.81 Hz, 1H) 9.54 (d, J=8.30 Hz, 1H) 10.39 (s, 1H)

### Example 286

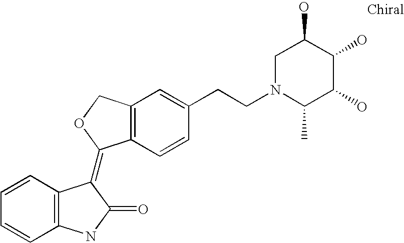

### 3-{5-[2-(3,4,5-Trihydroxy-2-methyl-piperidin-1-yl)-ethyl]-3H-isobenzofuran-1-ylidene}-1,3-dihydro-indol-2-one

¹H NMR (500 MHz, DMSO-d6) δ ppm 1.10 (s, 3H) 2.12 (br. s., 1H) 2.42 (br. s., 1H) 2.69 (br. s., 1H) 2.80 (br. s., 3H) 2.88-2.96 (m, 1H) 3.01-3.12 (m, 1H) 3.45 (br s, 1H) 3.59 (br. s., 1H) 3.81 (br s, 1H) 4.41 (br s, 1H) 4.57 (br s, 1H) 5.77 (s, 2H) 6.82 (d, J=7.32 Hz, 1H) 6.95 (t, J=7.57 Hz, 1H) 7.10 (t, J=7.08 Hz, 1H) 7.43 (d, J=6.83 Hz, 1H) 7.51 (s, 1H) 7.82 (d, J=7.32 Hz, 1H) 9.55 (d, J=7.81 Hz, 1H) 10.39 (s, 1H)

### Example 287

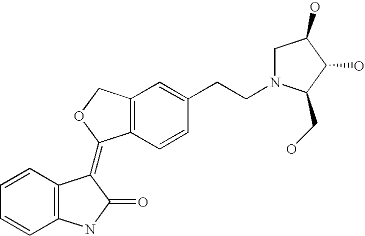

### 3-{5-[2-(3,4-Dihydroxy-2-hydroxymethyl-pyrrolidin-1-yl)-ethyl]-3H-isobenzofuran-1-ylidene}-1,3-dihydro-indol-2-one

### Example 288

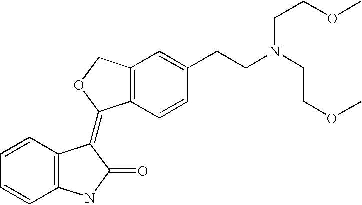

### 3-(5-{2-[Bis-(2-methoxy-ethyl)-amino]-ethyl}-3H-isobenzofuran-1-ylidene)-1,3-dihydro-indol-2-one

¹H NMR (500 MHz, DMSO-d6) δ ppm 2.71 (br. s., 4H) 2.81 (d, J=8.30 Hz, 4H) 3.23 (s, 6H) 3.38 (t, J=5.61 Hz, 4H) 5.78 (s, 2H) 6.83 (d, J=7.81 Hz, 1H) 6.96 (td, J=7.69, 1.22 Hz, 1H) 7.10 (td, J=7.69, 1.22 Hz, 1H) 7.43 (d, J=7.81 Hz, 1H) 7.51 (s, 1H) 7.83 (d, J=7.81 Hz, 1H) 9.54 (d, J=8.30 Hz, 1H) 10.39 (s, 1H)

### Example 289

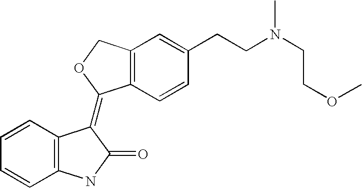

### 3-(5-{2-[(2-Methoxy-ethyl)-methyl-amino]-ethyl}-3H-isobenzofuran-1-ylidene)-1,3-dihydro-indol-2-one

¹H NMR (500 MHz, DMSO-d6) δ ppm 2.26 (s, 3H) 2.56 (t, J=5.86 Hz, 2H) 2.66 (t, J=7.32 Hz, 2H) 2.85 (t, J=7.32 Hz, 2H) 3.23 (s, 3H) 3.40 (t, J=5.86 Hz, 2H) 5.77 (s, 2H) 6.82 (d, J=7.81 Hz, 1H) 6.95 (td, J=7.57, 0.98 Hz, 1H) 7.10 (td, J=7.57, 1.46 Hz, 1H) 7.43 (d, J=8.30 Hz, 1H) 7.51 (s, 1H) 7.82 (d, J=7.32 Hz, 1H) 9.54 (d, J=8.30 Hz, 1H) 10.39 (s, 1H)

### Example 290

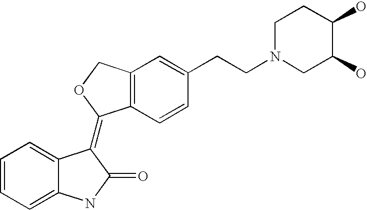

### cis-3-{5-[2-(3,4-Dihydroxy-piperidin-1-yl)-ethyl]-3H-isobenzofuran-1-ylidene}-1,3-dihydro-indol-2-one

¹H NMR (500 MHz, DMSO-d6) δ ppm 1.48-1.59 (m, 1H) 1.61-1.71 (m, 1H) 2.38 (br. s., 3H) 2.45-2.49 (m, 1H) 2.55-2.62 (m, 2H) 2.87 (t, J=7.57 Hz, 2H) 3.45-3.53 (m, 1H) 3.64 (br. s., 1H) 4.23 (d, J=3.91 Hz, 1H) 4.30 (d, J=5.37 Hz, 1H) 5.77 (s, 2H) 6.82 (d, J=7.32 Hz, 1H) 6.95 (td, J=7.57, 0.98 Hz, 1H) 7.10 (td, J=7.57, 0.98 Hz, 1H) 7.44 (d, J=8.30 Hz, 1H) 7.51 (s, 1H) 7.83 (d, J=7.32 Hz, 1H) 9.54 (d, J=8.30 Hz, 1H) 10.39 (s, 1H)

### Example 291

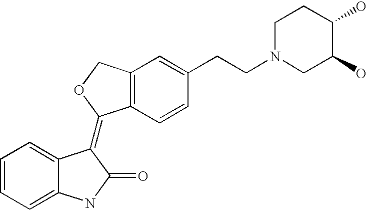

### trans-3-{5-[2-(3,4-Dihydroxy-piperidin-1-yl)-ethyl]-3H-isobenzofuran-1-ylidene}-1,3-dihydro-indol-2-one

¹H NMR (500 MHz, DMSO-d6) δ ppm 1.29-1.41 (m, 1H) 1.71-1.77 (m, 1H) 1.82 (t, J=10.01 Hz, 1H) 1.94-2.03 (m, 1H) 2.54-2.61 (m, 2H) 2.76-2.94 (m, 4H) 3.07-3.16 (m, 1H) 3.16-3.26 (m, 1H) 4.64 (d, J=4.39 Hz, 1H) 4.69 (d, J=4.39 Hz, 1H) 5.77 (s, 2H) 6.82 (d, J=7.81 Hz, 1H) 6.95 (td, J=7.69, 1.22 Hz, 1H) 7.10 (td, J=7.69, 1.22 Hz, 1H) 7.43 (d, J=8.30 Hz, 1H) 7.51 (s, 1H) 7.83 (d, J=7.81 Hz, 1H) 9.54 (d, J=8.30 Hz, 1H) 10.39 (s, 1H)

| Example | | | Molecular | Table |
|---|---|---|---|---|
| Number | Chemical Structure | Chemical Name | Weight | Number |
| 292 | 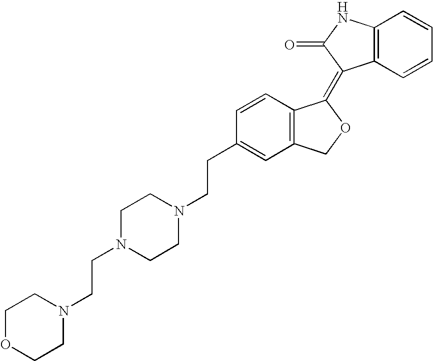 | 3-(5-{2-[4-(2-Morpholin- 4-yl-ethyl)-piperazin-1- yl]-ethyl}-3H- isobenzofuran-1- ylidene)-1,3-dihydro- indol-2-one | 474.59 | 8 |
| 293 | 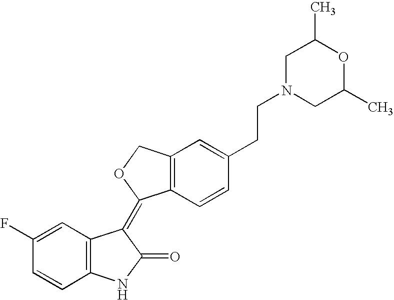 | 3-{5-[2-(2,6-Dimethyl- morpholin-4-yl)-ethyl]- 3H-isobenzofuran-1- ylidene}-5-fluoro-1,3- dihydro-indol-2-one | 408.47 | 8 |
| 294 | 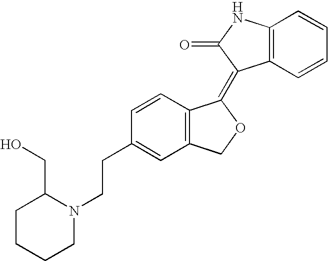 | 3-{5-[2-(2- Hydroxymethyl- piperidin-1-yl)-ethyl]-3H- isobenzofuran-1- ylidene}-1,3-dihydro- indol-2-one | 390.48 | 8 |
| 295 | 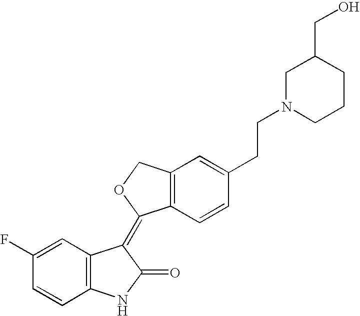 | 5-Fluoro-3-{5-[2-(3- hydroxymethyl- piperidin-1-yl)-ethyl]-3H- isobenzofuran-1- ylidene}-1,3-dihydro- indol-2-one | 408.47 | 8 |
| 296 | 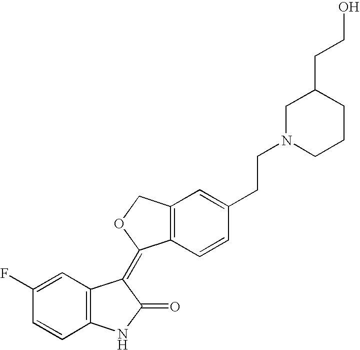 | 5-Fluoro-3-(5-{2-[3-(2- hydroxy-ethyl)-piperidin- 1-yl]-ethyl}-3H- isobenzofuran-1- ylidene)-1,3-dihydro- indol-2-one | 422.497 | 8 |
| 297 | 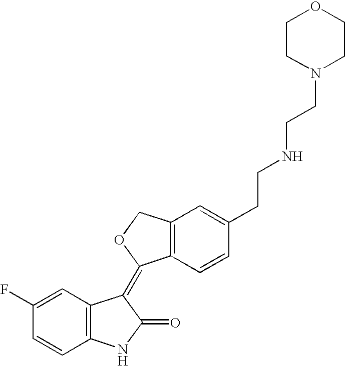 | 5-Fluoro-3-{5-[2-(2- morpholin-4-yl- ethylamino)-ethyl]-3H- isobenzofuran-1- ylidene}-1,3-dihydro- indol-2-one | 423.485 | 8 |
| 298 | 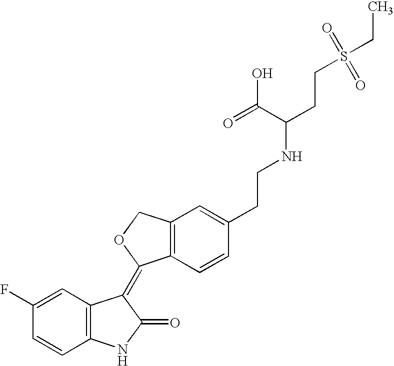 | 4-Ethanesulfonyl-2-{2- [1-(5-fluoro-2-oxo-1,2- dihydro-indol-3-ylidene)- 1,3-dihydro- isobenzofuran-5-yl]- ethylamino}-butyric acid | 488.534 | 8 |
| 299 | 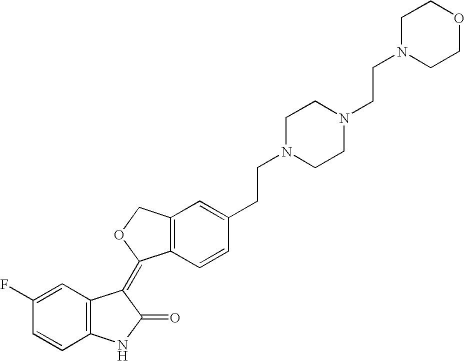 | 5-Fluoro-3-(5-{2-[4-(2- morpholin-4-yl-ethyl)- piperazin-1-yl]-ethyl}- 3H-isobenzofuran-1- ylidene)-1,3-dihydro- indol-2-one | 492.592 | 8 |
| 300 | 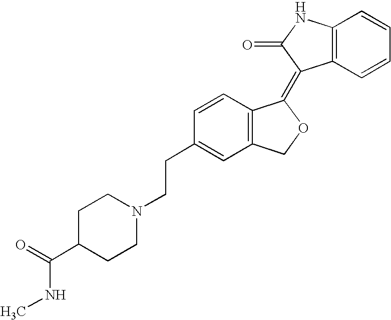 | 1-{2-[1-(2-Oxo-1,2- dihydro-indol-3-ylidene)- 1,3-dihydro- isobenzofuran-5-yl]- ethyl}-piperidine-4- carboxylic acid methylamide | 417.506 | 8 |
| 301 | 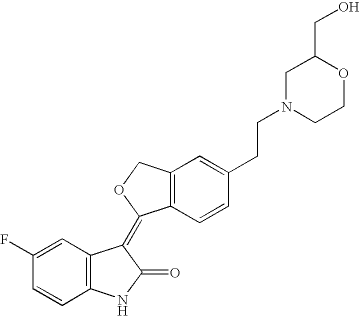 | 5-Fluoro-3-{5-[2-(2- hydroxymethyl- morpholin-4-yl)-ethyl]- 3H-isobenzofuran-1- ylidene}-1,3-dihydro- indol-2-one | 410.443 | 8 |
| 302 | 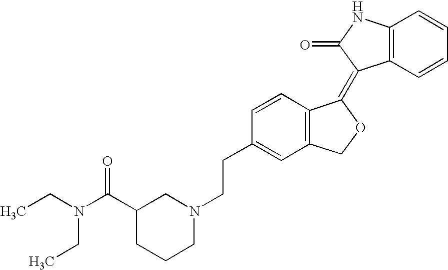 | 1-{2-[1-(2-Oxo-1,2- dihydro-indol-3-ylidene)- 1,3-dihydro- isobenzofuran-5-yl]- ethyl}-piperidine-3- carboxylic acid diethylamide | 459.587 | 8 |
| 303 | 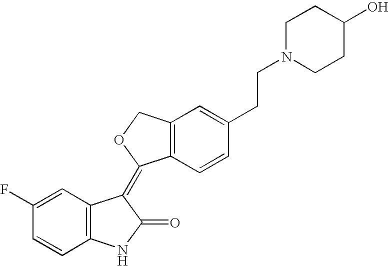 | 5-Fluoro-3-{5-[2-(4- hydroxy-piperidin-1-yl)- ethyl]-3H- isobenzofuran-1- ylidene}-1,3-dihydro- indol-2-one | 394.444 | 8 |
| 304 | 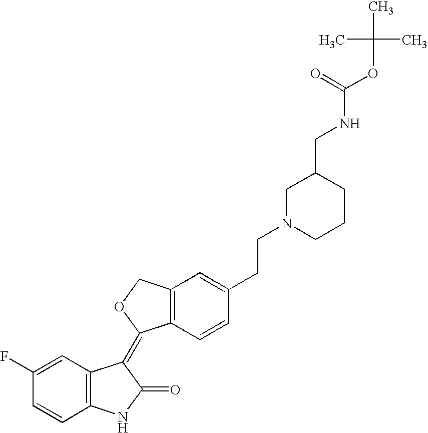 | (1-{2-[1-(5-Fluoro-2- oxo-1,2-dihydro-indol-3- ylidene)-1,3-dihydro- isobenzofuran-5-yl]- ethyl}-piperidin-3- ylmethyl)-carbamic acid tert-butyl ester | 507.603 | 8 |
| 305 | 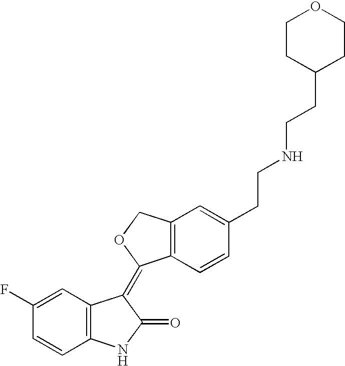 | 5-Fluoro-3-(5-{2-[2- (tetrahydro-pyran-4-yl)- ethylamino]-ethyl}-3H- isobenzofuran-1- ylidene)-1,3-dihydro- indol-2-one | 422.497 | 8 |
| 306 | 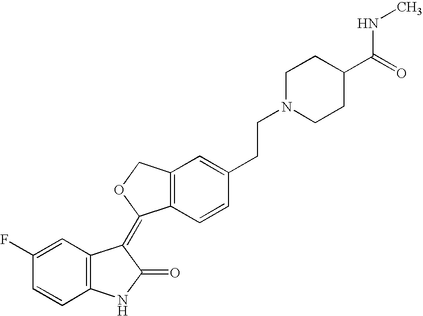 | 1-{2-[1-(5-Fluoro-2-oxo- 1,2-dihydro-indol-3- ylidene)-1,3-dihydro- isobenzofuran-5-yl]- ethyl}-piperidine-4- carboxylic acid methylamide | 435.496 | 8 |
| 307 | 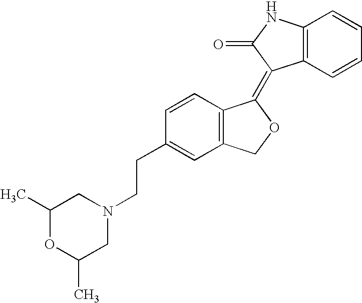 | 3-{5-[2-(2,6-Dimethyl- morpholin-4-yl)-ethyl]- 3H-isobenzofuran-1- ylidene}-1,3-dihydro- indol-2-one | 390.48 | 8 |
| 308 | 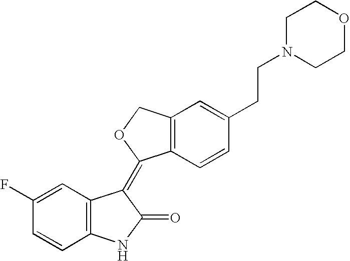 | 5-Fluoro-3-[5-(2- morpholin-4-yl-ethyl)- 3H-isobenzofuran-1- ylidene]-1,3-dihydro- indol-2-one | 380.417 | 8 |
| 309 | 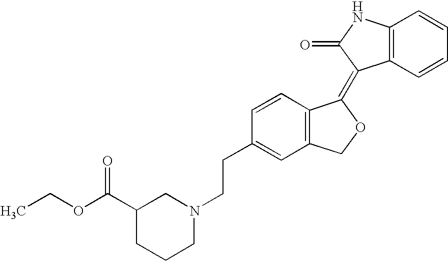 | 1-{2-[1-(2-Oxo-1,2- dihydro-indol-3-ylidene)- 1,3-dihydro- isobenzofuran-5-yl]- ethyl{-piperidine-3- carboxylic acid ethyl ester | 432.517 | 8 |
| 310 | 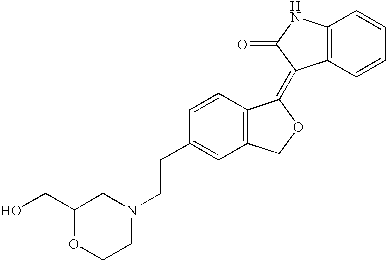 | 3-{5-[2-(2- Hydroxymethyl- morpholin-4-yl)-ethyl]- 3H-isobenzofuran-1- ylidene}-1,3-dihydro- indol-2-one | 392.453 | 8 |
| 311 | 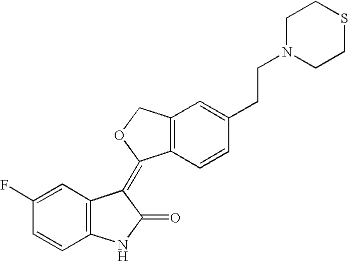 | 5-Fluoro-3-[5-(2- thiomorpholin-4-yl- ethyl)-3H-isobenzofuran-1- ylidene]-1,3-dihydro- indol-2-one | 396.484 | 8 |
| 312 | 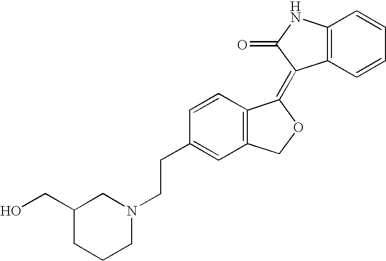 | 3-{5-[2-(3- Hydroxymethyl- piperidin-1-yl)-ethyl]-3H- isobenzofuran-1- ylidene}-1,3-dihydro- indol-2-one | 390.48 | 8 |
| 313 | 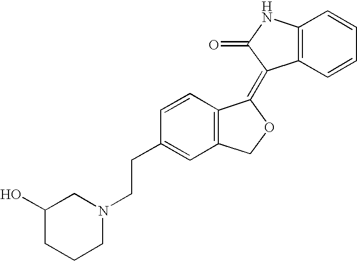 | 3-{5-[2-(3-Hydroxy- piperidin-1-yl)-ethyl]-3H- isobenzofuran-1- ylidene}-1,3-dihydro- indol-2-one | 376.454 | 8 |
| 314 | 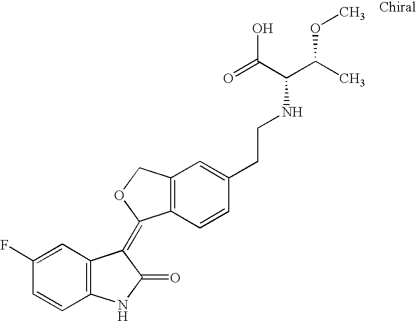 | 2-{(R)-2-[1-(5-Fluoro-2- oxo-1,2-dihydro-indol-3- ylidene)-1,3-dihydro- isobenzofuran-5-yl]- ethylamino}-(R)-3- methoxy-butyric acid | 426.442 | 8 |
| 315 | 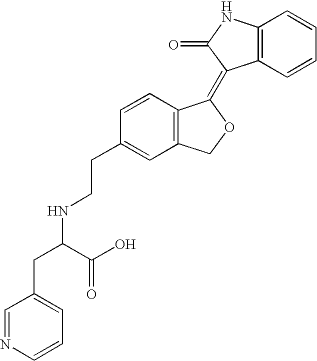 | 2-{2-[1-(2-Oxo-1,2- dihydro-indol-3-ylidene)- 1,3-dihydro- isobenzofuran-5-yl]- ethylamino}-3-pyridin-3- yl-propionic acid | 441.485 | 8 |
| 316 | 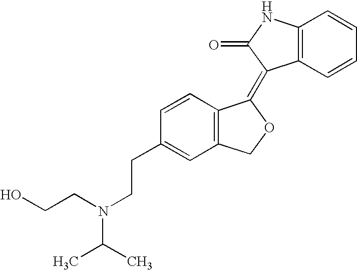 | 3-(5-{2-[(2-Hydroxy- ethyl)-isopropyl-amino]- ethyl}-3H- isobenzofuran-1- ylidene)-1,3-dihydro- indol-2-one | 378.469 | 8 |
| 317 | 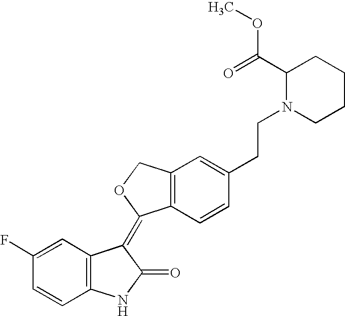 | 1-{2-[1-(5-Fluoro-2-oxo- 1,2-dihydro-indol-3- ylidene)-1,3-dihydro- isobenzofuran-5-yl]- ethyl}-piperidine-2- carboxylic acid methyl ester | 436.48 | 8 |
| 318 | 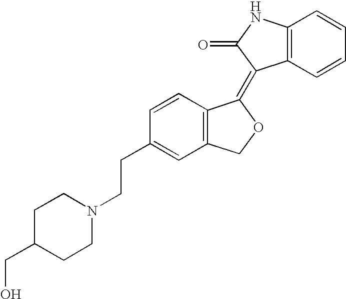 | 3-{5-[2-(4- Hydroxymethyl- piperidin-1-yl)-ethyl]-3H- isobenzofuran-1- ylidene}-1,3-dihydro- indol-2-one | 390.48 | 8 |
| 319 | 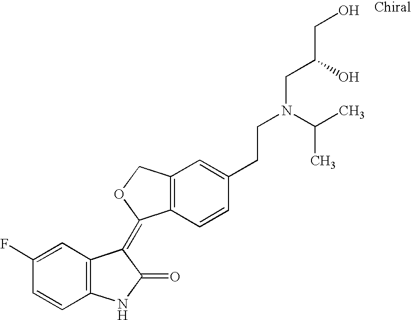 | 3-[5-(2-{[(2S)-2,3- Dihydroxy-propyl]- isopropyl-amino}-ethyl)- 3H-isobenzofuran-1- ylidene]-5-fluoro-1,3- dihydro-indol-2-one | 426.485 | 8 |
| 320 | 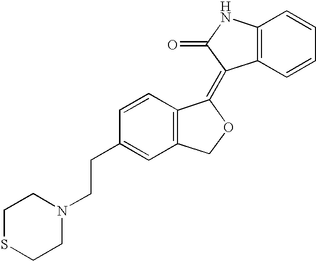 | 3-[5-(2-Thiomorpholin- 4-yl-ethyl)-3H- isobenzofuran-1- ylidene]-1,3-dihydro- indol-2-one | 378.494 | 8 |
| 321 | 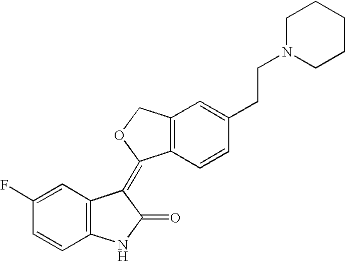 | 5-Fluoro-3-[5-(2- piperidin-1-yl-ethyl)-3H- isobenzofuran-1- ylidene]-1,3-dihydro- indol-2-one | 378.445 | 8 |
| 322 | 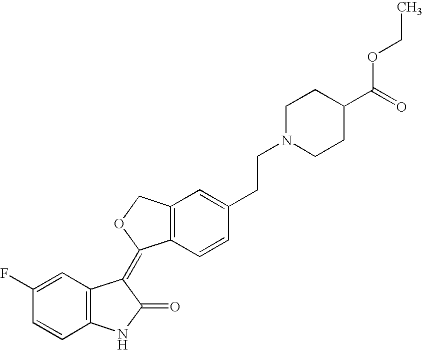 | 1-{2-[1-(5-Fluoro-2-oxo- 1,2-dihydro-indol-3- ylidene)-1,3-dihydro- isobenzofuran-5-yl]- ethyl}-piperidine-4- carboxylic acid ethyl ester | 450.507 | 8 |
| 323 | 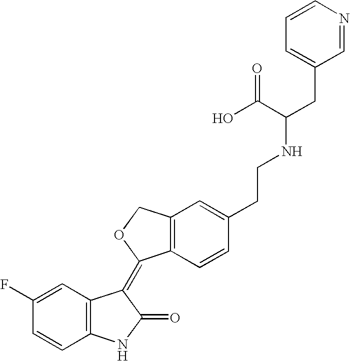 | 2-{2-[1-(5-Fluoro-2-oxo- 1,2-dihydro-indol-3- ylidene)-1,3-dihydro- isobenzofuran-5-yl]- ethylamino}-3-pyridin-3- yl-propionic acid | 459.475 | 8 |
| 324 | 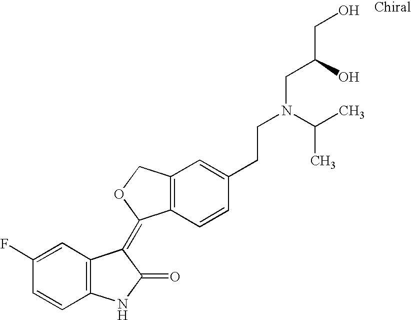 | 3-[5-(2-{[(2R)-2,3- Dihydroxy-propyl]- isopropyl-amino}-ethyl)- 3H-isobenzofuran-1- ylidene]-5-fluoro-1,3- dihydro-indol-2-one | 426.485 | 8 |
| 325 | 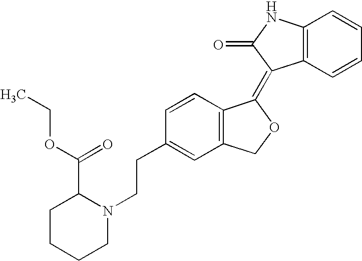 | 1-{2-[1-(2-Oxo-1,2- dihydro-indol-3-ylidene)-1,3- dihydro-isobenzofuran-5-yl]- ethyl}-piperidine-2- carboxylic acid ethyl ester | 432.517 | 8 |
| 326 | 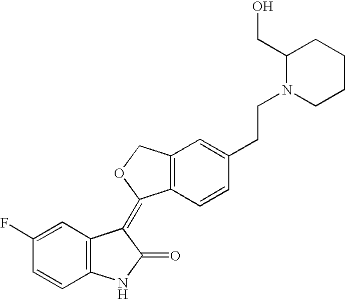 | 5-Fluoro-3-{5-[2-(2- hydroxymethyl- piperidin-1-yl)-ethyl]-3H- isobenzofuran-1- ylidene}-1,3-dihydro- indol-2-one | 408.47 | 8 |
| 327 | 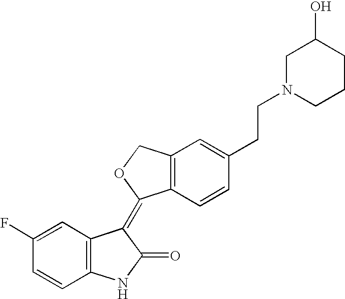 | 5-Fluoro-3-{5-[2-(3- hydroxy-piperidin-1-yl)- ethyl]-3H-isobenzofuran-1- ylidene}-1,3-dihydro- indol-2-one | 394.444 | 8 |
| 328 | 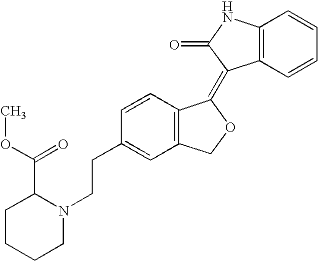 | 1-{2-[1-(2-Oxo-1,2- dihydro-indol-3-ylidene)-1,3- dihydro-isobenzofuran-5-yl]- ethyl}-piperidine-2- carboxylic acid methyl ester | 418.49 | 8 |
| 329 | 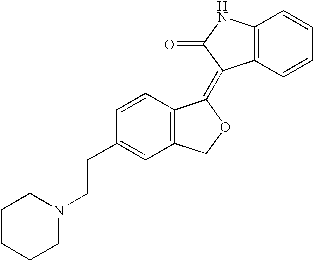 | 3-[5-(2-Piperidin-1-yl- ethyl)-3H-isobenzofuran-1- ylidene]-1,3-dihydro- indol-2-one | 360.455 | 8 |
| 330 | 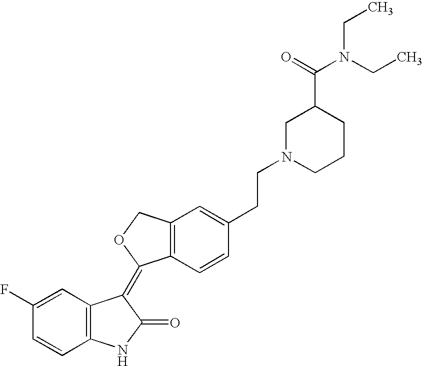 | 1-{2-[1-(5-Fluoro-2-oxo- 1,2-dihydro-indol-3- ylidene)-1,3-dihydro- isobenzofuran-5-yl]- ethyl}-piperidine-3- carboxylic acid diethylamide | 477.577 | 8 |
| 331 | 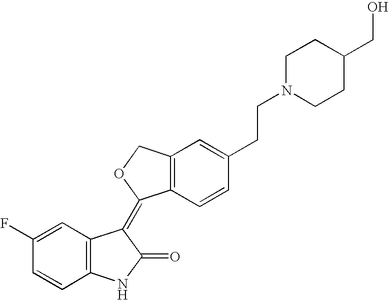 | 5-Fluoro-3-{5-[2-(4- hydroxymethyl- piperidin-1-yl)-ethyl]-3H- isobenzofuran-1- ylidene]-1,3-dihydro- indol-2-one | 408.47 | 8 |
| 332 | 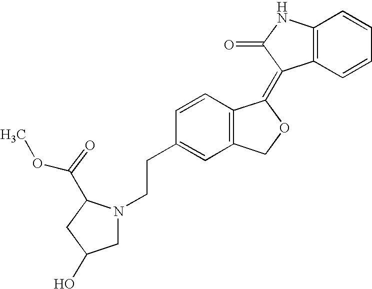 | 4-Hydroxy-1-{2-[1-(2- oxo-1,2-dihydro-indol-3- ylidene)-1,3-dihydro- isobenzofuran-5-yl]- ethyl}-pyrrolidine-2- carboxylic acid methyl ester | 420.463 | 8 |
| 333 | 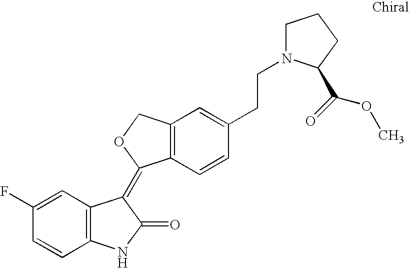 | 1-{2-[1-(5-Fluoro-2-oxo- 1,2-dihydro-indol-3- ylidene)-1,3-dihydro- isobenzofuran-5-yl]- ethyl}-pyrrolidine-(S)-2- carboxylic acid methyl ester | 422.454 | 8 |
| 334 | 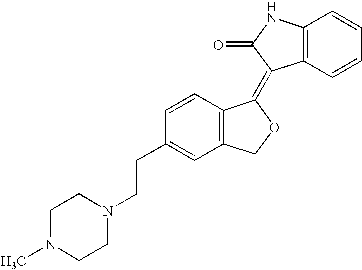 | 3-{5-[2-(4-Methyl- piperazin-1-yl)-ethyl]- 3H-isobenzofuran-1- ylidene}-1,3-dihydro- indol-2-one | 375.47 | 8 |
| 335 | 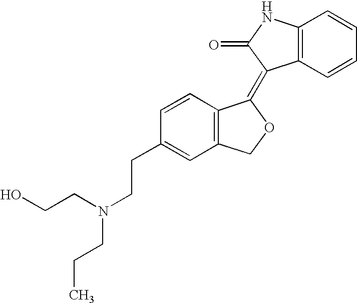 | 3-(5-{2-[(2-Hydroxy- ethyl)-propyl-amino]- ethyl}-3H-isobenzofuran-1- ylidene)-1,3-dihydro- indol-2-one | 378.469 | 8 |
| 336 | 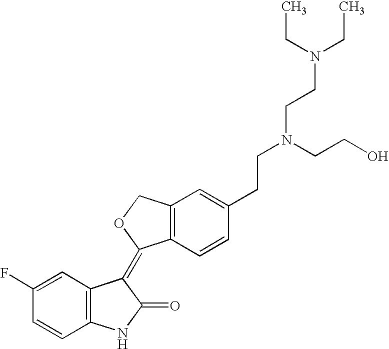 | 3-(5-{2-[(2- Diethylamino-ethyl)-(2- hydroxy-ethyl)-amino]- ethyl)-3H-isobenzofuran-1- ylidene)-5-fluoro-1,3- dihydro-indol-2-one | 453.555 | 8 |
| 337 | 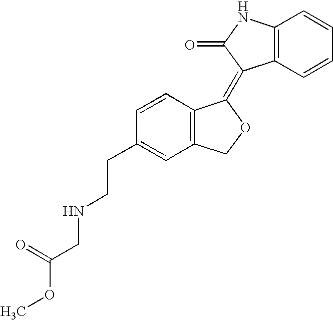 | {2-[1-(2-Oxo-1,2- dihydro-indol-3-ylidene)-1,3- dihydro-isobenzofuran-5-yl]- ethylamino}-acetic acid methyl ester | 364.399 | 8 |
| 338 | 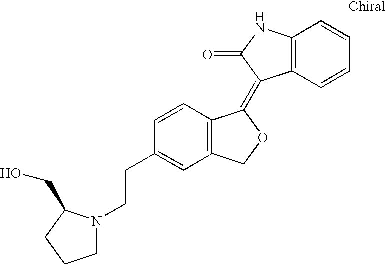 | 3-(5-{2-[(S)-2- Hydroxymethyl- pyrrolidin-1-yl]-ethyl}- 3H-isobenzofuran-1- ylidene)-1,3-dihydro- indol-2-one | 376.454 | 8 |
| 339 | 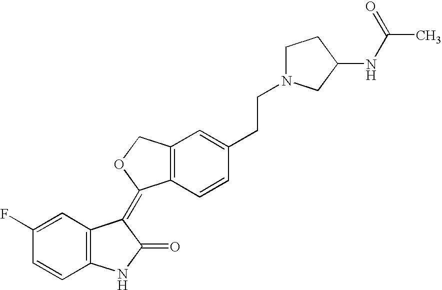 | N-(1-{2-[1-(5-Fluoro-2- oxo-1,2-dihydro-indol-3- ylidene)-1,3-dihydro- isobenzofuran-5-yl]- ethyl}-pyrrolidin-3-yl)- acetamide | 421.47 | 8 |
| 340 | 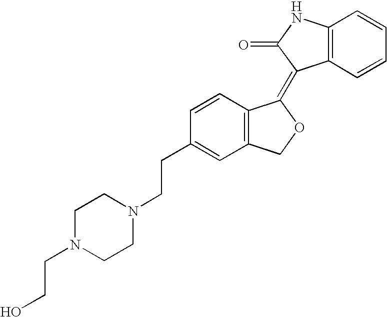 | 3-(5-{2-[4-(2-Hydroxy- ethyl)-piperazin-1-yl]- ethyl}-3H-isobenzofuran-1- ylidene)-1,3-dihydro- indol-2-one | 405.495 | 8 |
| 341 | 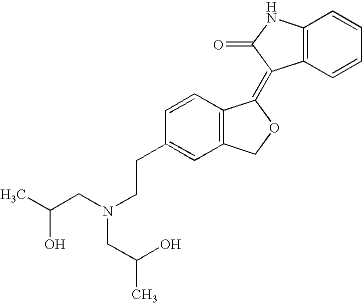 | 3-(5-{2-[Bis-(2-hydroxy- propyl)-amino]-ethyl}- 3H-isobenzofuran-1- ylidene)-1,3-dihydro- indol-2-one | 408.495 | 8 |
| 342 | 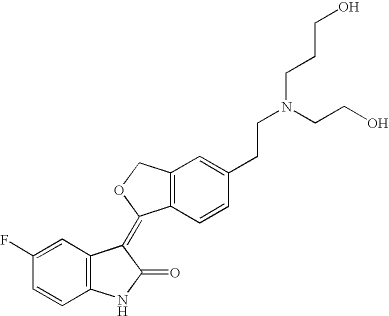 | 5-Fluoro-3-(5-{2-[(2- hydroxy-ethyl)-(3- hydroxy-propyl)-amino]- ethyl}-3H-isobenzofuran-1- ylidene)-1,3-dihydro- indol-2-one | 412.458 | 8 |
| 343 | 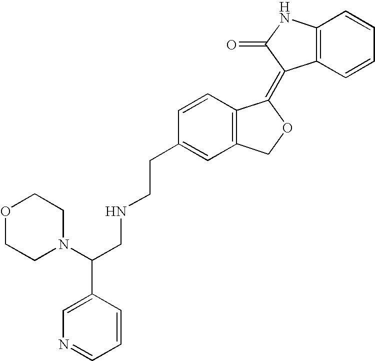 | 3-{5-[2-(2-Morpholin-4- yl-2-pyridin-3-yl- ethylamino)-ethyl]-3H- isobenzofuran-1- ylidene}-1,3-dihydro- indol-2-one | 482.581 | 8 |
| 344 | 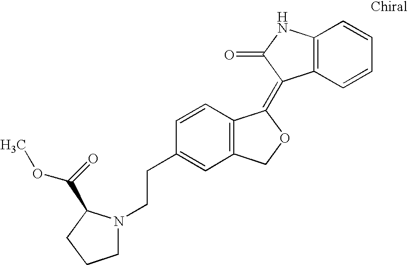 | 1-{2-[1-(2-Oxo-1,2- dihydro-indol-3-ylidene)-1,3- dihydro-isobenzofuran-5-yl]- ethyl}-pyrrolidine-(S)-2- carboxylic acid methyl ester | 404.464 | 8 |
| 345 | 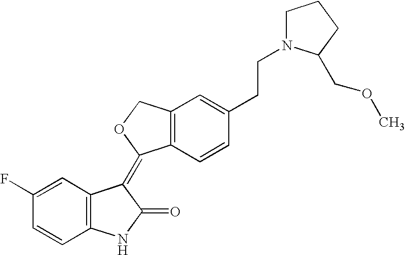 | 5-Fluoro-3-{5-[2-(2- methoxymethyl- pyrrolidin-1-yl)-ethyl]- 3H-isobenzofuran-1- ylidene}-1,3-dihydro- indol-2-one | 408.47 | 8 |
| 346 | 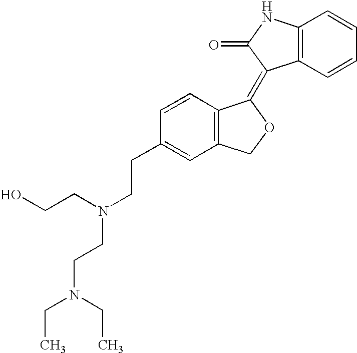 | 3-(5-{2-[(2- Diethylamino-ethyl)-(2- hydroxy-ethyl)-amino]- ethyl}-3H-isobenzofuran-1- ylidene)-1,3-dihydro- indol-2-one | 435.565 | 8 |
| 347 | 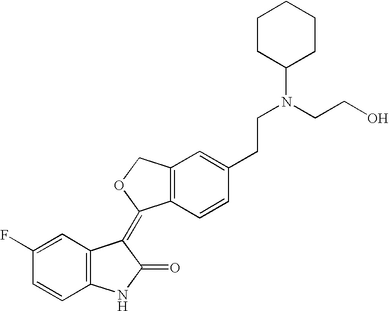 | 3-(5-{2-[Cyclohexyl-(2- hydroxy-ethyl)-amino]- ethyl}-3H-isobenzofuran-1- ylidene)-5-fluoro-1,3- dihydro-indol-2-one | 436.524 | 8 |
| 348 | 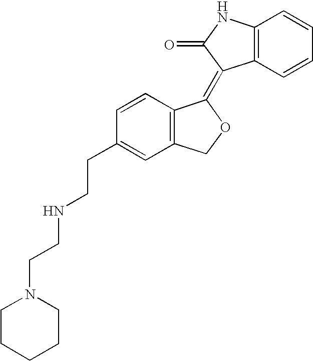 | 3-{5-[2-(2-Piperidin-1-yl- ethylamino)-ethyl]-3H- isobenzofuran-1- ylidene}-1,3-dihydro- indol-2-one | 403.523 | 8 |
| 349 | 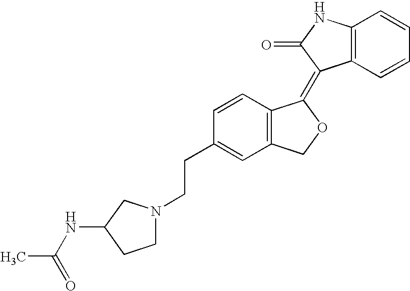 | N-(1-{2-[1-(2-Oxo-1,2- dihydro-indol-3-ylidene)- 1,3-dihydro- isobenzofuran-5-yl]-ethyl}- pyrrolidin-3-yl)-acetamide | 403.479 | 8 |
| 350 | 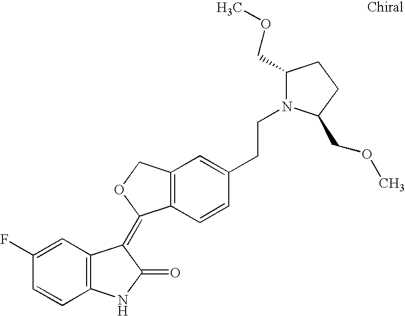 | 3-(5-{2-[(S,S)-2,5-Bis- methoxymethyl- pyrrolidin-1-yl]-ethyl}- 3H-isobenzofuran-1- ylidene)-5-fluoro-1,3- dihydro-indol-2-one | 452.523 | 8 |
| 351 | 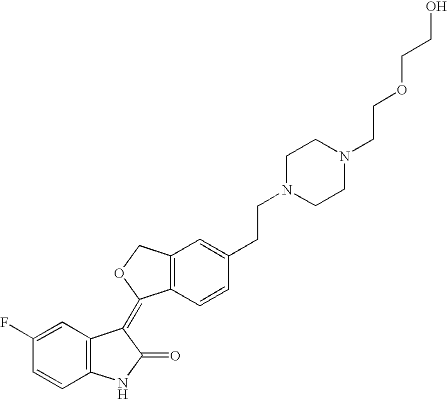 | 5-Fluoro-3-[5-(2-{4-[2- (2-hydroxy-ethoxy)- ethyl]-piperazin-1-yl}- ethyl)-3H-isobenzofuran-1- ylidene]-1,3-dihydro- indol-2-one | 467.538 | 8 |
| 352 | 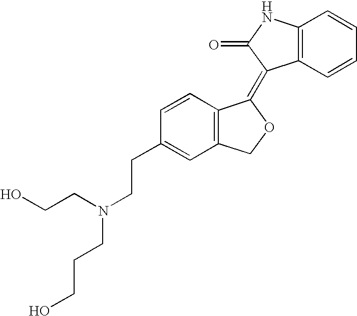 | 3-(5-{2-[(2-Hydroxy- ethyl)-(3-hydroxy- propyl)-amino]-ethyl}- 3H-isobenzofuran-1- ylidene)-1,3-dihydro- indol-2-one | 394.468 | 8 |
| 353 | 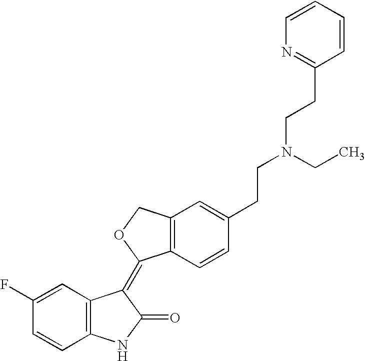 | 3-(5-{2-[Ethyl-(2-pyridin- 2-yl-ethyl)-amino]- ethyl}-3H-isobenzofuran-1- ylidene)-5-fluoro-1,3- dihydro-indol-2-one | 443.519 | 8 |
| 354 | 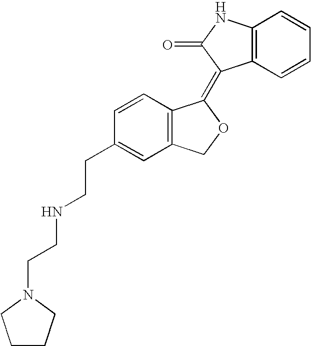 | 3-{5-[2-(2-Pyrrolidin-1- yl-ethylamino)-ethyl]- 3H-isobenzofuran-1- ylidene}-1,3-dihydro- indol-2-one | 389.496 | 8 |
| 355 | 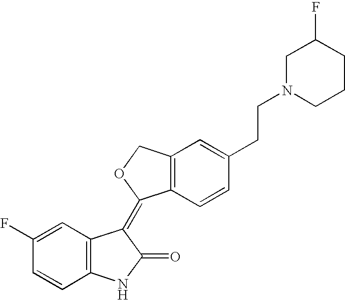 | 5-Fluoro-3-{5-[2-(3- fluoro-piperidin-1-yl)- ethyl]-3H-isobenzofuran-1- ylidene}-1,3-dihydro- indol-2-one | 396.435 | 8 |
| 356 | 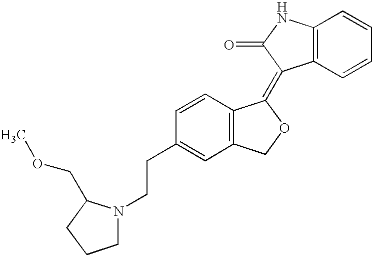 | 3-{5-[2-(2- Methoxymethyl- pyrrolidin-1-yl)-ethyl]- 3H-isobenzofuran-1- ylidene}-1,3-dihydro- indol-2-one | 390.48 | 8 |
| 357 | 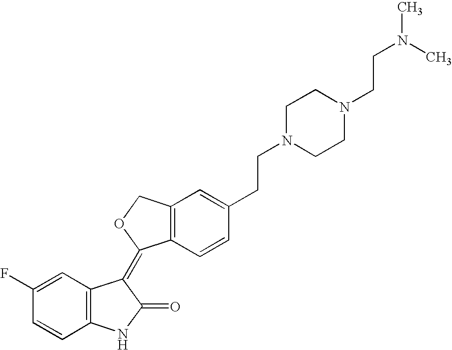 | 3-(5-{2-[4-(2- Dimethylamino-ethyl)- piperazin-1-yl]-ethyl}- 3H-isobenzofuran-1- ylidene)-5-fluoro-1,3- dihydro-indol-2-one | 450.555 | 8 |
| 358 | 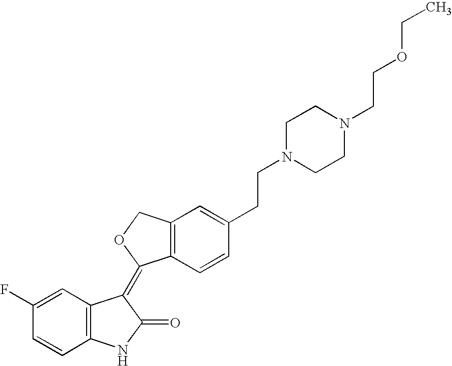 | 3-(5-{2-[4-(2-Ethoxy- ethyl)-piperazin-1-yl]- ethyl}-3H-isobenzofuran-1- ylidene)-5-fluoro-1,3- dihydro-indol-2-one | 451.539 | 8 |
| 359 | 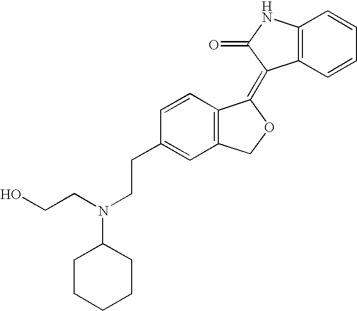 | 3-(5-{2-[Cyclohexyl-(2- hydroxy-ethyl)-amino]- ethyl}-3H-isobenzofuran-1- ylidene)-1,3-dihydro- indol-2-one | 418.534 | 8 |
| 360 | 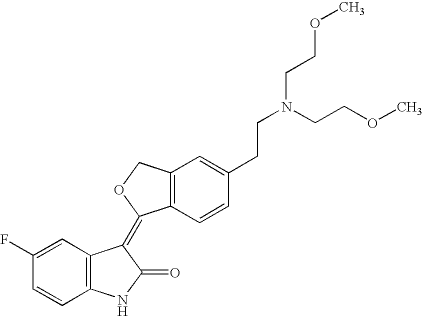 | 3-(5-{2-[Bis-(2-methoxy- ethyl)-amino]-ethyl}-3H- isobenzofuran-1- ylidene)-5-fluoro-1,3- dihydro-indol-2-one | 426.485 | 8 |
| 361 | 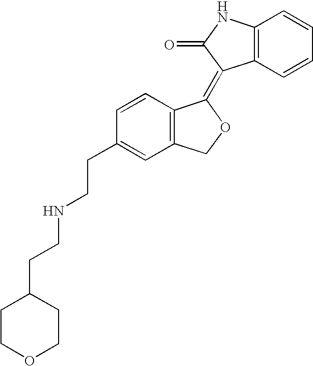 | 3-(5-{2-[2-(Tetrahydro- pyran-4-yl)-ethylamino]- ethyl}-3H-isobenzofuran-1- ylidene)-1,3-dihydro- indol-2-one | 404.507 | 8 |
| 362 | 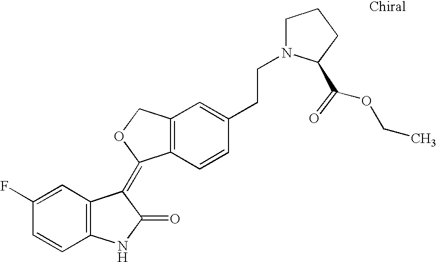 | 1-{2-[1-(5-Fluoro-2-oxo- 1,2-dihydro-indol-3- ylidene)-1,3-dihydro- isobenzofuran-5-yl]- ethyl}-pyrrolidine-(S)-2- carboxylic acid ethyl ester | 436.48 | 8 |
| 363 | 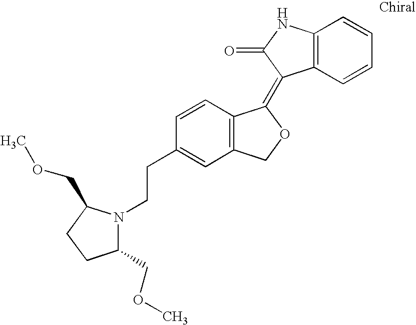 | 3-{5-[2-(S,S)-2,5-Bis- methoxymethyl- pyrrolidin-1-yl)-ethyl]- 3H-isobenzofuran-1- ylidene}-1,3-dihydro- indol-2-one | 434.533 | 8 |
| 364 | 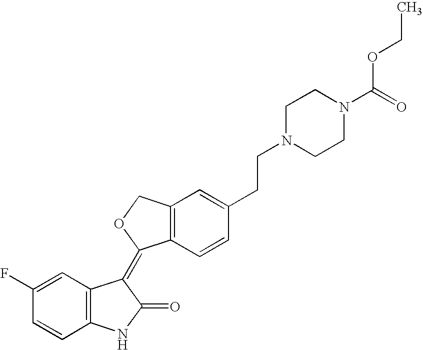 | 4-{2-[1-(5-Fluoro-2-oxo- 1,2-dihydro-indol-3- ylidene)-1,3-dihydro- isobenzofuran-5-yl]- ethyl}-piperazine-1- carboxylic acid ethyl ester | 451.495 | 8 |
| 365 | 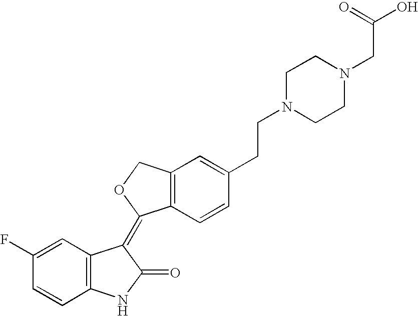 | (4-{2-[1-(5-Fluoro-2- oxo-1,2-dihydro-indol-3- ylidene)-1,3-dihydro- isobenzofuran-5-yl]- ethyl}-piperazin-1-yl)- acetic acid | 437.469 | 8 |
| 366 | 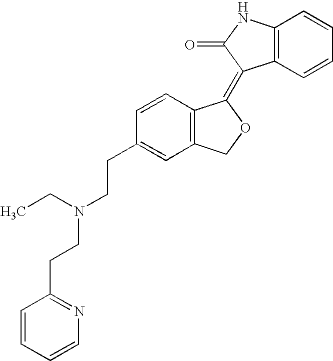 | 3-(5-{2-[Ethyl-(2-pyridin- 2-yl-ethyl)-amino]- ethyl}-3H-isobenzofuran-1- ylidene)-1,3-dihydro- indol-2-one | 425.529 | 8 |
| 367 | 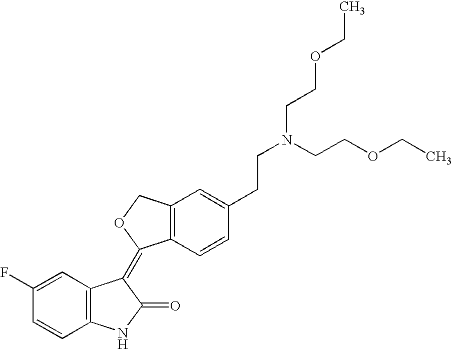 | 3-(5-{2-[Bis-(2-ethoxy- ethyl)-amino]-ethyl}-3H- isobenzofuran-1- ylidene)-5-fluoro-1,3- dihydro-indol-2-one | 454.539 | 8 |
| 368 | 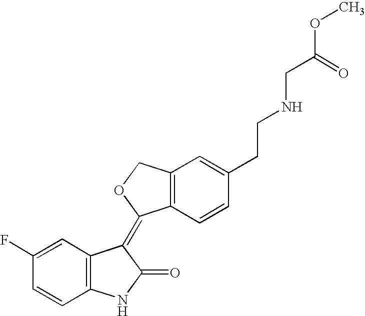 | {2-[1-(5-Fluoro-2-oxo- 1,2-dihydro-indol-3- ylidene)-1,3-dihydro- isobenzofuran-5-yl]- ethylamino}-acetic acid methyl ester | 382.389 | 8 |
| 369 | 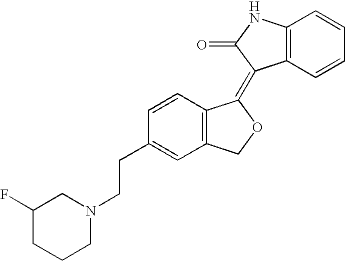 | 3-{5-[2-(3-Fluoro- piperidin-1-yl)-ethyl]-3H- isobenzofuran-1- ylidene}-1,3-dihydro- indol-2-one | 378.445 | 8 |
| 370 | 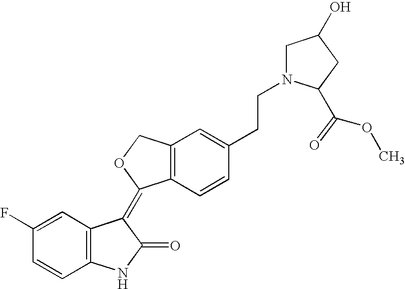 | 1-{2-[1-(5-Fluoro-2-oxo- 1,2-dihydro-indol-3- ylidene)-1,3-dihydro- isobenzofuran-5-yl]- ethyl}-4-hydroxy- pyrrolidine-2-carboxylic acid ester | 438.453 | 8 |
| 371 | 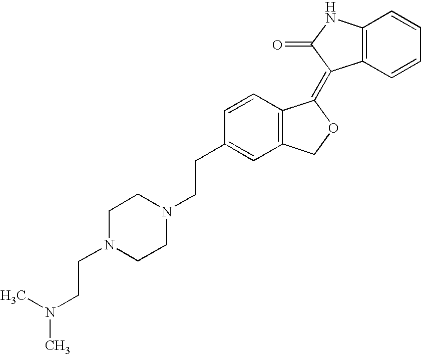 | 3-(5-{2-[4-(2- Dimethylamino-ethyl)- piperazin-1-yl]-ethyl}- 3H-isobenzofuran-1- ylidene)-1,3-dihydro- indol-2-one | 432.565 | 8 |
| 372 | 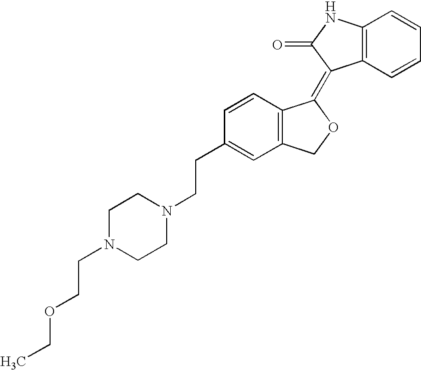 | 3-(5-{2-[4-(2-Ethoxy- ethyl)-piperazin-1-yl]- ethyl)-3H-isobenzofuran- 1-ylidene)-1,3-dihydro- indol-2-one | 433.549 | 8 |
| 373 | 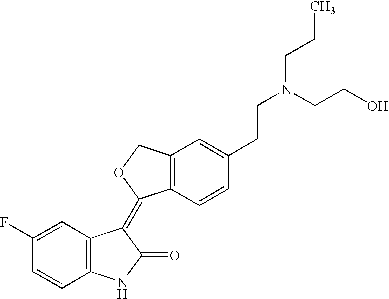 | 5-Fluoro-3-(5-{2-[(2- hydroxy-ethyl)-propyl- amino]-ethyl}-3H- isobenzofuran-1- ylidene)-1,3-dihydro- indol-2-one | 396.46 | 8 |
| 374 | 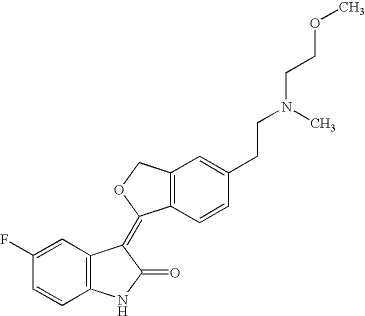 | 5-Fluoro-3-(5-{2-[(2- methoxy-ethyl)-methyl- amino]-ethyl}-3H- isobenzofuran-1- ylidene)-1,3-dihydro- indol-2-one | 382.433 | 8 |
| 375 | 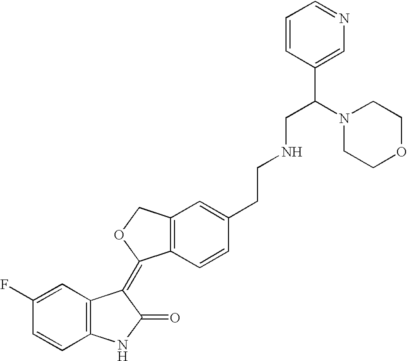 | 5-Fluoro-3-{5-[2-(2- morpholin-4-yl-2- pyridin-3-yl-ethylamino)- ethyl]-3H-isobenzofuran-1- ylidene}-1,3-dihydro- indol-2-one | 500.571 | 8 |
| 376 | 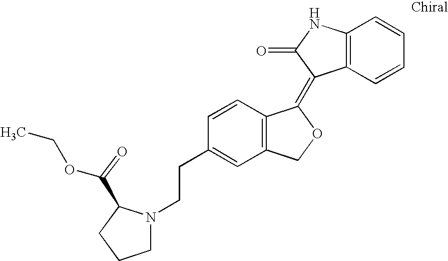 | 1-{2-[1-(2-Oxo-1,2- dihydro-indol-3-ylidene)- 1,3-dihydro-isobenzofuran-5- yl]-ethyl}-pyrrolidine-(R)-2- carboxylic acid ethyl ester | 418.49 | 8 |
| 377 | 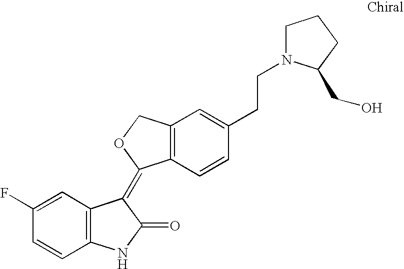 | 5-Fluoro-3-(5-{2-[(S)-2- hydroxymethyl- pyrrolidin-1-yl]-ethyl}- 3H-isobenzofuran-1- ylidene)-1,3-dihydro- indol-2-one | 394.444 | 8 |
| 378 | 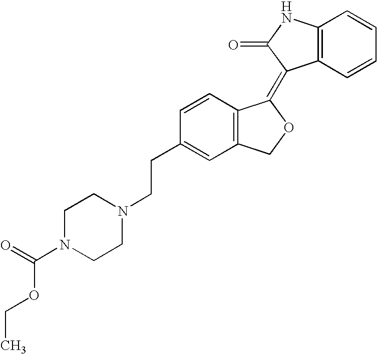 | 4-{2-[1-(2-Oxo-1,2- dihydro-indol-3-ylidene)- 1,3-dihydro- isobenzofuran-5-yl]- ethyl}-piperazine-1- carboxylic acid ethyl ester | 433.505 | 8 |
| 379 | 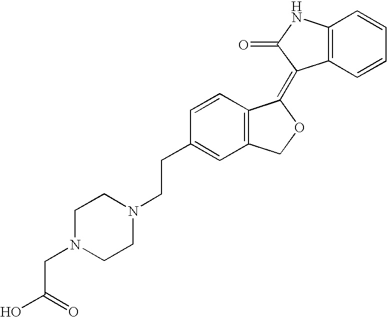 | (4-{2-[1-(2-Oxo-1,2- dihydro-indol-3-ylidene)- 1,3-dihydro- isobenzofuran-5-yl]- ethyl}-piperazin-1-yl)- acetic acid | 419.478 | 8 |
| 380 | 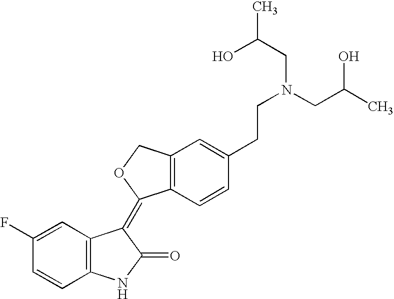 | 3-(5-{2-[Bis-(2-hydroxy- propyl)-amino]-ethyl}- 3H-isobenzofuran-1- ylidene)-5-fluoro-1,3- dihydro-indol-2-one | 426.485 | 8 |
| 381 | 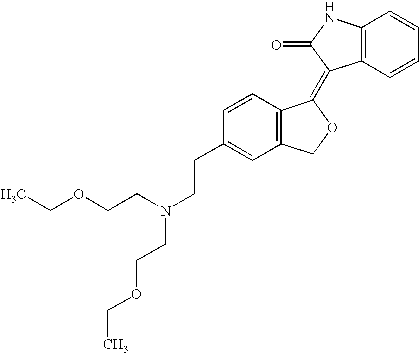 | 3-(5-{2-[Bis-(2-ethoxy- ethyl)-amino]-ethyl}-3H- isobenzofuran-1- ylidene)-1,3-dihydro- indol-2-one | 436.549 | 8 |
| 382 | 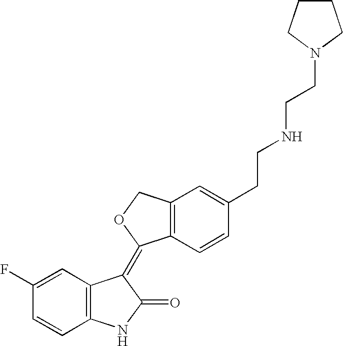 | 5-Fluoro-3-{5-[2-(2- pyrrolidin-1-yl- ethylamino)-ethyl]-3H- isobenzofuran-1- ylidene}-1,3-dihydro- indol-2-one | 407.486 | 8 |
| 383 | 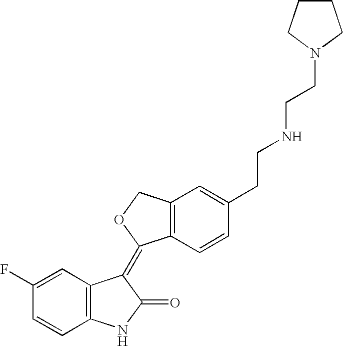 | 3-(5-{2-[(Tetrahydro- pyran-4-ylmethyl)- amino]-ethyl}-3H- isobenzofuran-1- ylidene)-1,3-dihydro- indol-2-one | 390.48 | 8 |
| 384 | 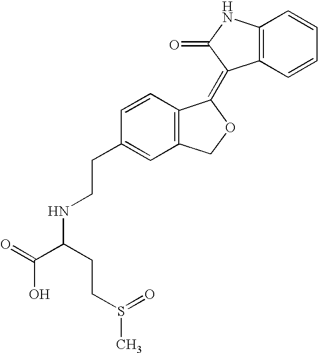 | 4-Methanesulfinyl-2-{2- [1-(2-oxo-1,2-dihydro- indol-3-ylidene)-1,3- dihydro-isobenzofuran- 5-yl]-ethylamino}- butyric acid | 440.518 | 8 |
| 385 | 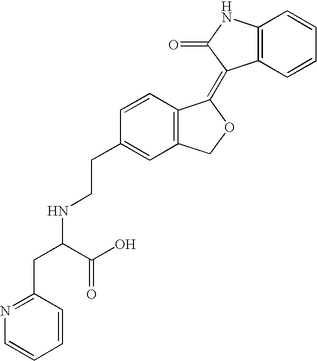 | 2-{2-[1-(2-Oxo-1,2- dihydro-indol-3-ylidene)- 1,3-dihydro- isobenzofuran-5-yl]- ethylamino}-3-pyridin-2- yl-propionic acid | 441.485 | 8 |
| 386 | 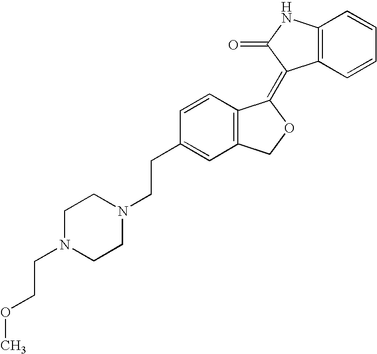 | 3-(5-{2-[4-(2-Methoxy- ethyl)-piperazin-1-yl]- ethyl}-3H-isobenzofuran-1- ylidene)-1,3-dihydro- indol-2-one | 419.522 | 8 |
| 387 | 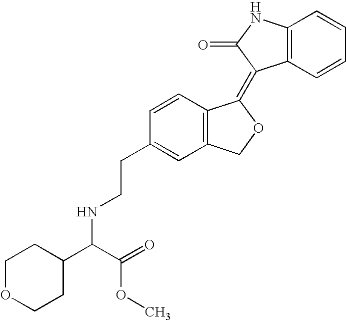 | {2-[1-(2-Oxo-1,2- dihydro-indol-3-ylidene)- 1,3-dihydro- isobenzofuran-5-yl]- ethylamino}-(tetrahydro- pyran-4-yl)-acetic acid methyl ester | 448.516 | 8 |
| 388 | 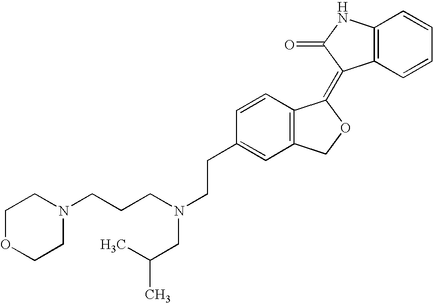 | 3-(5-{2-[Isobutyl-(3- morpholin-4-yl-propyl)- amino]-ethyl}-3H- isobenzofuran-1- ylidene)-1,3-dihydro- indol-2-one | 475.629 | 8 |
| 389 | 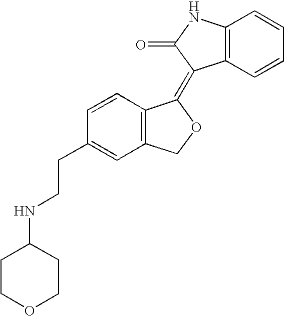 | 3-{5-[2-(Tetrahydro- pyran-4-ylamino)-ethyl]- 3H-isobenzofuran-1- ylidene}-1,3-dihydro- indol-2-one | 376.454 | 8 |
| 390 | 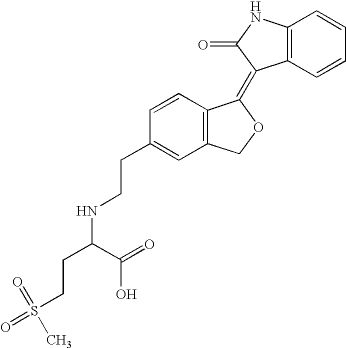 | 4-Methanesulfonyl-2-{2- [1-(2-oxo-1,2-dihydro- indol-3-ylidene)-1,3- dihydro-isobenzofuran- 5-yl]-ethylamino}- butyric acid | 456.517 | 8 |
| 391 | 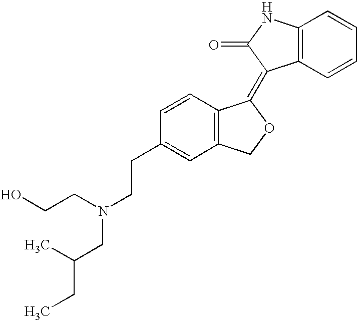 | 3-(5-{2-[(2-Hydroxy- ethyl)-(2-methyl-butyl)- amino]-ethyl}-3H- isobenzofuran-1- ylidene)-1,3-dihydro- indol-2-one | 406.523 | 8 |
| 392 | 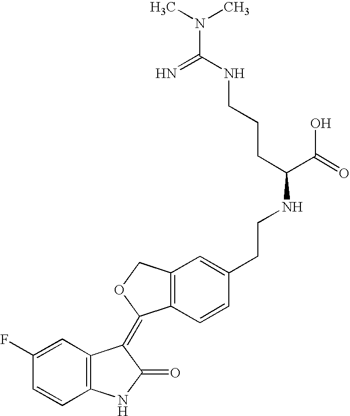 | 5-(N′,N′-Dimethyl- guanidino)-(S)-2-{2-[1- (5-fluoro-2-oxo-1,2- dihydro-indol-3-ylidene)-1,3- dihydro-isobenzofuran-5-yl- ethylamino}-pentanoic acid | 495.552 | 8 |
| 393 | 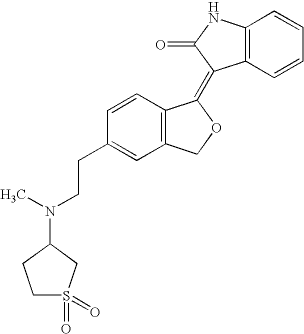 | 3-(5-{2-[(1,1-Dioxo- tetrahydro-1lambda*6*- thiophen-3-yl)-methyl- amino]-ethyl}-3H- isobenzofuran-1- ylidene)-1,3-dihydro- indol-2-one | 424.519 | 8 |
| 394 | 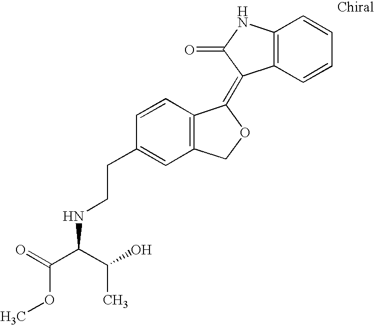 | (S)-3-Hydroxy-(S)-2-{2- [1-(2-oxo-1,2-dihydro- indol-3-ylidene)-1- dihydro-isobenzofuran- 5-yl]-ethylamino}- butyric acid methyl ester | 408.452 | 8 |
| 395 | 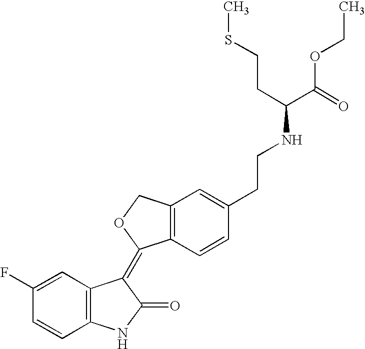 | 2-{(S)-2-[1-(5-Fluoro-2- oxo-1,2-dihydro-indol-3- ylidene)-1,3-dihydro- isobenzofuran-5-yl]- ethylamino}-4- methylsulfanyl-butyric acid ethyl ester | 470.562 | 8 |
| 396 | 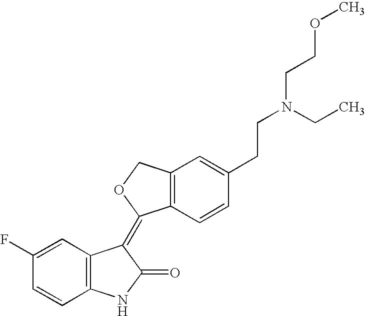 | 3-(5-{2-[Ethyl-(2- methoxy-ethyl)-amino]- ethyl}-3H-isobenzofuran-1- ylidene)-5-fluoro-1,3- dihydro-indol-2-one | 396.46 | 8 |
| 397 | 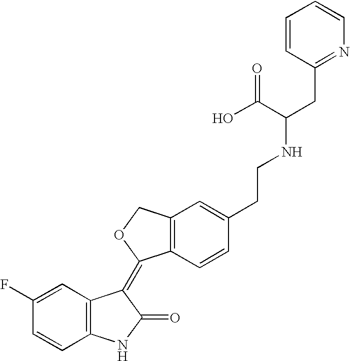 | 2-{2-[1-(5-Fluoro-2-oxo- 1,2-dihydro-indol-3- ylidene)-1,3-dihydro- isobenzofuran-5-yl]- ethylamino}-3-pyridin-2- yl-propionic acid | 459.475 | 8 |
| 398 | 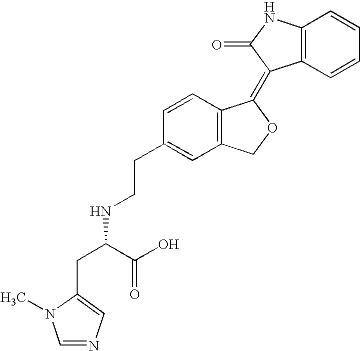 | 3-(3-Methyl-3H- imidazol-4-yl)-(S)-2-{2- [1-(2-oxo-1,2-dihydro- indol-3-ylidene)-1,3- dihydro-isobenzofuran- 5-yl]-ethylamino}- propionic acid | 444.489 | 8 |
| 399 | 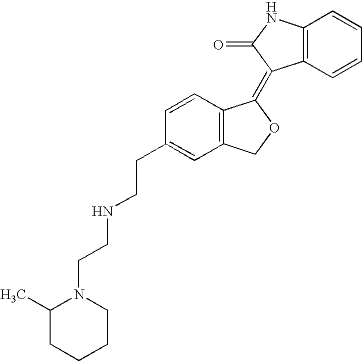 | 3-(5-{2-[2-(2-Methyl- piperidin-1-yl)- ethylamino]-ethyl}-3H- isobenzofuran-1- ylidene)-1,3-dihydro- indol-2-one | 417.55 | 8 |
| 400 | 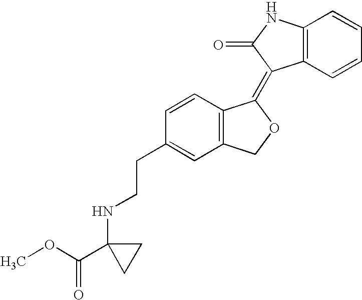 | 1-{2-[1-(2-Oxo-1,2- dihydro-indol-3-ylidene)- 1,3-dihydro- isobenzofuran-5-yl]- ethylamino}- cyclopropanecarboxylic acid methyl ester | 390.437 | 8 |
| 401 | 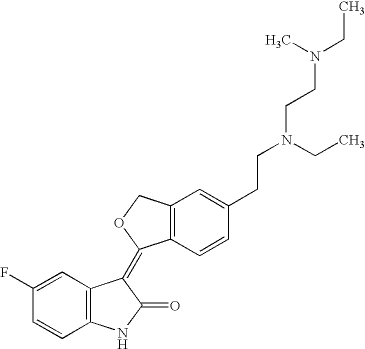 | 3-[5-(2-{Ethyl-[2-(ethyl- methyl-amino)-ethyl]- amino}-ethyl)-3H- isobenzofuran-1- ylidene]-5-fluoro-1,3- dihydro-indol-2-one | 423.529 | 8 |
| 402 | 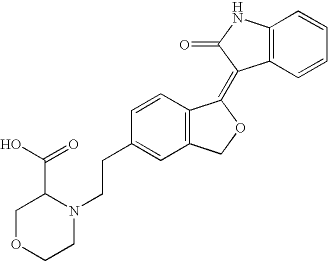 | 4-{2-[1-(2-Oxo-1,2- dihydro-indol-3-ylidene)- 1,3-dihydro- isobenzofuran-5-yl]- ethyl}-morpholine-3- carboxylic acid | 406.436 | 8 |
| 403 | 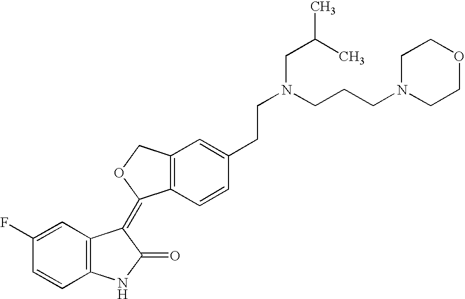 | 5-Fluoro-3-(5-{2- [isobutyl-(3-morpholin- 4-yl-propyl)-amino]- ethyl}-3H-isobenzofuran-1- ylidene)-1,3-dihydro- indol-2-one | 493.619 | 8 |
| 404 | 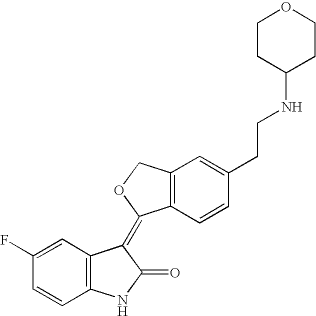 | 5-Fluoro-3-{5-[2- (tetrahydro-pyran-4- ylamino)-ethyl]-3H- isobenzofuran-1- ylidene}-1,3-dihydro- indol-2-one | 394.444 | 8 |
| 405 | 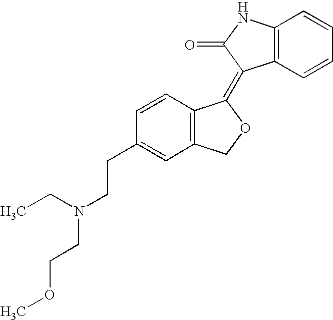 | 3-(5-{2-[Ethyl-(2- methoxy-ethyl)-amino]- ethyl}-3H- isobenzofuran-1- ylidene)-1,3-dihydro- indol-2-one | 378.469 | 8 |
| 406 | 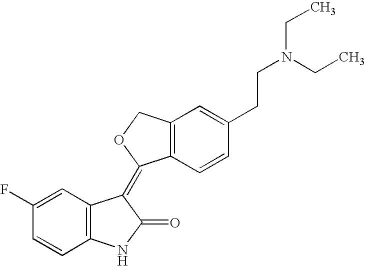 | 3-[5-(2-Diethylamino- ethyl)-3H-isobenzofuran-1- ylidene]-5-fluoro-1,3- dihydro-indol-2-one | 366.434 | 8 |
| 407 | 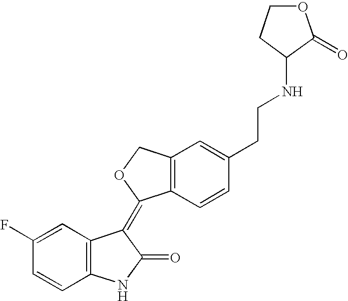 | 5-Fluoro-3-{5-[2-(2-oxo- tetrahydro-furan-3- ylamino)-ethyl]-3H- isobenzofuran-1- ylidene}-1,3-dihydro- indol-2-one | 394.4 | 8 |
| 408 | 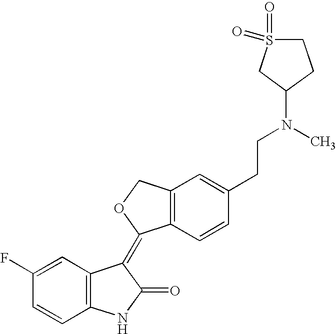 | 3-(5-{2-[(1,1-Dioxo- tetrahydro-1lambda*6*- thiophen-3-yl)-methyl- amino]-ethyl}-3H- isobenzofuran-1- ylidene)-5-fluoro-1,3- dihydro-indol-2-one | 442.509 | 8 |
| 409 | 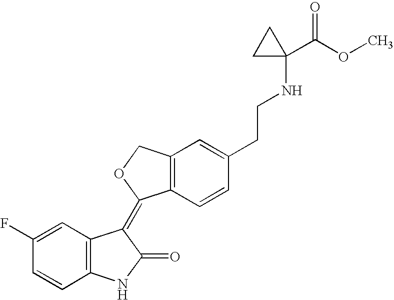 | 1-{2-[1-(5-Fluoro-2-oxo- 1,2-dihydro-indol-3- ylidene)-1,3-dihydro- isobenzofuran-5-yl]- ethylamino}- cyclopropanecarboxylic acid methyl ester | 408.427 | 8 |
| 410 | 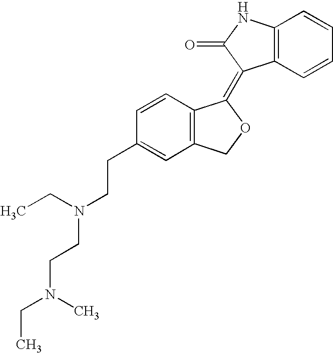 | 3-[5-(2-{Ethyl-[2-(ethyl- methyl-amino)-ethyl]- amino}-ethyl)-3H- isobenzofuran-1- ylidene]-1,3-dihydro- indol-2-one | 405.539 | 8 |
| 411 | 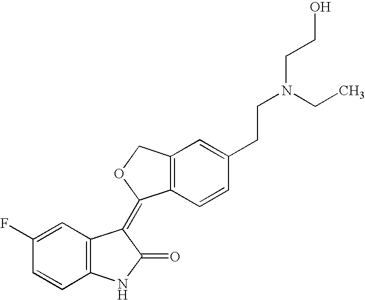 | 3-(5-{2-[Ethyl-(2- hydroxy-ethyl)-amino]- ethyl}-3H-isobenzofuran-1- ylidene)-5-fluoro-1,3- dihydro-indol-2-one | 382.433 | 8 |
| 412 | 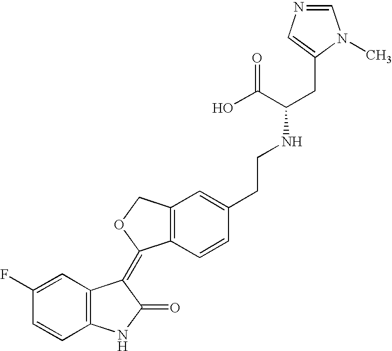 | (S)-2-{2-[1-(5-Fluoro-2- oxo-1,2-dihydro-indol-3- ylidene)-1,3-dihydro- isobenzofuran-5-yl]- ethylamino}-3-(3- methyl-3H-imidazol-4- yl)-propionic acid | 462.479 | 8 |
| 413 | 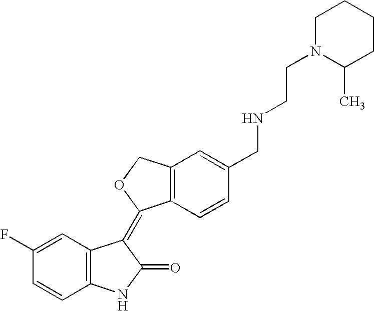 | 5-Fluoro-3-(5-{[2-(2- methyl-piperidin-1-yl)- ethylamino]-methyl}-3H- isobenzofuran-1- ylidene)-1,3-dihydro- indol-2-one | 421.513 | 8 |
| 414 | 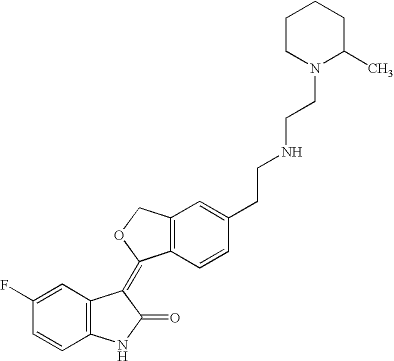 | 5-Fluoro-3-(5-{2-[2-(2- methyl-piperidin-1-yl)- ethylamino]-ethyl}-3H- isobenzofuran-1- ylidene)-1,3-dihydro- indol-2-one | 435.54 | 8 |
| 415 | 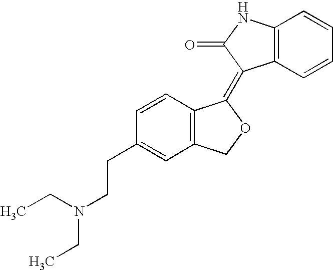 | 3-[5-(2-Diethylamino- ethyl)-3H-isobenzofuran-1- ylidene]-1,3-dihydro- indol-2-one | 348.444 | 8 |
| 416 | 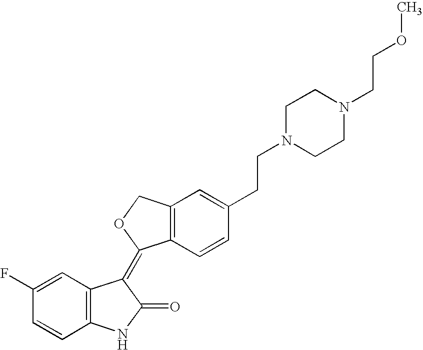 | 5-Fluoro-3-(5-{2-[4-(2- methoxy-ethyl)- piperazin-1-yl]-ethyl}- 3H-isobenzofuran-1- ylidene)-1,3-dihydro- indol-2-one | 437.512 | 8 |
| 417 | 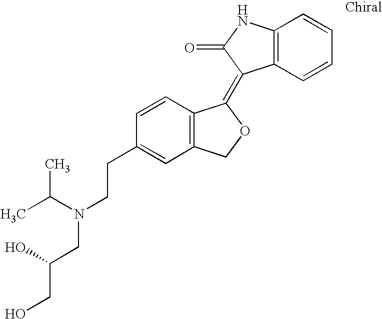 | 3-[5-(2-{[(2S)-2,3- Dihydroxy-propyl]- isopropyl-amino}-ethyl)- 3H-isobenzofuran-1- ylidene]-1,3-dihydro- indol-2-one | 408.495 | 8 |
| 418 | 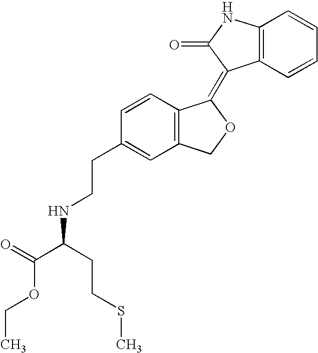 | 4-Methylsulfanyl-(S)-2- {2-[1-(2-oxo-1,2- dihydro-indol-3-ylidene)-1,3- dihydro-isobenzofuran-5-yl]- ethylamino}-butyric acid ethyl ester | 452.572 | 8 |
| 419 | 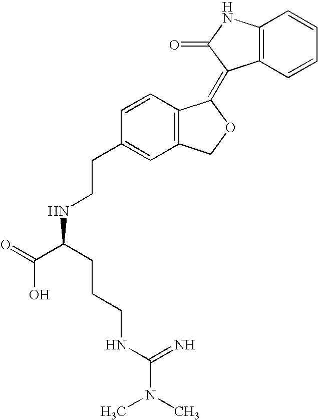 | 5-(N′,N′-Dimethyl- guanidino)-(S)-2-{2-[1- (2-oxo-1,2-dihydro- indol-3-ylidene)-1,3- dihydro-isobenzofuran- 5-yl]-ethylamino}- pentanoic acid | 477.562 | 8 |
| 420 | 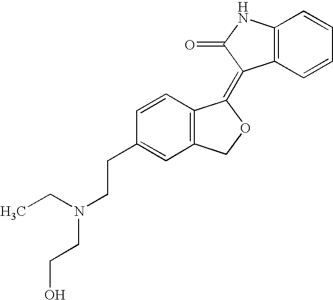 | 3-(5-{2-[Ethyl-(2- hydroxy-ethyl)-amino]- ethyl}-3H-isobenzofuran-1- ylidene)-1,3-dihydro- indol-2-one | 364.443 | 8 |
| 421 | 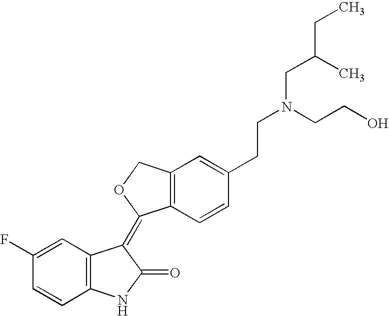 | 5-Fluoro-3-(5-{2-[(2- hydroxy-ethyl)-(2- methyl-butyl)-amino]- ethyl}-3H-isobenzofuran-1- ylidene)-1,3-dihydro- indol-2-one | 424.513 | 8 |
| 422 | 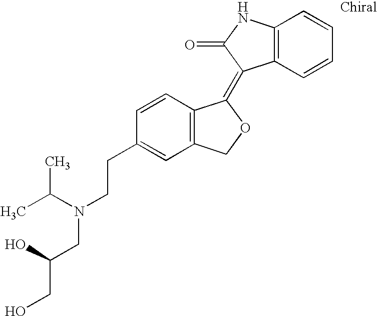 | 3-[5-(2-{[(2R)-2,3- Dihydroxy-propyl]- isopropyl-amino}-ethyl)- 3H-isobenzofuran-1- ylidene]-1,3-dihydro- indol-2-one | 408.495 | 8 |

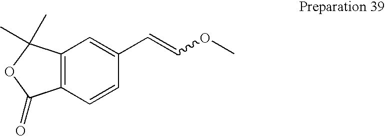

### 5-(2-methoxy-vinyl)-3,3-dimethyl-3H-isobenzofuran-1-one

Experimental procedure similar to Preparation 36.

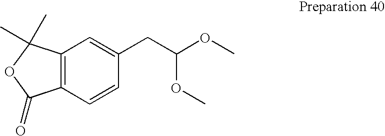

### Preparation of 5-(2,2-dimethoxy-ethyl)-3,3-dimethyl-3H-isobenzofuran-1-one

To a stirred solution of 5-(2-methoxy-vinyl)-3,3-dimethyl-3H-isobenzofuran-1-one (390 mg, 1.79 mmol) in 7 ml of anhydrous methanol was added 3 drops of concentrated sulfuric acid. The mixture was heated at reflux for 5 hours, cooled and diluted with ethyl acetate (100 ml). The EtOAc solution was washed with saturated NaHCO₃ solution (2×75 ml), dried over Na₂SO₄. Purification of the concentrated filtrate through silica gel column by elution with 10-20% ethyl acetate/hexanes gave the title compound as a clear oil (117 mg, 26%).

¹H NMR (500 MHz, CDCl₃) δ ppm 1.66 (s, 6H) 3.04 (d, J=5.37 Hz, 2H) 3.37 (s, 6H) 4.57 (t, J=5.37 Hz, 1H) 7.26 (s, 1H) 7.39 (d, J=7.81 Hz, 1H) 7.79 (d, J=7.81 Hz, 1H)

### Example 423

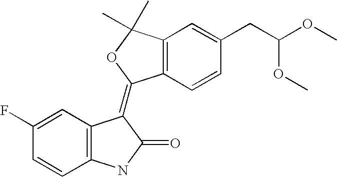

### 3-[5-(2,2-Dimethoxy-ethyl)-3,3-dimethyl-3H-isobenzofuran-1-ylidene]-5-fluoro-1,3-dihydro-indol-2-one

To a stirred solution of 5-fluorooxindole (142 mg, 0.94 mmol) in anhydrous THF (15 ml) under nitrogen was added 1M LiHMDS/THF solution (1.9 ml, 1.9 mmol). The mixture was stirred at room temperature for 10 minutes, and 5-(2,2-dimethoxy-ethyl)-3,3-dimethyl-3H-isobenzofuran-1-one (117 mg, 0.47 mmol) was added. The reaction was stirred at room temperature for 2 hours and quenched with 1M sulfuric acid (8 ml). The mixture was poured into 100 ml of water and stirred for 16 hours. The precipitates were filtered, washed with water, and dried under vacuum to the title compound (170 mg, 94%) as a yellow solid.

¹H NMR (500 MHz, DMSO-d6) δ ppm 1.72 (s, 6H) 3.01 (d, J=5.86 Hz, 2H) 3.27 (s, 6H) 4.67 (t, J=5.86 Hz, 1H) 6.79 (dd, J=8.54, 4.64 Hz, 1H) 6.91-6.96 (m, 1H) 7.46 (dd, J=8.30, 1.46 Hz, 1H) 7.56 (s, 1H) 7.59 (dd, J=9.52, 2.69 Hz, 1H) 9.48 (d, J=8.30 Hz, 1H) 10.41 (s, 1H)

### Example 424

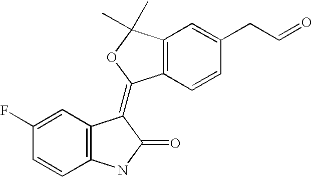

### [1-(5-Fluoro-2-oxo-1,2-dihydro-indol-3-ylidene)-3,3-dimethyl-1,3-dihydro-isobenzofuran-5-yl]-acetaldehyde

To a stirred solution of 3-[5-(2,2-dimethoxy-ethyl)-3,3-dimethyl-3H-isobenzofuran-1-ylidene]-5-fluoro-1,3-dihydro-indol-2-one (160 mg, 0.42 mmol) in 10 ml of THF, was added 0.5 ml of sulfuric acid. The mixture was heated at 60° C. for 30 minutes, cooled to room temp., and poured into 100 ml of water with stirring. After continuously stirred for 16 hours, the mixture was filtered, and washed with water. Removal of the solvent gave the title compound as a yellow solid (140 mg, 99%).

### Example 425

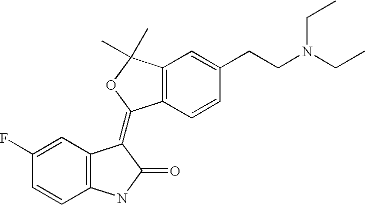

### 3-[5-(2-Diethylamino-ethyl)-3,3-dimethyl-3H-isobenzofuran-1-ylidene]-5-fluoro-1,3-dihydro-indol-2-one

A mixture of [1-(5-fluoro-2-oxo-1,2-dihydro-indol-3-ylidene)-3,3-dimethyl-1,3-dihydro-isobenzofuran-5-yl]-acetaldehyde (60 mg, 0.18 mmol), diethylamine (30 mg, 0.41 mmol), acetic acid (2 drops) and sodium cyanoborohydride (25 mg, 0.40 mmol) in 5 ml of methanol was stirred at room temperature for 16 hours. The mixture was poured into 120 ml of water, and adjusted to approximately pH=9 with saturated NaHCO₃ solution. The resulting precipitates were filtered, washed with water and dried. The resulting crude product was purified through silica gel chromatography with a gradient of MeOH in CHCl₃ to the title compound as a yellow solid (17 mg).

¹H NMR (500 MHz, DMSO-d6) δ ppm 0.96 (t, J=7.08 Hz, 6H) 1.72 (s, 6H) 2.53 (q, J=7.08 Hz, 4H) 2.68-2.73 (m, 2H) 2.80-2.85 (m, 2H) 6.79 (dd, J=8.30, 4.88 Hz, 1H) 6.90-6.95 (m, 1H) 7.43 (dd, J=8.30, 1.46 Hz, 1H) 7.55 (s, 1H) 7.59 (dd, J=9.52, 2.68 Hz, 1H) 9.47 (d, J=7.81 Hz, 1H) 10.40 (s, 1H)

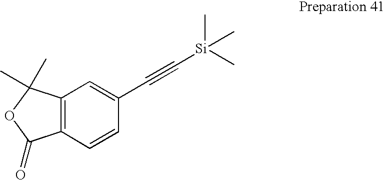

### Preparation of 3,3-dimethyl-5-trimethylsilanylethynyl-3H-isobenzofuran-1-one

A mixture of trifluoromethanesulfonic acid 3,3-dimethyl-1-oxo-1,3-dihydro-isobenzofuran-5-yl ester (8.8 g, 28.4 mmol), trimethylsilylacetylene (10 ml, 72.2 mmol), tetrakis(triphenylphosphine)palladium (656 mg, 0.57 mmol), copper(I) iodide (325 mg, 1.70 mmol) and triethylamine (7.9 ml, 56.8 mmol) in DMF (40 ml) was stirred under nitrogen for 30 minutes. The mixture was diluted with Et₂O and filtered through celite. The filtrate solution was washed with brine (3×100 ml), and concentrated. Purification of the resulting mixture through silica gel chromatography, by elution with a gradient of ethyl acetate in hexanes gave the title compound as a white solid (7.2 g, 98%).

¹H NMR (500 MHz, CDCl₃) δ ppm −0.00 (s, 9H) 1.37 (s, 6H) 7.20 (s, 1H) 7.29 (d, J=7.81 Hz, 1H) 7.51 (d, J=7.81 Hz, 1H)

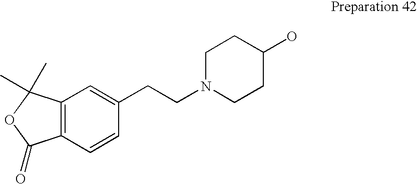

### Preparation of 5-[2-(4-hydroxy-piperidin-1-yl)-ethyl]-3,3-dimethyl-3H-isobenzofuran-1-one

To a cooled solution of 3,3-dimethyl-5-trimethylsilanylethynyl-3H-isobenzofuran-1-one (7.2 g, 27.9 mmol) in a 1:1 MeOH/CH₂Cl₂ mixture (30 ml/30 ml) at 0° C. was added potassium carbonate (5.0 g, 36.2 mmol). The mixture was stirred for 15 minutes, filtered through celite and evaporated to dryness to give a crude product of 5-ethynyl-3,3-dimethyl-3H-isobenzofuran-1-one intermediate as brown solid (5 g), which was directly used for the next step without further purification. A mixture of this intermediate (5 g), sodium cyanoborohydride (3.4 g, 54 mmol) and 4-hydroxypiperidine (8.2 g, 81 mmol) in MeOH (60 ml) was purged with nitrogen, sealed and heated at 120° C. for 16 hours. The mixture was cooled to room temperature, concentrated and purified by silica gel chromatography, eluted with a gradient of MeOH in CHCl₃, to give the title compound as light brown solid (8.41 g).

¹H NMR (500 MHz, CDCl₃) δ ppm 1.52-1.64 (m, 2H) 1.65 (s, 6H) 1.89-1.97 (m, 2H) 2.25 (t, J=9.52 Hz, 2H) 2.60-2.65 (m, 2H) 2.82-2.88 (m, 2H) 2.89-2.94 (m, 2H) 3.74 (br. s., 1H) 7.21 (s, 1H) 7.34 (d, J=7.81 Hz, 1H) 7.76 (d, J=7.81 Hz, 1H)

### Example 426

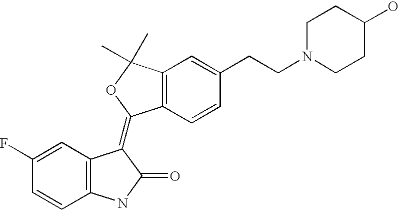

### 5-Fluoro-3-{5-[2-(4-hydroxy-piperidin-1-yl)-ethyl]-3,3-dimethyl-3H-isobenzofuran-1-ylidene}-1,3-dihydro-indol-2-one

To a stirred solution of 5-fluorooxindole (7.86 g, 52 mmol) in anhydrous THF (60 ml) under nitrogen was added 1.0M LiHMDS/THF solution (104 ml, 104 mmol). The mixture was stirred at room temperature for 15 minutes, and then 5-[2-(4-hydroxy-piperidin-1-yl)-ethyl]-3,3-dimethyl-3H-isobenzofuran-1-one (5.0 g, 17.3 mmol) was added. After stirring at room temperature for 2 hours, the mixture was quenched with 50 ml of 2.5M H₂SO₄, heated at 65° C. for 30 minutes and poured into 500 ml of water. The mixture was basified with 5M NaOH to about pH=9 and continuously stirred at room temperature for 16 hours. The resulting solids were filtered, rinsed with water, dried under vacuum to give the title compound as a yellow solid (4.74 g, 65%).

¹H NMR (500 MHz, DMSO-d6) δ ppm 1.34-1.43 (m, 2H) 1.69-1.76 (m, 8H) 2.10 (br. s., 2H) 2.57 (t, J=7.32 Hz, 2H) 2.76-2.83 (m, 2H) 2.87 (t, J=7.32 Hz, 2H) 3.40-3.49 (m, 1H) 4.55 (d, J=4.39 Hz, 1H) 6.79 (dd, J=8.30, 4.39 Hz, 1H) 6.93 (td, J=9.03, 2.93 Hz, 1H) 7.43 (d, J=8.30 Hz, 1H) 7.53 (s, 1H) 7.59 (dd, J=9.28, 2.44 Hz, 1H) 9.47 (d, J=8.30 Hz, 1H) 10.40 (s, 1H)

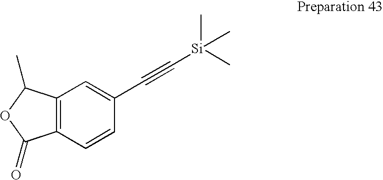

### Preparation of 3-methyl-5-trimethylsilanylethynyl-3H-isobenzofuran-1-one

Experimental procedure similar to Preparation 41.

1H NMR (500 MHz, CDCl₃) δ ppm 0.00 (s, 9H) 1.35 (d, J=6.83 Hz, 3H) 5.25 (q, J=6.83 Hz, 1H) 7.24 (s, 1H) 7.31 (d, J=7.81 Hz, 1H) 7.54 (d, J=7.81 Hz, 1H)

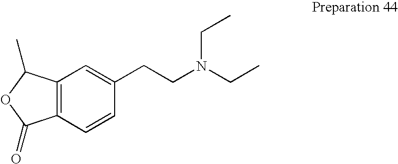

### Preparation of 5-(2-diethylamino-ethyl)-3-methyl-3H-isobenzofuran-1-one

Experimental procedure similar to Preparation 42.

1H NMR (500 MHz, CDCl₃) δ ppm 1.08 (t, J=6.83 Hz, 6H) 1.65 (d, J=6.35 Hz, 3H) 2.64 (br. s., 4H) 2.76 (br. s., 2H) 2.90 (br. s., 2H) 5.54 (q, J=6.83 Hz, 1H) 7.27 (s, 1H) 7.37 (d, J=7.81 Hz, 1H) 7.82 (d, J=7.81 Hz, 1H)

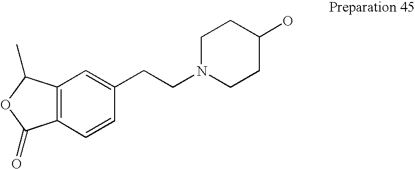

### Preparation of 5-[2-(4-hydroxy-piperidin-1-yl)-ethyl]-3-methyl-3H-isobenzofuran-1-one

Experimental procedure similar to Preparation 42.

1H NMR (500 MHz, CDCl₃) δ ppm 1.50-1.63 (m, 2H) 1.66 (d, J=6.35 Hz, 3H) 1.73 (br. s., 2H) 2.04 (br. s., 2H) 2.79 (br. s., 2H) 2.99 (br. s., 2H) 3.04 (br. s., 2H) 3.89 (br. s., 1H) 5.55 (q, J=6.67 Hz, 1H) 7.33 (s, 1H) 7.38 (d, J=7.81 Hz, 1H) 7.83 (d, J=7.81 Hz, 1H)

The following Examples 427-430 were prepared using the experiment procedure described in Example 426, but with the appropriate reagent, reaction conditions and reactant substitutions that will be readily realized by those of ordinary skill in this art without the exercise of undue experimentation.

### Example 427

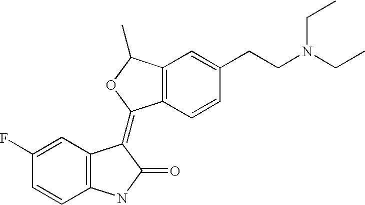

### 3-[5-(2-Diethylamino-ethyl)-3-methyl-3H-isobenzofuran-1-ylidene]-5-fluoro-1,3-dihydro-indol-2-one

¹H NMR (500 MHz, DMSO-d6) δ ppm 0.96 (t, J=7.08 Hz, 6H) 1.67 (d, J=6.83 Hz, 3H) 2.52 (q, J=7.08 Hz, 4H) 2.66-2.71 (m, 2H) 2.82 (t, J=7.57 Hz, 2H) 6.04 (q, J=6.35 Hz, 1H) 6.79 (dd, J=8.30, 4.39 Hz, 1H) 6.90-6.95 (m, 1H) 7.44 (d, J=8.30 Hz, 1H) 7.51 (s, 1H) 7.58 (dd, J=9.76, 2.93 Hz, 1H) 9.49 (d, J=8.30 Hz, 1H) 10.40 (s, 1H)

### Example 428

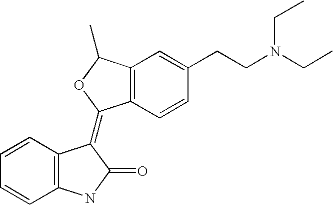

### 3-[5-(2-Diethylamino-ethyl)-3-methyl-3H-isobenzofuran-1-ylidene]-1,3-dihydro-indol-2-one

¹H NMR (500 MHz, DMSO-d6) δ ppm 0.96 (t, J=7.08 Hz, 6H) 1.66 (d, J=6.35 Hz, 3H) 2.53 (q, J=7.08 Hz, 4H) 2.66-2.73 (m, 2H) 2.82 (t, J=7.57 Hz, 2H) 6.01 (q, J=6.67 Hz, 1H) 6.82 (d, J=7.32 Hz, 1H) 6.95 (td, J=7.57, 0.98 Hz, 1H) 7.10 (td, J=7.57, 0.98 Hz, 1H) 7.43 (d, J=8.79 Hz, 1H) 7.49 (s, 1H) 7.84 (d, J=7.81 Hz, 1H) 9.50 (d, J=8.30 Hz, 1H) 10.38 (s, 1H)

### Example 429

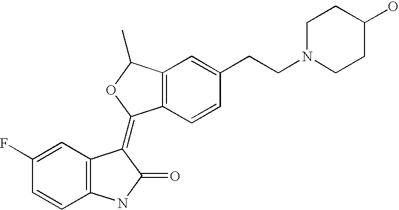

### 5-Fluoro-3-{5-[2-(4-hydroxy-piperidin-1-yl)-ethyl]-3-methyl-3H-isobenzofuran-1-ylidene}-1,3-dihydro-indol-2-one

¹H NMR (500 MHz, DMSO-d6) δ ppm 1.38 (q, J=9.60 Hz, 2H) 1.64-1.75 (m, 5H) 2.09 (br. s., 2H) 2.56 (t, J=7.57 Hz, 2H) 2.79 (br. s., 2H) 2.87 (t, J=7.32 Hz, 2H) 3.40-3.49 (m, J=8.54, 4.64 Hz, 1H) 4.54 (d, J=3.91 Hz, 1H) 6.04 (q, J=6.51 Hz, 1H) 6.79 (dd, J=8.54, 4.64 Hz, 1H) 6.93 (td, J=9.03, 2.93 Hz, 1H) 7.45 (d, J=8.30 Hz, 1H) 7.51 (s, 1H) 7.59 (dd, J=9.76, 2.44 Hz, 1H) 9.50 (d, J=8.30 Hz, 1H) 10.41 (s, 1H)

### Example 430

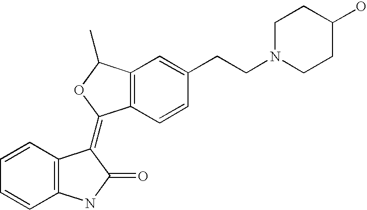

### 3-{5-[2-(4-Hydroxy-piperidin-1-yl)-ethyl]-3-methyl-3H-isobenzofuran-1-ylidene}-1,3-dihydro-indol-2-one

¹H NMR (500 MHz, DMSO-d6) δ ppm 1.32-1.43 (m, 2H) 1.66 (d, J=6.83 Hz, 3H) 1.68-1.75 (m, 2H) 2.09 (br. s., 2H) 2.55 (t, J=7.32 Hz, 2H) 2.78 (br. s., 2H) 2.86 (t, J=7.32 Hz, 2H) 3.40-3.47 (m, 1H) 4.54 (d, J=3.91 Hz, 1H) 6.01 (q, J=6.83 Hz, 1H) 6.82 (d, J=7.81 Hz, 1H) 6.96 (td, J=7.57, 0.98 Hz, 1H) 7.10 (td, J=7.69, 1.22 Hz, 1H) 7.43 (d, J=8.30 Hz, 1H) 7.49 (s, 1H) 7.84 (d, J=7.32 Hz, 1H) 9.50 (d, J=8.30 Hz, 1H) 10.38 (s, 1H)

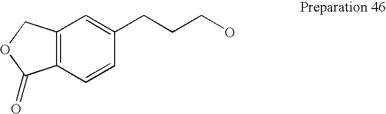

### Preparation of 5-(3-hydroxy-propyl)-3H-isobenzofuran-1-one

A mixture of 5-[3-(tetrahydro-pyran-2-yloxy)-propyl]-3H-isobenzofuran-1-one (1.86 g) and Dowex 50WX8-200 (2.0 g) in 50 ml of methanol was stirred in a 50° C.-bath for 1 hour. The mixture was filtered, washed with methanol, and then evaporated to give the title compound as a solid (1.27 g).

¹H NMR (500 MHz, CDCl₃) δ ppm 1.92-1.99 (m, 2H) 2.86-2.91 (t, J=7.81 Hz, 2H) 3.72 (t, J=6.35 Hz, 2H) 5.30 (s, 2H) 7.34 (s, 1H) 7.39 (d, J=7.81 Hz, 1H) 7.86 (d, J=7.81 Hz, 1H)

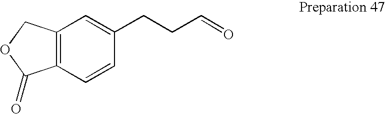

### Preparation of 3-(1-oxo-1,3-dihydro-isobenzofuran-5-yl)-propionaldehyde

To anhydrous CH₂Cl₂ (30 ml) in a −78° C.-bath was added 2M oxalyl chloride/CH₂Cl₂ solution (6.35 ml, 12.7 mmol). Anhydrous DMSO (1.80 ml, 25.4 mmol) was slowly added via syringe. After the mixture was stirred for 5 minutes, a solution of 5-(3-hydroxy-propyl)-3H-isobenzofuran-1-one (1.22 g, 6.35 mmol) in anhydrous CH₂Cl₂ (5 ml) was added. The mixture was stirred at −78° C. for 15 minutes, followed by addition of triethylamine (5.3 ml, 38 mmol). After stirring at −78° C. for another 5 minutes, the mixture was allowed to be warmed up to room temperature and water (50 ml) was added. The aqueous layer was extracted with CH₂Cl₂ (50 ml) and the organic layers were combined, washed with brine (2×75 ml) and dried over Na₂SO₄. Filtration and removal of the solvent gave the title compound as an off-white solid (1.20 g, 99%).

¹H NMR (500 MHz, CDCl₃) δ ppm 2.89 (t, J=7.32 Hz, 2H) 3.11 (t, J=7.32 Hz, 2H) 5.30 (s, 2H) 7.35 (s, 1H) 7.39 (d, J=7.32 Hz, 1H) 7.86 (d, J=8.30 Hz, 1H) 9.85 (s, 1H)

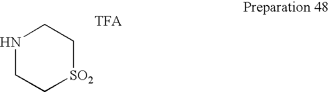

### Preparation of thiomorpholine 1,1-dioxide trifluoroacetic acid salt

To a 0° C. stirred solution of thiomorpholine-4-carboxylic acid tert-butyl ester (3.8 g, 18.7 mmol) in CH₂Cl₂ (100 ml), was added mCPBA (70% purity, 13.8 g, 56.0 mmol). The mixture was first stirred at 0° C. for 20 minutes, then at room temperature for 2 hours, and finally cooled to 0° C. again. Calcium hydroxide (fine powder form, 14.0 g, 189 mmol) was added. After stirring at 0° C. for 10 minutes and then at room temperature for 2 hours, the mixture was filtered through celite, and then washed with CH₂Cl₂. Evaporation of the filtrate provided 1,1-dioxidothiomorpholine-4-carboxylic acid tert-butyl ester as a white solid (4.9 g, >100%).

To a stirred solution of 1,1-dioxidothiomorpholine-4-carboxylic acid tert-butyl ester (4.9 g, 20.8 mmol) in CH₂Cl₂ (25 ml) at 0° C. was added trifluoroacetic acid (15 ml). After stirred at room temperature for 1 hour, the mixture was evaporated to give an oil. Diethyl ether (100 ml) was added, and the white precipitate was separated. The upper solution was then removed by decanting. This procedure was repeated three times to ensure complete removal of excess trifluoroacetic acid. The remaining white solids were dried in vacuo to give the title compound (5.12 g, 99%).

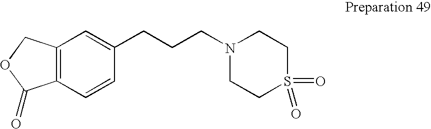

### 5-[3-(1,1-dioxidothiomorpholin-4-yl)-propyl]-3H-isobenzofuran-1-one

A mixture of 3-(1-oxo-1,3-dihydro-isobenzofuran-5-yl)-propionaldehyde (1.20 g, 6.31 mmol), thiomorpholine 1,1-dioxide trifluoroacetic acid salt (1.79 g, 7.2 mmol), and sodium cyanoborohydride (452 mg, 7.2 mmol) in 50 ml of methanol was stirred at room temperature for 30 minutes. The reaction mixture was concentrated and purified by silica gel chromatography (eluted with a gradient of methanol in ethyl acetate). The product containing fractions were concentrated and the residue was trituration with EtOAc/hexanes to give thet title compound as a white solid (1.50 g, 77%).

¹H NMR (500 MHz, CDCl₃) δ ppm 1.83-1.92 (m, 2H) 2.56 (t, J=7.32 Hz, 2H) 2.80 (t, J=7.32 Hz, 2H) 2.98-3.04 (m, 4H) 3.04-3.11 (m, 4H) 5.31 (s, 2H) 7.31 (s, 1H) 7.36 (d, J=7.81 Hz, 1H) 7.86 (d, J=7.81 Hz, 1H)

### Example 431

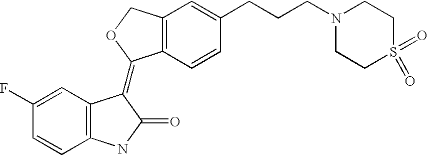

### 3-{5-[3-(1,1-Dioxidothiomorpholin-4-yl)-propyl]-3H-isobenzofuran-1-ylidene}-5-fluoro-1,3-dihydro-indol-2-one

To a stirred solution of 5-fluorooxindole (1.48 g, 9.76 mmol) in anhydrous THF (30 ml) under nitrogen was added 1.0M LiHMDS/THF solution (19.5 ml, 19.5 mmol). The mixture was stirred at room temperature for 10 minutes, and 5-[3-(1,1-dioxidothiomorpholin-4-yl)-propyl]-3H-isobenzofuran-1-one (1.51 g, 4.88 mmol) was added. After stirring at room temperature for 2 hours, the mixture was poured into 30 ml of 2M HCl solution and heated at 50° C. for 30 minutes. The mixture was then poured into 600 ml of water. The resulting solid was filtered, rinsed with water, dried in vacuo to give the title compound as a HCl salt, yellow solid (1.38 g). The filtrate solution was basified with 5M NaOH solution, filtered, washed with water and dried to give a second batch of the title compound as yellow solid (395 mg).

¹H NMR (500 MHz, DMSO-d6) δ ppm 1.75-1.84 (m, 2H) 2.48 (t, J=7.32 Hz, 2H) 2.75 (t, J=7.57 Hz, 2H) 2.84-2.90 (m, 4H) 3.04-3.12 (m, 4H) 5.80 (s, 2H) 6.79 (dd, J=8.30, 4.88 Hz, 1H) 6.89-6.96 (m, 1H) 7.45 (d, J=8.30 Hz, 1H) 7.54 (s, 1H) 7.58 (dd, J=9.52, 2.69 Hz, 1H) 9.54 (d, J=8.30 Hz, 1H) 10.42 (s, 1H)

The following Examples 432-434 were prepared using the experiment procedure described in Example 431, but with the appropriate reagent, reaction conditions and reactant substitutions that will be readily realized by those of ordinary skill in this art without the exercise of undue experimentation.

### Example 432

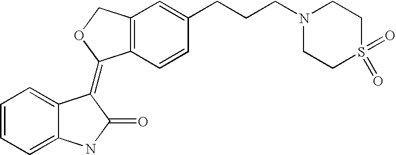

### 3-{5-[3-(1,1-Dioxidothiomorpholin-4-yl)-propyl]-3H-isobenzofuran-1-ylidene}-1,3-dihydro-indol-2-one

1H NMR (500 MHz, DMSO-d6) δ ppm 1.76-1.83 (m, 2H) 2.48 (t, J=7.32 Hz, 2H) 2.75 (t, J=7.81 Hz, 2H) 2.86-2.89 (m, 4H) 3.08 (t, J=4.88 Hz, 4H) 5.77 (s, 2H) 6.82 (d, J=7.32 Hz, 1H) 6.95 (td, J=7.57, 1.46 Hz, 1H) 7.10 (td, J=7.57, 1.46 Hz, 1H) 7.44 (d, J=8.30 Hz, 1H) 7.51 (s, 1H) 7.83 (d, J=7.32 Hz, 1H) 9.55 (d, J=8.30 Hz, 1H) 10.39 (s, 1H)

### Example 433

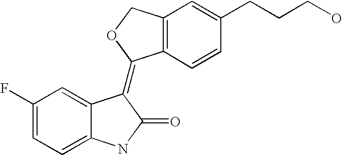

### 5-Fluoro-3-[5-(3-hydroxy-propyl)-3H-isobenzofuran-1-ylidene]-1,3-dihydro-indol-2-one

¹H NMR (500 MHz, DMSO-d6) δ ppm 1.74-1.83 (m, 2H) 2.77 (t, J=7.81 Hz, 2H) 3.42-3.48 (m, 2H) 4.53 (t, J=5.13 Hz, 1H) 5.80 (s, 2H) 6.79 (dd, J=8.30, 4.88 Hz, 1H) 6.89-6.96 (m, 1H) 7.43 (d, J=7.81 Hz, 1H) 7.51 (s, 1H) 7.57 (dd, J=9.76, 2.44 Hz, 1H) 9.54 (d, J=8.30 Hz, 1H) 10.42 (s, 1H)

### Example 434

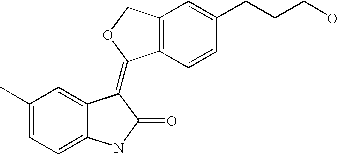

### 3-[5-(3-Hydroxy-propyl)-3H-isobenzofuran-1-ylidene]-5-methyl-1,3-dihydro-indol-2-one

1H NMR (500 MHz, DMSO-d6) δ ppm 1.73-1.82 (m, 2H) 2.30 (s, 3H) 2.76 (t, J=7.81 Hz, 2H) 3.44 (t, J=6.35 Hz, 2H) 3.60 (t, J=6.59 Hz, 1H) 5.77 (s, 2H) 6.70 (d, J=7.81 Hz, 1H) 6.91 (d, J=7.81 Hz, 1H) 7.40 (d, J=8.30 Hz, 1H) 7.48 (s, 1H) 7.67 (s, 1H) 9.55 (d, J=8.30 Hz, 1H) 10.27 (s, 1H)

### Example 435

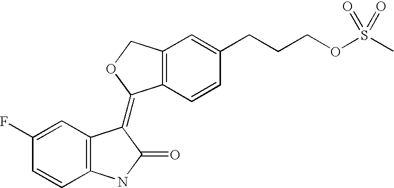

### Methanesulfonic acid 3-[1-(5-fluoro-2-oxo-1,2-dihydro-indol-3-ylidene)-1,3-dihydro-isobenzofuran-5-yl]-propyl ester

To a 0° C. stirred suspension of 5-Fluoro-3-[5-(3-hydroxy-propyl)-3H-isobenzofuran-1-ylidene]-1,3-dihydro-indol-2-one (2.40 g, 7.38 mmol) and triethylamine (1.54 ml, 11.1 mmol) in THF (50 ml), was added methanesulfonyl chloride (0.74 ml, 9.6 mmol). The mixture was stirred for 60 minutes and poured into water, which contained AcOH. The solid was filtered, washed with water and dried under vacuum to afford a crude product. The crude product was purified by silica gel column chromatography, eluted with 5% MeOH in CHCl₃, to give the title compound as a yellow solid (2.93 g, 98%).

¹H NMR (500 MHz, DMSO-d6) δ ppm 2.02-2.09 (m, 2H) 2.84 (t, J=7.32 Hz, 2H) 3.19 (s, 3H) 4.24 (t, J=6.35 Hz, 2H) 5.81 (s, 2H) 6.79 (dd, J=8.30, 4.39 Hz, 1H) 6.90-6.95 (m, 1H) 7.47 (d, J=8.30 Hz, 1H) 7.55 (s, 1H) 7.58 (dd, J=9.76, 2.93 Hz, 1H) 9.56 (d, J=8.30 Hz, 1H) 10.43 (s, 1H)

### Example 436

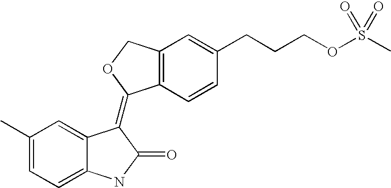

### Methanesulfonic acid 3-[1-(5-methyl-2-oxo-1,2-dihydro-indol-3-ylidene)-1,3-dihydro-isobenzofuran-5-yl]-propyl ester

Experimental procedure similar to Example 435.

1H NMR (500 MHz, DMSO-d6) δ ppm 2.01-2.10 (m, 2H) 2.30 (s, 3H) 2.83 (t, J=7.81 Hz, 2H) 3.19 (s, 3H) 4.24 (t, J=6.35 Hz, 2H) 5.78 (s, 2H) 6.71 (d, J=7.81 Hz, 1H) 6.91 (d, J=7.32 Hz, 1H) 7.44 (d, J=8.30 Hz, 1H) 7.52 (s, 1H) 7.67 (s, 1H) 9.57 (d, J=8.30 Hz, 1H) 10.29 (s, 1H)

### Example 437

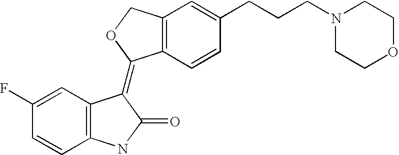

### 5-Fluoro-3-[5-(3-morpholin-4-yl-propyl)-3H-isobenzofuran-1-ylidene]-1,3-dihydro-indol-2-one

A mixture of methanesulfonic acid 3-[1-(5-fluoro-2-oxo-1,2-dihydro-indol-3-ylidene)-1,3-dihydro-isobenzofuran-5-yl]-propyl ester (100 mg, 0.25 mmol) and morpholine (0.5 ml) in DMF (2 ml) was heated at 80° C. for 1 hour. The mixture was cooled, and poured into water (75 ml) with stirring. The solid was filtered, washed with water and dried under vacuum to give the title compound as a yellow solid (88 mg, 89%).

¹H NMR (500 MHz, DMSO-d6) δ ppm 1.76-1.83 (m, 2H) 2.29 (t, J=6.83 Hz, 2H) 2.34 (s, 4H) 2.75 (t, J=7.57 Hz, 2H) 3.57 (t, J=4.39 Hz, 4H) 5.80 (s, 2H) 6.79 (dd, J=8.30, 4.88 Hz, 1H) 6.90-6.95 (m, 1H) 7.44 (d, J=8.30 Hz, 1H) 7.52 (s, 1H) 7.57 (dd, J=9.52, 2.69 Hz, 1H) 9.53 (d, J=8.30 Hz, 1H) 10.42 (s, 1H)

### Example 438

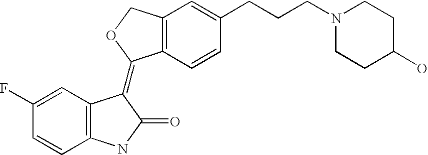

### 5-Fluoro-3-{5-[3-(4-hydroxy-piperidin-1-yl)-propyl]-3H-isobenzofuran-1-ylidene}-1,3-dihydro-indol-2-one

A mixture of methanesulfonic acid 3-[1-(5-fluoro-2-oxo-1,2-dihydro-indol-3-ylidene)-1,3-dihydro-isobenzofuran-5-yl]-propyl ester (70 mg, 0.17 mmol) and 4-hydroxypiperidine (172 mg, 1.7 mmol) in 5 ml of DMF was heated at 85° C. for 1.5 hours. The mixture was poured into 100 ml of water, and stirred at room temp overnight. The yellow precipitates were filtered, washed with water, and dried under vacuum to produce the crude product. The crude product was triturated with chloroform/hexanes to give the title compound as a yellow solid (35 mg, 51%).

1H NMR (500 MHz, DMSO-d6) δ ppm 1.39 (br. s., 2H) 1.63-1.85 (m, 4H) 1.96 (br. s., 2H) 2.26 (br. s., 2H) 2.69 (br. s., 2H) 2.73 (t, J=7.57 Hz, 2H) 3.38-3.47 (m, 1H) 4.47-4.59 (m, 1H) 5.80 (s, 2H) 6.79 (dd, J=8.30, 4.39 Hz, 1H) 6.89-6.96 (m, 1H) 7.43 (d, J=8.30 Hz, 1H) 7.51 (s, 1H) 7.57 (dd, J=9.76, 2.93 Hz, 1H) 9.53 (d, J=8.30 Hz, 1H) 10.42 (s, 1H)

### Example 439

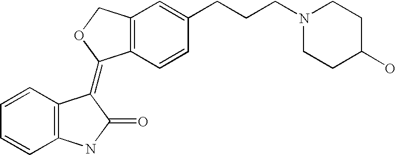

### 3-{5-[3-(4-Hydroxy-piperidin-1-yl)-propyl]-3H-isobenzofuran-1-ylidene}-1,3-dihydro-indol-2-one

Experimental procedure similar to Example 438.

¹H NMR (500 MHz, DMSO-d6) δ ppm 1.33-1.42 (m, 2H) 1.66-1.73 (m, 2H) 1.73-1.81 (m, 2H) 1.96 (t, J=9.76 Hz, 2H) 2.26 (t, J=7.08 Hz, 2H) 2.65-2.75 (m, 4H) 3.39-3.45 (m, 1H) 4.52 (d, J=4.39 Hz, 1H) 5.77 (s, 2H) 6.82 (d, J=7.81 Hz, 1H) 6.95 (t, J=7.57 Hz, 1H) 7.10 (t, J=7.57 Hz, 1H) 7.41 (d, J=8.30 Hz, 1H) 7.49 (s, 1H) 7.82 (d, J=7.32 Hz, 1H) 9.54 (d, J=8.30 Hz, 1H) 10.39 (s, 1H)

### Example 440

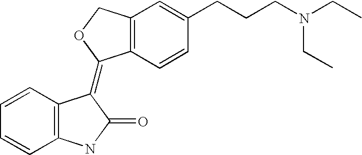

### 3-[5-(3-Diethylamino-propyl)-3H-isobenzofuran-1-ylidene]-1,3-dihydro-indol-2-one

Experimental procedure similar to Example 438.

¹H NMR (500 MHz, DMSO-d6) δ ppm 0.93 (t, J=7.08 Hz, 6H) 1.71-1.78 (m, 2H) 2.40 (t, J=6.83 Hz, 2H) 2.45 (q, J=7.16 Hz, 4H) 2.73 (t, J=7.32 Hz, 2H) 5.77 (s, 2H) 6.82 (d, J=7.81 Hz, 1H) 6.95 (td, J=7.57, 0.98 Hz, 1H) 7.10 (td, J=7.57, 1.46 Hz, 1H) 7.42 (d, J=9.28 Hz, 1H) 7.49 (s, 1H) 7.83 (d, J=7.81 Hz, 1H) 9.54 (d, J=8.30 Hz, 1H) 10.39 (s, 1H)

### Example 441

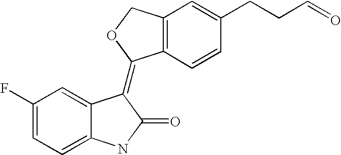

### 3-[1-(5-Fluoro-2-oxo-1,2-dihydro-indol-3-ylidene)-1,3-dihydro-isobenzofuran-5-yl]-propionaldehyde

To a stirred solution of 2M oxalyl chloride/dichloromethane (6.2 ml, 12.4 mmol) and anhydrous dichloromethane (80 ml) at −78° C., was added DMSO (1.8 ml, 25.4 mmol) drop wise. After the mixture was stirred at the same temperature for 10 minutes, a solution of 5-Fluoro-3-[5-(3-hydroxy-propyl)-3H-isobenzofuran-1-ylidene]-1,3-dihydro-indol-2-one (2.0 g, 6.15 mmol) in 10 ml of anhydrous DMSO was added. The mixture was kept at −78° C. for 30 minutes. Triethylamine (4.3 ml, 30.9 mmol) was added. The mixture was allowed to warm to room temperature and poured into 500 ml of water (containing 2 ml of acetic acid). The mixture was stirred at room temperature under reduced pressure for 2 hours. The resulting yellow precipitates were filtered, washed with water, and dried under vacuum to give the title compound as a yellow solid (1.98 g, 100%).

¹H NMR (500 MHz, DMSO-d6) δ ppm 2.88 (t, J=7.57 Hz, 2H) 3.01 (t, J=7.32 Hz, 2H) 5.80 (s, 2H) 6.79 (dd, J=8.30, 4.39 Hz, 1H) 6.90-6.96 (m, 1H) 7.46 (d, J=8.30 Hz, 1H) 7.53 (s, 1H) 7.57 (dd, J=9.76, 2.93 Hz, 1H) 9.54 (d, J=8.30 Hz, 1H) 9.74 (t, J=1.22 Hz, 1H) 10.42 (s, 1H)

MS (ES+): 346.0951 (M+Na⁺)

### Example 442

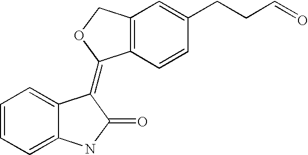

### 3-[1-(2-Oxo-1,2-dihydro-indol-3-ylidene)-1,3-dihydro-isobenzofuran-5-yl]-propionaldehyde

Experimental procedure similar to Example 441.

¹H NMR (500 MHz, DMSO-d6) δ ppm 2.88 (t, J=7.32 Hz, 2H) 3.01 (t, J=7.57 Hz, 2H) 5.77 (s, 2H) 6.82 (d, J=7.32 Hz, 1H) 6.95 (t, J=7.32 Hz, 1H) 7.10 (t, J=7.81 Hz, 1H) 7.45 (d, J=8.30 Hz, 1H) 7.51 (s, 1H) 7.82 (d, J=7.32 Hz, 1H) 9.55 (d, J=8.30 Hz, 1H) 9.74 (t, J=1.22 Hz, 1H) 10.39 (s, 1H)

### Example 443

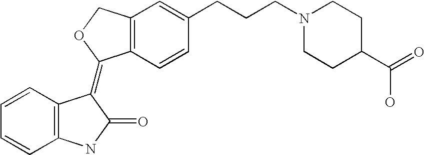

### 1-{3-[1-(2-Oxo-1,2-dihydro-indol-3-ylidene)-1,3-dihydro-isobenzofuran-5-yl]-propyl}-piperidine-4-carboxylic acid

A mixture of 3-[1-(2-Oxo-1,2-dihydro-indol-3-ylidene)-1,3-dihydro-isobenzofuran-5-yl]-propionaldehyde (70 mg, 0.23 mmol), isonipecotic acid (120 mg, 0.92 mmol), acetic acid (2 drops) and sodium cyanoborohydride (50 mg, 0.80 mmol) in methanol (6 ml) was stirred at room temperature for 1 hour. The mixture was poured into 70 ml of water, and adjusted to pH about 7 with saturated NaHCO₃ solution and AcOH. The resulting precipitates were filtered, washed with water and dried under vacuum to give the title compound as yellow powder (63 mg, 66%).

¹H NMR (500 MHz, DMSO-d6) δ ppm 1.48-1.60 (m, 2H) 1.73-1.82 (m, 4H) 1.92 (t, J=10.74 Hz, 2H) 2.12-2.22 (m, 1H) 2.28 (t, J=7.08 Hz, 2H) 2.73 (t, J=7.57 Hz, 2H) 2.78 (d, J=11.72 Hz, 2H) 5.77 (s, 2H) 6.82 (d, J=7.81 Hz, 1H) 6.95 (t, J=7.08 Hz, 1H) 7.10 (t, J=7.32 Hz, 1H) 7.41 (d, J=8.30 Hz, 1H) 7.49 (s, 1H) 7.82 (d, J=7.32 Hz, 1H) 9.54 (d, J=8.30 Hz, 1H) 10.39 (s, 1H)

The following Examples 444-467 were prepared using the experiment procedure described in Example 443, but with the appropriate reagent, reaction conditions and reactant substitutions that will be readily realized by those of ordinary skill in this art without the exercise of undue experimentation.

### Example 444

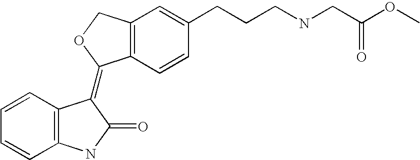

### {3-[1-(2-Oxo-1,2-dihydro-indol-3-ylidene)-1,3-dihydro-isobenzofuran-5-yl]-propylamino}-acetic acid methyl ester

¹H NMR (500 MHz, DMSO-d6) δ ppm 1.71-1.81 (m, 2H) 2.02 (br s, 1H) 2.54 (t, J=6.83 Hz, 2H) 2.76 (t, J=7.32 Hz, 2H) 3.33 (s, 2H) 3.62 (s, 3H) 5.77 (s, 2H) 6.82 (d, J=7.32 Hz, 1H) 6.95 (td, J=7.57, 1.46 Hz, 1H) 7.10 (td, J=7.57, 1.46 Hz, 1H) 7.41 (d, J=8.30 Hz, 1H) 7.49 (s, 1H) 7.82 (d, J=7.32 Hz, 1H) 9.55 (d, J=7.81 Hz, 1H) 10.39 (s, 1H)

### Example 445

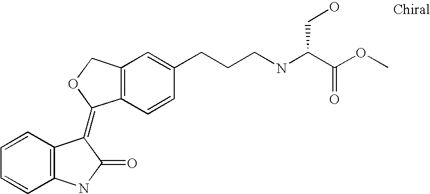

### 3-Hydroxy-2-{3-[1-(2-oxo-1,2-dihydro-indol-3-ylidene)-1,3-dihydro-isobenzofuran-5-yl]-propylamino}-propionic acid methyl ester

¹H NMR (500 MHz, DMSO-d6) δ ppm 1.70-1.79 (m, 2H) 1.99 (br. s., 1H) 2.40-2.48 (m, 1H) 2.55-2.63 (m, 1H) 2.72-2.79 (m, 2H) 3.26 (br. s., 1H) 3.52-3.58 (m, 2H) 3.62 (s, 3H) 4.81 (t, J=5.86 Hz, 1H) 5.77 (s, 2H) 6.82 (d, J=7.32 Hz, 1H) 6.95 (t, J=7.57 Hz, 1H) 7.10 (t, J=7.57 Hz, 1H) 7.41 (d, J=8.79 Hz, 1H) 7.48 (s, 1H) 7.82 (d, J=7.81 Hz, 1H) 9.54 (d, J=8.30 Hz, 1H) 10.39 (s, 1H)

### Example 446

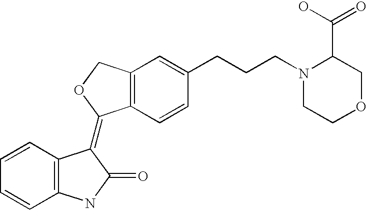

### 4-{3-[1-(2-Oxo-1,2-dihydro-indol-3-ylidene)-1,3-dihydro-isobenzofuran-5-yl]-propyl}-morpholine-3-carboxylic acid

¹H NMR (500 MHz, DMSO-d6) δ ppm 1.77-1.87 (m, 2H) 2.35-2.42 (m, 1H) 2.48-2.56 (m, 1H) 2.68-2.81 (m, 3H) 3.04-3.10 (m, 1H) 3.19 (t, J=4.39 Hz, 1H) 3.58-3.68 (m, 2H) 3.69-3.77 (m, 2H) 5.77 (s, 2H) 6.82 (d, J=7.32 Hz, 1H) 6.95 (td, J=7.57, 0.98 Hz, 1H) 7.10 (td, J=7.57, 0.98 Hz, 1H) 7.42 (d, J=8.30 Hz, 1H) 7.50 (s, 1H) 7.83 (d, J=7.32 Hz, 1H) 9.54 (d, J=8.30 Hz, 1H) 10.39 (s, 1H)

### Example 447

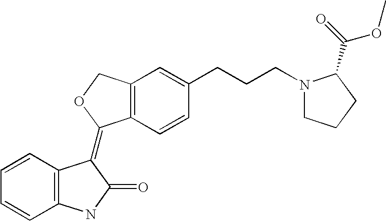

### 1-{3-[1-(2-Oxo-1,2-dihydro-indol-3-ylidene)-1,3-dihydro-isobenzofuran-5-yl]-propyl}-pyrrolidine-2-carboxylic acid methyl ester

### Example 448

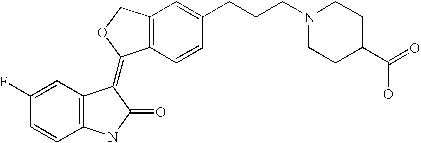

### 1-{3-[1-(5-Fluoro-2-oxo-1,2-dihydro-indol-3-ylidene)-1,3-dihydro-isobenzofuran-5-yl]-propyl}-piperidine-4-carboxylic acid

MS (ES+): 437.1812 (MH⁺)

MS (ES−): 435.1787 (M−H)

### Example 449

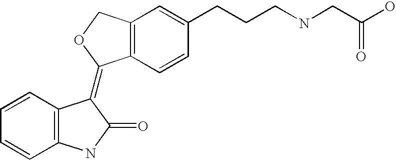

### {3-[1-(2-Oxo-1,2-dihydro-indol-3-ylidene)-1,3-dihydro-isobenzofuran-5-yl]-propylamino}-acetic acid

¹H NMR (500 MHz, DMSO-d6) δ ppm 1.81 (br s, 1H) 1.89-1.97 (m, 2H) 2.77 (t, J=7.57 Hz, 2H) 2.83 (t, J=7.81 Hz, 2H) 3.13 (br. s., 2H) 5.78 (s, 2H) 6.83 (d, J=7.81 Hz, 1H) 6.96 (t, J=7.81 Hz, 1H) 7.10 (t, J=7.57 Hz, 1H) 7.43 (d, J=8.30 Hz, 1H) 7.50 (s, 1H) 7.83 (d, J=7.81 Hz, 1H) 9.57 (d, J=8.30 Hz, 1H) 10.41 (s, 1H)

### Example 450

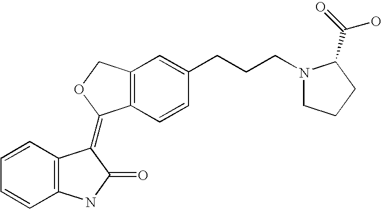

### 1-{3-[1-(2-Oxo-1,2-dihydro-indol-3-ylidene)-1,3-dihydro-isobenzofuran-5-yl]-propyl}-pyrrolidine-2-carboxylic acid

### Example 451

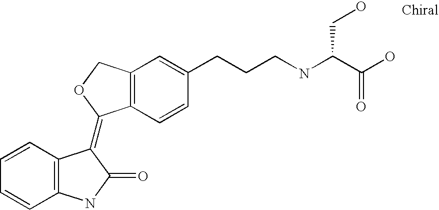

### 3-Hydroxy-2-{3-[1-(2-oxo-1,2-dihydro-indol-3-ylidene)-1,3-dihydro-isobenzofuran-5-yl]-propylamino}-propionic acid

MS (ES+): 395.1596 (MH⁺)

MS (ES−): 393.1483 (M−H)

### Example 452

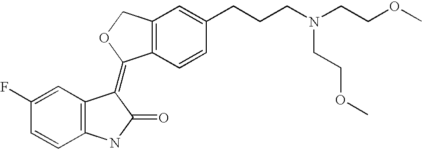

### 3-(5-{3-[Bis-(2-methoxy-ethyl)-amino]-propyl}-3H-isobenzofuran-1-ylidene)-5-fluoro-1,3-dihydro-indol-2-one

¹H NMR (500 MHz, DMSO-d6) δ ppm 1.71-1.77 (m, 2H) 2.50 (t, J=5.37 Hz, 2H) 2.61 (t, J=6.10 Hz, 4H) 2.73 (t, J=7.81 Hz, 2H) 3.22 (s, 6H) 3.36 (t, J=6.35 Hz, 4H) 5.80 (s, 2H) 6.79 (dd, J=8.54, 4.64 Hz, 1H) 6.90-6.95 (m, 1H) 7.43 (d, J=8.30 Hz, 1H) 7.51 (s, 1H) 7.58 (dd, J=9.76, 2.44 Hz, 1H) 9.54 (d, J=8.30 Hz, 1H) 10.42 (s, 1H)

### Example 453

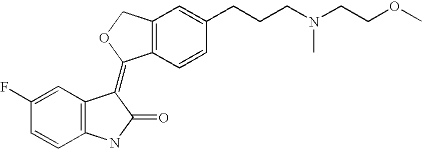

### 5-Fluoro-3-(5-{3-[(2-methoxy-ethyl)-methyl-amino]-propyl}-3H-isobenzofuran-1-ylidene)-1,3-dihydro-indol-2-one

¹H NMR (500 MHz, DMSO-d6) δ ppm 1.72-1.80 (m, 2H) 2.19 (s, 3H) 2.36 (t, J=6.83 Hz, 2H) 2.49 (t, J=5.86 Hz, 2H) 2.73 (t, J=7.32 Hz, 2H) 3.23 (s, 3H) 3.40 (t, J=5.86 Hz, 2H) 5.80 (s, 2H) 6.79 (dd, J=8.30, 4.88 Hz, 1H) 6.92 (td, J=9.03, 2.93 Hz, 1H) 7.44 (d, J=8.30 Hz, 1H) 7.52 (s, 1H) 7.58 (dd, J=9.76, 2.93 Hz, 1H) 9.54 (d, J=8.30 Hz, 1H) 10.42 (s, 1H)

### Example 454

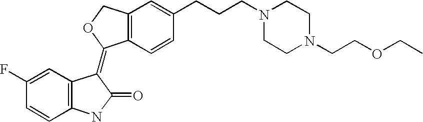

### 3-(5-{3-[4-(2-Ethoxy-ethyl)-piperazin-1-yl]-propyl}-3H-isobenzofuran-1-ylidene)-5-fluoro-1,3-dihydro-indol-2-one

¹H NMR (500 MHz, DMSO-d6) δ ppm 1.09 (t, J=7.32 Hz, 3H) 1.73-1.81 (m, 2H) 2.27 (t, J=6.83 Hz, 2H) 2.3-2.5 (br s, 8H) 2.43 (t, J=6.10 Hz, 2H) 2.73 (t, J=7.81 Hz, 2H) 3.40 (q, J=7.00 Hz, 2H) 3.44 (t, J=6.10 Hz, 2H) 5.80 (s, 2H) 6.79 (dd, J=8.30, 4.39 Hz, 1H) 6.90-6.95 (m, 1H) 7.43 (d, J=8.30 Hz, 1H) 7.51 (s, 1H) 7.57 (dd, J=9.52, 2.69 Hz, 1H) 9.53 (d, J=7.81 Hz, 1H) 10.42 (s, 1H)

### Example 455

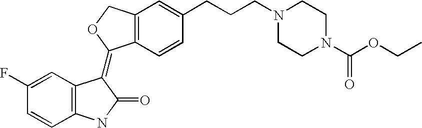

### 4-{3-[1-(5-Fluoro-2-oxo-1,2-dihydro-indol-3-ylidene)-1,3-dihydro-isobenzofuran-5-yl]-propyl}-piperazine-1-carboxylic acid ethyl ester

¹H NMR (500 MHz, DMSO-d6) δ ppm 1.17 (t, J=6.83 Hz, 3H) 1.76-1.84 (m, 2H) 2.32 (t, J=7.08 Hz, 6H) 2.75 (t, J=7.32 Hz, 2H) 3.35 (br. s., 4H) 4.02 (q, J=7.32 Hz, 2H) 5.80 (s, 2H) 6.79 (dd, J=8.54, 4.64 Hz, 1H) 6.90-6.95 (m, 1H) 7.44 (d, J=8.30 Hz, 1H) 7.52 (s, 1H) 7.58 (dd, J=9.52, 2.69 Hz, 1H) 9.53 (d, J=8.30 Hz, 1H) 10.42 (s, 1H)

### Example 456

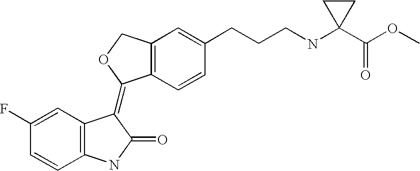

### 1-{3-[1-(5-Fluoro-2-oxo-1,2-dihydro-indol-3-ylidene)-1,3-dihydro-isobenzofuran-5-yl]-propylamino}-cyclopropanecarboxylic acid methyl ester

¹H NMR (500 MHz, DMSO-d6) δ ppm 0.89 (q, J=3.74 Hz, 2H) 1.12 (q, J=3.58 Hz, 2H) 1.67-1.76 (m, 2H) 2.65 (t, J=6.59 Hz, 2H) 2.75 (t, J=7.32 Hz, 2H) 3.59 (s, 3H) 5.80 (s, 2H) 6.79 (dd, J=8.54, 4.64 Hz, 1H) 6.90-6.95 (m, 1H) 7.42 (d, J=8.30 Hz, 1H) 7.50 (s, 1H) 7.57 (dd, J=9.76, 2.44 Hz, 1H) 9.5 (d, J=8.30 Hz, 1H) 10.42 (s, 1H)

### Example 457

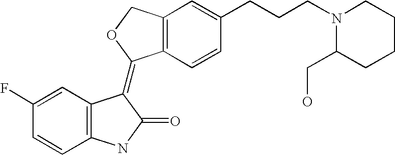

### 5-Fluoro-3-{5-[3-(2-hydroxymethyl-piperidin-1-yl)-propyl]-3H-isobenzofuran-1-ylidene}-1,3-dihydro-indol-2-one

¹H NMR (500 MHz, DMSO-d6) δ ppm 1.18-1.32 (m, 2H) 1.35-1.43 (m, 1H) 1.47-1.54 (m, 1H) 1.58-1.67 (m, 2H) 1.73-1.82 (m, 2H) 2.15 (t, J=9.28 Hz, 1H) 2.23 (br. s., 1H) 2.35-2.42 (m, 1H) 2.63-2.80 (m, 4H) 3.29-3.32 (br s, 1H) 3.51 (dd, J=10.25, 3.42 Hz, 1H) 4.33 (br. s., 1H) 5.80 (s, 2H) 6.79 (dd, J=8.30, 4.39 Hz, 1H) 6.89-6.95 (m, 1H) 7.43 (d, J=8.30 Hz, 1H) 7.52 (s, 1H) 7.58 (dd, J=9.52, 2.69 Hz, 1H) 9.53 (d, J=8.30 Hz, 1H) 10.42 (s, 1H)

### Example 458

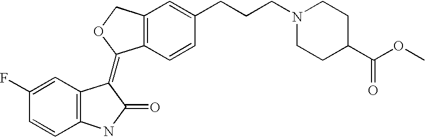

### 1-{3-[1-(5-Fluoro-2-oxo-1,2-dihydro-indol-3-ylidene)-1,3-dihydro-isobenzofuran-5-yl]-propyl}-piperidine-4-carboxylic acid methyl ester

MS (ES−): 449.4623 (M−H)

### Example 459

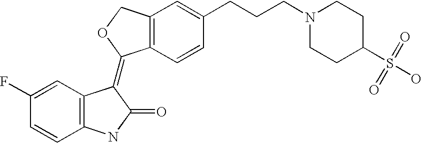

### 1-{3-[1-(5-Fluoro-2-oxo-1,2-dihydro-indol-3-ylidene)-1,3-dihydro-isobenzofuran-5-yl]-propyl}-piperidine-4-sulfonic acid

MS (ES+): 473.1855 (MH⁺), 495.1568 (M+Na⁺)

MS (ES−): 471.1337 (M−H)

### Example 460

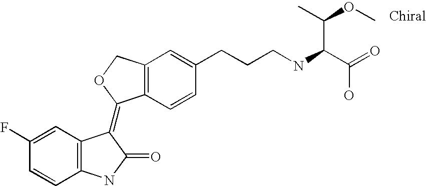

### 2-{3-[1-(5-Fluoro-2-oxo-1,2-dihydro-indol-3-ylidene)-1,3-dihydro-isobenzofuran-5-yl]-propylamino}-3-methoxy-butyric acid

¹H NMR (500 MHz, DMSO-d6) δ ppm 1.20 (d, J=6.35 Hz, 3H) 1.85-2.01 (m, 2H) 2.72-2.87 (m, 4H) 3.04 (d, J=5.86 Hz, 1H) 3.24 (s, 3H) 3.56-3.63 (m, 1H) 5.81 (s, 2H) 6.79 (dd, J=8.30, 4.39 Hz, 1H) 6.90-6.95 (m, 1H) 7.44 (d, J=8.30 Hz, 1H) 7.52 (s, 1H) 7.57 (dd, J=9.76, 2.93 Hz, 1H) 9.55 (d, J=8.30 Hz, 1H) 10.43 (s, 1H)

### Example 461

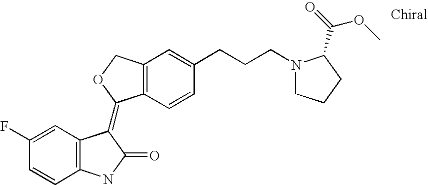

### 1-{3-[1-(5-Fluoro-2-oxo-1,2-dihydro-indol-3-ylidene)-1,3-dihydro-isobenzofuran-5-yl]-propyl}-pyrrolidine-2-carboxylic acid methyl ester

¹H NMR (500 MHz, DMSO-d6) δ ppm 1.69-1.85 (m, 5H) 1.98-2.08 (m, 1H) 2.30-2.44 (m, 2H) 2.60-2.68 (m, 1H) 2.69-2.82 (m, 2H) 2.99-3.05 (m, 1H) 3.17 (dd, J=8.79, 4.88 Hz, 1H) 3.60 (s, 3H) 5.80 (s, 2H) 6.79 (dd, J=8.30, 4.39 Hz, 1H) 6.90-6.96 (m, 1H) 7.42 (d, J=8.30 Hz, 1H) 7.51 (s, 1H) 7.58 (dd, J=9.76, 2.44 Hz, 1H) 9.53 (d, J=7.81 Hz, 1H) 10.42 (s, 1H)

### Example 462

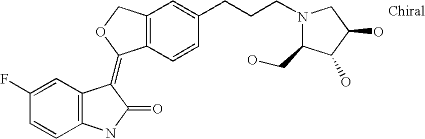

### 3-{5-[3-(3,4-Dihydroxy-2-hydroxymethyl-pyrrolidin-1-yl)-propyl]-3H-isobenzofuran-1-ylidene}-5-fluoro-1,3-dihydro-indol-2-one

MS (ES+): 441.1705 (MH⁺)

MS (ES−): 439.1664 (M−H)

### Example 463

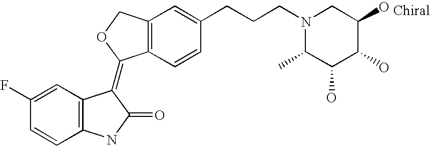

### 5-Fluoro-3-{5-[3-(3,4,5-trihydroxy-2-methyl-piperidin-1-yl)-propyl]-3H-isobenzofuran-1-ylidene}-1,3-dihydro-indol-2-one

MS (ES+): 455.1888 (MH⁺), 477.1709 (M+Na⁺)

MS (ES−): 453.1854 (M−H)

### Example 464

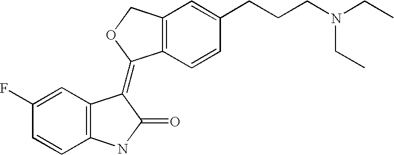

### 3-[5-(3-Diethylamino-propyl)-3H-isobenzofuran-1-ylidene]-5-fluoro-1,3-dihydro-indol-2-one

¹H NMR (500 MHz, DMSO-d6) δ ppm 0.98 (br. s., 6H) 1.77 (br. s., 2H) 2.45 (br s, 6H) 2.74 (t, J=7.81 Hz, 2H) 5.80 (s, 2H) 6.79 (dd, J=8.54, 4.64 Hz, 1H) 6.90-6.96 (m, 1H) 7.44 (d, J=8.30 Hz, 1H) 7.52 (s, 1H) 7.58 (dd, J=9.76, 2.93 Hz, 1H) 9.54 (d, J=8.30 Hz, 1H) 10.42 (s, 1H)

### Example 465

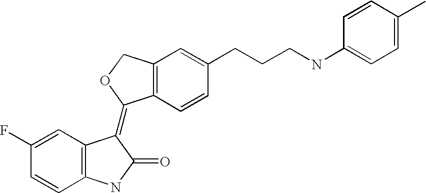

### 5-Fluoro-3-[5-(3-p-tolylamino-propyl)-3H-isobenzofuran-1-ylidene]-1,3-dihydro-indol-2-one

1H NMR (500 MHz, DMSO-d6) δ ppm 1.86-1.93 (m, 2H) 2.14 (s, 3H) 2.83 (t, J=7.81 Hz, 2H) 3.01 (q, J=6.83 Hz, 2H) 5.39 (t, J=5.61 Hz, 1H) 5.81 (s, 2H) 6.47 (d, J=8.30 Hz, 2H) 6.79 (dd, J=8.30, 4.88 Hz, 1H) 6.88 (d, J=8.30 Hz, 2H) 6.90-6.96 (m, 1H) 7.45 (d, J=8.30 Hz, 1H) 7.53 (s, 1H) 7.58 (dd, J=9.76, 2.44 Hz, 1H) 9.55 (d, J=7.81 Hz, 1H) 10.42 (s, 1H)

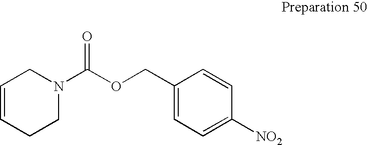

### Preparation of 3,6-dihydro-2H-pyridine-1-carboxylic acid 4-nitro-benzyl ester

To a stirred mixture of tetrahydropyridine (5.0 g, 60.1 mmol) and 25 ml of 10% Na₂CO₃ aqueous solution at 0° C. was added 4-nitrophenyl chloroformate (11.9 g, 55 mmol) in portions over 1.5 hours. The resulting suspension was continuously stirred at 0° C. for 3 hours. The mixture was then diluted with 50 ml of brine, filtered, washed with water, and dried in vacuum to give crude product as a yellow powder. Recrystallization with EtOAc/hexanes afforded 3,6-dihydro-2H-pyridine-1-carboxylic acid 4-nitro-benzyl ester as white crystals (13.43 g, 93%).

¹H NMR (500 MHz, CDCl₃) δ ppm 2.20 (s, 2H) 3.61 (s, 2H) 4.01 (s, 2H) 5.27 (s, 2H) 5.69 (br. s., 1H) 5.88 (br. s., 1H) 7.54 (d, J=8.79 Hz, 2H) 8.24 (d, J=8.79 Hz, 2H)

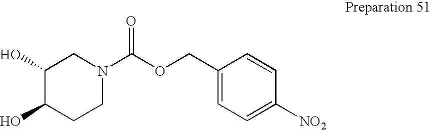

### Preparation of trans-3,4-dihydroxy-piperidine-1-carboxylic acid 4-nitro-benzyl ester

To a stirred solution of 3,6-dihydro-2H-pyridine-1-carboxylic acid 4-nitro-benzyl ester (2.0 g, 7.63 mmol) in 8 ml of formic acid, was added 2 ml of 30% H₂O₂ solution drop wise. The mixture was heated at 45° C. for 1 hour, and evaporated to remove formic acid. The residue was diluted with 15 ml of water, cooled to 0° C. and basified with 5M NaOH solution to pH>10. After stirred at room temperature for 30 minutes, the mixture was extracted with ethyl acetate (3×75 ml). Organic layers were combined, washed with brine (100 ml), dried over Na₂SO₄, and evaporated to produce a residual oil. Recrystallization of the oil with EtOAc/hexanes led to trans-3,4-dihydroxy-piperidine-1-carboxylic acid 4-nitro-benzyl ester as white crystals (1.84 g, 81%).

¹H NMR (500 MHz, CDCl₃) δ ppm 1.48-1.65 (m, 2H) 1.99-2.06 (m, 1H) 2.29 (s, 1H) 2.84 (br. s., 1H) 2.90-3.10 (m, 1H) 3.49 (br. s., 1H) 3.60 (br. s., 1H) 4.01-4.18 (m, J=7.32 Hz, 1H) 4.24 (d, J=11.72 Hz, 1H) 5.24 (d, J=8.79 Hz, 2H) 7.53 (d, J=8.79 Hz, 2H) 8.23-8.26 (m, 2H)

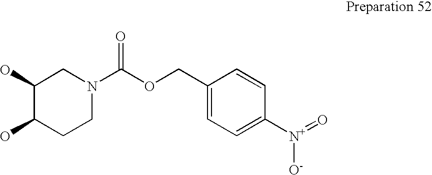

### Preparation of cis-3,4-dihydroxy-piperidine-1-carboxylic acid 4-nitro-benzyl ester

A mixture of 3,6-dihydro-2H-pyridine-1-carboxylic acid 4-nitro-benzyl ester (2.0 g, 7.62 mmol), 4-methylmorpholine N-oxide (1.34 g, 11.4 mmol) and 2.5% Osmium tetroxide/t-butanol solution (1.9 ml, 0.15 mmol) in 1:1 acetone/water solution (12 ml) was sealed and stirred at room temperature for 5 days. The mixture was poured into 100 ml of Na₂S₂O₅ aqueous solution, which was pre-cooled with ice). The precipitates were filtered, washed with water, and dried under vacuum to give the crude product. The crude product was dissolved in EtOAc (100 ml), and then an insoluble dark material was removed through filtration. The filtrate was evaporated, and the obtained residue was recrystallized from EtOAc/hexane to give cis-3,4-dihydroxy-piperidine-1-carboxylic acid 4-nitro-benzyl ester as grey solids. (1.3 g, 58%).

¹H NMR (500 MHz, CDCl₃) δ ppm 1.70-1.80 (m, 6.35 Hz, 1H) 1.86-1.94 (m, 1H) 2.11 (br. s., 2H) 3.34 (br. s., 1H) 3.50 (br. s., 1H) 3.73 (br. s., 2H) 3.85 (br. s., 1H) 3.93 (s, 1H) 5.21-5.31 (m, 2H) 7.54 (d, J=8.30 Hz, 2H) 8.24 (d, J=8.79 Hz, 2H)

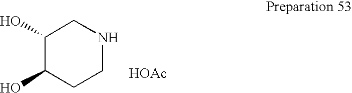

### Preparation of trans-piperidine-3,4-diol

A mixture of trans-3,4-dihydroxy-piperidine-1-carboxylic acid 4-nitro-benzyl ester (1.0 g), 100 mg of 10% Pd/C and 1 ml of AcOH in 20 ml of methanol was hydrogenated under H₂ (45 psi) for 15 min. The mixture was filtered through celite, and concentrated to give light yellow oil. The oil was dissolved in 3 ml of methanol, and diluted with 70 ml of ether with stirring. The upper clear solution was then decanted off. This procedure was repeated one more time. The obtained residue was evaporated and dried under vacuum to give trans-piperidine-3,4-diol AcOH salt as colorless oil (0.53 g, 88%).

MS (ES+): 118.0791 (MH⁺)

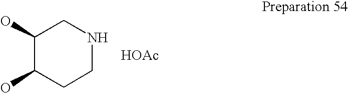

### Preparation of cis-piperidine-3,4-diol

A mixture of cis-3,4-dihydroxy-piperidine-1-carboxylic acid 4-nitro-benzyl ester (1.0 g), 100 mg of 10% Pd/C and 1 ml of AcOH in 20 ml of methanol was hydrogenated under H₂ (45 psi) for 30 min. The mixture was filtered through celite, and concentrated and diluted with 70 ml of ether with stirring. The upper clear solution was then decanted off. This procedure was repeated one more time. The obtained residue was evaporated and dried under vacuum to give cis-piperidine-3,4-diol AcOH salt as a white powder (558 mg, 93%).

¹H NMR (500 MHz, d4-CH₃OH) δ ppm 1.76-1.85 (m, 1H) 1.91 (s, 3H) 1.96-2.05 (m, 1H) 2.91-2.98 (m, 1H) 3.03 (dd, J=12.94, 2.20 Hz, 1H) 3.16-3.24 (m, 2H) 3.78-3.84 (m, 1H) 3.86-3.91 (m, 1H)

### Example 466

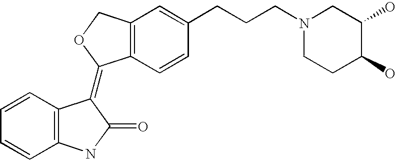

### trans-3-{5-[3-(3,4-Dihydroxy-piperidin-1-yl)-propyl]-3H-isobenzofuran-1-ylidene}-1,3-dihydro-indol-2-one

¹H NMR (500 MHz, DMSO-d6) δ ppm 1.30-1.41 (m, 1H) 1.67-1.81 (m, 4H) 1.86 (t, J=10.74 Hz, 1H) 2.24-2.32 (m, 2H) 2.67-2.75 (m, 3H) 2.81 (dd, J=10.98, 2.68 Hz, 1H) 3.06-3.13 (m, 1H) 3.19-3.25 (m, 1H) 4.63 (d, J=4.39 Hz, 1H) 4.66 (d, J=4.88 Hz, 1H) 5.77 (s, 2H) 6.82 (d, J=7.32 Hz, 1H) 6.95 (td, J=7.57, 0.98 Hz, 1H) 7.10 (td, J=7.69, 1.22 Hz, 1H) 7.41 (d, J=8.30 Hz, 1H) 7.49 (s, 1H) 7.83 (d, J=7.81 Hz, 1H) 9.54 (d, J=8.30 Hz, 1H) 10.39 (s, 1H)

### Example 467

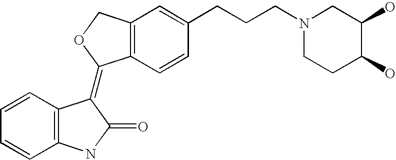

### cis-3-{5-[3-(3,4-Dihydroxy-piperidin-1-yl)-propyl]-3H-isobenzofuran-1-ylidene}-1,3-dihydro-indol-2-one

¹H NMR (500 MHz, DMSO-d6) δ ppm 1.50-1.57 (m, 1H) 1.62-1.69 (m, 1H) 1.73-1.82 (m, 2H) 2.22-2.32 (m, 5H) 2.36 (br. s., 1H) 2.73 (t, J=7.57 Hz, 2H) 3.46-3.53 (m, 1H) 3.62 (br. s., 1H) 4.21 (d, J=3.42 Hz, 1H) 4.29 (d, J=3.91 Hz, 1H) 5.77 (s, 2H) 6.82 (d, J=7.81 Hz, 1H) 6.95 (t, J=7.81 Hz, 1H) 7.10 (td, J=7.57, 1.46 Hz, 1H) 7.42 (d, J=8.30 Hz, 1H) 7.49 (s, 1H) 7.83 (d, J=7.81 Hz, 1H) 9.54 (d, J=8.30 Hz, 1H) 10.39 (s, 1H)

### Example 468

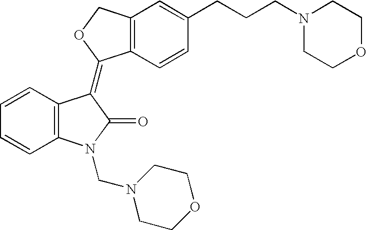

### 1-Morpholin-4-ylmethyl-3-[5-(3-morpholin-4-yl-propyl)-3H-isobenzofuran-1-ylidene]-1,3-dihydro-indol-2-one

A mixture of 3-[5-(3-morpholin-4-yl-propyl)-3H-isobenzofuran-1-ylidene]-1,3-dihydro-indol-2-one (200 mg, 0.53 mmol), paraformaldehyde (80 mg, 2.7 mmol) and morpholine (113 mg, 1.3 mmol) in 10 ml of anhydrous ethanol was heated at 80° C. for 16 hours. The mixture was concentrated and purified through silica gel column chromatography by elution with a gradient of MeOH in CHCl₃. Triturating the resulting crude with chloroform/hexanes gave the title compound as a yellow powder.

¹H NMR (500 MHz, DMSO-d6) δ ppm 1.76-1.83 (m, 2H) 2.29 (t, J=6.83 Hz, 2H) 2.35 (br. s., 4H) 2.55 (t, J=4.88 Hz, 4H) 2.76 (t, J=7.32 Hz, 2H) 3.54 (t, J=4.39 Hz, 4H) 3.57 (t, J=4.39 Hz, 4H) 4.53 (s, 2H) 5.80 (s, 2H) 7.04 (td, J=7.44, 1.22 Hz, 1H) 7.14 (d, J=7.81 Hz, 1H) 7.19 (td, J=7.57, 0.98 Hz, 1H) 7.44 (d, J=8.30 Hz, 1H) 7.52 (s, 1H) 7.90 (d, J=6.83 Hz, 1H) 9.56 (d, J=8.30 Hz, 1H)

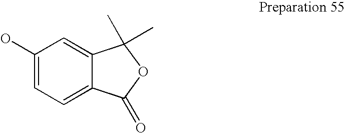

### Preparation of 5-hydroxy-3,3-dimethyl-3H-isobenzofuran-1-one

To a solution of 5-methoxy-3,3-dimethyl-3H-isobenzofuran-1-one (2.2 g, 11.5 mmol) in 1,2-dichloroethane (18 ml) at 0° C. was added 1.0M boron tribromide/dichloromethane (25.2 ml) over several minutes. The reaction is stirred for 10 minutes at 0° C. and then 17 hours at room temperature. The solution was slowly poured into an ice water (300 ml) and a white precipitate separated. The mixture is extracted with EtOAc and the organic layers were washed with saturated NaHCO₃, dilute HCl, brine and then evaporated to a grey solid. The solid is triturated with hexane (50 ml) to give 5-hydroxy-3,3-dimethyl-3H-isobenzofuran-1-one as a grey solid (1.94 g, 95%).

¹H NMR (500 MHz, DMSO-d6) δ ppm 1.56 (s, 6H) 6.91-6.94 (m, 1H) 6.95 (d, J=1.95 Hz, 1H) 7.60 (d, J=8.30 Hz, 1H) 10.67 (s, 1H).

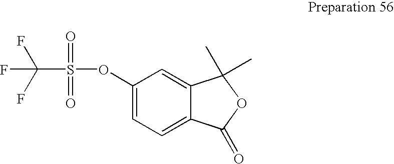

### Preparation of trifluoromethanesulfonic acid 3,3-dimethyl-1-oxo-1,3-dihydro-isobenzofuran-5-yl ester

To the slurry of 5-hydroxy-3,3-dimethyl-3H-isobenzofuran-1-one (1.49 g, 8.4 mmol) in dichloromethane (50 ml) was added triethylamine (2.3 ml, 16.7 mmol) to give a clear solution. Upon cooling to −50° C. was added trifluoromethanesulfonic anhydride (1.6 ml, 9.6 mmol) over 3 min to give an orange-brown solution. After 20 min at −50° C. the solution was poured into a mixture of EtOAc (350 ml) and 0.5% HCl (100 ml). The aqueous layer is removed and the organic layer was washed with 0.5% HCl, H₂O, diluted NaOH, brine, 0.1% HCl, brine, dried over anhydrous Na₂SO₄, and then evaporated to oil. The oil was passed through a small pad of silica gel eluting with CHCl₃, and then evaporated. The oil was then crystallized from hexane to give trifluoromethanesulfonic acid 3,3-dimethyl-1-oxo-1,3-dihydro-isobenzofuran-5-yl ester as an off-white solid (2.45 g, 95%).

¹H NMR (500 MHz, CDCl₃) δ ppm 1.70 (s, 6H) 7.32 (d, J=1.46 Hz, 1H) 7.42 (dd, J=8.30, 2.44 Hz, 1H) 7.97 (d, J=8.30 Hz, 1H).

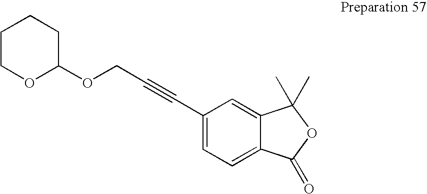

### Preparation of 3,3-dimethyl-5-[3-(tetrahydro-pyran-2-yloxy)-prop-1-ynyl]-3H-isobenzofuran-1-one

A solution containing trifluoromethanesulfonic acid 3,3-dimethyl-1-oxo-1,3-dihydro-isobenzofuran-5-yl ester (1.40 g, 4.52 mmol), tetrahydro-2-(2-propynyloxy)-2H-pyran (1.9 ml, 13.6 mmol), and triethylamine (1.3 ml, 9.0 mmol) in DMF (15.0 ml) at room temperature was degassed with argon. Then copper(I) iodide (0.26 g, 1.36 mmol) and tetrakis(triphenylphosphine)palladium (0.39 g, 0.34 mmol) were added and the reaction was stirred at room temperature for 20 hours. The reaction was added to methanol (200 ml) and H₂O (5 ml) and the dark precipitate removed by filtration. The solution was evaporated, dissolved in EtOAc, and washed with H₂O, diluted HCl, and H₂O, brine, dried over anhydrous Na₂SO₄. Removal of the solvent led to a dark oil. The oil was then rotary evaporated with xylenes and then toluene to remove excess tetrahydro-2-(2-propynyloxy)-2H-pyran. The oil was chromatographed eluting with gradient 20% to 30% EtOAc in hexane to give orange oil. The oil was triturated with hexane to give the title compound as a light yellow solid (1.27 g, 94%).

¹H NMR (500 MHz, CDCl₃) δ ppm 1.54-1.58 (m, 2H) 1.63-1.71 (m, 8H) 1.75-1.90 (m, 2H) 3.56-3.61 (m, 1H) 3.86-3.92 (m, 1H) 4.46-4.57 (m, 2H) 4.89 (t, J=3.42 Hz, 1H) 7.47 (s, 1H) 7.56 (dd, J=7.81, 0.98 Hz, 1H) 7.80 (d, J=7.81 Hz, 1H).

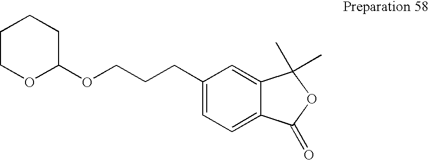

### Preparation of 3,3-dimethyl-5-[3-(tetrahydro-pyran-2-yloxy)-prop-1-ynyl]-3H-isobenzofuran-1-one

A mixture of 3,3-dimethyl-5-[3-(tetrahydro-pyran-2-yloxy)-prop-1-ynyl]-3H-isobenzofuran-1-one (1.27 g, 4.21 mmol) and 10% palladium on carbon (300 mg) in MeOH (50 ml) was shaken under 55 psi of hydrogen for 16 hours. Upon checking the reaction by TLC, additional 10% palladium on carbon (170 mg) was added and the reaction continued for 68 hours. The catalyst was removed by filtration through celite and rinsed with MeOH and EtOAc. The combined filtrates were evaporated to oil. The oil was again dissolved in methanol (40 ml) with 10% palladium on carbon (400 mg) and shaken under 55 psi of hydrogen for 21 hours. The catalyst was removed by filtration through celite and rinsed with MeOH and EtOAc and the combined filtrates were evaporated to oil. The oil was chromatographed eluting with gradient 20% to 30% EtOAc in hexane to give the title compound as a light orange oil (1.13 g, 88%).

¹H NMR (500 MHz, CDCl₃) δ ppm 1.50-1.59 (m, 4H) 1.63 (s, 6H) 1.69-1.75 (m, 1H) 1.79-1.85 (m, 1H) 1.92-1.98 (m, 2H) 2.78-2.88 (m, 2H) 3.41 (dt, J=9.76, 6.35 Hz, 1H) 3.47-3.51 (m, 1H) 3.78 (dt, J=9.64, 6.41 Hz, 1H) 3.85 (ddd, J=11.11, 7.69, 3.17 Hz, 1H) 4.55 (dd, J=4.64, 2.68 Hz, 1H) 7.20 (d, J=0.98 Hz, 1H) 7.32 (dd, J=7.81, 1.46 Hz, 1H) 7.74 (d, J=7.81 Hz, 1H).

### Example 469

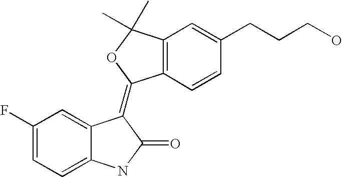

### Preparation of 5-fluoro-3-[5-(3-hydroxy-propyl)-3,3-dimethyl-3H-isobenzofuran-1-ylidene]-1,3-dihydro-indol-2-one

To a solution of 5-fluorooxindole (1.12 g, 7.4 mmol) in THF (25 ml) at 0° C. was added 1.0M LiHMDS/THF (14.9 ml) over several minutes. The solution was allowed to warm to room temperature, and then continuously stirred for 14 minutes. 3,3-dimethyl-5-[3-(tetrahydro-pyran-2-yloxy)-prop-1-ynyl]-3H-isobenzofuran-1-one (1.13 g, 3.72 mmol) in THF (2.0 ml) was added over 1 minute, and then the reaction mixture was rapidly stirred at room temperature for 3 hours. The reaction was quenched with 10% HCl aqueous solution (30 ml), concentrated HCl (2 ml) added, and the solution stirred at room temperature. After 1 hour the solution was heated to near reflux, then the heat removed, and the solution stirred for 1.5 hours at room temperature. The mixture was extracted with EtOAc, and the combined organic layers were washed with 4% HCl, brine, dried over anhydrous Na₂SO₄, and evaporated to an oil. The oil was then dissolved in THF (20 ml), MeOH (20 ml), and heated at 50° C. with Dowex 50WX8-200 resin (1.5 g) for 1.25 hours. The resin was removed by filtration and the solution evaporated. The oil was crystallized from EtOAc/hexane to give a yellow solid. The solid was triturated with MeOH to give the title compound as a yellow solid (827 mg, 63%).

¹H NMR (500 MHz, DMSO-d6) δ ppm 1.72 (s, 6H) 1.76-1.85 (m, 2H) 2.74-2.80 (m, 2H) 3.43-3.49 (m, 2H) 4.55 (t, J=5.13 Hz, 1H) 6.79 (dd, J=8.54, 4.64 Hz, 1H) 6.92 (ddd, J=9.76, 8.30, 2.44 Hz, 1H) 7.41 (dd, J=8.30, 1.46 Hz, 1H) 7.52 (d, J=0.98 Hz, 1H) 7.59 (dd, J=9.52, 2.68 Hz, 1H) 9.48 (d, J=8.30 Hz, 1H) 10.40 (s, 1H).

### Example 470

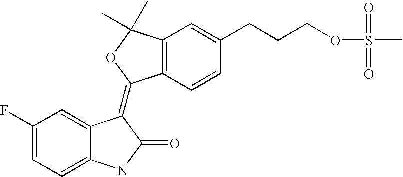

### Preparation of methanesulfonic acid 3-[1-(5-fluoro-2-oxo-1,2-dihydro-indol-3-ylidene)-3,3-dimethyl-1,3-dihydro-isobenzofuran-5-yl]-propyl ester

To a stirred solution of 5-fluoro-3-[5-(3-hydroxy-propyl)-3,3-dimethyl-3H-isobenzofuran-1-ylidene]-1,3-dihydro-indol-2-one (824 mg, 2.333 mmol) and triethylamine (0.72 ml, 5.13 mmol) in THF (17 ml) at room temperature was added methanesulfonyl chloride (0.217 ml, 2.80 mmol) over 1.5 minutes. The reaction was stirred for 15 minutes and then dissolved in EtOAc (150 ml). The EtOAc was washed with H₂O, diluted HCl, H₂O, and brine, dried over anhydrous Na₂SO₄, and evaporated to a yellow solid. The solid was triturated with 5% EtOAc in hexane to give the title compound as a yellow solid (967 mg, 96%).

¹H NMR (500 MHz, DMSO-d6) δ ppm 1.72 (s, 6H) 2.04-2.10 (m, 2H) 2.82-2.86 (m, 2H) 3.20 (s, 3H) 4.25 (t, J=6.35 Hz, 2H) 6.79 (dd, J=8.30, 4.88 Hz, 1H) 6.93 (ddd, J=9.76, 8.54, 2.69 Hz, 1H) 7.45 (dd, J=8.30, 1.46 Hz, 1H) 7.57 (d, J=0.98 Hz, 1H) 7.59 (dd, J=9.76, 2.44 Hz, 1H) 9.50 (d, J=7.81 Hz, 1H) 10.41 (s, 1H).

### Example 471

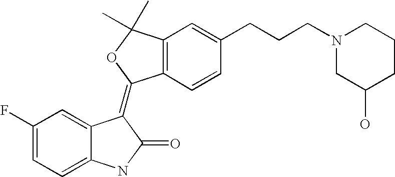

### Preparation of 5-fluoro-3-{5-[3-(3-hydroxy-piperidin-1-yl)-propyl]-3,3-dimethyl-3H-isobenzofuran-1-ylidene}-1,3-dihydro-indol-2-one

A solution of methanesulfonic acid 3-[1-(5-fluoro-2-oxo-1,2-dihydro-indol-3-ylidene)-3,3-dimethyl-1,3-dihydro-isobenzofuran-5-yl]-propyl ester (160 mg, 0.37 mmol) and 3-hydroxypiperidine (150 mg, 1.48 mmol) in DMF (1.2 ml) was heated at 80° C. for 40 minutes. The reaction was dissolved in EtOAc and washed with saturated aqueous NaHCO₃, H₂O, brine, dried over anhydrous Na₂SO₄, and evaporated to yellow oil. The oil was chromatographed eluting with gradient 4% to 8% methanol in CHCl₃ to give the title compound as a yellow foam (71 mg, 44%).

¹H NMR (500 MHz, CDCl₃) δ ppm 1.50-1.66 (m, 3H) 1.73 (s, 6H) 1.76-1.91 (m, 3H) 2.26 (s, 1H) 2.36-2.40 (m, 2H) 2.47 (br s, 3H) 2.75-2.79 (m, 2H) 3.84 (s, 1H) 6.75-6.78 (m, 1H) 6.82-6.87 (m, 1H) 7.14 (s, 1H) 7.35 (dd, J=8.30, 1.46 Hz, 1H) 7.69 (dd, J=9.76, 2.44 Hz, 1H) 7.82 (s, 1H) 9.55 (d, J=8.30 Hz, 1H).

### Example 472

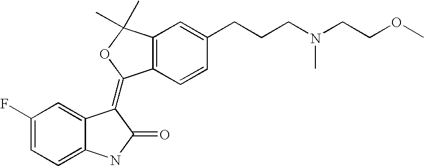

### 5-Fluoro-3-(5-{3-[(2-methoxy-ethyl)-methyl-amino]-propyl}-3,3-dimethyl-3H-isobenzofuran-1-ylidene)-1,3-dihydro-indol-2-one

Experimental procedure similar to 471.

¹H NMR (500 MHz, CDCl₃) δ ppm 1.73 (s, 6H) 1.87 (dt, J=15.13, 7.57 Hz, 2H) 2.29 (s, 3H) 2.42-2.46 (m, 2H) 2.58 (t, J=5.86 Hz, 2H) 2.75-2.79 (m, 2H) 3.36 (s, 3H) 3.48 (t, J=5.86 Hz, 2H) 6.75-6.78 (m, 1H) 6.84 (td, J=8.91, 2.69 Hz, 1H) 7.16 (s, 1H) 7.36 (dd, J=8.30, 1.46 Hz, 1H) 7.69 (dd, J=9.76, 2.44 Hz, 1H) 7.87 (broad s, 1H) 9.55 (d, J=8.30 Hz, 1H).

### Example 473

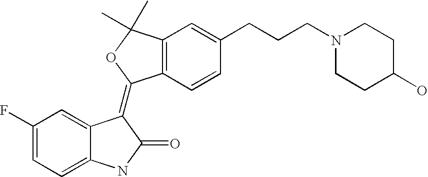

### 5-Fluoro-3-{5-[3-(4-hydroxy-piperidin-1-yl)-propyl]-3,3-dimethyl-3H-isobenzofuran-1-ylidene}-1,3-dihydro-indol-2-one

Procedure similar to Example 471.

¹H NMR (500 MHz, DMSO-d6) δ ppm 1.32-1.42 (m, 2H) 1.67-1.74 (m, 8H) 1.75-1.82 (m, 2H) 1.96 (t, J=10.01 Hz, 2H) 2.24-2.28 (m, 2H) 2.65-2.75 (m, 4H) 3.39-3.45 (m, 1H) 4.52 (d, J=4.39 Hz, 1H) 6.79 (dd, J=8.54, 4.64 Hz, 1H) 6.89-6.95 (m, 1H) 7.41 (dd, J=8.30, 1.46 Hz, 1H) 7.52 (d, J=0.98 Hz, 1H) 7.59 (dd, J=9.52, 2.69 Hz, 1H) 9.47 (d, J=7.81 Hz, 1H) 10.40 (s, 1H).

### Example 474

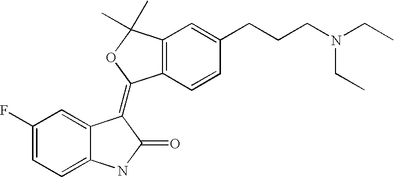

### 3-[5-(3-Diethylamino-propyl)-3,3-dimethyl-3H-isobenzofuran-1-ylidene]-5-fluoro-1,3-dihydro-indol-2-one

A solution of methanesulfonic acid 3-[1-(5-fluoro-2-oxo-1,2-dihydro-indol-3-ylidene)-3,3-dimethyl-1,3-dihydro-isobenzofuran-5-yl]-propyl ester (170 mg, 0.39 mmol) and diethylamine (0.30 ml, 2.92 mmol) in dioxane (1.6 ml) was heated at 75° C. in a pressure tube for 36 hours. The mixture was evaporated, dissolved in EtOAc, and the EtOAc washed with H₂O and brine. The aqueous layer was also extracted with CHCl₃. The organic layers were combined, dried over anhydrous Na₂SO₄, and then evaporated to a yellow film. The sample was passed through a plug of silica gel eluting with 10% methanol in CHCl₃ to give a yellow-orange solid. The solid was dissolved in EtOAc, washed with saturated aqueous NaHCO₃, brine, dried over anhydrous Na₂SO₄, and evaporated to a yellow solid. The solid was chromatographed eluting with CHCl₃/MeOH to give the title compound as a yellow solid (88 mg, 54%).

¹H NMR (500 MHz, DMSO-d6) δ ppm 0.93 (t, J=7.08 Hz, 6H) 1.72 (s, 6H) 1.77 (dt, J=14.77, 7.51 Hz, 2H) 2.41 (t, J=6.83 Hz, 2H) 2.47 (q, J=6.83 Hz, 4H) 2.71-2.76 (m, 2H) 6.79 (dd, J=8.30, 4.88 Hz, 1H) 6.92 (ddd, J=9.64, 8.42, 2.93 Hz, 1H) 7.42 (dd, J=8.30, 1.46 Hz, 1H) 7.53 (d, J=0.98 Hz, 1H) 7.59 (dd, J=9.52, 2.68 Hz, 1H) 9.48 (d, J=8.30 Hz, 1H) 10.40 (s, 1H).

### Example 475

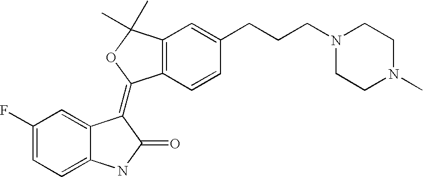

### 3-{3,3-Dimethyl-5-[3-(4-methyl-piperazin-1-yl)-propyl]-3H-isobenzofuran-1-ylidene}-5-fluoro-1,3-dihydro-indol-2-one

Experimental procedure similar to Example 483.

¹H NMR (500 MHz, CDCl₃) δ ppm 1.72 (s, 6H) 1.91 (br s, 2H) 2.38-2.79 (m, 15H) 6.74-6.77 (m, 1H) 6.81-6.86 (m, 1H) 7.14 (s, 1H) 7.33 (dd, J=8.06, 1.71 Hz, 1H) 7.68 (dd, J=9.52, 2.69 Hz, 1H) 7.72 (s, 1H) 9.54 (d, J=8.30 Hz, 1H).

### Example 476

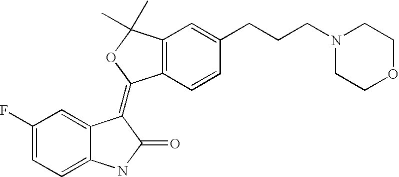

### 3-[3,3-Dimethyl-5-(3-morpholin-4-yl-propyl)-3H-isobenzofuran-1-ylidene]-5-fluoro-1,3-dihydro-indol-2-one

A solution of methanesulfonic acid 3-[1-(5-fluoro-2-oxo-1,2-dihydro-indol-3-ylidene)-3,3-dimethyl-1,3-dihydro-isobenzofuran-5-yl]-propyl ester (170 mg, 0.39 mmol) and morpholine (0.17 ml, 1.97 mmol) in THF (2.0 ml) was heated at 63° C. for 35 hours. The mixture was dissolved in EtOAc, and the EtOAc layer washed with H₂O/brine mixture, brine, dried over anhydrous Na₂SO₄, and evaporated to a yellow solid. The solid was recrystallized from EtOAc/hexane to give the title compound as a yellow solid (112 mg, 67%).

¹H NMR (500 MHz, DMSO-d6) δ ppm 1.70 (s, 6H) 1.76-1.83 (m, 2H) 2.26-2.30 (m, 2H) 2.33 (s, 4H) 2.71-2.75 (m, 2H) 3.54-3.58 (m, 4H) 6.77 (dd, J=8.30, 4.88 Hz, 1H) 6.89-6.93 (m, 1H) 7.41 (dd, J=8.30, 1.46 Hz, 1H) 7.52 (d, J=0.98 Hz, 1H) 7.57 (dd, J=9.76, 2.93 Hz, 1H) 9.46 (d, J=8.30 Hz, 1H) 10.39 (s, 1H).

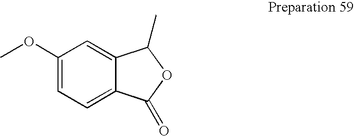

### Preparation of 5-methoxy-3-methyl-3H-isobenzofuran-1-one

Experimental procedure similar to Preparation 70.

¹H NMR (500 MHz, CDCl₃) δ ppm 1.63 (d, J=6.83 Hz, 3H) 3.92 (s, 3H) 5.49 (q, J=6.67 Hz, 1H) 6.86 (d, J=1.95 Hz, 1H) 7.04 (dd, J=8.54, 2.20 Hz, 1H) 7.81 (d, J=8.79 Hz, 1H).

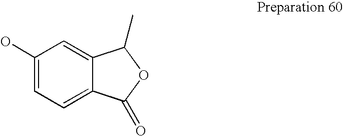

### Preparation of 5-hydroxy-3-methyl-3H-isobenzofuran-1-one

Experimental procedure similar to Preparation 55.

¹H NMR (500 MHz, DMSO-d6) δ ppm 1.49 (d, J=6.83 Hz, 3H) 5.52 (q, J=6.51 Hz, 1H) 6.91-6.94 (m, 2H) 7.62 (d, J=7.32 Hz, 1H) 10.66 (broad s, 1H).

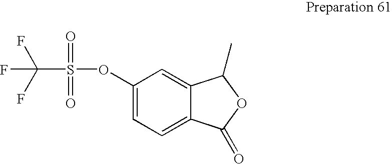

### Preparation of trifluoromethanesulfonic acid 3-methyl-1-oxo-1,3-dihydro-isobenzofuran-5-yl ester

Experimental procedure similar to Preparation 56.

¹H NMR (500 MHz, CDCl₃) δ ppm 1.67 (d, J=6.83 Hz, 3H) 5.59 (q, J=6.83 Hz, 1H) 7.37 (d, J=1.95 Hz, 1H) 7.43 (dd, J=8.54, 2.20 Hz, 1H) 7.99 (d, J=8.30 Hz, 1H).

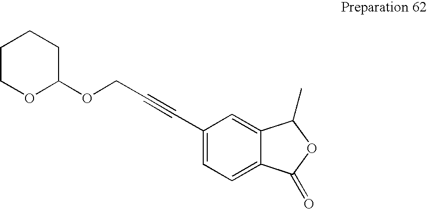

### Preparation of 3-Methyl-5-[3-(tetrahydro-pyran-2-yloxy)-prop-1-ynyl]-3H-isobenzofuran-1-one

Experimental procedure similar to Preparation 57.

¹H NMR (500 MHz, CDCl₃) δ ppm 1.52-1.70 (m, 7H) 1.73-1.89 (m, 2H) 3.54-3.59 (m, 1H) 3.85-3.90 (m, 1H) 4.44-4.56 (m, 2H) 4.88 (t, J=3.42 Hz, 1H) 5.52 (q, J=6.83 Hz, 1H) 7.49 (s, 1H) 7.56 (d, J=7.81 Hz, 1H) 7.81 (d, J=8.30 Hz, 1H).

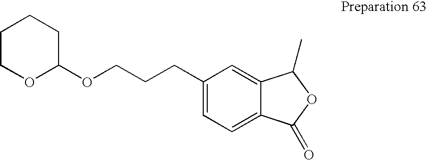

### Preparation of 3-methyl-5-[3-(tetrahydro-pyran-2-yloxy)-propyl]-3H-isobenzofuran-1-one

Experimental procedure similar to Example 58.

¹H NMR (500 MHz, CDCl₃) δ ppm 1.52-1.62 (m, 7H) 1.69-1.75 (m, 1H) 1.79-1.86 (m, 1H) 1.92-1.99 (m, 2H) 2.79-2.89 (m, 2H) 3.38-3.44 (m, J=9.64, 6.29, 6.29, 3.66 Hz, 1H) 3.47-3.52 (m, 1H) 3.75-3.81 (m, J=9.70, 6.50, 6.50, 2.68 Hz, 1H) 3.85 (ddd, J=10.98, 7.57, 2.93 Hz, 1H) 4.56 (dt, J=4.52, 2.38 Hz, 1H) 5.50 (q, J=6.83 Hz, 1H) 7.24 (s, 1H) 7.35 (d, J=7.81 Hz, 1H) 7.78 (d, J=7.81 Hz, 1H).

### Example 477

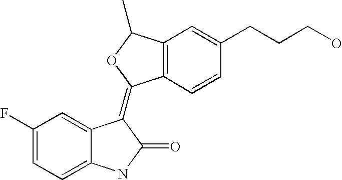

### Prepaaration of 5-Fluoro-3-[5-(3-hydroxy-propyl)-3-methyl-3H-isobenzofuran-1-ylidene]-1,3-dihydro-indol-2-one

Experimental procedure similar to Example 469.

¹H NMR (500 MHz, DMSO-d6) δ ppm 1.67 (d, J=6.83 Hz, 3H) 1.74-1.80 (m, 2H) 2.74-2.78 (m, 2H) 3.42-3.46 (m, 2H) 4.52-4.54 (m, 1H) 6.03 (q, J=6.35 Hz, 1H) 6.78 (dd, J=8.54, 4.64 Hz, 1H) 6.91 (ddd, J=9.64, 8.42, 2.93 Hz, 1H) 7.41 (dd, J=8.05, 1.22 Hz, 1H) 7.48 (s, 1H) 7.57 (dd, J=9.52, 2.68 Hz, 1H) 9.49 (d, J=8.30 Hz, 1H) 10.40 (s, 1H).

### Example 478

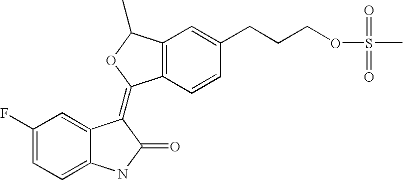

### Methanesulfonic acid 3-[1-(5-fluoro-2-oxo-1,2-dihydro-indol-3-ylidene)-3-methyl-1,3-dihydro-isobenzofuran-5-yl]-propyl ester

Experimental procedure similar to Example 470.

¹H NMR (500 MHz, DMSO-d6) δ ppm 1.67 (d, J=6.83 Hz, 3H) 2.05 (dq, J=9.03, 6.43 Hz, 2H) 2.81-2.85 (m, 2H) 3.18 (s, 3H) 4.23 (t, J=6.10 Hz, 2H) 6.04 (q, J=6.83 Hz, 1H) 6.78 (dd, J=8.54, 4.64 Hz, 1H) 6.92 (ddd, J=9.52, 8.54, 2.93 Hz, 1H) 7.45 (d, J=8.30 Hz, 1H) 7.52 (s, 1H) 7.57 (dd, J=9.52, 2.69 Hz, 1H) 9.51 (d, J=8.30 Hz, 1H) 10.41 (s, 1H).

### Example 479

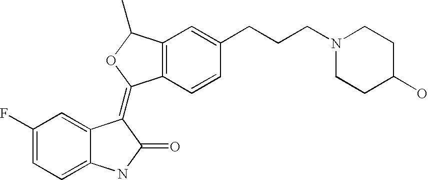

### 5-Fluoro-3-{5-[3-(4-hydroxy-piperidin-1-yl)-propyl]-3-methyl-3H-isobenzofuran-1-ylidene}-1,3-dihydro-indol-2-one

Experimental procedure similar to Example 471.

¹H NMR (500 MHz, DMSO-d6) δ ppm 1.33-1.41 (m, 2H) 1.66-1.73 (m, 5H) 1.74-1.83 (m, 2H) 1.96 (s, 2H) 2.26 (t, J=6.83 Hz, 2H) 2.65-2.75 (m, 4H) 3.39-3.46 (m, 1H) 4.52 (d, J=4.39 Hz, 1H) 6.04 (q, J=6.67 Hz, 1H) 6.79 (dd, J=8.30, 4.88 Hz, 1H) 6.90-6.95 (m, 1H) 7.43 (dd, J=8.30, 0.98 Hz, 1H) 7.50 (s, 1H) 7.58 (dd, J=9.76, 2.93 Hz, 1H) 9.49 (d, J=8.30 Hz, 1H) 10.41 (s, 1H).

### Example 480

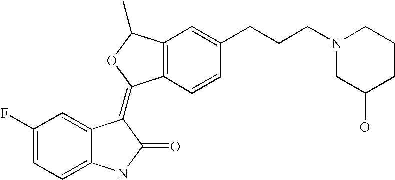

### 5-Fluoro-3-{5-[3-(3-hydroxy-piperidin-1-yl)-propyl]-3-methyl-3H-isobenzofuran-1-ylidene}-1,3-dihydro-indol-2-one

Experimental procedure similar to Example 471.

¹H NMR (500 MHz, CDCl₃) δ ppm 1.50-1.64 (m, 3H) 1.73 (d, J=6.83 Hz, 3H) 1.76-1.91 (m, 3H) 2.26 (s, 1H) 2.36-2.40 (m, 2H) 2.47 (br s, 3H) 2.77 (t, J=7.81 Hz, 2H) 3.83 (s, 1H) 5.88 (q, J=6.51 Hz, 1H) 6.76 (dd, J=8.54, 4.64 Hz, 1H) 6.82-6.87 (m, 1H) 7.20 (s, 1H) 7.36 (dd, J=8.30, 0.98 Hz, 1H) 7.70 (dd, J=9.52, 2.68 Hz, 1H) 7.72 (s, 1H) 9.57 (d, J=8.30 Hz, 1H).

### Example 481

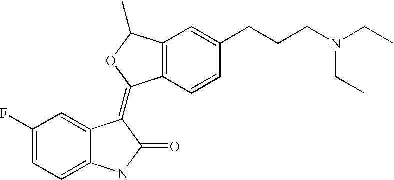

### 3-[5-(3-Diethylamino-propyl)-3-methyl-3H-isobenzofuran-1-ylidene]-5-fluoro-1,3-dihydro-indol-2-one

Experimental procedure similar to Example 474.

¹H NMR (500 MHz, CDCl₃) δ ppm 1.04 (t, J=7.08 Hz, 6H) 1.73 (d, J=6.83 Hz, 3H) 1.87 (dt, J=14.16, 7.08 Hz, 2H) 2.51 (t, J=7.32 Hz, 2H) 2.54-2.61 (m, 4H) 2.77 (t, J=7.81 Hz, 2H) 5.88 (q, J=6.83 Hz, 1H) 6.76 (dd, J=8.79, 4.39 Hz, 1H) 6.84 (td, J=8.91, 2.69 Hz, 1H) 7.22 (s, 1H) 7.38 (d, J=8.30 Hz, 1H) 7.70 (dd, J=9.52, 2.68 Hz, 1H) 7.73 (s, 1H) 9.58 (d, J=8.30 Hz, 1H).

### Example 482

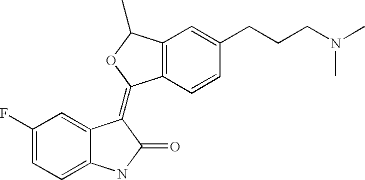

### 3-[5-(3-Dimethylamino-propyl)-3-methyl-3H-isobenzofuran-1-ylidene]-5-fluoro-1,3-dihydro-indol-2-one

A solution of methanesulfonic acid 3-[1-(5-fluoro-2-oxo-1,2-dihydro-indol-3-ylidene)-3-methyl-1,3-dihydro-isobenzofuran-5-yl]-propyl ester (292 mg, 0.70 mmol) and 2.0M dimethylamine/THF solution (2.0 ml) in THF (2.0 ml) was heated at 70° C. in a pressure tube for 20 hours. The mixture was dissolved in EtOAc, and the EtOAc layer washed with H₂O/brine mixture, dried over anhydrous Na₂SO₄, and evaporated to a yellow-orange solid. The solid was recrystallized from EtOAc/hexane to give the title compound as a yellow solid (172 mg, 67%).

¹H NMR (500 MHz, DMSO-d6) δ ppm 1.67 (d, J=6.83 Hz, 3H) 1.71-1.78 (m, 2H) 2.12 (s, 6H) 2.21 (t, J=7.08 Hz, 2H) 2.72 (t, J=7.81 Hz, 2H) 6.03 (q, J=6.83 Hz, 1H) 6.78 (dd, J=8.30, 4.39 Hz, 1H) 6.89-6.94 (m, 1H) 7.42 (dd, J=8.06, 1.22 Hz, 1H) 7.48 (s, 1H) 7.57 (dd, J=9.76, 2.93 Hz, 1H) 9.49 (d, J=8.30 Hz, 1H) 10.40 (s, 1H).

### Example 483

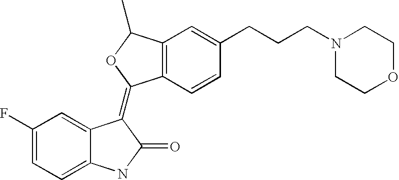

### 5-Fluoro-3-[3-methyl-5-(3-morpholin-4-yl-propyl)-3H-isobenzofuran-1-ylidene]-1,3-dihydro-indol-2-one

A solution of methanesulfonic acid 3-[1-(5-fluoro-2-oxo-1,2-dihydro-indol-3-ylidene)-3-methyl-1,3-dihydro-isobenzofuran-5-yl]-propyl ester (292 mg, 0.70 mmol) and morpholine (0.31 ml, 3.5 mmol) in dioxane (3.0 ml) was heated at 85° C. for 17 hours. The mixture was dissolved in EtOAc, and the EtOAc layer washed with H₂O/brine mixture, dried over anhydrous Na₂SO₄, and evaporated to a yellow solid. The solid was recrystallized from EtOAc/hexane to give the title compound as a yellow solid (241 mg, 84%).

¹H NMR (500 MHz, DMSO-d6) δ ppm 1.67 (d, J=6.35 Hz, 3H) 1.74-1.83 (m, 2H) 2.28 (t, J=7.32 Hz, 2H) 2.33 (s, 4H) 2.74 (t, J=7.81 Hz, 2H) 3.56 (t, J=4.64 Hz, 4H) 6.03 (q, J=6.35 Hz, 1H) 6.78 (dd, J=8.30, 4.39 Hz, 1H) 6.89-6.94 (m, 1H) 7.42 (dd, J=8.30, 0.98 Hz, 1H) 7.49 (s, 1H) 7.57 (dd, J=9.76, 2.44 Hz, 1H) 9.49 (d, J=8.30 Hz, 1H) 10.40 (s, 1H).

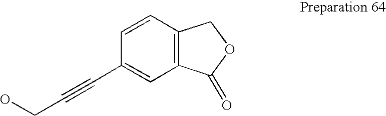

### Preparation of 6-(3-hydroxy-prop-1-ynyl)-3H-isobenzofuran-1-one

A solution of 6-bromophthalide (533 mg, 2.5 mmol), propargyl alcohol (0.59 ml, 10.0 mmol), and triethylamine (0.70 ml, 5.0 mmol) in DMF (7.0 ml) at room temperature was degassed with argon. Then copper(I) iodide (143 mg, 0.75 mmol) and tetrakis(triphenylphosphine)palladium (217 mg, 0.19 mmol) were added and the reaction was stirred at 70° C. for 1.5 hours. The reaction was added to methanol (50 ml) and H₂O (5 ml) and the dark precipitate was removed by filtration. The solution was evaporated, dissolved in EtOAc, and washed with 2% HCl, H₂O, brine, dried with anhydrous Na₂SO₄, and evaporated to brown oil. The oil was chromatographed eluting with gradient 40% to 50% EtOAc in hexane to give the title compound as a yellow solid (147 mg, 31%).

¹H NMR (500 MHz, CDCl₃) δ ppm 1.81 (t, J=6.04 Hz, 1H) 4.53 (d, J=5.49 Hz, 2H) 5.33 (s, 2H) 7.46 (d, J=8.05 Hz, 1H) 7.73 (d, J=8.05 Hz, 1H) 7.97 (s, 1H).

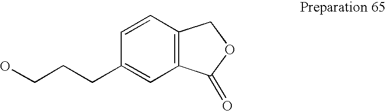

### Preparation of 6-(3-hydroxy-propyl)-3H-isobenzofuran-1-one

A mixture of 6-(3-hydroxy-prop-1-ynyl)-3H-isobenzofuran-1-one (142 mg, 0.76 mmol) and 10% palladium on carbon (25 mg) in MeOH (25 ml) was shaken under 50 psi of hydrogen for 18.5 hours. The catalyst was removed by filtration through celite and rinsed with MeOH. The combined filtrates were evaporated to oil. The oil was chromatographed eluting CHCl₃ and then 50% EtOAc in hexane to give the title compound as a white wax-like solid (142 mg, 98%).

¹H NMR (500 MHz, CDCl₃) δ ppm 1.37 (t, J=4.03 Hz, 1H) 1.90-1.96 (m, 2H) 2.83-2.87 (m, 2H) 3.67-3.72 (m, 2H) 5.30 (s, 2H) 7.41 (d, J=8.06 Hz, 1H) 7.54 (d, J=7.69 Hz, 1H) 7.76 (s, 1H).

### Example 484

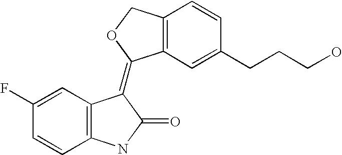

### 5-Fluoro-3-[6-(3-hydroxy-propyl)-3H-isobenzofuran-1-ylidene]-1,3-dihydro-indol-2-one

To a solution of 5-fluorooxindole (216 mg, 1.43 mmol) in THF (5.5 ml) at 0° C. was added 1.0M LiHMDS/THF (3.6 ml) over 2 minutes. After the ice bath was removed, the solution was stirred for 10 minutes at room temperature, and then 6-(3-hydroxy-propyl)-3H-isobenzofuran-1-one (137 mg, 0.71 mmol) in THF (1.5 ml) was added. The reaction mixture was rapidly stirred at room temperature for 2.5 hours. The reaction was quenched into 5% HCl aqueous solution (50 ml), and the solution was warmed until the precipitate had dissolved. H₂O (20 ml) was added, and the solution was allowed to cool to room temperature giving a yellow-orange precipitate. The precipitate was filtered and rinsed with H₂O. The solid was dissolved in EtOAc/MeOH, washed with H₂O, brine, dried with anhydrous Na₂SO₄, and evaporated to a solid. The solid was triturated with EtOAc to give the title compound as a yellow-orange solid (110 mg, 48%).

¹H NMR (500 MHz, DMSO-d6) δ ppm 1.79 (dt, J=15.01, 6.65 Hz, 2H) 2.73-2.77 (m, 2H) 3.44-3.48 (m, 2H) 4.53 (td, J=5.13, 0.98 Hz, 1H) 5.79 (s, 2H) 6.80 (dd, J=8.30, 4.88 Hz, 1H) 6.91-6.96 (m, 1H) 7.52-7.55 (m, 1H) 7.57-7.61 (m, 2H) 9.53 (s, 1H) 10.40 (s, 1H).

### Example 485

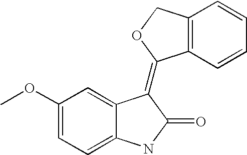

### 3-(3H-Isobenzofuran-1-ylidene)-5-methoxy-1,3-dihydro-indol-2-one

Experimental procedure similar to Example 426.

¹H NMR (500 MHz, DMSO-d6) δ ppm 3.75 (s, 3H) 5.82 (s, 2H) 6.73 (s, 2H) 7.47 (s, 1H) 7.53-7.60 (m, 1H) 7.66-7.67 (m, 2H) 9.68 (d, J=8.30 Hz, 1H) 10.25 (s, 1H)

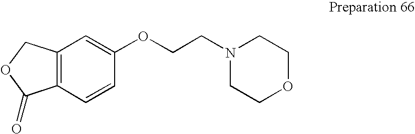

### Preparation of 5-(2-morpholin-4-yl-ethoxy)-3H-isobenzofuran-1-one

A mixture of 5-hydroxyphthalide (2.0 g, 13.3 mmol), 4-(2-chloroethyl)morpholine hydrochloride (3.72 g, 20 mmol) and potassium carbonate (5.5 g, 40 mmol) in DMF (10 ml) was stirred at 60° C. for 16 hours. The reaction was cooled to room temperature, and then partitioned between water (100 ml) and EtOAc (75 ml). The aqueous layer was extracted with EtOAc (4×75 ml). The combined organic layers were washed with 1M NaOH solution (50 ml), brine (100 ml), and then dried over anhydrous Na₂SO₄ and concentrated. The title compound was obtained as off-white solid (2.5 g, 71%).

¹H NMR (500 MHz, CDCl₃) δ ppm 2.72 (br s, 4H) 2.96 (br s, 2H) 3.82 (br s, 4H) 4.30 (br s, 2H) 5.25 (s, 2H) 6.95 (s, 1H) 7.05 (dd, J=8.30, 1.95 Hz, 1H) 7.83 (d, J=8.30 Hz, 1H)

### Example 486

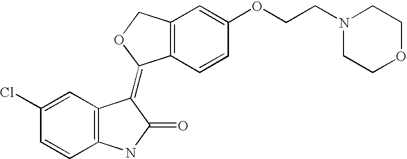

### 5-Chloro-3-[5-(2-morpholin-4-yl-ethoxy)-3H-isobenzofuran-1-ylidene]-1,3-dihydro-indol-2-one

To a stirred solution of 5-chlorooxindole (382 mg, 2.28 mmol) in anhydrous THF (20 ml) under nitrogen was added 1.0M LiHMDS/THF solution (4.8 ml, 4.8 mmol). The mixture was stirred at room temperature for 10 minutes, and 5-(2-Morpholin-4-yl-ethoxy)-3H-isobenzofuran-1-one (300 mg, 1.14 mmol) was added. After stirring at room temperature for 3 hours, the mixture was poured into 1M HCl aqueous solution (20 ml) and heated at 60° C. for 30 minutes. The mixture was cooled to room temperature, and then poured into water (200 ml) with vigorous stirring. 5M NaOH was added until the pH was about 9. The resulting solid was separated, rinsed with water, and dried to give the title compound as a yellow solid (405 mg, 86%).

¹H NMR (500 MHz, DMSO-d6) δ ppm 2.48 (br s, 4H) 2.73 (t, J=5.61 Hz, 2H) 3.58 (t, J=4.39 Hz, 4H) 4.22 (t, J=5.86 Hz, 2H) 5.79 (s, 2H) 6.81 (d, J=8.30 Hz, 1H) 7.11 (dd, J=8.06, 2.20 Hz, 1H) 7.14 (dd, J=8.79, 2.44 Hz, 1H) 7.26 (s, 1H) 7.76 (d, J=1.95 Hz, 1H) 9.54 (d, J=8.79 Hz, 1H) 10.50 (s, 1H)

The following Examples 487-495 were prepared using the experiment procedure described in Example 486, but with the appropriate reagent, reaction conditions and reactant substitutions that will be readily realized by those of ordinary skill in this art without the exercise of undue experimentation

### Example 487

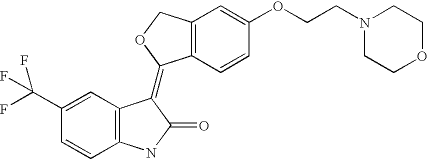

### 3-[5-(2-Morpholin-4-yl-ethoxy)-3H-isobenzofuran-1-ylidene]-5-trifluoromethyl-1,3-dihydro-indol-2-one

¹H NMR (500 MHz, DMSO-d6) δ ppm 2.48 (br s, 4H) 2.74 (t, J=5.61 Hz, 2H) 3.58 (t, J=4.39 Hz, 4H) 4.24 (t, J=5.61 Hz, 2H) 5.84 (s, 2H) 6.98 (d, J=8.30 Hz, 1H) 7.17 (dd, J=9.03, 2.20 Hz, 1H) 7.28 (s, 1H) 7.44 (d, J=8.30 Hz, 1H) 8.04 (s, 1H) 9.56 (d, J=8.79 Hz, 1H) 10.79 (s, 1H)

### Example 488

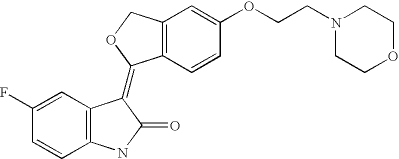

### 5-Fluoro-3-[5-(2-morpholin-4-yl-ethoxy)-3H-isobenzofuran-1-ylidene]-1,3-dihydro-indol-2-one

¹H NMR (500 MHz, DMSO-d6) δ ppm 2.48 (br s, 4H) 2.74 (t, J=5.61 Hz, 2H) 3.58 (t, J=4.39 Hz, 4H) 4.23 (t, J=5.61 Hz, 2H) 5.77 (s, 2H) 6.77 (dd, J=8.54, 4.64 Hz, 1H) 6.85-6.94 (m, 1H) 7.15 (dd, J=8.79, 2.44 Hz, 1H) 7.25 (s, 1H) 7.54 (dd, J=9.76, 2.44 Hz, 1H) 9.56 (d, J=8.79 Hz, 1H) 10.39 (s, 1H)

### Example 489

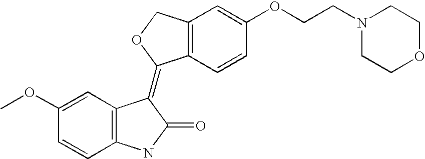

### 5-Methoxy-3-[5-(2-morpholin-4-yl-ethoxy)-3H-isobenzofuran-1-ylidene]-1,3-dihydro-indol-2-one

¹H NMR (500 MHz, DMSO-d6) δ ppm 2.49 (br s, 4H) 2.74 (t, J=5.61 Hz, 2H) 3.58 (t, J=4.88 Hz, 4H) 3.73 (s, 3H) 4.22 (t, J=5.61 Hz, 2H) 5.75 (s, 2H) 6.65-6.73 (m, 2H) 7.13 (dd, J=9.03, 2.20 Hz, 1H) 7.23 (s, 1H) 7.42 (d, J=2.44 Hz, 1H) 9.58 (d, J=9.28 Hz, 1H) 10.17 (s, 1H)

### Example 490

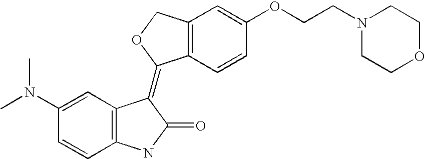

### 5-Dimethylamino-3-[5-(2-morpholin-4-yl-ethoxy)-3H-isobenzofuran-1-ylidene]-1,3-dihydro-indol-2-one

¹H NMR (500 MHz, DMSO-d6) δ ppm 2.48 (br s, 4H) 2.73 (t, J=5.61 Hz, 2H) 2.83 (s, 6H) 3.58 (t, J=4.39 Hz, 4H) 4.22 (t, J=5.61 Hz, 2H) 5.74 (s, 2H) 6.56 (dd, J=8.79, 2.44 Hz, 1H) 6.66 (d, J=8.30 Hz, 1H) 7.11 (dd, J=8.79, 1.95 Hz, 1H) 7.22 (s, 1H) 7.39 (d, J=2.44 Hz, 1H) 9.59 (d, J=8.79 Hz, 1H) 10.04 (s, 1H)

### Example 491

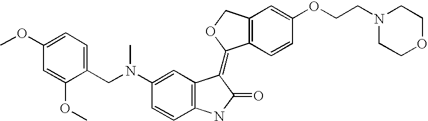

### 5-[(2,4-Dimethoxy-benzyl)-methyl-amino]-3-[5-(2-morpholin-4-yl-ethoxy)-3H-isobenzofuran-1-ylidene]-1,3-dihydro-indol-2-one

¹H NMR (500 MHz, DMSO-d6) δ ppm 2.48 (br s, 4H) 2.73 (t, J=5.61 Hz, 2H) 2.89 (s, 3H) 3.58 (t, J=4.88 Hz, 4H) 3.72 (s, 3H) 3.83 (s, 3H) 4.21 (t, J=5.86 Hz, 2H) 4.31 (s, 2H) 5.71 (s, 2H) 6.43 (dd, J=8.30, 2.44 Hz, 1H) 6.48 (dd, J=8.30, 2.44 Hz, 1H) 6.57 (d, J=2.44 Hz, 1H) 6.62 (d, J=8.79 Hz, 1H) 6.98 (d, J=8.30 Hz, 1H) 7.11 (dd, J=9.03, 2.20 Hz, 1H) 7.20 (d, J=1.95 Hz, 1H) 7.40 (d, J=2.44 Hz, 1H) 9.58 (d, J=8.79 Hz, 1H) 9.99 (s, 1H)

### Example 492

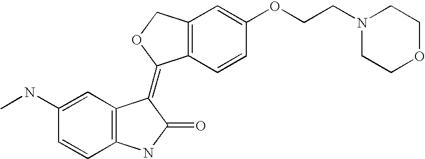

### 5-Methylamino-3-[5-(2-morpholin-4-yl-ethoxy)-3H-isobenzofuran-1-ylidene]-1,3-dihydro-indol-2-one

¹H NMR (500 MHz, DMSO-d6) δ ppm 2.54 (br s, 4H) 2.66 (s, 3H) 2.79 (br s, 2H) 3.08 (br s, 1H) 3.60 (br s, 4H) 4.23 (t, J=5.37 Hz, 2H) 5.72 (s, 2H) 6.31 (dd, J=8.06, 2.20 Hz, 1H) 6.58 (d, J=8.30 Hz, 1H) 7.11 (dd, J=8.79, 2.44 Hz, 1H) 7.19 (dd, J=9.28, 1.95 Hz, 2H) 9.60 (d, J=8.79 Hz, 1H) 9.94 (s, 1H)

### Example 493

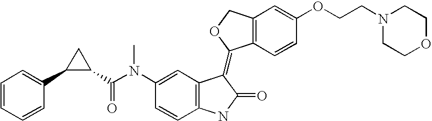

### 2-Phenyl-cyclopropanecarboxylic acid methyl-{3-[5-(2-morpholin-4-yl-ethoxy)-3H-isobenzofuran-1-ylidene]-2-oxo-2,3-dihydro-1H-indol-5-yl}-amide

MS (ES+): 552.2495 (MH⁺)

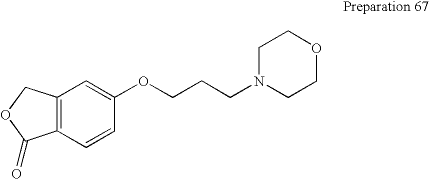

### Preparation of 5-(3-morpholin-4-yl-propoxy)-3H-isobenzofuran-1-one

A mixture of 5-hydroxyphthalide (500 mg, 3.3 mmol), 4-(3-Chloro-propyl)-morpholine (654 mg, 4.0 mmol) and potassium carbonate (553 mg, 4.0 mmol) in DMF (7 ml) was stirred at 60° C. for 16 hours. The reaction was cooled to room temperature, and partitioned between water (75 ml) and EtOAc (50 ml). The aqueous layer was extracted with EtOAc (2×50 ml). The combined organic layers were washed with water (100 ml), brine (60 ml), and dried over anhydrous Na₂SO₄. Removal of the solvent gave the title compound as white crystals (844 mg, 92%).

¹H NMR (500 MHz, CDCl₃) δ ppm 2.16 (br s, 2H) 2.67 (br s, 6H) 3.85 (br s, 4H) 4.16 (t, J=6.35 Hz, 2H) 5.26 (s, 2H) 6.93 (s, 1H) 7.04 (dd, J=8.54, 2.20 Hz, 1H) 7.83 (d, J=8.79 Hz, 1H)

### Example 494

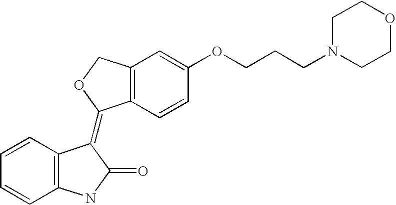

### 3-[5-(3-Morpholin-4-yl-propoxy)-3H-isobenzofuran-1-ylidene]-1,3-dihydro-indol-2-one

¹H NMR (500 MHz, DMSO-d6) δ ppm 1.89-1.96 (m, 2H) 2.37 (br s, 4H) 2.44 (t, J=7.32 Hz, 2H) 3.57 (t, J=4.64 Hz, 4H) 4.14 (t, J=6.35 Hz, 2H) 5.75 (s, 2H) 6.81 (d, J=7.32 Hz, 1H) 6.94 (t, J=7.57 Hz, 1H) 7.07 (t, J=7.57 Hz, 1H) 7.11 (dd, J=9.03, 2.20 Hz, 1H) 7.21 (s, 1H) 7.79 (d, J=7.81 Hz, 1H) 9.56 (d, J=8.79 Hz, 1H) 10.35 (s, 1H)

### Example 495

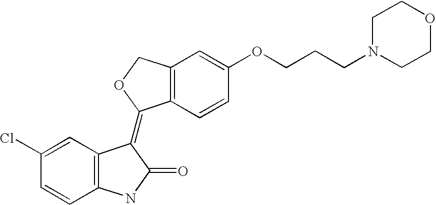

### 5-Chloro-3-[5-(3-morpholin-4-yl-propoxy)-3H-isobenzofuran-1-ylidene]-1,3-dihydro-indol-2-one

¹H NMR (500 MHz, DMSO-d6) δ ppm 1.89-1.96 (m, 2H) 2.37 (br s, 4H) 2.44 (t, J=7.08 Hz, 2H) 3.57 (t, J=4.39 Hz, 4H) 4.15 (t, J=6.35 Hz, 2H) 5.79 (s, 2H) 6.81 (d, J=8.30 Hz, 1H) 7.09-7.15 (m, 2H) 7.25 (d, J=2.44 Hz, 1H) 7.76 (d, J=2.44 Hz, 1H) 9.54 (d, J=9.28 Hz, 1H) 10.50 (s, 1H)

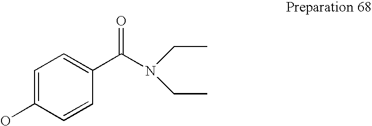

### Preparation of N,N-diethyl-4-hydroxy-benzamide

To a solution containing 4-hydroxybenzoic acid (5.11 g, 37.0 mmol), diethylamine (8.42 ml, 81.4 mmol) and 1-hydroxybenzotriazole hydrate (5.25 g, 38.9 mmol) in DMF (50 ml) at room temperature was added 1-[3-(dimethylamino)propyl]-3-ethylcarbodiimide hydrochloride (7.80 g, 40.7 mmol) in portions over 15 minutes. After 22 hours the reaction was poured into 5% HCl and extracted with EtOAc. The EtOAc layer was washed with H₂O, saturated NaHCO₃, and brine and then dried over anhydrous Na₂SO₄. Concentration of the EtOAc solution on the rotary evaporator gave a precipitate which was filtered and rinsed with 30% EtOAc in hexane to give the title compound as a white solid (4.22 g, 59%).

¹H NMR (500 MHz, DMSO-d6) δ ppm 1.08 (t, J=6.83 Hz, 6H) 3.30 (s, 4H) 6.76-6.79 (m, 2H) 7.17-7.20 (m, 2H) 9.74 (s, 1H).

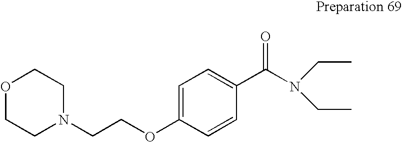

### Preparation of N,N-diethyl-4-(2-morpholin-4-yl-ethoxy)-benzamide

To a solution of N,N-diethyl-4-hydroxy-benzamide (4.84 g, 25.0 mmol) in DMF (60 ml) at room temperature was added K₂CO₃ (13.0 g, 93.8 mmol) and 4-(2-chloroethyl)morpholine hydrochloride (4.42 g, 23.8 mmol). Upon stirring for 5 minutes the mixture was heated at 60° C. for 12 hours. The reaction was filtered and the solids were rinsed with EtOAc. The EtOAc filtrate was evaporated to light yellow oil. The oil was dissolved in EtOAc and washed with H₂O, 0.5M NaOH, H₂O, brine, dried with anhydrous Na₂SO₄, and evaporated to give the title compound as a light yellow oil (6.64 g, 91%).

¹H NMR (500 MHz, CDCl₃) δ ppm 1.18 (br s, 6H) 2.57-2.60 (m, 4H) 2.81 (t, J=5.61 Hz, 2H) 3.41 (br d, 4H) 3.73-3.75 (m, 4H) 4.13 (t, J=5.61 Hz, 2H) 6.90 (d, J=8.30 Hz, 2H) 7.33 (d, J=8.30 Hz, 2H).

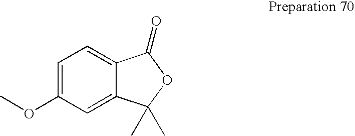

### Preparation of 5-methoxy-3,3-dimethyl-3H-isobenzofuran-1-one

To a solution of 4-methoxybenzoic acid (4.169 g, 27.4 mmol) in THF (200 ml) at room temperature was added. N,N,N′,N′-tetramethylethylenediamine (8.68 ml, 57.5 mmol). The solution was cooled to −78° C. and then added 1.4M s-BuLi/cyclohexane solution (47 ml, 65.8 mmol) by adding first 21 ml s-BuLi over 5 min, stopping addition for 5 minutes, then adding the remaining solution over 15 minutes. After stirring at −78° C. for 40 minutes, acetone (5.0 ml, 68.0 mmol) was added drop wise over 4 minutes. Upon stirring 10 minutes, the cold bath is removed and the reaction was allowed to warm to room temperature, and then 10% HCl (100 ml) was added. After rapidly stirred at room temperature for 2 hours, the mixture was extracted with EtOAc and the combined organic layers washed with saturated NaHCO₃, H₂O, and brine, dried with anhydrous Na₂SO₄ and evaporated. The clear oil was crystallized from EtOAc/hexane to give a white solid (1.679 g). The filtrate was chromatographed by eluting with 10% to 40% EtOAc in hexane to give an additional solid (0.524 g). The combined lots gave the title compound as a white solid (2.203 g, 42%).

¹H NMR (500 MHz, CDCl₃) δ ppm 1.64 (s, 6H) 3.91 (s, 3H) 6.80 (d, J=1.95 Hz, 1H) 6.99-7.01 (m, 1H) 7.77 (d, J=8.30 Hz, 1H).

### Example 496

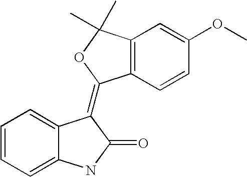

### 3-(5-Methoxy-3,3-dimethyl-3H-isobenzofuran-1-ylidene)-1,3-dihydro-indol-2-one

To a solution of oxindole (104 mg, 0.78 mmol) in dimethoxyethane (2.0 ml) cooled to 0□C was added 1.0M LiHMDS/THF (1.6 ml) over 3 minutes. The mixture was stirred for 3 minutes at 0° C., and then 7 minutes after the ice bath removed. The solid 5-methoxy-3,3-dimethyl-3H-isobenzofuran-1-one (134.6 mg, 0.70 mmol) was added in one portion and the reaction mixture rapidly stirred at room temperature for 22 hours. The reaction was quenched into 5% HCl aqueous solution (50 ml) to give a yellow precipitate. The solid was filtered and rinsed with H₂O and 30% EtOAc in hexane. The solid was dissolved in EtOAc and washed with H₂O, brine, dried over anhydrous Na₂SO₄, and evaporated. The resulting solid was recrystallized from EtOAc/hexane to give 3-(5-methoxy-3,3-dimethyl-3H-isobenzofuran-1-ylidene)-1,3-dihydro-indol-2-one as a yellow solid (33.5 mg, 16%).

¹H NMR (500 MHz, DMSO-d6) δ ppm 1.71 (s, 6H) 3.90 (s, 3H) 6.81 (d, J=7.32 Hz, 1H) 6.95 (td, J=7.57, 0.98 Hz, 1H) 7.08 (td, J=7.57, 0.98 Hz, 1H) 7.11 (dd, J=8.79, 2.44 Hz, 1H) 7.27 (d, J=1.95 Hz, 1H) 7.82 (d, J=7.81 Hz, 1H) 9.53 (d, J=8.79 Hz, 1H) 10.33 (s, 1H).

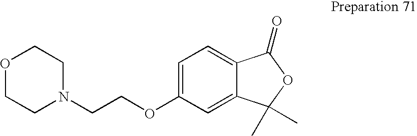

### Preparation of 3,3-dimethyl-5-(2-morpholin-4-yl-ethoxy)-3H-isobenzofuran-1-one

To a solution of N,N-diethyl-4-(2-morpholin-4-yl-ethoxy)-benzamide (408 mg, 1.33 mmol) and N,N,N′,N′-tetramethylethylenediamine (0.211 ml, 1.4 mmol) in THF (9.0 ml) at −78° C. was added 1.4M s-BuLi/cyclohexane solution (1.0 ml, 1.4 mmol) over 5 minutes. Upon stirring for 1 hour, acetone (0.488 ml, 6.65 mmol) was added and after 5 minutes the cold bath removed allowing the reaction to warm to room temperature. After 5 minutes at room temperature, 4% HCl aqueous solution was added until pH 2. The mixture was then basified by adding saturated NaHCO₃ solution. The mixture was extracted with EtOAc, and the combined organic layers were washed with H₂O, brine, dried over anhydrous Na₂SO₄, and evaporated to light yellow oil. The oil was dissolved in toluene (12 ml), catalytic p-TsOH added, and the mixture heated at reflux for 3 hours. The solution was decanted from the residue, evaporated, and chromatographed by eluting with gradient 1% to 3% MeOH in CHCl₃ to give the title compound as an off-white solid (150 mg, 39%).

¹H NMR (500 MHz, CDCl₃) δ ppm 1.63 (s, 6H) 2.58-2.61 (m, 4H) 2.85 (t, J=5.61 Hz, 2H) 3.73-3.76 (m, 4H) 4.20 (t, J=5.61 Hz, 2H) 6.82 (d, J=2.44 Hz, 1H) 7.01 (dd, J=8.30, 1.95 Hz, 1H) 7.77 (d, J=8.30 Hz, 1H).

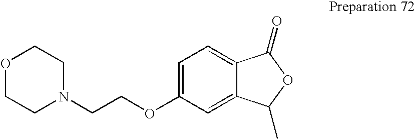

### Preparation of 3-methyl-5-(2-morpholin-4-yl-ethoxy)-3H-isobenzofuran-1-one

Experimental procedure similar to Preparation 71.

¹H NMR (500 MHz, CDCl₃) δ ppm 1.61 (d, J=6.83 Hz, 3H) 2.58-2.61 (m, 4H) 2.85 (t, J=5.61 Hz, 2H) 3.73-3.75 (m, 4H) 4.18-4.21 (m, 2H) 5.47 (q, J=6.67 Hz, 1H) 6.86 (d, J=1.95 Hz, 1H) 7.03 (dd, J=8.54, 2.20 Hz, 1H) 7.79 (d, J=8.30 Hz, 1H).

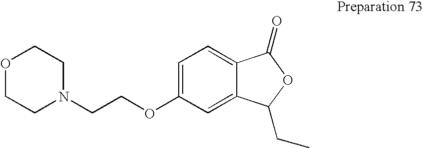

### 3-Ethyl-5-(2-morpholin-4-yl-ethoxy)-3H-isobenzofuran-1-one

To a solution of N,N-diethyl-4-(2-morpholin-4-yl-ethoxy)-benzamide (408 mg, 1.33 mmol) and N,N,N′,N′-tetramethylethylenediamine (0.211 ml, 1.4 mmol) in THF (9.0 ml) at −78° C. was added 1.4M s-BuLi/cyclohexane solution (1.0 ml, 1.4 mmol) over 5 minutes. Upon stirring for 1 hour, propionaldehyde (0.48 ml, 6.65 mmol) was added and after 5 minutes the cold bath removed allowing the reaction to warm to room temperature. After 5 minutes at room temperature, 10% HCl aqueous solution (8 ml) was added and the mixture stirred for 1 hour. The solution was then basified by adding saturated NaHCO₃ solution, extracted with EtOAc. The combined organic layers were washed with H₂O, brine, dried over anhydrous Na₂SO₄, and evaporated to clear oil. The oil was chromatographed by eluting with gradient 2.5% to 4% MeOH in CHCl₃ to give the title compound as a clear oil (151 mg, 39%).

¹H NMR (500 MHz, CDCl₃) δ ppm 1.00 (t, J=7.32 Hz, 3H) 1.75-1.85 (m, 1H) 2.05-2.14 (m, 1H) 2.57-2.61 (m, 4H) 2.85 (t, J=5.61 Hz, 2H) 3.73-3.76 (m, 4H) 4.19 (t, J=5.61 Hz, 2H) 5.36 (dd, J=6.83, 4.39 Hz, 1H) 6.86 (d, J=1.95 Hz, 1H) 7.03 (dd, J=8.54, 2.20 Hz, 1H) 7.79 (d, J=8.30 Hz, 1H).

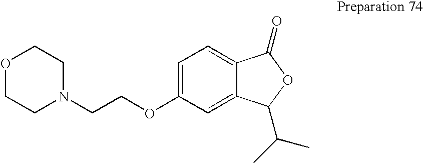

### Preparation of 3-isopropyl-5-(2-morpholin-4-yl-ethoxy)-3H-isobenzofuran-1-one

To a solution of N,N,N′,N′-tetramethylethylenediamine (0.211 ml, 1.4 mmol) in THF (10.0 ml) at −78° C. was added 1.4M s-BuLi/cyclohexane solution (1.0 ml, 1.4 mmol). Upon stirring for 5 minutes, N,N-diethyl-4-(2-morpholin-4-yl-ethoxy)-benzamide (408 mg, 1.33 mmol) in THF (2.0 ml) was added over 8 minutes. After stirring 15 minutes, isobutyraldehyde (0.157 ml, 1.73 mmol) was added and 5 minutes later the cold bath removed allowing the reaction to warm to room temperature. After 15 minutes, 10% HCl aqueous solution (10 ml) was added and the mixture stirred for 1 hour. The solution was then basified by adding saturated NaHCO₃ solution, and extracted with EtOAc. The combined organic layers were washed with H₂O, brine, dried over anhydrous Na₂SO₄, and evaporated to clear oil. The oil was chromatographed by eluting with gradient 1% to 3% MeOH in CHCl₃ to give the title compound as a clear oil (222 mg, 55%).

¹H NMR (500 MHz, CDCl₃) δ ppm 0.80 (d, J=6.83 Hz, 3H) 1.13 (d, J=6.83 Hz, 3H) 2.20-2.28 (m, 1H) 2.57-2.62 (m, 4H) 2.85 (t, J=5.61 Hz, 2H) 3.73-3.76 (m, 4H) 4.19 (t, J=5.61 Hz, 2H) 5.28 (d, J=3.91 Hz, 1H) 6.87 (d, J=1.95 Hz, 1H) 7.03 (dd, J=8.30, 1.95 Hz, 1H) 7.79 (d, J=8.30 Hz, 1H).

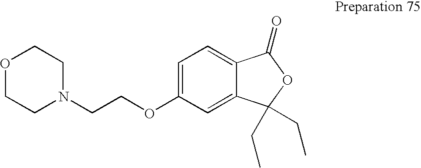

### Preparation of 3,3-diethyl-5-(2-morpholin-4-yl-ethoxy)-3H-isobenzofuran-1-one

Experimental procedure similar to Preparation 74.

Obtained a clear oil (233 mg, 55%).

¹H NMR (500 MHz, CDCl₃) δ ppm 0.72 (t, J=7.32 Hz, 6H) 1.87 (dq, J=14.65, 7.32 Hz, 2H) 2.03-2.11 (m, 2H) 2.58-2.62 (m, 4H) 2.85 (t, J=5.61 Hz, 2H) 3.73-3.76 (m, 4H) 4.19 (t, J=5.61 Hz, 2H) 6.75 (d, J=1.95 Hz, 1H) 7.01 (dd, J=8.54, 2.20 Hz, 1H) 7.76 (d, J=8.30 Hz, 1H).

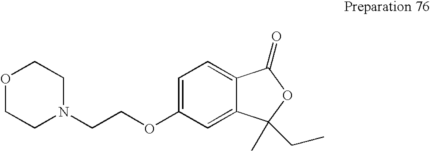

### Preparation of 3-ethyl-3-methyl-5-(2-morpholin-4-yl-ethoxy)-3H-isobenzofuran-1-one

Experimental procedure similar to Preparation 74.

¹H NMR (500 MHz, CDCl₃) δ ppm 0.76 (t, J=7.32 Hz, 3H) 1.61 (s, 3H) 1.83-1.91 (m, 1H) 2.04 (dq, J=14.65, 7.32 Hz, 1H) 2.57-2.63 (m, 4H) 2.85 (t, J=5.61 Hz, 2H) 3.73-3.76 (m, 4H) 4.19 (t, J=5.61 Hz, 2H) 6.78 (d, J=1.95 Hz, 1H) 7.00 (dd, J=8.54, 2.20 Hz, 1H) 7.77 (d, J=8.30 Hz, 1H).

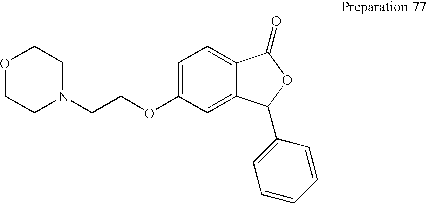

### Preparation of 5-(2-morpholin-4-yl-ethoxy)-3-phenyl-3H-isobenzofuran-1-one

Experimental procedure similar to Preparation 74.

¹H NMR (500 MHz, CDCl₃) δ ppm 2.53-2.56 (m, 4H) 2.78 (t, J=5.61 Hz, 2H) 3.69-3.71 (m, 4H) 4.09-4.13 (m, 2H) 6.30 (s, 1H) 6.73 (d, J=1.95 Hz, 1H) 7.05 (dd, J=8.30, 1.95 Hz, 1H) 7.26-7.29 (m, 2H) 7.37-7.40 (m, 3H) 7.86 (d, J=8.30 Hz, 1H).

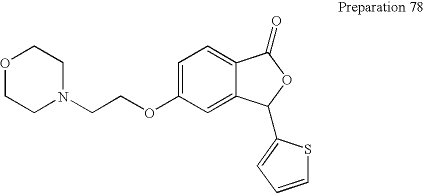

### 5-(2-Morpholin-4-yl-ethoxy)-3-thiophen-2-yl-3H-isobenzofuran-1-one Prepared in a Experimental procedure similar to Preparation 74

¹H NMR (500 MHz, CDCl₃) δ ppm 2.55-2.58 (m, 4H) 2.82 (t, J=5.61 Hz, 2H) 3.71-3.73 (m, 4H) 4.16 (t, J=5.61 Hz, 2H) 6.57 (s, 1H) 6.87 (d, J=2.44 Hz, 1H) 7.03 (dd, J=4.88, 3.42 Hz, 1H) 7.09 (dd, J=8.54, 2.20 Hz, 1H) 7.16 (d, J=3.42 Hz, 1H) 7.37 (dd, J=4.88, 0.98 Hz, 1H) 7.85 (d, J=8.30 Hz, 1H).

### Example 497

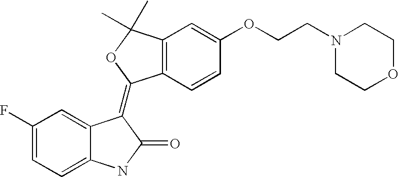

### 3-[3,3-Dimethyl-5-(2-morpholin-4-yl-ethoxy)-3H-isobenzofuran-1-ylidene]-5-fluoro-1,3-dihydro-indol-2-one

To a solution of 5-fluorooxindole (103 mg, 0.68 mmol) in dimethoxyethane (3.0 ml) at 0° C. was added 1.0M LiHMDS/THF (1.4 ml). The mixture was stirred for 2 minutes at 0° C., and then the ice bath removed. After 5 minutes, 3,3-dimethyl-5-(2-morpholin-4-yl-ethoxy)-3H-isobenzofuran-1-one (142 mg, 0.49 mmol) in dimethoxyethane (1.0 ml) was added and the reaction mixture stirred at room temperature for 4 hours. The reaction was quenched with 4% HCl aqueous solution (40 ml) and after 5 minutes H₂O (20 ml) added. The solution was filtered and the precipitate rinsed with H₂O to give a yellow solid. The solid was dissolved in EtOAc and the organic layer washed with saturated NaHCO₃, H₂O, brine, dried over anhydrous Na₂SO₄, and evaporated to a yellow solid. The solid was triturated with MeOH to give the title compound as a yellow solid (75 mg, 36%).

¹H NMR (500 MHz, DMSO-d6) δ ppm 1.72 (s, 6H) 2.50 (hidden s, 4H) 2.74 (t, J=5.86 Hz, 2H) 3.58-3.61 (m, 4H) 4.24 (t, J=5.61 Hz, 2H) 6.77 (dd, J=8.54, 4.64 Hz, 1H) 6.90 (ddd, J=9.76, 8.30, 2.44 Hz, 1H) 7.13 (dd, J=8.79, 2.44 Hz, 1H) 7.31 (d, J=2.44 Hz, 1H) 7.55 (dd, J=9.52, 2.68 Hz, 1H) 9.50 (d, J=8.79 Hz, 1H) 10.37 (s, 1H).

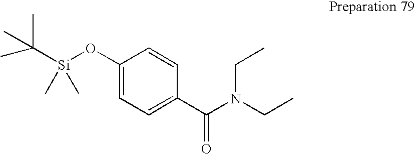

### Preparation of 4-(tert-Butyl-dimethyl-silanyloxy)-N,N-diethyl-benzamide

A solution of N,N-diethyl-4-hydroxy-benzamide (1.02 g, 5.26 mmol), triethylamine (1.5 ml, 10.5 mmol), and tert-butyldimethylsilyl chloride (1.03 g, 6.83 mmol) in DMF (15 ml) was stirred at room temperature for 1.5 hours. The mixture was partitioned between EtOAc and H₂O. The EtOAc layer was washed with H₂O, dilute aqueous HCl, H₂O, brine, dried over anhydrous Na₂SO₄, and evaporated to a light yellow solid. The solid was chromatographed by eluting with gradient 20% to 30% EtOAc in hexane to give 4-(tert-butyl-dimethyl-silanyloxy)-N,N-diethyl-benzamide as a white solid (1.28 g, 79%).

¹H NMR (500 MHz, CDCl₃) δ ppm 0.20 (s, 6H) 0.98 (s, 9H) 1.17 (broad s, 6H) 3.40 (broad d, 4H) 6.82-6.85 (m, 2H) 7.26-7.29 (m, 2H).

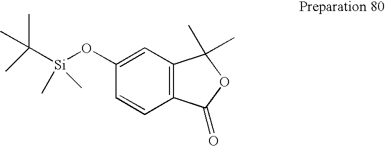

### 5-(tert-Butyl-dimethyl-silanyloxy)-3,3-dimethyl-3H-isobenzofuran-1-one

To a solution of N,N,N′,N′-tetramethylethylenediamine (0.14 ml, 0.95 mmol) in THF (10.0 ml) at −78° C. was added 1.4M s-BuLi/cyclohexane solution (0.80 ml). Upon stirring for 10 minutes, 4-(tert-butyl-dimethyl-silanyloxy)-N,N-diethyl-benzamide (265 mg, 0.86 mmol) in THF (0.5 ml) was added over 10 minutes. After stirring 40 minutes, acetone (0.127 ml, 1.72 mmol) was added and 15 minutes later the cold bath removed allowing the reaction to warm to room temperature. The reaction was quenched with 10% HCl (6 ml) and stirred at room temperature for 0.15 minutes. The mixture was partitioned between EtOAc and H₂O. The EtOAc layer was washed with H₂O, dilute aqueous NaOH, H₂O, dilute aqueous HCl, brine, dried over anhydrous Na₂SO₄, and evaporated to light yellow oil. The oil was crystallized from hexane to give the title compound as a white solid (90 mg, 36%).

¹H NMR (500 MHz, CDCl₃) δ ppm 0.26 (s, 6H) 1.00 (s, 9H) 1.63 (s, 6H) 6.75 (d, J=1.95 Hz, 1H) 6.92 (dd, J=8.30, 2.44 Hz, 1H) 7.72 (d, J=8.30 Hz, 1H).

### Example 498

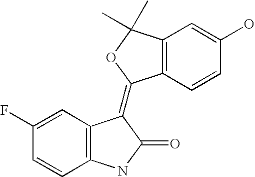

### 5-Fluoro-3-(5-hydroxy-3,3-dimethyl-3H-isobenzofuran-1-ylidene)-1,3-dihydro-indol-2-one

To a solution of 5-fluorooxindole (131 mg, 0.87 mmol) in THF (3.0 ml) at 0° C. was added 1.0M LiHMDS/THF (1.7 ml). After the ice bath was removed, the solution was stirred for 5 minutes at room temperature. 5-(Tert-butyl-dimethyl-silanyloxy)-3,3-dimethyl-3H-isobenzofuran-1-one (127 mg, 0.43 mmol) in THF (0.5 ml) was added and the reaction mixture stirred at room temperature for 1.5 hours. The reaction was quenched with 10% HCl aqueous solution (8 ml), stirred for 16 hours, and then partitioned between EtOAc and H₂O. The EtOAc layer was washed with H₂O, brine, dried over anhydrous Na₂SO₄, and evaporated to yellow oil. The oil was dissolved in THF (10 ml), followed by the addition of 10% HCl aqueous solution (3.0 ml), and then the mixture was heated at 50□C for 3.5 hours. The mixture was partitioned between EtOAc and H₂O. The EtOAc layer was washed with H₂O, brine, dried over anhydrous Na₂SO₄, and evaporated to a yellow solid. The solid was recrystallized from EtOAc/hexane and then from CHCl₃/MeOH to give the title compound as a yellow solid (78 mg, 58%).

¹H NMR (500 MHz, CDCl₃) δ ppm 1.69 (s, 6H) 6.76 (dd, J=8.30, 4.88 Hz, 1H) 6.88 (ddd, J=9.76, 8.54, 2.69 Hz, 1H) 6.93-6.96 (m, 2H) 7.54 (dd, J=9.52, 2.69 Hz, 1H) 9.43-9.46 (m, 1H) 10.33 (s, 1H) 10.66 (broad s, 1H).

### Example 499

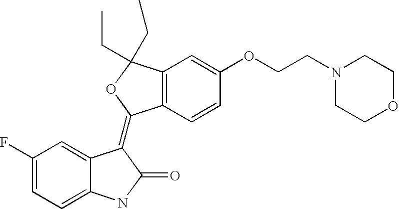

### 3-[3,3-Diethyl-5-(2-morpholin-4-yl-ethoxy)-3H-isobenzofuran-1-ylidene]-5-fluoro-1,3-dihydro-indol-2-one

Experimental procedure similar to Example 497.

¹H NMR (500 MHz, DMSO-d6) δ ppm 0.62 (t, J=7.32 Hz, 6H) 2.04 (dq, J=14.65, 7.32 Hz, 2H) 2.20 (tq, J=7.32, 7.16 Hz, 2H) 2.50 (hidden s, 4H) 2.74 (t, J=5.86 Hz, 2H) 3.58-3.61 (m, 4H) 4.24 (t, J=5.86 Hz, 2H) 6.77-6.80 (m, 1H) 6.90 (ddd, J=9.64, 8.42, 2.44 Hz, 1H) 7.14 (dd, J=9.03, 2.20 Hz, 1H) 7.20 (d, J=2.44 Hz, 1H) 7.49 (dd, J=9.52, 2.69 Hz, 1H) 9.51 (d, J=9.28 Hz, 1H) 10.39 (s, 1H).

### Example 500

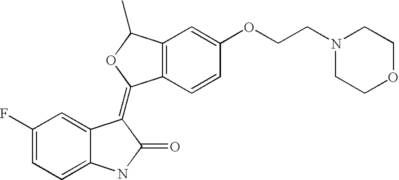

### 5-Fluoro-3-[3-methyl-5-(2-morpholin-4-yl-ethoxy)-3H-isobenzofuran-1-ylidene]-1,3-dihydro-indol-2-one

To a solution of 5-fluorooxindole (73 mg, 0.48 mmol) in dimethoxyethane (2.0 ml) cooled to 0° C. was added 1.0M LiHMDS/THF (1.1 ml) over 3 minutes. The mixture was stirred for 3 minutes at 0° C., and then the ice bath removed. After 7 minutes, 3-methyl-5-(2-morpholin-4-yl-ethoxy)-3H-isobenzofuran-1-one (120 mg, 0.43 mmol) in dimethoxyethane (0.5 ml) was added and the reaction mixture stirred at room temperature for 2.5 hours. The reaction was quenched with 4% HCl aqueous solution and then basified with saturated NaHCO₃. The mixture was extracted with EtOAc and the organic layer washed with H₂O, brine, dried with anhydrous Na₂SO₄, and evaporated to a yellow solid. The solid was recrystallized from EtOAc/MeOH to give the title compound as a yellow solid (41 mg, 23%).

¹H NMR (500 MHz, DMSO-d6) δ ppm 1.69 (d, J=6.35 Hz, 3H) 2.50 (hidden s, 4H) 2.74 (t, J=5.61 Hz, 2H) 3.57-3.61 (m, 4H) 4.20-4.28 (m, 2H) 6.01 (q, J=6.35 Hz, 1H) 6.78 (dd, J=8.30, 4.88 Hz, 1H) 6.88-6.92 (m, 1H) 7.15 (dd, J=9.03, 2.20 Hz, 1H) 7.26 (d, J=1.95 Hz, 1H) 7.55 (dd, J=9.52, 2.69 Hz, 1H) 9.53 (d, J=8.79 Hz, 1H) 10.38 (s, 1H).

### Example 501

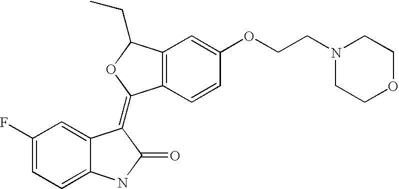

### 3-[3-Ethyl-5-(2-morpholin-4-yl-ethoxy)-3H-isobenzofuran-1-ylidene]-5-fluoro-1,3-dihydro-indol-2-one

To a solution of 5-fluorooxindole (78 mg, 0.52 mmol) in dimethoxyethane (2.0 ml) cooled to 0° C. was added 1.0M LiHMDS/THF (1.1 ml) over 3 minutes. The mixture was stirred for 3 minutes at 0° C., and then the ice bath removed. After 7 minutes, 3-ethyl-5-(2-morpholin-4-yl-ethoxy)-3H-isobenzofuran-1-one (135 mg, 0.46 mmol) in dimethoxyethane (0.5 ml) was added and the reaction mixture stirred at room temperature for 4 hours. The reaction was quenched with 4% HCl aqueous solution and then washed with EtOAc. The EtOAc layer was extracted with 4% HCl aqueous solution. The combined acidic aqueous layers were washed with EtOAc, and then basified with saturated NaHCO₃. The basic aqueous layer was extracted with EtOAc and the organic layer washed with H₂O, brine, dried over anhydrous Na₂SO₄, and evaporated to a yellow oily solid. The solid was triturated with CHCl₃ to give 3-[3-ethyl-5-(2-morpholin-4-yl-ethoxy)-3H-isobenzofuran-1-ylidene]-5-fluoro-1,3-dihydro-indol-2-one as a yellow solid (9 mg, 4%).

¹H NMR (500 MHz, DMSO-d6) δ ppm 0.96 (t, J=7.32 Hz, 3H) 1.78-1.86 (m, 1H) 2.24-2.32 (m, 1H) 2.50 (hidden s, 4H) 2.74 (t, J=5.61 Hz, 2H) 3.56-3.61 (m, 4H) 4.20-4.28 (m, 2H) 5.92 (dd, J=6.83, 3.91 Hz, 1H) 6.79 (dd, J=8.30, 4.88 Hz, 1H) 6.88-6.93 (m, 1H) 7.15 (dd, J=8.79, 1.95 Hz, 1H) 7.25 (s, 1H) 7.51 (dd, J=9.76, 2.44 Hz, 1H) 9.53 (d, J=8.79 Hz, 1H) 10.39 (s, 1H).

### Example 502

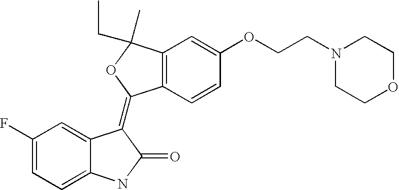

### 3-[3-Ethyl-3-methyl-5-(2-morpholin-4-yl-ethoxy)-3H-isobenzofuran-1-ylidene]-5-fluoro-1,3-dihydro-indol-2-one

To a solution of 5-fluorooxindole (88 mg, 0.58 mmol) in THF (3.0 ml) at 0° C. was added 1.0M LiHMDS/THF (1.2 ml). The mixture was stirred for 2 minutes at 0° C., and then the ice bath removed. After 5 minutes, 3-ethyl-3-methyl-5-(2-morpholin-4-yl-ethoxy)-3H-isobenzofuran-1-one (127 mg, 0.42 mmol) in THF (0.8 ml) was added and the reaction mixture stirred at room temperature for 2.5 hours. The reaction was quenched with 4% HCl aqueous solution (40 ml) and after 5 minutes H₂O (50 ml) added. The aqueous portion was decanted away from the gummy solid. The solid was dissolved in EtOAc and the organic layer washed with saturated NaHCO₃, H₂O, brine, dried over anhydrous Na₂SO₄, and evaporated to a yellow-brown solid. The solid was recrystallized from EtOAc/hexane to give 3-[3-ethyl-3-methyl-5-(2-morpholin-4-yl-ethoxy)-3H-isobenzofuran-1-ylidene]-5-fluoro-1,3-dihydro-indol-2-one as a yellow solid (12 mg, 7%).

¹H NMR (500 MHz, DMSO-d6) δ ppm 0.68 (t, J=7.32 Hz, 3H) 1.70 (s, 3H) 1.97-2.05 (m, 1H) 2.14-2.22 (m, J=7.32, 7.32, 7.32, 7.32, 7.32 Hz, 1H) 2.50 (hidden s, 4H) 2.74 (t, J=5.61 Hz, 2H) 3.56-3.62 (m, 4H) 4.24 (t, J=5.61 Hz, 2H) 6.78 (dd, J=8.54, 4.64 Hz, 1H) 6.90 (ddd, J=9.76, 8.54, 2.69 Hz, 1H) 7.13 (dd, J=8.79, 2.44 Hz, 1H) 7.25 (d, J=2.44 Hz, 1H) 7.51 (dd, J=9.52, 2.69 Hz, 1H) 9.50 (d, J=8.79 Hz, 1H) 10.38 (s, 1H).

### Example 503

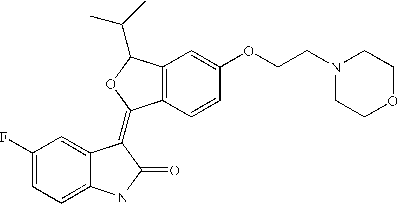

### 5-Fluoro-3-[3-isopropyl-5-(2-morpholin-4-yl-ethoxy)-3H-isobenzofuran-1-ylidene]-1,3-dihydro-indol-2-one

Experimental procedure similar to Example 502.

¹H NMR (500 MHz, DMSO-D6) δ ppm 0.60 (d, J=6.83 Hz, 3H) 1.24 (d, J=6.83 Hz, 3H) 2.42-2.47 (m, 1H) 2.50 (hidden s, 4H) 2.74 (t, J=5.61 Hz, 2H) 3.57-3.60 (m, 4H) 4.20-4.28 (m, 2H) 5.87 (d, J=2.93 Hz, 1H) 6.79 (dd, J=8.54, 4.64 Hz, 1H) 6.89-6.94 (m, 1H) 7.16 (dd, J=8.79, 1.95 Hz, 1H) 7.22 (d, J=1.95 Hz, 1H) 7.47 (dd, J=9.76, 2.93 Hz, 1H) 9.54 (d, J=8.79 Hz, 1H) 10.41 (s, 1H).

### Example 504

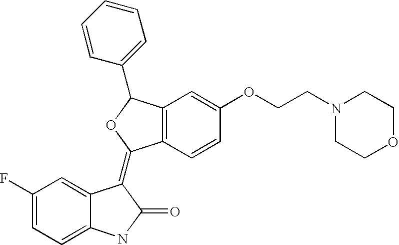

### 5-Fluoro-3-[5-(2-morpholin-4-yl-ethoxy)-3-phenyl-3H-isobenzofuran-1-ylidene]-1,3-dihydro-indol-2-one

Experimental procedure similar to Example 497.

¹H NMR (500 MHz, DMSO-d6) δ ppm 2.44 (s, 4H) 2.68 (t, J=5.61 Hz, 2H) 3.52-3.56 (m, 4H) 4.13-4.20 (m, 2H) 6.78-6.82 (m, 1H) 6.88-6.93 (m, 1H) 6.99 (d, J=2.44 Hz, 1H) 7.02 (s, 1H) 7.19 (dd, J=8.79, 2.44 Hz, 1H) 7.38-7.47 (m, 6H) 9.60 (d, J=8.79 Hz, 1H) 10.47 (s, 1H).

### Example 505

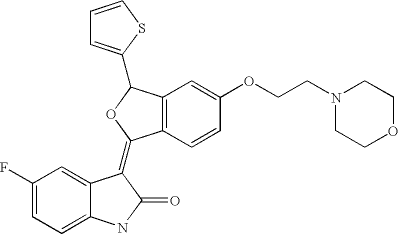

Experimental procedure similar to Example 497.

¹H NMR (500 MHz, DMSO-d6) δ ppm 2.46 (s, 4H) 2.71 (t, J=5.61 Hz, 2H) 3.55-3.58 (m, 4H) 4.17-4.24 (m, 2H) 6.79 (dd, J=8.30, 4.39 Hz, 1H) 6.91 (ddd, J=9.64, 8.42, 2.44 Hz, 1H) 7.11 (d, J=1.95 Hz, 1H) 7.14 (dd, J=5.13, 3.66 Hz, 1H) 7.23 (dd, J=9.03, 2.20 Hz, 1H) 7.33 (s, 1H) 7.42-7.45 (m, 2H) 7.65 (dd, J=5.13, 1.22 Hz, 1H) 9.57 (d, J=8.79 Hz, 1H) 10.47 (s, 1H).

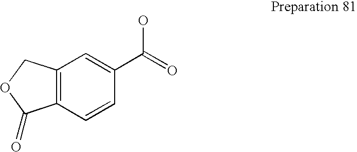

### Preparation of 1-oxo-1,3-dihydro-isobenzofuran-5-carboxylic acid

To a stirred suspension of 5-cyanophthalide (2.5 g, 15.7 mmol) in 10 ml of water was added 15 ml of 5M NaOH solution. After the mixture was heated in a 100° C.-bath for 4 hours, additional water (80 ml) was added. The mixture was cooled to 60° C., and acidified with 8 ml of concentrated HCl. After heated again at 100° C. for 20 minutes, the mixture was cooled with ice. The resulting white precipitates were filtered, washed with water, and dried under vacuum to give the title compound as a white solid (2.68 g, 96%).

¹H NMR (500 MHz, DMSO-d6) δ ppm 5.48 (s, 2H) 7.95 (d, J=8.30 Hz, 1H) 8.11 (d, J=7.81 Hz, 1H) 8.22 (s, 1H) 13.53 (s, 1H)

### Example 506

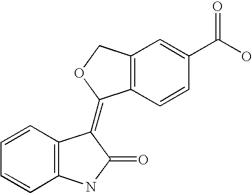

### 1-(2-Oxo-1,2-dihydro-indol-3-ylidene)-1,3-dihydro-isobenzofuran-5-carboxylic acid

To a stirred solution of oxindole (447 mg, 3.4 mmol) in anhydrous tetrahydrofuran (20 ml) under nitrogen was added 1.0M LiHMDS/THF solution (6.7 ml, 6.7 mmol). The mixture was stirred at room temperature for 10 minutes, and 1-oxo-1,3-dihydro-isobenzofuran-5-carboxylic acid (200 mg, 1.1 mmol) was added. After stirring at room temperature for 2 hours, the mixture was poured into 2M HCl aqueous solution (10 ml) and heated at 65° C. for 1 hour. The mixture was cooled to room temperature, and then poured into water (200 ml) with vigorous stirring. The resulting solid was filtered, rinsed with water and dried under vacuum to give the title compound as yellow solids (149 mg, 45%).

¹H NMR (500 MHz, DMSO-d6) δ ppm 5.86 (s, 2H) 6.84 (d, J=7.32 Hz, 1H) 6.98 (t, J=7.57 Hz, 1H) 7.14 (t, J=8.30 Hz, 1H) 7.86 (d, J=7.81 Hz, 1H) 8.10 (d, J=9.76 Hz, 1H) 8.18 (s, 1H) 9.72 (d, J=8.30 Hz, 1H) 10.51 (s, 1H) 13.36 (s, 1H)

### Example 507

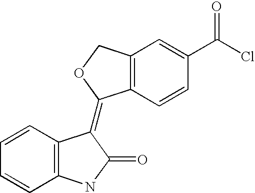

### 1-(2-Oxo-1,2-dihydro-indol-3-ylidene)-1,3-dihydro-isobenzofuran-5-carbonyl chloride

To a stirred suspension of 1-(2-oxo-1,2-dihydro-indol-3-ylidene)-1,3-dihydro-isobenzofuran-5-carboxylic acid (35 mg, 0.12 mmol) in Et₂O (5 ml) was added 2M oxalyl chloride/CH₂Cl₂ solution (0.5 ml, 1 mmol), followed by addition of DMF (one drop). The reaction was stirred for 1 hour, filtered, washed with Et₂O, and then dried to give the title compound as a brown solid (37 mg, 100%).

### Example 508

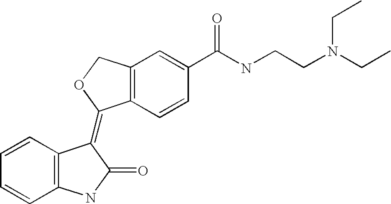

### 1-(2-Oxo-1,2-dihydro-indol-3-ylidene)-1,3-dihydro-isobenzofuran-5-carboxylic acid (2-diethylamino-ethyl)-amide

A mixture of 1-(2-oxo-1,2-dihydro-indol-3-ylidene)-1,3-dihydro-isobenzofuran-5-carboxylic acid (100 mg, 0.34 mmol) and 1,1′-carbonyldiimidazole (100 mg, 0.62 mmol) in THF (10 ml) was stirred at room temperature for 16 hours. N,N-diethylethylenediamine (0.25 ml, 1.76 mmol) was added. The mixture was stirred for 15 minutes and poured into water (100 ml). The precipitates were filtered, washed with water and dried under vacuum to give thte title compound as a yellow powder (99 mg, 74%).

¹H NMR (500 MHz, DMSO-d6) δ ppm 0.98 (t, J=7.08 Hz, 6H) 2.53 (t, J=7.08 Hz, 4H) 2.58 (t, J=7.32 Hz, 2H) 3.36 (t, J=7.32 Hz, 2H) 5.86 (s, 2H) 6.84 (d, J=7.32 Hz, 1H) 6.98 (td, J=7.69, 1.22 Hz, 1H) 7.14 (td, J=7.57, 1.46 Hz, 1H) 7.85 (d, J=7.32 Hz, 1H) 7.98 (d, J=8.79 Hz, 1H) 8.05 (s, 1H) 8.62 (t, J=5.86 Hz, 1H) 9.68 (d, J=8.30 Hz, 1H) 10.49 (s, 1H)

The following Examples 509-511 were prepared using the experiment procedure described in Example 508, but with the appropriate reagent, reaction conditions and reactant substitutions that will be readily realized by those of ordinary skill in this art without the exercise of undue experimentation

### Example 509

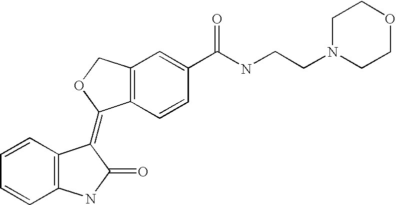

### 1-(2-Oxo-1,2-dihydro-indol-3-ylidene)-1,3-dihydro-isobenzofuran-5-carboxylic acid (2-morpholin-4-yl-ethyl)-amide

¹H NMR (500 MHz, DMSO-d6) δ ppm 2.43 (br s, 4H) 2.50 (br s, 2H) 3.43 (br s, 2H) 3.58 (br s, 4H) 5.86 (s, 2H) 6.84 (d, J=7.81 Hz, 1H) 6.98 (t, J=7.57 Hz, 1H) 7.14 (t, J=7.81 Hz, 1H) 7.85 (d, J=7.32 Hz, 1H) 7.98 (d, J=8.30 Hz, 1H) 8.06 (s, 1H) 8.65 (s, 1H) 9.68 (d, J=8.30 Hz, 1H) 10.48 (s, 1H)

### Example 510

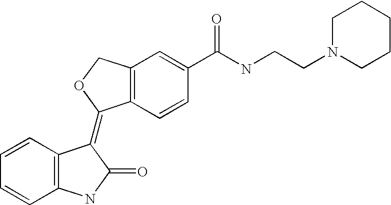

### 1-(2-Oxo-1,2-dihydro-indol-3-ylidene)-1,3-dihydro-isobenzofuran-5-carboxylic acid (2-piperidin-1-yl-ethyl)-amide

¹H NMR (500 MHz, DMSO-d6) δ ppm 1.34-1.42 (m, 2H) 1.47-1.53 (m, 4H) 2.39 (br. s., 4H) 2.46 (t, J=7.08 Hz, 2H) 3.41 (q, J=6.35 Hz, 2H) 5.86 (s, 2H) 6.84 (d, J=7.81 Hz, 1H) 6.98 (t, J=8.05 Hz, 1H) 7.14 (t, J=7.08 Hz, 1H) 7.85 (d, J=7.32 Hz, 1H) 7.97 (d, J=8.30 Hz, 1H) 8.05 (s, 1H) 8.62 (t, J=5.61 Hz, 1H) 9.68 (d, J=8.30 Hz, 1H) 10.48 (s, 1H)

### Example 511

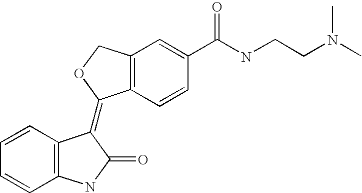

### 1-(2-Oxo-1,2-dihydro-indol-3-ylidene)-1,3-dihydro-isobenzofuran-5-carboxylic acid (2-dimethylamino-ethyl)-amide

### Example 512

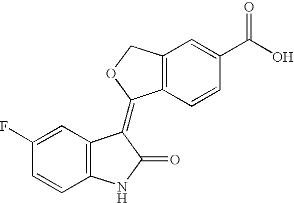

### 1-(5-Fluoro-2-oxo-1,2-dihydro-indol-3-ylidene)-1,3-dihydro-isobenzofuran-5-carboxylic acid

To a solution of 5-fluorooxindole (3.7 g, 24.5 mmol) in anhydrous tetrahydrofuran (55 ml) under nitrogen was treated with 1.0M LiHMDS/THF solution (70 ml, 70 mmol). The resulting mixture was stirred at room temperature for 10 minutes and 1-oxo-1,3-dihydro-isobenzofuran-5-carboxylic acid (1.4 g, 7.9 mmol) was added. Having stirred at room temperature for several hours at room temperature, the mixture was poured into 2M HCl aqueous solution and heated at 70° C. for 1 hour. The mixture was cooled to room temperature, and then poured into water with vigorous stirring. The resulting solid was filtered, rinsed with water and dried under vacuum the title compound as a yellow solid (2.29 g, 94%).

1H NMR (500 MHz, DMSO-d6) δ ppm 5.82 (s, 2H) 6.75 (dd, J=8.30, 4.88 Hz, 1H) 6.87-6.97 (m, 1H) 7.55 (dd, J=9.52, 2.69 Hz, 4H) 8.05 (d, J=8.30 Hz, 1H) 8.14 (s, 4H) 9.65 (d, J=8.30 Hz, 4H) 10.47 (s, 1H) 13.33 (s, 1H)

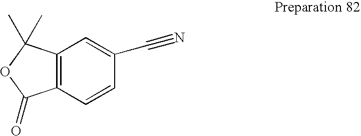

### Preparation of 3,3-dimethyl-1-oxo-1,3-dihydro-isobenzofuran-5-carbonitrile

To a solution of 5-cyanophthalide (5.0 g) in anhydrous DMF (110 ml) in an ice-bath was added sodium hydride in mineral oil (60%, 2.77 g, 69.2 mmol) in portion. After stirred at the same temperature for 10 minutes, methyl iodide was injected. The resulting mixture was stirred for 16 hours from 0° C. to room temperature. The mixture was poured into a mixture of aqueous 1M HCl and chloroform in an ice-bath, and then stirred for one hour. The organic layer was washed with brine, dried over anhydrous magnesium sulfate. Removal of the solvent led to a yellowish residue. Purification of the residue through chromatography with elute of hexane and ethyl acetate to give the title compound as a white solid (1.75 g, 30%).

1H NMR (500 MHz, CDCl₃) δ ppm 1.71 (s, 6H) 7.73 (s, 1H) 7.82 (d, J=7.81 Hz, 1H) 8.00 (d, J=7.81 Hz, 1H)

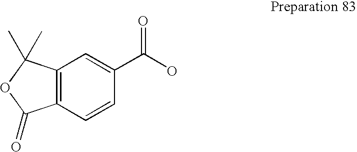

### Preparation of 3,3-dimethyl-1-oxo-1,3-dihydro-isobenzofuran-5-carboxylic acid

A suspension of 3,3-dimethyl-1-oxo-1,3-dihydro-isobenzofuran-5-carbonitrile (0.411 g, 2.20 mmol) in aqueous 5M NaOH (5.0 ml) was heated at 100° C. for 20 minutes, and then the reaction mixture became a clean solution. After the mixture was cooled to room temperature, water was added (4 ml). The mixture was cooled in an ice-bath, concentrated HCl (4 ml) was added and a white suspension deposited. The suspension was warmed to room temperature, and then heated at 100° C. for 20 minutes. After cooled to room temperature, the white suspension was cooled further in an ice bath. The resulting white precipitate was filtered, washed with water, and dried under vacuum to give the title compound as a white solid (0.414 g, 92%).

1H NMR (500 MHz, DMSO-d6) δ ppm 1.66 (s, 6H) 7.90 (d, J=7.81 Hz, 8H) 8.10 (d, J=7.81 Hz, 1H) 8.30 (s, 1H) 13.56 (s, 1H)

### Example 513

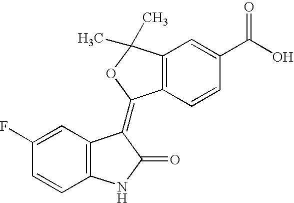

### 1-(5-Fluoro-2-oxo-1,2-dihydro-indol-3-ylidene)-3,3-dimethyl-1-3-dihydro-isobenzofuran-5-carboxylic acid

To a solution of 5-fluorooxindole (3.7 g, 24.5 mmol) in anhydrous tetrahydrofuran (1.0 ml) under nitrogen was treated with 1.0M LiHMDS/THF solution (2.4 ml, 2.4 mmol). The resulting mixture was stirred at room temperature for 10 minutes and 3,3-dimethyl-1-oxo-1,3-dihydro-isobenzofuran-5-carboxylic acid (50 mg, 42 mmol) was added. The resulting solution was stirred at room temperature for several hours at room temperature. The mixture was poured into 2M HCl aqueous solution and heated at 70° C. for 1 hour. The mixture was cooled to room temperature, and then poured into water with vigorous stirring. The resulting solid was filtered, rinsed with water to give a crude solid. The crude solid was dissolved in a mixture of MeOH/DMF, and then water was added. The resulting solid was filtered, rinsed with water, and dried under vacuum to give the title compound as a yellow solid (44 mg, 58%).

1H NMR (500 MHz, DMSO-d6) δ ppm 1.71 (s, 6H) 6.61-6.81 (m, 2H) 6.86-6.99 (m, 1H) 7.57 (dd, J=9.28, 2.44 Hz, 1H) 8.05 (d, J=8.30 Hz, 1H) 8.16 (s, 1H) 9.61 (d, J=8.30 Hz, 1H) 10.46 (s, 1H) 13.37 (s, 1H)

### Example 514

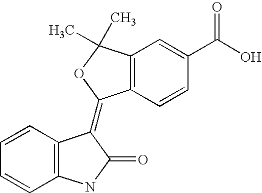

### 3,3-Dimethyl-1-(2-oxo-1,2-dihydro-indol-3-ylidene)-1,3-dihydro-isobenzofuran-5-carboxylic acid

Experimental procedure similar to Example 513.

### Example 515

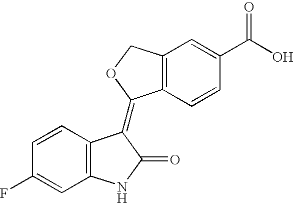

### 1-(6-Fluoro-2-oxo-1,2-dihydro-indol-3-ylidene)-1,3-dihydro-isobenzofuran-5-carboxylic acid

Experimental procedure similar to Example 513.

The following Examples 516-520 were prepared using the experiment procedure described in Example 508, but with the appropriate reagent, reaction conditions and reactant substitutions that will be readily realized by those of ordinary skill in this art without the exercise of undue experimentation

### Example 516

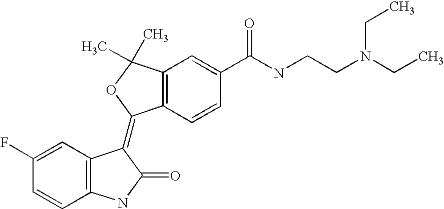

### 1-(5-Fluoro-2-oxo-1,2-dihydro-indol-3-ylidene)-3,3-dimethyl-1,3-dihydro-isobenzofuran-5-carboxylic acid (2-diethylamino-ethyl)-amide

1H NMR (500 MHz, CDCl₃) δ ppm 1.16 (t, J=7.49 Hz, 6H) 1.71 (m, 6H) 2.76-2.87 (m, 4H) 2.92 (s, 2H) 3.66-3.72 (m, 2H) 6.73-6.79 (m, 1H) 6.83-6.89 (m, 1H) 7.66 (dd, J=9.52, 2.68 Hz, 1H) 7.89 (s, 1H) 7.95 (d, J=8.30 Hz, 1H) 8.34 (s, 1H) 9.01 (s, 1H) 9.66 (d, J=8.30 Hz, 1H)

### Example 517

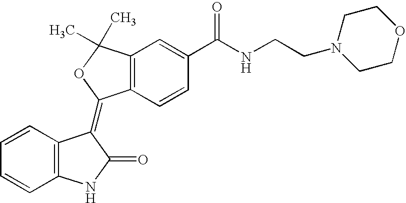

### 3,3-Dimethyl1-(2-Oxo-1,2-dihydro-indol-3-ylidene)-1,3-dihydro-isobenzofuran-5-carboxylic acid (2-morpholin-4-yl-ethyl)-amide

Experimental procedure similar to Example 508.

1H NMR (500 MHz, CDCl₃) δ ppm 1.70 (s, 6H) 2.59 (s, 4H) 2.67-2.73 (m, 2H) 3.65 (q, J=5.37 Hz, 2H) 3.71-3.78 (m, 4H) 6.84 (d, J=7.32 Hz, 1H) 7.06 (t, J=7.32 Hz, 1H) 7.15-7.20 (m, 1H) 7.37 (s, 1H) 7.81 (d, J=8.30 Hz, 1H) 7.83 (s, 1H) 7.96 (d, J=6.83 Hz, 1H) 8.36 (s, 1H) 9.69 (d, J=8.30 Hz, 1H)

### Example 518

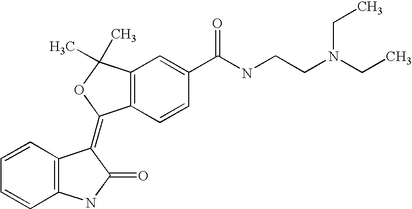

### 3,3-Dimethyl-1-(2-oxo-1,2-dihydro-indol-3-ylidene)-1,3-dihydro-isobenzofuran-5-carboxylic acid (2-diethylamino-ethyl)-amide

Experimental procedure similar to Example 508.

1H NMR (500 MHz, CDCl₃) δ ppm 1.08 (t, J=7.08 Hz, 2H) 2.68 (q, J=7.00 Hz, 4H) 2.75-2.82 (m, 2H) 3.55-3.64 (m, 2H) 6.84 (d, J=7.81 Hz, 1H) 7.05 (t, J=7.08 Hz, 1H) 7.16 (t, J=8.30 Hz, 1H) 7.84 (s, 1H) 7.90 (d, J=8.30 Hz, 1H) 7.94-8.00 (m, 2H) 8.93 (s, 1H) 9.69 (d, J=8.30 Hz, 1H)

### Example 519

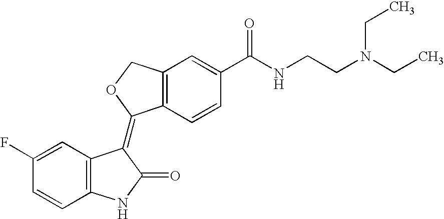

### 1-(5-Fluoro-2-oxo-1,2-dihydro-indol-3-ylidene)-1,3-dihydro-isobenzofuran-5-carboxylic acid (2-diethylamino-ethyl)-amide

Experimental procedure similar to Example 508.

1H NMR (500 MHz, DMSO-d6) δ ppm 0.92 (t, J=7.08 Hz, 6H) 2.32-2.60 (m, 6H) 3.14-3.39 (m, 2H) 5.82 (s, 2H) 6.75 (dd, J=8.30, 4.39 Hz, 1H) 6.83-6.97 (m, 1H) 7.45-7.60 (m, 1H) 7.92 (d, J=8.79 Hz, 1H) 8.01 (s, 1H) 8.56 (t, J=5.37 Hz, 1H) 9.61 (d, J=8.30 Hz, 1H) 10.44 (s, 1H)

### Example 520

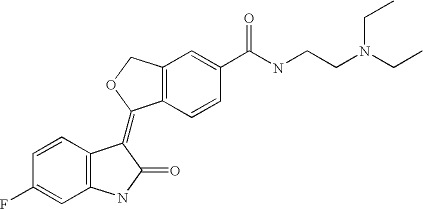

### 1-(6-Fluoro-2-oxo-1,2-dihydro-indol-3-ylidene)-1,3-dihydro-isobenzofuran-5-carboxylic acid (2-diethylamino-ethyl)-amide

Experimental procedure similar to Example 508.

1H NMR (500 MHz, DMSO-d6) δ ppm 0.97 (t, J=7.08 Hz, 6H) 2.45-2.63 (m, 6H) 3.30-3.39 (m, 2H) 5.84 (s, 2H) 6.65 (dd, J=9.28, 1.95 Hz, 1H) 6.74-6.82 (m, 1H) 7.81 (dd, J=8.30, 5.86 Hz, 1H) 7.96 (d, J=8.30 Hz, 1H) 8.04 (s, 1H) 8.59 (t, J=5.61 Hz, 1H) 9.61 (d, J=8.30 Hz, 1H) 10.62 (s, 1H)

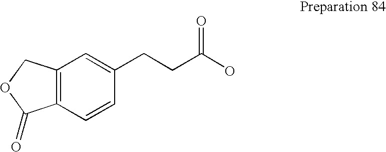

### Preparation of 3-(1-oxo-1,3-dihydro-isobenzofuran-5-yl)-propionic acid

To a stirred solution of 5-(3-hydroxy-propyl)-3H-isobenzofuran-1-one (738 mg, 3.84 mmol) in acetone (30 ml), was added 5 ml of 2M HCl solution, followed by dropwise addition of a solution of KMnO4 (910 mg, 5.76 mmol) in 30 ml of water. The mixture was stirred at room temperature for 1 hour and filtered through celite. The filtrate was concentrated, and then the separated precipitates were filtered, washed with water, and dried in vacuo give the title compound as a white powder (610 mg, 77%).

¹H NMR (500 MHz, DMSO-d6) δ ppm 2.62 (t, J=7.57 Hz, 2H) 2.97 (t, J=7.57 Hz, 2H) 5.38 (s, 2H) 7.47 (d, J=8.79 Hz, 1H) 7.54 (s, 1H) 7.76 (d, J=8.30 Hz, 1H) 12.21 (br. s., 1H)

### Example 521

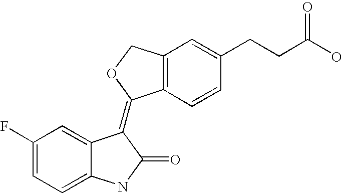

### 3-[1-(5-Fluoro-2-oxo-1,2-dihydro-indol-3-ylidene)-1,3-dihydro-isobenzofuran-5-yl]-propionic acid

To a stirred solution of 5-fluorooxindole (218 mg, 1.44 mmol) in anhydrous THF (10 ml) under nitrogen was added 1.0M LiHMDS/THF solution (2.9 ml, 2.9 mmol). After the mixture was stirred at room temperature for 10 minutes, 3-(1-Oxo-1,3-dihydro-isobenzofuran-5-yl)-propionic acid (100 mg, 0.48 mmol) was added. After the mixture was stirred at room temperature for 2 hours, 1M sulfuric acid solution (10 ml) was added. The mixture was heated at 60° C. for 2 hours, and then poured into 150 ml of water. The resulting solid was filtered, rinsed with water, dried in vacuo to give the title compound as a yellow solid (96 mg, 59%).

¹H NMR (500 MHz, DMSO-d6) δ ppm 2.63 (t, J=7.57 Hz, 2H) 2.98 (t, J=7.57 Hz, 2H) 5.80 (s, 2H) 6.79 (dd, J=8.54, 4.64 Hz, 1H) 6.90-6.96 (m, 1H) 7.47 (d, J=8.30 Hz, 1H) 7.54 (s, 1H) 7.57 (dd, J=9.76, 2.44 Hz, 1H) 9.54 (d, J=8.30 Hz, 1H) 10.42 (s, 1H) 12.21 (s, 1H)

### Example 522

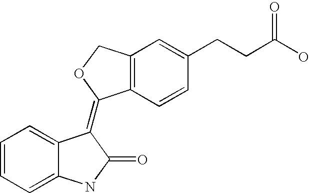

### 3-[1-(2-Oxo-1,2-dihydro-indol-3-ylidene)-1,3-dihydro-isobenzofuran-5-yl]-propionic acid

Experimental procedure similar to Example 521.

¹H NMR (500 MHz, DMSO-d6) δ ppm 2.63 (t, J=7.57 Hz, 2H) 2.97 (t, J=7.57 Hz, 2H) 5.77 (s, 2H) 6.82 (d, J=7.81 Hz, 1H) 6.95 (t, J=7.81 Hz, 1H) 7.10 (t, J=8.30 Hz, 1H) 7.45 (d, J=8.30 Hz, 1H) 7.52 (s, 1H) 7.82 (d, J=7.81 Hz, 1H) 9.55 (d, J=8.30 Hz, 1H) 10.39 (s, 1H) 12.20 (s, 1H)

### Example 523

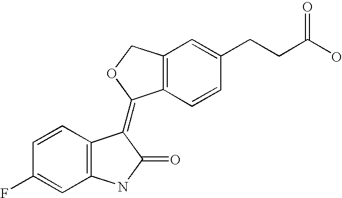

### 3-[1-(6-Fluoro-2-oxo-1,2-dihydro-indol-3-ylidene)-1,3-dihydro-isobenzofuran-5-yl]-propionic acid

Experimental procedure similar to Example 521.

¹H-NMR (500 MHz, DMSO-d6) δ ppm 2.62 (t, J=7.57 Hz, 2H) 2.97 (t, J=7.57 Hz, 2H) 5.77 (s, 2H) 6.63 (dd, J=9.03, 2.20 Hz, 1H) 6.73-6.79 (m, 1H) 7.45 (d, J=8.79 Hz, 1H) 7.52 (s, 1H) 7.79 (dd, J=8.30, 5.86 Hz, 1H) 9.49 (d, J=7.81 Hz, 1H) 10.55 (s, 1H) 12.20 (s, 1H)

### Example 524

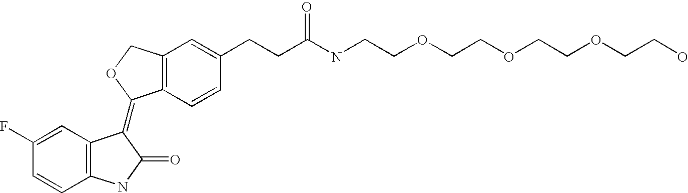

### 3-[1-(5-Fluoro-2-oxo-1,2-dihydro-indol-3-ylidene)-1,3-dihydro-isobenzofuran-5-yl]-N-(2-{2-[2-(2-hydroxy-ethoxy)-ethoxy]-ethoxy}-ethyl)-propionamide

To a stirred suspension of 3-[1-(5-Fluoro-2-oxo-1,2-dihydro-indol-3-ylidene)-1,3-dihydro-isobenzofuran-5-yl]-propionic acid (200 mg, 0.59 mmol) in CH₂Cl₂ (10 ml), was added oxalyl chloride (0.20 ml, 2.3 mmol). The mixture was cooled to 0° C. and one drop of DMF was added. The mixture was stirred at room temperature for 30 min and evaporated to dryness. The resulting orange solid was suspended in THF (10 ml), cooled to 0° C., and then triethylamine (0.5 ml) was added, followed by addition of 1-amino-3,6,9-trioxaundecanyl-11-ol (300 mg, 1.5 mmol). The mixture was allowed to warm to room temperature and stirred for 30 minutes. The mixture was partitioned between brine (50 ml) and EtOAc (50 ml). The aqueous layer was extracted with EtOAc (2×50 ml). The organic layers were combined, washed with 0.5M HCl (2×50 ml) and saturated NaHCO₃ (50 ml), dried over Na₂SO₄, and concentrated. Purification of the mixture through silica gel chromatography with a gradient of MeOH in CHCl₃ led to give the title compound as a yellow solid (103 mg, 34%).

¹H NMR (500 MHz, DMSO-d6) δ ppm 2.46 (t, J=7.57 Hz, 2H) 2.93-3.00 (m, 2H) 3.19 (q, J=5.86 Hz, 2H) 3.35-3.41 (m, 4H) 3.45-3.50 (m, 10H) 4.57 (t, J=5.37 Hz, 1H) 5.80 (s, 2H) 6.79 (dd, J=8.54, 4.64 Hz, 1H) 6.93 (td, J=9.03, 2.44 Hz, 1H) 7.43 (d, J=8.79 Hz, 1H) 7.50 (s, 1H) 7.58 (dd, J=9.76, 2.44 Hz, 1H) 7.94 (t, J=5.61 Hz, 1H) 9.53 (d, J=8.30 Hz, 1H) 10.42 (s, 1H)

MS (ES+): 515.2194 (MH⁺), 537.1990 (M+Na⁺)

MS (ES−): 513.2081 (M−H)

### Example 525

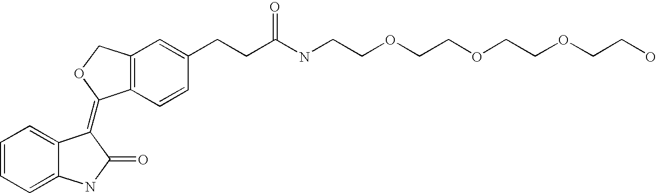

### N-(2-{2-[2-(2-Hydroxy-ethoxy)-ethoxy]-ethoxy}-ethyl)-3-[1-(2-oxo-1,2-dihydro-indol-3-ylidene)-1,3-dihydro-isobenzofuran-5-yl]-propionamide

Experimental procedure similar to Example 524.

MS (ES+): 519.2304 (M+Na⁺)

### Example 526

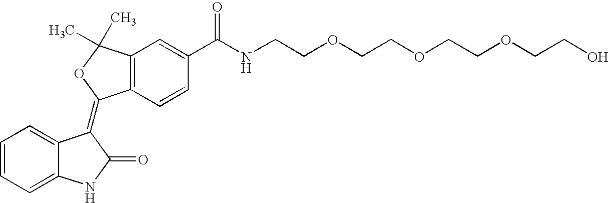

### 3,3-Dimethyl-1-(2-oxo-1,2-dihydro-indol-3-ylidene)-1,3-dihydro-isobenzofuran-5-carboxylic acid (2-{2-[(2-hydroxy-ethoxy)-ethoxy]-ethoxy}-ethyl)-amide

Experimental procedure similar to Example 524.

1H NMR (500 MHz, CDCl₃) δ ppm 1.76 (s, 6H) 3.60-3.75 (m, 16H) 6.87 (d, J=7.81 Hz, 1H) 7.07 (t, J=7.08 Hz, 1H) 7.14-7.20 (m, 1H) 7.63 (s, 1H) 7.95 (s, 1H) 7.99-8.01 (m, 2H) 8.17 (s, 1H) 9.71 (d, J=8.30 Hz, 1H)

### Example 527

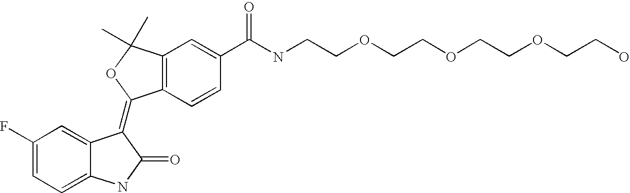

### 1-(5-Fluoro-2-oxo-1,2-dihydro-indol-3-ylidene)-3,3-dimethyl-1,3-dihydro-isobenzofuran-5-carboxylic acid (2-{2-[2-(2-hydroxy-ethoxy)-ethoxy]-ethoxy}-ethyl)-amide

Experimental procedure similar to Example 524.

1H NMR (500 MHz, CDCl₃) δ ppm 1.78 (s, 6H) 3.48-3.80 (m, 16H) 6.72-6.93 (m, 2H) 7.70 (d, J=9.28 Hz, 1H) 7.99 (s, 1H) 8.13 (d, J=7.81 Hz, 1H) 8.47 (s, 1H) 9.76 (d, J=8.30 Hz, 1H)(cheak again)

### Example 528

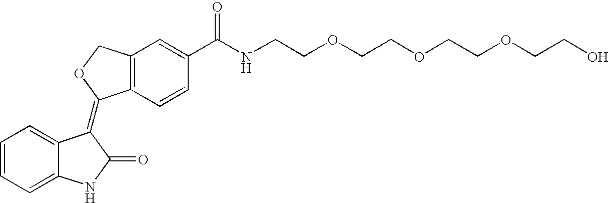

### 1-(2-Oxo-1,2-dihydro-indol-3-ylidene)-1,3-dihydro-isobenzofuran-5-carboxylic acid (2-{2-[2-(2-hydroxy-ethoxy)-ethoxy]-ethoxy}-ethyl)-amide

Experimental procedure similar to Example 524.

1H NMR (500 MHz, DMSO-d6) δ ppm 3.61-3.74 (m, 17H) 6.84-6.89 (m, J=7.81 Hz, 2H) 7.04-7.09 (m, 1H) 7.17 (d, J=7.81 Hz, 1H) 7.52-7.56 (m, 1H) 7.96-8.02 (m, 2H) 8.04 (s, 1H) 8.09-8.14 (m, 1H) 9.75 (d, J=8.30 Hz, 1H)

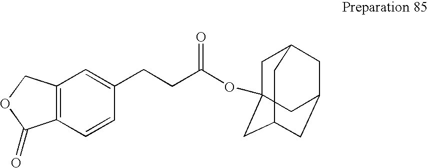

### Preparation of 3-(1-oxo-1,3-dihydro-isobenzofuran-5-yl)-propionic acid adamantan-1-yl ester

A mixture of 3-(1-oxo-1,3-dihydro-isobenzofuran-5-yl)-propionic acid (100 mg, 0.48 mmol), 1-adamantanol (146 mg, 0.96 mmol), DCC (109 mg, 0.53 mmol) and DMAP (5 mg) in anhydrous CH₂Cl₂ (10 ml) was stirred at room temperature for 16 hours. The mixture was filtered, concentrated and subjected to silica gel column chromatography. Elution with a gradient of EtOAc in hexanes gave 3-(1-oxo-1,3-dihydro-isobenzofuran-5-yl)-propionic acid adamantan-1-yl ester (72 mg, 44%) as white crystals.

1H NMR (500 MHz, CDCl₃) δ ppm 1.66 (s, 6H) 2.06 (d, J=2.44 Hz, 6H) 2.16 (s, 3H) 2.60 (t, J=7.57 Hz, 2H) 3.04 (t, J=7.57 Hz, 2H) 5.29 (s, 2H) 7.34 (s, 1H) 7.39 (d, J=7.81 Hz, 1H) 7.85 (d, J=7.81 Hz, 1H)

### Example 529

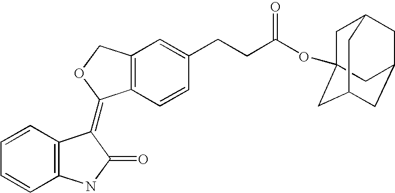

### 3-[1-(2-Oxo-1,2-dihydro-indol-3-ylidene)-1,3-dihydro-isobenzofuran-5-yl]-propionic acid adamantan-1-yl ester

To a stirred solution of oxindole (38 mg, 0.29 mmol) in anhydrous THF (5 ml) under nitrogen was added 1.0M LiHMDS/THF solution (0.58 ml, 0.58 mmol). The mixture was stirred at room temperature for 10 minutes, and 3-(1-oxo-1,3-dihydro-isobenzofuran-5-yl)-propionic acid adamantan-1-yl ester (50 mg, 0.15 mmol) was added. After stirring at room temperature for 2.5 hours, the mixture was quenched with 2 ml of 2M HCl solution, heated at 50° C. for 20 minutes and poured into 150 ml of water. The resulting solid was filtered, rinsed with water, dried in vacuo to give the title compound as a yellow solid (20 mg).

1H NMR (500 MHz, DMSO-d6) δ ppm 1.60 (s, 6H) 2.01 (d, J=2.44 Hz, 6H) 2.10 (s, 3H) 2.61 (t, J=7.57 Hz, 2H) 2.95 (t, J=7.32 Hz, 2H) 5.77 (s, 2H) 6.82 (d, J=7.32 Hz, 1H) 6.95 (t, J=7.57 Hz, 1H) 7.10 (t, J=7.57 Hz, 1H) 7.44 (d, J=8.79 Hz, 1H) 7.51 (s, 1H) 7.82 (d, J=7.81 Hz, 1H) 9.54 (d, J=8.30 Hz, 1H) 10.40 (s, 1H)

MS (ES+): 456.3736 (MH⁺), 478.3641 (M+Na⁺)

### Example 530

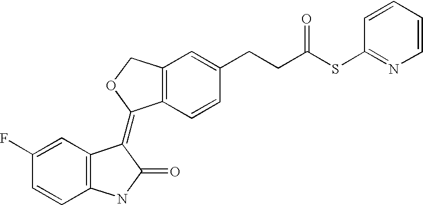

### 3-[1-(5-Fluoro-2-oxo-1,2-dihydro-indol-3-ylidene)-1,3-dihydro-isobenzofuran-5-yl]-thiopropionic acid S-pyridin-2-yl ester

A mixture of 3-[1-(5-fluoro-2-oxo-1,2-dihydro-indol-3-ylidene)-1,3-dihydro-isobenzofuran-5-yl]-propionic acid (100 mg, 0.29 mmol), 2-pyridyldisulfide (84 mg, 0.38 mmol) and triphenylphosphine (106 mg, 0.41 mmol) in CH₂Cl₂ (10 ml) was stirred under nitrogen for 18 hours. The mixture was concentrated and run through silica gel column with a gradient of MeOH in CHCl₃. The yellow fraction was collected and evaporated to give the title compound as a yellow solid (84 mg, 67%).

¹H NMR (500 MHz, DMSO-d6) δ ppm 3.08 (t, J=7.32 Hz, 2H) 3.19 (t, J=7.32 Hz, 2H) 5.80 (s, 2H) 6.79 (dd, J=8.30, 4.39 Hz, 1H) 6.89-6.96 (m, 1H) 7.44 (dd, J=7.57, 4.64 Hz, 1H) 7.49 (d, J=8.30 Hz, 1H) 7.55-7.59 (m, 2H) 7.61 (d, J=7.81 Hz, 1H) 7.85-7.93 (m, 1H) 8.55-8.64 (m, 1H) 9.54 (d, J=8.30 Hz, 1H) 10.42 (s, 1H)

### Example 531

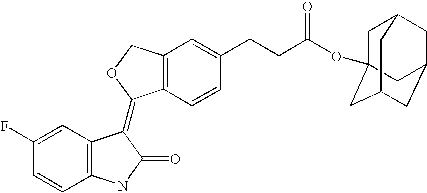

### 3-[1-(5-Fluoro-2-oxo-1,2-dihydro-indol-3-ylidene)-1,3-dihydro-isobenzofuran-5-yl]-propionic acid adamantan-1-yl ester

To a stirred suspension of 3-[1-(5-fluoro-2-oxo-1,2-dihydro-indol-3-ylidene)-1,3-dihydro-isobenzofuran-5-yl]-thiopropionic acid S-pyridin-2-yl ester (30 mg, 0.069 mmol) and copper(II) bromide (26 mg, 0.12 mmol) in anhydrous CH₃CN (3 ml), was added 1-adamantanol (30 mg, 0.20 mmol). The mixture was stirred at room temperature for 2 hours, and then poured into a mixture of saturated NH₄Cl solution (20 ml) and saturated NaHCO₃ solution (10 ml). The aqueous layer was extracted with CH₂Cl₂ (3×30 ml), the organic layers were combined, evaporated. Subjection of the residual mixture to silica gel column chromatography with elution of a gradient of MeOH in to give the title compound as a yellow solid (10 mg).

MS (ES+): 496.1906 (M+Na⁺)

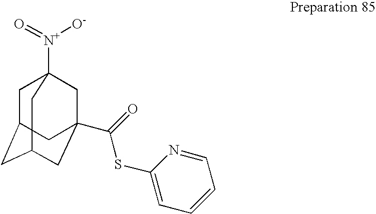

### Preparation of 3-Nitro-adamantane-1-carbothioic acid S-pyridin-2-yl ester

A mixture of 1-adamantanecarboxylic acid (2.00 g, 11.1 mmol), N-hydroxyphthalimide (181 mg, 1.1 mmol) and 70% nitric acid (1.4 ml, 22 mmol) in α,α.α-trifluorotoluene (20 ml) was purged with argon and heated in a 65° C.-bath for 14 hours. The mixture was concentrated under reduced pressure and purified through a short silica gel column with 20% MeOH/CHCl₃ to afford 2.1 g of crude 3-nitro-1-adamantanecarboxylic acid as semi-solid. To a solution of this semi-solid in CH₂Cl₂ (30 ml), was added 2-pyridyldisulfide (3.0 g, 13.6 mmol) and triphenylphosphine (4.1 g, 15.6 mmol). The mixture was stirred at room temperature under nitrogen for 16 hours, concentrated, and purified by silica gel chromatography eluted with a gradient of EtOAc in hexanes to give the title compound as light yellow oil (0.90 g, 25%).

¹H NMR (500 MHz, DMSO-d6) δ ppm 1.68 (s, 2H) 1.91 (s, 4H) 2.13-2.21 (m, 4H) 2.37 (s, 4H) 7.45 (dd, J=7.57, 4.64 Hz, 1H) 7.59 (dd, J=7.81, 0.98 Hz, 1H) 7.89 (td, J=7.81, 1.95 Hz, 1H) 8.61 (dt, J=4.88, 0.98 Hz, 1H)

### Example 532

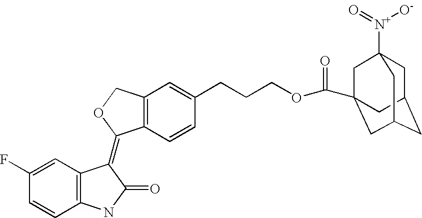

### 3-Nitro-adamantane-1-carboxylic acid 3-[1-(5-fluoro-2-oxo-1,2-dihydro-indol-3-ylidene)-1,3-dihydro-isobenzofuran-5-yl]-propyl ester

A mixture of 3-nitro-adamantane-1-carbothioic acid S-pyridin-2-yl ester (730 mg, 2.29 mmol), 5-fluoro-3-[5-(3-hydroxy-propyl)-3H-isobenzofuran-1-ylidene]-1,3-dihydro-indol-2-one (820 mg, 2.52 mmol), and copper(II) bromide (563 mg, 2.52 mmol) in 30 ml of anhydrous acetonitrile was stirred in 70° C. bath under nitrogen for 80 minutes. The mixture was cooled to room temperature and poured into a mixture of saturated NH₄Cl solution (70 ml) and saturated NaHCO₃ solution (35 ml). The precipitates were filtered, washed with water, dried under vacuum to afford the crude product, which was purified by silica gel chromatography eluted with a gradient of MeOH in CHCl₃ to give the title compound as yellow solid (400 mg, 33%).

¹H NMR (500 MHz, DMSO-d6) δ ppm 1.61 (s, 2H) 1.70-1.80 (m, 4H) 1.94-2.01 (m, 2H) 2.05-2.15 (m, 4H) 2.22 (s, 2H) 2.26 (s, 2H) 2.81 (t, J=7.32 Hz, 2H) 4.07 (t, J=6.35 Hz, 2H) 5.79 (s, 2H) 6.78 (dd, J=8.30, 4.39 Hz, 1H) 6.92 (s, 1H) 7.44 (d, J=8.30 Hz, 1H) 7.53 (s, 1H) 7.57 (dd, J=9.76, 2.44 Hz, 1H) 9.54 (d, J=8.30 Hz, 1H) 10.41 (s, 1H)

### Example 533

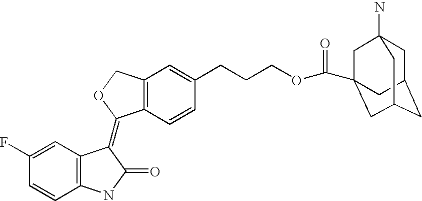

### 3-Amino-adamantane-1-carboxylic acid 3-[1-(5-fluoro-2-oxo-1,2-dihydro-indol-3-ylidene)-1,3-dihydro-isobenzofuran-5-yl]-propyl ester

To a stirred suspension of 3-nitro-adamantane-1-carboxylic acid 3-[1-(5-fluoro-2-oxo-1,2-dihydro-indol-3-ylidene)-1,3-dihydro-isobenzofuran-5-yl]-propyl ester (400 mg, 0.75 mmol) in 10 ml of AcOH was added zinc powder (500 mg, 7.7 mmol). The mixture was stirred under nitrogen at room temperature for 5 hours and filtered through celite. The filtrate was concentrated to dryness. The resulting yellow solids were mixed with 500 mg of NaHCO₃ powder and purified by silica gel chromatography with a gradient of MeOH in CHCl₃ to give the title compound as a yellow solid (293 mg, 78%).

¹H NMR (500 MHz, DMSO-d6) δ ppm 1.52 (d, J=10.74 Hz, 1H) 1.59 (d, J=12.69 Hz, 1H) 1.66 (d, J=11.72 Hz, 2H) 1.69 (s, 4H) 1.75 (d, J=11.72 Hz, 2H) 1.84 (s, 2H) 1.93-1.99 (m, 2H) 2.16 (s, 2H) 2.80 (t, J=7.32 Hz, 2H) 4.05 (t, J=6.35 Hz, 2H) 5.81 (s, 2H) 6.79 (dd, J=8.54, 4.64 Hz, 1H) 6.92 (td, J=9.15, 2.68 Hz, 1H) 7.4 (br.s., 2H) 7.43 (d, J=8.79 Hz, 1H) 7.53 (s, 1H) 7.57 (dd, J=9.76, 2.44 Hz, 1H) 9.55 (d, J=8.30 Hz, 1H) 10.42 (s, 1H)

### Example 534

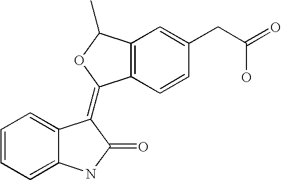

### [3-Methyl-1-(2-oxo-1,2-dihydro-indol-3-ylidene)-1,3-dihydro-isobenzofuran-5-yl]-acetic acid

MS (ES+): 322.1058 (MH⁺)

### Example 535

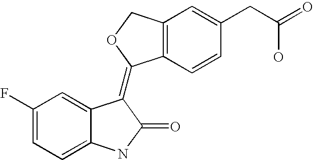

### [1-(5-Fluoro-2-oxo-1,2-dihydro-indol-3-ylidene)-1,3-dihydro-isobenzofuran-5-yl]-acetic acid

### Example 536

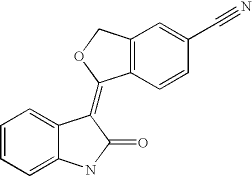

### 1-(2-Oxo-1,2-dihydro-indol-3-ylidene)-1,3-dihydro-isobenzofuran-5-carbonitrile

To a stirred solution of oxindole (1.67 g, 12.5 mmol) in anhydrous THF (20 ml) under nitrogen was added 1M LiHMDS/THF solution (25 ml, 25 mmol). The mixture was stirred at room temperature for 10 minutes, and then 5-cyanophthalide (1.00 g, 6.28 mmol) was added. The mixture was stirred at room temperature for 1.5 hours, poured into 2M HCl solution (25 ml) and heated at 75° C. for 1 hour. The mixture was poured into 400 ml of water with stirring. The resulting precipitates were filtered, washed with water, and dried under vacuum to give the title compound as a yellow solid (850 mg, 49%).

¹H NMR (500 MHz, DMSO-d6) δ ppm 5.86 (s, 2H) 6.85 (d, J=7.32 Hz, 1H) 6.99 (t, J=7.57 Hz, 1H) 7.16 (t, J=7.57 Hz, 1H) 7.85 (d, J=7.32 Hz, 1H) 8.05 (d, J=8.79 Hz, 1H) 8.16 (s, 1H) 9.80 (d, J=8.30 Hz, 1H) 10.56 (s, 1H)

### Example 537

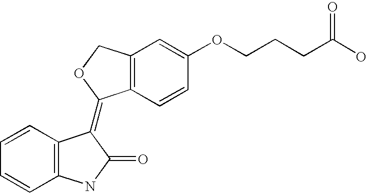

### 4-[1-(2-Oxo-1,2-dihydro-indol-3-ylidene)-1,3-dihydro-isobenzofuran-5-yloxy]-butyric acid

¹H NMR (500 MHz, DMSO-d6) δ ppm 1.95-2.03 (m, 2H) 2.41 (t, J=7.32 Hz, 2H) 4.12 (t, J=6.59 Hz, 2H) 5.75 (s, 2H) 6.81 (d, J=7.81 Hz, 1H) 6.94 (t, J=7.57 Hz, 1H) 7.07 (td, J=7.57, 1.46 Hz, 1H) 7.12 (dd, J=8.79, 2.44 Hz, 1H) 7.22 (d, J=1.95 Hz, 1H) 7.79 (d, J=7.81 Hz, 1H) 9.57 (d, J=8.79 Hz, 1H) 10.35 (s, 1H)

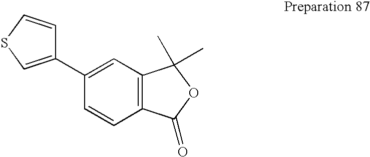

### Preparation of 3,3-dimethyl-5-thiophen-3-yl-3H-isobenzofuran-1-one

A mixture of trifluoromethanesulfonic acid 3,3-dimethyl-1-oxo-1,3-dihydro-isobenzofuran-5-yl ester (310 mg, 1.0 mmol), 3-thiopheneboronic acid (141 mg, 1.1 mmol), potassium fluoride (192 mg, 3.3 mmol), palladium(II)acetate (2.2 mg, 0.01 mmol), and 2-(dicyclohexylphosphino)biphenyl (4.2 mg, 0.012 mmol) in THF (2.0 ml) was stirred at room temperature for 10 minutes. The mixture was taken up in EtOAc and washed with 2% HCl aqueous solution, H₂O, dilute NaOH, 2% HCl, brine, dried with anhydrous Na₂SO₄ and evaporated to give dark oil. The oil was chromatographed to give 3,3-dimethyl-5-thiophen-3-yl-3H-isobenzofuran-1-one as an off-white solid (194 mg, 79%).

¹H NMR (500 MHz, CDCl₃) δ ppm 1.70 (s, 6H) 7.43-7.45 (m, 1H) 7.45-7.47 (m, 1H) 7.55-7.56 (m, 1H) 7.61 (dd, J=2.93, 1.10 Hz, 1H) 7.73 (dd, J=8.06, 1.46 Hz, 1H) 7.88 (dd, J=8.06, 0.73 Hz, 1H).

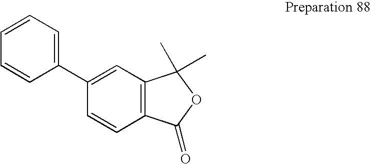

### Preparation of 3,3-dimethyl-5-phenyl-3H-isobenzofuran-1-one

Experimental procedure similar to Preparation 87.

¹H NMR (500 MHz, CDCl₃) δ ppm 1.71 (s, 6H) 7.42-7.46 (m, 1H) 7.48-7.52 (m, 2H) 7.55 (d, J=1.46 Hz, 1H) 7.61-7.64 (m, 2H) 7.72 (dd, J=7.81, 1.46 Hz, 1H) 7.91-7.93 (m, 1H).

### Example 538

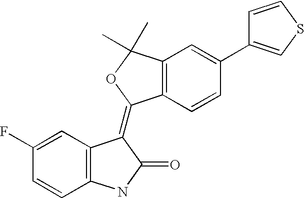

### 3-(3,3-Dimethyl-5-thiophen-3-yl-3H-isobenzofuran-1-ylidene)-5-fluoro-1,3-dihydro-indol-2-one

To a solution of 5-fluorooxindole (113 mg, 0.75 mmol) in THF (3.0 ml) at 0° C. was added 1.0M LiHMDS/THF (1.6 ml) over 1.5 minutes. After the ice bath was removed, the solution was stirred for 7 minutes at room temperature, and then the solid, 3,3-dimethyl-5-thiophen-3-yl-3H-isobenzofuran-1-one (91 mg, 0.37 mmol), was added in one portion. The reaction mixture was rapidly stirred at room temperature for 2 hours, and then quenched into 4% HCl aqueous solution (20 ml). THF was added until a clear solution was obtained, and then the mixture was stirred at room temperature for 15 minutes. Then H₂O was added to the solution to give a precipitate, which was filtered and rinsed with H₂O. The solid was dissolved in EtOAc, washed with H₂O, brine, dried with anhydrous Na₂SO₄, and evaporated to a yellow solid. The solid was recrystallized from EtOAc/hexane to give 3-(3,3-dimethyl-5-thiophen-3-yl-3H-isobenzofuran-1-ylidene)-5-fluoro-1,3-dihydro-indol-2-one as a yellow solid (83 mg, 59%).

¹H NMR (500 MHz, CDCl₃) δ ppm 1.79 (s; 6H) 6.77-6.80 (m, 1-H) 6.84-6.88 (m, 1H) 7.45-7.47 (m, 1H) 7.48 (dd, J=4.88, 1.46 Hz, 1H) 7.51 (d, J=0.98 Hz, 1H) 7.63 (dd, J=2.93, 1.46 Hz, 1H) 7.71 (dd, J=9.76, 2.44 Hz, 1H) 7.77 (dd, J=8.30, 1.95 Hz, 1H) 7.80 (broad s, 1H) 9.69 (d, J=8.30 Hz, 1H).

### Example 539

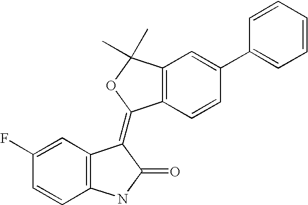

### 3-(3,3-Dimethyl-5-phenyl-3H-isobenzofuran-1-ylidene)-5-fluoro-1,3-dihydro-indol-2-one

Experimental procedure similar to Example 538.

¹H NMR (500 MHz, DMSO-d6) δ ppm 1.80 (s, 6H) 6.81 (dd, J=8.42, 4.39 Hz, 1H) 6.95 (ddd, J=9.70, 8.42, 2.75 Hz, 1H) 7.45-7.48 (m, 1H) 7.52-7.56 (m, 2H) 7.62 (dd, J=9.52, 2.93 Hz, 1H) 7.85 (dt, J=8.42, 1.65 Hz, 2H) 7.92 (dd, J=8.42, 1.83 Hz, 1H) 8.04-8.05 (m, 1H) 9.65 (d, J=8.42 Hz, 1H) 10.46 (s, 1H).

The present invention is not to be limited in scope by the exemplified embodiments which are intended as illustrations of single aspects of the invention only. Indeed, various modifications of the invention in addition to those described herein will become apparent to those skilled in the art from the foregoing description. For example novel compounds of formula II, below may be utilized in the method of treating diseases described above. 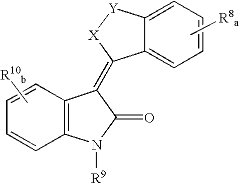
wherein X is O; Y is [C(R⁹)₂]_{c}; R¹⁰ is selected from the group consisting of halogen, nitro, hydroxy, hydrocarbyl, substituted hydrocarbyl, amide, thioamide, amine, thioether and sulfonyl; R⁸ is selected from the group consisting of halogen, nitro, hydroxy, hydrocarbyl, substituted hydrocarbyl, amide, thioamide, amine, thioether and sulfonyl and phosphonic acid; R⁹ is selected from the group consisting of hydrogen, hydrocarbyl and substituted hydrocarbyl; c is an integer of from 1 to 2; b is 0 or an integer from 1 to 3; a is 0 or an integer of from 1 to 3 and pharmaceutically acceptable salts thereof. Said hydrocarbyl and/or substituted hydrocarbyl may be alkyl, alkenyl, alkynyl, aryl (including carbocylic aryl and heterocyclic aryl) and alkaryl.

In a preferred embodiment of the invention (as represented by the novel compounds of formula II and the use thereof):
R¹⁰ is R¹⁴
R⁹ is R¹⁶
R⁸ is R¹²

In this embodiment:

Preferably a is 1 or a is 0 and Y is HCCOOH.

Preferably b is 0 or 1.

Preferably c is 1.

Preferably R¹⁴ is fluoro or chloro.

Preferably R¹⁶ is independently selected from the group consisting of hydrogen, carboxylic, lower alkyl, phenyl, thienyl, tetrazolyl, morpholinyl-N-methyl; more preferably R¹⁶ is hydrogen or methyl.

Preferably R¹² is selected from the group consisting of 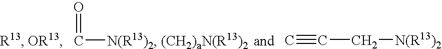
wherein R¹³ is selected from the group consisting of alkyl radicals, e.g. methyl, ethyl, or propyl radicals, or phenyl radicals which alkyl or phenyl radicals may be substituted with heteroatoms selected from the group consisting of halogen, nitrogen, oxygen and sulfur radicals or said phenyl radicals may be substituted with alkyl radicals.

In a first embodiment of the invention, R¹² is R¹³ wherein R¹³ is selected from the group consisting of alkyl radicals, e.g. methyl, ethyl or propyl radicals, which are substituted with heteroatoms selected from the group consisting of nitrogen, oxygen and sulfur.

Such heteroatoms may be pendant from said alkyl radicals or enchained. That is, for example said oxygen atom substituent may be included as a hydroxy radical, keto radical, an ether radical or as a sulfoxide radical. Preferably, in the first embodiment of this invention, R¹³ may include 1, 2, 3, 4 or 5 oxygen atoms, and/or 1 nitrogen atom and/or 1 sulfur atom.

For example, in said first embodiment, R¹³ may be selected from the group consisting of hydroxypropyl, methylsulfoxypropyl, aminopropyl, acetyl, 2,2-dimethoxyethyl, 2-(1,2 dioxyethylenyl)ethyl, hydroxyethenyl, carboxyethyl and N-ethylene oxide adducts of ethylcarbamic acid.

In a second embodiment of the invention, R¹² is OR¹³ wherein R¹³ is selected from the group consisting of alkyl radicals, e.g. methyl, ethyl or propyl radicals, which are substituted with heteroatoms selected from the group consisting of nitrogen and oxygen-containing radicals.

Such heteroatoms may be pendant from said alkyl radicals or enchained. Most preferably, in this second embodiment, R¹³ is N-ethyl morpholinyl.

In a third embodiment of the invention, R¹² is 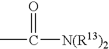
wherein R¹³ is selected from the group consisting of alkyl radicals, e.g. methyl, ethyl or propyl radicals, which are substituted with heteroatoms selected from the group consisting of nitrogen and oxygen containing radicals.

Such heteroatoms may be pendant from said alkyl radicals or enchained. Most preferably, in this third embodiment R¹³ is selected from the group consisting of 2-(diethylamino)ethyl, 2-(dimethylamino)ethyl, 2-(N-morpholinyl)ethyl, 2-(N-piperidinyl)ethyl and N-ethylene oxide adducts, e.g. hydroxyl(ethyloxy)ₐethyl, wherein a is an integer of from 1 to 3, e.g. 3.

In a fourth embodiment of the invention R¹² is (CH₂)ₐN(R¹³)₂. In this embodiment, when a is 0, R¹² is N(R¹³)₂ wherein R¹³ is selected from the group consisting of alkyl radicals e.g. methyl, ethyl or propyl radicals, which are substituted with heteroatoms selected from the group consisting of nitrogen or oxygen-containing radicals or halogen, e.g. chloro, radicals. Such heteroatoms may be pendant from said alkyl radicals or enchained.

More preferably, N(R¹³)₂ is N(Et)₂ wherein Et is ethyl and may be substituted with one chloro or hydroxy radical, or 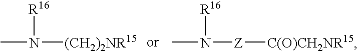
wherein Z is CH₂—CH₂—O or is absent and R¹⁶ is H or methyl and R¹⁵ together with N forms a N-cycloalkyl radical, e.g. piperidine, which may be substituted with one or more lower alkyl radicals, e.g. methyl radicals, or halogen radicals, e.g. fluoro radicals; more preferably up to one methyl or fluoro radical, and/or said cycloalkyl radical can include an additional enchained nitrogen atom or an oxygen atom, e.g. as in morpholine or piperazine. Most preferably, in this fourth embodiment preferably one of R¹³ is ethyl or 2-hydroxyethyl or 2-chloroethyl, or NR¹⁵ is selected from the group consisting of N-piperidyl, N-morpholinyl, N(4-methyl piperazyl), N-(3,5 dimethyl morpholinyl), and N-(4-fluoropiperidyl).

Alternatively, in this embodiment, a may be 1, 2 or 3 and R¹² is (CH₂)ₐN(R¹³)₂ or (CH₂)ₐNR¹⁵ wherein R¹³ is selected from the group consisting of phenyl radicals, alkyl-substituted phenyl radicals, alkyl radicals, e.g. methyl, ethyl and propyl radicals, which alkyl radicals may be substituted with hetero atoms selected from the group consisting of nitrogen, oxygen or sulfur radicals and R¹⁵, together with N, forms a N-cycloalkyl radical, e.g. piperidine or pyrollidine, which may be substituted with alkyloxyalkyl, carboxyalkyl, sulfonic acid, hydroxy or carboxy radicals and/or said cycloalkyl radical can include an additional enchained nitrogen atom or an oxygen or a sulfur atom, e.g. as in morpholine, piperazine or 1,4 sulfone hexazane.

Most preferably, in this alternative of the fourth embodiment, R¹³ is selected from the group consisting of methyl, ethyl, methylphenyl, ethoxyethyl, methoxyethyl, methoxycarbonylcyclopropyl, 1-carboxyl 2-methoxypropyl and methoxyacetyl radicals, which radicals may be substituted with methyl, and hydroxy radicals, and NR¹⁵ is selected from the group consisting of N-piperidyl, N-morpholinyl, N-piperazinyl, N-pyrollidinyl and 1,4 sulfone hexazane radicals wherein such radicals may be substituted with methyl, hydroxy, carboxylmethyl, sulfonic acid and ethyloxyethyl radicals.

Such modifications are intended to fall within the scope of the appended claims.

All references cited herein are hereby incorporated by reference in their entirety.

The foregoing description details specific methods and compositions that can be employed to practice the present invention, and represents the best mode contemplated. However, it is apparent for one of ordinary skill in the art that further compounds with the desired pharmacological properties can be prepared in an analogous manner, and that the disclosed compounds can also be obtained from different starting compounds via different chemical reactions. Similarly, different pharmaceutical compositions may be prepared and used with substantially the same result. Thus, however detailed the foregoing may appear in text, it should not be construed as limiting the overall scope hereof; rather, the ambit of the present invention is to be governed only by the lawful construction of the appended claims.

What is claimed:

1. A compound represented by the general formula II 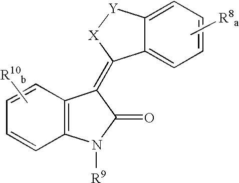
wherein X is O; Y is [C(R⁹)₂]_{c}; R¹⁰ is selected from the group consisting of halogen, nitro, hydroxy, hydrocarbyl, substituted hydrocarbyl, amide, thioamide, amine, thioether and sulfonyl; R⁸ is selected from the group consisting of halogen, nitro, hydroxy, hydrocarbyl, substituted hydrocarbyl, amide, thioamide, amine, thioether and sulfonyl and phosphonic acid; R⁹ is selected from the group consisting of hydrogen, hydrocarbyl and substituted hydrocarbyl; c is an integer of from 1 to 2; b is 0 or an integer from 1 to 3; a is 0 or an integer of from 1 to 3 and pharmaceutically acceptable salts thereof

2. The compound of claim 1 wherein a is 0 and Y is HCCOOH.

3. The compound of claim 1 wherein c is 1.

4. The compound of claim 3 wherein b is 1 and R¹⁰ is R¹⁴ and is selected from the group consisting of hydrogen, fluoro and chloro.

5. The compound of claim 4 wherein a is 1.

6. The compound of claim 5 wherein R⁹ is R¹⁶ and is independently selected from the group consisting of hydrogen, lower alkyl, carboxylic, phenyl, thienyl, tetrazolyl and morpholinyl-N-methyl radicals.

7. The compound of claim 6 wherein R⁸ is R¹² and R¹² is is selected from the group consisting of R¹³, OR¹³, 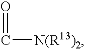
(CH₂)ₐN(R¹³)₂ and C≡C—CH₂—N(R¹³)₂ wherein R¹³ is selected from the group consisting of alkyl and phenyl radicals which may be substituted with heteroatoms provided by halogen or nitrogen, oxygen or sulfur-containing radicals or said phenyl radicals may be substituted with alkyl radicals.

8. The compound of claim 7 wherein R¹² is R¹³.

9. The compound of claim 8 wherein R¹³ is selected from the group consisting alkyl radicals which are substituted with heteroatoms selected from the group consisting of nitrogen, oxygen and sulfur.

10. The compound of claim 9 wherein R¹³ may include 1, 2, 3, 4 or 5 oxygen atoms and/or 1 nitrogen atom and/or 1 sulfur atom.

11. The compound of claim 10 wherein R¹³ is selected from the group consisting of hydroxypropyl, methylsulfoxypropyl, aminopropyl, acetyl, 2,2-dimethoxyethyl, hydroxyethenyl, 2-(1,2 dioxyethylenyl)ethyl, carboxy ethyl and N-ethylene oxide adducts of ethylcarbamic acid.

12. The compound of claim 7 wherein R¹² is OR¹³ wherein R¹³ is selected from the group consisting of alkyl radicals, which are substituted with heteroatoms selected from the group consisting of nitrogen and oxygen.

13. The compound of claim 12 wherein OR¹³ is N-ethylmorpholinyl.

14. The compound of claim 7 wherein R¹² is 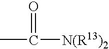
wherein R¹³ is selected from the group consisting of alkyl radicals which are substituted with heteroatoms selected from the group consisting of nitrogen and oxygen.

15. The compound of claim 14 wherein R¹³ is selected from the group consisting of 2-(diethylamino)ethyl, 2-(dimethylamino)ethyl, 2-(N-morpholinyl)ethyl, 2-(N-piperidinyl)ethyl and N-ethylene oxide adducts.

16. The compound of claim 7 wherein R¹² is (CH₂)ₐN(R¹³)₂.

17. The compound of claim 16 wherein a is 0 and R¹² is N(R¹³)₂ wherein R¹³ is selected from the group consisting of alkyl radicals which are substituted with heteroatoms selected from the group consisting of nitrogen, oxygen and halogen radicals.

18. The compound of claim 17 wherein N(R¹³)₂ is N(Et)₂ wherein Et is ethyl and may be substituted with one chloro or hydroxyl radical or N(R¹³)₂ is 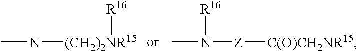
wherein Z is CH₂—CH₂—O or is absent, R¹⁶ is H or methyl and R¹⁵ together with N forms a N-cycloalkyl radical, which cycloalkyl radical may be substituted with one or more lower alkyl radicals, or halogen radicals.

19. The compound of claim 18 wherein NR¹⁵ is piperidine.

20. The compound of claim 18 wherein NR¹⁵ is morpholine.

21. The compound of claim 18 wherein NR¹⁵ is piperazine.

22. The compound of claim 17 wherein one of R¹³ is selected from the group consisting of ethyl, 2-hydroxyethyl, 2-chloroethyl, 2-N-piperidylethyl, 2-N(4-fluoropiperidlyl)ethyl, 2-N-morpholinylethyl, 2-N-(3,5-dimethyl morpholinyl)ethyl, 2-N(4-methyl piperazinyl)ethyl, 2-N pyrollidinylethyl, 2-diethylaminoethyl, piperidylmethylcarbonyl, N 4-methylpiperazinylmethylcarbonyl, acetoxyethyl, bromoacetoxyethyl, N-morpholinyl acetoxyethyl, diethylamino-acetoxyethyl, [N-methyl(N-piperazyl)]acetoxyethyl and N-piperidyl acetoxyethyl, and N-morpholinylmethylcarbonyl.

23. The compound of claim 7 wherein a is 1, 2 or 3 and R¹² is (CH₂)ₐN(R¹³)₂ or (CH₂)ₐNR¹⁵ wherein R¹³ is selected from the group consisting of phenyl radicals, alkyl-substituted phenyl radicals, alkyl radicals, which alkyl radicals may be substituted with hetero atoms selected from the group consisting of nitrogen, oxygen or sulfur radicals and R¹⁵, together with N forms a N-cycloalkyl radical, which may be substituted with carboxylalkyl, sulfonic acid, hydroxy, or carboxylic radicals and/or said cycloalkyl radical may include an additional enchained nitrogen atom or an oxygen or a sulfur atom.

24. The compound of claim 23 wherein, R¹³ is selected from the group consisting of methyl, ethyl, methylphenyl, ethoxyethyl, methoxyethyl, methoxycarbonylcyclopropyl, 1-carboxyl 2-methoxypropyl and methoxyacetyl radicals, which radicals may be substituted with methyl and hydroxyl radicals, and NR¹⁵ is selected from the group consisting of N-piperidyl, N-morpholinyl, N-piperazinyl and N-pyrollidinyl radicals wherein such radicals may be substituted with methyl, hydroxy, carboxylmethyl, sulfonic acid and ethyloxyethyl radicals.

25. A method for treating diseases related to unregulated tyrosine kinase signal transduction, the method comprising the step of administering to a subject in need thereof a therapeutically effective amount of a compound according to claim 1.

26. The method of claim 25 wherein said disease is selected from the group consisting of cancer, blood vessel proliferative disorders, fibrotic disorders, mesangial cell proliferative disorders and metabolic diseases.

27. The method of claim 26 wherein the blood vessel proliferative disorder is selected from the group consisting of diabetic retinopathy, age-related macular degeneration, retinopathy of prematurity, arthritis and restenosis.

28. The method of claim 26 wherein the fibrotic disorder is selected from the group consisting of hepatic cirrhosis, atherosclerosis and surgical adhesions.

29. The method of claim 26 wherein the mesangial cell proliferative disorder is selected from the group consisting of glomerulonephritis, diabetic nephropathy, malignant nephrosclerosis, thrombotic microangiopathy syndromes, transplant rejection and glomerulopathies.

30. The method of claim 11 wherein the metabolic disorder is selected from the group consisting of psoriasis, diabetes mellitus, wound healing, inflammation and neurodegenerative diseases.
